SEQUENCE LISTING (57 sequences)

SEQ ID NO: 1 - protein, 5 aa, synthetic construct
YSWIN

SEQ ID NO: 2 - protein, 17 aa, synthetic construct
RIFPGDGDTDYNGKFKG

SEQ ID NO: 3 - protein, 10 aa, synthetic construct
NVFDGYWLVY

SEQ ID NO: 4 - protein, 16 aa, synthetic construct
RSSKSLLHSNGITYLY

SEQ ID NO: 5 - protein, 7 aa, synthetic construct
QMSNLVS

SEQ ID NO: 6 - protein, 9 aa, synthetic construct
AQNLELPYT

SEQ ID NO: 7 - protein, 119 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 8 - protein, 112 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIK

SEQ ID NO: 9 - protein, 5 aa, synthetic construct
TYAMN

SEQ ID NO: 10 - protein, 19 aa, synthetic construct
RIRSKYNNYATYYADSVKG

SEQ ID NO: 11 - protein, 14 aa, synthetic construct
HGNFGNSYVSWFAY

SEQ ID NO: 12 - protein, 14 aa, synthetic construct
GSSTGAVTTSNYAN

SEQ ID NO: 13 - protein, 7 aa, synthetic construct
GTNKRAP

SEQ ID NO: 14 - protein, 9 aa, synthetic construct
ALWYSNLWV

SEQ ID NO: 15 - protein, 125 aa, synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 16 - protein, 109 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 17 - protein, 672 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 18 - protein, 447 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 19 - protein, 232 aa, synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 219 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 5 aa, synthetic construct
GGGGS

SEQ ID NO: 22 - protein, 5 aa, synthetic construct
SGGGG

SEQ ID NO: 23 - protein, 9 aa, synthetic construct
GGGGSGGGG

SEQ ID NO: 24 - protein, 20 aa, synthetic construct
GGGGSGGGGSGGGGSGGGGS

SEQ ID NO: 25 - protein, 12 aa, synthetic construct
GGGSGGGSGGGS

SEQ ID NO: 26 - protein, 16 aa, synthetic construct
GGGSGGGSGGGSGGGS

SEQ ID NO: 27 - protein, 20 aa, synthetic construct
GGGSGGGSGGGSGGGSGGGS

SEQ ID NO: 28 - protein, 24 aa, synthetic construct
GGGSGGGSGGGSGGGSGGGSGGGS

SEQ ID NO: 29 - protein, 10 aa, synthetic construct
GGGGSGGGGS

SEQ ID NO: 30 - protein, 15 aa, synthetic construct
GGGGSGGGGSGGGGS

SEQ ID NO: 31 - protein, 20 aa, synthetic construct
GGGGSGGGGSGGGGSGGGGS

SEQ ID NO: 32 - protein, 25 aa, synthetic construct
GGGGSGGGGSGGGGSGGGGSGGGGS

SEQ ID NO: 33 - protein, 13 aa, synthetic construct
GGGSGGGSGGGSG

SEQ ID NO: 34 - protein, 17 aa, synthetic construct
GGGSGGGSGGGSGGGSG

SEQ ID NO: 35 - protein, 21 aa, synthetic construct
GGGSGGGSGGGSGGGSGGGSG

SEQ ID NO: 36 - protein, 25 aa, synthetic construct
GGGSGGGSGGGSGGGSGGGSGGGSG

SEQ ID NO: 37 - protein, 11 aa, synthetic construct
GGGGSGGGGSG

SEQ ID NO: 38 - protein, 16 aa, synthetic construct
GGGGSGGGGSGGGGSG

SEQ ID NO: 39 - protein, 21 aa, synthetic construct
GGGGSGGGGSGGGGSGGGGSG

SEQ ID NO: 40 - protein, 26 aa, synthetic construct
GGGGSGGGGSGGGGSGGGGSGGGGSG

SEQ ID NO: 41 - protein, 14 aa, synthetic construct
GGGSGGGSGGGSGG

SEQ ID NO: 42 - protein, 18 aa, synthetic construct
GGGSGGGSGGGSGGGSGG

SEQ ID NO: 43 - protein, 22 aa, synthetic construct
GGGSGGGSGGGSGGGSGGGSGG

SEQ ID NO: 44 - protein, 26 aa, synthetic construct
GGGSGGGSGGGSGGGSGGGSGGGSGG

SEQ ID NO: 45 - protein, 12 aa, synthetic construct
GGGGSGGGGSGG

SEQ ID NO: 46 - protein, 17 aa, synthetic construct
GGGGSGGGGSGGGGSGG

SEQ ID NO: 47 - protein, 22 aa, synthetic construct
GGGGSGGGGSGGGGSGGGGSGG

SEQ ID NO: 48 - protein, 27 aa, synthetic construct
GGGGSGGGGSGGGGSGGGGSGGGGSGG

SEQ ID NO: 49 - protein, 15 aa, synthetic construct
GGGSGGGSGGGSGGG

SEQ ID NO: 50 - protein, 19 aa, synthetic construct
GGGSGGGSGGGSGGGSGGG

SEQ ID NO: 51 - protein, 23 aa, synthetic construct
GGGSGGGSGGGSGGGSGGGSGGG

SEQ ID NO: 52 - protein, 27 aa, synthetic construct
GGGSGGGSGGGSGGGSGGGSGGGSGGG

SEQ ID NO: 53 - protein, 13 aa, synthetic construct
GGGGSGGGGSGGG

SEQ ID NO: 54 - protein, 18 aa, synthetic construct
GGGGSGGGGSGGGGSGGG

SEQ ID NO: 55 - protein, 23 aa, synthetic construct
GGGGSGGGGSGGGGSGGGGSGGG

SEQ ID NO: 56 - protein, 28 aa, synthetic construct
GGGGSGGGGSGGGGSGGGGSGGGGSGGG

SEQ ID NO: 57 - protein, 11 aa, synthetic construct
DGGGGSGGGGS

(19) United States
(12) Patent Application Publication
(10) Pub. No.: US 2023/0414750 A1
(43) Pub. Date: Dec. 28, 2023

(54) COMBINATION TREATMENT OF AN ANTI-CD20/ANTI-CD3 BISPECIFIC ANTIBODY AND CHEMOTHERAPY

(71) Applicants: Hoffmann-La Roche Inc., Little Falls, NJ (US); Genentech, Inc., South San Francisco, CA (US)

(72) Inventors: Maria FILIPPOU-FRYE, Brisbane, CA (US); Joseph Nathaniel PAULSON, San Francisco, CA (US); Leonardo ROQUE PEREIRA, Bishop's Stortford (GB); Emily PICCIONE GRIFFIN, Belmont, CA (US); Stephen James SIMKO, III, San Francisco, CA (US); Johanna SY, Pacifica, CA (US); Akiko TAGAWA, Greifensee (CH); Beate WULFF, Hamburg (DE); Betsy Lane ALTHAUS, San Carlos, CA (US); David CARLILE, Princes Risborough (GB); Nassim DJEBLI, Rixheim (FR)

(21) Appl. No.: 18/188,168

(22) Filed: Mar. 22, 2023

Related U.S. Application Data
(60) Provisional application No. 63/402,453, filed on Aug. 30, 2022.
(60) Provisional application No. 63/322,780, filed on Mar. 23, 2022.

(51) Int. Cl. A61K 39/395; A61P 35/00; A61K 31/664; A61K 31/282; A61K 31/7048; A61K 38/19; A61K 31/185; A61K 31/573; A61K 31/138; A61K 31/167; C07K 16/28

(52) U.S. Cl. CPC A61K 39/3955; A61P 35/00; A61K 31/664; A61K 31/282; A61K 31/7048; A61K 38/193; A61K 2039/545; A61K 31/573; A61K 31/138; A61K 31/167; C07K 16/2887; C07K 16/2809; A61K 31/185

(57) ABSTRACT

The present invention relates to methods of treating B-cell proliferative disorders, e.g., primary refractory or relapsed diffuse large B-cell lymphoma (DLBCL), by administering an anti-CD20/anti-CD3 bispecific antibody and in combination with an anti-CD20 antibody (e.g., obinutuzumab or rituximab) and one or more chemotherapeutic agents selected from ifosfamide, carboplatin, and/or etoposide.

### SEQUENCE LISTING

The instant application contains a Sequence Listing which has been submitted electronically in XML format and is hereby incorporated by reference in its entirety. Said XML copy, created on Feb. 16, 2023, is named “P37449-sequence listing-20230216.xml” and is 52,509 bytes in size.

### FIELD OF THE INVENTION

The present invention relates to methods of treating B-cell proliferative disorders, e.g., primary refractory or relapsed diffuse large B-cell lymphoma (DLBCL), by administering an anti-CD20/anti-CD3 bispecific antibody and in combination with an anti-CD20 antibody (e.g., obinutuzumab or rituximab) and one or more chemotherapeutic agents selected from ifosfamide, carboplatin and/or etoposide.

### BACKGROUND OF THE INVENTION

Non-Hodgkin lymphoma (NHL) is the most common hematologic malignancy in the world (Bray et al. 2018). The most common subtype of NHL of B-cell origin (Sun et al. Am. J. Clin. Pathol. 138:429-434, 2012; Al-Hamadani et al. Am. J. Hematol. 24:4785-4797, 2015), DLBCL is an aggressive NHL with a median survival of <1 year in untreated patients (Rovira et al. Ann. Hematol. 94:803-812, 2015). Despite its aggressive disease course, approximately 50%-70% of patients may be cured with the current standard-of-care treatment that consists of rituximab, a monoclonal antibody targeting CD20, in combination with cyclophosphamide, doxorubicin, vincristine, and prednisone (R-CHOP) chemotherapy (Flowers et al. CA Cancer J. Clin. 60:393-408 2010; Tilly et al. Ann. Oncol. 26(Suppl 5):v116-125 2015; NCCN Clinical Practice Guidelines in Oncology, 2020).

Nevertheless, R-CHOP is found to be inadequate in 30%-50% of patients because of either primary refractoriness, defined as failure to achieve a complete response (CR) after first-line therapy with rituximab plus an anthracycline (Vardhana et al. Br. J. Haematol. 176:591-599, 2017), or relapse after achieving a CR. For patients who are not cured by first-line therapy and who are medically able to tolerate intensive therapy, high-dose salvage chemoimmunotherapy followed by ASCT offers a second chance for long-term remission. Approximately half of patients with relapsed DLBCL are refractory to salvage chemoimmunotherapy (Gisselbrecht et al. J. Clin. Oncol. 28:4184-4190, 2010) and are thus unable to proceed to ASCT. In addition, for a subgroup of patients with DLBCL, CAR-T therapy is an available treatment option, particularly for patients that have primary refractory disease, or have relapsed within 12 months of initial chemoimmunotherapy (Kamdar et al. 2022, Lancet 399, 2294-2308; Locke et al. 2022, N Engl J Med 386, 640-54). Given that the median EFS observed in both studies was less than 1 year, continued opportunity for optimization of therapeutic regimens involving CAR-T therapy remains. Therefore, a significant clinical need exists for improved salvage immunochemotherapy regimens for patients with relapsed or refractory (R/R) DLBCL.

Combining an anti-CD20/anti-CD3 bispecific antibody with an anti-CD20 antibody (e.g., obinutuzumab or rituximab) and one or more chemotherapeutic agents selected from ifosfamide, carboplatin and/or etoposide may provide improved response rates to salvage chemoimmunotherapy which may eventually translate into a higher percentage of patients receiving definitive therapy with ASCT or CAR-T therapy and improved survival in this treatment setting.

Aggressive mature B-NHL accounts for approximately 60% of all cases of childhood Non-Hodgkin lymphoma (NHL). Main histological subtypes are Burkitt lymphoma (BL), Burkitt leukemia (BAL; analogous to mature B-cell leukemia FAB L3), diffuse large B-cell lymphoma (DLBCL), primary mediastinal large B-cell lymphoma (PMBCL), and aggressive mature B-NHL not further classifiable. While frontline therapy with intensive chemotherapy plus rituximab is highly effective in children, with 3-year event-free survival of 94% (Minard-Colin et al. N. Eng. J. Med. 382:2207-2219, 2020), there is still an urgent and high unmet need for the development of an effective salvage regimen for patients with first R/R disease (Pearson et al. Eur. J. Cancer. 110:74-85, 2019). The probability of survival for R/R B-NHL in children, adolescents, and young adults is very poor with 1-year overall survival (OS) rates below 30% (Cairo et al. Br. J. Haematol. 182:859-869, 2018; Woessmann et al. Blood. 135:1124-1132, 2020; Burkhardt et al. Cancers. 13:2075, 2021; Crombie and LaCasce Blood. 137:743-750, 2021).

### SUMMARY OF THE INVENTION

In one aspect, the invention features a method of treating a subject having a CD20-positive cell proliferative disorder comprising administering to the subject an effective amount of
(a) a bispecific antibody that binds to CD20 and CD3;
(b) an anti-CD20 antibody; and
(c) one or more chemotherapeutic agents selected from ifosfamide, carboplatin and/or etoposide in a dosing regimen comprising at least a first dosing cycle and a second dosing cycle.

In one embodiment, the subject is aged 18 years or older (e.g., 20, 25, 30, 35, 40, 45, 50, 55, 60, 65, 70, 75, 80, 85, 90, 95, 100, 105, or 110 years or older). In one embodiment, the subject is aged 31 years or older.

In one embodiment, the first dosing cycle comprises a first dose (C1D1) of the bispecific antibody that binds to CD20 and CD3 and a second dose (C1D2) of the bispecific antibody, wherein the C1D1 of the bispecific antibody is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of the bispecific antibody is about 10 mg (e.g., 10 mg±0.05 mg, ±0.1 mg, ±0.2 mg, ±0.3 mg, ±0.5 mg, ±0.75 mg, or ±1 mg); and the second dosing cycle comprises a single dose (C2D1) of the bispecific antibody, wherein the C2D1 of the bispecific antibody is about 10 mg (e.g., 10 mg±0.05 mg, ±0.1 mg, ±0.2 mg, ±0.3 mg, ±0.5 mg, ±0.75 mg, or ±1 mg), about 16 mg (e.g., 16 mg±0.05 mg, ±0.1 mg, ±0.2 mg, ±0.3 mg, ±0.5 mg, ±0.75 mg, ±1 mg, ±1.5 mg, or ±1.6 mg), or about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg). In one embodiment, the C2D1 of the bispecific antibody is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one embodiment, the first dose (C1D1) of the bispecific antibody that binds to CD20 and CD3 and the second dose (C1D2) of the bispecific antibody that binds to CD20 and CD3 are administered to the subject on Days 8 and 15, respectively, of the first dosing cycle. In one embodiment, the C2D1 of the bispecific antibody that binds to CD20 and CD3 is administered to the subject on Day 8 of the second dosing cycle.

In one embodiment, the anti-CD20 antibody is obinutuzumab and/or rituximab. In one embodiment, the first dosing cycle comprises a single dose (C1D1) of obinutuzumab; and the second dosing cycle comprises a single dose (C2D1) of rituximab. In one embodiment, the single dose C1D1 of the obinutuzumab is about 1000 mg (e.g., 1000 mg±5 mg, ±10 mg, ±20 mg, ±30 mg, ±50 mg, ±75 mg, or ±100 mg) and the single dose of rituximab is about 375 mg/m² (e.g., 375 mg/m²±5 mg/m², ±10 mg/m², ±25 mg/m², or ±37.5 mg/m²). In one embodiment, the anti-CD20 antibody is administered in a dosing regimen comprising at least a first dosing cycle and a second dosing cycle, wherein the first dosing cycle comprises a single dose (C1D1) of obinutuzumab on Day 1; and the second dosing cycle comprises a single dose (C2D1) of rituximab on Day 1.

In one embodiment, step c) comprises all three chemotherapeutic agents. In one embodiment, the first dosing cycle comprises a single dose (C1D1) of ifosfamide, a single dose (C1D1) of carboplatin and a first (C1D1), second (C1D2) and third (C1D3) dose of etoposide; and the second cycle each comprises a single dose (C2D1) of ifosfamide, a single dose (C2D1) of carboplatin and a first (C2D1), second (C2D2) and third (C2D3) dose of etoposide. In one embodiment, ifosfamide is administered at a dose of about 5000 mg/m² (e.g., 5000 mg/m²±50 mg/m², ±100 mg/m², ±200 mg/m², ±300 mg/m², ±400 mg/m², or ±500 mg/m²), about 4000 mg/m² (e.g., 4000 mg/m²±40 mg/m², ±50 mg/m², ±100 mg/m², ±200 mg/m², ±300 mg/m², or ±400 mg/m²), or about 1666 mg/m² (e.g., 1666 mg/m²±25 mg/m², ±50 mg/m², ±100 mg/m², or ±166.6 mg/m²), carboplatin is administered at a dose in mg to target area under the curve (AUC) of about 5 mg/mL/min (e.g., 5 mg/mL/min±0.05 mg/mL/min, ±0.1 mg/mL/min, ±0.25 mg/mL/min, or ±0.5 mg/mL/min) with maximum dose of about 750 mg (e.g., 750 mg±10 mg, ±25 mg, ±50 mg, or ±75 mg) and etoposide is administered at a dose of about 100 mg/m² (e.g., 100 mg/m²±1 mg/m², ±2.5 mg/m², ±5 mg/m², or ±10 mg/m²) or 75 mg/m² (e.g., 0.5 mg/m²±1 mg/m², ±2.5 mg/m², ±5 mg/m², or ±7.5 mg/m²). In one embodiment, ifosfamide is administered at a dose of 5000 mg/m², 4000 mg/m² or 1666 mg/m², carboplatin is administered at a dose in mg to target area under the curve (AUC) of 5 mg/mL/min with maximum dose of 750 mg and etoposide is administered at a dose of 100 mg/m² or 75 mg/m². In one embodiment, ifosfamide is administered at a dose of 5000 mg/m², carboplatin is administered at a dose in mg to target area under the curve (AUC) of 5 mg/mL/min with maximum dose of 750 mg and etoposide is administered at a dose of 100 mg/m². In one embodiment, ifosfamide and carboplatin are administered on Day 2 of the first and second dosing cycle and etoposide is administered on each of Days 1, 2 and 3 of the first and second dosing cycle.

In one embodiment, the first and second dosing cycles are 21-day dosing cycles.

In one embodiment, the dosing regimen comprises one or more additional dosing cycles. In one embodiment, the additional dosing cycles are 21-day dosing cycles. In one embodiment, the dosing regimen comprises three dosing cycles in total.

In one embodiment, the one or more additional dosing cycles comprise
(a) an additional single dose of the bispecific antibody that binds to CD20 and CD3,
(b) an additional single dose of the anti-CD20 antibody, and
(c) an additional single dose of ifosfamide, an additional single dose of carboplatin and an additional first, second and third dose of etoposide. In one embodiment, the additional single dose of the bispecific antibody is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one embodiment, the anti-CD20 antibody of the one or more additional dosing cycles is rituximab. In one embodiment, the additional single dose of rituximab is about 375 mg/m² (e.g., 375 mg/m²±5 mg/m², ±10 mg/m², ±25 mg/m², or ±37.5 mg/m²). In one embodiment, the additional single dose of rituximab is administered on Day 1 of the additional dosing cycle.

In one embodiment, the additional single dose of ifosfamide is about 5000 mg/m² (e.g., 5000 mg/m²±50 mg/m², ±100 mg/m², ±200 mg/m², ±300 mg/m², ±400 mg/m², or ±500 mg/m²), about 4000 mg/m² (e.g., 4000 mg/m²±40 mg/m², ±50 mg/m², ±100 mg/m², ±200 mg/m², ±300 mg/m², or ±400 mg/m²), or about 1666 mg/m² (e.g., 1666 mg/m²±25 mg/m², ±50 mg/m², ±100 mg/m², or ±166.6 mg/m²), the additional single dose of carboplatin is in mg to target area under the curve (AUC) of about 5 mg/mL/min (e.g., 5 mg/mL/min±0.05 mg/mL/min, ±0.1 mg/mL/min, ±0.25 mg/mL/min, or ±0.5 mg/mL/min) with maximum dose of about 750 mg (e.g., 750 mg±10 mg, ±25 mg, ±50 mg, or ±75 mg) and the additional first, second and third dose of etoposide is 100 mg/m² (e.g., 100 mg/m²±1 mg/m², ±2.5 mg/m², ±5 mg/m², or ±10 mg/m²) or 75 mg/m² (e.g., 0.5 mg/m²±1 mg/m², ±2.5 mg/m², ±5 mg/m², or ±7.5 mg/m²). In one embodiment, the additional single dose of ifosfamide is 5000 mg/m², 4000 mg/m² or 1666 mg/m², the additional single dose of carboplatin is in mg to target area under the curve (AUC) of 5 mg/mL/min with maximum dose of 750 mg and the additional first, second and third dose of etoposide is 100 mg/m² or 75 mg/m². In one embodiment, ifosfamide is administered at a dose of 5000 mg/m², carboplatin is administered at a dose in mg to target area under the curve (AUC) of 5 mg/mL/min with maximum dose of 750 mg and etoposide is administered at a dose of 100 mg/m².

In one embodiment, ifosfamide and carboplatin are administered on Day 2 of the additional dosing cycle and etoposide is administered on each of Days 1, 2 and 3 of the additional dosing cycle.

In one embodiment, the method further comprises administering to the subject one or more additional therapeutic agents. In one embodiment, the one or more additional therapeutic agent is tocilizumab. In one embodiment, the weight of the subject is greater than or equal to about 30 kg, and tocilizumab is administered at a dose of about 8 mg/kg (e.g., 8 mg/kg±0.05 mg/kg, ±0.1 mg/kg, ±0.25 mg/kg, ±0.5 mg/kg, or ±0.8 mg/kg). In one embodiment, the weight of the subject is less than 30 kg, and tocilizumab is administered at a dose of about 12 mg/kg (e.g., 12 mg/kg±0.05 mg/kg, ±0.1 mg/kg, ±0.25 mg/kg, ±0.5 mg/kg, ±0.75 mg/kg, ±1 mg/kg, or ±1.2 mg/kg). In some embodiments, the maximum dose of tocilizumab is about 800 mg (e.g., 800 mg±10 mg, ±25 mg, ±50 mg, or ±80 mg).

In one embodiment, the one or more additional therapeutic agents is a corticosteroid. In one embodiment, the corticosteroid comprises prednisone, prednisolone, methylprednisolone, or dexamethasone. In one embodiment, dexamethasone is administered intravenously at a dose of about 20 mg (e.g., 20 mg±0.1 mg, ±0.25 mg, ±0.5 mg, ±1 mg, ±1.5 mg, or ±2 mg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the bispecific antibody. In one embodiment, dexamethasone is administered intravenously at a dose of about 20 mg (e.g., 20 mg±0.1 mg, ±0.25 mg, ±0.5 mg, ±1 mg, ±1.5 mg, or ±2 mg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of obinutuzumab. In one embodiment, wherein methylprednisolone is administered intravenously at a dose of about 80 mg (e.g., 80 mg±0.5 mg, ±1 mg, ±1.5 mg, ±2 mg, ±4 mg, ±6 mg, or ±8 mg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the bispecific antibody. In one embodiment, methylprednisolone is administered intravenously at a dose of about 80 mg (e.g., 80 mg±0.5 mg, ±1 mg, ±1.5 mg, ±2 mg, ±4 mg, ±6 mg, or ±8 mg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of obinutuzumab. In one embodiment, prednisone is administered orally at a dose of about 100 mg (e.g., 100 mg±0.5 mg, ±1 mg, ±1.5 mg, ±2 mg, ±4 mg, ±6 mg, ±8 mg, or ±10 mg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the bispecific antibody. In one embodiment, prednisolone is administered intravenously at a dose of about 100 mg (e.g., 100 mg±0.5 mg, ±1 mg, ±1.5 mg, ±2 mg, ±4 mg, ±6 mg, ±8 mg, or ±10 mg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the bispecific antibody.

In one embodiment, the one or more additional therapeutic agents is an antihistamine. In one embodiment, the antihistamine is diphenhydramine. In one embodiment, diphenhydramine is administered orally or intravenously at a dose of about 50 mg (e.g., 50 mg±0.5 mg, ±1 mg, ±1.5 mg, ±2 mg, ±3 mg, ±4 mg, or ±5 mg) at least about 30 minutes (i.e., at least 30 minutes±3 minutes; e.g., at least about 1, 2, 3, 4, 5, 6, 7, 8, 9, 10, 11, 12, 18, 24, 36, 48 hours, or more) prior to the administration of any dose of the bispecific antibody.

In one embodiment, the one or more additional therapeutic agents comprises granulocyte-colony stimulating factor (G-CSF). In one embodiment, G-CSF is administered between about one day and about two days (e.g., 24, 26, 28, 30, 32, 36, 38, 40, 42, 44, 46, or 48 hours) after administration of any dose of rituximab, ifosfamide, carboplatin, and/or etoposide.

In one embodiment, the one or more additional therapeutic agents is an antipyretic. In one embodiment, the antipyretic is acetaminophen or paracetamol. In one embodiment, acetaminophen or paracetamol is administered orally at a dose of between about 500 mg to about 1000 mg (e.g., 1000 mg±5 mg, ±10 mg, ±20 mg, ±30 mg, ±50 mg, ±75 mg, or ±100 mg) at least about 30 minutes (i.e., at least 30 minutes±3 minutes; e.g., at least about 1, 2, 3, 4, 5, 6, 7, 8, 9, 10, 11, 12, 18, 24, 36, 48 hours, or more) prior to the administration of any dose of the bispecific antibody. In one embodiment, acetaminophen or paracetamol is administered orally at a dose of between about 500 mg to about 1000 mg (e.g., 1000 mg±5 mg, ±10 mg, ±20 mg, ±30 mg, ±50 mg, ±75 mg, or ±100 mg) at least about 30 minutes (i.e., at least 30 minutes±3 minutes; e.g., at least about 1, 2, 3, 4, 5, 6, 7, 8, 9, 10, 11, 12, 18, 24, 36, 48 hours, or more) prior to the administration of any dose of obinutuzumab.

In one embodiment, the one or more additional therapeutic agents is mesna. In one embodiment, mesna is administered at a dose of about 5000 mg/m² (e.g., 5000 mg/m²±50 mg/m², ±100 mg/m², ±200 mg/m², ±300 mg/m², ±400 mg/m², or ±500 mg/m²), about 4000 mg/m² (e.g., 4000 mg/m²±40 mg/m², ±50 mg/m², ±100 mg/m², ±200 mg/m², ±300 mg/m², or ±400 mg/m²), or about 1666 mg/m² (e.g., 1666 mg/m²±25 mg/m², ±50 mg/m², ±100 mg/m², or ±166.6 mg/m²) intravenously.

In one aspect, the invention features a method of treating a subject aged between 6 months and 17 years having a CD20-positive cell proliferative disorder comprising administering to the subject an effective amount of
(a) a bispecific antibody that binds to CD20 and CD3;
(b) an anti-CD20 antibody; and
(c) one or more chemotherapeutic agents selected from ifosfamide, carboplatin, and/or etoposide in a dosing regimen comprising at least a first dosing cycle and a second dosing cycle.

In one embodiment, the first dosing cycle comprises a first dose (C1D1) of the bispecific antibody and a second dose (C1D2) of the bispecific antibody, wherein the C1D1 of the bispecific antibody is about 0.03 mg/kg (e.g., 0.03 mg/kg±0.0005 mg/kg, ±0.001 mg/kg, ±0.002 mg/kg, or ±0.003 mg/kg), about 0.03 mg/kg (e.g., 0.04 mg/kg±0.0005 mg/kg, ±0.001 mg/kg, ±0.002 mg/kg, ±0.003 mg/kg, or ±0.004 0.04 mg/kg), or about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of the bispecific antibody is about 0.15 mg/kg (e.g., 0.15 mg/kg±0.001 mg/kg, ±0.0025 mg/kg, ±0.005 mg/kg, ±0.01 mg/kg, or ±0.015 mg/kg) or about 10 mg (e.g., 10 mg±0.05 mg, ±0.1 mg, ±0.2 mg, ±0.3 mg, ±0.5 mg, ±0.75 mg, or ±1 mg); and
the second dosing cycle comprises a single dose (C2D1) of the bispecific antibody, wherein the C2D1 of the bispecific antibody is about 0.4 mg/kg (e.g., 0.4 mg/kg±0.005 mg/kg, ±0.01 mg/kg, ±0.02 mg/kg, ±0.03 mg/kg, or ±0.04 mg/kg), about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg), or about 30 mg (e.g., 30 mg±0.1 mg, ±0.2 mg, ±0.3 mg, ±0.5 mg, ±0.75 mg, ±1 mg, ±1.5 mg, ±2 mg, or 3 mg).

In one embodiment, (a) the subject's body weight is greater than or equal to about 7.5 kg and less than about 13 kg, and wherein the C1D1 of the bispecific antibody is about 0.04 mg/kg (e.g., 0.04 mg/kg±0.0005 mg/kg, ±0.001 mg/kg, ±0.002 mg/kg, ±0.003 mg/kg, or ±0.004 mg/kg), the C1D2 of the bispecific antibody is about 0.15 mg/kg (e.g., 0.15 mg/kg±0.001 mg/kg, ±0.0025 mg/kg, ±0.005 mg/kg, ±0.01 mg/kg, or ±0.015 mg/kg), and the C2D1 of the bispecific antibody is about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg);
(b) the subject's body weight is greater than or equal to about 13 kg and less than about 45 kg, and wherein the C1D1 of the bispecific antibody is about 0.03 mg/kg (e.g., 0.03 mg/kg±0.0005 mg/kg, ±0.001 mg/kg, ±0.002 mg/kg, or ±0.003 mg/kg), the C1D2 of the bispecific antibody is about 0.15 mg/kg (e.g., 0.15 mg/kg±0.001 mg/kg, ±0.0025 mg/kg, ±0.005 mg/kg, ±0.01 mg/kg, or ±0.015 mg/kg), and the C2D1 of the bispecific antibody is about 0.4 mg/kg (e.g., 0.4 mg/kg±0.005 mg/kg, ±0.01 mg/kg, ±0.02 mg/kg, ±0.03 mg/kg, or ±0.04 mg/kg); or
(c) the subject's body weight is greater than or equal to about 45 kg, and wherein the C1D1 of the bispecific antibody is about 2.5 mg (e.g., 2.5 mg±0.01 mg, ±0.02 mg, ±0.03 mg, ±0.05 mg, ±0.1 mg, ±0.2 mg, or ±0.25 mg), the C1D2 of the bispecific antibody is about 10 mg (e.g., 10 mg±0.05 mg, ±0.1 mg, ±0.2 mg, ±0.3 mg, ±0.5 mg, ±0.75 mg, or ±1 mg), and the C2D1 of the bispecific antibody is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one embodiment, the C1D1 of the bispecific antibody and the C1D2 of the bispecific antibody are administered to the subject on Days 8 and 15, respectively, of the first dosing cycle. In one embodiment, the C2D1 of the bispecific antibody is administered to the subject on Day 1 of the second dosing cycle.

In one embodiment, the anti-CD20 antibody is obinutuzumab and/or rituximab.

In one embodiment, the first dosing cycle comprises a first dose (C1D1) of obinutuzumab and a second dose (C1D2) of obinutuzumab. In one embodiment, (a) the subject's body weight is greater than or equal to about 7.5 kg and less than about 13 kg, and wherein the sum of the C1D1 and the C1D2 of obinutuzumab is about 38 mg/kg (e.g., 38 mg/kg±0.25 mg/kg, ±0.5 mg/kg, ±1 mg/kg, ±2 mg/kg, ±3 mg/kg, or ±3.8 mg/kg); (b) the subject's body weight is greater than or equal to about 13 kg and less than about 20 kg, and wherein the sum of the C1D1 and the C1D2 of obinutuzumab is about 28 mg/kg (e.g., 28 mg/kg±0.25 mg/kg, ±0.5 mg/kg, ±1 mg/kg, ±2 mg/kg, or ±2.8 mg/kg); (c) the subject's body weight is greater than or equal to about 20 kg and less than about 32 kg, and wherein the sum of the C1D1 and the C1D2 of obinutuzumab is about 23 mg/kg (e.g., 23 mg/kg±0.25 mg/kg, ±0.5 mg/kg, ±1 mg/kg, ±2 mg/kg, or ±2.3 mg/kg); (d) the subject's body weight is greater than or equal to about 32 kg and less than about 45 kg, and wherein the sum of the C1D1 and the C1D2 of obinutuzumab is about 20 mg/kg (e.g., 23 mg/kg±0.25 mg/kg, ±0.5 mg/kg, ±1 mg/kg, or ±2 mg/kg); or (e) the subject's body weight is greater than or equal to about 45 kg, and wherein the sum of the C1D1 and the C1D2 of obinutuzumab is about 1000 mg (e.g., 1000 mg±5 mg, ±10 mg, ±20 mg, ±30 mg, ±50 mg, ±75 mg, or ±100 mg).

In one embodiment, the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab. In one embodiment, (a) the subject's body weight is greater than or equal to about 7.5 kg and less than about 13 kg, and wherein the C1D1 of obinutuzumab is about 3.8 mg/kg (e.g., 3.8 mg/kg±0.05 mg/kg, 0.1 mg/kg, ±0.2 mg/kg, ±0.3 mg/kg, or ±0.38 mg/kg) and the C1D2 of obinutuzumab is about 34.2 mg/kg (e.g., 34.2 mg/kg±0.5 mg/kg, 1 mg/kg, ±2 mg/kg, ±3 mg/kg, or ±3.42 mg/kg); (b) the subject's body weight is greater than or equal to about 13 kg and less than about 20 kg, and wherein the C1D1 of obinutuzumab is about 2.8 mg/kg (e.g., 2.8 mg/kg±0.05 mg/kg, 0.1 mg/kg, ±0.2 mg/kg, or ±0.28 mg/kg) and the C1D2 of obinutuzumab is about 35.2 mg/kg (e.g., 35.2 mg/kg±0.5 mg/kg, 1 mg/kg, ±2 mg/kg, ±3 mg/kg, or ±3.52 mg/kg); (c) the subject's body weight is greater than or equal to about 20 kg and less than about 32 kg, and wherein the C1D1 of obinutuzumab is about 2.3 mg/kg (e.g., 2.3 mg/kg±0.05 mg/kg, 0.1 mg/kg, ±0.2 mg/kg, or ±0.23 mg/kg) and the C1D2 of obinutuzumab is about 35.7 mg/kg (e.g., 35.7 mg/kg±0.5 mg/kg, 1 mg/kg, ±2 mg/kg, ±3 mg/kg, or ±3.57 mg/kg); (d) the subject's body weight is greater than or equal to about 32 kg and less than about 45 kg, and wherein the C1D1 of obinutuzumab is about 2.0 mg/kg (e.g., 2.0 mg/kg±0.05 mg/kg, 0.1 mg/kg, or ±0.2 mg/kg) and the C1D2 of obinutuzumab is about 36.0 mg/kg (e.g., 36.0 mg/kg±0.5 mg/kg, 1 mg/kg, ±2 mg/kg, ±3 mg/kg, or ±3.6 mg/kg); or (e) the subject's body weight is greater than or equal to about 45 kg, and wherein the C1D1 of obinutuzumab is about 100 mg (e.g., 100 mg±0.5 mg, ±1 mg, ±1.5 mg, ±2 mg, ±4 mg, ±6 mg, ±8 mg, or ±10 mg) and the C1D2 of obinutuzumab is about 900 mg (e.g., 900 mg±5 mg, ±10 mg, ±20 mg, ±30 mg, ±40 mg, ±50 mg, ±60 mg, ±70 mg, ±80 mg, or ±90 mg).

In one embodiment, the C1D1 of obinutuzumab is administered to the subject on Day 1 of the first dosing cycle and the C1D2 of obinutuzumab is administered to the subject on Day 2 of the first dosing cycle.

In one embodiment, the second dosing cycle comprises a single dose (C2D1) of rituximab. In one embodiment, the C2D1 of rituximab is about 375 mg/m² (e.g., 375 mg/m²±5 mg/m², ±10 mg/m², ±25 mg/m², or ±37.5 mg/m²). In one embodiment, rituximab is administered to the subject on Day 5 of the second dosing cycle.

In one embodiment, the method comprises administering to the subject ifosfamide, carboplatin, and etoposide.

In one embodiment, the first dosing cycle comprises:
(a) a first dose (C1D1) of ifosfamide, a second dose (C1D2) of ifosfamide, and a third dose (C1D3) of ifosfamide;
(b) a single dose (C1D1) of carboplatin; and
(c) a first dose (C1D1) of etoposide, a second dose (C1D2) of etoposide, and a third dose (C1D3) of etoposide;
and the second cycle comprises:
(a) a first dose (C2D1) of ifosfamide, a second dose (C2D2) of ifosfamide, and a third dose (C2D3) of ifosfamide;
(b) a single dose (C2D1) of carboplatin; and
(c) a first dose (C2D1) of etoposide, a second dose (C2D2) of etoposide, and a third dose (C2D3) of etoposide.

In one embodiment, ifosfamide is administered at a dose of about 3000 mg/m² (e.g., 3000 mg/m²±40 mg/m², ±50 mg/m², ±100 mg/m², ±200 mg/m², or ±300 mg/m²) for each dose of ifosfamide, carboplatin is administered at a dose of about 635 mg/m² (e.g., 635 mg/m²±5 mg/m², ±10 mg/m², ±25 mg/m², ±50 mg/m², ±60 mg/m², or ±63.5 mg/m²), and etoposide is administered at a dose of about 100 mg/m² (e.g., 100 mg/m²±1 mg/m², ±2.5 mg/m², ±5 mg/m², or ±10 mg/m²) for each dose of etoposide. In one embodiment, (a) the C1D1, C1D2, and C1D3 of ifosfamide are administered on Days 3, 4, and 5, respectively of the first dosing cycle; (b) the C1D1 of carboplatin is administered on Day 3 of the first dosing cycle; (c) the C1D1, C1D2, and C1D3 of etoposide are administered on Days 3, 4, and 5, respectively, of the first dosing cycle; (d) the C2D1, C2D2, and C2D3 of ifosfamide are administered on Days 6, 7, and 8, respectively, of the second dosing cycle; (e) the C2D1 of carboplatin is administered on Day 6 of the second dosing cycle; and (f) the C2D1, C2D2, and C2D3 of etoposide are administered on Days 6, 7, and 8, respectively, of the second dosing cycle.

In one embodiment, the first and second dosing cycles are each 21-day dosing cycles. In one embodiment, the dosing regimen comprises one or more additional dosing cycles. In one embodiment, the one or more additional dosing cycles are each 21-day dosing cycles. In one embodiment, the dosing regimen comprises three dosing cycles in total.

In one embodiment, the one or more additional dosing cycles each comprises:
(a) an additional single dose of the bispecific antibody that binds to CD20 and CD3,
(b) an additional single dose of the anti-CD20 antibody, and
(c) an additional first dose, an additional second dose, and an additional third dose of ifosfamide; an additional single dose of carboplatin; and an additional first dose, an additional second dose, and an additional third dose of etoposide. In one embodiment, (a) the subject's body weight is greater than or equal to about 7.5 kg and less than about 13 kg, and wherein the additional single dose of the bispecific antibody is about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg); (b) the subject's body weight is greater than or equal to about 13 kg and less than about 45 kg, and wherein the additional single dose of the bispecific antibody is about 0.4 mg/kg (e.g., 0.4 mg/kg±0.005 mg/kg, ±0.01 mg/kg, ±0.02 mg/kg, ±0.03 mg/kg, or ±0.04 mg/kg); or (c) the subject's body weight is greater than or equal to about 45 kg, and wherein the additional single dose of the bispecific antibody is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one embodiment, the additional single dose of the bispecific antibody is administered to the subject on Day 1 of each of the one or more additional dosing cycles.

In one embodiment, the anti-CD20 antibody is rituximab. In one embodiment, the additional single dose of rituximab is about 375 mg/m² (e.g., 375 mg/m²±5 mg/m², ±10 mg/m², ±25 mg/m², or ±37.5 mg/m²). In one embodiment, the additional single dose of rituximab is administered on Day 5 of each of the one or more additional dosing cycles.

In one embodiment, the additional first dose, additional second dose, and additional third dose of ifosfamide are each about 3000 mg/m² (e.g., 3000 mg/m²±40 mg/m², ±50 mg/m², ±100 mg/m², ±200 mg/m², or ±300 mg/m²), the additional single dose of carboplatin is about 635 mg/m² (e.g., 635 mg/m²±5 mg/m², ±10 mg/m², ±25 mg/m², ±50 mg/m², ±60 mg/m², or ±63.5 mg/m²), and the additional first dose, the additional second dose, and the additional third dose of etoposide are each about 100 mg/m² (e.g., 100 mg/m²±1 mg/m², ±2.5 mg/m², ±5 mg/m², or ±10 mg/m²). In one embodiment, (a) the additional first dose, the additional second dose, and the additional third dose of ifosfamide are administered to the subject on Days 6, 7, and 8, respectively, of each of the one or more additional dosing cycles; (b) the additional single dose of carboplatin is administered on Day 6 of each of the one or more additional dosing cycles; and (c) the additional first dose, the additional second dose, and the additional third dose of etoposide are administered to the subject on Days 6, 7, and 8, respectively, of each of the one or more additional dosing cycles.

In one embodiment, the method further comprises administering to the subject one or more additional therapeutic agents.

In one embodiment, the one or more additional therapeutic agent is tocilizumab. In one embodiment, the weight of the subject is greater than or equal to about 30 kg and tocilizumab is administered at a dose of about 8 mg/kg (e.g., 8 mg/kg±0.05 mg/kg, ±0.1 mg/kg, ±0.25 mg/kg, ±0.5 mg/kg, or ±0.8 mg/kg) or the weight of the subject is less than 30 kg and tocilizumab is administered at a dose of about 12 mg/kg (e.g., 12 mg/kg±0.05 mg/kg, ±0.1 mg/kg, ±0.25 mg/kg, ±0.5 mg/kg, ±0.75 mg/kg, ±1 mg/kg, or ±1.2 mg/kg), and wherein the maximum dose is about 800 mg (e.g., 800 mg±10 mg, ±25 mg, ±50 mg, or ±80 mg).

In one embodiment, the one or more additional therapeutic agents is a corticosteroid. In one embodiment, the corticosteroid comprises prednisone, prednisolone, methylprednisolone, or dexamethasone.

In one embodiment, the corticosteroid is dexamethasone. In one embodiment, dexamethasone is administered intravenously at a dose of between about 0.15 mg/kg (e.g., 0.15 mg/kg±0.001 mg/kg, ±0.0025 mg/kg, ±0.005 mg/kg, ±0.01 mg/kg, or ±0.015 mg/kg) to about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the bispecific antibody, and wherein the maximum daily dose is 10 mg. In one embodiment, dexamethasone is administered intravenously at a dose of between about 0.15 mg/kg (e.g., 0.15 mg/kg±0.001 mg/kg, ±0.0025 mg/kg, ±0.005 mg/kg, ±0.01 mg/kg, or ±0.015 mg/kg) to about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of obinutuzumab, and wherein the maximum daily dose is 10 mg.

In one embodiment, the corticosteroid is methylprednisolone. In one embodiment, methylprednisolone is administered intravenously at a dose of between about 1 mg/kg to about 2 mg/kg (e.g., 1, 1.1, 1.2, 1.3, 1.4, 1.5, 1.6, 1.7, 1.9, or 2.0 mg/kg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the bispecific antibody. In one embodiment, methylprednisolone is administered intravenously at a dose of between about 1 mg/kg to about 2 mg/kg (e.g., 1, 1.1, 1.2, 1.3, 1.4, 1.5, 1.6, 1.7, 1.9, or 2.0 mg/kg at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of obinutuzumab.

In one embodiment, the corticosteroid is prednisone or prednisolone. In one embodiment, prednisone or prednisolone is administered intravenously at a dose of about 100 mg (e.g., 100 mg±0.5 mg, ±1 mg, ±1.5 mg, ±2 mg, ±4 mg, ±6 mg, ±8 mg, or ±10 mg) or about 2 mg/kg at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the bispecific antibody. In one embodiment, prednisone or prednisolone is administered intravenously at a dose of about 100 mg (e.g., 100 mg±0.5 mg, ±1 mg, ±1.5 mg, ±2 mg, ±4 mg, ±6 mg, ±8 mg, or ±10 mg) or about 2 mg/kg at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of obinutuzumab.

In one embodiment, the one or more additional therapeutic agents is an antihistamine. In one embodiment, the antihistamine is diphenhydramine. In one embodiment, the subject is aged between two years and 17 years, and wherein diphenhydramine is administered intravenously at a dose of between about 10 mg to 20 mg (e.g., 10, 11, 12, 13, 14, 15, 16, 17, 18, 19, or 20 mg) with a maximum single dose of about 1.25 mg/kg. In one embodiment, the subject is aged less than two years, and wherein diphenhydramine is administered rectally at a dose of about 20 mg (e.g., 20 mg±0.1 mg, ±0.25 mg, ±0.5 mg, ±1 mg, ±1.5 mg, or ±2 mg). In one embodiment, diphenhydramine is administered at least about 30 minutes (i.e., at least 30 minutes±3 minutes; e.g., at least about 1, 2, 3, 4, 5, 6, 7, 8, 9, 10, 11, 12, 18, 24, 36, 48 hours, or more) prior to the administration of any dose of the bispecific antibody and/or the anti-CD20 antibody.

In one embodiment, the one or more additional therapeutic agents comprises granulocyte-colony stimulating factor (G-CSF). In one embodiment, G-CSF is administered between about one day and about two days (e.g., 24, 26, 28, 30, 32, 36, 38, 40, 42, 44, 46, or 48 hours) after administration of any dose of rituximab, ifosfamide, carboplatin, and/or etoposide. In one embodiment, G-CSF is administered intravenously or subcutaneously at a dose of about 5 μg/kg/day (e.g., 5 μg/kg/day±0.05 μg/kg/day, ±0.1 μg/kg/day, ±0.2 μg/kg/day, ±0.3 μg/kg/day, ±0.4 μg/kg/day, ±0.5 μg/kg/day), or about 10 μg/kg/day (e.g., 10 μg/kg/day±0.1 μg/kg/day, ±0.2 μg/kg/day, ±0.4 μg/kg/day, ±0.6 μg/kg/day, ±0.8 μg/kg/day, ±1 μg/kg/day). In one embodiment, G-CSF is administered at a dose of about 5 μg/kg/day (e.g., 5 μg/kg/day±0.05 μg/kg/day, ±0.1 μg/kg/day, ±0.2 μg/kg/day, ±0.3 μg/kg/day, ±0.4 μg/kg/day, ±0.5 μg/kg/day) in the first dosing cycle and about 10 μg/kg/day (e.g., 10 μg/kg/day±0.1 μg/kg/day, ±0.2 μg/kg/day, ±0.4 μg/kg/day, ±0.6 μg/kg/day, ±0.8 μg/kg/day, ±1 μg/kg/day) in the second dosing cycle and/or each additional dosing cycle.

In one embodiment, the one or more additional therapeutic agents is an antipyretic. In one embodiment, the antipyretic is acetaminophen or paracetamol. In one embodiment, acetaminophen or paracetamol is administered orally or intravenously at a dose of between about 500 to about 1000 mg (e.g., 500, 550, 600, 650, 700, 750, 800, 850, 900, 950, or 1000 mg). In one embodiment, acetaminophen or paracetamol is administered at least about 30 minutes (i.e., at least 30 minutes±3 minutes; e.g., at least about 1, 2, 3, 4, 5, 6, 7, 8, 9, 10, 11, 12, 18, 24, 36, 48 hours, or more) prior to the administration of any dose of the bispecific antibody and/or the anti-CD20 antibody.

In one embodiment, the one or more additional therapeutic agents is mesna. In one embodiment, mesna is administered intravenously daily as five doses totaling 3000 mg/m² in amount. In one embodiment, mesna is administered intravenously at a first dose of about 600 mg/m² prior to the administration of any dose of ifosfamide and at four repeated doses of about 600 mg/m² each at about three hours, about six hours, about nine hours, and about 12 hours, respectively, after the first dose of ifosfamide. In one embodiment, mesna is administered daily to the subject on Days 3, 4, and 5 of the first dosing cycle, on Days 6, 7, and 8 of the second dosing cycle, and/or on Days 6, 7, and 8 of each additional dosing cycle.

In one aspect, the invention features a method of treating a subject aged between 18 years and 30 years having a CD20-positive cell proliferative disorder comprising administering to the subject an effective amount of
(a) a bispecific antibody that binds to CD20 and CD3;
(b) an anti-CD20 antibody; and
(c) one or more chemotherapeutic agents selected from ifosfamide, carboplatin, and/or etoposide in a dosing regimen comprising at least a first dosing cycle and a second dosing cycle.

In one embodiment, the first dosing cycle comprises a first dose (C1D1) of the bispecific antibody and a second dose (C1D2) of the bispecific antibody, wherein the C1D1 of the bispecific antibody is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of the bispecific antibody is about 10 mg (e.g., 10 mg±0.05 mg, ±0.1 mg, ±0.2 mg, ±0.3 mg, ±0.5 mg, ±0.75 mg, or ±1 mg); and the second dosing cycle comprises a single dose (C2D1) of the bispecific antibody, wherein the C2D1 of the bispecific antibody is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one embodiment, the C1D1 of the bispecific antibody and the C1D2 of the bispecific antibody are administered to the subject on Days 8 and 15, respectively, of the first dosing cycle.

In one embodiment, the C2D1 of the bispecific antibody is administered to the subject on Day 1 of the second dosing cycle.

In one embodiment, the anti-CD20 antibody is obinutuzumab and/or rituximab.

In one embodiment, the first dosing cycle comprises a first dose (C1D1) of obinutuzumab and a second dose (C1D2) of obinutuzumab. In one embodiment, the sum of the C1D1 and the C1D2 of obinutuzumab is about 1000 mg (e.g., 1000 mg±5 mg, ±10 mg, ±20 mg, ±30 mg, ±50 mg, ±75 mg, or ±100 mg). In one embodiment, the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab. In one embodiment, the C1D1 of obinutuzumab is about 100 mg (e.g., 100 mg±0.5 mg, ±1 mg, ±1.5 mg, ±2 mg, ±4 mg, ±6 mg, ±8 mg, or ±10 mg) and the C1D2 of obinutuzumab is about 900 mg (e.g., 900 mg±5 mg, ±10 mg, ±20 mg, ±30 mg, ±40 mg, ±50 mg, ±60 mg, ±70 mg, ±80 mg, or ±90 mg). In one embodiment, the C1D1 of obinutuzumab is administered to the subject on Day 1 of the first dosing cycle and the C1D2 of obinutuzumab is administered to the subject on Day 2 of the first dosing cycle.

In one embodiment, the second dosing cycle comprises a single dose (C2D1) of rituximab. In one embodiment, the C2D1 of rituximab is about 375 mg/m² (e.g., 375 mg/m²±5 mg/m², ±10 mg/m², ±25 mg/m², or ±37.5 mg/m²). In one embodiment, the C2D1 of rituximab is administered to the subject on Day 5 of the second dosing cycle.

In one embodiment, the method comprises administering to the subject ifosfamide, carboplatin, and etoposide. In one embodiment, the first dosing cycle comprises:
(a) a single dose (C1D1) of ifosfamide;
(b) a single dose (C1D1) of carboplatin; and
(c) a first dose (C1D1) of etoposide, a second dose (C1D2) of etoposide, and a third dose (C1D3) of etoposide;
and the second cycle comprises:
(a) a single dose (C2D1) of ifosfamide;
(b) a single dose (C2D1) of carboplatin; and
(c) a first dose (C2D1) of etoposide, a second dose (C2D2) of etoposide, and a third dose (C2D3) of etoposide.

In one embodiment, ifosfamide is administered at a dose of about 5000 mg/m² (e.g., 5000 mg/m²±50 mg/m², ±100 mg/m², ±200 mg/m², ±300 mg/m², ±400 mg/m², or ±500 mg/m²), carboplatin is administered at a dose of about 5×(25+CreatinineClearance (CrCl)) mg with maximum dose of about 750 mg (e.g., 750 mg±10 mg, ±25 mg, ±50 mg, or ±75 mg), and etoposide is administered at a dose of about 100 mg/m² (e.g., 100 mg/m²±1 mg/m², ±2.5 mg/m², ±5 mg/m², or ±10 mg/m²) for each dose of etoposide.

In one embodiment, (a) the subject is male, and CrCl is calculated using the formula CrCl=([140−age]×[weight in kg])/(72×[serum creatinine in mg/dL]); or (b) the subject is female, and CrCl is calculated using the formula CrCl=0.85×([140−age]×[weight in kg])/(72×[serum creatinine in mg/dL]).

In one embodiment, (a) the subject has CrCl<about 60 mL/min, and each single dose of ifosfamide is reduced to 4000 mg/m²; and/or (b) the subject has CrCl<about 50 mL/min, and each dose of etoposide is reduced to about 75 mg/m².

In one embodiment, (a) the C1D1 ifosfamide is administered on Day 3 of the first dosing cycle;
(b) the C1D1 of carboplatin is administered on Day 3 of the first dosing cycle;
(c) the C1D1, C1D2, and C1D3 of etoposide are administered on Days 3, 4, and 5, respectively, of the first dosing cycle;
(d) the C2D1 of ifosfamide is administered on Day 6 of the second dosing cycle;
(e) the C2D1 of carboplatin is administered on Day 6 of the second dosing cycle; and
(f) the C2D1, C2D2, and C2D3 of etoposide are administered on Days 6, 7, and 8, respectively, of the second dosing cycle.

In one embodiment, the first and second dosing cycles are each 21-day dosing cycles. In one embodiment, the dosing regimen comprises one or more additional dosing cycles. In one embodiment, the one or more additional dosing cycles are each 21-day dosing cycles. In one embodiment, the dosing regimen comprises three dosing cycles in total.

In one embodiment, the one or more additional dosing cycles each comprise:
(a) an additional single dose of the bispecific antibody that binds to CD20 and CD3,
(b) an additional single dose of the anti-CD20 antibody, and
(c) an additional single dose of ifosfamide; an additional single dose of carboplatin; and an additional first dose, an additional second dose, and an additional third dose of etoposide.

In one embodiment, the additional single dose of the bispecific antibody is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg). In one embodiment, the additional single dose of the bispecific antibody is administered to the subject on Day 1 of each of the one or more additional dosing cycles.

In one embodiment, the anti-CD20 antibody is rituximab. In one embodiment, the additional single dose of rituximab is about 375 mg/m² (e.g., 375 mg/m²±5 mg/m², ±10 mg/m², ±25 mg/m², or ±37.5 mg/m²). In one embodiment, the additional single dose of rituximab is administered on Day 5 of each of the one or more additional dosing cycles.

In one embodiment, the additional single dose of ifosfamide is about 5000 mg/m² (e.g., 5000 mg/m²±50 mg/m², ±100 mg/m², ±200 mg/m², ±300 mg/m², ±400 mg/m², or ±500 mg/m²), the additional single dose of carboplatin is about 5×(25+CreatinineClearance (CrCl)) mg with maximum dose of about 750 mg (e.g., 750 mg±10 mg, ±25 mg, ±50 mg, or ±75 mg), and the additional first dose, the additional second dose, and the additional third dose of etoposide are each about 100 mg/m² (e.g., 100 mg/m²±1 mg/m², ±2.5 mg/m², ±5 mg/m², or ±10 mg/m²).

In one embodiment, (a) the subject is male, and wherein CrCl is calculated using the formula CrCl=([140−age]×[weight in kg])/(72×[serum creatinine in mg/dL]); or (b) the subject is female, and wherein CrCl is calculated using the formula CrCl=0.85×([140−age]×[weight in kg])/(72×[serum creatinine in mg/dL]).

In one embodiment, (a) the subject has CrCl<about 60 mL/min, and the additional single dose of ifosfamide is reduced to 4000 mg/m²; and/or (b) the subject has CrCl<about 50 mL/min, and each additional dose of etoposide is reduced to about 75 mg/m².

In one embodiment, (a) the additional single dose of ifosfamide is administered on Day 6 of each of the one or more additional dosing cycles;
(b) the additional single dose of carboplatin is administered on Day 6 of each of the one or more additional dosing cycles; and
(c) the additional first dose, the additional second dose, and the additional third dose of etoposide are administered to the subject on Days 6, 7, and 8, respectively, of each of the one or more additional dosing cycles.

In one embodiment, the method further comprises administering to the subject one or more additional therapeutic agents.

In one embodiment, the one or more additional therapeutic agent is tocilizumab. In one embodiment, the weight of the subject is greater than or equal to about 30 kg and tocilizumab is administered at a dose of about 8 mg/kg (e.g., 8 mg/kg±0.05 mg/kg, ±0.1 mg/kg, ±0.25 mg/kg, ±0.5 mg/kg, or ±0.8 mg/kg) or the weight of the subject is less than 30 kg and tocilizumab is administered at a dose of about 12 mg/kg (e.g., 12 mg/kg±0.05 mg/kg, ±0.1 mg/kg, ±0.25 mg/kg, ±0.5 mg/kg, ±0.75 mg/kg, ±1 mg/kg, or ±1.2 mg/kg), and wherein the maximum dose is about 800 mg (e.g., 800 mg±10 mg, ±25 mg, ±50 mg, or ±80 mg).

In one embodiment, the one or more additional therapeutic agents is a corticosteroid.

In one embodiment, the corticosteroid comprises prednisone, prednisolone, methylprednisolone, or dexamethasone.

In one embodiment, the corticosteroid is dexamethasone. In one embodiment, dexamethasone is administered intravenously at a dose of between about 0.15 mg/kg (e.g., 0.15 mg/kg±0.001 mg/kg, ±0.0025 mg/kg, ±0.005 mg/kg, ±0.01 mg/kg, or ±0.015 mg/kg) to about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the bispecific antibody, and wherein the maximum daily dose is 10 mg. In one embodiment, dexamethasone is administered intravenously at a dose of between about 0.15 mg/kg (e.g., 0.15 mg/kg±0.001 mg/kg, ±0.0025 mg/kg, ±0.005 mg/kg, ±0.01 mg/kg, or ±0.015 mg/kg) to about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of obinutuzumab, and wherein the maximum daily dose is 10 mg.

In one embodiment, the corticosteroid is methylprednisolone. In one embodiment, methylprednisolone is administered intravenously at a dose of between about 1 mg/kg to about 2 mg/kg (e.g., 1, 1.1, 1.2, 1.3, 1.4, 1.5, 1.6, 1.7, 1.9, or 2.0 mg/kg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the bispecific antibody. In one embodiment, methylprednisolone is administered intravenously at a dose of between about 1 mg/kg to about 2 mg/kg (e.g., 1, 1.1, 1.2, 1.3, 1.4, 1.5, 1.6, 1.7, 1.9, or 2.0 mg/kg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of obinutuzumab.

In one embodiment, the corticosteroid is prednisone or prednisolone. In one embodiment, prednisone or prednisolone is administered intravenously at a dose of about 100 mg (e.g., 100 mg±0.5 mg, ±1 mg, ±1.5 mg, ±2 mg, ±4 mg, ±6 mg, ±8 mg, or ±10 mg) or about 2 mg/kg at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the bispecific antibody. In one embodiment, prednisone or prednisolone is administered intravenously at a dose of about 100 mg (e.g., 100 mg±0.5 mg, ±1 mg, ±1.5 mg, ±2 mg, ±4 mg, ±6 mg, ±8 mg, or ±10 mg) or about 2 mg/kg at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of obinutuzumab.

In one embodiment, the one or more additional therapeutic agents is an antihistamine. In one embodiment, the antihistamine is diphenhydramine. In one embodiment, diphenhydramine is administered orally or intravenously at a dose of about 50 mg (e.g., 50 mg±0.5 mg, ±1 mg, ±1.5 mg, ±2 mg, ±3 mg, ±4 mg, or ±5 mg). In one embodiment, diphenhydramine is administered at least about 30 minutes (i.e., at least 30 minutes±3 minutes; e.g., at least about 1, 2, 3, 4, 5, 6, 7, 8, 9, 10, 11, 12, 18, 24, 36, 48 hours, or more) prior to the administration of any dose of the bispecific antibody and/or the anti-CD20 antibody.

In one embodiment, the one or more additional therapeutic agents comprises granulocyte-colony stimulating factor (G-CSF). In one embodiment, G-CSF is administered between about one day and about two days (e.g., 24, 26, 28, 30, 32, 36, 38, 40, 42, 44, 46, or 48 hours) after administration of any dose of rituximab, ifosfamide, carboplatin, and/or etoposide. In one embodiment, G-CSF is administered intravenously or subcutaneously at a dose of about 5 μg/kg/day (e.g., 5 μg/kg/day±0.05 μg/kg/day, ±0.1 μg/kg/day, ±0.2 μg/kg/day, ±0.3 μg/kg/day, ±0.4 μg/kg/day, ±0.5 μg/kg/day) or about 10 μg/kg/day (e.g., 10 μg/kg/day±0.1 μg/kg/day, ±0.2 μg/kg/day, ±0.4 μg/kg/day, ±0.6 μg/kg/day, ±0.8 μg/kg/day, ±1 μg/kg/day). In one embodiment, G-CSF is administered at a dose of about 5 μg/kg/day (e.g., 5 μg/kg/day±0.05 μg/kg/day, ±0.1 μg/kg/day, ±0.2 μg/kg/day, ±0.3 μg/kg/day, ±0.4 μg/kg/day, ±0.5 μg/kg/day) in the first dosing cycle and about 10 μg/kg/day (e.g., 10 μg/kg/day±0.1 μg/kg/day, ±0.2 μg/kg/day, ±0.4 μg/kg/day, ±0.6 μg/kg/day, ±0.8 μg/kg/day, ±1 μg/kg/day) in the second dosing cycle and/or each additional dosing cycle.

In one embodiment, the one or more additional therapeutic agents is an antipyretic. In one embodiment, the antipyretic is acetaminophen or paracetamol. In one embodiment, acetaminophen or paracetamol is administered orally or intravenously at a dose of between about 500 to about 1000 mg (e.g., 500, 550, 600, 650, 700, 750, 800, 850, 900, 950, or 1000 mg). In one embodiment, acetaminophen or paracetamol is administered at least about 30 minutes (i.e., at least 30 minutes±3 minutes; e.g., at least about 1, 2, 3, 4, 5, 6, 7, 8, 9, 10, 11, 12, 18, 24, 36, 48 hours, or more) prior to the administration of any dose of the bispecific antibody and/or the anti-CD20 antibody.

In one embodiment, the one or more additional therapeutic agents is mesna. In one embodiment, mesna is administered intravenously at a dose of about 5000 mg/m² (e.g., 5000 mg/m²±50 mg/m², ±100 mg/m², ±200 mg/m², ±300 mg/m², ±400 mg/m², or ±500 mg/m²). In one embodiment, mesna is administered via continuous infusion over about 24 hours on Day 3 of the first dosing cycle, on Day 6 of the second dosing cycle, and/or on Day 6 of each additional dosing cycle. In one embodiment, mesna is administered simultaneously with any dose of ifosfamide.

In one aspect, the invention features a method of treating a subject having a CD20-positive cell proliferative disorder comprising administering to the subject an effective amount of glofitamab, obinutuzumab, rituximab, ifosfamide, carboplatin, and etoposide in a dosing regimen comprising at least a first dosing cycle and a second dosing cycle, wherein
(a) the first dosing cycle comprises administering a first dose (C1D1) of glofitamab on Day 8 and administering a second dose (C1D2) of glofitamab on Day 15, wherein the C1D1 of glofitamab is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 10 mg (e.g., 10 mg±0.05 mg, ±0.1 mg, ±0.2 mg, ±0.3 mg, ±0.5 mg, ±0.75 mg, or ±1 mg); and
(b) the second dosing cycle comprises administering a single dose (C2D1) of glofitamab on Day 8, wherein the C2D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one aspect, the invention features a method of treating a subject having a CD20-positive cell proliferative disorder comprising administering to the subject an effective amount of glofitamab, obinutuzumab, rituximab, ifosfamide, carboplatin, and etoposide in a dosing regimen comprising a first dosing cycle, a second dosing cycle, and a third dosing cycle, wherein
(a) the first dosing cycle comprises administering a first dose (C1D1) of glofitamab on Day 8 and administering a second dose (C1D2) of glofitamab on Day 15, wherein the C1D1 of glofitamab is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 10 mg (e.g., 10 mg±0.05 mg, ±0.1 mg, ±0.2 mg, ±0.3 mg, ±0.5 mg, ±0.75 mg, or ±1 mg);
(b) the second dosing cycle comprises administering a single dose (C2D1) of glofitamab on Day 8, wherein the C2D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg); and
(c) the third dosing cycle comprises administering a single dose (C3D1) of glofitamab on Day 8, wherein the C3D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one aspect, the invention features a method of treating a subject having a CD20-positive cell proliferative disorder comprising administering to the subject an effective amount of glofitamab, obinutuzumab, rituximab, ifosfamide, carboplatin, and etoposide in a dosing regimen comprising at least a first dosing cycle and a second dosing cycle, wherein
(a) the first dosing cycle comprises:
   (i) administering a first dose (C1D1) of glofitamab on Day 8 and a second dose (C1D2) of glofitamab on Day 15, wherein the C1D1 of glofitamab is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 10 mg (e.g., 10 mg±0.05 mg, ±0.1 mg, ±0.2 mg, ±0.3 mg, ±0.5 mg, ±0.75 mg, or ±1 mg);
   (ii) administering a first dose (C1D1) of obinutuzumab on Day 1, wherein the C1D1 of obinutuzumab is about 1000 mg (e.g., 1000 mg±5 mg, ±10 mg, ±20 mg, ±30 mg, ±50 mg, ±75 mg, or ±100 mg);
   (iii) administering a single dose (C1D1) of ifosfamide on Day 2, wherein the C1D1 of ifosfamide is about 5000 mg/m² (e.g., 5000 mg/m²±50 mg/m², ±100 mg/m², ±200 mg/m², ±300 mg/m², ±400 mg/m², or ±500 mg/m²) with a maximum dose of about 800 mg (e.g., 800 mg±10 mg, ±25 mg, ±50 mg, or ±80 mg);
   (iv) administering a single dose (C1D1) of carboplatin on Day 2, wherein the C1D1 of carboplatin is about 5×(25+creatinine clearance) mg; and
   (v) administering a first dose (C1D1) of etoposide on Day 1, a second dose (C1D2) of etoposide on Day 2, and a third dose (C1D3) of etoposide on Day 3, wherein the C1D1, the C1D2, and the C1D3 of etoposide are each about 100 mg/m² (e.g., 100 mg/m²±1 mg/m², ±2.5 mg/m², ±5 mg/m², or ±10 mg/m²); and
(b) the second dosing cycle comprises:
   (i) administering a single dose (C2D1) of glofitamab on Day 8, wherein the C2D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg);
   (ii) administering a first dose (C2D1) of rituximab on Day 1, wherein the C2D1 of rituximab is about 375 mg/m² (e.g., 375 mg/m²±5 mg/m², ±10 mg/m², ±25 mg/m², or ±37.5 mg/m²);
   (iii) administering a single dose (C2D1) of ifosfamide on Day 2, wherein the C2D1 of ifosfamide is about 5000 mg/m² (e.g., 5000 mg/m²±50 mg/m², ±100 mg/m², ±200 mg/m², ±300 mg/m², ±400 mg/m², or ±500 mg/m²) with a maximum dose of about 800 mg (e.g., 800 mg±10 mg, ±25 mg, ±50 mg, or ±80 mg);
   (iv) administering a single dose (C2D1) of carboplatin on Day 2, wherein the C2D1 of carboplatin is about 5×(25+creatinine clearance) mg; and
(v) administering a first dose (C2D1) of etoposide on Day 1, a second dose (C2D2) of etoposide on Day 2, and a third dose (C2D3) of etoposide on Day 3, wherein the C2D1, the C2D2, and the C2D3 of etoposide are each about 100 mg/m² (e.g., 100 mg/m²±1 mg/m², ±2.5 mg/m², ±5 mg/m², or ±10 mg/m²).

In one aspect, the invention features a method of treating a subject having a CD20-positive cell proliferative disorder comprising administering to the subject an effective amount of glofitamab, obinutuzumab, rituximab, ifosfamide, carboplatin, and etoposide in a dosing regimen comprising a first dosing cycle, a second dosing cycle, and a third dosing cycle, wherein
(a) the first dosing cycle comprises:
   (i) administering a first dose (C1D1) of glofitamab on Day 8 and a second dose (C1D2) of glofitamab on Day 15, wherein the C1D1 of glofitamab is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 10 mg (e.g., 10 mg±0.05 mg, ±0.1 mg, ±0.2 mg, ±0.3 mg, ±0.5 mg, ±0.75 mg, or ±1 mg);
   (ii) administering a first dose (C1D1) of obinutuzumab on Day 1, wherein the C1D1 of obinutuzumab is about 1000 mg (e.g., 1000 mg±5 mg, ±10 mg, ±20 mg, ±30 mg, ±50 mg, ±75 mg, or ±100 mg);
   (iii) administering a single dose (C1D1) of ifosfamide on Day 2, wherein the C1D1 of ifosfamide is about 5000 mg/m² (e.g., 5000 mg/m²±50 mg/m², ±100 mg/m², ±200 mg/m², ±300 mg/m², ±400 mg/m², or ±500 mg/m²) with a maximum dose of about 800 mg (e.g., 800 mg±10 mg, ±25 mg, ±50 mg, or ±80 mg);
   (iv) administering a single dose (C1D1) of carboplatin on Day 2, wherein the C1D1 of carboplatin is about 5×(25+creatinine clearance) mg; and
   (v) administering a first dose (C1D1) of etoposide on Day 1, a second dose (C1D2) of etoposide on Day 2, and a third dose (C1D3) of etoposide on Day 3, wherein the C1D1, the C1D2, and the C1D3 of etoposide are each about 100 mg/m² (e.g., 100 mg/m²±1 mg/m², ±2.5 mg/m², ±5 mg/m², or ±10 mg/m²);
(b) the second dosing cycle comprises:
   (i) administering a single dose (C2D1) of glofitamab on Day 8, wherein the C2D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg);
   (ii) administering a first dose (C2D1) of rituximab on Day 1, wherein the C2D1 of rituximab is about 375 mg/m² (e.g., 375 mg/m²±5 mg/m², ±10 mg/m², ±25 mg/m², or ±37.5 mg/m²);
   (iii) administering a single dose (C2D1) of ifosfamide on Day 2, wherein the C2D1 of ifosfamide is about 5000 mg/m² (e.g., 5000 mg/m²±50 mg/m², ±100 mg/m², ±200 mg/m², ±300 mg/m², ±400 mg/m², or ±500 mg/m²) with a maximum dose of about 800 mg (e.g., 800 mg±10 mg, ±25 mg, ±50 mg, or ±80 mg);
   (iv) administering a single dose (C2D1) of carboplatin on Day 2, wherein the C2D1 of carboplatin is about 5×(25+creatinine clearance) mg; and
   (v) administering a first dose (C2D1) of etoposide on Day 1, a second dose (C2D2) of etoposide on Day 2, and a third dose (C2D3) of etoposide on Day 3, wherein the C2D1, the C2D2, and the C2D3 of etoposide are each about 100 mg/m² (e.g., 100 mg/m²±1 mg/m², ±2.5 mg/m², ±5 mg/m², or ±10 mg/m²); and
(c) the third dosing cycle comprises:
   (i) administering a single dose (C3D1) of glofitamab on Day 8, wherein the C3D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg);
   (ii) administering a first dose (C3D1) of rituximab on Day 1, wherein the C3D1 of rituximab is about 375 mg/m² (e.g., 375 mg/m²±5 mg/m², ±10 mg/m², ±25 mg/m², or ±37.5 mg/m²);
   (iii) administering a single dose (C3D1) of ifosfamide on Day 2, wherein the C3D1 of ifosfamide is about 5000 mg/m² (e.g., 5000 mg/m²±50 mg/m², ±100 mg/m², ±200 mg/m², ±300 mg/m², ±400 mg/m², or ±500 mg/m²) with a maximum dose of about 800 mg (e.g., 800 mg±10 mg, ±25 mg, ±50 mg, or ±80 mg);
   (iv) administering a single dose (C3D1) of carboplatin on Day 2, wherein the C3D1 of carboplatin is about 5×(25+creatinine clearance) mg; and
   (v) administering a first dose (C3D1) of etoposide on Day 1, a second dose (C3D2) of etoposide on Day 2, and a third dose (C3D3) of etoposide on Day 3, wherein the C3D1, the C3D2, and the C3D3 of etoposide are each about 100 mg/m² (e.g., 100 mg/m²±1 mg/m², ±2.5 mg/m², ±5 mg/m², or ±10 mg/m²).

In one embodiment, mesna is administered simultaneously with any dose of ifosfamide. In one embodiment, mesna is administered at a dose of about 5000 mg/m² (e.g., 5000 mg/m²±50 mg/m², ±100 mg/m², ±200 mg/m², ±300 mg/m², ±400 mg/m², or ±500 mg/m²) intravenously. In one embodiment, mesna is administered via continuous infusion over about 24 hours on Day 2 of each dosing cycle.

In one aspect, the invention features a method of treating a subject aged between 6 months and 17 years having a CD20-positive cell proliferative disorder comprising administering to the subject an effective amount of glofitamab, obinutuzumab, rituximab, ifosfamide, carboplatin, and etoposide in a dosing regimen comprising at least a first dosing cycle and a second dosing cycle, wherein
(a) the first dosing cycle comprises administering a first dose (C1D1) of glofitamab on Day 8 and administering a second dose (C1D2) of glofitamab on Day 15, wherein the C1D1 of glofitamab is about 0.03 mg/kg (e.g., 0.03 mg/kg±0.0005 mg/kg, ±0.001 mg/kg, ±0.002 mg/kg, or ±0.003 mg/kg), about 0.04 mg/kg (e.g., 0.04 mg/kg±0.0005 mg/kg, ±0.001 mg/kg, ±0.002 mg/kg, ±0.003 mg/kg, or ±0.004 mg/kg), or about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 0.15 mg/kg (e.g., 0.15 mg/kg±0.001 mg/kg, ±0.0025 mg/kg, ±0.005 mg/kg, ±0.01 mg/kg, or ±0.015 mg/kg) or about 10 mg (e.g., 10 mg±0.05 mg, ±0.1 mg, ±0.2 mg, ±0.3 mg, ±0.5 mg, ±0.75 mg, or ±1 mg); and
(b) the second dosing cycle comprises administering a single dose (C2D1) of glofitamab on Day 8, wherein the C2D1 of glofitamab is about 0.4 mg/kg (e.g., 0.4 mg/kg±0.005 mg/kg, ±0.01 mg/kg, ±0.02 mg/kg, ±0.03 mg/kg, or ±0.04 mg/kg), about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg), or about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one aspect, the invention features a method of treating a subject aged between 6 months and 17 years having a CD20-positive cell proliferative disorder comprising administering to the subject an effective amount of glofitamab, obinutuzumab, rituximab, ifosfamide, carboplatin, and etoposide in a dosing regimen comprising a first dosing cycle, a second dosing cycle, and a third dosing cycle, wherein
(a) the first dosing cycle comprises administering a first dose (C1D1) of glofitamab on Day 8 and administering a second dose (C1D2) of glofitamab on Day 15, wherein the C1D1 of glofitamab is about 0.03 mg/kg (e.g., 0.03 mg/kg±0.0005 mg/kg, ±0.001 mg/kg, ±0.002 mg/kg, or ±0.003 mg/kg), about 0.04 mg/kg (e.g., 0.04 mg/kg±0.0005 mg/kg, ±0.001 mg/kg, ±0.002 mg/kg, ±0.003 mg/kg, or ±0.004 mg/kg), or about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 0.15 mg/kg (e.g., 0.15 mg/kg±0.001 mg/kg, ±0.0025 mg/kg, ±0.005 mg/kg, ±0.01 mg/kg, or ±0.015 mg/kg) or about 10 mg (e.g., 10 mg±0.05 mg, ±0.1 mg, ±0.2 mg, ±0.3 mg, ±0.5 mg, ±0.75 mg, or ±1 mg);
(b) the second dosing cycle comprises administering a single dose (C2D1) of glofitamab on Day 8, wherein the C2D1 of glofitamab is about 0.4 mg/kg (e.g., 0.4 mg/kg±0.005 mg/kg, ±0.01 mg/kg, ±0.02 mg/kg, ±0.03 mg/kg, or ±0.04 mg/kg), about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg), or about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg); and
(c) the third dosing cycle comprises administering a single dose (C3D1) of glofitamab on Day 8, wherein the C3D1 of glofitamab is about 0.4 mg/kg (e.g., 0.4 mg/kg±0.005 mg/kg, ±0.01 mg/kg, ±0.02 mg/kg, ±0.03 mg/kg, or ±0.04 mg/kg), about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg), or about 30 mg (e.g., 30 mg±0.1 mg, ±0.2 mg, ±0.3 mg, ±0.5 mg, ±0.75 mg, ±1 mg, ±1.5 mg, ±2 mg, or 3 mg).

In one aspect, the invention features a method of treating a subject aged between 6 months and 17 years having a CD20-positive cell proliferative disorder comprising administering to the subject an effective amount of glofitamab, obinutuzumab, rituximab, ifosfamide, carboplatin, and etoposide in a dosing regimen comprising at least a first dosing cycle and a second dosing cycle, wherein
(a) the first dosing cycle comprises:
   (i) administering a first dose (C1D1) of glofitamab on Day 8 and a second dose (C1D2) of glofitamab on Day 15, wherein the C1D1 of glofitamab is about 0.03 mg/kg (e.g., 0.03 mg/kg±0.0005 mg/kg, ±0.001 mg/kg, ±0.002 mg/kg, or ±0.003 mg/kg), about 0.04 mg/kg (e.g., 0.04 mg/kg±0.0005 mg/kg, ±0.001 mg/kg, ±0.002 mg/kg, ±0.003 mg/kg, or ±0.004 mg/kg), or about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 0.15 mg/kg (e.g., 0.15 mg/kg±0.001 mg/kg, ±0.0025 mg/kg, ±0.005 mg/kg, ±0.01 mg/kg, or ±0.015 mg/kg) or about 10 mg (e.g., 10 mg±0.05 mg, ±0.1 mg, ±0.2 mg, ±0.3 mg, ±0.5 mg, ±0.75 mg, or ±1 mg);
   (ii) administering a first dose (C1D1) of obinutuzumab on Day 1 and a second dose (C1D2) of obinutuzumab on Day 2, wherein the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab, and wherein the sum of the C1D1 and the C1D2 of obinutuzumab is about 38 mg/kg (e.g., 38 mg/kg±0.25 mg/kg, ±0.5 mg/kg, ±1 mg/kg, ±2 mg/kg, ±3 mg/kg, or ±3.8 mg/kg), about 28 mg/kg (e.g., 28 mg/kg±0.25 mg/kg, ±0.5 mg/kg, ±1 mg/kg, ±2 mg/kg, or ±2.8 mg/kg), about 23 mg/kg (e.g., 23 mg/kg±0.25 mg/kg, ±0.5 mg/kg, ±1 mg/kg, ±2 mg/kg, or ±2.3 mg/kg), about 20 mg/kg, or about 1000 mg (e.g., 1000 mg±5 mg, ±10 mg, ±20 mg, ±30 mg, ±50 mg, ±75 mg, or ±100 mg);
   (iii) administering a first dose (C1D1) of ifosfamide on Day 3, a second dose (C1D2) of ifosfamide on Day 4, and a third dose (C1D3) of ifosfamide on Day 5, wherein the C1D1, the C1D2, and the C1D3 of ifosfamide are each about 3000 mg/m² (e.g., 3000 mg/m²±40 mg/m², ±50 mg/m², ±100 mg/m², ±200 mg/m², or ±300 mg/m²);
   (iv) administering a single dose (C1D1) of carboplatin on Day 3, wherein the C1D1 of carboplatin is about 635 mg/m² (e.g., 635 mg/m²±5 mg/m², ±10 mg/m², ±25 mg/m², ±50 mg/m², ±60 mg/m², or ±63.5 mg/m²); and
   (v) administering a first dose (C1D1) of etoposide on Day 3, a second dose (C1D2) of etoposide on Day 4, and a third dose (C1D3) of etoposide on Day 5, wherein the C1D1, the C1D2, and the C1D3 of etoposide are each about 100 mg/m² (e.g., 100 mg/m²±1 mg/m², ±2.5 mg/m², ±5 mg/m², or ±10 mg/m²); and
(b) the second dosing cycle comprises:
   (i) administering a single dose (C2D1) of glofitamab on Day 1, wherein the C2D1 of glofitamab is about 0.4 mg/kg (e.g., 0.4 mg/kg±0.005 mg/kg, ±0.01 mg/kg, ±0.02 mg/kg, ±0.03 mg/kg, or ±0.04 mg/kg), about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg), or about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg);
   (ii) administering a first dose (C2D1) of rituximab on Day 5, wherein the C2D1 of rituximab is about 375 mg/m² (e.g., 375 mg/m²±5 mg/m², ±10 mg/m², ±25 mg/m², or ±37.5 mg/m²);
   (iii) administering a first dose (C2D1) of ifosfamide on Day 6, a second dose (C2D2) of ifosfamide on Day 7, and a third dose (C2D3) of ifosfamide on Day 8, wherein the C2D1, the C2D2, and the C2D3 of ifosfamide are each about 3000 mg/m² (e.g., 3000 mg/m²±40 mg/m², ±50 mg/m², ±100 mg/m², ±200 mg/m², or ±300 mg/m²);
   (iv) administering a single dose (C2D1) of carboplatin on Day 6, wherein the C2D1 of carboplatin is about 5×(25+creatinine clearance) mg; and
   (v) administering a first dose (C2D1) of etoposide on Day 6, a second dose (C2D2) of etoposide on Day 7, and a third dose (C2D3) of etoposide on Day 8, wherein the C2D1, the C2D2, and the C2D3 of etoposide are each about 100 mg/m² (e.g., 100 mg/m²±1 mg/m², ±2.5 mg/m², ±5 mg/m², or ±10 mg/m²).

In one aspect, the invention features a method of treating a subject aged between 6 months and 17 years having a CD20-positive cell proliferative disorder comprising administering to the subject an effective amount of glofitamab, obinutuzumab, rituximab, ifosfamide, carboplatin, and etoposide in a dosing regimen comprising a first dosing cycle, a second dosing cycle, and a third dosing cycle, wherein
(a) the first dosing cycle comprises:
   (i) administering a first dose (C1D1) of glofitamab on Day 8 and a second dose (C1D2) of glofitamab on Day 15, wherein the C1D1 of glofitamab is about 0.03 mg/kg, about 0.04 mg/kg (e.g., 0.04 mg/kg±0.0005 mg/kg, ±0.001 mg/kg, ±0.002 mg/kg, ±0.003 mg/kg, or ±0.004 mg/kg), or about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 0.15 mg/kg (e.g., 0.15 mg/kg±0.001 mg/kg, ±0.0025 mg/kg, ±0.005 mg/kg, ±0.01 mg/kg, or ±0.015 mg/kg) or about 10 mg (e.g., 10 mg±0.05 mg, ±0.1 mg, ±0.2 mg, ±0.3 mg, ±0.5 mg, ±0.75 mg, or ±1 mg);
   (ii) administering a first dose (C1D1) of obinutuzumab on Day 1 and a second dose (C1D2) of obinutuzumab on Day 2, wherein the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab, and wherein the sum of the C1D1 and the C1D2 of obinutuzumab is about 38 mg/kg (e.g., 38 mg/kg±0.25 mg/kg, ±0.5 mg/kg, ±1 mg/kg, ±2 mg/kg, ±3 mg/kg, or ±3.8 mg/kg), about 28 mg/kg (e.g., 28 mg/kg±0.25 mg/kg, ±0.5 mg/kg, ±1 mg/kg, ±2 mg/kg, or ±2.8 mg/kg), about 23 mg/kg (e.g., 23 mg/kg±0.25 mg/kg, ±0.5 mg/kg, ±1 mg/kg, ±2 mg/kg, or ±2.3 mg/kg), about 20 mg/kg (e.g., 23 mg/kg±0.25 mg/kg, ±0.5 mg/kg, ±1 mg/kg, or ±2 mg/kg), or about 1000 mg (e.g., 1000 mg±5 mg, ±10 mg, ±20 mg, ±30 mg, ±50 mg, ±75 mg, or ±100 mg);
   (iii) administering a first dose (C1D1) of ifosfamide on Day 3, a second dose (C1D2) of ifosfamide on Day 4, and a third dose (C1D3) of ifosfamide on Day 5, wherein the C1D1, the C1D2, and the C1D3 of ifosfamide are each about 3000 mg/m² (e.g., 3000 mg/m²±40 mg/m², ±50 mg/m², ±100 mg/m², ±200 mg/m², or ±300 mg/m²);
   (iv) administering a single dose (C1D1) of carboplatin on Day 3, wherein the C1D1 of carboplatin is about 635 mg/m² (e.g., 635 mg/m²±5 mg/m², ±10 mg/m², ±25 mg/m², ±50 mg/m², ±60 mg/m², or ±63.5 mg/m²); and
   (v) administering a first dose (C1D1) of etoposide on Day 3, a second dose (C1D2) of etoposide on Day 4, and a third dose (C1D3) of etoposide on Day 5, wherein the C1D1, the C1D2, and the C1D3 of etoposide are each about 100 mg/m² (e.g., 100 mg/m²±1 mg/m², ±2.5 mg/m², ±5 mg/m², or ±10 mg/m²);
(b) the second dosing cycle comprises:
   (i) administering a single dose (C2D1) of glofitamab on Day 1, wherein the C2D1 of glofitamab is about 0.4 mg/kg (e.g., 0.4 mg/kg±0.005 mg/kg, ±0.01 mg/kg, ±0.02 mg/kg, ±0.03 mg/kg, or ±0.04 mg/kg), about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg), or about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg);
   (ii) administering a first dose (C2D1) of rituximab on Day 5, wherein the C2D1 of rituximab is about 375 mg/m² (e.g., 375 mg/m²±5 mg/m², ±10 mg/m², ±25 mg/m², or ±37.5 mg/m²);
   (iii) administering a first dose (C2D1) of ifosfamide on Day 6, a second dose (C2D2) of ifosfamide on Day 7, and a third dose (C2D3) of ifosfamide on Day 8, wherein the C2D1, the C2D2, and the C2D3 of ifosfamide are each about 3000 mg/m² (e.g., 3000 mg/m²±40 mg/m², ±50 mg/m², ±100 mg/m², ±200 mg/m², or ±300 mg/m²);
   (iv) administering a single dose (C2D1) of carboplatin on Day 6, wherein the C2D1 of carboplatin is about 5×(25+creatinine clearance) mg; and
   (v) administering a first dose (C2D1) of etoposide on Day 6, a second dose (C2D2) of etoposide on Day 7, and a third dose (C2D3) of etoposide on Day 8, wherein the C2D1, the C2D2, and the C2D3 of etoposide are each about 100 mg/m² (e.g., 100 mg/m²±1 mg/m², ±2.5 mg/m², ±5 mg/m², or ±10 mg/m²); and
(c) the third dosing cycle comprises:
   (i) administering a single dose (C3D1) of glofitamab on Day 1, wherein the C3D1 of glofitamab is about 0.4 mg/kg (e.g., 0.4 mg/kg±0.005 mg/kg, ±0.01 mg/kg, ±0.02 mg/kg, ±0.03 mg/kg, or ±0.04 mg/kg), about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg), or about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg);
   (ii) administering a first dose (C3D1) of rituximab on Day 5, wherein the C3D1 of rituximab is about 375 mg/m² (e.g., 375 mg/m²±5 mg/m², ±10 mg/m², ±25 mg/m², or ±37.5 mg/m²);
   (iii) administering a first dose (C3D1) of ifosfamide on Day 6, a second dose (C3D2) of ifosfamide on Day 7, and a third dose (C3D3) of ifosfamide on Day 8, wherein the C3D1, the C3D2, and the C3D3 of ifosfamide are each about 3000 mg/m² (e.g., 3000 mg/m²±40 mg/m², ±50 mg/m², ±100 mg/m², ±200 mg/m², or ±300 mg/m²);
   (iv) administering a single dose (C3D1) of carboplatin on Day 6, wherein the C3D1 of carboplatin is about 5×(25+creatinine clearance) mg; and
   (v) administering a first dose (C3D1) of etoposide on Day 6, a second dose (C3D2) of etoposide on Day 7, and a third dose (C3D3) of etoposide on Day 8, wherein the C3D1, the C3D2, and the C3D3 of etoposide are each about 100 mg/m² (e.g., 100 mg/m²±1 mg/m², ±2.5 mg/m², ±5 mg/m², or ±10 mg/m²).

In one embodiment, mesna is administered to the subject on Days 3, 4, and 5 of the first dosing cycle, on Days 6, 7, and 8 of the second dosing cycle, and/or on Days 6, 7, and 8 of each additional dosing cycle. In one embodiment, mesna is administered intravenously daily as five doses totaling 3000 mg/m² in amount. In one embodiment, mesna is administered intravenously at a first dose of about 600 mg/m² prior to the administration of any dose of ifosfamide and at four repeated doses of about 600 mg/m² each at about three hours, about six hours, about nine hours, and about 12 hours, respectively, after the first dose of ifosfamide.

In one aspect, the invention features a method of treating a subject aged between 18 years and 30 years having a CD20-positive cell proliferative disorder comprising administering to the subject an effective amount of glofitamab, obinutuzumab, rituximab, ifosfamide, carboplatin, and etoposide in a dosing regimen comprising at least a first dosing cycle and a second dosing cycle, wherein
(a) the first dosing cycle comprises administering a first dose (C1D1) of glofitamab on Day 8 and administering a second dose (C1D2) of glofitamab on Day 15, wherein the C1D1 of glofitamab is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 10 mg (e.g., 10 mg±0.05 mg, ±0.1 mg, ±0.2 mg, ±0.3 mg, ±0.5 mg, ±0.75 mg, or ±1 mg); and
(b) the second dosing cycle comprises administering a single dose (C2D1) of glofitamab on Day 1, wherein the C2D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one aspect, the invention features a method of treating a subject aged between 18 years and 30 years having a CD20-positive cell proliferative disorder comprising administering to the subject an effective amount of glofitamab, obinutuzumab, rituximab, ifosfamide, carboplatin, and etoposide in a dosing regimen comprising a first dosing cycle, a second dosing cycle, and a third dosing cycle, wherein
(a) the first dosing cycle comprises administering a first dose (C1D1) of glofitamab on Day 8 and administering a second dose (C1D2) of glofitamab on Day 15, wherein the C1D1 of glofitamab is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 10 mg (e.g., 10 mg±0.05 mg, ±0.1 mg, ±0.2 mg, ±0.3 mg, ±0.5 mg, ±0.75 mg, or ±1 mg);
(b) the second dosing cycle comprises administering a single dose (C2D1) of glofitamab on Day 1, wherein the C2D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or 3 mg); and
(c) the third dosing cycle comprises administering a single dose (C3D1) of glofitamab on Day 1, wherein the C3D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one aspect, the invention features a method of treating a subject aged between 18 years and 30 years having a CD20-positive cell proliferative disorder comprising administering to the subject an effective amount of glofitamab, obinutuzumab, rituximab, ifosfamide, carboplatin, and etoposide in a dosing regimen comprising at least a first dosing cycle and a second dosing cycle, wherein
(a) the first dosing cycle comprises:
   (i) administering a first dose (C1D1) of glofitamab on Day 8 and a second dose (C1D2) of glofitamab on Day 15, wherein the C1D1 of glofitamab is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 10 mg (e.g., 10 mg±0.05 mg, ±0.1 mg, ±0.2 mg, ±0.3 mg, ±0.5 mg, ±0.75 mg, or ±1 mg);
   (ii) administering a first dose (C1D1) of obinutuzumab on Day 1 and a second dose (C1D2) of obinutuzumab on Day 2, wherein the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab, and wherein the sum of the C1D1 and the C1D2 of obinutuzumab is about 1000 mg (e.g., 1000 mg±5 mg, ±10 mg, ±20 mg, ±30 mg, ±50 mg, ±75 mg, or 100 mg);
   (iii) administering a single dose (C1D1) of ifosfamide on Day 3, wherein the C1D1 of ifosfamide is about 5000 mg/m² (e.g., 5000 mg/m²±50 mg/m², ±100 mg/m², ±200 mg/m², ±300 mg/m², ±400 mg/m², or ±500 mg/m²) with a maximum dose of about 800 mg (e.g., 800 mg±10 mg, ±25 mg, ±50 mg, or ±80 mg);
   (iv) administering a single dose (C1D1) of carboplatin on Day 3, wherein the C1D1 of carboplatin is about 5×(25+creatinine clearance) mg; and
   (v) administering a first dose (C1D1) of etoposide on Day 3, a second dose (C1D2) of etoposide on Day 4, and a third dose (C1D3) of etoposide on Day 5, wherein the C1D1, the C1D2, and the C1D3 of etoposide are each about 100 mg/m² (e.g., 100 mg/m²±1 mg/m², ±2.5 mg/m², ±5 mg/m², or ±10 mg/m²); and
(b) the second dosing cycle comprises:
   (i) administering a single dose (C2D1) of glofitamab on Day 1, wherein the C2D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg);
   (ii) administering a first dose (C2D1) of rituximab on Day 5, wherein the C2D1 of rituximab is about 375 mg/m² (e.g., 375 mg/m²±5 mg/m², ±10 mg/m², ±25 mg/m², or ±37.5 mg/m²);
   (iii) administering a single dose (C2D1) of ifosfamide on Day 6, wherein the C2D1 of ifosfamide is about 5000 mg/m² (e.g., 5000 mg/m²±50 mg/m², ±100 mg/m², ±200 mg/m², ±300 mg/m², ±400 mg/m², or ±500 mg/m²) with a maximum dose of about 800 mg (e.g., 800 mg±10 mg, ±25 mg, ±50 mg, or ±80 mg);
   (iv) administering a single dose (C2D1) of carboplatin on Day 6, wherein the C2D1 of carboplatin is about 5×(25+creatinine clearance) mg; and
   (v) administering a first dose (C2D1) of etoposide on Day 6, a second dose (C2D2) of etoposide on Day 7, and a third dose (C2D3) of etoposide on Day 8, wherein the C2D1, the C2D2, and the C2D3 of etoposide are each about 100 mg/m² (e.g., 100 mg/m²±1 mg/m², ±2.5 mg/m², ±5 mg/m², or ±10 mg/m²).

In one aspect, the invention features a method of treating a subject aged between 18 years and 30 years having a CD20-positive cell proliferative disorder comprising administering to the subject an effective amount of glofitamab, obinutuzumab, rituximab, ifosfamide, carboplatin, and etoposide in a dosing regimen comprising a first dosing cycle, a second dosing cycle, and a third dosing cycle, wherein
(a) the first dosing cycle comprises:
   (i) administering a first dose (C1D1) of glofitamab on Day 8 and a second dose (C1D2) of glofitamab on Day 15, wherein the C1D1 of glofitamab is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 10 mg (e.g., 10 mg±0.05 mg, ±0.1 mg, ±0.2 mg, ±0.3 mg, ±0.5 mg, ±0.75 mg, or ±1 mg);
   (ii) administering a first dose (C1D1) of obinutuzumab on Day 1 and a second dose (C1D2) of obinutuzumab on Day 2, wherein the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab, and wherein the sum of the C1D1 and the C1D2 of obinutuzumab is about 1000 mg (e.g., 1000 mg±5 mg, ±10 mg, ±20 mg, ±30 mg, ±50 mg, ±75 mg, or 100 mg);
   (iii) administering a single dose (C1D1) of ifosfamide on Day 3, wherein the C1D1 of ifosfamide is about 5000 mg/m² (e.g., 5000 mg/m²±50 mg/m², ±100 mg/m², ±200 mg/m², ±300 mg/m², ±400 mg/m², or ±500 mg/m²) with a maximum dose of about 800 mg (e.g., 800 mg±10 mg, ±25 mg, ±50 mg, or ±80 mg);
   (iv) administering a single dose (C1D1) of carboplatin on Day 3, wherein the C1D1 of carboplatin is about 5×(25+creatinine clearance) mg; and
   (v) administering a first dose (C1D1) of etoposide on Day 3, a second dose (C1D2) of etoposide on Day 4, and a third dose (C1D3) of etoposide on Day 5, wherein the C1D1, the C1D2, and the C1D3 of etoposide are each about 100 mg/m² (e.g., 100 mg/m²±1 mg/m², ±2.5 mg/m², ±5 mg/m², or ±10 mg/m²);
(b) the second dosing cycle comprises:
   (i) administering a single dose (C2D1) of glofitamab on Day 1, wherein the C2D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg);
   (ii) administering a first dose (C2D1) of rituximab on Day 5, wherein the C2D1 of rituximab is about 375 mg/m² (e.g., 375 mg/m²±5 mg/m², ±10 mg/m², ±25 mg/m², or ±37.5 mg/m²);
   (iii) administering a single dose (C2D1) of ifosfamide on Day 6, wherein the C2D1 of ifosfamide is about 5000 mg/m² (e.g., 5000 mg/m²±50 mg/m², ±100 mg/m², ±200 mg/m², ±300 mg/m², ±400 mg/m², or ±500 mg/m²) with a maximum dose of about 800 mg (e.g., 800 mg±10 mg, ±25 mg, ±50 mg, or ±80 mg);
   (iv) administering a single dose (C2D1) of carboplatin on Day 6, wherein the C2D1 of carboplatin is about 5×(25+creatinine clearance) mg; and
   (v) administering a first dose (C2D1) of etoposide on Day 6, a second dose (C2D2) of etoposide on Day 7, and a third dose (C2D3) of etoposide on Day 8, wherein the C2D1, the C2D2, and the C2D3 of etoposide are each about 100 mg/m² (e.g., 100 mg/m²±1 mg/m², ±2.5 mg/m², ±5 mg/m², or ±10 mg/m²); and
(c) the third dosing cycle comprises:
   (i) administering a single dose (C3D1) of glofitamab on Day 1, wherein the C3D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg);
   (ii) administering a first dose (C3D1) of rituximab on Day 5, wherein the C3D1 of rituximab is about 375 mg/m² (e.g., 375 mg/m²±5 mg/m², ±10 mg/m², ±25 mg/m², or ±37.5 mg/m²);
   (iii) administering a single dose (C3D1) of ifosfamide on Day 6, wherein the C3D1 of ifosfamide is about 5000 mg/m² (e.g., 5000 mg/m²±50 mg/m², ±100 mg/m², ±200 mg/m², ±300 mg/m², ±400 mg/m², or ±500 mg/m²) with a maximum dose of about 800 mg (e.g., 800 mg±10 mg, ±25 mg, ±50 mg, or ±80 mg);
   (iv) administering a single dose (C3D1) of carboplatin on Day 6, wherein the C3D1 of carboplatin is about 5×(25+creatinine clearance) mg; and
   (v) administering a first dose (C3D1) of etoposide on Day 6, a second dose (C3D2) of etoposide on Day 7, and a third dose (C3D3) of etoposide on Day 8, wherein the C3D1, the C3D2, and the C3D3 of etoposide are each about 100 mg/m² (e.g., 100 mg/m²±1 mg/m², ±2.5 mg/m², ±5 mg/m², or ±10 mg/m²).

In one embodiment, mesna is administered simultaneously with any dose of ifosfamide. In one embodiment, mesna is administered intravenously at a dose of about 5000 mg/m² (e.g., 5000 mg/m²±50 mg/m², ±100 mg/m², ±200 mg/m², ±300 mg/m², ±400 mg/m², or ±500 mg/m²). In one embodiment, mesna is administered via continuous infusion over about 24 hours on Day 3 of the first dosing cycle, on Day 6 of the second dosing cycle, and/or on Day 6 of each additional dosing cycle. In one embodiment, the CD20-positive cell proliferative disorder is a relapsed and/or refractory DLBCL. In one embodiment, the CD20-positive cell proliferative disorder is a relapsed and/or refractory mature B cell NHL.

In one embodiment, the bispecific antibody that binds to CD20 and CD3 comprises at least one Fab molecule which specifically binds to CD20 comprising the following six hypervariable regions (HVRs):
(i) an HVR-H1 comprising the amino acid sequence of YSWIN (SEQ ID NO: 1);
(ii) an HVR-H2 comprising the amino acid sequence of RIFPGDGDTDYNGKFKG (SEQ ID NO: 2);
(iii) an HVR-H3 comprising the amino acid sequence of NVFDGYWLVY (SEQ ID NO:3);
(iv) an HVR-L1 comprising the amino acid sequence of RSSKSLLHSNGITYLY (SEQ ID NO: 4);
(v) an HVR-L2 comprising the amino acid sequence of QMSNLVS (SEQ ID NO: 5); and
(vi) an HVR-L3 comprising the amino acid sequence of AQNLELPYT (SEQ ID NO: 6).

In one embodiment, the bispecific antibody that binds to CD20 and CD3 comprises at least one Fab molecule which specifically binds to CD20 comprising (a) a heavy chain variable (VH) domain comprising an amino acid sequence having at least 95% sequence identity to the amino acid sequence of SEQ ID NO: 7; (b) a light chain variable (VL) domain comprising an amino acid sequence having at least 95% sequence identity to the amino acid sequence of SEQ ID NO: 8; or (c) a VH domain as in (a) and a VL domain as in (b).

In one embodiment, the Fab molecule which specifically binds to CD20 comprises (a) a VH domain comprising an amino acid sequence of SEQ ID NO: 7 and (b) a VL domain comprising an amino acid sequence of SEQ ID NO: 8.

In one embodiment, the bispecific antibody that binds to CD20 and CD3 comprises at least one Fab molecule which specifically binds to CD3 comprising the following six HVRs:
(i) an HVR-H1 comprising the amino acid sequence of TYAMN (SEQ ID NO: 9);
(ii) an HVR-H2 comprising the amino acid sequence of RIRSKYNNYATYYADSVKG (SEQ ID NO: 10);
(iii) an HVR-H3 comprising the amino acid sequence of HGNFGNSYVSWFAY (SEQ ID NO: 11);
(iv) an HVR-L1 comprising the amino acid sequence of GSSTGAVTTSNYAN (SEQ ID NO: 12);
(v) an HVR-L2 comprising the amino acid sequence of GTNKRAP (SEQ ID NO: 13); and
(vi) an HVR-L3 comprising the amino acid sequence of ALWYSNLWV (SEQ ID NO: 14).

In one embodiment, the bispecific antibody that binds to CD20 and CD3 comprises at least one Fab molecule which specifically binds to CD3 comprising (a) a heavy chain variable (VH) domain comprising an amino acid sequence having at least 95% sequence identity to the amino acid sequence of SEQ ID NO: 15; (b) a light chain variable (VL) domain comprising an amino acid sequence having at least 95% sequence identity to the amino acid sequence of SEQ ID NO: 16; or (c) a VH domain as in (a) and a VL domain as in (b).

In one embodiment, the Fab molecule which specifically binds to CD3 comprises (a) a VH domain comprising an amino acid sequence of SEQ ID NO: 15 and (b) a VL domain comprising an amino acid sequence of SEQ ID NO: 16.

In one embodiment, the bispecific antibody that binds to CD20 and CD3 is bivalent for CD20 and monovalent for CD3. In one embodiment, the bispecific antibody that binds to CD20 and CD3 comprises two Fab molecules which specifically bind to CD20 and one Fab molecule which specifically binds to CD3. In one embodiment, the bispecific antibody that binds to CD20 and CD3 is a humanized antibody. In one embodiment, the bispecific antibody that binds to CD20 and CD3 is glofitamab.

In one embodiment, the bispecific antibody that binds to CD20 and CD3 is administered intravenously.

In one embodiment, the anti-CD20 antibody is administered intravenously.

In one embodiment, the CD20-positive cell proliferative disorder is a B cell proliferative disorder. In one embodiment, the B cell proliferative disorder is a non-Hodgkin's lymphoma (NHL) or a central nervous system lymphoma (CNSL). In one embodiment, the NHL is a diffuse-large B cell lymphoma (DLBCL), a follicular lymphoma (FL), a mantle cell lymphoma (MCL), a marginal zone lymphoma (MZL), a high-grade B cell lymphoma, a primary mediastinal (thymic) large B cell lymphoma (PMLBCL), a diffuse B cell lymphoma, or a small lymphocytic lymphoma. In one embodiment, the NHL is a Burkitt lymphoma (BL) or a Burkitt leukemia (BAL). In one embodiment, the NHL is aggressive and/or mature. In one embodiment, the NHL is relapsed and/or refractory. In one embodiment, the B cell proliferative disorder is a relapsed and/or refractory mature B cell NHL. In one embodiment, the subject has received one prior systemic therapy. In one embodiment, the subject has received no more than one prior systemic therapy. In one embodiment, the prior systemic therapy comprises an anti-CD20 antibody and an anthracycline.

In one embodiment, the subject is human. In one embodiment, the subject is transplant or CAR-T cell therapy eligible.

In one embodiment, the subject receives autologous stem cell transplantation (ASCT) after completion of the dosing regimen as described above. In one embodiment, the ASCT is an autologous hematopoietic stem cell transplant. In one embodiment, the subject receives allogenic hematopoietic stem cell transplant after completion of the dosing regimen as described above. In one embodiment, the subject receives CAR-T cell therapy after completion of the dosing regimen as described above.

In one aspect, the invention features a bispecific antibody that binds to CD20 and CD3 for use in a method of treating a subject having a CD20-positive cell proliferative disorder, wherein said bispecific antibody that binds to CD20 and CD3 is administered in combination with an anti-CD20 antibody and one or more chemotherapeutic agents selected from ifosfamide, carboplatin and/or etoposide, in a dosing regimen comprising at least a first dosing cycle and a second dosing cycle.

In one embodiment, the first dosing cycle comprises a first dose (C1D1) of the bispecific antibody that binds to CD20 and CD3 and a second dose (C1D2) of the bispecific antibody, wherein the C1D1 of the bispecific antibody is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of the bispecific antibody is about 10 mg (e.g., 10 mg±0.05 mg, ±0.1 mg, ±0.2 mg, ±0.3 mg, ±0.5 mg, ±0.75 mg, or ±1 mg); and
the second dosing cycle comprises a single dose (C2D1) of the bispecific antibody, wherein the C2D1 of the bispecific antibody is about 10 mg (e.g., 10 mg±0.05 mg, ±0.1 mg, ±0.2 mg, ±0.3 mg, ±0.5 mg, ±0.75 mg, or ±1 mg), about 16 mg (e.g., 16 mg±0.05 mg, ±0.1 mg, ±0.2 mg, ±0.3 mg, ±0.5 mg, ±0.75 mg, ±1 mg, ±1.5 mg, or ±1.6 mg), or about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg). In one embodiment, the C2D1 of the bispecific antibody is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg). In one embodiment, the C2D1 of the bispecific antibody is administered to the subject on Day 8 of the second dosing cycle.

In one embodiment, the anti-CD20 antibody is obinutuzumab and/or rituximab. In one embodiment, the first dosing cycle comprises a single dose (C1D1) of obinutuzumab; and the second dosing cycle comprises a single dose (C2D1) of rituximab. In one embodiment, the single dose C1D1 of the obinutuzumab is about 1000 mg (e.g., 1000 mg±5 mg, ±10 mg, ±20 mg, ±30 mg, ±50 mg, ±75 mg, or ±100 mg) and the single dose of rituximab is about 375 mg/m² (e.g., 375 mg/m²±5 mg/m², ±10 mg/m², ±25 mg/m², or ±37.5 mg/m²).

In one embodiment, the anti-CD20 antibody is administered in a dosing regimen comprising at least a first dosing cycle and a second dosing cycle, wherein the first dosing cycle comprises a single dose (C1D1) of obinutuzumab on Day 1; and the second dosing cycle comprises a single dose (C2D1) of rituximab on Day 1.

In one aspect, the invention features a bispecific antibody that binds to CD20 and CD3 for use in a method of treating a subject having a CD20-positive cell proliferative disorder, wherein said bispecific antibody that binds to CD20 and CD3 is administered in combination with an anti-CD20 antibody, ifosfamide, carboplatin and etoposide, in a dosing regimen comprising at least a first dosing cycle and a second dosing cycle.

In one embodiment, the first dosing cycle comprises a single dose (C1D1) of ifosfamide, a single dose (C1D1) of carboplatin and a first (C1D1), second (C1D2) and third (C1D3) dose of etoposide; and the second cycle each comprises a single dose (C2D1) of ifosfamide, a single dose (C2D1) of carboplatin and a first (C2D1), second (C2D2) and third (C2D3) dose of etoposide. In one embodiment, ifosfamide is administered at a dose of about 5000 mg/m² (e.g., 5000 mg/m²±50 mg/m², ±100 mg/m², ±200 mg/m², ±300 mg/m², ±400 mg/m², or ±500 mg/m²), about 4000 mg/m² (e.g., 4000 mg/m²±40 mg/m², ±50 mg/m², ±100 mg/m², ±200 mg/m², ±300 mg/m², or ±400 mg/m²), or about 1666 mg/m² (e.g., 1666 mg/m²±25 mg/m², ±50 mg/m², ±100 mg/m², or ±166.6 mg/m²), carboplatin is administered at a dose in mg to target area under the curve (AUC) of about 5 mg/mL/min (e.g., 5 mg/mL/min±0.05 mg/mL/min, ±0.1 mg/mL/min, ±0.25 mg/mL/min, or ±0.5 mg/mL/min) with maximum dose of about 750 mg (e.g., 750 mg±10 mg, ±25 mg, ±50 mg, or ±75 mg) and etoposide is administered at a dose of about 100 mg/m² (e.g., 100 mg/m²±1 mg/m², ±2.5 mg/m², ±5 mg/m², or ±10 mg/m²) or 75 mg/m² (e.g., 0.5 mg/m²±1 mg/m², ±2.5 mg/m², ±5 mg/m², or ±7.5 mg/m²). In one embodiment, ifosfamide is administered at a dose of 5000 mg/m², 4000 mg/m² or 1666 mg/m², carboplatin is administered at a dose in mg to target area under the curve (AUC) of 5 mg/mL/min with maximum dose of 750 mg and etoposide is administered at a dose of 100 mg/m² or 75 mg/m². In one embodiment, ifosfamide is administered at a dose of 5000 mg/m², carboplatin is administered at a dose in mg to target area under the curve (AUC) of 5 mg/mL/min with maximum dose of 750 mg and etoposide is administered at a dose of 100 mg/m². In one embodiment, ifosfamide and carboplatin are administered on Day 2 of the first and second dosing cycle and etoposide is administered on each of Days 1, 2 and 3 of the first and second dosing cycle.

In one embodiment, the first and second dosing cycles are 21-day dosing cycles.

In one embodiment, the dosing regimen comprises one or more additional dosing cycles. In one embodiment, the additional dosing cycles are 21-day dosing cycles.

In one embodiment, the dosing regimen comprises three dosing cycles in total.

In one embodiment, the one or more additional dosing cycles comprise
(a) an additional single dose of the bispecific antibody that binds to CD20 and CD3,
(b) an additional single dose of the anti-CD20 antibody
(c) an additional single dose of ifosfamide, an additional single dose of carboplatin and an additional first, second and third dose of etoposide.

In one embodiment, the additional single dose of the bispecific antibody that binds to CD20 and CD3 is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one embodiment, the anti-CD20 antibody is rituximab. In one embodiment, the additional single dose of rituximab is about 375 mg/m² (e.g., 375 mg/m²±5 mg/m², ±10 mg/m², ±25 mg/m², or ±37.5 mg/m²). In one embodiment, the additional single dose of rituximab is administered on Day 1 of the additional dosing cycle.

In one embodiment, the additional single dose of ifosfamide is about 5000 mg/m² (e.g., 5000 mg/m²±50 mg/m², ±100 mg/m², ±200 mg/m², ±300 mg/m², ±400 mg/m², or ±500 mg/m²), about 4000 mg/m² (e.g., 4000 mg/m²±40 mg/m², ±50 mg/m², ±100 mg/m², ±200 mg/m², ±300 mg/m², or ±400 mg/m²), or about 1666 mg/m² (e.g., 1666 mg/m²±25 mg/m², ±50 mg/m², ±100 mg/m², or ±166.6 mg/m²), the additional single dose of carboplatin is in mg to target area under the curve (AUC) of about 5 mg/mL/min (e.g., 5 mg/mL/min±0.05 mg/mL/min, ±0.1 mg/mL/min, ±0.25 mg/mL/min, or ±0.5 mg/mL/min) with maximum dose of about 750 mg (e.g., 750 mg±10 mg, ±25 mg, ±50 mg, or ±75 mg) and the additional first, second and third dose of etoposide is 100 mg/m² (e.g., 100 mg/m²±1 mg/m², ±2.5 mg/m², ±5 mg/m², or ±10 mg/m²) or 75 mg/m² (e.g., 0.5 mg/m²±1 mg/m², ±2.5 mg/m², ±5 mg/m², or ±7.5 mg/m²). In one embodiment, the additional single dose of ifosfamide is 5000 mg/m², 4000 mg/m² or 1666 mg/m², the additional single dose of carboplatin is in mg to target area under the curve (AUC) of 5 mg/mL/min with maximum dose of 750 mg and the additional first, second and third dose of etoposide is 100 mg/m² or 75 mg/m².

In one embodiment, ifosfamide is administered at a dose of 5000 mg/m², carboplatin is administered at a dose in mg to target area under the curve (AUC) of 5 mg/mL/min with maximum dose of 750 mg and etoposide is administered at a dose of 100 mg/m².

In one embodiment, wherein ifosfamide and carboplatin are administered on Day 2 of the additional dosing cycle and etoposide is administered on each of Days 1, 2 and 3 of the additional dosing cycle.

In one embodiment, the method further comprises administering to the subject one or more additional therapeutic agents. In one embodiment, the bispecific antibody is for use in combination with one or more additional therapeutic agents. In one embodiment, the one or more additional therapeutic agent is tocilizumab. In one embodiment, the weight of the subject is greater than or equal to about 30 kg, and tocilizumab is administered at a dose of about 8 mg/kg (e.g., 8 mg/kg±0.05 mg/kg, ±0.1 mg/kg, ±0.25 mg/kg, ±0.5 mg/kg, or ±0.8 mg/kg). In one embodiment, the weight of the subject is less than 30 kg, and tocilizumab is administered at a dose of about 12 mg/kg (e.g., 12 mg/kg±0.05 mg/kg, ±0.1 mg/kg, ±0.25 mg/kg, ±0.5 mg/kg, ±0.75 mg/kg, ±1 mg/kg, or ±1.2 mg/kg). In some embodiments, the maximum dose of tocilizumab is about 800 mg (e.g., 800 mg±10 mg, ±25 mg, ±50 mg, or ±80 mg).

In one embodiment, the one or more additional therapeutic agents is a corticosteroid.

In one embodiment, the corticosteroid comprises prednisone, prednisolone, methylprednisolone, or dexamethasone. In one embodiment, dexamethasone is administered intravenously at a dose of about 20 mg (e.g., 20 mg±0.1 mg, ±0.25 mg, ±0.5 mg, ±1 mg, ±1.5 mg, or ±2 mg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the bispecific antibody. In one embodiment, dexamethasone is administered intravenously at a dose of about 20 mg (e.g., 20 mg±0.1 mg, ±0.25 mg, ±0.5 mg, ±1 mg, ±1.5 mg, or ±2 mg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of obinutuzumab. In one embodiment, wherein methylprednisolone is administered intravenously at a dose of about 80 mg (e.g., 80 mg±0.5 mg, ±1 mg, ±1.5 mg, ±2 mg, ±4 mg, ±6 mg, or ±8 mg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the bispecific antibody. In one embodiment, methylprednisolone is administered intravenously at a dose of about 80 mg (e.g., 80 mg±0.5 mg, ±1 mg, ±1.5 mg, ±2 mg, ±4 mg, ±6 mg, or ±8 mg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of obinutuzumab. In one embodiment, prednisone is administered orally at a dose of about 100 mg (e.g., 100 mg±0.5 mg, ±1 mg, ±1.5 mg, ±2 mg, ±4 mg, ±6 mg, ±8 mg, or ±10 mg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the bispecific antibody. In one embodiment, prednisolone is administered intravenously at a dose of about 100 mg (e.g., 100 mg±0.5 mg, ±1 mg, ±1.5 mg, ±2 mg, ±4 mg, ±6 mg, ±8 mg, or ±10 mg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the bispecific antibody.

In one embodiment, the one or more additional therapeutic agents is an antihistamine.

In one embodiment, the antihistamine is diphenhydramine. In one embodiment, diphenhydramine is administered orally or intravenously at a dose of about 50 mg (e.g., 50 mg±0.5 mg, ±1 mg, ±1.5 mg, ±2 mg, ±3 mg, ±4 mg, or ±5 mg) at least about 30 minutes (i.e., at least 30 minutes±3 minutes; e.g., at least about 1, 2, 3, 4, 5, 6, 7, 8, 9, 10, 11, 12, 18, 24, 36, 48 hours, or more) prior to the administration of any dose of the bispecific antibody.

In one embodiment, the one or more additional therapeutic agents comprises G-CSF. In one embodiment, G-CSF is administered between about one day and about two days (e.g., 24, 26, 28, 30, 32, 36, 38, 40, 42, 44, 46, or 48 hours) after administration of any dose of rituximab, ifosfamide, carboplatin, and/or etoposide.

In one embodiment, the one or more additional therapeutic agents is an antipyretic. In one embodiment, the antipyretic is acetaminophen or paracetamol. In one embodiment, acetaminophen or paracetamol is administered orally at a dose of between about 500 mg to about 1000 mg at least about 30 minutes (i.e., at least 30 minutes±3 minutes; e.g., at least about 1, 2, 3, 4, 5, 6, 7, 8, 9, 10, 11, 12, 18, 24, 36, 48 hours, or more) prior to the administration of any dose of the bispecific antibody. In one embodiment, acetaminophen or paracetamol is administered orally at a dose of between about 500 mg to about 1000 mg at least about 30 minutes (i.e., at least 30 minutes±3 minutes; e.g., at least about 1, 2, 3, 4, 5, 6, 7, 8, 9, 10, 11, 12, 18, 24, 36, 48 hours, or more) prior to the administration of any dose of obinutuzumab.

In one embodiment, the one or more additional therapeutic agents is mesna. In one embodiment, mesna is administered at a dose of about 5000 mg/m², about 4000 mg/m², or about 1666 mg/m² intravenously. In one embodiment, mesna is administered via continuous infusion over about 24 hours on Day 2 of each dosing cycle. In one embodiment, mesna is administered simultaneously with any dose of ifosfamide.

In one aspect, the invention features a bispecific antibody that binds to CD20 and CD3 for use in a method of treating a subject aged between 6 months and 17 years having a CD20-positive cell proliferative disorder, wherein the bispecific antibody that binds to CD20 and CD3 is to be administered in combination with an anti-CD20 antibody and one or more chemotherapeutic agents selected from ifosfamide, carboplatin, and/or etoposide.

in a dosing regimen comprising at least a first dosing cycle and a second dosing cycle.

In one embodiment, the first dosing cycle comprises a first dose (C1D1) of the bispecific antibody and a second dose (C1D2) of the bispecific antibody, wherein the C1D1 of the bispecific antibody is about 0.03 mg/kg (e.g., 0.03 mg/kg±0.0005 mg/kg, ±0.001 mg/kg, ±0.002 mg/kg, or ±0.003 mg/kg), about 0.04 mg/kg (e.g., 0.04 mg/kg±0.0005 mg/kg, ±0.001 mg/kg, ±0.002 mg/kg, ±0.003 mg/kg, or ±0.004 mg/kg), or about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of the bispecific antibody is about 0.15 mg/kg (e.g., 0.15 mg/kg±0.001 mg/kg, ±0.0025 mg/kg, ±0.005 mg/kg, ±0.01 mg/kg, or ±0.015 mg/kg) or about 10 mg (e.g., 10 mg±0.05 mg, ±0.1 mg, ±0.2 mg, ±0.3 mg, ±0.5 mg, ±0.75 mg, or ±1 mg); and
the second dosing cycle comprises a single dose (C2D1) of the bispecific antibody, wherein the C2D1 of the bispecific antibody is about 0.4 mg/kg (e.g., 0.4 mg/kg±0.005 mg/kg, ±0.01 mg/kg, ±0.02 mg/kg, ±0.03 mg/kg, or ±0.04 mg/kg), about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg), or about 30 mg (e.g., 30 mg±0.1 mg, ±0.2 mg, ±0.3 mg, ±0.5 mg, ±0.75 mg, ±1 mg, ±1.5 mg, ±2 mg, or 3 mg).

In one embodiment, (a) the subject's body weight is greater than or equal to about 7.5 kg and less than about 13 kg, and wherein the C1D1 of the bispecific antibody is about 0.04 mg/kg (e.g., 0.04 mg/kg±0.0005 mg/kg, ±0.001 mg/kg, ±0.002 mg/kg, ±0.003 mg/kg, or ±0.004 mg/kg), the C1D2 of the bispecific antibody is about 0.15 mg/kg (e.g., 0.15 mg/kg±0.001 mg/kg, ±0.0025 mg/kg, ±0.005 mg/kg, ±0.01 mg/kg, or ±0.015 mg/kg), and the C2D1 of the bispecific antibody is about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg);
(b) the subject's body weight is greater than or equal to about 13 kg and less than about 45 kg, and wherein the C1D1 of the bispecific antibody is about 0.03 mg/kg (e.g., 0.03 mg/kg±0.0005 mg/kg, ±0.001 mg/kg, ±0.002 mg/kg, or ±0.003 mg/kg), the C1D2 of the bispecific antibody is about 0.15 mg/kg (e.g., 0.15 mg/kg±0.001 mg/kg, ±0.0025 mg/kg, ±0.005 mg/kg, ±0.01 mg/kg, or ±0.015 mg/kg), and the C2D1 of the bispecific antibody is about 0.4 mg/kg (e.g., 0.4 mg/kg±0.005 mg/kg, ±0.01 mg/kg, ±0.02 mg/kg, ±0.03 mg/kg, or ±0.04 mg/kg); or
(c) the subject's body weight is greater than or equal to about 45 kg, and wherein the C1D1 of the bispecific antibody is about 2.5 mg (e.g., 2.5 mg±0.01 mg, ±0.02 mg, ±0.03 mg, ±0.05 mg, ±0.1 mg, ±0.02 mg, or ±0.25 mg), the C1D2 of the bispecific antibody is about 10 mg (e.g., 10 mg±0.05 mg, ±0.1 mg, ±0.2 mg, ±0.3 mg, ±0.5 mg, ±0.75 mg, or ±1 mg), and the C2D1 of the bispecific antibody is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one embodiment, the C1D1 of the bispecific antibody and the C1D2 of the bispecific antibody are to be administered to the subject on Days 8 and 15, respectively, of the first dosing cycle. In one embodiment, the C2D1 of the bispecific antibody is to be administered to the subject on Day 1 of the second dosing cycle.

In one embodiment, the anti-CD20 antibody is obinutuzumab and/or rituximab.

In one embodiment, the first dosing cycle comprises a first dose (C1D1) of obinutuzumab and a second dose (C1D2) of obinutuzumab. In one embodiment, (a) the subject's body weight is greater than or equal to about 7.5 kg and less than about 13 kg, and wherein the sum of the C1D1 and the C1D2 of obinutuzumab is about 38 mg/kg (e.g., 38 mg/kg±0.25 mg/kg, ±0.5 mg/kg, ±1 mg/kg, ±2 mg/kg, ±3 mg/kg, or ±3.8 mg/kg); (b) the subject's body weight is greater than or equal to about 13 kg and less than about 20 kg, and wherein the sum of the C1D1 and the C1D2 of obinutuzumab is about 28 mg/kg (e.g., 28 mg/kg±0.25 mg/kg, ±0.5 mg/kg, ±1 mg/kg, ±2 mg/kg, or ±2.8 mg/kg); (c) the subject's body weight is greater than or equal to about 20 kg and less than about 32 kg, and wherein the sum of the C1D1 and the C1D2 of obinutuzumab is about 23 mg/kg (e.g., 23 mg/kg±0.25 mg/kg, ±0.5 mg/kg, ±1 mg/kg, ±2 mg/kg, or ±2.3 mg/kg); (d) the subject's body weight is greater than or equal to about 32 kg and less than about 45 kg, and wherein the sum of the C1D1 and the C1D2 of obinutuzumab is about 20 mg/kg (e.g., 23 mg/kg±0.25 mg/kg, ±0.5 mg/kg, ±1 mg/kg, or ±2 mg/kg); or (e) the subject's body weight is greater than or equal to about 45 kg, and wherein the sum of the C1D1 and the C1D2 of obinutuzumab is about 1000 mg (e.g., 1000 mg±5 mg, ±10 mg, ±20 mg, ±30 mg, ±50 mg, ±75 mg, or ±100 mg).

In one embodiment, the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab. In one embodiment, (a) the subject's body weight is greater than or equal to about 7.5 kg and less than about 13 kg, and wherein the C1D1 of obinutuzumab is about 3.8 mg/kg (e.g., 3.8 mg/kg±0.05 mg/kg, 0.1 mg/kg, ±0.2 mg/kg, ±0.3 mg/kg, or ±0.38 mg/kg) and the C1D2 of obinutuzumab is about 34.2 mg/kg (e.g., 34.2 mg/kg±0.5 mg/kg, 1 mg/kg, ±2 mg/kg, ±3 mg/kg, or ±3.42 mg/kg); (b) the subject's body weight is greater than or equal to about 13 kg and less than about 20 kg, and wherein the C1D1 of obinutuzumab is about 2.8 mg/kg (e.g., 2.8 mg/kg±0.05 mg/kg, 0.1 mg/kg, ±0.2 mg/kg, or ±0.28 mg/kg) and the C1D2 of obinutuzumab is about 35.2 mg/kg (e.g., 35.2 mg/kg±0.5 mg/kg, 1 mg/kg, ±2 mg/kg, ±3 mg/kg, or ±3.52 mg/kg); (c) the subject's body weight is greater than or equal to about 20 kg and less than about 32 kg, and wherein the C1D1 of obinutuzumab is about 2.3 mg/kg (e.g., 2.3 mg/kg±0.05 mg/kg, 0.1 mg/kg, ±0.2 mg/kg, or ±0.23 mg/kg) and the C1D2 of obinutuzumab is about 35.7 mg/kg (e.g., 35.7 mg/kg±0.5 mg/kg, 1 mg/kg, ±2 mg/kg, ±3 mg/kg, or ±3.57 mg/kg); (d) the subject's body weight is greater than or equal to about 32 kg and less than about 45 kg, and wherein the C1D1 of obinutuzumab is about 2.0 mg/kg (e.g., 2.0 mg/kg±0.05 mg/kg, 0.1 mg/kg, or ±0.2 mg/kg) and the C1D2 of obinutuzumab is about 36.0 mg/kg (e.g., 36.0 mg/kg±0.5 mg/kg, 1 mg/kg, ±2 mg/kg, ±3 mg/kg, or ±3.6 mg/kg); or (e) the subject's body weight is greater than or equal to about 45 kg, and wherein the C1D1 of obinutuzumab is about 100 mg (e.g., 100 mg±0.5 mg, ±1 mg, ±1.5 mg, ±2 mg, ±4 mg, ±6 mg, ±8 mg, or ±10 mg) and the C1D2 of obinutuzumab is about 900 mg (e.g., 900 mg±5 mg, ±10 mg, ±20 mg, ±30 mg, ±40 mg, ±50 mg, ±60 mg, ±70 mg, ±80 mg, or ±90 mg).

In one embodiment, the C1D1 of obinutuzumab is to be administered to the subject on Day 1 of the first dosing cycle and the C1D2 of obinutuzumab is to be administered to the subject on Day 2 of the first dosing cycle.

In one embodiment, the second dosing cycle comprises a single dose (C2D1) of rituximab. In one embodiment, the C2D1 of rituximab is about 375 mg/m² (e.g., 375 mg/m²±5 mg/m², ±10 mg/m², ±25 mg/m², or ±37.5 mg/m²). In one embodiment, rituximab is to be administered to the subject on Day 5 of the second dosing cycle.

In one embodiment, the method comprises administering to the subject ifosfamide, carboplatin, and etoposide.

In one embodiment, the first dosing cycle comprises:
(a) a first dose (C1D1) of ifosfamide, a second dose (C1D2) of ifosfamide, and a third dose (C1D3) of ifosfamide;
(b) a single dose (C1D1) of carboplatin; and
(c) a first dose (C1D1) of etoposide, a second dose (C1D2) of etoposide, and a third dose (C1D3) of etoposide;
and the second cycle comprises:
(a) a first dose (C2D1) of ifosfamide, a second dose (C2D2) of ifosfamide, and a third dose (C2D3) of ifosfamide;
(b) a single dose (C2D1) of carboplatin; and
(c) a first dose (C2D1) of etoposide, a second dose (C2D2) of etoposide, and a third dose (C2D3) of etoposide.

In one embodiment, ifosfamide is to be administered at a dose of about 3000 mg/m² (e.g., 3000 mg/m²±40 mg/m², ±50 mg/m², ±100 mg/m², ±200 mg/m², or ±300 mg/m²) for each dose of ifosfamide, carboplatin is to be administered at a dose of about 635 mg/m² (e.g., 635 mg/m²±5 mg/m², ±10 mg/m², ±25 mg/m², ±50 mg/m², ±60 mg/m², or ±63.5 mg/m²), and etoposide is to be administered at a dose of about 100 mg/m² (e.g., 100 mg/m²±1 mg/m², ±2.5 mg/m², ±5 mg/m², or ±10 mg/m²) for each dose of etoposide. In one embodiment, (a) the C1D1, C1D2, and C1D3 of ifosfamide are to be administered on Days 3, 4, and 5, respectively of the first dosing cycle; (b) the C1D1 of carboplatin is to be administered on Day 3 of the first dosing cycle; (c) the C1D1, C1D2, and C1D3 of etoposide are to be administered on Days 3, 4, and 5, respectively, of the first dosing cycle; (d) the C2D1, C2D2, and C2D3 of ifosfamide are to be administered on Days 6, 7, and 8, respectively, of the second dosing cycle; (e) the C2D1 of carboplatin is to be administered on Day 6 of the second dosing cycle; and (f) the C2D1, C2D2, and C2D3 of etoposide are to be administered on Days 6, 7, and 8, respectively, of the second dosing cycle.

In one embodiment, the first and second dosing cycles are each 21-day dosing cycles. In one embodiment, the dosing regimen comprises one or more additional dosing cycles. In one embodiment, the one or more additional dosing cycles are each 21-day dosing cycles. In one embodiment, the dosing regimen comprises three dosing cycles in total.

In one embodiment, the one or more additional dosing cycles each comprises:
(a) an additional single dose of the bispecific antibody that binds to CD20 and CD3,
(b) an additional single dose of the anti-CD20 antibody, and
(c) an additional first dose, an additional second dose, and an additional third dose of ifosfamide; an additional single dose of carboplatin; and an additional first dose, an additional second dose, and an additional third dose of etoposide. In one embodiment, (a) the subject's body weight is greater than or equal to about 7.5 kg and less than about 13 kg, and wherein the additional single dose of the bispecific antibody is about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg); (b) the subject's body weight is greater than or equal to about 13 kg and less than about 45 kg, and wherein the additional single dose of the bispecific antibody is about 0.4 mg/kg (e.g., 0.4 mg/kg±0.005 mg/kg, ±0.01 mg/kg, ±0.02 mg/kg, ±0.03 mg/kg, or ±0.04 mg/kg); or (c) the subject's body weight is greater than or equal to about 45 kg, and wherein the additional single dose of the bispecific antibody is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one embodiment, the additional single dose of the bispecific antibody is to be administered to the subject on Day 1 of each of the one or more additional dosing cycles.

In one embodiment, the anti-CD20 antibody is rituximab. In one embodiment, the additional single dose of rituximab is about 375 mg/m² (e.g., 375 mg/m²±5 mg/m², ±10 mg/m², ±25 mg/m², or ±37.5 mg/m²). In one embodiment, the additional single dose of rituximab is to be administered on Day 5 of each of the one or more additional dosing cycles.

In one embodiment, the additional first dose, additional second dose, and additional third dose of ifosfamide are each about 3000 mg/m² (e.g., 3000 mg/m²±40 mg/m², ±50 mg/m², ±100 mg/m², ±200 mg/m², or ±300 mg/m²), the additional single dose of carboplatin is about 635 mg/m² (e.g., 635 mg/m²±5 mg/m², ±10 mg/m², ±25 mg/m², ±50 mg/m², ±60 mg/m², or ±63.5 mg/m²), and the additional first dose, the additional second dose, and the additional third dose of etoposide are each about 100 mg/m² (e.g., 100 mg/m²±1 mg/m², ±2.5 mg/m², ±5 mg/m², or ±10 mg/m²). In one embodiment, (a) the additional first dose, the additional second dose, and the additional third dose of ifosfamide are to be administered to the subject on Days 6, 7, and 8, respectively, of each of the one or more additional dosing cycles; (b) the additional single dose of carboplatin is to be administered on Day 6 of each of the one or more additional dosing cycles; and (c) the additional first dose, the additional second dose, and the additional third dose of etoposide are to be administered to the subject on Days 6, 7, and 8, respectively, of each of the one or more additional dosing cycles.

In one embodiment, the method further comprises administering to the subject one or more additional therapeutic agents.

In one embodiment, the one or more additional therapeutic agent is tocilizumab. In one embodiment, the weight of the subject is greater than or equal to about 30 kg and tocilizumab is to be administered at a dose of about 8 mg/kg (e.g., 8 mg/kg±0.05 mg/kg, ±0.1 mg/kg, ±0.25 mg/kg, ±0.5 mg/kg, or ±0.8 mg/kg) or the weight of the subject is less than 30 kg and tocilizumab is to be administered at a dose of about 12 mg/kg (e.g., 12 mg/kg±0.05 mg/kg, ±0.1 mg/kg, ±0.25 mg/kg, ±0.5 mg/kg, ±0.75 mg/kg, ±1 mg/kg, or ±1.2 mg/kg), and wherein the maximum dose is about 800 mg (e.g., 800 mg±10 mg, ±25 mg, ±50 mg, or ±80 mg).

In one embodiment, the one or more additional therapeutic agents is a corticosteroid. In one embodiment, the corticosteroid comprises prednisone, prednisolone, methylprednisolone, or dexamethasone.

In one embodiment, the corticosteroid is dexamethasone. In one embodiment, dexamethasone is to be administered intravenously at a dose of between about 0.15 mg/kg (e.g., 0.15 mg/kg±0.001 mg/kg, ±0.0025 mg/kg, ±0.005 mg/kg, ±0.01 mg/kg, or ±0.015 mg/kg) to about 0.5 mg/kg (e.g., 0.5 mg/kg±mg/kg, ±0.01 mg/kg, ±0.02 mg/kg, ±0.03 mg/kg, ±0.04 mg/kg, or ±0.05 mg/kg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the bispecific antibody, and wherein the maximum daily dose is 10 mg. In one embodiment, dexamethasone is to be administered intravenously at a dose of between about 0.15 mg/kg (e.g., 0.15 mg/kg±0.001 mg/kg, ±0.0025 mg/kg, ±0.005 mg/kg, ±0.01 mg/kg, or ±0.015 mg/kg) to about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of obinutuzumab, and wherein the maximum daily dose is 10 mg.

In one embodiment, the corticosteroid is methylprednisolone. In one embodiment, methylprednisolone is to be administered intravenously at a dose of between about 1 mg/kg to about 2 mg/kg (e.g., 1, 1.1, 1.2, 1.3, 1.4, 1.5, 1.6, 1.7, 1.9, or 2.0 mg/kg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the bispecific antibody. In one embodiment, methylprednisolone is to be administered intravenously at a dose of between about 1 mg/kg to about 2 mg/kg (e.g., 1, 1.1, 1.2, 1.3, 1.4, 1.5, 1.6, 1.7, 1.9, or 2.0 mg/kg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of obinutuzumab.

In one embodiment, the corticosteroid is prednisone or prednisolone. In one embodiment, prednisone or prednisolone is to be administered intravenously at a dose of about 100 mg (e.g., 100 mg±0.5 mg, ±1 mg, ±1.5 mg, ±2 mg, ±4 mg, ±6 mg, ±8 mg, or ±10 mg) or about 2 mg/kg at least about one hour (i.e., at least one hour±6 minutes; e.g., at least about 2, 3, 4, 5, 6, 7, 8, 9, 10, 11, 12, 18, 24, 36, 48 hours, or more) to the administration of any dose of the bispecific antibody. In one embodiment, prednisone or prednisolone is to be administered intravenously at a dose of about 100 mg (e.g., 100 mg±0.5 mg, ±1 mg, ±1.5 mg, ±2 mg, ±4 mg, ±6 mg, ±8 mg, or ±10 mg) or about 2 mg/kg at least about one hour ((i.e., at least one hour±6 minutes; e.g., at least about 2, 3, 4, 5, 6, 7, 8, 9, 10, 11, 12, 18, 24, 36, 48 hours, or more) prior to the administration of any dose of obinutuzumab.

In one embodiment, the one or more additional therapeutic agents is an antihistamine. In one embodiment, the antihistamine is diphenhydramine. In one embodiment, the subject is aged between two years and 17 years, and wherein diphenhydramine is to be administered intravenously at a dose of between about 10 mg to 20 mg (e.g., 10, 11, 12, 13, 14, 15, 16, 17, 18, 19, or 20 mg) with a maximum single dose of about 1.25 mg/kg. In one embodiment, the subject is aged less than two years, and wherein diphenhydramine is to be administered rectally at a dose of about 20 mg (e.g., 20 mg±0.1 mg, ±0.25 mg, ±0.5 mg, ±1 mg, ±1.5 mg, or ±2 mg). In one embodiment, diphenhydramine is to be administered at least about 30 minutes (i.e., at least 30 minutes±3 minutes; e.g., at least about 1, 2, 3, 4, 5, 6, 7, 8, 9, 10, 11, 12, 18, 24, 36, 48 hours, or more) prior to the administration of any dose of the bispecific antibody and/or the anti-CD20 antibody.

In one embodiment, the one or more additional therapeutic agents comprises granulocyte-colony stimulating factor (G-CSF). In one embodiment, G-CSF is to be administered between about one day and about two days (e.g., 24, 26, 28, 30, 32, 36, 38, 40, 42, 44, 46, or 48 hours) after administration of any dose of rituximab, ifosfamide, carboplatin, and/or etoposide. In one embodiment, G-CSF is to be administered intravenously or subcutaneously at a dose of about 5 μg/kg/day (e.g., 5 μg/kg/day±0.05 μg/kg/day, ±0.1 μg/kg/day, ±0.2 μg/kg/day, ±0.3 μg/kg/day, ±0.4 μg/kg/day, ±0.5 μg/kg/day), or about 10 μg/kg/day (e.g., 10 μg/kg/day±0.1 μg/kg/day, ±0.2 μg/kg/day, ±0.4 μg/kg/day, ±0.6 μg/kg/day, ±0.8 μg/kg/day, ±1 μg/kg/day). In one embodiment, G-CSF is to be administered at a dose of about 5 μg/kg/day (e.g., 5 μg/kg/day±0.05 μg/kg/day, ±0.1 μg/kg/day, ±0.2 μg/kg/day, ±0.3 μg/kg/day, ±0.4 μg/kg/day, ±0.5 μg/kg/day) in the first dosing cycle and about 10 μg/kg/day (e.g., 10 μg/kg/day±0.1 μg/kg/day, ±0.2 μg/kg/day, ±0.4 μg/kg/day, ±0.6 μg/kg/day, ±0.8 μg/kg/day, ±1 μg/kg/day) in the second dosing cycle and/or each additional dosing cycle.

In one embodiment, the one or more additional therapeutic agents is an antipyretic. In one embodiment, the antipyretic is acetaminophen or paracetamol. In one embodiment, acetaminophen or paracetamol is to be administered orally or intravenously at a dose of between about 500 to about 1000 mg (e.g., 500, 550, 600, 650, 700, 750, 800, 850, 900, 950, or 1000 mg). In one embodiment, acetaminophen or paracetamol is to be administered at least about 30 minutes (i.e., at least 30 minutes±3 minutes; e.g., at least about 1, 2, 3, 4, 5, 6, 7, 8, 9, 10, 11, 12, 18, 24, 36, 48 hours, or more) prior to the administration of any dose of the bispecific antibody and/or the anti-CD20 antibody.

In one embodiment, the one or more additional therapeutic agents is mesna. In one embodiment, mesna is to be administered intravenously daily as five doses totaling 3000 mg/m² in amount. In one embodiment, mesna is to be administered intravenously at a first dose of about 600 mg/m² prior to the administration of any dose of ifosfamide and at four repeated doses of about 600 mg/m² each at about three hours, about six hours, about nine hours, and about 12 hours, respectively, after the first dose of ifosfamide. In one embodiment, mesna is to be administered daily to the subject on Days 3, 4, and 5 of the first dosing cycle, on Days 6, 7, and 8 of the second dosing cycle, and/or on Days 6, 7, and 8 of each additional dosing cycle.

In one aspect, the invention features a bispecific antibody that binds to CD20 and CD3 for use in a method of treating a subject aged between 18 years and 30 years having a CD20-positive cell proliferative disorder, wherein the bispecific antibody that binds to CD20 and CD3 is to be administered in combination with an anti-CD20 antibody and one or more chemotherapeutic agents selected from ifosfamide, carboplatin, and/or etoposide
in a dosing regimen comprising at least a first dosing cycle and a second dosing cycle.

In one embodiment, the first dosing cycle comprises a first dose (C1D1) of the bispecific antibody and a second dose (C1D2) of the bispecific antibody, wherein the C1D1 of the bispecific antibody is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of the bispecific antibody is about 10 mg (e.g., 10 mg±0.05 mg, ±0.1 mg, ±0.2 mg, ±0.3 mg, ±0.5 mg, ±0.75 mg, or ±1 mg); and
the second dosing cycle comprises a single dose (C2D1) of the bispecific antibody, wherein the C2D1 of the bispecific antibody is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one embodiment, the C1D1 of the bispecific antibody and the C1D2 of the bispecific antibody are to be administered to the subject on Days 8 and 15, respectively, of the first dosing cycle.

In one embodiment, the C2D1 of the bispecific antibody is to be administered to the subject on Day 1 of the second dosing cycle.

In one embodiment, the anti-CD20 antibody is obinutuzumab and/or rituximab.

In one embodiment, the first dosing cycle comprises a first dose (C1D1) of obinutuzumab and a second dose (C1D2) of obinutuzumab. In one embodiment, the sum of the C1D1 and the C1D2 of obinutuzumab is about 1000 mg (e.g., 1000 mg±5 mg, ±10 mg, ±20 mg, ±30 mg, ±50 mg, ±75 mg, or ±100 mg). In one embodiment, the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab. In one embodiment, the C1D1 of obinutuzumab is about 100 mg (e.g., 100 mg±0.5 mg, ±1 mg, ±1.5 mg, ±2 mg, ±4 mg, ±6 mg, ±8 mg, or ±10 mg) and the C1D2 of obinutuzumab is about 900 mg (e.g., 900 mg±5 mg, ±10 mg, ±20 mg, ±30 mg, ±40 mg, ±50 mg, ±60 mg, ±70 mg, ±80 mg, or ±90 mg). In one embodiment, the C1D1 of obinutuzumab is to be administered to the subject on Day 1 of the first dosing cycle and the C1D2 of obinutuzumab is to be administered to the subject on Day 2 of the first dosing cycle.

In one embodiment, the second dosing cycle comprises a single dose (C2D1) of rituximab. In one embodiment, the C2D1 of rituximab is about 375 mg/m² (e.g., 375 mg/m²±5 mg/m², ±10 mg/m², ±25 mg/m², or ±37.5 mg/m²). In one embodiment, the C2D1 of rituximab is to be administered to the subject on Day 5 of the second dosing cycle.

In one embodiment, the method comprises administering to the subject ifosfamide, carboplatin, and etoposide. In one embodiment, the first dosing cycle comprises:
(a) a single dose (C1D1) of ifosfamide;
(b) a single dose (C1D1) of carboplatin; and
(c) a first dose (C1D1) of etoposide, a second dose (C1D2) of etoposide, and a third dose (C1D3) of etoposide;
and the second cycle comprises:
(a) a single dose (C2D1) of ifosfamide;
(b) a single dose (C2D1) of carboplatin; and
(c) a first dose (C2D1) of etoposide, a second dose (C2D2) of etoposide, and a third dose (C2D3) of etoposide.

In one embodiment, ifosfamide is to be administered at a dose of about 5000 mg/m² (e.g., 5000 mg/m²±50 mg/m², ±100 mg/m², ±200 mg/m², ±300 mg/m², ±400 mg/m², or ±500 mg/m²), carboplatin is to be administered at a dose of about 5×(25+CreatinineClearance (CrCl)) mg with maximum dose of about 750 mg (e.g., 750 mg±10 mg, ±25 mg, ±50 mg, or ±75 mg), and etoposide is to be administered at a dose of about 100 mg/m² (e.g., 100 mg/m²±1 mg/m², ±2.5 mg/m², ±5 mg/m², or ±10 mg/m²) for each dose of etoposide.

In one embodiment, (a) the subject is male, and CrCl is calculated using the formula CrCl=([140−age]×[weight in kg])/(72×[serum creatinine in mg/dL]); or (b) the subject is female, and CrCl is calculated using the formula CrCl=0.85×([140−age]×[weight in kg])/(72×[serum creatinine in mg/dL]).

In one embodiment, (a) the subject has CrCl<about 60 mL/min, and each single dose of ifosfamide is reduced to 4000 mg/m²; and/or (b) the subject has CrCl<about 50 mL/min, and each dose of etoposide is reduced to about 75 mg/m².

In one embodiment, (a) the C1D1 ifosfamide is to be administered on Day 3 of the first dosing cycle;
(b) the C1D1 of carboplatin is to be administered on Day 3 of the first dosing cycle;
(c) the C1D1, C1D2, and C1D3 of etoposide are to be administered on Days 3, 4, and 5, respectively, of the first dosing cycle;
(d) the C2D1 of ifosfamide is to be administered on Day 6 of the second dosing cycle;
(e) the C2D1 of carboplatin is to be administered on Day 6 of the second dosing cycle; and
(f) the C2D1, C2D2, and C2D3 of etoposide are to be administered on Days 6, 7, and 8, respectively, of the second dosing cycle.

In one embodiment, the first and second dosing cycles are each 21-day dosing cycles. In one embodiment, the dosing regimen comprises one or more additional dosing cycles. In one embodiment, the one or more additional dosing cycles are each 21-day dosing cycles. In one embodiment, the dosing regimen comprises three dosing cycles in total.

In one embodiment, the one or more additional dosing cycles each comprise:
(a) an additional single dose of the bispecific antibody that binds to CD20 and CD3,
(b) an additional single dose of the anti-CD20 antibody, and
(c) an additional single dose of ifosfamide; an additional single dose of carboplatin; and an additional first dose, an additional second dose, and an additional third dose of etoposide.

In one embodiment, the additional single dose of the bispecific antibody is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg). In one embodiment, the additional single dose of the bispecific antibody is to be administered to the subject on Day 1 of each of the one or more additional dosing cycles.

In one embodiment, the anti-CD20 antibody is rituximab. In one embodiment, the additional single dose of rituximab is about 375 mg/m² (e.g., 375 mg/m²±5 mg/m², ±10 mg/m², ±25 mg/m², or ±37.5 mg/m²). In one embodiment, the additional single dose of rituximab is to be administered on Day 5 of each of the one or more additional dosing cycles.

In one embodiment, the additional single dose of ifosfamide is about 5000 mg/m² (e.g., 5000 mg/m²±50 mg/m², ±100 mg/m², ±200 mg/m², ±300 mg/m², ±400 mg/m², or ±500 mg/m²), the additional single dose of carboplatin is about 5×(25+CreatinineClearance (CrCl)) mg with maximum dose of about 750 mg (e.g., 750 mg±10 mg, ±25 mg, ±50 mg, or ±75 mg), and the additional first dose, the additional second dose, and the additional third dose of etoposide are each about 100 mg/m² (e.g., 100 mg/m²±1 mg/m², ±2.5 mg/m², ±5 mg/m², or ±10 mg/m²).

In one embodiment, (a) the subject is male, and wherein CrCl is calculated using the formula CrCl=([140−age]×[weight in kg])/(72×[serum creatinine in mg/dL]); or (b) the subject is female, and wherein CrCl is calculated using the formula CrCl=0.85×([140−age]×[weight in kg])/(72×[serum creatinine in mg/dL]).

In one embodiment, (a) the subject has CrCl<about 60 mL/min, and the additional single dose of ifosfamide is reduced to 4000 mg/m²; and/or (b) the subject has CrCl<about 50 mL/min, and each additional dose of etoposide is reduced to about 75 mg/m².

In one embodiment, (a) the additional single dose of ifosfamide is to be administered on Day 6 of each of the one or more additional dosing cycles;
(b) the additional single dose of carboplatin is to be administered on Day 6 of each of the one or more additional dosing cycles; and
(c) the additional first dose, the additional second dose, and the additional third dose of etoposide are to be administered to the subject on Days 6, 7, and 8, respectively, of each of the one or more additional dosing cycles.

In one embodiment, the method further comprises administering to the subject one or more additional therapeutic agents.

In one embodiment, the one or more additional therapeutic agent is tocilizumab. In one embodiment, the weight of the subject is greater than or equal to about 30 kg and tocilizumab is to be administered at a dose of about 8 mg/kg (e.g., 8 mg/kg±0.05 mg/kg, ±0.1 mg/kg, ±0.25 mg/kg, ±0.5 mg/kg, or ±0.8 mg/kg) or the weight of the subject is less than 30 kg and tocilizumab is to be administered at a dose of about 12 mg/kg (e.g., 12 mg/kg±0.05 mg/kg, ±0.1 mg/kg, ±0.25 mg/kg, ±0.5 mg/kg, ±0.75 mg/kg, ±1 mg/kg, or ±1.2 mg/kg), and wherein the maximum dose is about 800 mg (e.g., 800 mg±10 mg, ±25 mg, ±50 mg, or ±80 mg).

In one embodiment, the one or more additional therapeutic agents is a corticosteroid.

In one embodiment, the corticosteroid comprises prednisone, prednisolone, methylprednisolone, or dexamethasone.

In one embodiment, the corticosteroid is dexamethasone. In one embodiment, dexamethasone is to be administered intravenously at a dose of between about 0.15 mg/kg (e.g., 0.15 mg/kg±0.001 mg/kg, ±0.0025 mg/kg, ±0.005 mg/kg, ±0.01 mg/kg, or ±0.015 mg/kg) to about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the bispecific antibody, and wherein the maximum daily dose is 10 mg. In one embodiment, dexamethasone is to be administered intravenously at a dose of between about 0.15 mg/kg (e.g., 0.15 mg/kg±0.001 mg/kg, ±0.0025 mg/kg, ±0.005 mg/kg, ±0.01 mg/kg, or ±0.015 mg/kg) to about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of obinutuzumab, and wherein the maximum daily dose is 10 mg.

In one embodiment, the corticosteroid is methylprednisolone. In one embodiment, methylprednisolone is to be administered intravenously at a dose of between about 1 mg/kg to about 2 mg/kg (e.g., 1, 1.1, 1.2, 1.3, 1.4, 1.5, 1.6, 1.7, 1.9, or 2.0 mg/kg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the bispecific antibody. In one embodiment, methylprednisolone is to be administered intravenously at a dose of between about 1 mg/kg to about 2 mg/kg (e.g., 1, 1.1, 1.2, 1.3, 1.4, 1.5, 1.6, 1.7, 1.9, or 2.0 mg/kg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of obinutuzumab.

In one embodiment, the corticosteroid is prednisone or prednisolone. In one embodiment, prednisone or prednisolone is to be administered intravenously at a dose of about 100 mg (e.g., 100 mg±0.5 mg, ±1 mg, ±1.5 mg, ±2 mg, ±4 mg, ±6 mg, ±8 mg, or ±10 mg) or about 2 mg/kg at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the bispecific antibody. In one embodiment, prednisone or prednisolone is to be administered intravenously at a dose of about 100 mg (e.g., 100 mg±0.5 mg, ±1 mg, ±1.5 mg, ±2 mg, ±4 mg, ±6 mg, ±8 mg, or ±10 mg) or about 2 mg/kg at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of obinutuzumab.

In one embodiment, the one or more additional therapeutic agents is an antihistamine. In one embodiment, the antihistamine is diphenhydramine. In one embodiment, diphenhydramine is to be administered orally or intravenously at a dose of about 50 mg (e.g., 50 mg±0.5 mg, ±1 mg, ±1.5 mg, ±2 mg, ±3 mg, ±4 mg, or ±5 mg). In one embodiment, diphenhydramine is to be administered at least about 30 minutes (i.e., at least 30 minutes±3 minutes; e.g., at least about 1, 2, 3, 4, 5, 6, 7, 8, 9, 10, 11, 12, 18, 24, 36, 48 hours, or more) prior to the administration of any dose of the bispecific antibody and/or the anti-CD20 antibody.

In one embodiment, the one or more additional therapeutic agents comprises granulocyte-colony stimulating factor (G-CSF). In one embodiment, G-CSF is to be administered between about one day and about two days (e.g., 24, 26, 28, 30, 32, 36, 38, 40, 42, 44, 46, or 48 hours) after administration of any dose of rituximab, ifosfamide, carboplatin, and/or etoposide. In one embodiment, G-CSF is to be administered intravenously or subcutaneously at a dose of about 5 μg/kg/day (e.g., 5 μg/kg/day±0.05 μg/kg/day, ±0.1 μg/kg/day, ±0.2 μg/kg/day, ±0.3 μg/kg/day, ±0.4 μg/kg/day, ±0.5 μg/kg/day) or about 10 μg/kg/day (e.g., 10 μg/kg/day±0.1 μg/kg/day, ±0.2 μg/kg/day, ±0.4 μg/kg/day, ±0.6 μg/kg/day, ±0.8 μg/kg/day, ±1 μg/kg/day). In one embodiment, G-CSF is to be administered at a dose of about 5 μg/kg/day (e.g., 5 μg/kg/day±0.05 μg/kg/day, ±0.1 μg/kg/day, ±0.2 μg/kg/day, ±0.3 μg/kg/day, ±0.4 μg/kg/day, ±0.5 μg/kg/day) in the first dosing cycle and about 10 μg/kg/day (e.g., 10 μg/kg/day±0.1 μg/kg/day, ±0.2 μg/kg/day, ±0.4 μg/kg/day, ±0.6 μg/kg/day, ±0.8 μg/kg/day, ±1 μg/kg/day) in the second dosing cycle and/or each additional dosing cycle.

In one embodiment, the one or more additional therapeutic agents is an antipyretic. In one embodiment, the antipyretic is acetaminophen or paracetamol. In one embodiment, acetaminophen or paracetamol is to be administered orally or intravenously at a dose of between about 500 to about 1000 mg (e.g., 500, 550, 600, 650, 700, 750, 800, 850, 900, 950, or 1000 mg). In one embodiment, acetaminophen or paracetamol is to be administered at least about 30 minutes (i.e., at least 30 minutes±3 minutes; e.g., at least about 1, 2, 3, 4, 5, 6, 7, 8, 9, 10, 11, 12, 18, 24, 36, 48 hours, or more) prior to the administration of any dose of the bispecific antibody and/or the anti-CD20 antibody.

In one embodiment, the one or more additional therapeutic agents is mesna. In one embodiment, mesna is to be administered intravenously at a dose of about 5000 mg/m² (e.g., 5000 mg/m²±50 mg/m², ±100 mg/m², ±200 mg/m², ±300 mg/m², ±400 mg/m², or ±500 mg/m²). In one embodiment, mesna is to be administered via continuous infusion over about 24 hours on Day 3 of the first dosing cycle, on Day 6 of the second dosing cycle, and/or on Day 6 of each additional dosing cycle. In one embodiment, mesna is to be administered simultaneously with any dose of ifosfamide.

In one aspect, the invention features glofitamab for use in a method of treating a subject having a CD20-positive cell proliferative disorder, wherein glofitamab is to be administered in combination with obinutuzumab, rituximab, ifosfamide, carboplatin, and etoposide in a dosing regimen comprising at least a first dosing cycle and a second dosing cycle, wherein
(a) the first dosing cycle comprises administering a first dose (C1D1) of glofitamab on Day 8 and administering a second dose (C1D2) of glofitamab on Day 15, wherein the C1D1 of glofitamab is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 10 mg (e.g., 10 mg±0.05 mg, ±0.1 mg, ±0.2 mg, ±0.3 mg, ±0.5 mg, ±0.75 mg, or ±1 mg); and
(b) the second dosing cycle comprises administering a single dose (C2D1) of glofitamab on Day 8, wherein the C2D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one aspect, the invention features glofitamab for use in a method of treating a subject having a CD20-positive cell proliferative disorder, wherein glofitamab is to be administered in combination with obinutuzumab, rituximab, ifosfamide, carboplatin, and etoposide in a dosing regimen comprising a first dosing cycle, a second dosing cycle, and a third dosing cycle, wherein
(a) the first dosing cycle comprises administering a first dose (C1D1) of glofitamab on Day 8 and administering a second dose (C1D2) of glofitamab on Day 15, wherein the C1D1 of glofitamab is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 10 mg (e.g., 10 mg±0.05 mg, ±0.1 mg, ±0.2 mg, ±0.3 mg, ±0.5 mg, ±0.75 mg, or ±1 mg);
(b) the second dosing cycle comprises administering a single dose (C2D1) of glofitamab on Day 8, wherein the C2D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg); and
(c) the third dosing cycle comprises administering a single dose (C3D1) of glofitamab on Day 8, wherein the C3D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one aspect, the invention features glofitamab for use in a method of treating a subject having a CD20-positive cell proliferative disorder, wherein glofitamab is to be administered in combination with obinutuzumab, rituximab, ifosfamide, carboplatin, and etoposide in a dosing regimen comprising at least a first dosing cycle and a second dosing cycle, wherein
(a) the first dosing cycle comprises:
   (i) administering a first dose (C1D1) of glofitamab on Day 8 and a second dose (C1D2) of glofitamab on Day 15, wherein the C1D1 of glofitamab is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 10 mg (e.g., 10 mg±0.05 mg, ±0.1 mg, ±0.2 mg, ±0.3 mg, ±0.5 mg, ±0.75 mg, or ±1 mg);
   (ii) administering a first dose (C1D1) of obinutuzumab on Day 1, wherein the C1D1 of obinutuzumab is about 1000 mg (e.g., 1000 mg±5 mg, ±10 mg, ±20 mg, ±30 mg, ±50 mg, ±75 mg, or ±100 mg);
   (iii) administering a single dose (C1D1) of ifosfamide on Day 2, wherein the C1D1 of ifosfamide is about 5000 mg/m² (e.g., 5000 mg/m²±50 mg/m², ±100 mg/m², ±200 mg/m², ±300 mg/m², ±400 mg/m², or ±500 mg/m²) with a maximum dose of about 800 mg (e.g., 800 mg±10 mg, ±25 mg, ±50 mg, or ±80 mg);
   (iv) administering a single dose (C1D1) of carboplatin on Day 2, wherein the C1D1 of carboplatin is about 5×(25+creatinine clearance) mg; and
   (v) administering a first dose (C1D1) of etoposide on Day 1, a second dose (C1D2) of etoposide on Day 2, and a third dose (C1D3) of etoposide on Day 3, wherein the C1D1, the C1D2, and the C1D3 of etoposide are each about 100 mg/m² (e.g., 100 mg/m²±1 mg/m², ±2.5 mg/m², ±5 mg/m², or ±10 mg/m²); and
(b) the second dosing cycle comprises:
   (i) administering a single dose (C2D1) of glofitamab on Day 8, wherein the C2D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg);
   (ii) administering a first dose (C2D1) of rituximab on Day 1, wherein the C2D1 of rituximab is about 375 mg/m² (e.g., 375 mg/m²±5 mg/m², ±10 mg/m², ±25 mg/m², or ±37.5 mg/m²);
   (iii) administering a single dose (C2D1) of ifosfamide on Day 2, wherein the C2D1 of ifosfamide is about 5000 mg/m² (e.g., 5000 mg/m²±50 mg/m², ±100 mg/m², ±200 mg/m², ±300 mg/m², ±400 mg/m², or ±500 mg/m²) with a maximum dose of about 800 mg (e.g., 800 mg±10 mg, ±25 mg, ±50 mg, or ±80 mg);
   (iv) administering a single dose (C2D1) of carboplatin on Day 2, wherein the C2D1 of carboplatin is about 5×(25+creatinine clearance) mg; and
   (v) administering a first dose (C2D1) of etoposide on Day 1, a second dose (C2D2) of etoposide on Day 2, and a third dose (C2D3) of etoposide on Day 3, wherein the C2D1, the C2D2, and the C2D3 of etoposide are each about 100 mg/m² (e.g., 100 mg/m²±1 mg/m², ±2.5 mg/m², ±5 mg/m², or ±10 mg/m²).

In one aspect, the invention features glofitamab for use in a method of treating a subject having a CD20-positive cell proliferative disorder, wherein glofitamab is to be administered in combination with obinutuzumab, rituximab, ifosfamide, carboplatin, and etoposide in a dosing regimen comprising a first dosing cycle, a second dosing cycle, and a third dosing cycle, wherein
(a) the first dosing cycle comprises:
   (i) administering a first dose (C1D1) of glofitamab on Day 8 and a second dose (C1D2) of glofitamab on Day 15, wherein the C1D1 of glofitamab is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 10 mg (e.g., 10 mg±0.05 mg, ±0.1 mg, ±0.2 mg, ±0.3 mg, ±0.5 mg, ±0.75 mg, or ±1 mg);
   (ii) administering a first dose (C1D1) of obinutuzumab on Day 1, wherein the C1D1 of obinutuzumab is about 1000 mg (e.g., 1000 mg±5 mg, ±10 mg, ±20 mg, ±30 mg, ±50 mg, ±75 mg, or ±100 mg);
   (iii) administering a single dose (C1D1) of ifosfamide on Day 2, wherein the C1D1 of ifosfamide is about 5000 mg/m² (e.g., 5000 mg/m²±50 mg/m², ±100 mg/m², ±200 mg/m², ±300 mg/m², ±400 mg/m², or ±500 mg/m²) with a maximum dose of about 800 mg (e.g., 800 mg±10 mg, ±25 mg, ±50 mg, or ±80 mg);
   (iv) administering a single dose (C1D1) of carboplatin on Day 2, wherein the C1D1 of carboplatin is about 5×(25+creatinine clearance) mg; and
   (v) administering a first dose (C1D1) of etoposide on Day 1, a second dose (C1D2) of etoposide on Day 2, and a third dose (C1D3) of etoposide on Day 3, wherein the C1D1, the C1D2, and the C1D3 of etoposide are each about 100 mg/m² (e.g., 100 mg/m²±1 mg/m², ±2.5 mg/m², ±5 mg/m², or ±10 mg/m²);
(b) the second dosing cycle comprises:
   (i) administering a single dose (C2D1) of glofitamab on Day 8, wherein the C2D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg);
   (ii) administering a first dose (C2D1) of rituximab on Day 1, wherein the C2D1 of rituximab is about 375 mg/m² (e.g., 375 mg/m²±5 mg/m², ±10 mg/m², ±25 mg/m², or ±37.5 mg/m²);
   (iii) administering a single dose (C2D1) of ifosfamide on Day 2, wherein the C2D1 of ifosfamide is about 5000 mg/m² (e.g., 5000 mg/m²±50 mg/m², ±100 mg/m², ±200 mg/m², ±300 mg/m², ±400 mg/m², or ±500 mg/m²) with a maximum dose of about 800 mg (e.g., 800 mg±10 mg, ±25 mg, ±50 mg, or ±80 mg);
   (iv) administering a single dose (C2D1) of carboplatin on Day 2, wherein the C2D1 of carboplatin is about 5×(25+creatinine clearance) mg; and
   (v) administering a first dose (C2D1) of etoposide on Day 1, a second dose (C2D2) of etoposide on Day 2, and a third dose (C2D3) of etoposide on Day 3, wherein the C2D1, the C2D2, and the C2D3 of etoposide are each about 100 mg/m² (e.g., 100 mg/m²±1 mg/m², ±2.5 mg/m², ±5 mg/m², or ±10 mg/m²); and
(c) the third dosing cycle comprises:
   (i) administering a single dose (C3D1) of glofitamab on Day 8, wherein the C3D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg);
   (ii) administering a first dose (C3D1) of rituximab on Day 1, wherein the C3D1 of rituximab is about 375 mg/m² (e.g., 375 mg/m²±5 mg/m², ±10 mg/m², ±25 mg/m², or ±37.5 mg/m²);
   (iii) administering a single dose (C3D1) of ifosfamide on Day 2, wherein the C3D1 of ifosfamide is about 5000 mg/m² (e.g., 5000 mg/m²±50 mg/m², ±100 mg/m², ±200 mg/m², ±300 mg/m², ±400 mg/m², or ±500 mg/m²) with a maximum dose of about 800 mg (e.g., 800 mg±10 mg, ±25 mg, ±50 mg, or ±80 mg);
   (iv) administering a single dose (C3D1) of carboplatin on Day 2, wherein the C3D1 of carboplatin is about 5×(25+creatinine clearance) mg; and
   (v) administering a first dose (C3D1) of etoposide on Day 1, a second dose (C3D2) of etoposide on Day 2, and a third dose (C3D3) of etoposide on Day 3, wherein the C3D1, the C3D2, and the C3D3 of etoposide are each about 100 mg/m² (e.g., 100 mg/m²±1 mg/m², ±2.5 mg/m², ±5 mg/m², or ±10 mg/m²).

In one embodiment, mesna is to be administered simultaneously with any dose of ifosfamide. In one embodiment, mesna is to be administered at a dose of about 5000 mg/m² (e.g., 5000 mg/m²±50 mg/m², ±100 mg/m², ±200 mg/m², ±300 mg/m², ±400 mg/m², or ±500 mg/m²) intravenously. In one embodiment, mesna is to be administered via continuous infusion over about 24 hours on Day 2 of each dosing cycle.

In one aspect, the invention features a method of treating a subject aged between 6 months and 17 years having a CD20-positive cell proliferative disorder comprising administering to the subject an effective amount of glofitamab, obinutuzumab, rituximab, ifosfamide, carboplatin, and etoposide in a dosing regimen comprising at least a first dosing cycle and a second dosing cycle, wherein
(a) the first dosing cycle comprises administering a first dose (C1D1) of glofitamab on Day 8 and administering a second dose (C1D2) of glofitamab on Day 15, wherein the C1D1 of glofitamab is about 0.03 mg/kg (e.g., 0.03 mg/kg±0.0005 mg/kg, ±0.001 mg/kg, ±0.002 mg/kg, or ±0.003 mg/kg), about 0.04 mg/kg (e.g., 0.04 mg/kg±0.0005 mg/kg, ±0.001 mg/kg, ±0.002 mg/kg, ±0.003 mg/kg, or ±0.004 mg/kg), or about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 0.15 mg/kg (e.g., 0.15 mg/kg±0.001 mg/kg, ±0.0025 mg/kg, ±0.005 mg/kg, ±0.01 mg/kg, or ±0.015 mg/kg) or about 10 mg (e.g., 10 mg±0.05 mg, ±0.1 mg, ±0.2 mg, ±0.3 mg, ±0.5 mg, ±0.75 mg, or ±1 mg); and
(b) the second dosing cycle comprises administering a single dose (C2D1) of glofitamab on Day 8, wherein the C2D1 of glofitamab is about 0.4 mg/kg (e.g., 0.4 mg/kg±0.005 mg/kg, ±0.01 mg/kg, ±0.02 mg/kg, ±0.03 mg/kg, or ±0.04 mg/kg), about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg), or about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one aspect, the invention features a method of treating a subject aged between 6 months and 17 years having a CD20-positive cell proliferative disorder comprising administering to the subject an effective amount of glofitamab, obinutuzumab, rituximab, ifosfamide, carboplatin, and etoposide in a dosing regimen comprising a first dosing cycle, a second dosing cycle, and a third dosing cycle, wherein
(a) the first dosing cycle comprises administering a first dose (C1D1) of glofitamab on Day 8 and administering a second dose (C1D2) of glofitamab on Day 15, wherein the C1D1 of glofitamab is about 0.03 mg/kg (e.g., 0.03 mg/kg±0.0005 mg/kg, ±0.001 mg/kg, ±0.002 mg/kg, or ±0.003 mg/kg), about 0.04 mg/kg (e.g., 0.04 mg/kg±0.0005 mg/kg, ±0.001 mg/kg, ±0.002 mg/kg, ±0.003 mg/kg, or ±0.004 mg/kg), or about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 0.15 mg/kg (e.g., 0.15 mg/kg±0.001 mg/kg, ±0.0025 mg/kg, ±0.005 mg/kg, ±0.01 mg/kg, or ±0.015 mg/kg) or about 10 mg (e.g., 10 mg±0.05 mg, ±0.1 mg, ±0.2 mg, ±0.3 mg, ±0.5 mg, ±0.75 mg, or ±1 mg);
(b) the second dosing cycle comprises administering a single dose (C2D1) of glofitamab on Day 8, wherein the C2D1 of glofitamab is about 0.4 mg/kg (e.g., 0.4 mg/kg±0.005 mg/kg, ±0.01 mg/kg, ±0.02 mg/kg, ±0.03 mg/kg, or ±0.04 mg/kg), about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg), or about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg); and
(c) the third dosing cycle comprises administering a single dose (C3D1) of glofitamab on Day 8, wherein the C3D1 of glofitamab is about 0.4 mg/kg (e.g., 0.4 mg/kg±0.005 mg/kg, ±0.01 mg/kg, ±0.02 mg/kg, ±0.03 mg/kg, or ±0.04 mg/kg), about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg), or about 30 mg (e.g., 30 mg±0.1 mg, ±0.2 mg, ±0.3 mg, ±0.5 mg, ±0.75 mg, ±1 mg, ±1.5 mg, ±2 mg, or 3 mg).

In one aspect, the invention features a method of treating a subject aged between 6 months and 17 years having a CD20-positive cell proliferative disorder comprising administering to the subject an effective amount of glofitamab, obinutuzumab, rituximab, ifosfamide, carboplatin, and etoposide in a dosing regimen comprising at least a first dosing cycle and a second dosing cycle, wherein
(a) the first dosing cycle comprises:
   (i) administering a first dose (C1D1) of glofitamab on Day 8 and a second dose (C1D2) of glofitamab on Day 15, wherein the C1D1 of glofitamab is about 0.03 mg/kg (e.g., 0.03 mg/kg±0.0005 mg/kg, ±0.001 mg/kg, ±0.002 mg/kg, or ±0.003 mg/kg), about 0.04 mg/kg (e.g., 0.04 mg/kg±0.0005 mg/kg, ±0.001 mg/kg, ±0.002 mg/kg, ±0.003 mg/kg, or ±0.004 mg/kg), or about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 0.15 mg/kg (e.g., 0.15 mg/kg±0.001 mg/kg, ±0.0025 mg/kg, ±0.005 mg/kg, ±0.01 mg/kg, or ±0.015 mg/kg) or about 10 mg (e.g., 10 mg±0.05 mg, ±0.1 mg, ±0.2 mg, ±0.3 mg, ±0.5 mg, ±0.75 mg, or ±1 mg);
   (ii) administering a first dose (C1D1) of obinutuzumab on Day 1 and a second dose (C1D2) of obinutuzumab on Day 2, wherein the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab, and wherein the sum of the C1D1 and the C1D2 of obinutuzumab is about 38 mg/kg (e.g., 38 mg/kg±0.25 mg/kg, ±0.5 mg/kg, ±1 mg/kg, ±2 mg/kg, ±3 mg/kg, or ±3.8 mg/kg), about 28 mg/kg (e.g., 28 mg/kg±0.25 mg/kg, ±0.5 mg/kg, ±1 mg/kg, ±2 mg/kg, or ±2.8 mg/kg), about 23 mg/kg (e.g., 23 mg/kg±0.25 mg/kg, ±0.5 mg/kg, ±1 mg/kg, ±2 mg/kg, or ±2.3 mg/kg), about 20 mg/kg, or about 1000 mg (e.g., 1000 mg±5 mg, ±10 mg, ±20 mg, ±30 mg, ±50 mg, ±75 mg, or ±100 mg);
   (iii) administering a first dose (C1D1) of ifosfamide on Day 3, a second dose (C1D2) of ifosfamide on Day 4, and a third dose (C1D3) of ifosfamide on Day 5, wherein the C1D1, the C1D2, and the C1D3 of ifosfamide are each about 3000 mg/m² (e.g., 3000 mg/m²±40 mg/m², ±50 mg/m², ±100 mg/m², ±200 mg/m², or ±300 mg/m²);
   (iv) administering a single dose (C1D1) of carboplatin on Day 3, wherein the C1D1 of carboplatin is about 635 mg/m² (e.g., 635 mg/m²±5 mg/m², ±10 mg/m², ±25 mg/m², ±50 mg/m², ±60 mg/m², or ±63.5 mg/m²); and
   (v) administering a first dose (C1D1) of etoposide on Day 3, a second dose (C1D2) of etoposide on Day 4, and a third dose (C1D3) of etoposide on Day 5, wherein the C1D1, the C1D2, and the C1D3 of etoposide are each about 100 mg/m² (e.g., 100 mg/m²±1 mg/m², ±2.5 mg/m², ±5 mg/m², or ±10 mg/m²); and
(b) the second dosing cycle comprises:
   (i) administering a single dose (C2D1) of glofitamab on Day 1, wherein the C2D1 of glofitamab is about 0.4 mg/kg (e.g., 0.4 mg/kg±0.005 mg/kg, ±0.01 mg/kg, ±0.02 mg/kg, ±0.03 mg/kg, or ±0.04 mg/kg), about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg), or about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg);
   (ii) administering a first dose (C2D1) of rituximab on Day 5, wherein the C2D1 of rituximab is about 375 mg/m² (e.g., 375 mg/m²±5 mg/m², ±10 mg/m², ±25 mg/m², or ±37.5 mg/m²);
   (iii) administering a first dose (C2D1) of ifosfamide on Day 6, a second dose (C2D2) of ifosfamide on Day 7, and a third dose (C2D3) of ifosfamide on Day 8, wherein the C2D1, the C2D2, and the C2D3 of ifosfamide are each about 3000 mg/m² (e.g., 3000 mg/m²±40 mg/m², ±50 mg/m², ±100 mg/m², ±200 mg/m², or ±300 mg/m²);
   (iv) administering a single dose (C2D1) of carboplatin on Day 6, wherein the C2D1 of carboplatin is about 5×(25+creatinine clearance) mg; and
   (v) administering a first dose (C2D1) of etoposide on Day 6, a second dose (C2D2) of etoposide on Day 7, and a third dose (C2D3) of etoposide on Day 8, wherein the C2D1, the C2D2, and the C2D3 of etoposide are each about 100 mg/m² (e.g., 100 mg/m²±1 mg/m², ±2.5 mg/m², ±5 mg/m², or ±10 mg/m²).

In one aspect, the invention features a method of treating a subject aged between 6 months and 17 years having a CD20-positive cell proliferative disorder comprising administering to the subject an effective amount of glofitamab, obinutuzumab, rituximab, ifosfamide, carboplatin, and etoposide in a dosing regimen comprising a first dosing cycle, a second dosing cycle, and a third dosing cycle, wherein
(a) the first dosing cycle comprises:
   (i) administering a first dose (C1D1) of glofitamab on Day 8 and a second dose (C1D2) of glofitamab on Day 15, wherein the C1D1 of glofitamab is about 0.03 mg/kg, about 0.04 mg/kg (e.g., 0.04 mg/kg±0.0005 mg/kg, ±0.001 mg/kg, ±0.002 mg/kg, ±0.003 mg/kg, or ±0.004 mg/kg), or about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 0.15 mg/kg (e.g., 0.15 mg/kg±0.001 mg/kg, ±0.0025 mg/kg, ±0.005 mg/kg, ±0.01 mg/kg, or ±0.015 mg/kg) or about 10 mg (e.g., 10 mg±0.05 mg, ±0.1 mg, ±0.2 mg, ±0.3 mg, ±0.5 mg, ±0.75 mg, or ±1 mg);
   (ii) administering a first dose (C1D1) of obinutuzumab on Day 1 and a second dose (C1D2) of obinutuzumab on Day 2, wherein the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab, and wherein the sum of the C1D1 and the C1D2 of obinutuzumab is about 38 mg/kg (e.g., 38 mg/kg±0.25 mg/kg, ±0.5 mg/kg, ±1 mg/kg, ±2 mg/kg, ±3 mg/kg, or ±3.8 mg/kg), about 28 mg/kg (e.g., 28 mg/kg±0.25 mg/kg, ±0.5 mg/kg, ±1 mg/kg, ±2 mg/kg, or ±2.8 mg/kg), about 23 mg/kg (e.g., 23 mg/kg±0.25 mg/kg, ±0.5 mg/kg, ±1 mg/kg, ±2 mg/kg, or ±2.3 mg/kg), about 20 mg/kg (e.g., 23 mg/kg±0.25 mg/kg, ±0.5 mg/kg, ±1 mg/kg, or ±2 mg/kg), or about 1000 mg (e.g., 1000 mg±5 mg, ±10 mg, ±20 mg, ±30 mg, ±50 mg, ±75 mg, or ±100 mg);
   (iii) administering a first dose (C1D1) of ifosfamide on Day 3, a second dose (C1D2) of ifosfamide on Day 4, and a third dose (C1D3) of ifosfamide on Day 5, wherein the C1D1, the C1D2, and the C1D3 of ifosfamide are each about 3000 mg/m² (e.g., 3000 mg/m²±40 mg/m², ±50 mg/m², ±100 mg/m², ±200 mg/m², or ±300 mg/m²);
   (iv) administering a single dose (C1D1) of carboplatin on Day 3, wherein the C1D1 of carboplatin is about 635 mg/m² (e.g., 635 mg/m²±5 mg/m², ±10 mg/m², ±25 mg/m², ±50 mg/m², ±60 mg/m², or ±63.5 mg/m²); and
   (v) administering a first dose (C1D1) of etoposide on Day 3, a second dose (C1D2) of etoposide on Day 4, and a third dose (C1D3) of etoposide on Day 5, wherein the C1D1, the C1D2, and the C1D3 of etoposide are each about 100 mg/m² (e.g., 100 mg/m²±1 mg/m², ±2.5 mg/m², ±5 mg/m², or ±10 mg/m²);
(b) the second dosing cycle comprises:
   (i) administering a single dose (C2D1) of glofitamab on Day 1, wherein the C2D1 of glofitamab is about 0.4 mg/kg (e.g., 0.4 mg/kg±0.005 mg/kg, ±0.01 mg/kg, ±0.02 mg/kg, ±0.03 mg/kg, or ±0.04 mg/kg), about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg), or about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg);
   (ii) administering a first dose (C2D1) of rituximab on Day 5, wherein the C2D1 of rituximab is about 375 mg/m² (e.g., 375 mg/m²±5 mg/m², ±10 mg/m², ±25 mg/m², or ±37.5 mg/m²);
   (iii) administering a first dose (C2D1) of ifosfamide on Day 6, a second dose (C2D2) of ifosfamide on Day 7, and a third dose (C2D3) of ifosfamide on Day 8, wherein the C2D1, the C2D2, and the C2D3 of ifosfamide are each about 3000 mg/m² (e.g., 3000 mg/m²±40 mg/m², ±50 mg/m², ±100 mg/m², ±200 mg/m², or ±300 mg/m²);
   (iv) administering a single dose (C2D1) of carboplatin on Day 6, wherein the C2D1 of carboplatin is about 5×(25+creatinine clearance) mg; and
   (v) administering a first dose (C2D1) of etoposide on Day 6, a second dose (C2D2) of etoposide on Day 7, and a third dose (C2D3) of etoposide on Day 8, wherein the C2D1, the C2D2, and the C2D3 of etoposide are each about 100 mg/m² (e.g., 100 mg/m²±1 mg/m², ±2.5 mg/m², ±5 mg/m², or ±10 mg/m²); and
(c) the third dosing cycle comprises:
   (i) administering a single dose (C3D1) of glofitamab on Day 1, wherein the C3D1 of glofitamab is about 0.4 mg/kg (e.g., 0.4 mg/kg±0.005 mg/kg, ±0.01 mg/kg, ±0.02 mg/kg, ±0.03 mg/kg, or ±0.04 mg/kg), about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg), or about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg);
   (ii) administering a first dose (C3D1) of rituximab on Day 5, wherein the C3D1 of rituximab is about 375 mg/m² (e.g., 375 mg/m²±5 mg/m², ±10 mg/m², ±25 mg/m², or ±37.5 mg/m²);
   (iii) administering a first dose (C3D1) of ifosfamide on Day 6, a second dose (C3D2) of ifosfamide on Day 7, and a third dose (C3D3) of ifosfamide on Day 8, wherein the C3D1, the C3D2, and the C3D3 of ifosfamide are each about 3000 mg/m² (e.g., 3000 mg/m²±40 mg/m², ±50 mg/m², ±100 mg/m², ±200 mg/m², or ±300 mg/m²);
   (iv) administering a single dose (C3D1) of carboplatin on Day 6, wherein the C3D1 of carboplatin is about 5×(25+creatinine clearance) mg; and
   (v) administering a first dose (C3D1) of etoposide on Day 6, a second dose (C3D2) of etoposide on Day 7, and a third dose (C3D3) of etoposide on Day 8, wherein the C3D1, the C3D2, and the C3D3 of etoposide are each about 100 mg/m² (e.g., 100 mg/m²±1 mg/m², ±2.5 mg/m², ±5 mg/m², or ±10 mg/m²).

In one embodiment, mesna is to be administered to the subject on Days 3, 4, and 5 of the first dosing cycle, on Days 6, 7, and 8 of the second dosing cycle, and/or on Days 6, 7, and 8 of each additional dosing cycle. In one embodiment, mesna is to be administered intravenously daily as five doses totaling 3000 mg/m² in amount. In one embodiment, mesna is to be administered intravenously at a first dose of about 600 mg/m² prior to the administration of any dose of ifosfamide and at four repeated doses of about 600 mg/m² each at about three hours, about six hours, about nine hours, and about 12 hours, respectively, after the first dose of ifosfamide.

In one aspect, the invention features a method of treating a subject aged between 18 years and 30 years having a CD20-positive cell proliferative disorder comprising administering to the subject an effective amount of glofitamab, obinutuzumab, rituximab, ifosfamide, carboplatin, and etoposide in a dosing regimen comprising at least a first dosing cycle and a second dosing cycle, wherein
(a) the first dosing cycle comprises administering a first dose (C1D1) of glofitamab on Day 8 and administering a second dose (C1D2) of glofitamab on Day 15, wherein the C1D1 of glofitamab is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 10 mg (e.g., 10 mg±0.05 mg, ±0.1 mg, ±0.2 mg, ±0.3 mg, ±0.5 mg, ±0.75 mg, or ±1 mg); and
(b) the second dosing cycle comprises administering a single dose (C2D1) of glofitamab on Day 1, wherein the C2D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one aspect, the invention features a method of treating a subject aged between 18 years and 30 years having a CD20-positive cell proliferative disorder comprising administering to the subject an effective amount of glofitamab, obinutuzumab, rituximab, ifosfamide, carboplatin, and etoposide in a dosing regimen comprising a first dosing cycle, a second dosing cycle, and a third dosing cycle, wherein
(a) the first dosing cycle comprises administering a first dose (C1D1) of glofitamab on Day 8 and administering a second dose (C1D2) of glofitamab on Day 15, wherein the C1D1 of glofitamab is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 10 mg (e.g., 10 mg±0.05 mg, ±0.1 mg, ±0.2 mg, ±0.3 mg, ±0.5 mg, ±0.75 mg, or ±1 mg);
(b) the second dosing cycle comprises administering a single dose (C2D1) of glofitamab on Day 1, wherein the C2D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or 3 mg); and
(c) the third dosing cycle comprises administering a single dose (C3D1) of glofitamab on Day 1, wherein the C3D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one aspect, the invention features a method of treating a subject aged between 18 years and 30 years having a CD20-positive cell proliferative disorder comprising administering to the subject an effective amount of glofitamab, obinutuzumab, rituximab, ifosfamide, carboplatin, and etoposide in a dosing regimen comprising at least a first dosing cycle and a second dosing cycle, wherein
(a) the first dosing cycle comprises:
   (i) administering a first dose (C1D1) of glofitamab on Day 8 and a second dose (C1D2) of glofitamab on Day 15, wherein the C1D1 of glofitamab is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 10 mg (e.g., 10 mg±0.05 mg, ±0.1 mg, ±0.2 mg, ±0.3 mg, ±0.5 mg, ±0.75 mg, or ±1 mg);
   (ii) administering a first dose (C1D1) of obinutuzumab on Day 1 and a second dose (C1D2) of obinutuzumab on Day 2, wherein the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab, and wherein the sum of the C1D1 and the C1D2 of obinutuzumab is about 1000 mg (e.g., 1000 mg±5 mg, ±10 mg, ±20 mg, ±30 mg, ±50 mg, ±75 mg, or 100 mg);
   (iii) administering a single dose (C1D1) of ifosfamide on Day 3, wherein the C1D1 of ifosfamide is about 5000 mg/m² (e.g., 5000 mg/m²±50 mg/m², ±100 mg/m², ±200 mg/m², ±300 mg/m², ±400 mg/m², or ±500 mg/m²) with a maximum dose of about 800 mg (e.g., 800 mg±10 mg, ±25 mg, ±50 mg, or ±80 mg);
   (iv) administering a single dose (C1D1) of carboplatin on Day 3, wherein the C1D1 of carboplatin is about 5×(25+creatinine clearance) mg; and
   (v) administering a first dose (C1D1) of etoposide on Day 3, a second dose (C1D2) of etoposide on Day 4, and a third dose (C1D3) of etoposide on Day 5, wherein the C1D1, the C1D2, and the C1D3 of etoposide are each about 100 mg/m² (e.g., 100 mg/m²±1 mg/m², ±2.5 mg/m², ±5 mg/m², or ±10 mg/m²); and
(b) the second dosing cycle comprises:
   (i) administering a single dose (C2D1) of glofitamab on Day 1, wherein the C2D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg);
   (ii) administering a first dose (C2D1) of rituximab on Day 5, wherein the C2D1 of rituximab is about 375 mg/m² (e.g., 375 mg/m²±5 mg/m², ±10 mg/m², ±25 mg/m², or ±37.5 mg/m²);
   (iii) administering a single dose (C2D1) of ifosfamide on Day 6, wherein the C2D1 of ifosfamide is about 5000 mg/m² (e.g., 5000 mg/m²±50 mg/m², ±100 mg/m², ±200 mg/m², ±300 mg/m², ±400 mg/m², or ±500 mg/m²) with a maximum dose of about 800 mg (e.g., 800 mg±10 mg, ±25 mg, ±50 mg, or ±80 mg);
   (iv) administering a single dose (C2D1) of carboplatin on Day 6, wherein the C2D1 of carboplatin is about 5×(25+creatinine clearance) mg; and
   (v) administering a first dose (C2D1) of etoposide on Day 6, a second dose (C2D2) of etoposide on Day 7, and a third dose (C2D3) of etoposide on Day 8, wherein the C2D1, the C2D2, and the C2D3 of etoposide are each about 100 mg/m² (e.g., 100 mg/m²±1 mg/m², ±2.5 mg/m², ±5 mg/m², or ±10 mg/m²).

In one aspect, the invention features a method of treating a subject aged between 18 years and 30 years having a CD20-positive cell proliferative disorder comprising administering to the subject an effective amount of glofitamab, obinutuzumab, rituximab, ifosfamide, carboplatin, and etoposide in a dosing regimen comprising a first dosing cycle, a second dosing cycle, and a third dosing cycle, wherein
(a) the first dosing cycle comprises:
   (i) administering a first dose (C1D1) of glofitamab on Day 8 and a second dose (C1D2) of glofitamab on Day 15, wherein the C1D1 of glofitamab is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 10 mg (e.g., 10 mg±0.05 mg, ±0.1 mg, ±0.2 mg, ±0.3 mg, ±0.5 mg, ±0.75 mg, or ±1 mg);
   (ii) administering a first dose (C1D1) of obinutuzumab on Day 1 and a second dose (C1D2) of obinutuzumab on Day 2, wherein the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab, and wherein the sum of the C1D1 and the C1D2 of obinutuzumab is about 1000 mg (e.g., 1000 mg±5 mg, ±10 mg, ±20 mg, ±30 mg, ±50 mg, ±75 mg, or 100 mg);
   (iii) administering a single dose (C1D1) of ifosfamide on Day 3, wherein the C1D1 of ifosfamide is about 5000 mg/m² (e.g., 5000 mg/m²±50 mg/m², ±100 mg/m², ±200 mg/m², ±300 mg/m², ±400 mg/m², or ±500 mg/m²) with a maximum dose of about 800 mg (e.g., 800 mg±10 mg, ±25 mg, ±50 mg, or ±80 mg);
   (iv) administering a single dose (C1D1) of carboplatin on Day 3, wherein the C1D1 of carboplatin is about 5×(25+creatinine clearance) mg; and
   (v) administering a first dose (C1D1) of etoposide on Day 3, a second dose (C1D2) of etoposide on Day 4, and a third dose (C1D3) of etoposide on Day 5, wherein the C1D1, the C1D2, and the C1D3 of etoposide are each about 100 mg/m² (e.g., 100 mg/m²±1 mg/m², ±2.5 mg/m², ±5 mg/m², or ±10 mg/m²);
(b) the second dosing cycle comprises:
   (i) administering a single dose (C2D1) of glofitamab on Day 1, wherein the C2D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg);
   (ii) administering a first dose (C2D1) of rituximab on Day 5, wherein the C2D1 of rituximab is about 375 mg/m² (e.g., 375 mg/m²±5 mg/m², ±10 mg/m², ±25 mg/m², or ±37.5 mg/m²);
   (iii) administering a single dose (C2D1) of ifosfamide on Day 6, wherein the C2D1 of ifosfamide is about 5000 mg/m² (e.g., 5000 mg/m²±50 mg/m², ±100 mg/m², ±200 mg/m², ±300 mg/m², ±400 mg/m², or ±500 mg/m²) with a maximum dose of about 800 mg (e.g., 800 mg±10 mg, ±25 mg, ±50 mg, or ±80 mg);
   (iv) administering a single dose (C2D1) of carboplatin on Day 6, wherein the C2D1 of carboplatin is about 5×(25+creatinine clearance) mg; and
   (v) administering a first dose (C2D1) of etoposide on Day 6, a second dose (C2D2) of etoposide on Day 7, and a third dose (C2D3) of etoposide on Day 8, wherein the C2D1, the C2D2, and the C2D3 of etoposide are each about 100 mg/m² (e.g., 100 mg/m²±1 mg/m², ±2.5 mg/m², ±5 mg/m², or ±10 mg/m²); and
(c) the third dosing cycle comprises:
   (i) administering a single dose (C3D1) of glofitamab on Day 1, wherein the C3D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg);
   (ii) administering a first dose (C3D1) of rituximab on Day 5, wherein the C3D1 of rituximab is about 375 mg/m² (e.g., 375 mg/m²±5 mg/m², ±10 mg/m², ±25 mg/m², or ±37.5 mg/m²);
   (iii) administering a single dose (C3D1) of ifosfamide on Day 6, wherein the C3D1 of ifosfamide is about 5000 mg/m² (e.g., 5000 mg/m²±50 mg/m², ±100 mg/m², ±200 mg/m², ±300 mg/m², ±400 mg/m², or ±500 mg/m²) with a maximum dose of about 800 mg (e.g., 800 mg±10 mg, ±25 mg, ±50 mg, or ±80 mg);
   (iv) administering a single dose (C3D1) of carboplatin on Day 6, wherein the C3D1 of carboplatin is about 5×(25+creatinine clearance) mg; and
   (v) administering a first dose (C3D1) of etoposide on Day 6, a second dose (C3D2) of etoposide on Day 7, and a third dose (C3D3) of etoposide on Day 8, wherein the C3D1, the C3D2, and the C3D3 of etoposide are each about 100 mg/m² (e.g., 100 mg/m²±1 mg/m², ±2.5 mg/m², ±5 mg/m², or ±10 mg/m²).

In one embodiment, mesna is to be administered simultaneously with any dose of ifosfamide. In one embodiment, mesna is to be administered intravenously at a dose of about 5000 mg/m² (e.g., 5000 mg/m²±50 mg/m², ±100 mg/m², ±200 mg/m², ±300 mg/m², ±400 mg/m², or ±500 mg/m²). In one embodiment, mesna is to be administered via continuous infusion over about 24 hours on Day 3 of the first dosing cycle, on Day 6 of the second dosing cycle, and/or on Day 6 of each additional dosing cycle. In one embodiment, the CD20-positive cell proliferative disorder is a relapsed and/or refractory DLBCL. In one embodiment, the CD20-positive cell proliferative disorder is a relapsed and/or refractory mature B cell NHL.

In one embodiment, the bispecific antibody that binds to CD20 and CD3 comprises at least one Fab molecule which specifically binds to CD20 comprising the following six hypervariable regions (HVRs):
(i) an HVR-H1 comprising the amino acid sequence of YSWIN (SEQ ID NO: 1);
(ii) an HVR-H2 comprising the amino acid sequence of RIFPGDGDTDYNGKFKG (SEQ ID NO: 2);
(iii) an HVR-H3 comprising the amino acid sequence of NVFDGYWLVY (SEQ ID NO:3);
(iv) an HVR-L1 comprising the amino acid sequence of RSSKSLLHSNGITYLY (SEQ ID NO: 4);
(v) an HVR-L2 comprising the amino acid sequence of QMSNLVS (SEQ ID NO: 5); and
(vi) an HVR-L3 comprising the amino acid sequence of AQNLELPYT (SEQ ID NO: 6).

In one embodiment, the bispecific antibody that binds to CD20 and CD3 comprises at least one Fab molecule which specifically binds to CD20 comprising (a) a heavy chain variable (VH) domain comprising an amino acid sequence having at least 95% sequence identity to the amino acid sequence of SEQ ID NO: 7; (b) a light chain variable (VL) domain comprising an amino acid sequence having at least 95% sequence identity to the amino acid sequence of SEQ ID NO: 8; or (c) a VH domain as in (a) and a VL domain as in (b). In one embodiment, the Fab molecule which specifically binds to CD20 comprises (a) a VH domain comprising an amino acid sequence of SEQ ID NO: 7 and (b) a VL domain comprising an amino acid sequence of SEQ ID NO: 8.

In one embodiment, the bispecific antibody that binds to CD20 and CD3 comprises at least one Fab molecule which specifically binds to CD3 comprising the following six HVRs:
(i) an HVR-H1 comprising the amino acid sequence of TYAMN (SEQ ID NO: 9);
(ii) an HVR-H2 comprising the amino acid sequence of RIRSKYNNYATYYADSVKG (SEQ ID NO: 10);
(iii) an HVR-H3 comprising the amino acid sequence of HGNFGNSYVSWFAY (SEQ ID NO: 11);
(iv) an HVR-L1 comprising the amino acid sequence of GSSTGAVTTSNYAN (SEQ ID NO: 12);
(v) an HVR-L2 comprising the amino acid sequence of GTNKRAP (SEQ ID NO: 13); and
(vi) an HVR-L3 comprising the amino acid sequence of ALWYSNLWV (SEQ ID NO: 14).

In one embodiment, the bispecific antibody that binds to CD20 and CD3 comprises at least one Fab molecule which specifically binds to CD3 comprising (a) a heavy chain variable (VH) domain comprising an amino acid sequence having at least 95% sequence identity to the amino acid sequence of SEQ ID NO: 15; (b) a light chain variable (VL) domain comprising an amino acid sequence having at least 95% sequence identity to the amino acid sequence of SEQ ID NO: 16; or (c) a VH domain as in (a) and a VL domain as in (b).

In one embodiment, the Fab molecule which specifically binds to CD3 comprises (a) a VH domain comprising an amino acid sequence of SEQ ID NO: 15 and (b) a VL domain comprising an amino acid sequence of SEQ ID NO: 16.

In one embodiment, the bispecific antibody that binds to CD20 and CD3 is bivalent for CD20 and monovalent for CD3. In one embodiment, the bispecific antibody that binds to CD20 and CD3 comprises two Fab molecules which specifically bind to CD20 and one Fab molecule which specifically binds to CD3.

In one embodiment, the bispecific antibody that binds to CD20 and CD3 is a humanized antibody. In one embodiment, the bispecific antibody that binds to CD20 and CD3 is glofitamab.

In one embodiment, the bispecific antibody that binds to CD20 and CD3 is administered intravenously.

In one embodiment, the anti-CD20 antibody is administered intravenously.

In one embodiment, the CD20-positive cell proliferative disorder is a B cell proliferative disorder. In one embodiment, the B cell proliferative disorder is a non-Hodgkin's lymphoma (NHL) or a central nervous system lymphoma (CNSL). In one embodiment, the NHL is a diffuse-large B cell lymphoma (DLBCL), a follicular lymphoma (FL), a mantle cell lymphoma (MCL), a marginal zone lymphoma (MZL), a high-grade B cell lymphoma, a primary mediastinal (thymic) large B cell lymphoma (PMLBCL), a diffuse B cell lymphoma, or a small lymphocytic lymphoma. In one embodiment, the NHL is a Burkitt lymphoma (BL) or a Burkitt leukemia (BAL). In one embodiment, the NHL is aggressive and/or mature. In one embodiment, the NHL is relapsed and/or refractory. In one embodiment, the B cell proliferative disorder is a relapsed and/or refractory mature B cell NHL. In one embodiment, the subject has received no more than one prior systemic therapy.

In one embodiment, the prior systemic therapy comprises an anti-CD20 antibody and an anthracycline. In one embodiment, the subject is human. In one embodiment, the subject is transplant or CAR-T cell therapy eligible. In one embodiment, the subject receives autologous stem cell transplantation (ASCT) after completion of the dosing regimen of the method described above. In one embodiment, the ASCT is an autologous hematopoietic stem cell transplant. In one embodiment, the subject receives allogenic hematopoietic stem cell transplant after completion of the dosing regimen as described above. In one embodiment, the subject receives CAR-T cell therapy after completion of the dosing regimen as described above.

A further aspect of the present invention relates to the invention as described herein.

Each and every embodiment can be combined unless the context clearly suggests otherwise. Each and every embodiment can be applied to each and every aspect of the invention unless the context clearly suggests otherwise.

Specific embodiments of the present invention will become evident from the following more detailed description of certain preferred embodiments and the claims.

### BRIEF DESCRIPTION OF THE DRAWINGS

FIG. 1A-FIG. 1N are schematic diagrams showing configurations of exemplary anti-CD20/anti-CD3 bispecific antibodies.

FIG. 2 is a schematic diagram showing the structure of glofitamab.

FIG. 3 is a schematic showing the overview of the study design as described in Example 1. ASCT=autologous stem cell transplantation; CAR-T=Chimeric Antigen Receptor T-cells; CR=Complete Response; DLBCL=diffuse large B-cell lymphoma; EOT=end of treatment; Glofit-R-ICE=Glofitamab in combination with Rituximab plus Ifosfamide, Carboplatin, Etoposide; Gpt=Obinutuzumab (GAZYVA®) pretreatment; PD=progressive disease; PR=partial response; R/R=relapsed or refractory; SD=stable disease. ^{a}: 2-3 cycles depending on institutional standard. ^{b}: ASCT conditioning regimen and supportive care per institutional standard.

FIG. 4 is a schematic showing the dosing schema of the study as described in Example 1. D=Day. ^{a}: Carboplatin dose in mg to target AUC of 5 mg/mL/min.

FIG. 5 is a schematic showing the study schema of Parts 1 and 2 in Example 2. B-NHL=B-cell non-Hodgkin lymphoma; Glofit=glofitamab; Ped=pediatric; PK=pharmacokinetic; R-ICE=rituximab, ifosfamide, carboplatin, and etoposide; Rec=recommended; R/R=relapsed/refractory; RR=response rate. In Part 1 of the study, a safety run-in with starting dose is based on population PK modelling and matching adult exposures with step-up dosing. Safety and pharmacokinetics are evaluated from at least 3 participants until the recommended pediatric Part 2 dose is declared. At least 10 pediatric participants are evaluated for early efficacy at the recommended pediatric dose before Part 2 can commence. During Part 2 of the study, enrollment is open to include young adult participants up to 30 years old (inclusive), but they do not count towards the sample size of 45.

FIG. 6 is a schematic showing the dosing schema for glofitamab in combination with R-ICE described in Example 2. C=Cycle; CR=complete response; D=Day; G=obinutuzumab; CR=complete response; FD=full dose; G-CSF=granulocyte colony-stimulating factor; Glofit=glofitamab; GpT=obinutuzumab pretreatment; HSCT=hematopoietic stem cell transplantation; ICE=ifosfamide, carboplatin, and etoposide; PD=progressive disease; R-ICE=rituximab plus ifosfamide, carboplatin, etoposide; SUD=step-up dose. ^{a}Obinutuzumab pretreatment will be split into one-tenth of the full dose on Day 1 of the first dosing cycle (Cycle 1) and nine-tenth of the full dose on Day 2 of Cycle 1.

FIG. 7 is a schematic showing the treatment administration schedule of pediatric patients (aged 6 months to 17 years) in Example 2. *: The arrows indicate mandatory initiation of granulocyte colony-stimulating factor (G-CSF) therapy. **: Participants with CNS disease, with any histology, receive intrathecal (IT) therapy on Days 3, 10, and 17 of Cycle 1 and Days 5, 12, and 19 of Cycles 2 and 3. Participants with large B-cell lymphoma who are CNS negative receive IT therapy on Day 3 of Cycle 1 only. A single dose of obinutuzumab pretreatment is split into one-tenth of the full dose on Day 1 of Cycle 1 and nine-tenth of the full dose on Day 2 of Cycle 1. Participants with Burkitt lymphoma (BL) who are CNS negative receive IT therapy on Day 3 of Cycle 1 and Day 5 of Cycles 2 and 3. Glofitamab is given as two step-up doses on Day 8 and Day 15 of Cycle 1 and as full doses on Day 1 of Cycles 2 and 3.

FIG. 8 is a schematic showing the treatment administration schedule of young adult patients (aged 18 years to 30 years) in Example 2. *: The arrows indicate mandatory initiation of G-CSF therapy. **: Participants with CNS disease, with any histology, receive IT therapy on Days 3, 10, and 17 of Cycle 1 and Days 5, 12, and 19 of Cycles 2 and 3. Participants with large B-cell lymphoma who are CNS negative receive IT therapy on Day 3 of Cycle 1 only. A single dose of obinutuzumab pretreatment is split into one-tenth of the full dose on Day 1 of Cycle 1 and nine-tenth of the full dose on Day 2 of Cycle 1. Participants with BL who are CNS negative receive IT therapy on Day 3 of Cycle 1 and Day 5 of Cycles 2 and 3. G-CSF=granulocyte colony-stimulating factor. Glofitamab is given as two step-up doses on Day 8 and Day 15 of Cycle 1 and as full doses on Day 1 of Cycles 2 and 3.

### DETAILED DESCRIPTION OF THE INVENTION

The invention provides methods for treating a subject having a CD20-positive cell proliferative disorder (e.g., a B cell proliferative disorder (e.g., non-Hodgkin's lymphoma (NHL) (e.g., a relapsed and/or refractory NHL, a diffuse-large B cell lymphoma (DLBCL) (e.g., a relapsed and/or refractory DLBCL), a follicular lymphoma (FL) (e.g., a relapsed and/or refractory FL or a transformed FL), or a mantle cell lymphoma (MCL) (e.g., a relapsed or refractory MCL)), or a central nervous system lymphoma (CNSL))) that includes administering to the subject an anti-CD20/anti-CD3 bispecific antibody in combination with an anti-CD20 antibody and one or more chemotherapeutic agents selected from ifosfamide, carboplatin and/or etoposide.

### (i) General Techniques

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001).

### (ii) Definitions

Terms are used herein as generally used in the art, unless otherwise defined in the following.

The term “cluster of differentiation 20” or “CD20” as used herein, refers to any native CD20 from any vertebrate source, including mammals such as primates (e.g., humans) and rodents (e.g., mice and rats), unless otherwise indicated. CD20 (also known as B-lymphocyte antigen CD20, B-lymphocyte surface antigen B1, Leu-16, Bp35, BMS, and LFS; the human protein is characterized in UniProt database entry P11836) is a hydrophobic transmembrane protein with a molecular weight of approximately 35 kD expressed on pre-B and mature B lymphocytes (Valentine, M. A. et al., J. Biol. Chem. 264 (1989) 11282-11287; Tedder, T. F., et al., Proc. Natl. Acad. Sci. U.S.A. 85 (1988) 208-212; Stamenkovic, I., et al., J. Exp. Med. 167 (1988) 1975-1980; Einfeld, D. A., et al., EMBO J. 7 (1988) 711-717; Tedder, T. F., et al., J. Immunol. 142 (1989) 2560-2568). The corresponding human gene is Membrane-spanning 4-domains, subfamily A, member 1, also known as MS4A1. This gene encodes a member of the membrane-spanning 4A gene family. Members of this nascent protein family are characterized by common structural features and similar intron/exon splice boundaries and display unique expression patterns among hematopoietic cells and nonlymphoid tissues. This gene encodes the B-lymphocyte surface molecule which plays a role in the development and differentiation of B-cells into plasma cells. This family member is localized to 11q12, among a cluster of family members. The term encompasses “full-length,” unprocessed CD20 as well as any form of CD20 that results from processing in the cell. The term also encompasses naturally occurring variants of CD20, e.g., splice variants or allelic variants. Alternative splicing of this gene results in two transcript variants which encode the same protein. In one embodiment, CD20 is human CD20.

The terms “anti-CD20 antibody” and “an antibody that binds to CD20” refer to an antibody that is capable of binding CD20 with sufficient affinity such that the antibody is useful as a diagnostic and/or therapeutic agent in targeting CD20. In one embodiment, the extent of binding of an anti-CD20 antibody to an unrelated, non-CD20 protein is less than about 10% of the binding of the antibody to CD20 as measured, e.g., by a radioimmunoassay (RIA). In certain embodiments, an antibody that binds to CD20 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁸ M to 10⁻¹³ M). In certain embodiments, an anti-CD20 antibody binds to an epitope of CD20 that is conserved among CD20 from different species.

By “Type II anti-CD20 antibody” is meant an anti-CD20 antibody having binding properties and biological activities of Type II anti-CD20 antibodies as described in Cragg et al., Blood 103 (2004) 2738-2743; Cragg et al., Blood 101 (2003) 1045-1052, Klein et al., mAbs 5 (2013), 22-33, and summarized in Table 1 below.

TABLE 1
Properties of type I and type II anti-CD20 antibodies

| type I anti-CD20 antibodies | type II anti-CD20 antibodies |
|---|---|
| Bind class I CD20 epitope | Bind class II CD20 epitope |
| Localize CD20 to lipid rafts | Do not localize CD20 to lipid rafts |
| High CDC * | Low CDC * |
| ADCC activity * | ADCC activity * |
| Full binding capacity to B cells | Approx. half binding capacity to B cells |
| Weak homotypic aggregation | Homotypic aggregation |
| Low cell death induction | Strong cell death induction |

* if IgG₁ isotype

Examples of type II anti-CD20 antibodies include, e.g., obinutuzumab (GA101), tositumumab (B1), humanized B-Ly1 antibody IgG1 (a chimeric humanized IgG1 antibody as disclosed in WO 2005/044859), 11B8 IgG1 (as disclosed in WO 2004/035607) and AT80 IgG1.

Examples of type I anti-CD20 antibodies include, e.g., rituximab, ofatumumab, veltuzumab, ocaratuzumab, ocrelizumab, PRO131921, ublituximab, HI47 IgG3 (ECACC, hybridoma), 2C6 IgG1 (as disclosed in WO 2005/103081), 2F2 IgG1 (as disclosed in WO 2004/035607 and WO 2005/103081) and 2H7 IgG1 (as disclosed in WO 2004/056312).

“CD3” refers to any native CD3 from any vertebrate source, including mammals such as primates (e.g., humans), non-human primates (e.g., cynomolgus monkeys) and rodents (e.g., mice and rats), unless otherwise indicated. The term encompasses “full-length,” unprocessed CD3 as well as any form of CD3 that results from processing in the cell. The term also encompasses naturally occurring variants of CD3, e.g., splice variants or allelic variants. In one embodiment, CD3 is human CD3, particularly the epsilon subunit of human CD3 (CD3ε). The amino acid sequence of human CD3c is shown in UniProt (www.uniprot.org) accession no. P07766 (version 144), or NCBI (www.ncbi.nlm.nih.gov/) RefSeq NP_000724.1. The amino acid sequence of cynomolgus monkey [Macaca fascicularis] CD3ε is shown in NCBI GenBank no. BAB71849.1.

The terms “anti-CD20/anti-CD3 bispecific antibody” and “a bispecific antibody that binds to CD20 and CD3” refer to a bispecific antibody that is capable of binding both CD20 and CD3 with sufficient affinity such that the antibody is useful as a diagnostic and/or therapeutic agent in targeting CD20 and/or CD3. In one embodiment, the extent of binding of a bispecific antibody that binds to CD20 and CD3 to an unrelated, non-CD3 protein and/or non-CD20 protein is less than about 10% of the binding of the antibody to CD3 and/or CD20 as measured, e.g., by a radioimmunoassay (RIA). In certain embodiments, a bispecific antibody that binds to CD20 and CD3 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain embodiments, a bispecific antibody that binds to CD20 and CD3 binds to an epitope of CD3 that is conserved among CD3 from different species and/or an epitope of CD20 that is conserved among CD20 from different species. One example of an anti-CD20/anti-CD3 bispecific antibody is glofitamab (WHO Drug Information (International Nonproprietary Names for Pharmaceutical Substances), Recommended INN: List 83, 2020, vol. 34, no. 1, p. 39; Proposed INN: List 121 WHO Drug Information, Vol. 33, No. 2, 2019, page 276, also known as CD20-TCB, R07082859, or RG6026; CAS #: 2229047-91-8).

As used herein, the term “release of cytokines” or “cytokine release” is synonymous with “cytokine storm” or “cytokine release syndrome” (abbreviated as “CRS”), and refers to an increase in the levels of cytokines, particularly tumor necrosis factor alpha (TNF-α), interferon gamma (IFN-γ), interleukin-6 (IL-6), interleukin-10 (IL-10), interleukin-2 (IL-2) and/or interleukin-8 (IL-8), in the blood of a subject during or shortly after (e.g., within 1 day of) administration of a therapeutic agent, resulting in adverse symptoms. Cytokine release is defined as a supraphysiologic response following administration of any immune therapy that results in activation or engagement of endogenous or infused T cells and/or other immune effector cells. Symptoms can be progressive, always include fever at the onset, and may include hypotension, capillary leak (hypoxia), and end-organ dysfunction (Lee et al. 2019). In some instances, e.g., after the administration of CAR-T cells, CRS can also occur several days after administration upon expansion of the CAR-T cells. The incidence and severity typically decrease with subsequent infusions. Symptoms may range from symptomatic discomfort to fatal events, and may include fever, chills, dizziness, hypertension, hypotension, dyspnea, restlessness, sweating, flushing, skin rash, tachycardia, tachypnea, headache, tumor pain, nausea, vomiting and/or organ failure.

The term “amino acid mutation” as used herein is meant to encompass amino acid substitutions, deletions, insertions, and modifications. Any combination of substitution, deletion, insertion, and modification can be made to arrive at the final construct, provided that the final construct possesses the desired characteristics, e.g., reduced binding to an Fc receptor. Amino acid sequence deletions and insertions include amino- and/or carboxy-terminal deletions and insertions of amino acids. Particular amino acid mutations are amino acid substitutions. For the purpose of altering, e.g., the binding characteristics of an Fc region, non-conservative amino acid substitutions, i.e., replacing one amino acid with another amino acid having different structural and/or chemical properties, are particularly preferred. Amino acid substitutions include replacement by non-naturally occurring amino acids or by naturally occurring amino acid derivatives of the twenty standard amino acids (e.g., 4-hydroxyproline, 3-methylhistidine, ornithine, homoserine, 5-hydroxylysine). Amino acid mutations can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful. Various designations may be used herein to indicate the same amino acid mutation. For example, a substitution from proline at position 329 of the Fc region to glycine can be indicated as 329G, G329, G₃₂₉, P329G, or Pro329Gly.

“Affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity which reflects a 1:1 interaction between members of a binding pair (e.g., receptor and a ligand). The affinity of a molecule X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by well-established methods known in the art. A particular method for measuring affinity is Surface Plasmon Resonance (SPR).

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

As used herein, the term “antigen binding moiety” refers to a polypeptide molecule that specifically binds to an antigenic determinant. In one embodiment, an antigen binding moiety is able to direct the entity to which it is attached (e.g., a cytokine or a second antigen binding moiety) to a target site, for example to a specific type of tumor cell or tumor stroma bearing the antigenic determinant. Antigen binding moieties include antibodies and fragments thereof as further defined herein. Preferred antigen binding moieties include an antigen binding domain of an antibody, comprising an antibody heavy chain variable region and an antibody light chain variable region. In certain embodiments, the antigen binding moieties may include antibody constant regions as further defined herein and known in the art. Useful heavy chain constant regions include any of the five isotypes: α, δ, ε, γ, or μ. Useful light chain constant regions include any of the two isotypes: κ and λ.

By “binds,” “specifically binds,” or is “specific for” is meant that the binding is selective for the antigen and can be discriminated from unwanted or non-specific interactions. The ability of an antigen binding moiety to bind to a specific antigenic determinant can be measured either through an enzyme-linked immunosorbent assay (ELISA) or other techniques familiar to one of skill in the art, e.g., surface plasmon resonance technique (analyzed on a BIAcore instrument) (Liljeblad et al., Glyco J. 17, 323-329 (2000)), and traditional binding assays (Heeley, Endocr Res. 28, 217-229 (2002)). In one embodiment, the extent of binding of an antigen binding moiety to an unrelated protein is less than about 10% of the binding of the antigen binding moiety to the antigen as measured, e.g., by SPR. In certain embodiments, an antigen binding moiety that binds to the antigen, or an antigen binding molecule comprising that antigen binding moiety, has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M).

“Reduced binding,” for example reduced binding to an Fc receptor, refers to a decrease in affinity for the respective interaction, as measured for example by SPR. For clarity the term includes also reduction of the affinity to zero (or below the detection limit of the analytic method), i.e., complete abolishment of the interaction. Conversely, “increased binding” refers to an increase in binding affinity for the respective interaction.

As used herein, the term “antigen binding molecule” refers in its broadest sense to a molecule that specifically binds an antigenic determinant. Examples of antigen binding molecules are immunoglobulins and derivatives, e.g., fragments, thereof.

As used herein, the term “antigenic determinant” is synonymous with “antigen” and “epitope,” and refers to a site (e.g., a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, free in blood serum, and/or in the extracellular matrix (ECM). The proteins referred to as antigens herein (e.g., CD3) can be any native form the proteins from any vertebrate source, including mammals such as primates (e.g., humans) and rodents (e.g., mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g., splice variants or allelic variants. An exemplary human protein useful as antigen is CD3, particularly the epsilon subunit of CD3 (see UniProt no. P07766 (version 130), NCBI RefSeq no. NP_000724.1, for the human sequence; or UniProt no. Q95LI5 (version 49), NCBI GenBank no. BAB71849.1, for the cynomolgus [Macaca fascicularis] sequence). In certain embodiments a T cell activating bispecific antigen binding molecule described herein binds to an epitope of CD3 or a target cell antigen that is conserved among the CD3 or target cell antigen from different species.

As used herein, term “polypeptide” refers to a molecule composed of monomers (amino acids) linearly linked by amide bonds (also known as peptide bonds). The term “polypeptide” refers to any chain of two or more amino acids, and does not refer to a specific length of the product. Thus, peptides, dipeptides, tripeptides, oligopeptides, “protein,” “amino acid chain,” or any other term used to refer to a chain of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded.

By an “isolated” polypeptide or a variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for the purpose of the invention, as are native or recombinant polypeptides which have been separated, fractionated, or partially or substantially purified by any suitable technique.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y
where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen binding activity.

The terms “full length antibody,” “intact antibody,” and “whole antibody” are used herein interchangeably to refer to an antibody having a structure substantially similar to a native antibody structure or having heavy chains that contain an Fc region as defined herein.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab ′)₂, diabodies, linear antibodies, single-chain antibody molecules (e.g., scFv), and multispecific antibodies formed from antibody fragments. The term “antibody fragment” as used herein also encompasses single-domain antibodies.

The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a constant light (CL) domain, also called a light chain constant region. The heavy chain of an immunoglobulin may be assigned to one of five classes, called α (IgA), δ (IgD), (IgE), γ (IgG), or μ (IgM), some of which may be further divided into subclasses, e.g., γ₁ (IgG₁), γ₂ (IgG₂), γ₃ (IgG₃), γ₄ (IgG₄), α₁ (IgA₁) and α₂ (IgA₂). The light chain of an immunoglobulin may be assigned to one of two types, called kappa (κ) and lambda (A), based on the amino acid sequence of its constant domain. An immunoglobulin essentially consists of two Fab molecules and an Fc domain, linked via the immunoglobulin hinge region.

The term “antigen binding domain” refers to the part of an antibody that comprises the area which specifically binds to and is complementary to part or all of an antigen. An antigen binding domain may be provided by, for example, one or more antibody variable domains (also called antibody variable regions). Preferably, an antigen binding domain comprises an antibody light chain variable region (VL) and an antibody heavy chain variable region (VH).

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6th ed., W.H. Freeman and Co., page 91 (2007). A single VH or VL domain may be sufficient to confer antigen binding specificity.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one embodiment, for the VL, the subgroup is subgroup kappa I as in Kabat et al., supra. In one embodiment, for the VH, the subgroup is subgroup III as in Kabat et al., supra.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term IgG “isotype” or “subclass” as used herein is meant any of the subclasses of immunoglobulins defined by the chemical and antigenic characteristics of their constant regions.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain (also referred to herein as a “cleaved variant heavy chain”). This may be the case where the final two C-terminal amino acids of the heavy chain are glycine (G446) and lysine (K447, EU numbering). Therefore, the C-terminal lysine (Lys447), or the C-terminal glycine (Gly446) and lysine (K447), of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, M D, 1991 (see also above). A “subunit” of an Fc domain as used herein refers to one of the two polypeptides forming the dimeric Fc domain, i.e., a polypeptide comprising C-terminal constant regions of an immunoglobulin heavy chain, capable of stable self-association. For example, a subunit of an IgG Fc domain comprises an IgG CH2 and an IgG CH3 constant domain.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein particularly includes separate modifications made to each of the two Fc domain subunits desired to associate (i.e., the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus, (hetero)dimerization occurs between a polypeptide comprising the first Fc domain subunit and a polypeptide comprising the second Fc domain subunit, which might be non-identical in the sense that further components fused to each of the subunits (e.g., antigen binding moieties) are not the same. In some embodiments the modification promoting association comprises an amino acid mutation in the Fc domain, specifically an amino acid substitution. In a particular embodiment, the modification promoting association comprises a separate amino acid mutation, specifically an amino acid substitution, in each of the two subunits of the Fc domain.

An “activating Fc receptor” is an Fc receptor that following engagement by an Fc region of an antibody elicits signaling events that stimulate the receptor-bearing cell to perform effector functions. Activating Fc receptors include FcγRIIIa (CD16a), FcγRI (CD64), FcγRIIa (CD32), and FcαRI (CD89).

The term “effector functions” when used in reference to antibodies refer to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g., B cell receptor), and B cell activation.

As used herein, the term “effector cells” refers to a population of lymphocytes that display effector moiety receptors, e.g., cytokine receptors, and/or Fc receptors on their surface through which they bind an effector moiety, e.g., a cytokine, and/or an Fc region of an antibody and contribute to the destruction of target cells, e.g., tumor cells. Effector cells may for example mediate cytotoxic or phagocytic effects. Effector cells include, but are not limited to, effector T cells such as CD8⁺ cytotoxic T cells, CD4⁺ helper T cells, γδ T cells, NK cells, lymphokine-activated killer (LAK) cells and macrophages/monocytes.

As used herein, the terms “engineer,” “engineered,” and “engineering,” are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches. “Engineering”, particularly with the prefix “glyco-”, as well as the term “glycosylation engineering,” includes metabolic engineering of the glycosylation machinery of a cell, including genetic manipulations of the oligosaccharide synthesis pathways to achieve altered glycosylation of glycoproteins expressed in cells. Furthermore, glycosylation engineering includes the effects of mutations and cell environment on glycosylation. In one embodiment, the glycosylation engineering is an alteration in glycosyltransferase activity. In a particular embodiment, the engineering results in altered glucosaminyltransferase activity and/or fucosyltransferase activity. Glycosylation engineering can be used to obtain a “host cell having increased GnTIII activity” (e.g., a host cell that has been manipulated to express increased levels of one or more polypeptides having β(1,4)-N-acetylglucosaminyltransferase III (GnTIII) activity), a “host cell having increased ManII activity” (e.g., a host cell that has been manipulated to express increased levels of one or more polypeptides having a-mannosidase II (ManII) activity), or a “host cell having decreased α(1,6) fucosyltransferase activity” (e.g., a host cell that has been manipulated to express decreased levels of α(1,6) fucosyltransferase).

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate proteins used for the present invention. In one embodiment, the host cell is engineered to allow the production of an antibody with modified oligosaccharides. In certain embodiments, the host cells have been manipulated to express increased levels of one or more polypeptides having β(1,4)-N-acetylglucosaminyltransferase III (GnTIII) activity. In certain embodiments the host cells have been further manipulated to express increased levels of one or more polypeptides having α-mannosidase II (ManII) activity. Host cells include cultured cells, e.g., mammalian cultured cells, such as CHO cells, BHK cells, NS0 cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

As used herein, the term “polypeptide having GnTIII activity” refers to a polypeptide that is able to catalyze the addition of a N-acetylglucosamine (GlcNAc) residue in β-1,4 linkage to the β-linked mannoside of the trimannosyl core of N-linked oligosaccharides. This includes fusion polypeptides exhibiting enzymatic activity similar to, but not necessarily identical to, an activity of β(1,4)-N-acetylglucosaminyltransferase III, also known as β-1,4-mannosyl-glycoprotein 4-beta-N-acetylglucosaminyl-transferase (EC 2.4.1.144), according to the Nomenclature Committee of the International Union of Biochemistry and Molecular Biology (NC-IUBMB), as measured in a particular biological assay, with or without dose dependency. In the case where dose dependency does exist, it need not be identical to that of GnTIII, but rather substantially similar to the dose-dependency in a given activity as compared to the GnTIII (i.e., the candidate polypeptide will exhibit greater activity or not more than about 25-fold less and, preferably, not more than about ten-fold less activity, and most preferably, not more than about three-fold less activity relative to the GnTIII). In certain embodiments the polypeptide having GnTIII activity is a fusion polypeptide comprising the catalytic domain of GnTIII and the Golgi localization domain of a heterologous Golgi resident polypeptide. Particularly, the Golgi localization domain is the localization domain of mannosidase II or GnTI, most particularly the localization domain of mannosidase II. Alternatively, the Golgi localization domain is selected from the group consisting of: the localization domain of mannosidase I, the localization domain of GnTII, and the localization domain of α1,6 core fucosyltransferase. Methods for generating such fusion polypeptides and using them to produce antibodies with increased effector functions are disclosed in WO2004/065540, U.S. Provisional Pat. Appl. No. 60/495,142 and U.S. Pat. Appl. Publ. No. 2004/0241817, the entire contents of which are expressly incorporated herein by reference.

As used herein, the term “Golgi localization domain” refers to the amino acid sequence of a Golgi resident polypeptide which is responsible for anchoring the polypeptide to a location within the Golgi complex. Generally, localization domains comprise amino terminal “tails” of an enzyme.

As used herein, the term “polypeptide having ManII activity” refers to polypeptides that are able to catalyze the hydrolysis of the terminal 1,3- and 1,6-linked α-D-mannose residues in the branched GlcNAcMan5GlcNAc2 mannose intermediate of N-linked oligosaccharides. This includes polypeptides exhibiting enzymatic activity similar to, but not necessarily identical to, an activity of Golgi a-mannosidase II, also known as mannosyl oligosaccharide 1,3-1,6-α-mannosidase II (EC 3.2.1.114), according to the Nomenclature Committee of the International Union of Biochemistry and Molecular Biology (NC-IUBMB).

Antibody-dependent cell-mediated cytotoxicity (ADCC) is an immune mechanism leading to the lysis of antibody-coated target cells by immune effector cells. The target cells are cells to which antibodies or fragments thereof comprising an Fc region specifically bind, generally via the protein part that is N-terminal to the Fc region. As used herein, the term “increased/reduced ADCC” is defined as either an increase/reduction in the number of target cells that are lysed in a given time, at a given concentration of antibody in the medium surrounding the target cells, by the mechanism of ADCC defined above, and/or a reduction/increase in the concentration of antibody, in the medium surrounding the target cells, required to achieve the lysis of a given number of target cells in a given time, by the mechanism of ADCC. The increase/reduction in ADCC is relative to the ADCC mediated by the same antibody produced by the same type of host cells, using the same standard production, purification, formulation and storage methods (which are known to those skilled in the art), but that has not been engineered. For example the increase in ADCC mediated by an antibody produced by host cells engineered to have an altered pattern of glycosylation (e.g., to express the glycosyltransferase, GnTIII, or other glycosyltransferases) by the methods described herein, is relative to the ADCC mediated by the same antibody produced by the same type of non-engineered host cells.

By “antibody having increased/reduced antibody dependent cell-mediated cytotoxicity (ADCC)” is meant an antibody having increased/reduced ADCC as determined by any suitable method known to those of ordinary skill in the art. One accepted in vitro ADCC assay is as follows:
1) the assay uses target cells that are known to express the target antigen recognized by the antigen-binding region of the antibody;
2) the assay uses human peripheral blood mononuclear cells (PBMCs), isolated from blood of a randomly chosen healthy donor, as effector cells;
3) the assay is carried out according to following protocol:
   i) the PBMCs are isolated using standard density centrifugation procedures and are suspended at 5×10 6 cells/mL in RPMI cell culture medium;
   ii) the target cells are grown by standard tissue culture methods, harvested from the exponential growth phase with a viability higher than 90%, washed in RPMI cell culture medium, labeled with 100 micro-Curies of 51 Cr, washed twice with cell culture medium, and resuspended in cell culture medium at a density of 10 5 cells/mL;
   iii) 100 microliters of the final target cell suspension above are transferred to each well of a 96-well microtiter plate;
   iv) the antibody is serially-diluted from 4000 ng/mL to 0.04 ng/mL in cell culture medium and 50 microliters of the resulting antibody solutions are added to the target cells in the 96-well microtiter plate, testing in triplicate various antibody concentrations covering the whole concentration range above;
   v) for the maximum release (MR) controls, 3 additional wells in the plate containing the labeled target cells, receive 50 microliters of a 2% (V/V) aqueous solution of non-ionic detergent (Nonidet, Sigma, St. Louis), instead of the antibody solution (point iv above);
   vi) for the spontaneous release (SR) controls, 3 additional wells in the plate containing the labeled target cells, receive 50 microliters of RPMI cell culture medium instead of the antibody solution (point iv above);
   vii) the 96-well microtiter plate is then centrifuged at 50×g for 1 minute and incubated for 1 hour at 4° C.;
   viii) 50 microliters of the PBMC suspension (point i above) are added to each well to yield an effector:target cell ratio of 25:1 and the plates are placed in an incubator under 5% CO₂ atmosphere at 37° C. for 4 hours;
   ix) the cell-free supernatant from each well is harvested and the experimentally released radioactivity (ER) is quantified using a gamma counter;
   x) the percentage of specific lysis is calculated for each antibody concentration according to the formula (ER-MR)/(MR-SR)×100, where ER is the average radioactivity quantified (see point ix above) for that antibody concentration, MR is the average radioactivity quantified (see point ix above) for the MR controls (see point v above), and SR is the average radioactivity quantified (see point ix above) for the SR controls (see point vi above);
4) “increased/reduced ADCC” is defined as either an increase/reduction in the maximum percentage of specific lysis observed within the antibody concentration range tested above, and/or a reduction/increase in the concentration of antibody required to achieve one half of the maximum percentage of specific lysis observed within the antibody concentration range tested above. The increase/reduction in ADCC is relative to the ADCC, measured with the above assay, mediated by the same antibody, produced by the same type of host cells, using the same standard production, purification, formulation and storage methods, which are known to those skilled in the art, but that has not been engineered.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures.

For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

As used herein, the terms “first,” “second,” “third,” etc. with respect to antigen binding moieties or domains, are used for convenience of distinguishing when there is more than one of each type of moiety or domain. Use of these terms is not intended to confer a specific order or orientation unless explicitly so stated.

The terms “multispecific” and “bispecific” mean that the antigen binding molecule is able to specifically bind to at least two distinct antigenic determinants. Typically, a bispecific antigen binding molecule comprises two antigen binding sites, each of which is specific for a different antigenic determinant. In certain embodiments, a bispecific antigen binding molecule is capable of simultaneously binding two antigenic determinants, particularly two antigenic determinants expressed on two distinct cells.

The term “valent” or “valency” as used herein denotes the presence of a specified number of antigen binding sites in an antigen binding molecule. As such, the term “monovalent binding to an antigen” denotes the presence of one (and not more than one) antigen binding site specific for the antigen in the antigen binding molecule.

An “antigen binding site” refers to the site, i.e., one or more amino acid residues, of an antigen binding molecule which provides interaction with the antigen. For example, the antigen binding site of an antibody comprises amino acid residues from the complementarity determining regions (CDRs). A native immunoglobulin molecule typically has two antigen binding sites, a Fab molecule typically has a single antigen binding site.

An “activating T cell antigen” as used herein refers to an antigenic determinant expressed by a T lymphocyte, particularly a cytotoxic T lymphocyte, which is capable of inducing or enhancing T cell activation upon interaction with an antigen binding molecule. Specifically, interaction of an antigen binding molecule with an activating T cell antigen may induce T cell activation by triggering the signaling cascade of the T cell receptor complex. An exemplary activating T cell antigen is CD3. In a particular embodiment the activating T cell antigen is CD3, particularly the epsilon subunit of CD3 (see UniProt no. P07766 (version 130), NCBI RefSeq no. NP_000724.1, for the human sequence; or UniProt no. Q95LI5 (version 49), NCBI GenBank no. BAB71849.1, for the cynomolgus [Macaca fascicularis] sequence).

“T cell activation” as used herein refers to one or more cellular response of a T lymphocyte, particularly a cytotoxic T lymphocyte, selected from: proliferation, differentiation, cytokine secretion, cytotoxic effector molecule release, cytotoxic activity, and expression of activation markers. The T cell activating therapeutic agents used in the present invention are capable of inducing T cell activation. Suitable assays to measure T cell activation are known in the art described herein.

A “target cell antigen” as used herein refers to an antigenic determinant presented on the surface of a target cell, for example a cell in a tumor such as a cancer cell or a cell of the tumor stroma. In a particular embodiment, the target cell antigen is CD20, particularly human CD20 (see UniProt no. P11836).

A “B-cell antigen” as used herein refers to an antigenic determinant presented on the surface of a B lymphocyte, particularly a malignant B lymphocyte (in that case the antigen also being referred to as “malignant B-cell antigen”).

A “T-cell antigen” as used herein refers to an antigenic determinant presented on the surface of a T lymphocyte, particularly a cytotoxic T lymphocyte.

A “Fab molecule” refers to a protein consisting of the VH and CH1 domain of the heavy chain (the “Fab heavy chain”) and the VL and CL domain of the light chain (the “Fab light chain”) of an immunoglobulin.

By “fused” is meant that the components (e.g., a Fab molecule and an Fc domain subunit) are linked by peptide bonds, either directly or via one or more peptide linkers.

An “effective amount” of an agent refers to the amount that is necessary to result in a physiological change in the cell or tissue to which it is administered.

A “therapeutically effective amount” of an agent, e.g., a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

By “therapeutic agent” is meant an active ingredient, e.g., of a pharmaceutical composition, that is administered to a subject in an attempt to alter the natural course of a disease in the subject being treated, and can be performed either for prophylaxis or during the course of clinical pathology. An “immunotherapeutic agent” refers to a therapeutic agent that is administered to a subject in an attempt to restore or enhance the subject's immune response, e.g., to a tumor.

The term “pharmaceutical composition” refers to a preparation which is in such form as to permit the biological activity of an active ingredient contained therein to be effective, and which contains no additional components which are unacceptably toxic to a subject to which the composition would be administered.

A “pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative.

The term “package insert” or “instructions for use” is used to refer to instructions customarily included in commercial packages of therapeutic products that contain information about the indications, usage, dosage, administration, combination therapy, contraindications and/or warnings concerning the use of such therapeutic products.

The term “combination treatment” noted herein encompasses combined administration (where two or more therapeutic agents are included in the same or separate formulations), and separate administration, in which case, administration of an antibody as reported herein can occur prior to, simultaneously, and/or following, administration of the additional therapeutic agent or agents, preferably an antibody or antibodies.

By a “crossover” Fab molecule (also termed “Crossfab”) is meant a Fab molecule wherein the variable domains or the constant domains of the Fab heavy and light chain are exchanged (i.e., replaced by each other), i.e., the crossover Fab molecule comprises a peptide chain composed of the light chain variable domain VL and the heavy chain constant domain 1 CH1 (VL-CH1, in N- to C-terminal direction), and a peptide chain composed of the heavy chain variable domain VH and the light chain constant domain CL (VH-CL, in N- to C-terminal direction). For clarity, in a crossover Fab molecule wherein the variable domains of the Fab light chain and the Fab heavy chain are exchanged, the peptide chain comprising the heavy chain constant domain 1 CH1 is referred to herein as the “heavy chain” of the (crossover) Fab molecule. Conversely, in a crossover Fab molecule wherein the constant domains of the Fab light chain and the Fab heavy chain are exchanged, the peptide chain comprising the heavy chain variable domain VH is referred to herein as the “heavy chain” of the (crossover) Fab molecule.

In contrast thereto, by a “conventional” Fab molecule is meant a Fab molecule in its natural format, i.e., comprising a heavy chain composed of the heavy chain variable and constant domains (VH-CH1, in N- to C-terminal direction), and a light chain composed of the light chain variable and constant domains (VL-CL, in N- to C-terminal direction).

The term “polynucleotide” refers to an isolated nucleic acid molecule or construct, e.g., messenger RNA (mRNA), virally-derived RNA, or plasmid DNA (pDNA). A polynucleotide may comprise a conventional phosphodiester bond or a non-conventional bond (e.g., an amide bond, such as found in peptide nucleic acids (PNA). The term “nucleic acid molecule” refers to any one or more nucleic acid segments, e.g., DNA or RNA fragments, present in a polynucleotide.

By “isolated” nucleic acid molecule or polynucleotide is intended a nucleic acid molecule, DNA or RNA, which has been removed from its native environment. For example, a recombinant polynucleotide encoding a polypeptide contained in a vector is considered isolated for the purposes of the present invention. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts of the present invention, as well as positive and negative strand forms, and double-stranded forms. Isolated polynucleotides or nucleic acids according to the present invention further include such molecules produced synthetically. In addition, a polynucleotide or a nucleic acid may be or may include a regulatory element such as a promoter, ribosome binding site, or a transcription terminator.

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98%, or 99% identical to a nucleotide sequence of the present invention can be determined conventionally using known computer programs, such as the ones discussed above for polypeptides (e.g., ALIGN-2).

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette of the invention comprises polynucleotide sequences that encode bispecific antigen binding molecules of the invention or fragments thereof.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector of the invention comprises an expression cassette that comprises polynucleotide sequences that encode bispecific antigen binding molecules of the invention or fragments thereof.

The term “about” as used herein refers to the usual error range for the respective value readily known to the skilled person in this technical field. Reference to “about” a value or parameter herein includes (and describes) embodiments that are directed to that value or parameter per se.

By “B cell proliferative disorder” is meant a disease wherein the number of B cells in a patient is increased as compared to the number of B cells in a healthy subject, and particularly wherein the increase in the number of B cells is the cause or hallmark of the disease. A “CD20-positive B cell proliferative disorder” is a B cell proliferative disorder wherein B-cells, particularly malignant B-cells (in addition to normal B-cells), express CD20.

Exemplary B cell proliferation disorders include Non-Hodgkin lymphoma (NHL), diffuse large B-cell lymphoma (DLBCL; e.g., relapsed or refractory DLBCL not otherwise specified (NOS), high grade B cell lymphoma (HGBCL; e.g., HGBCL NOS, double-hit HGBCL, and triple-hit HGBCL), primary mediastinal large B-cell lymphoma (PMBCL), and DLBCL arising from FL (transformed FL; trFL)); follicular lymphoma (FL), including Grades 1-3b FL; mantle-cell lymphoma (MCL); and marginal zone lymphoma (MZL), including splenic, nodal or extra-nodal MZL. In one embodiment, the CD20-positive B cell proliferative disorder is a Burkitt lymphoma (BL); a Burkitt leukemia (BAL; mature B-cell leukemia FAB L3); DLBCL, or PMBCL. In one embodiment the CD20-positive B cell proliferative disorder is a relapsed or refractory NHL (e.g., a relapsed or refractory DLBCL, a relapsed or refractory FL, or a relapsed or refractory MCL). In one embodiment, the BL, BAL, DLBCL, or PMBCL is relapsed and/or refractory. In one embodiment, the BL, BAL, DLBCL, or PMBCL has relapsed after or is refractory to a first-line standard-of-care chemoimmunotherapy.

“Refractory disease” is defined as failure to achieve complete remission to first-line therapy, including defined as failure to achieve complete remission to first-line therapy, including:
Progressive Disease (PD) as best response to first-line therapy
Stable disease (SD) as best response after at least 4 cycles of first-line therapy (e.g., 4 cycles of R-CHOP)
Partial Response (PR) as best response after at least 6 cycles and with either biopsy-proven residual disease or subsequent disease progression.

“Relapsed disease” is defined as complete remission to first-line therapy. In one embodiment disease relapse is proven by biopsy. In one embodiment, patients have relapsed after or failed to respond to at least two prior systemic treatment regimens (including at least one prior regimen containing anthracycline, and at least one containing an anti CD20-directed therapy).

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). Preferably, the individual or subject is a human. In one instance, each subject in a population of subjects is human. In one instance, each subject in a reference population of subjects is human. In one embodiment, a subject is considered to be a pediatric patient if the pediatric patient is younger than 18 years old (i.e., aged 17 years or less). In one embodiment, the pediatric patient is aged between 6 months and 17 years. In one embodiment, a subject is considered to be a young adult patient if the young adult patient is aged between 18 years and 30 years.

A “transplant eligible” subject or a subject “eligible for autologous stem cell transplantation (SCT)” is a subject who meets eligibility for, who is recommended for or who can receive, autologous SCT. In one embodiment, “transplant eligible” is defined as being medically eligible for intensive platinum-based salvage therapy followed by autologous stem cell transplantation (ASCT). In one embodiment the transplant eligible subject achieves an objective response as well as mobilization of the target dose of at least 2,000,000 CD34+ hematopoietic stem cells/kg. In one embodiment, “transplant eligible” is defined as being medically eligible for two to three cycles of salvage therapy with R-ICE and glofitamab to achieve CR followed by allogeneic or autologous hematopoietic stem cell transplantation (HSCT).

A “CAR-T cell therapy eligible” subject or a subject “eligible for CAR-T cell therapy” is a subject who meets eligibility for, who is recommended for or who can receive, chimeric antigen receptor (CAR) T-cell therapy.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, methods of the invention are used to delay development of a disease or to slow the progression of a disease. In one embodiment, the disease being treated is a CD20-positive B cell proliferative disorder, e.g., a Burkitt lymphoma (BL); a Burkitt leukemia (BAL; mature B-cell leukemia FAB L3); DLBCL, or PMBCL.

As used herein, “delaying progression” of a disorder or disease means to defer, hinder, slow, retard, stabilize, and/or postpone development of the disease or disorder (e.g., a CD20-positive B cell proliferative disorder, e.g., NHL, e.g., DLBCL, e.g., Burkitt lymphoma (BL); e.g., Burkitt leukemia (BAL; mature B-cell leukemia FAB L3), or e.g., PMBCL). This delay can be of varying length of time, depending on the history of the disease and/or individual being treated. As is evident to one skilled in the art, a sufficient or significant delay can, in effect, encompass prevention, in that the individual does not develop the disease. For example, in a late-stage cancer, development of central nervous system (CNS) metastasis, may be delayed.

By “reduce” or “inhibit” is meant the ability to cause an overall decrease, for example, of 20%, 30%, 40%, 50%, 60%, 70%, 75%, 80%, 85%, 90%, 95%, or greater. For clarity the term includes also reduction to zero (or below the detection limit of the analytical method), i.e., complete abolishment or elimination. In certain embodiments, reduce or inhibit can refer to the reduction or inhibition of undesirable events, such as cytokine-driven toxicities (e.g., cytokine release syndrome (CRS)), infusion-related reactions (IRRs), macrophage activation syndrome (MAS), neurologic toxicities, severe tumor lysis syndrome (TLS), neutropenia, thrombocytopenia, elevated liver enzymes, and/or central nervous system (CNS) toxicities, following treatment with an anti-CD20/anti-CD3 bispecific antibody using the step-up dosing regimen of the invention relative to unchanging, preset dosing with the target dose of the bispecific antibody. In other embodiments, reduce or inhibit can refer to effector function of an antibody that is mediated by the antibody Fc region, such effector functions specifically including complement-dependent cytotoxicity (CDC), antibody-dependent cellular cytotoxicity (ADCC), and antibody-dependent cellular phagocytosis (ADCP). In other embodiments reduce or inhibit can refer to the symptoms of the CD20-positive B cell proliferative disorder being treated (e.g., an NHL (e.g., a DLBCL), an FL (e.g., a relapsed and/or refractor FL or a transformed FL), an MCL, a high-grade B cell lymphoma, or a PMLBCL), the presence or size of metastases, or the size of the primary tumor. In one embodiment, the CD20-positive B cell proliferative disorder being treated is a Burkitt lymphoma (BL); a Burkitt leukemia (BAL; mature B-cell leukemia FAB L3); DLBCL, or PMBCL.

As used herein, “administering” is meant a method of giving a dosage of a compound (e.g., an anti-CD20/anti-CD3 bispecific antibody) or a composition (e.g., a pharmaceutical composition, e.g., a pharmaceutical composition including an anti-CD20/anti-CD3 bispecific antibody) to a subject. The compounds and/or compositions utilized in the methods described herein can be administered intravenously (e.g., by intravenous infusion).

A “fixed” or “flat” dose of a therapeutic agent (e.g., a bispecific antibody) herein refers to a dose that is administered to a patient without regard for the weight or body surface area (BSA) of the patient.

The fixed or flat dose is therefore not provided as a mg/kg dose or a mg/m² dose, but rather as an absolute amount of the therapeutic agent (e.g., mg).

A “target dose” herein refers to the dose of the anti-CD20/anti-CD3 bispecific antibody that achieves therapeutic effect, i.e., achieves the desired clinical efficacy. It was found that for glofitamab a possible target dose is 16 mg or 30 mg. In one embodiment, the target dose for a pediatric subject (i.e., aged 6 months to 17 years) is 0.5 mg/kg or 0.4 mg/kg depending on the subject's weight. In a particular embodiment, the target dose is 0.5 mg/kg for a pediatric subject weighing 7.5 to <13 kg, 0.4 mg/kg for a pediatric subject weighting ≥13 kg to <45 kg, and 30 mg flat dose for a pediatric subject weighing ≥45 kg.

An “unchanging or preset dosing with target dose” and a “treatment regimen without a step-up dosing regimen” refers to a dosing schedule that uses the same dosage in the first and second cycle and optionally also any subsequent treatment cycle, as opposed to step-up dosing, which uses lower dosages in the first few treatment cycles and only reaches the target dose in the second or in a later treatment cycle.

The terms “treatment cycle” or “cycle” (abbreviated: “C”) as used herein mean a course of one or more doses of the anti-CD20/anti-CD3 bispecific antibody that is repeated on a regular schedule, optionally with periods of rest (no treatment) in between. In one aspect of the invention, the first treatment cycle comprises a first and a second dose of the anti-CD20/anti-CD3 bispecific antibody, followed by a period of rest. In one such embodiment, the first treatment cycle comprises a first dose of the anti-CD20/anti-CD3 bispecific antibody on Day 8 of the first cycle, and a second dose of the anti-CD20/anti-CD3 bispecific antibody on Day 15 of the first cycle, followed by 6 days of rest. In one embodiment the second and any subsequent cycles comprise one dose of the anti-CD20/anti-CD3 bispecific antibody given at Day 8 of that cycle, followed by 13 days of rest. In one embodiment, one treatment cycle comprises 21 days. In another embodiment, one treatment cycle comprises 14 days. The treatment schedule according to the invention may comprise 2 or more treatment cycles, in particular 3 treatment cycles. In some embodiments, a treatment cycle is referred to as a “dosing cycle.”

“Individual response” or “response” can be assessed using any endpoint indicating a benefit to the subject, including, without limitation, (1) inhibition, to some extent, of disease progression (e.g., progression of a CD20-positive B cell proliferative disorder, e. g., a non-Hodgkin's lymphoma (NHL)); including slowing down and complete arrest; (2) a reduction in tumor size; (3) inhibition (i.e., reduction, slowing down or complete stopping) of cancer cell infiltration into adjacent peripheral organs and/or tissues; (4) inhibition (i.e., reduction, slowing down or complete stopping) of metastasis; (5) relief, to some extent, of one or more symptoms associated with the CD20-positive B cell proliferative disorder, e.g., a B cell proliferative disorder; (6) increase or extend in the length of survival, including overall survival and progression-free survival; and/or (7) decreased mortality at a given point of time following treatment.

As used herein, “complete response” or “CR” refers to disappearance of all target lesions. In one embodiment standard NHL response criteria are assessed for determining CR. (Lugano Classification, Cheson et al. J Clin Oncol. 2014 Sep. 20; 32(27): 3059-3067.). In one embodiment the CR rate is defined as the proportion of participants that achieves a CR within three cycles of glofit-R-ICE, as determined by the investigator according to Lugano criteria. In one embodiment CR is defined as complete metabolic response as determined by PET/CT of the lymph nodes and extra-lymphatic sites, with a score of 1, 2, or 3 with or without a residual mass on 5PS, wherein PET 5PS: 1=no uptake above background; 2=uptake>mediastinum; 3=uptake>mediastinum but ≤liver; 4=uptake moderately>liver; 5=uptake markedly higher than liver and/or new lesions; X=new areas of uptake unlikely to be related to lymphoma. In one embodiment, CR is defined as complete radiologic response as determined by CT of the lymph nodes and extra-lymphatic sites, wherein the Target nodes/nodal masses must regress to ≤1.5 cm in LDi (longest transverse diameter of a lesion) and no extralymphatic sites of disease remain. For pediatric subjects (<18 years old), CR is assessed using the International Pediatric NHL Response Criteria (Sandlund J T, Guillerman R P, Perkins S L, et al. International pediatric non-Hodgkin lymphoma response criteria. J. Clin. Oncol. 33:2106-2111, 2015).

As used herein, “partial response” or “PR” refers to Partial metabolic response as determined by PET/CT of the lymph nodes and extra-lymphatic sites and/or Partial remission as determined by CT of the lymph nodes and extra-lymphatic sites. In one embodiment, Partial metabolic response is defined by a score 4 or 5 b with reduced uptake compared with baseline and residual mass(es) of any size as determined by PET/CT of the lymph nodes and extra-lymphatic sites, wherein PET 5PS: 1=no uptake above background; 2=uptake>mediastinum; 3=uptake>mediastinum but liver; 4=uptake moderately>liver; 5=uptake markedly higher than liver and/or new lesions; X=new areas of uptake unlikely to be related to lymphoma. In one embodiment partial remission is defined as at least a 50% decrease in the product of the diameters (SPD) of up to 6 target measurable nodes and extranodal sites, taking as reference the baseline SPD. For pediatric subjects (<18 years old), PR is assessed using the International Pediatric NHL Response Criteria (Sandlund J T, Guillerman R P, Perkins S L, et al. International pediatric non-Hodgkin lymphoma response criteria. J. Clin. Oncol. 33:2106-2111, 2015).

An “effective response” of a subject or a subject's “responsiveness” to treatment with a medicament and similar wording refers to the clinical or therapeutic benefit imparted to a subject as risk for, or suffering from, a disease or disorder, such as cancer. In one embodiment, such benefit includes any one or more of: extending survival (including overall survival and progression free survival); resulting in an objective response (including a complete response or a partial response); or improving signs or symptoms of cancer.

“Duration of complete response” (DOCR) is defined as the time from the first occurrence of a documented complete response to disease progression or death from any cause (whichever occurs first), as determined by the investigator according to Lugano criteria (Cheson et al. J Clin Oncol. 2014 Sep. 20; 32(27): 3059-3067.). CR is assessed by the investigator, using the International Pediatric NHL Response Criteria for pediatric subjects <18 years old (Sandlund J T, Guillerman R P, Perkins S L, et al. International pediatric non-Hodgkin lymphoma response criteria. J. Clin. Oncol. 33:2106-2111, 2015).

“Duration of objective response” (DOR), defined as the time from the first occurrence of a documented objective response (CR or PR) to disease progression or death from any cause (whichever occurs first), as determined by the investigator according to Lugano criteria (Cheson et al. J Clin Oncol. 2014 Sep. 20; 32(27): 3059-3067.). CR and/or PR is assessed by the investigator, using the International Pediatric NHL Response Criteria for pediatric subjects <18 years old (Sandlund J T, Guillerman R P, Perkins S L, et al. International pediatric non-Hodgkin lymphoma response criteria. J. Clin. Oncol. 33:2106-2111, 2015).

“Progression-free survival” (PFS) is defined as the time from the first treatment with the anti-CD20/anti-CD3 bispecific antibody to the first occurrence of disease progression or death from any cause, whichever occurs first. In one embodiment, PFS is assessed based on the Lugano Classification (Cheson et al. J Clin Oncol. 2014 Sep. 20; 32(27): 3059-3067.). PFS is assessed by the investigator, using the International Pediatric NHL Response Criteria for pediatric subjects <18 years old (Sandlund J T, Guillerman R P, Perkins S L, et al. International pediatric non-Hodgkin lymphoma response criteria. J. Clin. Oncol. 33:2106-2111, 2015).

“Overall survival” (OS) is defined as time from the first treatment with the anti-CD20/anti-CD3 bispecific antibody to the date of death from any cause.

As used herein “Event-free survival” (EFS) is defined as the time from the first treatment with the anti-CD20/anti-CD3 bispecific antibody the first occurrence of disease progression as determined by the investigator according to Lugano criteria, initiation of new anti-lymphoma therapy (not including planned ASCT), or death from any cause (whichever occurs first). EFS is assessed by the investigator, using the International Pediatric NHL Response Criteria for pediatric subjects <18 years old (Sandlund J T, Guillerman R P, Perkins S L, et al. International pediatric non-Hodgkin lymphoma response criteria. J. Clin. Oncol. 33:2106-2111, 2015).

As used herein, “objective response rate” (ORR) is defined as the sum of partial response (PR) rate and complete response (CR) rate. In one embodiment, ORR is evaluated based on the Lugano Classification (Cheson et al. J Clin Oncol. 2014 Sep. 20; 32(27): 3059-3067). In one embodiment, the ORR is defined as the proportion of participants that achieves a CR or PR within three cycles of the anti-CD20/anti-CD3 bispecific antibody (e.g., glofitamab)+R-ICE treatment regimen described therein. CR and/or PR is assessed by the investigator, using the International Pediatric NHL Response Criteria for pediatric subjects <18 years old (Sandlund J T, Guillerman R P, Perkins S L, et al. International pediatric non-Hodgkin lymphoma response criteria. J. Clin. Oncol. 33:2106-2111, 2015).

As used herein, “stable disease” or “SD” refers to neither sufficient shrinkage of target lesions to qualify for PR, nor sufficient increase to qualify for PD, taking as reference the smallest SLD since the treatment started.

As used herein, “progressive disease” or “PD” refers to at least a 20% increase in the SLD of target lesions, taking as reference the smallest SLD, or at least a 50% increase in the SPD of target legions, taking as reference the smallest SPD, recorded since the treatment started or the presence of one or more new lesions.

As used herein, an “infusion-related reaction,” “IRR,” or infusion-related adverse event” is an adverse event that occurs in a patient or subject during or within 24 hours after administration of a drug (e.g., an anti-CD20/anti-CD3 bispecific antibody, e.g., glofitamab; or an anti-CD20 antibody, e.g., obinutuzumab or rituximab). IRRs may be graded as Grades 1-5 according to, e.g., NCI CTCAE v.4.

“Mobilization-adjusted response rate (MARR)” is defined as the percentage of patients who achieve an objective response as well as mobilization of the target dose of 2,000,000 CD34+ hematopoietic stem cells/kg typically required as a minimum for ASCT.

As used herein, the term “R-ICE” refers to Rituximab plus ifosfamide, carboplatin, etoposide or etoposide phosphate. As used herein, the term “ICE,” refers to ifosfamide, carboplatin, etoposide or etoposide phosphate.

(iii) Combination Treatment of an Anti-CD20/Anti-CD3 Bispecific Antibody with an Anti-CD20 Antibody and Chemotherapy

The invention provides methods for treating a subject having a CD20-positive cell proliferative disorder (e.g., a B cell proliferative disorder (e.g., non-Hodgkin's lymphoma (NHL) (e.g., a relapsed and/or refractory NHL, a diffuse-large B cell lymphoma (DLBCL) (e.g., a relapsed and/or refractory DLBCL), a follicular lymphoma (FL) (e.g., a relapsed and/or refractory FL or a transformed FL), or a mantle cell lymphoma (MCL) (e.g., a relapsed or refractory MCL)), or a central nervous system lymphoma (CNSL))) that includes administering to the subject an anti-CD20/anti-CD3 bispecific antibody in combination with an anti-CD20 antibody and one or more chemotherapeutic agents selected from ifosfamide, carboplatin and/or etoposide.

In some instances, the present methods are used for treating a subject having relapsed and/or refractory NHL (e.g., an aggressive NHL (e.g., a relapsed and/or refractory DLBCL, a relapsed and/or refractory FL, or a relapsed and/or refractory MCL)). In some instances, the subject has relapsed following one or more (e.g., one, two, three, four, five, or more) prior therapies (e.g., one or more prior systemic therapies, e.g., one or more prior systemic chemotherapies (e.g., one or more prior systemic therapies involving administration of anthracycline), one or more prior stem cell therapies, or one or more prior CAR-T cell therapies) after having a documented history of response (e.g., a complete response or a partial response) of at least 6 months in duration from completion of the therapy. In some instances, the subject is refractory to any prior therapy (e.g., has had no response to the prior therapy, or progression within 6 months of completion of the last dose of therapy). Thus, in some embodiments, the present dosing regimen is a second-line therapy. In some embodiments, the present dosing regimen is a third-line therapy. In some embodiments, the subject has a transformed FL, which is a refractory to standard therapies for transformed FL. In some embodiments, the FL is a Graded FL (e.g., a Grade 1, 2, 3a, or 3b FL).

In one aspect, the invention features a method of treating a subject having a CD20-positive cell proliferative disorder, e.g., a B cell proliferative disorder (e.g., an NHL (e.g., a relapsed and/or refractory NHL, a DLBCL (e.g., a relapsed and/or refractory DLBCL), a FL (e.g., a relapsed and/or refractory FL or a transformed FL), or an MCL (e.g., a relapsed or refractory MCL)), or a CNSL), comprising administering to the subject an effective amount of:
(a) a bispecific antibody that binds to CD20 and CD3;
(b) an anti-CD20 antibody; and
(c) one or more chemotherapeutic agents selected from ifosfamide, carboplatin and/or etoposide in a dosing regimen comprising at least a first dosing cycle and a second dosing cycle.

In one embodiment, the first dosing cycle comprises a first dose (C1D1) of the bispecific antibody and a second dose (C1D2) of the bispecific antibody, wherein the C1D1 of the bispecific antibody is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of the bispecific antibody is about 10 mg (e.g., 10 mg±0.05 mg, ±0.1 mg, ±0.2 mg, ±0.3 mg, ±0.5 mg, ±0.75 mg, or ±1 mg); and the second dosing cycle comprises a single dose (C2D1) of the bispecific antibody, wherein the C2D1 of the bispecific antibody is about 10 mg (e.g., 10 mg±0.05 mg, ±0.1 mg, ±0.2 mg, ±0.3 mg, ±0.5 mg, ±0.75 mg, or ±1 mg), about 16 mg (e.g., 16 mg±0.05 mg, ±0.1 mg, ±0.2 mg, ±0.3 mg, ±0.5 mg, ±0.75 mg, ±1 mg, ±1.5 mg, or ±1.6 mg), or about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg). In particular embodiments, the C1D1 of the bispecific antibody is about 2.5 mg (e.g., 2.5 mg±0.01 mg, ±0.02 mg, ±0.03 mg, ±0.05 mg, ±0.1 mg, ±0.2 mg, or ±0.25 mg) and the C1D2 of the bispecific antibody is about 10 mg (e.g., 10 mg±0.05 mg, ±0.1 mg, ±0.2 mg, ±0.3 mg, ±0.5 mg, ±0.75 mg, or ±1 mg). In particular embodiments, the C2D1 is about 10 mg (e.g., 10 mg±0.05 mg, ±0.1 mg, ±0.2 mg, ±0.3 mg, ±0.5 mg, ±0.75 mg, or ±1 mg). In particular embodiments, the C2D1 of the bispecific antibody is about 16 mg (e.g., 16 mg±0.05 mg, ±0.1 mg, ±0.2 mg, ±0.3 mg, ±0.5 mg, ±0.75 mg, ±1 mg, ±1.5 mg, or ±1.6 mg). In particular embodiments, the C2D1 of the bispecific antibody is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one embodiment, the first dosing cycle comprises a first dose (C1D1) of the bispecific antibody and a second dose (C1D2) of the bispecific antibody, wherein the C1D1 of the bispecific antibody is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of the bispecific antibody is about 10 mg (e.g., 10 mg±0.05 mg, ±0.1 mg, ±0.2 mg, ±0.3 mg, ±0.5 mg, ±0.75 mg, or ±1 mg); and the second dosing cycle comprises a single dose (C2D1) of the bispecific antibody, wherein the C2D1 of the bispecific antibody is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one embodiment, the first dose (C1D1) of the bispecific antibody and the second dose (C1D2) of the bispecific antibody are administered to the subject on or about Days 8 (±1 day) and 15 (±1 day), respectively, of the first dosing cycle.

In one embodiment, the first dose (C1D1) of the bispecific antibody and the second dose (C1D2) of the bispecific antibody are administered to the subject on Days 8 and 15, respectively, of the first dosing cycle. In some embodiments, the C2D1 of the bispecific antibody is administered to the subject on or about Day 8 (±1 day) of the second dosing cycle. In some embodiments, the C2D1 of the bispecific antibody is administered to the subject on or about Day 8 of the second dosing cycle.

In one embodiment, the anti-CD20 antibody is obinutuzumab and/or rituximab. In one embodiment, the first dosing cycle comprises a single dose (C1D1) of obinutuzumab; and the second dosing cycle comprises a single dose (C2D1) of rituximab.

In one embodiment, the single dose C1D1 of the obinutuzumab is about 1000 mg (e.g., 1000 mg ±5 mg, ±10 mg, ±20 mg, ±30 mg, ±50 mg, ±75 mg, or ±100 mg) and the single dose of rituximab is about 375 mg/m² (e.g., 375 mg/m²±5 mg/m², ±10 mg/m², ±25 mg/m², or ±37.5 mg/m²).

In one embodiment, the anti-CD20 antibody is administered in a dosing regimen comprising at least a first dosing cycle and a second dosing cycle, wherein the first dosing cycle comprises a single dose (C1D1) of obinutuzumab on Day 1; and the second dosing cycle comprises a single dose (C2D1) of rituximab on Day 1.

In one aspect, the invention features a method of treating a subject having a CD20-positive cell proliferative disorder, e.g., a B cell proliferative disorder (e.g., an NHL (e.g., a relapsed and/or refractory NHL, a DLBCL (e.g., a relapsed and/or refractory DLBCL), a FL (e.g., a relapsed and/or refractory FL or a transformed FL), or an MCL (e.g., a relapsed or refractory MCL)), or a CNSL), comprising administering to the subject in a first dosing cycle:
(a) about 2.5 mg (e.g., 2.5 mg±0.01 mg, ±0.02 mg, ±0.03 mg, ±0.05 mg, ±0.1 mg, ±0.2 mg, or ±0.25 mg) as the first dose (C1D1) of a bispecific antibody that binds to CD20 and CD3 and about 10 mg (e.g., 10 mg±0.05 mg, ±0.1 mg, ±0.2 mg, ±0.3 mg, ±0.5 mg, ±0.75 mg, or ±1 mg) as the second dose (C1D2) of the bispecific antibody that binds to CD20 and CD3;
(b) a single dose (C1D1) of obinutuzumab; and
(c) one or more chemotherapeutic agents selected from ifosfamide, carboplatin and/or etoposide;
and administering to the subject in a second dosing cycle:
(a) a single dose (C2D1) of about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg) of a bispecific antibody that binds to CD20 and CD3;
(b) a single dose (C2D1) of rituximab; and
(c) one or more chemotherapeutic agents selected from ifosfamide, carboplatin and/or etoposide.

In one embodiment, the single dose C1D1 of the obinutuzumab is about 1000 mg (e.g., 1000 mg ±5 mg, ±10 mg, ±20 mg, ±30 mg, ±50 mg, ±75 mg, or ±100 mg) and the single dose of rituximab is about 375 mg/m² (e.g., 375 mg/m²±5 mg/m², ±10 mg/m², ±25 mg/m², or ±37.5 mg/m²).

In one embodiment, the anti-CD20 antibody is administered in a dosing regimen comprising at least a first dosing cycle and a second dosing cycle, wherein the first dosing cycle comprises a single dose (C1D1) of obinutuzumab on Day 1; and the second dosing cycle comprises a single dose (C2D1) of rituximab on Day 1.

In one embodiment, the first dose (C1D1) of the bispecific antibody and the second dose (C1D2) of the bispecific antibody are administered to the subject on or about Days 8 (±1 day) and 15 (±1 day), respectively, of the first dosing cycle.

In one embodiment, the first dose (C1D1) of the bispecific antibody and the second dose (C1D2) of the bispecific antibody are administered to the subject on Days 8 and 15, respectively, of the first dosing cycle. In some embodiments, the C2D1 of the bispecific antibody is administered to the subject on or about Day 8 (±1 day) of the second dosing cycle. In some embodiments, the C2D1 of the bispecific antibody is administered to the subject on or about Day 8 of the second dosing cycle.

In one embodiment of the methods provided above, step c) comprises all three chemotherapeutic agents.

In one embodiment, the first dosing cycle comprises a single dose (C1D1) of ifosfamide, a single dose (C1D1) of carboplatin and a first (C1D1), second (C1D2) and third (C1D3) dose of etoposide; and the second cycle each comprises a single dose (C2D1) of ifosfamide, a single dose (C2D1) of carboplatin and a first (C2D1), second (C2D2) and third (C2D3) dose of etoposide.

In one aspect, the invention features a method of treating a subject having a CD20-positive cell proliferative disorder, e.g., a B cell proliferative disorder (e.g., an NHL (e.g., a relapsed and/or refractory NHL, a DLBCL (e.g., a relapsed and/or refractory DLBCL), a FL (e.g., a relapsed and/or refractory FL or a transformed FL), or an MCL (e.g., a relapsed or refractory MCL)), or a CNSL), comprising administering to the subject in a first dosing cycle:
(a) about 2.5 mg (e.g., 2.5 mg±0.01 mg, ±0.02 mg, ±0.03 mg, ±0.05 mg, ±0.1 mg, ±0.2 mg, or ±0.25 mg) as the first dose (C1D1) of a bispecific antibody that binds to CD20 and CD3 and about 10 mg (e.g., 10 mg±0.05 mg, ±0.1 mg, ±0.2 mg, ±0.3 mg, ±0.5 mg, ±0.75 mg, or ±1 mg) as the second dose (C1D2) of the bispecific antibody that binds to CD20 and CD3;
(b) a single dose (C1D1) of obinutuzumab; and
(c) a single dose (C1D1) of ifosfamide, a single dose (C1D1) of carboplatin and a first (C1D1), second (C1D2) and third (C1D3) dose of etoposide;
and administering to the subject in a second dosing cycle:
(a) a single dose (C2D1) of about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg) of a bispecific antibody that binds to CD20 and CD3;
(b) a single dose (C2D1) of rituximab; and
(c) a single dose (C2D1) of ifosfamide, a single dose (C2D1) of carboplatin and a first (C2D1), second (C2D2) and third (C2D3) dose of etoposide.

In one embodiment, the single dose C1D1 of the obinutuzumab is about 1000 mg (e.g., 1000 mg ±5 mg, ±10 mg, ±20 mg, ±30 mg, ±50 mg, ±75 mg, or ±100 mg) and the single dose of rituximab is about 375 mg/m² (e.g., 375 mg/m²±5 mg/m², ±10 mg/m², ±25 mg/m², or ±37.5 mg/m²).

In one embodiment, the anti-CD20 antibody is administered in a dosing regimen comprising at least a first dosing cycle and a second dosing cycle, wherein the first dosing cycle comprises a single dose (C1D1) of obinutuzumab on Day 1; and the second dosing cycle comprises a single dose (C2D1) of rituximab on Day 1.

In one embodiment, the first dose (C1D1) of the bispecific antibody and the second dose (C1D2) of the bispecific antibody are administered to the subject on or about Days 8 (±1 day) and 15 (±1 day), respectively, of the first dosing cycle.

In one embodiment, the first dose (C1D1) of the bispecific antibody and the second dose (C1D2) of the bispecific antibody are administered to the subject on Days 8 and 15, respectively, of the first dosing cycle. In some embodiments, the C2D1 of the bispecific antibody is administered to the subject on or about Day 8 (±1 day) of the second dosing cycle. In some embodiments, the C2D1 of the bispecific antibody is administered to the subject on or about Day 8 of the second dosing cycle.

In one embodiment of the methods provided above ifosfamide is administered at a dose of 5000 mg/m², 4000 mg/m² or 1666 mg/m². In one embodiment of the methods provided above ifosfamide is administered at a dose of 5000 mg/m². In one embodiment of the methods provided above, carboplatin is administered at a dose in mg to target area under the curve (AUC) of 5 mg/mL/min with maximum dose of 750 mg. In one embodiment of the methods provided above etoposide is administered at a dose of 100 mg/m² or 75 mg/m². In one embodiment of the methods provided above etoposide is administered at a dose of 100 mg/m².

In one embodiment of the methods provided above ifosfamide is administered at a dose of 5000 mg/m², 4000 mg/m² or 1666 mg/m², carboplatin is administered at a dose in mg to target area under the curve (AUC) of 5 mg/mL/min with maximum dose of 750 mg and etoposide is administered at a dose of 100 mg/m² or 75 mg/m².

In one embodiment of the methods provided above ifosfamide is administered at a dose of 5000 mg/m², carboplatin is administered at a dose in mg to target area under the curve (AUC) of 5 mg/mL/min with maximum dose of 750 mg and etoposide is administered at a dose of 100 mg/m².

In one embodiment, ifosfamide is administered at a dose of about 5000 mg/m² (e.g., 5000 mg/m²±50 mg/m², ±100 mg/m², ±200 mg/m², ±300 mg/m², ±400 mg/m², or ±500 mg/m²), about 4000 mg/m² (e.g., 4000 mg/m²±40 mg/m², ±50 mg/m², ±100 mg/m², ±200 mg/m², ±300 mg/m², or ±400 mg/m²), or about 1666 mg/m² (e.g., 1666 mg/m²±25 mg/m², ±50 mg/m², ±100 mg/m², or ±166.6 mg/m²), carboplatin is administered at a dose of about 5×(25+CreatinineClearance (CrCl)) mg with maximum dose of about 750 mg (e.g., 750 mg±10 mg, ±25 mg, ±50 mg, or ±75 mg), and etoposide is administered at a dose of about 100 mg/m² (e.g., 100 mg/m²±1 mg/m², ±2.5 mg/m², ±5 mg/m², or ±10 mg/m²) or 75 mg/m² (e.g., 0.5 mg/m²±1 mg/m², ±2.5 mg/m², ±5 mg/m², or ±7.5 mg/m²). In one embodiment, (a) the subject is male, and CrCl is calculated using the formula CrCl=([140−age]×[weight in kg])/(72×[serum creatinine in mg/dL]); or (b) the subject is female, and CrCl is calculated using the formula CrCl=0.85×([140−age]×[weight in kg])/(72×[serum creatinine in mg/dL]). In one embodiment, (a) the subject has CrCl<about 60 mL/min, and each single dose of ifosfamide is reduced to 4000 mg/m²; and/or (b) the subject has CrCl<about 50 mL/min, and each dose of etoposide is reduced to about 75 mg/m². In one embodiment, the subject is administered or is to be administered ifosfamide in an outpatient setting, and ifosfamide is administered at a dose of about 1666 mg/m² (e.g., 1666 mg/m²±25 mg/m², ±50 mg/m², ±100 mg/m², or ±166.6 mg/m²).

In one embodiment, ifosfamide and carboplatin are administered on Day 2 of the first and second dosing cycle and etoposide is administered on each of Days 1, 2 and 3 of the first and second dosing cycle.

In one aspect, the invention features a method of treating a subject having a CD20-positive cell proliferative disorder, e.g., a B cell proliferative disorder (e.g., an NHL (e.g., a relapsed and/or refractory NHL, a DLBCL (e.g., a relapsed and/or refractory DLBCL), a FL (e.g., a relapsed and/or refractory FL or a transformed FL), or an MCL (e.g., a relapsed or refractory MCL)), or a CNSL), comprising administering to the subject in a first dosing cycle:
(a) about 2.5 mg (e.g., 2.5 mg±0.01 mg, ±0.02 mg, ±0.03 mg, ±0.05 mg, ±0.1 mg, ±0.2 mg, or ±0.25 mg) as the first dose (C1D1) of a bispecific antibody that binds to CD20 and CD3 and about 10 mg (e.g., 10 mg±0.05 mg, ±0.1 mg, ±0.2 mg, ±0.3 mg, ±0.5 mg, ±0.75 mg, or ±1 mg) as the second dose (C1D2) of the bispecific antibody that binds to CD20 and CD3;
(b) a single dose (C1D1) of about 1000 mg obinutuzumab (e.g., 1000 mg±5 mg, ±10 mg, ±20 mg, ±30 mg, ±50 mg, ±75 mg, or ±100 mg); and
(c) a single dose (C1D1) of about 5000 mg/m² (e.g., 5000 mg/m²±50 mg/m², ±100 mg/m², ±200 mg/m², ±300 mg/m², ±400 mg/m², or ±500 mg/m²) ifosfamide, a single dose (C1D1) of carboplatin at a dose in mg to target area under the curve (AUC) of 5 mg/mL/min with maximum dose of 750 mg and a first (C1D1), second (C1D2) and third (C1D3) dose of about 100 mg/m² etoposide;
and administering to the subject in a second dosing cycle:
(a) a single dose (C2D1) of about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg) of a bispecific antibody that binds to CD20 and CD3;
(b) a single dose (C2D1) of about 375 mg/m² (e.g., 375 mg/m²±5 mg/m², ±10 mg/m², ±25 mg/m², or ±37.5 mg/m²) rituximab; and
(c) a single dose (C1D1) of about 5000 mg/m² (e.g., 5000 mg/m²±50 mg/m², ±100 mg/m², ±200 mg/m², ±300 mg/m², ±400 mg/m², or ±500 mg/m²) ifosfamide, a single dose (C1D1) of carboplatin at a dose in mg to target area under the curve (AUC) of 5 mg/mL/min with maximum dose of 750 mg and a first (C1D1), second (C1D2) and third (C1D3) dose of about 100 mg/m² etoposide.

In one aspect, the invention features a method of treating a subject having a CD20-positive cell proliferative disorder, e.g., a B cell proliferative disorder (e.g., an NHL (e.g., a relapsed and/or refractory NHL, a DLBCL (e.g., a relapsed and/or refractory DLBCL), a FL (e.g., a relapsed and/or refractory FL or a transformed FL), or an MCL (e.g., a relapsed or refractory MCL)), or a CNSL), comprising administering to the subject:
(a) about 2.5 mg (e.g., 2.5 mg±0.01 mg, ±0.02 mg, ±0.03 mg, ±0.05 mg, ±0.1 mg, ±0.2 mg, or ±0.25 mg) as the first dose (C1D1) of a bispecific antibody that binds to CD20 and CD3 on Day 8, about 10 mg (e.g., 10 mg±0.05 mg, ±0.1 mg, ±0.2 mg, ±0.3 mg, ±0.5 mg, ±0.75 mg, or ±1 mg) as the second dose (C1D2) of the bispecific antibody that binds to CD20 and CD3 on Day 15 of the first dosing cycle and about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg) as the first dose (C2D1) on Day 8 of the second dosing cycle;
(b) a single dose (C1D1) of about 1000 mg (e.g., 1000 mg±5 mg, ±10 mg, ±20 mg, ±30 mg, ±50 mg, ±75 mg, or ±100 mg) obinutuzumab on Day 1 of the first dosing cycle and a single dose (C2D1) of about 375 mg/m² (e.g., 375 mg/m²±5 mg/m², ±10 mg/m², ±25 mg/m², or ±37.5 mg/m²) rituximab on Day 1 of the second dosing cycle; and
(c) a single dose (C1D1) of about 5000 mg/m² (e.g., 5000 mg/m²±50 mg/m², ±100 mg/m², ±200 mg/m², ±300 mg/m², ±400 mg/m², or ±500 mg/m²) ifosfamide on Day 2 of the first and second dosing cycle, a single dose (C1D1) of carboplatin at a dose in mg to target area under the curve (AUC) of 5 mg/mL/min with maximum dose of 750 mg on Day 2 of the first and second dosing cycle, and a first (C1D1), second (C1D2) and third (C1D3) dose of about 100 mg/m² etoposide on Days 1, 2 and 3 of the first and second dosing cycle.

In some embodiments, the first and second dosing cycles are 14-day (e.g., 14±3 days) dosing cycles. In some embodiments, the first and second dosing cycles are 21-day (e.g., 21±3 days) dosing cycles. In particular embodiments, the first and second dosing cycles are 21-day dosing cycles.

In some embodiments, the dosing regimen comprises one or more additional dosing cycles. In some embodiments, the dosing regimen comprises three dosing cycles in total. In some embodiments, the additional dosing cycles are 14-day (e.g., 14±3 days) dosing cycles. In some embodiments, the additional dosing cycles are 21-day (e.g., 21±3 days) dosing cycles. In one embodiment, the method provided herein comprises three 21-day dosing cycles.

In one embodiment, the one or more additional dosing cycles comprise:
(a) an additional single dose of the bispecific antibody that binds to CD20 and CD3,
(b) an additional single dose of the anti-CD20 antibody, and
(c) an additional single dose of ifosfamide, an additional single dose of carboplatin and an additional first, second and third dose of etoposide.

In one embodiment, the additional single dose of the bispecific antibody is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one embodiment, the anti-CD20 antibody is rituximab. In one embodiment, the additional single dose of rituximab is about 375 mg/m² (e.g., 375 mg/m²±5 mg/m², ±10 mg/m², ±25 mg/m², or ±37.5 mg/m²). In one embodiment, the additional single dose of rituximab is administered on Day 1 of the additional dosing cycle.

In one embodiment, the additional single dose of ifosfamide is about 5000 mg/m² (e.g., 5000 mg/m²±50 mg/m², ±100 mg/m², ±200 mg/m², ±300 mg/m², ±400 mg/m², or ±500 mg/m²), about 4000 mg/m² (e.g., 4000 mg/m²±40 mg/m², ±50 mg/m², ±100 mg/m², ±200 mg/m², ±300 mg/m², or ±400 mg/m²), or about 1666 mg/m² (e.g., 1666 mg/m²±25 mg/m², ±50 mg/m², ±100 mg/m², or ±166.6 mg/m²), the additional single dose of carboplatin is in mg to target area under the curve (AUC) of about 5 mg/mL/min (e.g., 5 mg/mL/min±0.05 mg/mL/min, ±0.1 mg/mL/min, ±0.25 mg/mL/min, or ±0.5 mg/mL/min) with maximum dose of about 750 mg (e.g., 750 mg±10 mg, ±25 mg, ±50 mg, or ±75 mg) and the additional first, second and third dose of etoposide is 100 mg/m² (e.g., 100 mg/m²+1 mg/m², 2.5 mg/m², ±5 mg/m², or ±10 mg/m²) or 75 mg/m² (e.g., 0.5 mg/m²±1 mg/m², ±2.5 mg/m², ±5 mg/m², or ±7.5 mg/m²). In one embodiment, the additional single dose of ifosfamide is 5000 mg/m², 4000 mg/m² or 1666 mg/m², the additional single dose of carboplatin is in mg to target area under the curve (AUC) of 5 mg/mL/min with maximum dose of 750 mg and the additional first, second and third dose of etoposide is 100 mg/m² or 75 mg/m². In one embodiment, ifosfamide is administered at a dose of 5000 mg/m², carboplatin is administered at a dose in mg to target area under the curve (AUC) of 5 mg/mL/min with maximum dose of 750 mg and etoposide is administered at a dose of 100 mg/m².

In one embodiment, the additional single dose of ifosfamide is about 5000 mg/m² (e.g., 5000 mg/m²±50 mg/m², ±100 mg/m², ±200 mg/m², ±300 mg/m², ±400 mg/m², or ±500 mg/m²), about 4000 mg/m² (e.g., 4000 mg/m²±40 mg/m², ±50 mg/m², ±100 mg/m², ±200 mg/m², ±300 mg/m², or ±400 mg/m²), or about 1666 mg/m² (e.g., 1666 mg/m²±25 mg/m², ±50 mg/m², ±100 mg/m², or ±166.6 mg/m²), the additional single dose of carboplatin is about 5×(25+CreatinineClearance (CrCl)) mg with maximum dose of about 750 mg (e.g., 750 mg±10 mg, ±25 mg, ±50 mg, or ±75 mg), and the additional single dose of etoposide is about 100 mg/m² (e.g., 100 mg/m²±1 mg/m², ±2.5 mg/m², ±5 mg/m², or ±10 mg/m²) or 75 mg/m² (e.g., 0.5 mg/m²±1 mg/m², ±2.5 mg/m², ±5 mg/m², or ±7.5 mg/m²). In one embodiment, (a) the subject is male, and CrCl is calculated using the formula CrCl=([140−age]×[weight in kg])/(72×[serum creatinine in mg/dL]); or (b) the subject is female, and CrCl is calculated using the formula CrCl=0.85×([140−age]×[weight in kg])/(72×[serum creatinine in mg/dL]). In one embodiment, (a) the subject has CrCl<about 60 mL/min, and the additional single dose of ifosfamide is reduced to 4000 mg/m²; and/or (b) the subject has CrCl<about 50 mL/min, and the additional single dose of etoposide is reduced to about 75 mg/m². In one embodiment, the subject is administered or is to be administered the additional single dose of ifosfamide in an outpatient setting, and the additional single dose of ifosfamide is about 1666 mg/m² (e.g., 1666 mg/m²±25 mg/m², ±50 mg/m², ±100 mg/m², or ±166.6 mg/m²).

In one embodiment, ifosfamide and carboplatin are administered on Day 2 of the additional dosing cycle and etoposide is administered on each of Days 1, 2 and 3 of the additional dosing cycle.

In one aspect, the invention features a method of treating a subject having a CD20-positive cell proliferative disorder, e.g., a B cell proliferative disorder (e.g., an NHL (e.g., a relapsed and/or refractory NHL, a DLBCL (e.g., a relapsed and/or refractory DLBCL), a FL (e.g., a relapsed and/or refractory FL or a transformed FL), or an MCL (e.g., a relapsed or refractory MCL)), or a CNSL), comprising administering to the subject in a first dosing cycle:
(a) about 2.5 mg (e.g., 2.5 mg±0.01 mg, ±0.02 mg, ±0.03 mg, ±0.05 mg, ±0.1 mg, ±0.2 mg, or ±0.25 mg) as the first dose (C1D1) of a bispecific antibody that binds to CD20 and CD3 and about 10 mg (e.g., 10 mg±0.05 mg, ±0.1 mg, ±0.2 mg, ±0.3 mg, ±0.5 mg, ±0.75 mg, or ±1 mg) as the second dose (C1D2) of the bispecific antibody that binds to CD20 and CD3;
(b) a single dose (C1D1) of obinutuzumab; and
(c) a single dose (C1D1) of ifosfamide, a single dose (C1D1) of carboplatin and a first (C1D1), second (C1D2) and third (C1D3) dose of etoposide;
and administering to the subject in a second and third dosing cycle:
(a) a single dose (C2D1 in Cycle 2 and C3D1 in Cycle 3) of about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg) of a bispecific antibody that binds to CD20 and CD3;
(b) a single dose (C2D1 in Cycle 2 and C3D1 in Cycle 3) of rituximab; and
(c) a single dose (C2D1) of ifosfamide, a single dose (C2D1 in Cycle 2 and C3D1 in Cycle 3) of carboplatin and a first (C2D1 in Cycle 2 and C3D1 in Cycle 3), second (C2D2 in Cycle 2 and C3D2 in Cycle 3) and third (C2D3 in Cycle 2 and C3D3 in Cycle 3) dose of etoposide.

In one aspect, the invention features a method of treating a subject having a CD20-positive cell proliferative disorder, e.g., a B cell proliferative disorder (e.g., an NHL (e.g., a relapsed and/or refractory NHL, a DLBCL (e.g., a relapsed and/or refractory DLBCL), a FL (e.g., a relapsed and/or refractory FL or a transformed FL), or an MCL (e.g., a relapsed or refractory MCL)), or a CNSL), comprising administering to the subject in a first dosing cycle:
(a) about 2.5 mg (e.g., 2.5 mg±0.01 mg, ±0.02 mg, ±0.03 mg, ±0.05 mg, ±0.1 mg, ±0.2 mg, or ±0.25 mg) as the first dose (C1D1) of a bispecific antibody that binds to CD20 and CD3 and about 10 mg (e.g., 10 mg±0.05 mg, ±0.1 mg, ±0.2 mg, ±0.3 mg, ±0.5 mg, ±0.75 mg, or ±1 mg) as the second dose (C1D2) of the bispecific antibody that binds to CD20 and CD3;
(b) a single dose (C1D1) of about 1000 mg (e.g., 1000 mg±5 mg, ±10 mg, ±20 mg, ±30 mg, ±50 mg, ±75 mg, or ±100 mg) obinutuzumab; and
(c) a single dose (C1D1) of about 5000 mg/m² (e.g., 5000 mg/m²±50 mg/m², ±100 mg/m², ±200 mg/m², ±300 mg/m², ±400 mg/m², or ±500 mg/m²) ifosfamide, a single dose (C1D1) of carboplatin at a dose in mg to target area under the curve (AUC) of 5 mg/mL/min with maximum dose of 750 mg and a first (C1D1), second (C1D2) and third (C1D3) dose of about 100 mg/m² etoposide;
and administering to the subject in a second and third dosing cycle:
(a) a single dose (C2D1 in Cycle 2 and C3D1 in Cycle 3) of about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg) of a bispecific antibody that binds to CD20 and CD3;
(b) a single dose (C2D1 in Cycle 2 and C3D1 in Cycle 3) of about 375 mg/m² (e.g., 375 mg/m²±5 mg/m², ±10 mg/m², ±25 mg/m², or ±37.5 mg/m²) rituximab; and
(c) a single dose (C2D1 in Cycle 2 and C3D1 in Cycle 3) of about 5000 mg/m² (e.g., 5000 mg/m²±50 mg/m², ±100 mg/m², ±200 mg/m², ±300 mg/m², ±400 mg/m², or ±500 mg/m²) ifosfamide, a single dose (C2D1 in Cycle 2 and C3D1 in Cycle 3) of carboplatin at a dose in mg to target area under the curve (AUC) of 5 mg/mL/min with maximum dose of 750 mg and a first (C2D1 in Cycle 2 and C3D1 in Cycle 3), second (C2D2 in Cycle 2 and C3D2 in Cycle 3) and third (C2D3 in Cycle 2 and C3D3 in Cycle 3) dose of about 100 mg/m² etoposide.

In one aspect, the invention features a method of treating a subject having a CD20-positive cell proliferative disorder, e.g., a B cell proliferative disorder (e.g., an NHL (e.g., a relapsed and/or refractory NHL, a DLBCL (e.g., a relapsed and/or refractory DLBCL), a FL (e.g., a relapsed and/or refractory FL or a transformed FL), or an MCL (e.g., a relapsed or refractory MCL)), or a CNSL), comprising administering to the subject:
(a) about 2.5 mg (e.g., 2.5 mg±0.01 mg, ±0.02 mg, ±0.03 mg, ±0.05 mg, ±0.1 mg, ±0.2 mg, or ±0.25 mg) as the first dose (C1D1) of a bispecific antibody that binds to CD20 and CD3 on Day 8, about 10 mg (e.g., 10 mg±0.05 mg, ±0.1 mg, ±0.2 mg, ±0.3 mg, ±0.5 mg, ±0.75 mg, or ±1 mg) as the second dose (C1D2) of the bispecific antibody that binds to CD20 and CD3 on Day 15 of the first dosing cycle and about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg) as the first dose (C2D1) on Day 8 of the second and third dosing cycle;
(b) a single dose (C1D1) of about 1000 mg (e.g., 1000 mg±5 mg, ±10 mg, ±20 mg, ±30 mg, ±50 mg, ±75 mg, or ±100 mg) obinutuzumab on Day 1 of the first dosing cycle and a single dose (C2D1) of about 375 mg/m² (e.g., 375 mg/m²±5 mg/m², ±10 mg/m², ±25 mg/m², or ±37.5 mg/m²) rituximab on Day 1 of the second and third dosing cycle; and
(c) a single dose (C1D1) of about 5000 mg/m² (e.g., 5000 mg/m²±50 mg/m², ±100 mg/m², ±200 mg/m², ±300 mg/m², ±400 mg/m², or ±500 mg/m²) ifosfamide on Day 2 of the first and second dosing cycle, a single dose (C1D1) of carboplatin at a dose in mg to target area under the curve (AUC) of 5 mg/mL/min with maximum dose of 750 mg on Day 2 of all three dosing cycles, and a first (C1D1), second (C1D2) and third (C1D3) dose of about 100 mg/m² etoposide on Days 1, 2 and 3 of all three dosing cycles.

In some embodiments, the dosing cycles are 14-day (e.g., 14±3 days) dosing cycles. In some embodiments, the dosing cycles are 21-day (e.g., 21±3 days) dosing cycles. In particular embodiments, the dosing cycles are 21-day dosing cycles.

In some embodiments, the methods featured by the invention further comprises administering to the subject one or more additional therapeutic agents. In some embodiments, the one or more additional therapeutic agents is tocilizumab. In one embodiment, the weight of the subject is greater than or equal to about 30 kg, and tocilizumab is administered at a dose of about 8 mg/kg (e.g., 8 mg/kg±0.05 mg/kg, ±0.1 mg/kg, ±0.25 mg/kg, ±0.5 mg/kg, or ±0.8 mg/kg). In one embodiment, the weight of the subject is less than 30 kg, and tocilizumab is administered at a dose of about 12 mg/kg (e.g., 12 mg/kg±0.05 mg/kg, ±0.1 mg/kg, ±0.25 mg/kg, ±0.5 mg/kg, ±0.75 mg/kg, ±1 mg/kg, or ±1.2 mg/kg). In some embodiments, the maximum dose of tocilizumab is about 800 mg (e.g., 800 mg±10 mg, ±25 mg, ±50 mg, or ±80 mg).

In some embodiments, the one or more additional therapeutic agents is a corticosteroid. In some embodiments, the corticosteroid comprises prednisone, prednisolone, methylprednisolone, or dexamethasone. In one embodiment, dexamethasone is administered intravenously at a dose of about 20 mg (e.g., 20 mg±0.1 mg, ±0.25 mg, ±0.5 mg, ±1 mg, ±1.5 mg, or ±2 mg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the bispecific antibody. In one embodiment, dexamethasone is administered intravenously at a dose of about 20 mg (e.g., 20 mg±0.1 mg, ±0.25 mg, ±0.5 mg, ±1 mg, ±1.5 mg, or ±2 mg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of obinutuzumab. In one embodiment, wherein methylprednisolone is administered intravenously at a dose of about 80 mg (e.g., 80 mg±0.5 mg, ±1 mg, ±1.5 mg, ±2 mg, ±4 mg, ±6 mg, or ±8 mg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the bispecific antibody. In one embodiment, methylprednisolone is administered intravenously at a dose of about 80 mg (e.g., 80 mg±0.5 mg, ±1 mg, ±1.5 mg, ±2 mg, ±4 mg, ±6 mg, or ±8 mg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of obinutuzumab. In one embodiment, prednisone is administered orally at a dose of about 100 mg (e.g., 100 mg±0.5 mg, ±1 mg, ±1.5 mg, ±2 mg, ±4 mg, ±6 mg, ±8 mg, or ±10 mg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the bispecific antibody. In one embodiment, prednisolone is administered intravenously at a dose of about 100 mg (e.g., 100 mg±0.5 mg, ±1 mg, ±1.5 mg, ±2 mg, ±4 mg, ±6 mg, ±8 mg, or ±10 mg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the bispecific antibody.

In some embodiments, one or more additional therapeutic agents is an antihistamine. In some embodiments, the antihistamine is diphenhydramine. In one embodiment, diphenhydramine is administered orally or intravenously at a dose of about 50 mg (e.g., 50 mg±0.5 mg, ±1 mg, ±1.5 mg, ±2 mg, ±3 mg, ±4 mg, or ±5 mg) at least about 30 minutes (i.e., at least 30 minutes±3 minutes; e.g., at least about 1, 2, 3, 4, 5, 6, 7, 8, 9, 10, 11, 12, 18, 24, 36, 48 hours, or more) prior to the administration of any dose of the bispecific antibody.

In some embodiments, the one or more additional therapeutic agents comprises allopurinol and rasburicase.

In some embodiments, the one or more additional therapeutic agents is an antipyretic. In one embodiment, the antipyretic is acetaminophen or paracetamol. In one embodiment, acetaminophen or paracetamol is administered orally at a dose of between about 500 mg to about 1000 mg (e.g., 1000 mg±5 mg, ±10 mg, ±20 mg, ±30 mg, ±50 mg, ±75 mg, or ±100 mg) at least about 30 minutes (i.e., at least 30 minutes±3 minutes; e.g., at least about 1, 2, 3, 4, 5, 6, 7, 8, 9, 10, 11, 12, 18, 24, 36, 48 hours, or more) prior to the administration of any dose of the bispecific antibody. In one embodiment, acetaminophen or paracetamol is administered orally at a dose of between about 500 mg to about 1000 mg (e.g., 1000 mg±5 mg, ±10 mg, ±20 mg, ±30 mg, ±50 mg, ±75 mg, or ±100 mg) at least about 30 minutes (i.e., at least 30 minutes±3 minutes; e.g., at least about 1, 2, 3, 4, 5, 6, 7, 8, 9, 10, 11, 12, 18, 24, 36, 48 hours, or more) prior to the administration of any dose of obinutuzumab.

In some embodiments, the one or more additional therapeutic agents comprises granulocyte colony-stimulating factor (G-CSF). In one embodiment, G-CSF is administered between about one day and about two days (e.g., 24, 26, 28, 30, 32, 36, 38, 40, 42, 44, 46, or 48 hours) after administration of any dose of rituximab, ifosfamide, carboplatin, and/or etoposide.

In some embodiments, the one or more additional therapeutic agents is mesna. In one embodiment, mesna is administered at a dose of about 5000 mg/m² (e.g., 5000 mg/m²±50 mg/m², ±100 mg/m², ±200 mg/m², ±300 mg/m², ±400 mg/m², or ±500 mg/m²), about 4000 mg/m² (e.g., 4000 mg/m²±40 mg/m², ±50 mg/m², ±100 mg/m², ±200 mg/m², ±300 mg/m², or ±400 mg/m²), or about 1666 mg/m² (e.g., 1666 mg/m²±25 mg/m², ±50 mg/m², ±100 mg/m², or ±166.6 mg/m²) intravenously.

In one embodiment, the bispecific antibody comprises at least one Fab molecule which specifically binds to CD20 comprising the following six hypervariable regions (HVRs):
(i) an HVR-H1 comprising the amino acid sequence of YSWIN (SEQ ID NO: 1);
(ii) an HVR-H2 comprising the amino acid sequence of RIFPGDGDTDYNGKFKG (SEQ ID NO: 2);
(iii) an HVR-H3 comprising the amino acid sequence of NVFDGYWLVY (SEQ ID NO:3);
(iv) an HVR-L1 comprising the amino acid sequence of RSSKSLLHSNGITYLY (SEQ ID NO: 4);
(v) an HVR-L2 comprising the amino acid sequence of QMSNLVS (SEQ ID NO: 5); and
(vi) an HVR-L3 comprising the amino acid sequence of AQNLELPYT (SEQ ID NO: 6).

In one embodiment, the bispecific antibody comprises at least one Fab molecule which specifically binds to CD20 comprising (a) a heavy chain variable (VH) domain comprising an amino acid sequence having at least 95% sequence identity to the amino acid sequence of SEQ ID NO: 7; (b) a light chain variable (VL) domain comprising an amino acid sequence having at least 95% sequence identity to the amino acid sequence of SEQ ID NO: 8; or (c) a VH domain as in (a) and a VL domain as in (b).

In one embodiment, the Fab molecule which specifically binds to CD20 comprises (a) a VH domain comprising an amino acid sequence of SEQ ID NO: 7 and (b) a VL domain comprising an amino acid sequence of SEQ ID NO: 8.

In one embodiment, the bispecific antibody comprises at least one Fab molecule which specifically binds to CD3 comprising the following six HVRs:
(i) an HVR-H1 comprising the amino acid sequence of TYAMN (SEQ ID NO: 9);
(ii) an HVR-H2 comprising the amino acid sequence of RIRSKYNNYATYYADSVKG (SEQ ID NO: 10);
(iii) an HVR-H3 comprising the amino acid sequence of HGNFGNSYVSWFAY (SEQ ID NO: 11);
(iv) an HVR-L1 comprising the amino acid sequence of GSSTGAVTTSNYAN (SEQ ID NO: 12);
(v) an HVR-L2 comprising the amino acid sequence of GTNKRAP (SEQ ID NO: 13); and
(vi) an HVR-L3 comprising the amino acid sequence of ALWYSNLWV (SEQ ID NO: 14).

In one embodiment, the bispecific antibody comprises at least one Fab molecule which specifically binds to CD3 comprising (a) a heavy chain variable (VH) domain comprising an amino acid sequence having at least 95% sequence identity to the amino acid sequence of SEQ ID NO: 15; (b) a light chain variable (VL) domain comprising an amino acid sequence having at least 95% sequence identity to the amino acid sequence of SEQ ID NO: 16; or (c) a VH domain as in (a) and a VL domain as in (b).

In one embodiment, the Fab molecule which specifically binds to CD3 comprises (a) a VH domain comprising an amino acid sequence of SEQ ID NO: 15 and (b) a VL domain comprising an amino acid sequence of SEQ ID NO: 16.

In one embodiment, the bispecific antibody is bivalent for CD20 and monovalent for CD3. In one embodiment, the bispecific antibody comprises two Fab molecules which specifically bind to CD20 and one Fab molecule which specifically binds to CD3. In one embodiment the bispecific antibody is a humanized antibody.

In one aspect, the invention features a method of treating a subject having a CD20-positive cell proliferative disorder, e.g., a B cell proliferative disorder (e.g., an NHL (e.g., a relapsed and/or refractory NHL, a DLBCL (e.g., a relapsed and/or refractory DLBCL), a FL (e.g., a relapsed and/or refractory FL or a transformed FL), or an MCL (e.g., a relapsed or refractory MCL)), or a CNSL), comprising administering to the subject an effective amount of:
(a) a bispecific antibody that binds to CD20 and CD3 comprising at least one Fab molecule which specifically binds to CD20 comprising the following six hypervariable regions (HVRs):
(i) an HVR-H1 comprising the amino acid sequence of YSWIN (SEQ ID NO: 1);
(ii) an HVR-H2 comprising the amino acid sequence of RIFPGDGDTDYNGKFKG (SEQ ID NO: 2);
(iii) an HVR-H3 comprising the amino acid sequence of NVFDGYWLVY (SEQ ID NO:3);
(iv) an HVR-L1 comprising the amino acid sequence of RSSKSLLHSNGITYLY (SEQ ID NO: 4);
(v) an HVR-L2 comprising the amino acid sequence of QMSNLVS (SEQ ID NO: 5); and
(vi) an HVR-L3 comprising the amino acid sequence of AQNLELPYT (SEQ ID NO: 6); and and at least one Fab molecule which specifically binds to CD3 comprising the following six HVRs:
(i) an HVR-H1 comprising the amino acid sequence of TYAMN (SEQ ID NO: 9);
(ii) an HVR-H2 comprising the amino acid sequence of RIRSKYNNYATYYADSVKG (SEQ ID NO: 10);
(iii) an HVR-H3 comprising the amino acid sequence of HGNFGNSYVSWFAY (SEQ ID NO: 11);
(iv) an HVR-L1 comprising the amino acid sequence of GSSTGAVTTSNYAN (SEQ ID NO: 12);
(v) an HVR-L2 comprising the amino acid sequence of GTNKRAP (SEQ ID NO: 13); and
(vi) an HVR-L3 comprising the amino acid sequence of ALWYSNLWV (SEQ ID NO: 14);
(b) an anti-CD20 antibody; and
(c) one or more chemotherapeutic agents selected from ifosfamide, carboplatin and/or etoposide in a dosing regimen comprising at least a first dosing cycle and a second dosing cycle.

In one embodiment, the first dosing cycle comprises a first dose (C1D1) of the bispecific antibody and a second dose (C1D2) of the bispecific antibody, wherein the C1D1 of the bispecific antibody is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of the bispecific antibody is about 10 mg (e.g., 10 mg±0.05 mg, ±0.1 mg, ±0.2 mg, ±0.3 mg, ±0.5 mg, ±0.75 mg, or ±1 mg); and the second dosing cycle comprises a single dose (C2D1) of the bispecific antibody, wherein the C2D1 of the bispecific antibody is about 10 mg (e.g., 10 mg±0.05 mg, ±0.1 mg, ±0.2 mg, ±0.3 mg, ±0.5 mg, ±0.75 mg, or ±1 mg), about 16 mg (e.g., 16 mg±0.05 mg, ±0.1 mg, ±0.2 mg, ±0.3 mg, ±0.5 mg, ±0.75 mg, ±1 mg, ±1.5 mg, or ±1.6 mg), or about 30 mg (e.g., 30 mg ±0.1 mg, ±0.2 mg, ±0.3 mg, ±0.5 mg, ±0.75 mg, ±1 mg, ±1.5 mg, ±2 mg, or ±3 mg). In particular embodiments, the C1D1 of the bispecific antibody is about 2.5 mg (e.g., 2.5 mg±0.01 mg, ±0.02 mg, ±0.03 mg, ±0.05 mg, ±0.1 mg, ±0.2 mg, or ±0.25 mg) and the C1D2 of the bispecific antibody is about 10 mg (e.g., 10 mg±0.05 mg, ±0.1 mg, ±0.2 mg, ±0.3 mg, ±0.5 mg, ±0.75 mg, or ±1 mg). In particular embodiments, the C2D1 is about 10 mg (e.g., 10 mg±0.05 mg, ±0.1 mg, ±0.2 mg, ±0.3 mg, ±0.5 mg, ±0.75 mg, or ±1 mg). In particular embodiments, the C2D1 of the bispecific antibody is about 16 mg (e.g., 16 mg±0.05 mg, ±0.1 mg, ±0.2 mg, ±0.3 mg, ±0.5 mg, ±0.75 mg, ±1 mg, ±1.5 mg, or ±1.6 mg). In particular embodiments, the C2D1 of the bispecific antibody is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one embodiment, the first dosing cycle comprises a first dose (C1D1) of the bispecific antibody and a second dose (C1D2) of the bispecific antibody, wherein the C1D1 of the bispecific antibody is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of the bispecific antibody is about 10 mg (e.g., 10 mg±0.05 mg, ±0.1 mg, ±0.2 mg, ±0.3 mg, ±0.5 mg, ±0.75 mg, or ±1 mg); and the second dosing cycle comprises a single dose (C2D1) of the bispecific antibody, wherein the C2D1 of the bispecific antibody is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one embodiment, the first dose (C1D1) of the bispecific antibody and the second dose (C1D2) of the bispecific antibody are administered to the subject on or about Days 8 (±1 day) and 15 (±1 day), respectively, of the first dosing cycle.

In one embodiment, the first dose (C1D1) of the bispecific antibody and the second dose (C1D2) of the bispecific antibody are administered to the subject on Days 8 and 15, respectively, of the first dosing cycle. In some embodiments, the C2D1 of the bispecific antibody is administered to the subject on or about Day 8 (±1 day) of the second dosing cycle. In some embodiments, the C2D1 of the bispecific antibody is administered to the subject on or about Day 8 of the second dosing cycle.

In one embodiment, the anti-CD20 antibody is obinutuzumab and/or rituximab. In one embodiment, the first dosing cycle comprises a single dose (C1D1) of obinutuzumab; and the second dosing cycle comprises a single dose (C2D1) of rituximab.

In one embodiment, the single dose C1D1 of the obinutuzumab is about 1000 mg (e.g., 1000 mg ±5 mg, ±10 mg, ±20 mg, ±30 mg, ±50 mg, ±75 mg, or ±100 mg) and the single dose of rituximab is about 375 mg/m² (e.g., 375 mg/m²±5 mg/m², ±10 mg/m², ±25 mg/m², or ±37.5 mg/m²).

In one embodiment, the anti-CD20 antibody is administered in a dosing regimen comprising at least a first dosing cycle and a second dosing cycle, wherein the first dosing cycle comprises a single dose (C1D1) of obinutuzumab on Day 1; and the second dosing cycle comprises a single dose (C2D1) of rituximab on Day 1.

In one aspect, the invention features a method of treating a subject having a CD20-positive cell proliferative disorder, e.g., a B cell proliferative disorder (e.g., an NHL (e.g., a relapsed and/or refractory NHL, a DLBCL (e.g., a relapsed and/or refractory DLBCL), a FL (e.g., a relapsed and/or refractory FL or a transformed FL), or an MCL (e.g., a relapsed or refractory MCL)), or a CNSL), comprising administering to the subject in a first dosing cycle:
(a) about 2.5 mg (e.g., 2.5 mg±0.01 mg, ±0.02 mg, ±0.03 mg, ±0.05 mg, ±0.1 mg, ±0.2 mg, or ±0.25 mg) as the first dose (C1D1) of a bispecific antibody that binds to CD20 and CD3 and about 10 mg (e.g., 10 mg±0.05 mg, ±0.1 mg, ±0.2 mg, ±0.3 mg, ±0.5 mg, ±0.75 mg, or ±1 mg) as the second dose (C1D2) of the bispecific antibody that binds to CD20 and CD3;
(b) a single dose (C1D1) of obinutuzumab; and
(c) one or more chemotherapeutic agents selected from ifosfamide, carboplatin and/or etoposide;
and administering to the subject in a second dosing cycle:
(a) a single dose (C2D1) of about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg) of a bispecific antibody that binds to CD20 and CD3;
(b) a single dose (C2D1) of rituximab; and
(c) one or more chemotherapeutic agents selected from ifosfamide, carboplatin and/or etoposide, wherein the bispecific antibody that binds to CD20 and CD3 comprises at least one Fab molecule which specifically binds to CD20 comprising the following six hypervariable regions (HVRs):
(i) an HVR-H1 comprising the amino acid sequence of YSWIN (SEQ ID NO: 1);
(ii) an HVR-H2 comprising the amino acid sequence of RIFPGDGDTDYNGKFKG (SEQ ID NO: 2);
(iii) an HVR-H3 comprising the amino acid sequence of NVFDGYWLVY (SEQ ID NO:3);
(iv) an HVR-L1 comprising the amino acid sequence of RSSKSLLHSNGITYLY (SEQ ID NO: 4);
(v) an HVR-L2 comprising the amino acid sequence of QMSNLVS (SEQ ID NO: 5); and
(vi) an HVR-L3 comprising the amino acid sequence of AQNLELPYT (SEQ ID NO: 6);
and at least one Fab molecule which specifically binds to CD3 comprising the following six HVRs:
(i) an HVR-H1 comprising the amino acid sequence of TYAMN (SEQ ID NO: 9);
(ii) an HVR-H2 comprising the amino acid sequence of RIRSKYNNYATYYADSVKG (SEQ ID NO: 10);
(iii) an HVR-H3 comprising the amino acid sequence of HGNFGNSYVSWFAY (SEQ ID NO: 11);
(iv) an HVR-L1 comprising the amino acid sequence of GSSTGAVTTSNYAN (SEQ ID NO: 12);
(v) an HVR-L2 comprising the amino acid sequence of GTNKRAP (SEQ ID NO: 13); and
(vi) an HVR-L3 comprising the amino acid sequence of ALWYSNLWV (SEQ ID NO: 14).

In one embodiment, the single dose C1D1 of the obinutuzumab is about 1000 mg (e.g., 1000 mg ±5 mg, ±10 mg, ±20 mg, ±30 mg, ±50 mg, ±75 mg, or ±100 mg) and the single dose of rituximab is about 375 mg/m² (e.g., 375 mg/m²±5 mg/m², ±10 mg/m², ±25 mg/m², or ±37.5 mg/m²).

In one embodiment, the anti-CD20 antibody is administered in a dosing regimen comprising at least a first dosing cycle and a second dosing cycle, wherein the first dosing cycle comprises a single dose (C1D1) of obinutuzumab on Day 1; and the second dosing cycle comprises a single dose (C2D1) of rituximab on Day 1.

In one embodiment, the first dose (C1D1) of the bispecific antibody and the second dose (C1D2) of the bispecific antibody are administered to the subject on or about Days 8 (±1 day) and 15 (±1 day), respectively, of the first dosing cycle.

In one embodiment, the first dose (C1D1) of the bispecific antibody and the second dose (C1D2) of the bispecific antibody are administered to the subject on Days 8 and 15, respectively, of the first dosing cycle. In some embodiments, the C2D1 of the bispecific antibody is administered to the subject on or about Day 8 (±1 day) of the second dosing cycle. In some embodiments, the C2D1 of the bispecific antibody is administered to the subject on or about Day 8 of the second dosing cycle.

In one embodiment of the methods provided above, step c) comprises all three chemotherapeutic agents.

In one embodiment, the first dosing cycle comprises a single dose (C1D1) of ifosfamide, a single dose (C1D1) of carboplatin and a first (C1D1), second (C1D2) and third (C1D3) dose of etoposide; and the second cycle each comprises a single dose (C2D1) of ifosfamide, a single dose (C2D1) of carboplatin and a first (C2D1), second (C2D2) and third (C2D3) dose of etoposide.

In one aspect, the invention features a method of treating a subject having a CD20-positive cell proliferative disorder, e.g., a B cell proliferative disorder (e.g., an NHL (e.g., a relapsed and/or refractory NHL, a DLBCL (e.g., a relapsed and/or refractory DLBCL), a FL (e.g., a relapsed and/or refractory FL or a transformed FL), or an MCL (e.g., a relapsed or refractory MCL)), or a CNSL), comprising administering to the subject in a first dosing cycle:
(a) about 2.5 mg (e.g., 2.5 mg±0.01 mg, ±0.02 mg, ±0.03 mg, ±0.05 mg, ±0.1 mg, ±0.2 mg, or ±0.25 mg) as the first dose (C1D1) of a bispecific antibody that binds to CD20 and CD3 and about 10 mg (e.g., 10 mg±0.05 mg, ±0.1 mg, ±0.2 mg, ±0.3 mg, ±0.5 mg, ±0.75 mg, or ±1 mg) as the second dose (C1D2) of the bispecific antibody that binds to CD20 and CD3;
(b) a single dose (C1D1) of obinutuzumab; and
(c) a single dose (C1D1) of ifosfamide, a single dose (C1D1) of carboplatin and a first (C1D1), second (C1D2) and third (C1D3) dose of etoposide;
and administering to the subject in a second dosing cycle:
(a) a single dose (C2D1) of about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg) of a bispecific antibody that binds to CD20 and CD3;
(b) a single dose (C2D1) of rituximab; and
(c) a single dose (C2D1) of ifosfamide, a single dose (C2D1) of carboplatin and a first (C2D1), second (C2D2) and third (C2D3) dose of etoposide,
wherein the bispecific antibody that binds to CD20 and CD3 comprises at least one Fab molecule which specifically binds to CD20 comprising the following six hypervariable regions (HVRs):
(i) an HVR-H1 comprising the amino acid sequence of YSWIN (SEQ ID NO: 1);
(ii) an HVR-H2 comprising the amino acid sequence of RIFPGDGDTDYNGKFKG (SEQ ID NO: 2);
(iii) an HVR-H3 comprising the amino acid sequence of NVFDGYWLVY (SEQ ID NO:3);
(iv) an HVR-L1 comprising the amino acid sequence of RSSKSLLHSNGITYLY (SEQ ID NO: 4);
(v) an HVR-L2 comprising the amino acid sequence of QMSNLVS (SEQ ID NO: 5); and
(vi) an HVR-L3 comprising the amino acid sequence of AQNLELPYT (SEQ ID NO: 6), and at least one Fab molecule which specifically binds to CD3 comprising the following six HVRs:
(i) an HVR-H1 comprising the amino acid sequence of TYAMN (SEQ ID NO: 9);
(ii) an HVR-H2 comprising the amino acid sequence of RIRSKYNNYATYYADSVKG (SEQ ID NO: 10);
(iii) an HVR-H3 comprising the amino acid sequence of HGNFGNSYVSWFAY (SEQ ID NO: 11);
(iv) an HVR-L1 comprising the amino acid sequence of GSSTGAVTTSNYAN (SEQ ID NO: 12);
(v) an HVR-L2 comprising the amino acid sequence of GTNKRAP (SEQ ID NO: 13); and
(vi) an HVR-L3 comprising the amino acid sequence of ALWYSNLWV (SEQ ID NO: 14).

In one embodiment, the single dose C1D1 of the obinutuzumab is about 1000 mg (e.g., 1000 mg ±5 mg, ±10 mg, ±20 mg, ±30 mg, ±50 mg, ±75 mg, or ±100 mg) and the single dose of rituximab is about 375 mg/m² (e.g., 375 mg/m²±5 mg/m², ±10 mg/m², ±25 mg/m², or ±37.5 mg/m²).

In one embodiment, the anti-CD20 antibody is administered in a dosing regimen comprising at least a first dosing cycle and a second dosing cycle, wherein the first dosing cycle comprises a single dose (C1D1) of obinutuzumab on Day 1; and the second dosing cycle comprises a single dose (C2D1) of rituximab on Day 1.

In one embodiment, the first dose (C1D1) of the bispecific antibody and the second dose (C1D2) of the bispecific antibody are administered to the subject on or about Days 8 (±1 day) and 15 (±1 day), respectively, of the first dosing cycle.

In one embodiment, the first dose (C1D1) of the bispecific antibody and the second dose (C1D2) of the bispecific antibody are administered to the subject on Days 8 and 15, respectively, of the first dosing cycle. In some embodiments, the C2D1 of the bispecific antibody is administered to the subject on or about Day 8 (±1 day) of the second dosing cycle. In some embodiments, the C2D1 of the bispecific antibody is administered to the subject on or about Day 8 of the second dosing cycle.

In one embodiment of the methods provided above ifosfamide is administered at a dose of 5000 mg/m², 4000 mg/m² or 1666 mg/m². In one embodiment of the methods provided above ifosfamide is administered at a dose of 5000 mg/m². In one embodiment of the methods provided above, carboplatin is administered at a dose in mg to target area under the curve (AUC) of 5 mg/mL/min with maximum dose of 750 mg. In one embodiment of the methods provided above etoposide is administered at a dose of 100 mg/m² or 75 mg/m². In one embodiment of the methods provided above etoposide is administered at a dose of 100 mg/m².

In one embodiment of the methods provided above ifosfamide is administered at a dose of 5000 mg/m², 4000 mg/m² or 1666 mg/m², carboplatin is administered at a dose in mg to target area under the curve (AUC) of 5 mg/mL/min with maximum dose of 750 mg and etoposide is administered at a dose of 100 mg/m² or 75 mg/m².

In one embodiment of the methods provided above ifosfamide is administered at a dose of 5000 mg/m², carboplatin is administered at a dose in mg to target area under the curve (AUC) of 5 mg/mL/min with maximum dose of 750 mg and etoposide is administered at a dose of 100 mg/m².

In one embodiment, ifosfamide and carboplatin are administered on Day 2 of the first and second dosing cycle and etoposide is administered on each of Days 1, 2 and 3 of the first and second dosing cycle.

In one aspect, the invention features a method of treating a subject having a CD20-positive cell proliferative disorder, e.g., a B cell proliferative disorder (e.g., an NHL (e.g., a relapsed and/or refractory NHL, a DLBCL (e.g., a relapsed and/or refractory DLBCL), a FL (e.g., a relapsed and/or refractory FL or a transformed FL), or an MCL (e.g., a relapsed or refractory MCL)), or a CNSL), comprising administering to the subject in a first dosing cycle:
(a) about 2.5 mg (e.g., 2.5 mg±0.01 mg, ±0.02 mg, ±0.03 mg, ±0.05 mg, ±0.1 mg, ±0.2 mg, or ±0.25 mg) as the first dose (C1D1) of a bispecific antibody that binds to CD20 and CD3 and about 10 mg (e.g., 10 mg±0.05 mg, ±0.1 mg, ±0.2 mg, ±0.3 mg, ±0.5 mg, ±0.75 mg, or ±1 mg) as the second dose (C1D2) of the bispecific antibody that binds to CD20 and CD3;
(b) a single dose (C1D1) of about 1000 mg (e.g., 1000 mg±5 mg, ±10 mg, ±20 mg, ±30 mg, ±50 mg, ±75 mg, or ±100 mg) obinutuzumab; and
(c) a single dose (C1D1) of about 5000 mg/m² (e.g., 5000 mg/m²±50 mg/m², ±100 mg/m², ±200 mg/m², ±300 mg/m², ±400 mg/m², or ±500 mg/m²) ifosfamide, a single dose (C1D1) of carboplatin at a dose in mg to target area under the curve (AUC) of 5 mg/mL/min with maximum dose of 750 mg and a first (C1D1), second (C1D2) and third (C1D3) dose of about 100 mg/m² etoposide;
and administering to the subject in a second dosing cycle:
(a) a single dose (C2D1) of about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg) of a bispecific antibody that binds to CD20 and CD3;
(b) a single dose (C2D1) of about 375 mg/m² (e.g., 375 mg/m²±5 mg/m², ±10 mg/m², ±25 mg/m², or ±37.5 mg/m²) rituximab; and
(c) a single dose (C1D1) of about 5000 mg/m² (e.g., 5000 mg/m²±50 mg/m², ±100 mg/m², ±200 mg/m², ±300 mg/m², ±400 mg/m², or ±500 mg/m²) ifosfamide, a single dose (C1D1) of carboplatin at a dose in mg to target area under the curve (AUC) of 5 mg/mL/min with maximum dose of 750 mg and a first (C1D1), second (C1D2) and third (C1D3) dose of about 100 mg/m² etoposide,
wherein the bispecific antibody that binds to CD20 and CD3 comprises at least one Fab molecule which specifically binds to CD20 comprising the following six hypervariable regions (HVRs):
(i) an HVR-H1 comprising the amino acid sequence of YSWIN (SEQ ID NO: 1);
(ii) an HVR-H2 comprising the amino acid sequence of RIFPGDGDTDYNGKFKG (SEQ ID NO: 2);
(iii) an HVR-H3 comprising the amino acid sequence of NVFDGYWLVY (SEQ ID NO:3);
(iv) an HVR-L1 comprising the amino acid sequence of RSSKSLLHSNGITYLY (SEQ ID NO: 4);
(v) an HVR-L2 comprising the amino acid sequence of QMSNLVS (SEQ ID NO: 5); and
(vi) an HVR-L3 comprising the amino acid sequence of AQNLELPYT (SEQ ID NO: 6),
and at least one Fab molecule which specifically binds to CD3 comprising the following six HVRs:
(i) an HVR-H1 comprising the amino acid sequence of TYAMN (SEQ ID NO: 9);
(ii) an HVR-H2 comprising the amino acid sequence of RIRSKYNNYATYYADSVKG (SEQ ID NO: 10),
(iii) an HVR-H3 comprising the amino acid sequence of HGNFGNSYVSWFAY (SEQ ID NO: 11);
(iv) an HVR-L1 comprising the amino acid sequence of GSSTGAVTTSNYAN (SEQ ID NO: 12);
(v) an HVR-L2 comprising the amino acid sequence of GTNKRAP (SEQ ID NO: 13); and
(vi) an HVR-L3 comprising the amino acid sequence of ALWYSNLWV (SEQ ID NO: 14).

In one aspect, the invention features a method of treating a subject having a CD20-positive cell proliferative disorder, e.g., a B cell proliferative disorder (e.g., an NHL (e.g., a relapsed and/or refractory NHL, a DLBCL (e.g., a relapsed and/or refractory DLBCL), a FL (e.g., a relapsed and/or refractory FL or a transformed FL), or an MCL (e.g., a relapsed or refractory MCL)), or a CNSL), comprising administering to the subject:
(a) about 2.5 mg (e.g., 2.5 mg±0.01 mg, ±0.02 mg, ±0.03 mg, ±0.05 mg, ±0.1 mg, ±0.2 mg, or ±0.25 mg) as the first dose (C1D1) of a bispecific antibody that binds to CD20 and CD3 on Day 8, about 10 mg (e.g., 10 mg±0.05 mg, ±0.1 mg, ±0.2 mg, ±0.3 mg, ±0.5 mg, ±0.75 mg, or ±1 mg) as the second dose (C1D2) of the bispecific antibody that binds to CD20 and CD3 on Day 15 of the first dosing cycle and about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg) as the first dose (C2D1) on Day 8 of the second dosing cycle;
(b) a single dose (C1D1) of about 1000 mg (e.g., 1000 mg±5 mg, ±10 mg, ±20 mg, ±30 mg, ±50 mg, ±75 mg, or ±100 mg) obinutuzumab on Day 1 of the first dosing cycle and a single dose (C2D1) of about 375 mg/m² (e.g., 375 mg/m²±5 mg/m², ±10 mg/m², ±25 mg/m², or ±37.5 mg/m²) rituximab on Day 1 of the second dosing cycle; and
(c) a single dose (C1D1) of about 5000 mg/m² (e.g., 5000 mg/m²±50 mg/m², ±100 mg/m², ±200 mg/m², ±300 mg/m², ±400 mg/m², or ±500 mg/m²) ifosfamide on Day 2 of the first and second dosing cycle, a single dose (C1D1) of carboplatin at a dose in mg to target area under the curve (AUC) of 5 mg/mL/min with maximum dose of 750 mg on Day 2 of the first and second dosing cycle, and a first (C1D1), second (C1D2) and third (C1D3) dose of about 100 mg/m² etoposide on Days 1, 2 and 3 of the first and second dosing cycle,
wherein the bispecific antibody that binds to CD20 and CD3 comprises at least one Fab molecule which specifically binds to CD20 comprising the following six hypervariable regions (HVRs):
(i) an HVR-H1 comprising the amino acid sequence of YSWIN (SEQ ID NO: 1);
(ii) an HVR-H2 comprising the amino acid sequence of RIFPGDGDTDYNGKFKG (SEQ ID NO: 2);
(iii) an HVR-H3 comprising the amino acid sequence of NVFDGYWLVY (SEQ ID NO:3);
(iv) an HVR-L1 comprising the amino acid sequence of RSSKSLLHSNGITYLY (SEQ ID NO: 4);
(v) an HVR-L2 comprising the amino acid sequence of QMSNLVS (SEQ ID NO: 5); and
(vi) an HVR-L3 comprising the amino acid sequence of AQNLELPYT (SEQ ID NO: 6);
and at least one Fab molecule which specifically binds to CD3 comprising the following six HVRs:
(i) an HVR-H1 comprising the amino acid sequence of TYAMN (SEQ ID NO: 9);
(ii) an HVR-H2 comprising the amino acid sequence of RIRSKYNNYATYYADSVKG (SEQ ID NO: 10);
(iii) an HVR-H3 comprising the amino acid sequence of HGNFGNSYVSWFAY (SEQ ID NO: 11);
(iv) an HVR-L1 comprising the amino acid sequence of GSSTGAVTTSNYAN (SEQ ID NO: 12);
(v) an HVR-L2 comprising the amino acid sequence of GTNKRAP (SEQ ID NO: 13); and
(vi) an HVR-L3 comprising the amino acid sequence of ALWYSNLWV (SEQ ID NO: 14).

In some embodiments, the first and second dosing cycles are 14-day (e.g., 14±3 days) dosing cycles. In some embodiments, the first and second dosing cycles are 21-day (e.g., 21±3 days) dosing cycles. In particular embodiments, the first and second dosing cycles are 21-day dosing cycles.

In some embodiments, the dosing regimen comprises one or more additional dosing cycles. In some embodiments, the dosing regimen comprises three dosing cycles in total. In some embodiments, the additional dosing cycles are 14-day (e.g., 14±3 days) dosing cycles. In some embodiments, the additional dosing cycles are 21-day (e.g., 21±3 days) dosing cycles. In one embodiment, the method provided herein comprises three 21-day dosing cycles.

In one embodiment, the one or more additional dosing cycles comprise:
(a) an additional single dose of the bispecific antibody that binds to CD20 and CD3,
(b) an additional single dose of the anti-CD20 antibody, and
(c) an additional single dose of ifosfamide, an additional single dose of carboplatin and an additional first, second and third dose of etoposide,
wherein the bispecific antibody that binds to CD20 and CD3 comprises at least one Fab molecule which specifically binds to CD20 comprising the following six hypervariable regions (HVRs):
(i) an HVR-H1 comprising the amino acid sequence of YSWIN (SEQ ID NO: 1);
(ii) an HVR-H2 comprising the amino acid sequence of RIFPGDGDTDYNGKFKG (SEQ ID NO: 2);
(iii) an HVR-H3 comprising the amino acid sequence of NVFDGYWLVY (SEQ ID NO:3);
(iv) an HVR-L1 comprising the amino acid sequence of RSSKSLLHSNGITYLY (SEQ ID NO: 4);
(v) an HVR-L2 comprising the amino acid sequence of QMSNLVS (SEQ ID NO: 5); and
(vi) an HVR-L3 comprising the amino acid sequence of AQNLELPYT (SEQ ID NO: 6),
and at least one Fab molecule which specifically binds to CD3 comprising the following six HVRs:
(i) an HVR-H1 comprising the amino acid sequence of TYAMN (SEQ ID NO: 9);
(ii) an HVR-H2 comprising the amino acid sequence of RIRSKYNNYATYYADSVKG (SEQ ID NO: 10);
(iii) an HVR-H3 comprising the amino acid sequence of HGNFGNSYVSWFAY (SEQ ID NO: 11);
(iv) an HVR-L1 comprising the amino acid sequence of GSSTGAVTTSNYAN (SEQ ID NO: 12);
(v) an HVR-L2 comprising the amino acid sequence of GTNKRAP (SEQ ID NO: 13); and
(vi) an HVR-L3 comprising the amino acid sequence of ALWYSNLWV (SEQ ID NO: 14).

In one embodiment, the additional single dose of the bispecific antibody is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one embodiment, the anti-CD20 antibody is rituximab. In one embodiment, the additional single dose of rituximab is about 375 mg/m² (e.g., 375 mg/m²±5 mg/m², ±10 mg/m², ±25 mg/m², or ±37.5 mg/m²). In one embodiment, the additional single dose of rituximab is administered on Day 1 of the additional dosing cycle.

In one embodiment, the additional single dose of ifosfamide is about 5000 mg/m² (e.g., 5000 mg/m²±50 mg/m², ±100 mg/m², ±200 mg/m², ±300 mg/m², ±400 mg/m², or ±500 mg/m²), about 4000 mg/m² (e.g., 4000 mg/m²±40 mg/m², ±50 mg/m², ±100 mg/m², ±200 mg/m², ±300 mg/m², or ±400 mg/m²), or about 1666 mg/m² (e.g., 1666 mg/m²±25 mg/m², ±50 mg/m², ±100 mg/m², or ±166.6 mg/m²), the additional single dose of carboplatin is in mg to target area under the curve (AUC) of about 5 mg/mL/min (e.g., 5 mg/mL/min±0.05 mg/mL/min, ±0.1 mg/mL/min, ±0.25 mg/mL/min, or ±0.5 mg/mL/min) with maximum dose of about 750 mg (e.g., 750 mg±10 mg, ±25 mg, ±50 mg, or ±75 mg) and the additional first, second and third dose of etoposide is 100 mg/m² (e.g., 100 mg/m²±1 mg/m², 2.5 mg/m², ±5 mg/m², or ±10 mg/m²) or 75 mg/m² (e.g., 0.5 mg/m²±1 mg/m², ±2.5 mg/m², ±5 mg/m², or ±7.5 mg/m²). In one embodiment, the additional single dose of ifosfamide is 5000 mg/m², 4000 mg/m² or 1666 mg/m², the additional single dose of carboplatin is in mg to target area under the curve (AUC) of 5 mg/mL/min with maximum dose of 750 mg and the additional first, second and third dose of etoposide is 100 mg/m² or 75 mg/m². In one embodiment, ifosfamide is administered at a dose of 5000 mg/m², carboplatin is administered at a dose in mg to target area under the curve (AUC) of 5 mg/mL/min with maximum dose of 750 mg and etoposide is administered at a dose of 100 mg/m².

In one embodiment, ifosfamide and carboplatin are administered on Day 2 of the additional dosing cycle and etoposide is administered on each of Days 1, 2 and 3 of the additional dosing cycle.

In one aspect, the invention features a method of treating a subject having a CD20-positive cell proliferative disorder, e.g., a B cell proliferative disorder (e.g., an NHL (e.g., a relapsed and/or refractory NHL, a DLBCL (e.g., a relapsed and/or refractory DLBCL), a FL (e.g., a relapsed and/or refractory FL or a transformed FL), or an MCL (e.g., a relapsed or refractory MCL)), or a CNSL), comprising administering to the subject in a first dosing cycle:
(a) about 2.5 mg (e.g., 2.5 mg±0.01 mg, ±0.02 mg, ±0.03 mg, ±0.05 mg, ±0.1 mg, ±0.2 mg, or ±0.25 mg) as the first dose (C1D1) of a bispecific antibody that binds to CD20 and CD3 and about 10 mg (e.g., 10 mg±0.05 mg, ±0.1 mg, ±0.2 mg, ±0.3 mg, ±0.5 mg, ±0.75 mg, or ±1 mg) as the second dose (C1D2) of the bispecific antibody that binds to CD20 and CD3;
(b) a single dose (C1D1) of obinutuzumab; and
(c) a single dose (C1D1) of ifosfamide, a single dose (C1D1) of carboplatin and a first (C1D1), second (C1D2) and third (C1D3) dose of etoposide;
and administering to the subject in a second and third dosing cycle:
(a) a single dose (C2D1 in Cycle 2 and C3D1 in Cycle 3) of about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg) of a bispecific antibody that binds to CD20 and CD3;
(b) a single dose (C2D1 in Cycle 2 and C3D1 in Cycle 3) of rituximab; and
(c) a single dose (C2D1 in Cycle 2 and C3D1 in Cycle 3) of ifosfamide, a single dose (C2D1 in Cycle 2 and C3D1 in Cycle 3) of carboplatin and a first (C2D1 in Cycle 2 and C3D1 in Cycle 3), second (C2D2 in Cycle 2 and C3D2 in Cycle 3) and third (C2D3 in Cycle 2 and C3D3 in Cycle 3) dose of etoposide, wherein the bispecific antibody that binds to CD20 and CD3 comprises at least one Fab molecule which specifically binds to CD20 comprising the following six hypervariable regions (HVRs):
(i) an HVR-H1 comprising the amino acid sequence of YSWIN (SEQ ID NO: 1);
(ii) an HVR-H2 comprising the amino acid sequence of RIFPGDGDTDYNGKFKG (SEQ ID NO: 2);
(iii) an HVR-H3 comprising the amino acid sequence of NVFDGYWLVY (SEQ ID NO:3);
(iv) an HVR-L1 comprising the amino acid sequence of RSSKSLLHSNGITYLY (SEQ ID NO: 4);
(v) an HVR-L2 comprising the amino acid sequence of QMSNLVS (SEQ ID NO: 5); and
(vi) an HVR-L3 comprising the amino acid sequence of AQNLELPYT (SEQ ID NO: 6),
and at least one Fab molecule which specifically binds to CD3 comprising the following six HVRs:
(i) an HVR-H1 comprising the amino acid sequence of TYAMN (SEQ ID NO: 9);
(ii) an HVR-H2 comprising the amino acid sequence of RIRSKYNNYATYYADSVKG (SEQ ID NO: 10);
(iii) an HVR-H3 comprising the amino acid sequence of HGNFGNSYVSWFAY (SEQ ID NO: 11);
(iv) an HVR-L1 comprising the amino acid sequence of GSSTGAVTTSNYAN (SEQ ID NO: 12);
(v) an HVR-L2 comprising the amino acid sequence of GTNKRAP (SEQ ID NO: 13); and
(vi) an HVR-L3 comprising the amino acid sequence of ALWYSNLWV (SEQ ID NO: 14).

In one aspect, the invention features a method of treating a subject having a CD20-positive cell proliferative disorder, e.g., a B cell proliferative disorder (e.g., an NHL (e.g., a relapsed and/or refractory NHL, a DLBCL (e.g., a relapsed and/or refractory DLBCL), a FL (e.g., a relapsed and/or refractory FL or a transformed FL), or an MCL (e.g., a relapsed or refractory MCL)), or a CNSL), comprising administering to the subject in a first dosing cycle:
(a) about 2.5 mg (e.g., 2.5 mg±0.01 mg, ±0.02 mg, ±0.03 mg, ±0.05 mg, ±0.1 mg, ±0.2 mg, or ±0.25 mg) as the first dose (C1D1) of a bispecific antibody that binds to CD20 and CD3 and about 10 mg (e.g., 10 mg±0.05 mg, ±0.1 mg, ±0.2 mg, ±0.3 mg, ±0.5 mg, ±0.75 mg, or ±1 mg) as the second dose (C1D2) of the bispecific antibody that binds to CD20 and CD3;
(b) a single dose (C1D1) of about 1000 mg (e.g., 1000 mg±5 mg, ±10 mg, ±20 mg, ±30 mg, ±50 mg, ±75 mg, or ±100 mg) obinutuzumab; and
(c) a single dose (C1D1) of about 5000 mg/m² (e.g., 5000 mg/m²±50 mg/m², ±100 mg/m², ±200 mg/m², ±300 mg/m², ±400 mg/m², or ±500 mg/m²) ifosfamide, a single dose (C1D1) of carboplatin at a dose in mg to target area under the curve (AUC) of 5 mg/mL/min with maximum dose of 750 mg and a first (C1D1), second (C1D2) and third (C1D3) dose of about 100 mg/m² etoposide;
and administering to the subject in a second and third dosing cycle:
(a) a single dose (C2D1 in Cycle 2 and C3D1 in Cycle 3) of about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg) of a bispecific antibody that binds to CD20 and CD3;
(b) a single dose (C2D1 in Cycle 2 and C3D1 in Cycle 3) of about 375 mg/m² (e.g., 375 mg/m²±5 mg/m², ±10 mg/m², ±25 mg/m², or ±37.5 mg/m²) rituximab; and
(c) a single dose (C2D1 in Cycle 2 and C3D1 in Cycle 3) of about 5000 mg/m² (e.g., 5000 mg/m²±50 mg/m², ±100 mg/m², ±200 mg/m², ±300 mg/m², ±400 mg/m², or ±500 mg/m²) ifosfamide, a single dose (C2D1 in Cycle 2 and C3D1 in Cycle 3) of carboplatin at a dose in mg to target area under the curve (AUC) of 5 mg/mL/min with maximum dose of 750 mg and a first (C2D1 in Cycle 2 and C3D1 in Cycle 3), second (C2D2 in Cycle 2 and C3D2 in Cycle 3) and third (C2D3 in Cycle 2 and C3D3 in Cycle 3) dose of about 100 mg/m² etoposide,
wherein the bispecific antibody that binds to CD20 and CD3 comprises at least one Fab molecule which specifically binds to CD20 comprising the following six hypervariable regions (HVRs):
(i) an HVR-H1 comprising the amino acid sequence of YSWIN (SEQ ID NO: 1);
(ii) an HVR-H2 comprising the amino acid sequence of RIFPGDGDTDYNGKFKG (SEQ ID NO: 2);
(iii) an HVR-H3 comprising the amino acid sequence of NVFDGYWLVY (SEQ ID NO:3);
(iv) an HVR-L1 comprising the amino acid sequence of RSSKSLLHSNGITYLY (SEQ ID NO: 4);
(v) an HVR-L2 comprising the amino acid sequence of QMSNLVS (SEQ ID NO: 5); and
(vi) an HVR-L3 comprising the amino acid sequence of AQNLELPYT (SEQ ID NO: 6);
and at least one Fab molecule which specifically binds to CD3 comprising the following six HVRs:
(i) an HVR-H1 comprising the amino acid sequence of TYAMN (SEQ ID NO: 9);
(ii) an HVR-H2 comprising the amino acid sequence of RIRSKYNNYATYYADSVKG (SEQ ID NO: 10;
(iii) an HVR-H3 comprising the amino acid sequence of HGNFGNSYVSWFAY (SEQ ID NO: 11);
(iv) an HVR-L1 comprising the amino acid sequence of GSSTGAVTTSNYAN (SEQ ID NO: 12);
(v) an HVR-L2 comprising the amino acid sequence of GTNKRAP (SEQ ID NO: 13); and
(vi) an HVR-L3 comprising the amino acid sequence of ALWYSNLWV (SEQ ID NO: 14).

In one aspect, the invention features a method of treating a subject having a CD20-positive cell proliferative disorder, e.g., a B cell proliferative disorder (e.g., an NHL (e.g., a relapsed and/or refractory NHL, a DLBCL (e.g., a relapsed and/or refractory DLBCL), a FL (e.g., a relapsed and/or refractory FL or a transformed FL), or an MCL (e.g., a relapsed or refractory MCL)), or a CNSL), comprising administering to the subject:
(a) about 2.5 mg (e.g., 2.5 mg±0.01 mg, ±0.02 mg, ±0.03 mg, ±0.05 mg, ±0.1 mg, ±0.2 mg, or ±0.25 mg) as the first dose (C1D1) of a bispecific antibody that binds to CD20 and CD3 on Day 8, about 10 mg (e.g., 10 mg±0.05 mg, ±0.1 mg, ±0.2 mg, ±0.3 mg, ±0.5 mg, ±0.75 mg, or ±1 mg) as the second dose (C1D2) of the bispecific antibody that binds to CD20 and CD3 on Day 15 of the first dosing cycle and about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg) as the first dose (C2D1) on Day 8 of the second and third dosing cycle; wherein the bispecific antibody that binds to CD20 and CD3 comprises at least one Fab molecule which specifically binds to CD20 comprising the following six hypervariable regions (HVRs):
(i) an HVR-H1 comprising the amino acid sequence of YSWIN (SEQ ID NO: 1);
(ii) an HVR-H2 comprising the amino acid sequence of RIFPGDGDTDYNGKFKG (SEQ ID NO: 2);
(iii) an HVR-H3 comprising the amino acid sequence of NVFDGYWLVY (SEQ ID NO:3);
(iv) an HVR-L1 comprising the amino acid sequence of RSSKSLLHSNGITYLY (SEQ ID NO: 4);
(v) an HVR-L2 comprising the amino acid sequence of QMSNLVS (SEQ ID NO: 5); and
(vi) an HVR-L3 comprising the amino acid sequence of AQNLELPYT (SEQ ID NO: 6),
and at least one Fab molecule which specifically binds to CD3 comprising the following six HVRs:
(i) an HVR-H1 comprising the amino acid sequence of TYAMN (SEQ ID NO: 9);
(ii) an HVR-H2 comprising the amino acid sequence of RIRSKYNNYATYYADSVKG (SEQ ID NO: 10);
(iii) an HVR-H3 comprising the amino acid sequence of HGNFGNSYVSWFAY (SEQ ID NO: 11);
(iv) an HVR-L1 comprising the amino acid sequence of GSSTGAVTTSNYAN (SEQ ID NO: 12);
(v) an HVR-L2 comprising the amino acid sequence of GTNKRAP (SEQ ID NO: 13); and
(vi) an HVR-L3 comprising the amino acid sequence of ALWYSNLWV (SEQ ID NO: 14);
(b) a single dose (C1D1) of about 1000 mg (e.g., 1000 mg±5 mg, ±10 mg, ±20 mg, ±30 mg, ±50 mg, ±75 mg, or ±100 mg) obinutuzumab on Day 1 of the first dosing cycle and a single dose (C2D1) of about 375 mg/m² (e.g., 375 mg/m²±5 mg/m², ±10 mg/m², ±25 mg/m², or ±37.5 mg/m²) rituximab on Day 1 of the second and third dosing cycle; and

(c) a single dose (C1D1) of about 5000 mg/m² (e.g., 5000 mg/m²±50 mg/m², ±100 mg/m², ±200 mg/m², ±300 mg/m², ±400 mg/m², or ±500 mg/m²) ifosfamide on Day 2 of the first and second dosing cycle, a single dose (C1D1) of carboplatin at a dose in mg to target area under the curve (AUC) of 5 mg/mL/min with maximum dose of 750 mg on Day 2 of all three dosing cycles, and a first (C1D1), second (C1D2) and third (C1D3) dose of about 100 mg/m² etoposide on Days 1, 2 and 3 of all three dosing cycles.

In some embodiments, the dosing cycles are 14-day (e.g., 14±3 days) dosing cycles. In some embodiments, the dosing cycles are 21-day (e.g., 21±3 days) dosing cycles. In particular embodiments, the dosing cycles are 21-day dosing cycles.

In some embodiments, the methods featured by the invention further comprises administering to the subject one or more additional therapeutic agents. In some embodiments, the one or more additional therapeutic agents is tocilizumab. In some embodiments, the one or more additional therapeutic agents is a corticosteroid. In some embodiments, the corticosteroid comprises prednisone, prednisolone, methylprednisolone, or dexamethasone. In some embodiments, one or more additional therapeutic agents is an antihistamine. In some embodiments, the antihistamine is diphenhydramine. In some embodiments, the one or more additional therapeutic agents comprises allopurinol and rasburicase. In some embodiments, the one or more additional therapeutic agents is an antipyretic. In some embodiments, the one or more additional therapeutic agents comprises granulocyte colony-stimulating factor (G-CSF). In some embodiments, the one or more additional therapeutic agents is mesna.

In one embodiment, the bispecific antibody is bivalent for CD20 and monovalent for CD3. In one embodiment, the bispecific antibody comprises two Fab molecules which specifically bind to CD20 and one Fab molecule which specifically binds to CD3. In one embodiment the bispecific antibody is a humanized antibody.

In one aspect, the invention features a method of treating a subject having a CD20-positive cell proliferative disorder, e.g., a B cell proliferative disorder (e.g., an NHL (e.g., a relapsed and/or refractory NHL, a DLBCL (e.g., a relapsed and/or refractory DLBCL), a FL (e.g., a relapsed and/or refractory FL or a transformed FL), or an MCL (e.g., a relapsed or refractory MCL)), or a CNSL), comprising administering to the subject an effective amount of:
(a) a bispecific antibody that binds to CD20 and CD3, wherein said bispecific antibody comprises:
at least one Fab molecule which specifically binds to CD20 comprising (a) a VH domain comprising an amino acid sequence of SEQ ID NO: 7 and (b) a VL domain comprising an amino acid sequence of SEQ ID NO: 8 and
at least one Fab molecule which specifically binds to CD3 comprising a VH domain comprising an amino acid sequence of SEQ ID NO: 15 and (b) a VL domain comprising an amino acid sequence of SEQ ID NO: 16;
(b) an anti-CD20 antibody; and
(c) one or more chemotherapeutic agents selected from ifosfamide, carboplatin and/or etoposide in a dosing regimen comprising at least a first dosing cycle and a second dosing cycle.

In one embodiment, the first dosing cycle comprises a first dose (C1D1) of the bispecific antibody and a second dose (C1D2) of the bispecific antibody, wherein the C1D1 of the bispecific antibody is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of the bispecific antibody is about 10 mg (e.g., 10 mg±0.05 mg, ±0.1 mg, ±0.2 mg, ±0.3 mg, ±0.5 mg, ±0.75 mg, or ±1 mg); and the second dosing cycle comprises a single dose (C2D1) of the bispecific antibody, wherein the C2D1 of the bispecific antibody is about 10 mg (e.g., 10 mg±0.05 mg, ±0.1 mg, ±0.2 mg, ±0.3 mg, ±0.5 mg, ±0.75 mg, or ±1 mg), about 16 mg (e.g., 16 mg±0.05 mg, ±0.1 mg, ±0.2 mg, ±0.3 mg, ±0.5 mg, ±0.75 mg, ±1 mg, ±1.5 mg, or ±1.6 mg), or about 30 mg (e.g., 30 mg ±0.1 mg, ±0.2 mg, ±0.3 mg, ±0.5 mg, ±0.75 mg, ±1 mg, ±1.5 mg, ±2 mg, or ±3 mg). In particular embodiments, the C1D1 of the bispecific antibody is about 2.5 mg (e.g., 2.5 mg±0.01 mg, ±0.02 mg, ±0.03 mg, ±0.05 mg, ±0.1 mg, ±0.2 mg, or ±0.25 mg) and the C1D2 of the bispecific antibody is about 10 mg (e.g., 10 mg±0.05 mg, ±0.1 mg, ±0.2 mg, ±0.3 mg, ±0.5 mg, ±0.75 mg, or ±1 mg). In particular embodiments, the C2D1 is about 10 mg (e.g., 10 mg±0.05 mg, ±0.1 mg, ±0.2 mg, ±0.3 mg, ±0.5 mg, ±0.75 mg, or ±1 mg). In particular embodiments, the C2D1 of the bispecific antibody is about 16 mg (e.g., 16 mg±0.05 mg, ±0.1 mg, ±0.2 mg, ±0.3 mg, ±0.5 mg, ±0.75 mg, ±1 mg, ±1.5 mg, or ±1.6 mg). In particular embodiments, the C2D1 of the bispecific antibody is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one embodiment, the first dosing cycle comprises a first dose (C1D1) of the bispecific antibody and a second dose (C1D2) of the bispecific antibody, wherein the C1D1 of the bispecific antibody is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of the bispecific antibody is about 10 mg (e.g., 10 mg±0.05 mg, ±0.1 mg, ±0.2 mg, ±0.3 mg, ±0.5 mg, ±0.75 mg, or ±1 mg); and the second dosing cycle comprises a single dose (C2D1) of the bispecific antibody, wherein the C2D1 of the bispecific antibody is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one embodiment, the first dose (C1D1) of the bispecific antibody and the second dose (C1D2) of the bispecific antibody are administered to the subject on or about Days 8 (±1 day) and 15 (±1 day), respectively, of the first dosing cycle.

In one embodiment, the first dose (C1D1) of the bispecific antibody and the second dose (C1D2) of the bispecific antibody are administered to the subject on Days 8 and 15, respectively, of the first dosing cycle. In some embodiments, the C2D1 of the bispecific antibody is administered to the subject on or about Day 8 (±1 day) of the second dosing cycle. In some embodiments, the C2D1 of the bispecific antibody is administered to the subject on or about Day 8 of the second dosing cycle.

In one embodiment, the anti-CD20 antibody is obinutuzumab and/or rituximab. In one embodiment, the first dosing cycle comprises a single dose (C1D1) of obinutuzumab; and the second dosing cycle comprises a single dose (C2D1) of rituximab.

In one embodiment, the single dose C1D1 of the obinutuzumab is about 1000 mg (e.g., 1000 mg ±5 mg, ±10 mg, ±20 mg, ±30 mg, ±50 mg, ±75 mg, or ±100 mg) and the single dose of rituximab is about 375 mg/m² (e.g., 375 mg/m²±5 mg/m², ±10 mg/m², ±25 mg/m², or ±37.5 mg/m²).

In one embodiment, the anti-CD20 antibody is administered in a dosing regimen comprising at least a first dosing cycle and a second dosing cycle, wherein the first dosing cycle comprises a single dose (C1D1) of obinutuzumab on Day 1; and the second dosing cycle comprises a single dose (C2D1) of rituximab on Day 1.

In one aspect, the invention features a method of treating a subject having a CD20-positive cell proliferative disorder, e.g., a B cell proliferative disorder (e.g., an NHL (e.g., a relapsed and/or refractory NHL, a DLBCL (e.g., a relapsed and/or refractory DLBCL), a FL (e.g., a relapsed and/or refractory FL or a transformed FL), or an MCL (e.g., a relapsed or refractory MCL)), or a CNSL), comprising administering to the subject in a first dosing cycle:
(a) about 2.5 mg (e.g., 2.5 mg±0.01 mg, ±0.02 mg, ±0.03 mg, ±0.05 mg, ±0.1 mg, ±0.2 mg, or ±0.25 mg) as the first dose (C1D1) of a bispecific antibody that binds to CD20 and CD3 and about 10 mg (e.g., 10 mg±0.05 mg, ±0.1 mg, ±0.2 mg, ±0.3 mg, ±0.5 mg, ±0.75 mg, or ±1 mg) as the second dose (C1D2) of the bispecific antibody that binds to CD20 and CD3;
(b) a single dose (C1D1) of obinutuzumab; and
(c) one or more chemotherapeutic agents selected from ifosfamide, carboplatin and/or etoposide;
and administering to the subject in a second dosing cycle:
(a) a single dose (C2D1) of about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg) of a bispecific antibody that binds to CD20 and CD3
(b) a single dose (C2D1) of rituximab; and
(c) one or more chemotherapeutic agents selected from ifosfamide, carboplatin and/or etoposide,
wherein said bispecific antibody comprises:
at least one Fab molecule which specifically binds to CD20 comprising (a) a VH domain comprising an amino acid sequence of SEQ ID NO: 7 and (b) a VL domain comprising an amino acid sequence of SEQ ID NO: 8 and
at least one Fab molecule which specifically binds to CD3 comprising a VH domain comprising an amino acid sequence of SEQ ID NO: 15 and (b) a VL domain comprising an amino acid sequence of SEQ ID NO: 16.

In one embodiment, the single dose C1D1 of the obinutuzumab is about 1000 mg (e.g., 1000 mg ±5 mg, ±10 mg, ±20 mg, ±30 mg, ±50 mg, ±75 mg, or ±100 mg) and the single dose of rituximab is about 375 mg/m² (e.g., 375 mg/m²±5 mg/m², ±10 mg/m², ±25 mg/m², or ±37.5 mg/m²).

In one embodiment, the anti-CD20 antibody is administered in a dosing regimen comprising at least a first dosing cycle and a second dosing cycle, wherein the first dosing cycle comprises a single dose (C1D1) of obinutuzumab on Day 1; and the second dosing cycle comprises a single dose (C2D1) of rituximab on Day 1.

In one embodiment, the first dose (C1D1) of the bispecific antibody and the second dose (C1D2) of the bispecific antibody are administered to the subject on or about Days 8 (±1 day) and 15 (±1 day), respectively, of the first dosing cycle.

In one embodiment, the first dose (C1D1) of the bispecific antibody and the second dose (C1D2) of the bispecific antibody are administered to the subject on Days 8 and 15, respectively, of the first dosing cycle. In some embodiments, the C2D1 of the bispecific antibody is administered to the subject on or about Day 8 (±1 day) of the second dosing cycle. In some embodiments, the C2D1 of the bispecific antibody is administered to the subject on or about Day 8 of the second dosing cycle.

In one embodiment of the methods provided above, step c) comprises all three chemotherapeutic agents.

In one embodiment, the first dosing cycle comprises a single dose (C1D1) of ifosfamide, a single dose (C1D1) of carboplatin and a first (C1D1), second (C1D2) and third (C1D3) dose of etoposide; and the second cycle each comprises a single dose (C2D1) of ifosfamide, a single dose (C2D1) of carboplatin and a first (C2D1), second (C2D2) and third (C2D3) dose of etoposide.

In one aspect, the invention features a method of treating a subject having a CD20-positive cell proliferative disorder, e.g., a B cell proliferative disorder (e.g., an NHL (e.g., a relapsed and/or refractory NHL, a DLBCL (e.g., a relapsed and/or refractory DLBCL), a FL (e.g., a relapsed and/or refractory FL or a transformed FL), or an MCL (e.g., a relapsed or refractory MCL)), or a CNSL), comprising administering to the subject in a first dosing cycle:
(a) about 2.5 mg (e.g., 2.5 mg±0.01 mg, ±0.02 mg, ±0.03 mg, ±0.05 mg, ±0.1 mg, ±0.2 mg, or ±0.25 mg) as the first dose (C1D1) of a bispecific antibody that binds to CD20 and CD3 and about 10 mg (e.g., 10 mg±0.05 mg, ±0.1 mg, ±0.2 mg, ±0.3 mg, ±0.5 mg, ±0.75 mg, or ±1 mg) as the second dose (C1D2) of the bispecific antibody that binds to CD20 and CD3;
(b) a single dose (C1D1) of obinutuzumab; and
(c) a single dose (C1D1) of ifosfamide, a single dose (C1D1) of carboplatin and a first (C1D1), second (C1D2) and third (C1D3) dose of etoposide;
and administering to the subject in a second dosing cycle:
(a) a single dose (C2D1) of about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg) of a bispecific antibody that binds to CD20 and CD3;
(b) a single dose (C2D1) of rituximab; and
(c) a single dose (C2D1) of ifosfamide, a single dose (C2D1) of carboplatin and a first (C2D1), second (C2D2) and third (C2D3) dose of etoposide,
wherein said bispecific antibody comprises:
at least one Fab molecule which specifically binds to CD20 comprising (a) a VH domain comprising an amino acid sequence of SEQ ID NO: 7 and (b) a VL domain comprising an amino acid sequence of SEQ ID NO: 8 and
at least one Fab molecule which specifically binds to CD3 comprising a VH domain comprising an amino acid sequence of SEQ ID NO: 15 and (b) a VL domain comprising an amino acid sequence of SEQ ID NO: 16.

In one embodiment, the single dose C1D1 of the obinutuzumab is about 1000 mg (e.g., 1000 mg ±5 mg, ±10 mg, ±20 mg, ±30 mg, ±50 mg, ±75 mg, or ±100 mg) and the single dose of rituximab is about 375 mg/m² (e.g., 375 mg/m²±5 mg/m², ±10 mg/m², ±25 mg/m², or ±37.5 mg/m²).

In one embodiment, the anti-CD20 antibody is administered in a dosing regimen comprising at least a first dosing cycle and a second dosing cycle, wherein the first dosing cycle comprises a single dose (C1D1) of obinutuzumab on Day 1; and the second dosing cycle comprises a single dose (C2D1) of rituximab on Day 1.

In one embodiment, the first dose (C1D1) of the bispecific antibody and the second dose (C1D2) of the bispecific antibody are administered to the subject on or about Days 8 (±1 day) and 15 (±1 day), respectively, of the first dosing cycle.

In one embodiment, the first dose (C1D1) of the bispecific antibody and the second dose (C1D2) of the bispecific antibody are administered to the subject on Days 8 and 15, respectively, of the first dosing cycle. In some embodiments, the C2D1 of the bispecific antibody is administered to the subject on or about Day 8 (±1 day) of the second dosing cycle. In some embodiments, the C2D1 of the bispecific antibody is administered to the subject on or about Day 8 of the second dosing cycle.

In one embodiment of the methods provided above ifosfamide is administered at a dose of 5000 mg/m², 4000 mg/m² or 1666 mg/m². In one embodiment of the methods provided above ifosfamide is administered at a dose of 5000 mg/m². In one embodiment of the methods provided above, carboplatin is administered at a dose in mg to target area under the curve (AUC) of 5 mg/mL/min with maximum dose of 750 mg. In one embodiment of the methods provided above etoposide is administered at a dose of 100 mg/m² or 75 mg/m². In one embodiment of the methods provided above etoposide is administered at a dose of 100 mg/m².

In one embodiment of the methods provided above ifosfamide is administered at a dose of 5000 mg/m², 4000 mg/m² or 1666 mg/m², carboplatin is administered at a dose in mg to target area under the curve (AUC) of 5 mg/mL/min with maximum dose of 750 mg and etoposide is administered at a dose of 100 mg/m² or 75 mg/m².

In one embodiment of the methods provided above ifosfamide is administered at a dose of 5000 mg/m², carboplatin is administered at a dose in mg to target area under the curve (AUC) of 5 mg/mL/min with maximum dose of 750 mg and etoposide is administered at a dose of 100 mg/m².

In one embodiment, ifosfamide and carboplatin are administered on Day 2 of the first and second dosing cycle and etoposide is administered on each of Days 1, 2 and 3 of the first and second dosing cycle.

In one aspect, the invention features a method of treating a subject having a CD20-positive cell proliferative disorder, e.g., a B cell proliferative disorder (e.g., an NHL (e.g., a relapsed and/or refractory NHL, a DLBCL (e.g., a relapsed and/or refractory DLBCL), a FL (e.g., a relapsed and/or refractory FL or a transformed FL), or an MCL (e.g., a relapsed or refractory MCL)), or a CNSL), comprising administering to the subject in a first dosing cycle:
(a) about 2.5 mg (e.g., 2.5 mg±0.01 mg, ±0.02 mg, ±0.03 mg, ±0.05 mg, ±0.1 mg, ±0.2 mg, or ±0.25 mg) as the first dose (C1D1) of a bispecific antibody that binds to CD20 and CD3 and about 10 mg (e.g., 10 mg±0.05 mg, ±0.1 mg, ±0.2 mg, ±0.3 mg, ±0.5 mg, ±0.75 mg, or ±1 mg) as the second dose (C1D2) of the bispecific antibody that binds to CD20 and CD3; and
(b) a single dose (C1D1) of about 1000 mg (e.g., 1000 mg±5 mg, ±10 mg, ±20 mg, ±30 mg, ±50 mg, ±75 mg, or ±100 mg) obinutuzumab; and
(c) a single dose (C1D1) of about 5000 mg/m² (e.g., 5000 mg/m²±50 mg/m², ±100 mg/m², ±200 mg/m², ±300 mg/m², ±400 mg/m², or ±500 mg/m²) ifosfamide, a single dose (C1D1) of carboplatin at a dose in mg to target area under the curve (AUC) of 5 mg/mL/min with maximum dose of 750 mg and a first (C1D1), second (C1D2) and third (C1D3) dose of about 100 mg/m² etoposide;
and administering to the subject in a second dosing cycle:
(a) a single dose (C2D1) of about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg) of a bispecific antibody that binds to CD20 and CD3;
(b) a single dose (C2D1) of about 375 mg/m² (e.g., 375 mg/m²±5 mg/m², ±10 mg/m², ±25 mg/m², or ±37.5 mg/m²) rituximab; and
(c) a single dose (C1D1) of about 5000 mg/m² (e.g., 5000 mg/m²±50 mg/m², ±100 mg/m², ±200 mg/m², ±300 mg/m², ±400 mg/m², or ±500 mg/m²) ifosfamide, a single dose (C1D1) of carboplatin at a dose in mg to target area under the curve (AUC) of 5 mg/mL/min with maximum dose of 750 mg and a first (C1D1), second (C1D2) and third (C1D3) dose of about 100 mg/m² etoposide;
wherein said bispecific antibody comprises:
at least one Fab molecule which specifically binds to CD20 comprising (a) a VH domain comprising an amino acid sequence of SEQ ID NO: 7 and (b) a VL domain comprising an amino acid sequence of SEQ ID NO: 8 and
at least one Fab molecule which specifically binds to CD3 comprising a VH domain comprising an amino acid sequence of SEQ ID NO: 15 and (b) a VL domain comprising an amino acid sequence of SEQ ID NO: 16.

In one aspect, the invention features a method of treating a subject having a CD20-positive cell proliferative disorder, e.g., a B cell proliferative disorder (e.g., an NHL (e.g., a relapsed and/or refractory NHL, a DLBCL (e.g., a relapsed and/or refractory DLBCL), a FL (e.g., a relapsed and/or refractory FL or a transformed FL), or an MCL (e.g., a relapsed or refractory MCL)), or a CNSL), comprising administering to the subject:
(a) about 2.5 mg (e.g., 2.5 mg±0.01 mg, ±0.02 mg, ±0.03 mg, ±0.05 mg, ±0.1 mg, ±0.2 mg, or ±0.25 mg) as the first dose (C1D1) of a bispecific antibody that binds to CD20 and CD3 on Day 8, about 10 mg (e.g., 10 mg±0.05 mg, ±0.1 mg, ±0.2 mg, ±0.3 mg, ±0.5 mg, ±0.75 mg, or ±1 mg) as the second dose (C1D2) of the bispecific antibody that binds to CD20 and CD3 on Day 15 of the first dosing cycle and about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg) as the first dose (C2D1) on Day 8 of the second dosing cycle;
(b) a single dose (C1D1) of about 1000 mg (e.g., 1000 mg±5 mg, ±10 mg, ±20 mg, ±30 mg, ±50 mg, ±75 mg, or ±100 mg) obinutuzumab on Day 1 of the first dosing cycle and a single dose (C2D1) of about 375 mg/m² (e.g., 375 mg/m²±5 mg/m², ±10 mg/m², ±25 mg/m², or ±37.5 mg/m²) rituximab on Day 1 of the second dosing cycle; and
(c) a single dose (C1D1) of about 5000 mg/m² (e.g., 5000 mg/m²±50 mg/m², ±100 mg/m², ±200 mg/m², ±300 mg/m², ±400 mg/m², or ±500 mg/m²) ifosfamide on Day 2 of the first and second dosing cycle, a single dose (C1D1) of carboplatin at a dose in mg to target area under the curve (AUC) of 5 mg/mL/min with maximum dose of 750 mg on Day 2 of the first and second dosing cycle, and a first (C1D1), second (C1D2) and third (C1D3) dose of about 100 mg/m² etoposide on Days 1, 2 and 3 of the first and second dosing cycle;
wherein said bispecific antibody comprises:
at least one Fab molecule which specifically binds to CD20 comprising (a) a VH domain comprising an amino acid sequence of SEQ ID NO: 7 and (b) a VL domain comprising an amino acid sequence of SEQ ID NO: 8 and
at least one Fab molecule which specifically binds to CD3 comprising a VH domain comprising an amino acid sequence of SEQ ID NO: 15 and (b) a VL domain comprising an amino acid sequence of SEQ ID NO: 16.

In some embodiments, the first and second dosing cycles are 14-day (e.g., 14±3 days) dosing cycles. In some embodiments, the first and second dosing cycles are 21-day (e.g., 21±3 days) dosing cycles. In particular embodiments, the first and second dosing cycles are 21-day dosing cycles.

In some embodiments, the dosing regimen comprises one or more additional dosing cycles. In some embodiments, the dosing regimen comprises three dosing cycles in total. In some embodiments, the additional dosing cycles are 14-day (e.g., 14±3 days) dosing cycles. In some embodiments, the additional dosing cycles are 21-day (e.g., 21±3 days) dosing cycles. In one embodiment, the method provided herein comprises three 21-day dosing cycles.

In one embodiment, the one or more additional dosing cycles comprise
(a) an additional single dose of the bispecific antibody that binds to CD20 and CD3, wherein said bispecific antibody comprises:
at least one Fab molecule which specifically binds to CD20 comprising (a) a VH domain comprising an amino acid sequence of SEQ ID NO: 7 and (b) a VL domain comprising an amino acid sequence of SEQ ID NO: 8 and
at least one Fab molecule which specifically binds to CD3 comprising a VH domain comprising an amino acid sequence of SEQ ID NO: 15 and (b) a VL domain comprising an amino acid sequence of SEQ ID NO: 16;
(b) an additional single dose of the anti-CD20 antibody; and
(c) an additional single dose of ifosfamide, an additional single dose of carboplatin and an additional first, second and third dose of etoposide.

In one embodiment, the additional single dose of the bispecific antibody is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one embodiment, the anti-CD20 antibody is rituximab. In one embodiment, the additional single dose of rituximab is about 375 mg/m² (e.g., 375 mg/m²±5 mg/m², ±10 mg/m², ±25 mg/m², or ±37.5 mg/m²). In one embodiment, the additional single dose of rituximab is administered on Day 1 of the additional dosing cycle.

In one embodiment, the additional dose of ifosfamide is about 5000 mg/m² (e.g., 5000 mg/m²±50 mg/m², ±100 mg/m², ±200 mg/m², ±300 mg/m², ±400 mg/m², or ±500 mg/m²), about 4000 mg/m² (e.g., 4000 mg/m²±40 mg/m², ±50 mg/m², ±100 mg/m², ±200 mg/m², ±300 mg/m², or ±400 mg/m²), or about 1666 mg/m² (e.g., 1666 mg/m²±25 mg/m², ±50 mg/m², ±100 mg/m², or ±166.6 mg/m²), the additional single dose of carboplatin is in mg to target area under the curve (AUC) of about 5 mg/mL/min (e.g., 5 mg/mL/min±0.05 mg/mL/min, ±0.1 mg/mL/min, ±0.25 mg/mL/min, or ±0.5 mg/mL/min) with maximum dose of about 750 mg (e.g., 750 mg±10 mg, ±25 mg, ±50 mg, or ±75 mg) and the additional first, second and third dose of etoposide is 100 mg/m² (e.g., 100 mg/m²±1 mg/m², ±2.5 mg/m², ±5 mg/m², or ±10 mg/m²) or 75 mg/m² (e.g., 0.5 mg/m²±1 mg/m², ±2.5 mg/m², ±5 mg/m², or ±7.5 mg/m²). In one embodiment, the additional single dose of ifosfamide is 5000 mg/m², 4000 mg/m² or 1666 mg/m², the additional single dose of carboplatin is in mg to target area under the curve (AUC) of 5 mg/mL/min with maximum dose of 750 mg and the additional first, second and third dose of etoposide is 100 mg/m² or 75 mg/m². In one embodiment, ifosfamide is administered at a dose of 5000 mg/m², carboplatin is administered at a dose in mg to target area under the curve (AUC) of 5 mg/mL/min with maximum dose of 750 mg and etoposide is administered at a dose of 100 mg/m².

In one embodiment, ifosfamide and carboplatin are administered on Day 2 of the additional dosing cycle and etoposide is administered on each of Days 1, 2 and 3 of the additional dosing cycle.

In one aspect, the invention features a method of treating a subject having a CD20-positive cell proliferative disorder, e.g., a B cell proliferative disorder (e.g., an NHL (e.g., a relapsed and/or refractory NHL, a DLBCL (e.g., a relapsed and/or refractory DLBCL), a FL (e.g., a relapsed and/or refractory FL or a transformed FL), or an MCL (e.g., a relapsed or refractory MCL)), or a CNSL), comprising administering to the subject in a first dosing cycle:
(a) about 2.5 mg (e.g., 2.5 mg±0.01 mg, ±0.02 mg, ±0.03 mg, ±0.05 mg, ±0.1 mg, ±0.2 mg, or ±0.25 mg) as the first dose (C1D1) of a bispecific antibody that binds to CD20 and CD3 and about 10 mg (e.g., 10 mg±0.05 mg, ±0.1 mg, ±0.2 mg, ±0.3 mg, ±0.5 mg, ±0.75 mg, or ±1 mg) as the second dose (C1D2) of the bispecific antibody that binds to CD20 and CD3;
(b) a single dose (C1D1) of obinutuzumab; and
(c) a single dose (C1D1) of ifosfamide, a single dose (C1D1) of carboplatin and a first (C1D1), second (C1D2) and third (C1D3) dose of etoposide;
and administering to the subject in a second and third dosing cycle:
(a) a single dose (C2D1 in Cycle 2 and C3D1 in Cycle 3) of about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg) of a bispecific antibody that binds to CD20 and CD3;
(b) a single dose (C2D1 in Cycle 2 and C3D1 in Cycle 3) of rituximab; and
(c) a single dose (C2D1 in Cycle 2 and C3D1 in Cycle 3) of ifosfamide, a single dose (C2D1 in Cycle 2 and C3D1 in Cycle 3) of carboplatin and a first (C2D1 in Cycle 2 and C3D2 in Cycle 3), second (C2D2 in Cycle 2 and C3D2 in Cycle 3) and third (C2D3 in Cycle 2 and C3D3 in Cycle 3) dose of etoposide;
wherein said bispecific antibody comprises:
at least one Fab molecule which specifically binds to CD20 comprising (a) a VH domain comprising an amino acid sequence of SEQ ID NO: 7 and (b) a VL domain comprising an amino acid sequence of SEQ ID NO: 8 and
at least one Fab molecule which specifically binds to CD3 comprising a VH domain comprising an amino acid sequence of SEQ ID NO: 15 and (b) a VL domain comprising an amino acid sequence of SEQ ID NO: 16.

In one aspect, the invention features a method of treating a subject having a CD20-positive cell proliferative disorder, e.g., a B cell proliferative disorder (e.g., an NHL (e.g., a relapsed and/or refractory NHL, a DLBCL (e.g., a relapsed and/or refractory DLBCL), a FL (e.g., a relapsed and/or refractory FL or a transformed FL), or an MCL (e.g., a relapsed or refractory MCL)), or a CNSL), comprising administering to the subject in a first dosing cycle:
(a) about 2.5 mg (e.g., 2.5 mg±0.01 mg, ±0.02 mg, ±0.03 mg, ±0.05 mg, ±0.1 mg, ±0.2 mg, or ±0.25 mg) as the first dose (C1D1) of a bispecific antibody that binds to CD20 and CD3 and about 10 mg (e.g., 10 mg±0.05 mg, ±0.1 mg, ±0.2 mg, ±0.3 mg, ±0.5 mg, ±0.75 mg, or ±1 mg) as the second dose (C1D2) of the bispecific antibody that binds to CD20 and CD3;
(b) a single dose (C1D1) of about 1000 mg (e.g., 1000 mg±5 mg, ±10 mg, ±20 mg, ±30 mg, ±50 mg, ±75 mg, or ±100 mg) obinutuzumab; and
(c) a single dose (C1D1) of about 5000 mg/m² (e.g., 5000 mg/m²±50 mg/m², ±100 mg/m², ±200 mg/m², ±300 mg/m², ±400 mg/m², or ±500 mg/m²) ifosfamide, a single dose (C1D1) of carboplatin at a dose in mg to target area under the curve (AUC) of 5 mg/mL/min with maximum dose of 750 mg and a first (C1D1), second (C1D2) and third (C1D3) dose of about 100 mg/m² etoposide;
and administering to the subject in a second and third dosing cycle:
(a) a single dose (C2D1 in Cycle 2 and C3D1 in Cycle 3) of about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg) of a bispecific antibody that binds to CD20 and CD3;
(b) a single dose (C2D1 in Cycle 2 and C3D1 in Cycle 3) of about 375 mg/m² (e.g., 375 mg/m²±5 mg/m², ±10 mg/m², ±25 mg/m², or ±37.5 mg/m²) rituximab; and
(c) a single dose (C2D1 in Cycle 2 and C3D1 in Cycle 3) of about 5000 mg/m² (e.g., 5000 mg/m²±50 mg/m², ±100 mg/m², ±200 mg/m², ±300 mg/m², ±400 mg/m², or ±500 mg/m²) ifosfamide, a single dose (C2D1 in Cycle 2 and C3D1 in Cycle 3) of carboplatin at a dose in mg to target area under the curve (AUC) of 5 mg/mL/min with maximum dose of 750 mg and a first (C2D1 in Cycle 2 and C3D1 in Cycle 3), second (C2D2 in Cycle 2 and C3D2 in Cycle 3) and third (C2D3 in Cycle 2 and C3D3 in Cycle 3) dose of about 100 mg/m² etoposide,
wherein said bispecific antibody comprises:
at least one Fab molecule which specifically binds to CD20 comprising (a) a VH domain comprising an amino acid sequence of SEQ ID NO: 7 and (b) a VL domain comprising an amino acid sequence of SEQ ID NO: 8 and
at least one Fab molecule which specifically binds to CD3 comprising a VH domain comprising an amino acid sequence of SEQ ID NO: 15 and (b) a VL domain comprising an amino acid sequence of SEQ ID NO: 16.

In one aspect, the invention features a method of treating a subject having a CD20-positive cell proliferative disorder, e.g., a B cell proliferative disorder (e.g., an NHL (e.g., a relapsed and/or refractory NHL, a DLBCL (e.g., a relapsed and/or refractory DLBCL), a FL (e.g., a relapsed and/or refractory FL or a transformed FL), or an MCL (e.g., a relapsed or refractory MCL)), or a CNSL), comprising administering to the subject:
(a) about 2.5 mg (e.g., 2.5 mg±0.01 mg, ±0.02 mg, ±0.03 mg, ±0.05 mg, ±0.1 mg, ±0.2 mg, or ±0.25 mg) as the first dose (C1D1) of a bispecific antibody that binds to CD20 and CD3 on Day 8, about 10 mg (e.g., 10 mg±0.05 mg, ±0.1 mg, ±0.2 mg, ±0.3 mg, ±0.5 mg, ±0.75 mg, or ±1 mg) as the second dose (C1D2) of the bispecific antibody that binds to CD20 and CD3 on Day 15 of the first dosing cycle and about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg) as the first dose (C2D1) on Day 8 of the second and third dosing cycle, wherein said bispecific antibody comprises:
at least one Fab molecule which specifically binds to CD20 comprising (a) a VH domain comprising an amino acid sequence of SEQ ID NO: 7 and (b) a VL domain comprising an amino acid sequence of SEQ ID NO: 8 and
at least one Fab molecule which specifically binds to CD3 comprising a VH domain comprising an amino acid sequence of SEQ ID NO: 15 and (b) a VL domain comprising an amino acid sequence of SEQ ID NO: 16;
(b) a single dose (C1D1) of about 1000 mg (e.g., 1000 mg±5 mg, ±10 mg, ±20 mg, ±30 mg, ±50 mg, ±75 mg, or ±100 mg) obinutuzumab on Day 1 of the first dosing cycle and a single dose (C2D1) of about 375 mg/m² (e.g., 375 mg/m²±5 mg/m², ±10 mg/m², ±25 mg/m², or ±37.5 mg/m²) rituximab on Day 1 of the second and third dosing cycle; and
(c) a single dose (C1D1) of about 5000 mg/m² (e.g., 5000 mg/m²±50 mg/m², ±100 mg/m², ±200 mg/m², ±300 mg/m², ±400 mg/m², or ±500 mg/m²) ifosfamide on Day 2 of the first and second dosing cycle, a single dose (C1D1) of carboplatin at a dose in mg to target area under the curve (AUC) of 5 mg/mL/min with maximum dose of 750 mg on Day 2 of all three dosing cycles, and a first (C1D1), second (C1D2) and third (C1D3) dose of about 100 mg/m² etoposide on Days 1, 2 and 3 of all three dosing cycles.

In some embodiments, the dosing cycles are 14-day (e.g., 14±3 days) dosing cycles. In some embodiments, the dosing cycles are 21-day (e.g., 21±3 days) dosing cycles. In particular embodiments, the dosing cycles are 21-day dosing cycles.

In some embodiments, the methods featured by the invention further comprises administering to the subject one or more additional therapeutic agents. In some embodiments, the one or more additional therapeutic agents is tocilizumab. In some embodiments, the one or more additional therapeutic agents is a corticosteroid. In some embodiments, the corticosteroid comprises prednisone, prednisolone, methylprednisolone, or dexamethasone. In some embodiments, one or more additional therapeutic agents is an antihistamine. In some embodiments, the antihistamine is diphenhydramine. In some embodiments, the one or more additional therapeutic agents comprises allopurinol and rasburicase. In some embodiments, the one or more additional therapeutic agents is an antipyretic. In some embodiments, the one or more additional therapeutic agents comprises granulocyte colony-stimulating factor (G-CSF). In some embodiments, the one or more additional therapeutic agents is mesna.

In one embodiment, the bispecific antibody is bivalent for CD20 and monovalent for CD3. In one embodiment, the bispecific antibody comprises two Fab molecules which specifically bind to CD20 and one Fab molecule which specifically binds to CD3. In one embodiment the bispecific antibody is a humanized antibody. In one embodiment, the bispecific antibody is glofitamab.

In one aspect, the invention features a method of treating a subject having a CD20-positive cell proliferative disorder, e.g., a B cell proliferative disorder (e.g., an NHL (e.g., a relapsed and/or refractory NHL, a DLBCL (e.g., a relapsed and/or refractory DLBCL), a FL (e.g., a relapsed and/or refractory FL or a transformed FL), or an MCL (e.g., a relapsed or refractory MCL)), or a CNSL), comprising administering to the subject an effective amount of:
(a) glofitamab;
(b) an anti-CD20 antibody; and
(c) one or more chemotherapeutic agents selected from ifosfamide, carboplatin and/or etoposide in a dosing regimen comprising at least a first dosing cycle and a second dosing cycle.

In one embodiment, the first dosing cycle comprises a first dose (C1D1) of glofitamab and a second dose (C1D2) of glofitamab, wherein the C1D1 of glofitamab is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 10 mg (e.g., 10 mg±0.05 mg, ±0.1 mg, ±0.2 mg, ±0.3 mg, ±0.5 mg, ±0.75 mg, or ±1 mg); and the second dosing cycle comprises a single dose (C2D1) of glofitamab, wherein the C2D1 of glofitamab is about 10 mg (e.g., 10 mg±0.05 mg, ±0.1 mg, ±0.2 mg, ±0.3 mg, ±0.5 mg, ±0.75 mg, or ±1 mg), about 16 mg (e.g., 16 mg±0.05 mg, ±0.1 mg, ±0.2 mg, ±0.3 mg, ±0.5 mg, ±0.75 mg, ±1 mg, ±1.5 mg, or ±1.6 mg), or about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg). In particular embodiments, the C1D1 of glofitamab is about 2.5 mg (e.g., 2.5 mg±0.01 mg, ±0.02 mg, ±0.03 mg, ±0.05 mg, ±0.1 mg, ±0.2 mg, or ±0.25 mg) and the C1D2 of glofitamab is about 10 mg (e.g., 10 mg±0.05 mg, ±0.1 mg, ±0.2 mg, ±0.3 mg, ±0.5 mg, ±0.75 mg, or ±1 mg). In particular embodiments, the C2D1 is about 10 mg (e.g., 10 mg±0.05 mg, ±0.1 mg, ±0.2 mg, ±0.3 mg, ±0.5 mg, ±0.75 mg, or ±1 mg). In particular embodiments, the C2D1 of glofitamab is about 16 mg (e.g., 16 mg±0.05 mg, ±0.1 mg, ±0.2 mg, ±0.3 mg, ±0.5 mg, ±0.75 mg, ±1 mg, ±1.5 mg, or ±1.6 mg). In particular embodiments, the C2D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one embodiment, the first dosing cycle comprises a first dose (C1D1) of glofitamab and a second dose (C1D2) of glofitamab, wherein the C1D1 of glofitamab is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of the glofitamab is about 10 mg (e.g., 10 mg±0.05 mg, ±0.1 mg, ±0.2 mg, ±0.3 mg, ±0.5 mg, ±0.75 mg, or ±1 mg); and the second dosing cycle comprises a single dose (C2D1) of glofitamab, wherein the C2D1 of the bispecific antibody is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one embodiment, the first dose (C1D1) of glofitamab and the second dose (C1D2) of glofitamab are administered to the subject on or about Days 8 (±1 day) and 15 (±1 day), respectively, of the first dosing cycle.

In one embodiment, the first dose (C1D1) of glofitamab and the second dose (C1D2) of glofitamab are administered to the subject on Days 8 and 15, respectively, of the first dosing cycle. In some embodiments, the C2D1 of glofitamab is administered to the subject on or about Day 8 (±1 day) of the second dosing cycle. In some embodiments, the C2D1 of glofitamab is administered to the subject on or about Day 8 of the second dosing cycle.

In one embodiment, the anti-CD20 antibody is obinutuzumab and/or rituximab. In one embodiment, the first dosing cycle comprises a single dose (C1D1) of obinutuzumab; and the second dosing cycle comprises a single dose (C2D1) of rituximab.

In one embodiment, the single dose C1D1 of the obinutuzumab is about 1000 mg (e.g., 1000 mg ±5 mg, ±10 mg, ±20 mg, ±30 mg, ±50 mg, ±75 mg, or ±100 mg) and the single dose of rituximab is about 375 mg/m² (e.g., 375 mg/m²±5 mg/m², ±10 mg/m², ±25 mg/m², or ±37.5 mg/m²).

In one embodiment, the anti-CD20 antibody is administered in a dosing regimen comprising at least a first dosing cycle and a second dosing cycle, wherein the first dosing cycle comprises a single dose (C1D1) of obinutuzumab on Day 1; and the second dosing cycle comprises a single dose (C2D1) of rituximab on Day 1.

In one aspect, the invention features a method of treating a subject having a CD20-positive cell proliferative disorder, e.g., a B cell proliferative disorder (e.g., an NHL (e.g., a relapsed and/or refractory NHL, a DLBCL (e.g., a relapsed and/or refractory DLBCL), a FL (e.g., a relapsed and/or refractory FL or a transformed FL), or an MCL (e.g., a relapsed or refractory MCL)), or a CNSL), comprising administering to the subject in a first dosing cycle:
(a) about 2.5 mg (e.g., 2.5 mg±0.01 mg, ±0.02 mg, ±0.03 mg, ±0.05 mg, ±0.1 mg, ±0.2 mg, or ±0.25 mg) as the first dose (C1D1) of glofitamab and about 10 mg (e.g., 10 mg±0.05 mg, ±0.1 mg, ±0.2 mg, ±0.3 mg, ±0.5 mg, ±0.75 mg, or ±1 mg) as the second dose (C1D2) glofitamab;
(b) a single dose (C1D1) of obinutuzumab; and
(c) one or more chemotherapeutic agents selected from ifosfamide, carboplatin and/or etoposide;
and administering to the subject in a second dosing cycle:
(a) a single dose (C2D1) of about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg) glofitamab
(b) a single dose (C2D1) of rituximab; and
(c) one or more chemotherapeutic agents selected from ifosfamide, carboplatin and/or etoposide.

In one embodiment, the single dose C1D1 of the obinutuzumab is about 1000 mg (e.g., 1000 mg ±5 mg, ±10 mg, ±20 mg, ±30 mg, ±50 mg, ±75 mg, or ±100 mg) and the single dose of rituximab is about 375 mg/m² (e.g., 375 mg/m²±5 mg/m², ±10 mg/m², ±25 mg/m², or ±37.5 mg/m²).

In one embodiment, the anti-CD20 antibody is administered in a dosing regimen comprising at least a first dosing cycle and a second dosing cycle, wherein the first dosing cycle comprises a single dose (C1D1) of obinutuzumab on Day 1; and the second dosing cycle comprises a single dose (C2D1) of rituximab on Day 1.

In one embodiment, the first dose (C1D1) of glofitamab and the second dose (C1D2) of glofitamab are administered to the subject on or about Days 8 (±1 day) and 15 (±1 day), respectively, of the first dosing cycle.

In one embodiment, the first dose (C1D1) of glofitamab and the second dose (C1D2) of glofitamab are administered to the subject on Days 8 and 15, respectively, of the first dosing cycle. In some embodiments, the C2D1 of glofitamab is administered to the subject on or about Day 8 (±1 day) of the second dosing cycle. In some embodiments, the C2D1 of glofitamab is administered to the subject on or about Day 8 of the second dosing cycle.

In one embodiment of the methods provided above, step c) comprises all three chemotherapeutic agents.

In one embodiment, the first dosing cycle comprises a single dose (C1D1) of ifosfamide, a single dose (C1D1) of carboplatin and a first (C1D1), second (C1D2) and third (C1D3) dose of etoposide; and the second cycle each comprises a single dose (C2D1) of ifosfamide, a single dose (C2D1) of carboplatin and a first (C2D1), second (C2D2) and third (C2D3) dose of etoposide.

In one aspect, the invention features a method of treating a subject having a CD20-positive cell proliferative disorder, e.g., a B cell proliferative disorder (e.g., an NHL (e.g., a relapsed and/or refractory NHL, a DLBCL (e.g., a relapsed and/or refractory DLBCL), a FL (e.g., a relapsed and/or refractory FL or a transformed FL), or an MCL (e.g., a relapsed or refractory MCL)), or a CNSL), comprising administering to the subject in a first dosing cycle:
(a) about 2.5 mg (e.g., 2.5 mg±0.01 mg, ±0.02 mg, ±0.03 mg, ±0.05 mg, ±0.1 mg, ±0.2 mg, or ±0.25 mg) as the first dose (C1D1) of glofitamab and about 10 mg (e.g., 10 mg±0.05 mg, ±0.1 mg, ±0.2 mg, ±0.3 mg, ±0.5 mg, ±0.75 mg, or ±1 mg) as the second dose (C1D2) of glofitamab;
(b) a single dose (C1D1) of obinutuzumab; and
(c) a single dose (C1D1) of ifosfamide, a single dose (C1D1) of carboplatin and a first (C1D1), second (C1D2) and third (C1D3) dose of etoposide;
and administering to the subject in a second dosing cycle
(a) a single dose (C2D1) of about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg) of glofitamab;
(b) a single dose (C2D1) of rituximab; and
(c) a single dose (C2D1) of ifosfamide, a single dose (C2D1) of carboplatin and a first (C2D1), second (C2D2) and third (C2D3) dose of etoposide.

In one embodiment, the single dose C1D1 of the obinutuzumab is about 1000 mg (e.g., 1000 mg ±5 mg, ±10 mg, ±20 mg, ±30 mg, ±50 mg, ±75 mg, or ±100 mg) and the single dose of rituximab is about 375 mg/m² (e.g., 375 mg/m²±5 mg/m², ±10 mg/m², ±25 mg/m², or ±37.5 mg/m²).

In one embodiment, the anti-CD20 antibody is administered in a dosing regimen comprising at least a first dosing cycle and a second dosing cycle, wherein the first dosing cycle comprises a single dose (C1D1) of obinutuzumab on Day 1; and the second dosing cycle comprises a single dose (C2D1) of rituximab on Day 1.

In one embodiment, the first dose (C1D1) of glofitamab and the second dose (C1D2) glofitamab are administered to the subject on or about Days 8 (±1 day) and 15 (±1 day), respectively, of the first dosing cycle.

In one embodiment, the first dose (C1D1) of glofitamab and the second dose (C1D2) of glofitamab are administered to the subject on Days 8 and 15, respectively, of the first dosing cycle. In some embodiments, the C2D1 of glofitamab is administered to the subject on or about Day 8 (±1 day) of the second dosing cycle. In some embodiments, the C2D1 of glofitamab is administered to the subject on or about Day 8 of the second dosing cycle.

In one embodiment of the methods provided above ifosfamide is administered at a dose of 5000 mg/m², 4000 mg/m² or 1666 mg/m². In one embodiment of the methods provided above ifosfamide is administered at a dose of 5000 mg/m². In one embodiment of the methods provided above, carboplatin is administered at a dose in mg to target area under the curve (AUC) of 5 mg/mL/min with maximum dose of 750 mg. In one embodiment of the methods provided above etoposide is administered at a dose of 100 mg/m² or 75 mg/m². In one embodiment of the methods provided above etoposide is administered at a dose of 100 mg/m².

In one embodiment of the methods provided above ifosfamide is administered at a dose of 5000 mg/m², 4000 mg/m² or 1666 mg/m², carboplatin is administered at a dose in mg to target area under the curve (AUC) of 5 mg/mL/min with maximum dose of 750 mg and etoposide is administered at a dose of 100 mg/m² or 75 mg/m².

In one embodiment of the methods provided above ifosfamide is administered at a dose of 5000 mg/m², carboplatin is administered at a dose in mg to target area under the curve (AUC) of 5 mg/mL/min with maximum dose of 750 mg and etoposide is administered at a dose of 100 mg/m².

In one embodiment, ifosfamide and carboplatin are administered on Day 2 of the first and second dosing cycle and etoposide is administered on each of Days 1, 2 and 3 of the first and second dosing cycle.

In one aspect, the invention features a method of treating a subject having a CD20-positive cell proliferative disorder, e.g., a B cell proliferative disorder (e.g., an NHL (e.g., a relapsed and/or refractory NHL, a DLBCL (e.g., a relapsed and/or refractory DLBCL), a FL (e.g., a relapsed and/or refractory FL or a transformed FL), or an MCL (e.g., a relapsed or refractory MCL)), or a CNSL), comprising administering to the subject in a first dosing cycle:
(a) about 2.5 mg (e.g., 2.5 mg±0.01 mg, ±0.02 mg, ±0.03 mg, ±0.05 mg, ±0.1 mg, ±0.2 mg, or ±0.25 mg) as the first dose (C1D1) of glofitamab and about 10 mg (e.g., 10 mg±0.05 mg, ±0.1 mg, ±0.2 mg, ±0.3 mg, ±0.5 mg, ±0.75 mg, or ±1 mg) as the second dose (C1D2) of glofitamab;
(b) a single dose (C1D1) of about 1000 mg (e.g., 1000 mg±5 mg, ±10 mg, ±20 mg, ±30 mg, ±50 mg, ±75 mg, or ±100 mg) obinutuzumab; and
(c) a single dose (C1D1) of about 5000 mg/m² (e.g., 5000 mg/m²±50 mg/m², ±100 mg/m², ±200 mg/m², ±300 mg/m², ±400 mg/m², or ±500 mg/m²) ifosfamide, a single dose (C1D1) of carboplatin at a dose in mg to target area under the curve (AUC) of 5 mg/mL/min with maximum dose of 750 mg and a first (C1D1), second (C1D2) and third (C1D3) dose of about 100 mg/m² etoposide;
and administering to the subject in a second dosing cycle:
(a) a single dose (C2D1) of about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg) of glofitamab;
(b) a single dose (C2D1) of about 375 mg/m² (e.g., 375 mg/m²±5 mg/m², ±10 mg/m², ±25 mg/m², or ±37.5 mg/m²) rituximab; and
(c) a single dose (C1D1) of about 5000 mg/m² (e.g., 5000 mg/m²±50 mg/m², ±100 mg/m², ±200 mg/m², ±300 mg/m², ±400 mg/m², or ±500 mg/m²) ifosfamide, a single dose (C1D1) of carboplatin at a dose in mg to target area under the curve (AUC) of 5 mg/mL/min with maximum dose of 750 mg and a first (C1D1), second (C1D2) and third (C1D3) dose of about 100 mg/m² etoposide.

In one aspect, the invention features a method of treating a subject having a CD20-positive cell proliferative disorder, e.g., a B cell proliferative disorder (e.g., an NHL (e.g., a relapsed and/or refractory NHL, a DLBCL (e.g., a relapsed and/or refractory DLBCL), a FL (e.g., a relapsed and/or refractory FL or a transformed FL), or an MCL (e.g., a relapsed or refractory MCL)), or a CNSL), comprising administering to the subject:
(a) about 2.5 mg (e.g., 2.5 mg±0.01 mg, ±0.02 mg, ±0.03 mg, ±0.05 mg, ±0.1 mg, ±0.2 mg, or ±0.25 mg) as the first dose (C1D1) of a glofitamab on Day 8, about 10 mg (e.g., 10 mg±0.05 mg, ±0.1 mg, ±0.2 mg, ±0.3 mg, ±0.5 mg, ±0.75 mg, or ±1 mg) as the second dose (C1D2) of glofitamab on Day 15 of the first dosing cycle and about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg) as the first dose (C2D1) on Day 8 of the second dosing cycle;
(b) a single dose (C1D1) of about 1000 mg (e.g., 1000 mg±5 mg, ±10 mg, ±20 mg, ±30 mg, ±50 mg, ±75 mg, or ±100 mg) obinutuzumab on Day 1 of the first dosing cycle and a single dose (C2D1) of about 375 mg/m² (e.g., 375 mg/m²±5 mg/m², ±10 mg/m², ±25 mg/m², or ±37.5 mg/m²) rituximab on Day 1 of the second dosing cycle; and
(c) a single dose (C1D1) of about 5000 mg/m² (e.g., 5000 mg/m²±50 mg/m², ±100 mg/m², ±200 mg/m², ±300 mg/m², ±400 mg/m², or ±500 mg/m²) ifosfamide on Day 2 of the first and second dosing cycle, a single dose (C1D1) of carboplatin at a dose in mg to target area under the curve (AUC) of 5 mg/mL/min with maximum dose of 750 mg on Day 2 of the first and second dosing cycle, and a first (C1D1), second (C1D2) and third (C1D3) dose of about 100 mg/m² etoposide on Days 1, 2 and 3 of the first and second dosing cycle.

In some embodiments, the first and second dosing cycles are 14-day (e.g., 14±3 days) dosing cycles. In some embodiments, the first and second dosing cycles are 21-day (e.g., 21±3 days) dosing cycles. In particular embodiments, the first and second dosing cycles are 21-day dosing cycles.

In some embodiments, the dosing regimen comprises one or more additional dosing cycles. In some embodiments, the dosing regimen comprises three dosing cycles in total. In some embodiments, the additional dosing cycles are 14-day (e.g., 14±3 days) dosing cycles. In some embodiments, the additional dosing cycles are 21-day (e.g., 21±3 days) dosing cycles. In one embodiment, the method provided herein comprises three 21-day dosing cycles.

In one embodiment, the one or more additional dosing cycles comprise:
(a) an additional single dose of glofitamab,
(b) an additional single dose of the anti-CD20 antibody, and
(c) an additional single dose of ifosfamide, an additional single dose of carboplatin and an additional first, second and third dose of etoposide.

In one embodiment, the additional single dose of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one embodiment, the anti-CD20 antibody is rituximab. In one embodiment, the additional single dose of rituximab is about 375 mg/m² (e.g., 375 mg/m²±5 mg/m², ±10 mg/m², ±25 mg/m², or ±37.5 mg/m²). In one embodiment, the additional single dose of rituximab is administered on Day 1 of the additional dosing cycle.

In one embodiment, the additional single dose of ifosfamide is about 5000 mg/m² (e.g., 5000 mg/m²±50 mg/m², ±100 mg/m², ±200 mg/m², ±300 mg/m², ±400 mg/m², or ±500 mg/m²), about 4000 mg/m² (e.g., 4000 mg/m²±40 mg/m², ±50 mg/m², ±100 mg/m², ±200 mg/m², ±300 mg/m², or ±400 mg/m²), or about 1666 mg/m² (e.g., 1666 mg/m²±25 mg/m², ±50 mg/m², ±100 mg/m², or ±166.6 mg/m²), the additional single dose of carboplatin is in mg to target area under the curve (AUC) of about 5 mg/mL/min (e.g., 5 mg/mL/min±0.05 mg/mL/min, ±0.1 mg/mL/min, ±0.25 mg/mL/min, or ±0.5 mg/mL/min) with maximum dose of about 750 mg (e.g., 750 mg±10 mg, ±25 mg, ±50 mg, or ±75 mg) and the additional first, second and third dose of etoposide is 100 mg/m² (e.g., 100 mg/m²±1 mg/m², ±2.5 mg/m², ±5 mg/m², or ±10 mg/m²) or 75 mg/m² (e.g., 0.5 mg/m²±1 mg/m², ±2.5 mg/m², ±5 mg/m², or ±7.5 mg/m²). In one embodiment, the additional single dose of ifosfamide is 5000 mg/m², 4000 mg/m² or 1666 mg/m², the additional single dose of carboplatin is in mg to target area under the curve (AUC) of 5 mg/mL/min with maximum dose of 750 mg and the additional first, second and third dose of etoposide is 100 mg/m² or 75 mg/m². In one embodiment, ifosfamide is administered at a dose of 5000 mg/m², carboplatin is administered at a dose in mg to target area under the curve (AUC) of 5 mg/mL/min with maximum dose of 750 mg and etoposide is administered at a dose of 100 mg/m².

In one embodiment, the additional single dose of ifosfamide is about 5000 mg/m² (e.g., 5000 mg/m²±50 mg/m², ±100 mg/m², ±200 mg/m², ±300 mg/m², ±400 mg/m², or ±500 mg/m²), about 4000 mg/m² (e.g., 4000 mg/m²±40 mg/m², ±50 mg/m², ±100 mg/m², ±200 mg/m², ±300 mg/m², or ±400 mg/m²), or about 1666 mg/m² (e.g., 1666 mg/m²±25 mg/m², ±50 mg/m², ±100 mg/m², or ±166.6 mg/m²), the additional single dose of carboplatin is about 5×(25+CreatinineClearance (CrCl)) mg with maximum dose of about 750 mg (e.g., 750 mg±10 mg, ±25 mg, ±50 mg, or ±75 mg), and the additional single dose of etoposide is about 100 mg/m² (e.g., 100 mg/m²±1 mg/m², ±2.5 mg/m², ±5 mg/m², or ±10 mg/m²) or 75 mg/m² (e.g., 0.5 mg/m²±1 mg/m², ±2.5 mg/m², ±5 mg/m², or ±7.5 mg/m²). In one embodiment, (a) the subject is male, and CrCl is calculated using the formula CrCl=([140−age]×[weight in kg])/(72×[serum creatinine in mg/dL]); or (b) the subject is female, and CrCl is calculated using the formula CrCl=0.85×([140−age]×[weight in kg])/(72×[serum creatinine in mg/dL]). In one embodiment, (a) the subject has CrCl<about 60 mL/min, and the additional single dose of ifosfamide is reduced to 4000 mg/m²; and/or (b) the subject has CrCl<about 50 mL/min, and the additional single dose of etoposide is reduced to about 75 mg/m². In one embodiment, the subject is administered or is to be administered the additional single dose of ifosfamide in an outpatient setting, and the additional single dose of ifosfamide is about 1666 mg/m² (e.g., 1666 mg/m²±25 mg/m², ±50 mg/m², ±100 mg/m², or ±166.6 mg/m²).

In one embodiment, ifosfamide and carboplatin are administered on Day 2 of the additional dosing cycle and etoposide is administered on each of Days 1, 2 and 3 of the additional dosing cycle.

In one aspect, the invention features a method of treating a subject having a CD20-positive cell proliferative disorder, e.g., a B cell proliferative disorder (e.g., an NHL (e.g., a relapsed and/or refractory NHL, a DLBCL (e.g., a relapsed and/or refractory DLBCL), a FL (e.g., a relapsed and/or refractory FL or a transformed FL), or an MCL (e.g., a relapsed or refractory MCL)), or a CNSL), comprising administering to the subject in a first dosing cycle:
(a) about 2.5 mg (e.g., 2.5 mg±0.01 mg, ±0.02 mg, ±0.03 mg, ±0.05 mg, ±0.1 mg, ±0.2 mg, or ±0.25 mg as the first dose (C1D1) of glofitamab and about 10 mg (e.g., 10 mg±0.05 mg, ±0.1 mg, ±0.2 mg, ±0.3 mg, ±0.5 mg, ±0.75 mg, or ±1 mg) as the second dose (C1D2) of glofitamab;
(b) a single dose (C1D1) of obinutuzumab; and
(c) a single dose (C1D1) of ifosfamide, a single dose (C1D1) of carboplatin and a first (C1D1), second (C1D2) and third (C1D3) dose of etoposide;
and administering to the subject in a second and third dosing cycle:
(a) a single dose (C2D1 in Cycle 2 and C3D1 in Cycle 3) of about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg) of glofitamab;
(b) a single dose (C2D1 in Cycle 2 and C3D1 in Cycle 3) of rituximab; and
(c) a single dose (C2D1 in Cycle 2 and C3D1 in Cycle 3) of ifosfamide, a single dose (C2D1 in Cycle 2 and C3D1 in Cycle 3) of carboplatin and a first (C2D1 in Cycle 2 and C3D1 in Cycle 3), second (C2D2 in Cycle 2 and C3D2 in Cycle 3) and third (C2D3 in Cycle 2 and C3D3 in Cycle 3) dose of etoposide.

In one aspect, the invention features a method of treating a subject having a CD20-positive cell proliferative disorder, e.g., a B cell proliferative disorder (e.g., an NHL (e.g., a relapsed and/or refractory NHL, a DLBCL (e.g., a relapsed and/or refractory DLBCL), a FL (e.g., a relapsed and/or refractory FL or a transformed FL), or an MCL (e.g., a relapsed or refractory MCL)), or a CNSL), comprising administering to the subject in a first dosing cycle:
(a) about 2.5 mg (e.g., 2.5 mg±0.01 mg, ±0.02 mg, ±0.03 mg, ±0.05 mg, ±0.1 mg, ±0.2 mg, or ±0.25 mg as the first dose (C1D1) of glofitamab and about 10 mg (e.g., 10 mg±0.05 mg, ±0.1 mg, ±0.2 mg, ±0.3 mg, ±0.5 mg, ±0.75 mg, or ±1 mg) as the second dose (C1D2) of the glofitamab that binds to CD20 and CD3;
(b) a single dose (C1D1) of about 1000 mg (e.g., 1000 mg±5 mg, ±10 mg, ±20 mg, ±30 mg, ±50 mg, ±75 mg, or ±100 mg) obinutuzumab; and
(c) a single dose (C1D1) of about 5000 mg/m² (e.g., 5000 mg/m²±50 mg/m², ±100 mg/m², ±200 mg/m², ±300 mg/m², ±400 mg/m², or ±500 mg/m²) ifosfamide, a single dose (C1D1) of carboplatin at a dose in mg to target area under the curve (AUC) of 5 mg/mL/min with maximum dose of 750 mg and a first (C1D1), second (C1D2) and third (C1D3) dose of about 100 mg/m² etoposide;
and administering to the subject in a second and third dosing cycle:
(a) a single dose (C2D1 in Cycle 2 and C3D1 in Cycle 3) of about 30 mg (e.g., 30 mg±0.1 mg, ±0.2 mg, ±0.3 mg, ±0.5 mg, ±0.75 mg, ±1 mg, ±1.5 0.2 mg, ±2 mg, or ±3 mg) of glofitamab;
(b) a single dose (C2D1 in Cycle 2 and C3D1 in Cycle 3) of about 375 mg/m² (e.g., 375 mg/m²±5 mg/m², ±10 mg/m², ±25 mg/m², or ±37.5 mg/m²) rituximab; and
(c) a single dose (C2D1 in Cycle 2 and C3D1 in Cycle 3) of about 5000 mg/m² (e.g., 5000 mg/m²±50 mg/m², ±100 mg/m², ±200 mg/m², ±300 mg/m², ±400 mg/m², or ±500 mg/m²) ifosfamide, a single dose (C2D1 in Cycle 2 and C3D1 in Cycle 3) of carboplatin at a dose in mg to target area under the curve (AUC) of 5 mg/mL/min with maximum dose of 750 mg and a first (C2D1 in Cycle 2 and C3D1 in Cycle 3), second (C2D2 in Cycle 2 and C3D2 in Cycle 3) and third (C2D3 in Cycle 2 and C3D3 in Cycle 3) dose of about 100 mg/m² etoposide.

In one embodiment, carboplatin is administered at a dose of about 5×(25+CreatinineClearance (CrCl)) mg with maximum dose of about 750 mg (e.g., 750 mg±10 mg, ±25 mg, ±50 mg, or ±75 mg). In one embodiment, (a) the subject is male, and CrCl is calculated using the formula CrCl=([140−age]×[weight in kg])/(72×[serum creatinine in mg/dL]); or (b) the subject is female, and CrCl is calculated using the formula CrCl=0.85×([140−age]×[weight in kg])/(72×[serum creatinine in mg/dL]).

In one aspect, the invention features a method of treating a subject having a CD20-positive cell proliferative disorder, e.g., a B cell proliferative disorder (e.g., an NHL (e.g., a relapsed and/or refractory

NHL, a DLBCL (e.g., a relapsed and/or refractory DLBCL), a FL (e.g., a relapsed and/or refractory FL or a transformed FL), or an MCL (e.g., a relapsed or refractory MCL)), or a CNSL), comprising administering to the subject:
(a) about 2.5 mg (e.g., 2.5 mg±0.01 mg, ±0.02 mg, ±0.03 mg, ±0.05 mg, ±0.1 mg, ±0.2 mg, or ±0.25 mg as the first dose (C1D1) of glofitamab on Day 8, about 10 mg (e.g., 10 mg±0.05 mg, ±0.1 mg, ±0.2 mg, ±0.3 mg, ±0.5 mg, ±0.75 mg, or ±1 mg) as the second dose (C1D2) of glofitamab on Day 15 of the first dosing cycle and about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg) as the first dose (C2D1) on Day 8 of the second and third dosing cycle;
(b) a single dose (C1D1) of about 1000 mg (e.g., 1000 mg±5 mg, ±10 mg, ±20 mg, ±30 mg, ±50 mg, ±75 mg, or ±100 mg) obinutuzumab on Day 1 of the first dosing cycle and a single dose (C2D1) of about 375 mg/m² (e.g., 375 mg/m²±5 mg/m², ±10 mg/m², ±25 mg/m², or ±37.5 mg/m²) rituximab on Day 1 of the second and third dosing cycle; and
(c) a single dose (C1D1) of about 5000 mg/m² (e.g., 5000 mg/m²±50 mg/m², ±100 mg/m², ±200 mg/m², ±300 mg/m², ±400 mg/m², or ±500 mg/m²) ifosfamide on Day 2 of the first and second dosing cycle, a single dose (C1D1) of carboplatin at a dose in mg to target area under the curve (AUC) of 5 mg/mL/min with maximum dose of 750 mg on Day 2 of all three dosing cycles, and a first (C1D1), second (C1D2) and third (C1D3) dose of about 100 mg/m² etoposide on Days 1, 2 and 3 of all three dosing cycles.

In one aspect, the invention features a method of treating a subject aged between 6 months and 17 years having a CD20-positive cell proliferative disorder comprising administering to the subject an effective amount of:
(a) a bispecific antibody that binds to CD20 and CD3;
(b) an anti-CD20 antibody; and
(c) one or more chemotherapeutic agents selected from ifosfamide, carboplatin, and/or etoposide in a dosing regimen comprising at least a first dosing cycle and a second dosing cycle.

In one embodiment, the first dosing cycle comprises a first dose (C1D1) of the bispecific antibody and a second dose (C1D2) of the bispecific antibody, wherein the C1D1 of the bispecific antibody is about 0.03 mg/kg (e.g., 0.03 mg/kg±0.0005 mg/kg, ±0.001 mg/kg, ±0.002 mg/kg, or ±0.003 mg/kg), about 0.04 mg/kg (e.g., 0.04 mg/kg±0.0005 mg/kg, ±0.001 mg/kg, ±0.002 mg/kg, ±0.003 mg/kg, or ±0.004 mg/kg), or about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of the bispecific antibody is about 0.15 mg/kg (e.g., 0.15 mg/kg±0.001 mg/kg, ±0.0025 mg/kg, ±0.005 mg/kg, ±0.01 mg/kg, or ±0.015 mg/kg) or about 10 mg (e.g., 10 mg±0.05 mg, ±0.1 mg, ±0.2 mg, ±0.3 mg, ±0.5 mg, ±0.75 mg, or ±1 mg); and
the second dosing cycle comprises a single dose (C2D1) of the bispecific antibody, wherein the C2D1 of the bispecific antibody is about 0.4 mg/kg (e.g., 0.4 mg/kg±0.005 mg/kg, ±0.01 mg/kg, ±0.02 mg/kg, ±0.03 mg/kg, or ±0.04 mg/kg), about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg), or about 30 mg (e.g., 30 mg±0.1 mg, ±0.2 mg, ±0.3 mg, ±0.5 mg, ±0.75 mg, ±1 mg, ±1.5 mg, ±2 mg, or 3 mg).

In one embodiment, (a) the subject's body weight is greater than or equal to about 7.5 kg and less than about 13 kg, and wherein the C1D1 of the bispecific antibody is about 0.04 mg/kg (e.g., 0.04 mg/kg±0.0005 mg/kg, ±0.001 mg/kg, ±0.002 mg/kg, ±0.003 mg/kg, or ±0.004 mg/kg), the C1D2 of the bispecific antibody is about 0.15 mg/kg (e.g., 0.15 mg/kg±0.001 mg/kg, ±0.0025 mg/kg, ±0.005 mg/kg, ±0.01 mg/kg, or ±0.015 mg/kg), and the C2D1 of the bispecific antibody is about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg);
(b) the subject's body weight is greater than or equal to about 13 kg and less than about 45 kg, and wherein the C1D1 of the bispecific antibody is about 0.03 mg/kg (e.g., 0.03 mg/kg±0.0005 mg/kg, ±0.001 mg/kg, ±0.002 mg/kg, or ±0.003 mg/kg), the C1D2 of the bispecific antibody is about 0.15 mg/kg (e.g., 0.15 mg/kg±0.001 mg/kg, ±0.0025 mg/kg, ±0.005 mg/kg, ±0.01 mg/kg, or ±0.015 mg/kg), and the C2D1 of the bispecific antibody is about 0.4 mg/kg (e.g., 0.4 mg/kg±0.005 mg/kg, ±0.01 mg/kg, ±0.02 mg/kg, ±0.03 mg/kg, or ±0.04 mg/kg); or
(c) the subject's body weight is greater than or equal to about 45 kg, and wherein the C1D1 of the bispecific antibody is about 2.5 mg (e.g., 2.5 mg±0.01 mg, ±0.02 mg, ±0.03 mg, ±0.05 mg, ±0.1 mg, ±0.02 mg, or ±0.25 mg), the C1D2 of the bispecific antibody is about 10 mg (e.g., 10 mg±0.05 mg, ±0.1 mg, ±0.2 mg, ±0.3 mg, ±0.5 mg, ±0.75 mg, or ±1 mg), and the C2D1 of the bispecific antibody is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one embodiment, the C1D1 of the bispecific antibody and the C1D2 of the bispecific antibody are administered to the subject on Days 8 and 15, respectively, of the first dosing cycle. In one embodiment, the C2D1 of the bispecific antibody is administered to the subject on Day 1 of the second dosing cycle.

In one embodiment, the anti-CD20 antibody is obinutuzumab and/or rituximab.

In one embodiment, the first dosing cycle comprises a first dose (C1D1) of obinutuzumab and a second dose (C1D2) of obinutuzumab. In one embodiment, (a) the subject's body weight is greater than or equal to about 7.5 kg and less than about 13 kg, and wherein the sum of the C1D1 and the C1D2 of obinutuzumab is about 38 mg/kg (e.g., 38 mg/kg±0.25 mg/kg, ±0.5 mg/kg, ±1 mg/kg, ±2 mg/kg, ±3 mg/kg, or ±3.8 mg/kg); (b) the subject's body weight is greater than or equal to about 13 kg and less than about 20 kg, and wherein the sum of the C1D1 and the C1D2 of obinutuzumab is about 28 mg/kg (e.g., 28 mg/kg±0.25 mg/kg, ±0.5 mg/kg, ±1 mg/kg, ±2 mg/kg, or ±2.8 mg/kg); (c) the subject's body weight is greater than or equal to about 20 kg and less than about 32 kg, and wherein the sum of the C1D1 and the C1D2 of obinutuzumab is about 23 mg/kg (e.g., 23 mg/kg±0.25 mg/kg, ±0.5 mg/kg, ±1 mg/kg, ±2 mg/kg, or ±2.3 mg/kg); (d) the subject's body weight is greater than or equal to about 32 kg and less than about 45 kg, and wherein the sum of the C1D1 and the C1D2 of obinutuzumab is about 20 mg/kg (e.g., 23 mg/kg±0.25 mg/kg, ±0.5 mg/kg, ±1 mg/kg, or ±2 mg/kg); or (e) the subject's body weight is greater than or equal to about 45 kg, and wherein the sum of the C1D1 and the C1D2 of obinutuzumab is about 1000 mg (e.g., 1000 mg±5 mg, ±10 mg, ±20 mg, ±30 mg, ±50 mg, ±75 mg, or ±100 mg).

In one embodiment, the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab. In one embodiment, (a) the subject's body weight is greater than or equal to about 7.5 kg and less than about 13 kg, and wherein the C1D1 of obinutuzumab is about 3.8 mg/kg (e.g., 3.8 mg/kg±0.05 mg/kg, 0.1 mg/kg, ±0.2 mg/kg, ±0.3 mg/kg, or ±0.38 mg/kg) and the C1D2 of obinutuzumab is about 34.2 mg/kg (e.g., 34.2 mg/kg±0.5 mg/kg, 1 mg/kg, ±2 mg/kg, ±3 mg/kg, or ±3.42 mg/kg); (b) the subject's body weight is greater than or equal to about 13 kg and less than about 20 kg, and wherein the C1D1 of obinutuzumab is about 2.8 mg/kg (e.g., 2.8 mg/kg±0.05 mg/kg, 0.1 mg/kg, ±0.2 mg/kg, or ±0.28 mg/kg) and the C1D2 of obinutuzumab is about 35.2 mg/kg (e.g., 35.2 mg/kg±0.5 mg/kg, 1 mg/kg, ±2 mg/kg, ±3 mg/kg, or ±3.52 mg/kg); (c) the subject's body weight is greater than or equal to about 20 kg and less than about 32 kg, and wherein the C1D1 of obinutuzumab is about 2.3 mg/kg (e.g., 2.3 mg/kg±0.05 mg/kg, 0.1 mg/kg, ±0.2 mg/kg, or ±0.23 mg/kg) and the C1D2 of obinutuzumab is about 35.7 mg/kg (e.g., 35.7 mg/kg±0.5 mg/kg, 1 mg/kg, ±2 mg/kg, ±3 mg/kg, or ±3.57 mg/kg); (d) the subject's body weight is greater than or equal to about 32 kg and less than about 45 kg, and wherein the C1D1 of obinutuzumab is about 2.0 mg/kg (e.g., 2.0 mg/kg±0.05 mg/kg, 0.1 mg/kg, or ±0.2 mg/kg) and the C1D2 of obinutuzumab is about 36.0 mg/kg (e.g., 36.0 mg/kg±0.5 mg/kg, 1 mg/kg, ±2 mg/kg, ±3 mg/kg, or ±3.6 mg/kg); or (e) the subject's body weight is greater than or equal to about 45 kg, and wherein the C1D1 of obinutuzumab is about 100 mg (e.g., 100 mg±0.5 mg, ±1 mg, ±1.5 mg, ±2 mg, ±4 mg, ±6 mg, ±8 mg, or ±10 mg) and the C1D2 of obinutuzumab is about 900 mg (e.g., 900 mg±5 mg, ±10 mg, ±20 mg, ±30 mg, ±40 mg, ±50 mg, ±60 mg, ±70 mg, ±80 mg, or ±90 mg).

In one embodiment, the C1D1 of obinutuzumab is administered to the subject on Day 1 of the first dosing cycle and the C1D2 of obinutuzumab is administered to the subject on Day 2 of the first dosing cycle.

In one embodiment, the second dosing cycle comprises a single dose (C2D1) of rituximab. In one embodiment, the C2D1 of rituximab is about 375 mg/m² (e.g., 375 mg/m²±5 mg/m², ±10 mg/m², ±25 mg/m², or ±37.5 mg/m²). In one embodiment, rituximab is administered to the subject on Day 5 of the second dosing cycle.

In one embodiment, the method comprises administering to the subject ifosfamide, carboplatin, and etoposide.

In one embodiment, the first dosing cycle comprises:
(a) a first dose (C1D1) of ifosfamide, a second dose (C1D2) of ifosfamide, and a third dose (C1D3) of ifosfamide;
(b) a single dose (C1D1) of carboplatin; and
(c) a first dose (C1D1) of etoposide, a second dose (C1D2) of etoposide, and a third dose (C1D3) of etoposide;
and the second cycle comprises:
(a) a first dose (C2D1) of ifosfamide, a second dose (C2D2) of ifosfamide, and a third dose (C2D3) of ifosfamide;
(b) a single dose (C2D1) of carboplatin; and
(c) a first dose (C2D1) of etoposide, a second dose (C2D2) of etoposide, and a third dose (C2D3) of etoposide.

In one embodiment, ifosfamide is administered at a dose of about 3000 mg/m² (e.g., 3000 mg/m²±40 mg/m², ±50 mg/m², ±100 mg/m², ±200 mg/m², or ±300 mg/m²) for each dose of ifosfamide, carboplatin is administered at a dose of about 635 mg/m² (e.g., 635 mg/m²±5 mg/m², ±10 mg/m², ±25 mg/m², ±50 mg/m², ±60 mg/m², or ±63.5 mg/m²), and etoposide is administered at a dose of about 100 mg/m² (e.g., 100 mg/m²±1 mg/m², ±2.5 mg/m², ±5 mg/m², or ±10 mg/m²) for each dose of etoposide. In one embodiment, (a) the C1D1, C1D2, and C1D3 of ifosfamide are administered on Days 3, 4, and 5, respectively of the first dosing cycle; (b) the C1D1 of carboplatin is administered on Day 3 of the first dosing cycle; (c) the C1D1, C1D2, and C1D3 of etoposide are administered on Days 3, 4, and 5, respectively, of the first dosing cycle; (d) the C2D1, C2D2, and C2D3 of ifosfamide are administered on Days 6, 7, and 8, respectively, of the second dosing cycle; (e) the C2D1 of carboplatin is administered on Day 6 of the second dosing cycle; and (f) the C2D1, C2D2, and C2D3 of etoposide are administered on Days 6, 7, and 8, respectively, of the second dosing cycle.

In one embodiment, the first and second dosing cycles are each 21-day dosing cycles. In one embodiment, the dosing regimen comprises one or more additional dosing cycles. In one embodiment, the one or more additional dosing cycles are each 21-day dosing cycles. In one embodiment, the dosing regimen comprises three dosing cycles in total.

In one embodiment, the one or more additional dosing cycles each comprises:
(a) an additional single dose of the bispecific antibody that binds to CD20 and CD3,
(b) an additional single dose of the anti-CD20 antibody, and
(c) an additional first dose, an additional second dose, and an additional third dose of ifosfamide; an additional single dose of carboplatin; and an additional first dose, an additional second dose, and an additional third dose of etoposide. In one embodiment, (a) the subject's body weight is greater than or equal to about 7.5 kg and less than about 13 kg, and wherein the additional single dose of the bispecific antibody is about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg); (b) the subject's body weight is greater than or equal to about 13 kg and less than about 45 kg, and wherein the additional single dose of the bispecific antibody is about 0.4 mg/kg (e.g., 0.4 mg/kg±0.005 mg/kg, ±0.01 mg/kg, ±0.02 mg/kg, ±0.03 mg/kg, or ±0.04 mg/kg); or (c) the subject's body weight is greater than or equal to about 45 kg, and wherein the additional single dose of the bispecific antibody is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one embodiment, the additional single dose of the bispecific antibody is administered to the subject on Day 1 of each of the one or more additional dosing cycles.

In one embodiment, the anti-CD20 antibody is rituximab. In one embodiment, the additional single dose of rituximab is about 375 mg/m² (e.g., 375 mg/m²±5 mg/m², ±10 mg/m², ±25 mg/m², or ±37.5 mg/m²). In one embodiment, the additional single dose of rituximab is administered on Day 5 of each of the one or more additional dosing cycles.

In one embodiment, the additional first dose, additional second dose, and additional third dose of ifosfamide are each about 3000 mg/m² (e.g., 3000 mg/m²±40 mg/m², ±50 mg/m², ±100 mg/m², ±200 mg/m², or ±300 mg/m²), the additional single dose of carboplatin is about 635 mg/m² (e.g., 635 mg/m²±5 mg/m², ±10 mg/m², ±25 mg/m², ±50 mg/m², ±60 mg/m², or ±63.5 mg/m²), and the additional first dose, the additional second dose, and the additional third dose of etoposide are each about 100 mg/m² (e.g., 100 mg/m²±1 mg/m², ±2.5 mg/m², ±5 mg/m², or ±10 mg/m²). In one embodiment, (a) the additional first dose, the additional second dose, and the additional third dose of ifosfamide are administered to the subject on Days 6, 7, and 8, respectively, of each of the one or more additional dosing cycles; (b) the additional single dose of carboplatin is administered on Day 6 of each of the one or more additional dosing cycles; and (c) the additional first dose, the additional second dose, and the additional third dose of etoposide are administered to the subject on Days 6, 7, and 8, respectively, of each of the one or more additional dosing cycles.

In one embodiment, the method further comprises administering to the subject one or more additional therapeutic agents.

In one embodiment, the one or more additional therapeutic agent is tocilizumab. In one embodiment, the weight of the subject is greater than or equal to about 30 kg and tocilizumab is administered at a dose of about 8 mg/kg (e.g., 8 mg/kg±0.05 mg/kg, ±0.1 mg/kg, ±0.25 mg/kg, ±0.5 mg/kg, or ±0.8 mg/kg) or the weight of the subject is less than 30 kg and tocilizumab is administered at a dose of about 12 mg/kg (e.g., 12 mg/kg±0.05 mg/kg, ±0.1 mg/kg, ±0.25 mg/kg, ±0.5 mg/kg, ±0.75 mg/kg, ±1 mg/kg, or ±1.2 mg/kg), and wherein the maximum dose is about 800 mg (e.g., 800 mg±10 mg, ±25 mg, ±50 mg, or ±80 mg).

In one embodiment, the one or more additional therapeutic agents is a corticosteroid. In one embodiment, the corticosteroid comprises prednisone, prednisolone, methylprednisolone, or dexamethasone.

In one embodiment, the corticosteroid is dexamethasone. In one embodiment, dexamethasone is administered intravenously at a dose of between about 0.15 mg/kg (e.g., 0.15 mg/kg±0.001 mg/kg, ±0.0025 mg/kg, ±0.005 mg/kg, ±0.01 mg/kg, or ±0.015 mg/kg) to about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the bispecific antibody, and wherein the maximum daily dose is 10 mg. In one embodiment, dexamethasone is administered intravenously at a dose of between about 0.15 mg/kg (e.g., 0.15 mg/kg±0.001 mg/kg, ±0.0025 mg/kg, ±0.005 mg/kg, ±0.01 mg/kg, or ±0.015 mg/kg) to about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of obinutuzumab, and wherein the maximum daily dose is 10 mg.

In one embodiment, the corticosteroid is methylprednisolone. In one embodiment, methylprednisolone is administered intravenously at a dose of between about 1 mg/kg to about 2 mg/kg (e.g., 1, 1.1, 1.2, 1.3, 1.4, 1.5, 1.6, 1.7, 1.9, or 2.0 mg/kg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the bispecific antibody. In one embodiment, methylprednisolone is administered intravenously at a dose of between about 1 mg/kg to about 2 mg/kg (e.g., 1, 1.1, 1.2, 1.3, 1.4, 1.5, 1.6, 1.7, 1.9, or 2.0 mg/kg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of obinutuzumab.

In one embodiment, the corticosteroid is prednisone or prednisolone. In one embodiment, prednisone or prednisolone is administered intravenously at a dose of about 100 mg (e.g., 100 mg±0.5 mg, ±1 mg, ±1.5 mg, ±2 mg, ±4 mg, ±6 mg, ±8 mg, or ±10 mg) or about 2 mg/kg at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the bispecific antibody. In one embodiment, prednisone or prednisolone is administered intravenously at a dose of about 100 mg (e.g., 100 mg±0.5 mg, ±1 mg, ±1.5 mg, ±2 mg, ±4 mg, ±6 mg, ±8 mg, or ±10 mg) or at least about 2 mg/kg at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of obinutuzumab.

In one embodiment, the one or more additional therapeutic agents is an antihistamine. In one embodiment, the antihistamine is diphenhydramine. In one embodiment, the subject is aged between two years and 17 years, and wherein diphenhydramine is administered intravenously at a dose of between about 10 mg to 20 mg (e.g., 10, 11, 12, 13, 14, 15, 16, 17, 18, 19, or 20 mg) with a maximum single dose of about 1.25 mg/kg. In one embodiment, the subject is aged less than two years, and wherein diphenhydramine is administered rectally at a dose of about 20 mg (e.g., 20 mg±0.1 mg, ±0.25 mg, ±0.5 mg, ±1 mg, ±1.5 mg, or ±2 mg). In one embodiment, diphenhydramine is administered at least about 30 minutes (i.e., at least 30 minutes±3 minutes; e.g., at least about 1, 2, 3, 4, 5, 6, 7, 8, 9, 10, 11, 12, 18, 24, 36, 48 hours, or more) prior to the administration of any dose of the bispecific antibody and/or the anti-CD20 antibody.

In one embodiment, the one or more additional therapeutic agents comprises granulocyte-colony stimulating factor (G-CSF). In one embodiment, G-CSF is administered between about one day and about two days (e.g., 24, 26, 28, 30, 32, 36, 38, 40, 42, 44, 46, or 48 hours) after administration of any dose of rituximab, ifosfamide, carboplatin, and/or etoposide. In one embodiment, G-CSF is administered intravenously or subcutaneously at a dose of about 5 μg/kg/day (e.g., 5 μg/kg/day±0.05 μg/kg/day, ±0.1 μg/kg/day, ±0.2 μg/kg/day, ±0.3 μg/kg/day, ±0.4 μg/kg/day, ±0.5 μg/kg/day), or about 10 μg/kg/day (e.g., 10 μg/kg/day±0.1 μg/kg/day, ±0.2 μg/kg/day, ±0.4 μg/kg/day, ±0.6 μg/kg/day, ±0.8 μg/kg/day, ±1 μg/kg/day). In one embodiment, G-CSF is administered at a dose of about 5 μg/kg/day (e.g., 5 μg/kg/day±0.05 μg/kg/day, ±0.1 μg/kg/day, ±0.2 μg/kg/day, ±0.3 μg/kg/day, ±0.4 μg/kg/day, ±0.5 μg/kg/day) in the first dosing cycle and about 10 μg/kg/day (e.g., 10 μg/kg/day±0.1 μg/kg/day, ±0.2 μg/kg/day, ±0.4 μg/kg/day, ±0.6 μg/kg/day, ±0.8 μg/kg/day, ±1 μg/kg/day) in the second dosing cycle and/or each additional dosing cycle.

In one embodiment, the one or more additional therapeutic agents is an antipyretic. In one embodiment, the antipyretic is acetaminophen or paracetamol. In one embodiment, acetaminophen or paracetamol is administered orally or intravenously at a dose of between about 500 to about 1000 mg (e.g., 500, 550, 600, 650, 700, 750, 800, 850, 900, 950, or 1000 mg). In one embodiment, acetaminophen or paracetamol is administered at least about 30 minutes (i.e., at least 30 minutes±3 minutes; e.g., at least about 1, 2, 3, 4, 5, 6, 7, 8, 9, 10, 11, 12, 18, 24, 36, 48 hours, or more) prior to the administration of any dose of the bispecific antibody and/or the anti-CD20 antibody.

In one embodiment, the one or more additional therapeutic agents is mesna. In one embodiment, mesna is administered intravenously daily as five doses totaling 3000 mg/m² in amount. In one embodiment, mesna is administered intravenously at a first dose of about 600 mg/m² prior to the administration of any dose of ifosfamide and at four repeated doses of about 600 mg/m² each at about three hours, about six hours, about nine hours, and about 12 hours, respectively, after the first dose of ifosfamide. In one embodiment, mesna is administered daily to the subject on Days 3, 4, and 5 of the first dosing cycle, on Days 6, 7, and 8 of the second dosing cycle, and/or on Days 6, 7, and 8 of each additional dosing cycle.

In one aspect, the invention features a method of treating a subject aged between 18 years and 30 years having a CD20-positive cell proliferative disorder comprising administering to the subject an effective amount of:
(a) a bispecific antibody that binds to CD20 and CD3;
(b) an anti-CD20 antibody; and
(c) one or more chemotherapeutic agents selected from ifosfamide, carboplatin, and/or etoposide in a dosing regimen comprising at least a first dosing cycle and a second dosing cycle.

In one embodiment, the first dosing cycle comprises a first dose (C1D1) of the bispecific antibody and a second dose (C1D2) of the bispecific antibody, wherein the C1D1 of the bispecific antibody is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of the bispecific antibody is about 10 mg (e.g., 10 mg±0.05 mg, ±0.1 mg, ±0.2 mg, ±0.3 mg, ±0.5 mg, ±0.75 mg, or ±1 mg); and
the second dosing cycle comprises a single dose (C2D1) of the bispecific antibody, wherein the C2D1 of the bispecific antibody is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one embodiment, the C1D1 of the bispecific antibody and the C1D2 of the bispecific antibody are administered to the subject on Days 8 and 15, respectively, of the first dosing cycle.

In one embodiment, the C2D1 of the bispecific antibody is administered to the subject on Day 1 of the second dosing cycle.

In one embodiment, the anti-CD20 antibody is obinutuzumab and/or rituximab.

In one embodiment, the first dosing cycle comprises a first dose (C1D1) of obinutuzumab and a second dose (C1D2) of obinutuzumab. In one embodiment, the sum of the C1D1 and the C1D2 of obinutuzumab is about 1000 mg (e.g., 1000 mg±5 mg, ±10 mg, ±20 mg, ±30 mg, ±50 mg, ±75 mg, or ±100 mg). In one embodiment, the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab. In one embodiment, the C1D1 of obinutuzumab is about 100 mg (e.g., 100 mg±0.5 mg, ±1 mg, ±1.5 mg, ±2 mg, ±4 mg, ±6 mg, ±8 mg, or ±10 mg) and the C1D2 of obinutuzumab is about 900 mg (e.g., 900 mg±5 mg, ±10 mg, ±20 mg, ±30 mg, ±40 mg, ±50 mg, ±60 mg, ±70 mg, ±80 mg, or ±90 mg). In one embodiment, the C1D1 of obinutuzumab is administered to the subject on Day 1 of the first dosing cycle and the C1D2 of obinutuzumab is administered to the subject on Day 2 of the first dosing cycle.

In one embodiment, the second dosing cycle comprises a single dose (C2D1) of rituximab. In one embodiment, the C2D1 of rituximab is about 375 mg/m² (e.g., 375 mg/m²±5 mg/m², ±10 mg/m², ±25 mg/m², or ±37.5 mg/m²). In one embodiment, the C2D1 of rituximab is administered to the subject on Day 5 of the second dosing cycle.

In one embodiment, the method comprises administering to the subject ifosfamide, carboplatin, and etoposide. In one embodiment, the first dosing cycle comprises:
(a) a single dose (C1D1) of ifosfamide;
(b) a single dose (C1D1) of carboplatin; and
(c) a first dose (C1D1) of etoposide, a second dose (C1D2) of etoposide, and a third dose (C1D3) of etoposide;
and the second cycle comprises:
(a) a single dose (C2D1) of ifosfamide;
(b) a single dose (C2D1) of carboplatin; and
(c) a first dose (C2D1) of etoposide, a second dose (C2D2) of etoposide, and a third dose (C2D3) of etoposide.

In one embodiment, ifosfamide is administered at a dose of about 5000 mg/m² (e.g., 5000 mg/m²±50 mg/m², ±100 mg/m², ±200 mg/m², ±300 mg/m², ±400 mg/m², or ±500 mg/m²), carboplatin is administered at a dose of about 5×(25+CreatinineClearance (CrCl)) mg with maximum dose of about 750 mg (e.g., 750 mg±10 mg, ±25 mg, ±50 mg, or ±75 mg), and etoposide is administered at a dose of about 100 mg/m² (e.g., 100 mg/m²±1 mg/m², ±2.5 mg/m², ±5 mg/m², or ±10 mg/m²) for each dose of etoposide.

In one embodiment, (a) the subject is male, and CrCl is calculated using the formula CrCl=([140−age]×[weight in kg])/(72×[serum creatinine in mg/dL]); or (b) the subject is female, and CrCl is calculated using the formula CrCl=0.85×([140−age]×[weight in kg])/(72×[serum creatinine in mg/dL]).

In one embodiment, (a) the subject has CrCl<about 60 mL/min, and each single dose of ifosfamide is reduced to 4000 mg/m²; and/or (b) the subject has CrCl<about 50 mL/min, and each dose of etoposide is reduced to about 75 mg/m².

In one embodiment, (a) the C1D1 ifosfamide is administered on Day 3 of the first dosing cycle;
(b) the C1D1 of carboplatin is administered on Day 3 of the first dosing cycle;
(c) the C1D1, C1D2, and C1D3 of etoposide are administered on Days 3, 4, and 5, respectively, of the first dosing cycle;
(d) the C2D1 of ifosfamide is administered on Day 6 of the second dosing cycle;
(e) the C2D1 of carboplatin is administered on Day 6 of the second dosing cycle; and
(f) the C2D1, C2D2, and C2D3 of etoposide are administered on Days 6, 7, and 8, respectively, of the second dosing cycle.

In one embodiment, the first and second dosing cycles are each 21-day dosing cycles. In one embodiment, the dosing regimen comprises one or more additional dosing cycles. In one embodiment, the one or more additional dosing cycles are each 21-day dosing cycles. In one embodiment, the dosing regimen comprises three dosing cycles in total.

In one embodiment, the one or more additional dosing cycles each comprise:
(a) an additional single dose of the bispecific antibody that binds to CD20 and CD3,
(b) an additional single dose of the anti-CD20 antibody, and
(c) an additional single dose of ifosfamide; an additional single dose of carboplatin; and an additional first dose, an additional second dose, and an additional third dose of etoposide.

In one embodiment, the additional single dose of the bispecific antibody is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg). In one embodiment, the additional single dose of the bispecific antibody is administered to the subject on Day 1 of each of the one or more additional dosing cycles.

In one embodiment, the anti-CD20 antibody is rituximab. In one embodiment, the additional single dose of rituximab is about 375 mg/m² (e.g., 375 mg/m²±5 mg/m², ±10 mg/m², ±25 mg/m², or ±37.5 mg/m²). In one embodiment, the additional single dose of rituximab is administered on Day 5 of each of the one or more additional dosing cycles.

In one embodiment, the additional single dose of ifosfamide is about 5000 mg/m² (e.g., 5000 mg/m²±50 mg/m², ±100 mg/m², ±200 mg/m², ±300 mg/m², ±400 mg/m², or ±500 mg/m²), the additional single dose of carboplatin is about 5×(25+CreatinineClearance (CrCl)) mg with maximum dose of about 750 mg (e.g., 750 mg±10 mg, ±25 mg, ±50 mg, or ±75 mg), and the additional first dose, the additional second dose, and the additional third dose of etoposide are each about 100 mg/m² (e.g., 100 mg/m²±1 mg/m², ±2.5 mg/m², ±5 mg/m², or ±10 mg/m²).

In one embodiment, (a) the subject is male, and wherein CrCl is calculated using the formula CrCl=([140−age]×[weight in kg])/(72×[serum creatinine in mg/dL]); or (b) the subject is female, and wherein CrCl is calculated using the formula CrCl=0.85×([140−age]×[weight in kg])/(72×[serum creatinine in mg/dL]).

In one embodiment, (a) the subject has CrCl<about 60 mL/min, and the additional single dose of ifosfamide is reduced to 4000 mg/m²; and/or (b) the subject has CrCl<about 50 mL/min, and each additional dose of etoposide is reduced to about 75 mg/m².

In one embodiment, (a) the additional single dose of ifosfamide is administered on Day 6 of each of the one or more additional dosing cycles;
(b) the additional single dose of carboplatin is administered on Day 6 of each of the one or more additional dosing cycles; and
(c) the additional first dose, the additional second dose, and the additional third dose of etoposide are administered to the subject on Days 6, 7, and 8, respectively, of each of the one or more additional dosing cycles.

In one embodiment, the method further comprises administering to the subject one or more additional therapeutic agents.

In one embodiment, the one or more additional therapeutic agent is tocilizumab. In one embodiment, the weight of the subject is greater than or equal to about 30 kg and tocilizumab is administered at a dose of about 8 mg/kg (e.g., 8 mg/kg±0.05 mg/kg, ±0.1 mg/kg, ±0.25 mg/kg, ±0.5 mg/kg, or ±0.8 mg/kg) or the weight of the subject is less than 30 kg and tocilizumab is administered at a dose of about 12 mg/kg (e.g., 12 mg/kg±0.05 mg/kg, ±0.1 mg/kg, ±0.25 mg/kg, ±0.5 mg/kg, ±0.75 mg/kg, ±1 mg/kg, or ±1.2 mg/kg), and wherein the maximum dose is about 800 mg (e.g., 800 mg±10 mg, ±25 mg, ±50 mg, or ±80 mg).

In one embodiment, the one or more additional therapeutic agents is a corticosteroid.

In one embodiment, the corticosteroid comprises prednisone, prednisolone, methylprednisolone, or dexamethasone.

In one embodiment, the corticosteroid is dexamethasone. In one embodiment, dexamethasone is administered intravenously at a dose of between about 0.15 mg/kg (e.g., 0.15 mg/kg±0.001 mg/kg, ±0.0025 mg/kg, ±0.005 mg/kg, ±0.01 mg/kg, or ±0.015 mg/kg) to about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the bispecific antibody, and wherein the maximum daily dose is 10 mg. In one embodiment, dexamethasone is administered intravenously at a dose of between about 0.15 mg/kg (e.g., 0.15 mg/kg±0.001 mg/kg, ±0.0025 mg/kg, ±0.005 mg/kg, ±0.01 mg/kg, or ±0.015 mg/kg) to about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of obinutuzumab, and wherein the maximum daily dose is 10 mg.

In one embodiment, the corticosteroid is methylprednisolone. In one embodiment, methylprednisolone is administered intravenously at a dose of between about 1 mg/kg to about 2 mg/kg (e.g., 1, 1.1, 1.2, 1.3, 1.4, 1.5, 1.6, 1.7, 1.9, or 2.0 mg/kg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the bispecific antibody. In one embodiment, methylprednisolone is administered intravenously at a dose of between about 1 mg/kg to about 2 mg/kg (e.g., 1, 1.1, 1.2, 1.3, 1.4, 1.5, 1.6, 1.7, 1.9, or 2.0 mg/kg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of obinutuzumab.

In one embodiment, the corticosteroid is prednisone or prednisolone. In one embodiment, prednisone or prednisolone is administered intravenously at a dose of about 100 mg (e.g., 100 mg±0.5 mg, ±1 mg, ±1.5 mg, ±2 mg, ±4 mg, ±6 mg, ±8 mg, or ±10 mg) or about 2 mg/kg at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the bispecific antibody. In one embodiment, prednisone or prednisolone is administered intravenously at a dose of about 100 mg (e.g., 100 mg±0.5 mg, ±1 mg, ±1.5 mg, ±2 mg, ±4 mg, ±6 mg, ±8 mg, or ±10 mg) or about 2 mg/kg at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of obinutuzumab.

In one embodiment, the one or more additional therapeutic agents is an antihistamine. In one embodiment, the antihistamine is diphenhydramine. In one embodiment, diphenhydramine is administered orally or intravenously at a dose of about 50 mg (e.g., 50 mg±0.5 mg, ±1 mg, ±1.5 mg, ±2 mg, ±3 mg, ±4 mg, or ±5 mg). In one embodiment, diphenhydramine is administered at least about 30 minutes (i.e., at least 30 minutes±3 minutes; e.g., at least about 1, 2, 3, 4, 5, 6, 7, 8, 9, 10, 11, 12, 18, 24, 36, 48 hours, or more) prior to the administration of any dose of the bispecific antibody and/or the anti-CD20 antibody.

In one embodiment, the one or more additional therapeutic agents comprises granulocyte-colony stimulating factor (G-CSF). In one embodiment, G-CSF is administered between about one day and about two days (e.g., 24, 26, 28, 30, 32, 36, 38, 40, 42, 44, 46, or 48 hours) after administration of any dose of rituximab, ifosfamide, carboplatin, and/or etoposide. In one embodiment, G-CSF is administered intravenously or subcutaneously at a dose of about 5 μg/kg/day (e.g., 5 μg/kg/day±0.05 μg/kg/day, ±0.1 μg/kg/day, ±0.2 μg/kg/day, ±0.3 μg/kg/day, ±0.4 μg/kg/day, ±0.5 μg/kg/day) or about 10 μg/kg/day (e.g., 10 μg/kg/day±0.1 μg/kg/day, ±0.2 μg/kg/day, ±0.4 μg/kg/day, ±0.6 μg/kg/day, ±0.8 μg/kg/day, ±1 μg/kg/day). In one embodiment, G-CSF is administered at a dose of about 5 μg/kg/day (e.g., 5 μg/kg/day±0.05 μg/kg/day, ±0.1 μg/kg/day, ±0.2 μg/kg/day, ±0.3 μg/kg/day, ±0.4 μg/kg/day, ±0.5 μg/kg/day) in the first dosing cycle and about 10 μg/kg/day (e.g., 10 μg/kg/day±0.1 μg/kg/day, ±0.2 μg/kg/day, ±0.4 μg/kg/day, ±0.6 μg/kg/day, ±0.8 μg/kg/day, ±1 μg/kg/day) in the second dosing cycle and/or each additional dosing cycle.

In one embodiment, the one or more additional therapeutic agents is an antipyretic. In one embodiment, the antipyretic is acetaminophen or paracetamol. In one embodiment, acetaminophen or paracetamol is administered orally or intravenously at a dose of between about 500 to about 1000 mg (e.g., 500, 550, 600, 650, 700, 750, 800, 850, 900, 950, or 1000 mg). In one embodiment, acetaminophen or paracetamol is administered at least about 30 minutes (i.e., at least 30 minutes±3 minutes; e.g., at least about 1, 2, 3, 4, 5, 6, 7, 8, 9, 10, 11, 12, 18, 24, 36, 48 hours, or more) prior to the administration of any dose of the bispecific antibody and/or the anti-CD20 antibody.

In one embodiment, the one or more additional therapeutic agents is mesna. In one embodiment, mesna is administered intravenously at a dose of about 5000 mg/m² (e.g., 5000 mg/m²±50 mg/m², ±100 mg/m², ±200 mg/m², ±300 mg/m², ±400 mg/m², or ±500 mg/m²). In one embodiment, mesna is administered via continuous infusion over about 24 hours on Day 3 of the first dosing cycle, on Day 6 of the second dosing cycle, and/or on Day 6 of each additional dosing cycle. In one embodiment, mesna is administered simultaneously with any dose of ifosfamide.

In one aspect, the invention features a method of treating a subject having a CD20-positive cell proliferative disorder comprising administering to the subject an effective amount of glofitamab, obinutuzumab, rituximab, ifosfamide, carboplatin, and etoposide in a dosing regimen comprising at least a first dosing cycle and a second dosing cycle, wherein:
(a) the first dosing cycle comprises administering a first dose (C1D1) of glofitamab on Day 8 and administering a second dose (C1D2) of glofitamab on Day 15, wherein the C1D1 of glofitamab is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 10 mg (e.g., 10 mg±0.05 mg, ±0.1 mg, ±0.2 mg, ±0.3 mg, ±0.5 mg, ±0.75 mg, or ±1 mg); and
(b) the second dosing cycle comprises administering a single dose (C2D1) of glofitamab on Day 8, wherein the C2D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one aspect, the invention features a method of treating a subject having a CD20-positive cell proliferative disorder comprising administering to the subject an effective amount of glofitamab, obinutuzumab, rituximab, ifosfamide, carboplatin, and etoposide in a dosing regimen comprising a first dosing cycle, a second dosing cycle, and a third dosing cycle, wherein:
(a) the first dosing cycle comprises administering a first dose (C1D1) of glofitamab on Day 8 and administering a second dose (C1D2) of glofitamab on Day 15, wherein the C1D1 of glofitamab is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 10 mg (e.g., 10 mg±0.05 mg, ±0.1 mg, ±0.2 mg, ±0.3 mg, ±0.5 mg, ±0.75 mg, or ±1 mg);
(b) the second dosing cycle comprises administering a single dose (C2D1) of glofitamab on Day 8, wherein the C2D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or 3 mg); and
(c) the third dosing cycle comprises administering a single dose (C3D1) of glofitamab on Day 8, wherein the C3D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one aspect, the invention features a method of treating a subject having a CD20-positive cell proliferative disorder comprising administering to the subject an effective amount of glofitamab, obinutuzumab, rituximab, ifosfamide, carboplatin, and etoposide in a dosing regimen comprising at least a first dosing cycle and a second dosing cycle, wherein:
(a) the first dosing cycle comprises:
   (i) administering a first dose (C1D1) of glofitamab on Day 8 and a second dose (C1D2) of glofitamab on Day 15, wherein the C1D1 of glofitamab is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 10 mg (e.g., 10 mg±0.05 mg, ±0.1 mg, ±0.2 mg, ±0.3 mg, ±0.5 mg, ±0.75 mg, or ±1 mg);
   (ii) administering a first dose (C1D1) of obinutuzumab on Day 1, wherein the C1D1 of obinutuzumab is about 1000 mg (e.g., 1000 mg±5 mg, ±10 mg, ±20 mg, ±30 mg, ±50 mg, ±75 mg, or ±100 mg);
   (iii) administering a single dose (C1D1) of ifosfamide on Day 2, wherein the C1D1 of ifosfamide is about 5000 mg/m² (e.g., 5000 mg/m²±50 mg/m², ±100 mg/m², ±200 mg/m², ±300 mg/m², ±400 mg/m², or ±500 mg/m²) with a maximum dose of about 800 mg (e.g., 800 mg±10 mg, ±25 mg, ±50 mg, or ±80 mg);
   (iv) administering a single dose (C1D1) of carboplatin on Day 2, wherein the C1D1 of carboplatin is about 5×(25+creatinine clearance) mg; and
   (v) administering a first dose (C1D1) of etoposide on Day 1, a second dose (C1D2) of etoposide on Day 2, and a third dose (C1D3) of etoposide on Day 3, wherein the C1D1, the C1D2, and the C1D3 of etoposide are each about 100 mg/m² (e.g., 100 mg/m²±1 mg/m², ±2.5 mg/m², ±5 mg/m², or ±10 mg/m²); and
(b) the second dosing cycle comprises:
   (i) administering a single dose (C2D1) of glofitamab on Day 8, wherein the C2D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg);
   (ii) administering a first dose (C2D1) of rituximab on Day 1, wherein the C2D1 of rituximab is about 375 mg/m² (e.g., 375 mg/m²±5 mg/m², ±10 mg/m², ±25 mg/m², or ±37.5 mg/m²);
   (iii) administering a single dose (C2D1) of ifosfamide on Day 2, wherein the C2D1 of ifosfamide is about 5000 mg/m² (e.g., 5000 mg/m²±50 mg/m², ±100 mg/m², ±200 mg/m², ±300 mg/m², ±400 mg/m², or ±500 mg/m²) with a maximum dose of about 800 mg (e.g., 800 mg±10 mg, ±25 mg, ±50 mg, or ±80 mg);
   (iv) administering a single dose (C2D1) of carboplatin on Day 2, wherein the C2D1 of carboplatin is about 5×(25+creatinine clearance) mg; and
   (v) administering a first dose (C2D1) of etoposide on Day 1, a second dose (C2D2) of etoposide on Day 2, and a third dose (C2D3) of etoposide on Day 3, wherein the C2D1, the C2D2, and the C2D3 of etoposide are each about 100 mg/m² (e.g., 100 mg/m²±1 mg/m², ±2.5 mg/m², ±5 mg/m², or ±10 mg/m²).

In one aspect, the invention features a method of treating a subject having a CD20-positive cell proliferative disorder comprising administering to the subject an effective amount of glofitamab, obinutuzumab, rituximab, ifosfamide, carboplatin, and etoposide in a dosing regimen comprising a first dosing cycle, a second dosing cycle, and a third dosing cycle, wherein:
(a) the first dosing cycle comprises:
   (i) administering a first dose (C1D1) of glofitamab on Day 8 and a second dose (C1D2) of glofitamab on Day 15, wherein the C1D1 of glofitamab is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 10 mg (e.g., 10 mg±0.05 mg, ±0.1 mg, ±0.2 mg, ±0.3 mg, ±0.5 mg, ±0.75 mg, or ±1 mg);
   (ii) administering a first dose (C1D1) of obinutuzumab on Day 1, wherein the C1D1 of obinutuzumab is about 1000 mg (e.g., 1000 mg±5 mg, ±10 mg, ±20 mg, ±30 mg, ±50 mg, ±75 mg, or ±100 mg);
   (iii) administering a single dose (C1D1) of ifosfamide on Day 2, wherein the C1D1 of ifosfamide is about 5000 mg/m² (e.g., 5000 mg/m²±50 mg/m², ±100 mg/m², ±200 mg/m², ±300 mg/m², ±400 mg/m², or ±500 mg/m²) with a maximum dose of about 800 mg (e.g., 800 mg±10 mg, ±25 mg, ±50 mg, or ±80 mg);
   (iv) administering a single dose (C1D1) of carboplatin on Day 2, wherein the C1D1 of carboplatin is about 5×(25+creatinine clearance) mg; and
   (v) administering a first dose (C1D1) of etoposide on Day 1, a second dose (C1D2) of etoposide on Day 2, and a third dose (C1D3) of etoposide on Day 3, wherein the C1D1, the C1D2, and the C1D3 of etoposide are each about 100 mg/m² (e.g., 100 mg/m²±1 mg/m², ±2.5 mg/m², ±5 mg/m², or ±10 mg/m²);
(b) the second dosing cycle comprises:
   (i) administering a single dose (C2D1) of glofitamab on Day 8, wherein the C2D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg);
   (ii) administering a first dose (C2D1) of rituximab on Day 1, wherein the C2D1 of rituximab is about 375 mg/m² (e.g., 375 mg/m²±5 mg/m², ±10 mg/m², ±25 mg/m², or ±37.5 mg/m²);
   (iii) administering a single dose (C2D1) of ifosfamide on Day 2, wherein the C2D1 of ifosfamide is about 5000 mg/m² (e.g., 5000 mg/m²±50 mg/m², ±100 mg/m², ±200 mg/m², ±300 mg/m², ±400 mg/m², or ±500 mg/m²) with a maximum dose of about 800 mg (e.g., 800 mg±10 mg, ±25 mg, ±50 mg, or ±80 mg);
   (iv) administering a single dose (C2D1) of carboplatin on Day 2, wherein the C2D1 of carboplatin is about 5×(25+creatinine clearance) mg; and
   (v) administering a first dose (C2D1) of etoposide on Day 1, a second dose (C2D2) of etoposide on Day 2, and a third dose (C2D3) of etoposide on Day 3, wherein the C2D1, the C2D2, and the C2D3 of etoposide are each about 100 mg/m² (e.g., 100 mg/m²±1 mg/m², ±2.5 mg/m², ±5 mg/m², or ±10 mg/m²); and
(c) the third dosing cycle comprises:
   (i) administering a single dose (C3D1) of glofitamab on Day 8, wherein the C3D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg);
   (ii) administering a first dose (C3D1) of rituximab on Day 1, wherein the C3D1 of rituximab is about 375 mg/m² (e.g., 375 mg/m²±5 mg/m², ±10 mg/m², ±25 mg/m², or ±37.5 mg/m²);
   (iii) administering a single dose (C3D1) of ifosfamide on Day 2, wherein the C3D1 of ifosfamide is about 5000 mg/m² (e.g., 5000 mg/m²±50 mg/m², ±100 mg/m², ±200 mg/m², ±300 mg/m², ±400 mg/m², or ±500 mg/m²) with a maximum dose of about 800 mg (e.g., 800 mg±10 mg, ±25 mg, ±50 mg, or ±80 mg);
   (iv) administering a single dose (C3D1) of carboplatin on Day 2, wherein the C3D1 of carboplatin is about 5×(25+creatinine clearance) mg; and
   (v) administering a first dose (C3D1) of etoposide on Day 1, a second dose (C3D2) of etoposide on Day 2, and a third dose (C3D3) of etoposide on Day 3, wherein the C3D1, the C3D2, and the C3D3 of etoposide are each about 100 mg/m² (e.g., 100 mg/m²±1 mg/m², ±2.5 mg/m², ±5 mg/m², or ±10 mg/m²).

In one embodiment, mesna is administered simultaneously with any dose of ifosfamide. In one embodiment, mesna is administered at a dose of about 5000 mg/m² (e.g., 5000 mg/m²±50 mg/m², ±100 mg/m², ±200 mg/m², ±300 mg/m², ±400 mg/m², or ±500 mg/m²) intravenously. In one embodiment, mesna is administered via continuous infusion over about 24 hours on Day 2 of each dosing cycle.

In one aspect, the invention features a method of treating a subject aged between 6 months and 17 years having a CD20-positive cell proliferative disorder comprising administering to the subject an effective amount of glofitamab, obinutuzumab, rituximab, ifosfamide, carboplatin, and etoposide in a dosing regimen comprising at least a first dosing cycle and a second dosing cycle, wherein:
(a) the first dosing cycle comprises administering a first dose (C1D1) of glofitamab on Day 8 and administering a second dose (C1D2) of glofitamab on Day 15, wherein the C1D1 of glofitamab is about 0.03 mg/kg (e.g., 0.03 mg/kg±0.0005 mg/kg, ±0.001 mg/kg, ±0.002 mg/kg, or ±0.003 mg/kg), about 0.04 mg/kg (e.g., 0.04 mg/kg±0.0005 mg/kg, ±0.001 mg/kg, ±0.002 mg/kg, ±0.003 mg/kg, or ±0.004 mg/kg), or about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 0.15 mg/kg (e.g., 0.15 mg/kg±0.001 mg/kg, ±0.0025 mg/kg, ±0.005 mg/kg, ±0.01 mg/kg, or ±0.015 mg/kg) or about 10 mg (e.g., 10 mg±0.05 mg, ±0.1 mg, ±0.2 mg, ±0.3 mg, ±0.5 mg, ±0.75 mg, or ±1 mg); and
(b) the second dosing cycle comprises administering a single dose (C2D1) of glofitamab on Day 8, wherein the C2D1 of glofitamab is about 0.4 mg/kg (e.g., 0.4 mg/kg±0.005 mg/kg, ±0.01 mg/kg, ±0.02 mg/kg, ±0.03 mg/kg, or ±0.04 mg/kg), about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg), or about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one aspect, the invention features a method of treating a subject aged between 6 months and 17 years having a CD20-positive cell proliferative disorder comprising administering to the subject an effective amount of glofitamab, obinutuzumab, rituximab, ifosfamide, carboplatin, and etoposide in a dosing regimen comprising a first dosing cycle, a second dosing cycle, and a third dosing cycle, wherein:
(a) the first dosing cycle comprises administering a first dose (C1D1) of glofitamab on Day 8 and administering a second dose (C1D2) of glofitamab on Day 15, wherein the C1D1 of glofitamab is about 0.03 mg/kg (e.g., 0.03 mg/kg±0.0005 mg/kg, ±0.001 mg/kg, ±0.002 mg/kg, or ±0.003 mg/kg), about 0.04 mg/kg (e.g., 0.04 mg/kg±0.0005 mg/kg, ±0.001 mg/kg, ±0.002 mg/kg, ±0.003 mg/kg, or ±0.004 mg/kg), or about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 0.15 mg/kg (e.g., 0.15 mg/kg±0.001 mg/kg, ±0.0025 mg/kg, ±0.005 mg/kg, ±0.01 mg/kg, or ±0.015 mg/kg) or about 10 mg (e.g., 10 mg±0.05 mg, ±0.1 mg, ±0.2 mg, ±0.3 mg, ±0.5 mg, ±0.75 mg, or ±1 mg);
(b) the second dosing cycle comprises administering a single dose (C2D1) of glofitamab on Day 8, wherein the C2D1 of glofitamab is about 0.4 mg/kg (e.g., 0.4 mg/kg±0.005 mg/kg, ±0.01 mg/kg, ±0.02 mg/kg, ±0.03 mg/kg, or ±0.04 mg/kg), about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg), or about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg); and
(c) the third dosing cycle comprises administering a single dose (C3D1) of glofitamab on Day 8, wherein the C3D1 of glofitamab is about 0.4 mg/kg (e.g., 0.4 mg/kg±0.005 mg/kg, ±0.01 mg/kg, ±0.02 mg/kg, ±0.03 mg/kg, or ±0.04 mg/kg), about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg), or about 30 mg (e.g., 30 mg±0.1 mg, ±0.2 mg, ±0.3 mg, ±0.5 mg, ±0.75 mg, ±1 mg, ±1.5 mg, ±2 mg, or 3 mg).

In one aspect, the invention features a method of treating a subject aged between 6 months and 17 years having a CD20-positive cell proliferative disorder comprising administering to the subject an effective amount of glofitamab, obinutuzumab, rituximab, ifosfamide, carboplatin, and etoposide in a dosing regimen comprising at least a first dosing cycle and a second dosing cycle, wherein:
(a) the first dosing cycle comprises:
   (i) administering a first dose (C1D1) of glofitamab on Day 8 and a second dose (C1D2) of glofitamab on Day 15, wherein the C1D1 of glofitamab is about 0.03 mg/kg (e.g., 0.03 mg/kg±0.0005 mg/kg, ±0.001 mg/kg, ±0.002 mg/kg, or ±0.003 mg/kg), about 0.04 mg/kg (e.g., 0.04 mg/kg±0.0005 mg/kg, ±0.001 mg/kg, ±0.002 mg/kg, ±0.003 mg/kg, or ±0.004 mg/kg), or about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 0.15 mg/kg (e.g., 0.15 mg/kg±0.001 mg/kg, ±0.0025 mg/kg, ±0.005 mg/kg, ±0.01 mg/kg, or ±0.015 mg/kg) or about 10 mg (e.g., 10 mg±0.05 mg, ±0.1 mg, ±0.2 mg, ±0.3 mg, ±0.5 mg, ±0.75 mg, or ±1 mg);
   (ii) administering a first dose (C1D1) of obinutuzumab on Day 1 and a second dose (C1D2) of obinutuzumab on Day 2, wherein the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab, and wherein the sum of the C1D1 and the C1D2 of obinutuzumab is about 38 mg/kg (e.g., 38 mg/kg±0.25 mg/kg, ±0.5 mg/kg, ±1 mg/kg, ±2 mg/kg, ±3 mg/kg, or ±3.8 mg/kg), about 28 mg/kg (e.g., 28 mg/kg±0.25 mg/kg, ±0.5 mg/kg, ±1 mg/kg, ±2 mg/kg, or ±2.8 mg/kg), about 23 mg/kg (e.g., 23 mg/kg±0.25 mg/kg, ±0.5 mg/kg, ±1 mg/kg, ±2 mg/kg, or ±2.3 mg/kg), about 20 mg/kg, or about 1000 mg (e.g., 1000 mg±5 mg, ±10 mg, ±20 mg, ±30 mg, ±50 mg, ±75 mg, or ±100 mg);
   (iii) administering a first dose (C1D1) of ifosfamide on Day 3, a second dose (C1D2) of ifosfamide on Day 4, and a third dose (C1D3) of ifosfamide on Day 5, wherein the C1D1, the C1D2, and the C1D3 of ifosfamide are each about 3000 mg/m² (e.g., 3000 mg/m²±40 mg/m², ±50 mg/m², ±100 mg/m², ±200 mg/m², or ±300 mg/m²);
   (iv) administering a single dose (C1D1) of carboplatin on Day 3, wherein the C1D1 of carboplatin is about 635 mg/m² (e.g., 635 mg/m²±5 mg/m², ±10 mg/m², ±25 mg/m², ±50 mg/m², ±60 mg/m², or ±63.5 mg/m²); and
   (v) administering a first dose (C1D1) of etoposide on Day 3, a second dose (C1D2) of etoposide on Day 4, and a third dose (C1D3) of etoposide on Day 5, wherein the C1D1, the C1D2, and the C1D3 of etoposide are each about 100 mg/m² (e.g., 100 mg/m²±1 mg/m², ±2.5 mg/m², ±5 mg/m², or ±10 mg/m²); and
(b) the second dosing cycle comprises:
   (i) administering a single dose (C2D1) of glofitamab on Day 1, wherein the C2D1 of glofitamab is about 0.4 mg/kg (e.g., 0.4 mg/kg±0.005 mg/kg, ±0.01 mg/kg, ±0.02 mg/kg, ±0.03 mg/kg, or ±0.04 mg/kg), about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg), or about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg);
   (ii) administering a first dose (C2D1) of rituximab on Day 5, wherein the C2D1 of rituximab is about 375 mg/m² (e.g., 375 mg/m²±5 mg/m², ±10 mg/m², ±25 mg/m², or ±37.5 mg/m²);
   (iii) administering a first dose (C2D1) of ifosfamide on Day 6, a second dose (C2D2) of ifosfamide on Day 7, and a third dose (C2D3) of ifosfamide on Day 8, wherein the C2D1, the C2D2, and the C2D3 of ifosfamide are each about 3000 mg/m² (e.g., 3000 mg/m²±40 mg/m², ±50 mg/m², ±100 mg/m², ±200 mg/m², or ±300 mg/m²);
   (iv) administering a single dose (C2D1) of carboplatin on Day 6, wherein the C2D1 of carboplatin is about 5×(25+creatinine clearance) mg; and
   (v) administering a first dose (C2D1) of etoposide on Day 6, a second dose (C2D2) of etoposide on Day 7, and a third dose (C2D3) of etoposide on Day 8, wherein the C2D1, the C2D2, and the C2D3 of etoposide are each about 100 mg/m² (e.g., 100 mg/m²±1 mg/m², ±2.5 mg/m², ±5 mg/m², or ±10 mg/m²).

In one aspect, the invention features a method of treating a subject aged between 6 months and 17 years having a CD20-positive cell proliferative disorder comprising administering to the subject an effective amount of glofitamab, obinutuzumab, rituximab, ifosfamide, carboplatin, and etoposide in a dosing regimen comprising a first dosing cycle, a second dosing cycle, and a third dosing cycle, wherein:
(a) the first dosing cycle comprises:
   (i) administering a first dose (C1D1) of glofitamab on Day 8 and a second dose (C1D2) of glofitamab on Day 15, wherein the C1D1 of glofitamab is about 0.03 mg/kg, about 0.04 mg/kg (e.g., 0.04 mg/kg±0.0005 mg/kg, ±0.001 mg/kg, ±0.002 mg/kg, ±0.003 mg/kg, or ±0.004 mg/kg), or about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 0.15 mg/kg (e.g., 0.15 mg/kg±0.001 mg/kg, ±0.0025 mg/kg, ±0.005 mg/kg, ±0.01 mg/kg, or ±0.015 mg/kg) or about 10 mg (e.g., 10 mg±0.05 mg, ±0.1 mg, ±0.2 mg, ±0.3 mg, ±0.5 mg, ±0.75 mg, or ±1 mg);
   (ii) administering a first dose (C1D1) of obinutuzumab on Day 1 and a second dose (C1D2) of obinutuzumab on Day 2, wherein the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab, and wherein the sum of the C1D1 and the C1D2 of obinutuzumab is about 38 mg/kg (e.g., 38 mg/kg±0.25 mg/kg, ±0.5 mg/kg, ±1 mg/kg, ±2 mg/kg, ±3 mg/kg, or ±3.8 mg/kg), about 28 mg/kg (e.g., 28 mg/kg±0.25 mg/kg, ±0.5 mg/kg, ±1 mg/kg, ±2 mg/kg, or ±2.8 mg/kg), about 23 mg/kg (e.g., 23 mg/kg±0.25 mg/kg, ±0.5 mg/kg, ±1 mg/kg, ±2 mg/kg, or ±2.3 mg/kg), about 20 mg/kg (e.g., 23 mg/kg±0.25 mg/kg, ±0.5 mg/kg, ±1 mg/kg, or ±2 mg/kg), or about 1000 mg (e.g., 1000 mg±5 mg, ±10 mg, ±20 mg, ±30 mg, ±50 mg, ±75 mg, or ±100 mg);
   (iii) administering a first dose (C1D1) of ifosfamide on Day 3, a second dose (C1D2) of ifosfamide on Day 4, and a third dose (C1D3) of ifosfamide on Day 5, wherein the C1D1, the C1D2, and the C1D3 of ifosfamide are each about 3000 mg/m² (e.g., 3000 mg/m²±40 mg/m², ±50 mg/m², ±100 mg/m², ±200 mg/m², or ±300 mg/m²);
   (iv) administering a single dose (C1D1) of carboplatin on Day 3, wherein the C1D1 of carboplatin is about 635 mg/m² (e.g., 635 mg/m²±5 mg/m², ±10 mg/m², ±25 mg/m², ±50 mg/m², ±60 mg/m², or ±63.5 mg/m²); and
   (v) administering a first dose (C1D1) of etoposide on Day 3, a second dose (C1D2) of etoposide on Day 4, and a third dose (C1D3) of etoposide on Day 5, wherein the C1D1, the C1D2, and the C1D3 of etoposide are each about 100 mg/m² (e.g., 100 mg/m²±1 mg/m², ±2.5 mg/m², ±5 mg/m², or ±10 mg/m²);
(b) the second dosing cycle comprises:
   (i) administering a single dose (C2D1) of glofitamab on Day 1, wherein the C2D1 of glofitamab is about 0.4 mg/kg (e.g., 0.4 mg/kg±0.005 mg/kg, ±0.01 mg/kg, ±0.02 mg/kg, ±0.03 mg/kg, or ±0.04 mg/kg), about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg), or about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg);
   (ii) administering a first dose (C2D1) of rituximab on Day 5, wherein the C2D1 of rituximab is about 375 mg/m² (e.g., 375 mg/m²±5 mg/m², ±10 mg/m², ±25 mg/m², or ±37.5 mg/m²);
   (iii) administering a first dose (C2D1) of ifosfamide on Day 6, a second dose (C2D2) of ifosfamide on Day 7, and a third dose (C2D3) of ifosfamide on Day 8, wherein the C2D1, the C2D2, and the C2D3 of ifosfamide are each about 3000 mg/m² (e.g., 3000 mg/m²±40 mg/m², ±50 mg/m², ±100 mg/m², ±200 mg/m², or ±300 mg/m²);
   (iv) administering a single dose (C2D1) of carboplatin on Day 6, wherein the C2D1 of carboplatin is about 5×(25+creatinine clearance) mg; and
   (v) administering a first dose (C2D1) of etoposide on Day 6, a second dose (C2D2) of etoposide on Day 7, and a third dose (C2D3) of etoposide on Day 8, wherein the C2D1, the C2D2, and the C2D3 of etoposide are each about 100 mg/m² (e.g., 100 mg/m²±1 mg/m², ±2.5 mg/m², ±5 mg/m², or ±10 mg/m²); and
(c) the third dosing cycle comprises:
   (i) administering a single dose (C3D1) of glofitamab on Day 1, wherein the C3D1 of glofitamab is about 0.4 mg/kg (e.g., 0.4 mg/kg±0.005 mg/kg, ±0.01 mg/kg, ±0.02 mg/kg, ±0.03 mg/kg, or ±0.04 mg/kg), about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg), or about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg);
   (ii) administering a first dose (C3D1) of rituximab on Day 5, wherein the C3D1 of rituximab is about 375 mg/m² (e.g., 375 mg/m²±5 mg/m², ±10 mg/m², ±25 mg/m², or ±37.5 mg/m²);
   (iii) administering a first dose (C3D1) of ifosfamide on Day 6, a second dose (C3D2) of ifosfamide on Day 7, and a third dose (C3D3) of ifosfamide on Day 8, wherein the C3D1, the C3D2, and the C3D3 of ifosfamide are each about 3000 mg/m² (e.g., 3000 mg/m²±40 mg/m², ±50 mg/m², ±100 mg/m², ±200 mg/m², or ±300 mg/m²);
   (iv) administering a single dose (C3D1) of carboplatin on Day 6, wherein the C3D1 of carboplatin is about 5×(25+creatinine clearance) mg; and
   (v) administering a first dose (C3D1) of etoposide on Day 6, a second dose (C3D2) of etoposide on Day 7, and a third dose (C3D3) of etoposide on Day 8, wherein the C3D1, the C3D2, and the C3D3 of etoposide are each about 100 mg/m² (e.g., 100 mg/m²±1 mg/m², ±2.5 mg/m², ±5 mg/m², or ±10 mg/m²).

In one embodiment, (a) the subject's body weight is greater than or equal to about 7.5 kg and less than about 13 kg, and the C1D1 of glofitamab is about 0.04 mg/kg (e.g., 0.04 mg/kg±0.0005 mg/kg, ±0.001 mg/kg, ±0.002 mg/kg, ±0.003 mg/kg, or ±0.004 mg/kg), the C1D2 of glofitamab is about 0.15 mg/kg (e.g., 0.15 mg/kg±0.001 mg/kg, ±0.0025 mg/kg, ±0.005 mg/kg, ±0.01 mg/kg, or ±0.015 mg/kg), and the C2D1 and/or C3D1 of glofitamab is about 0.5 mg/kg (e.g., 0.5 mg/kg±0.005 mg/kg, ±0.01 mg/kg, ±0.02 mg/kg, ±0.03 mg/kg, ±0.04 mg/kg, or ±0.05 mg/kg); (b) the subject's body weight is greater than or equal to about 13 kg and less than about 45 kg, and the C1D1 of glofitamab is about 0.03 mg/kg (e.g., 0.03 mg/kg±0.0005 mg/kg, ±0.001 mg/kg, ±0.002 mg/kg, or ±0.003 mg/kg), the C1D2 of glofitamab is about 0.15 mg/kg (e.g., 0.15 mg/kg±0.001 mg/kg, ±0.0025 mg/kg, ±0.005 mg/kg, ±0.01 mg/kg, or ±0.015 mg/kg), and the C2D1 and/or C3D1 of glofitamab is about 0.4 mg/kg (e.g., 0.4 mg/kg±0.005 mg/kg, ±0.01 mg/kg, ±0.02 mg/kg, ±0.03 mg/kg, or ±0.04 mg/kg); or (c) the subject's body weight is greater than or equal to about 45 kg, and wherein the C1D1 of glofitamab is about 2.5 mg (e.g., 2.5 mg ±0.01 mg, ±0.02 mg, ±0.03 mg, ±0.05 mg, ±0.1 mg, ±0.2 mg, or ±0.25 mg), the C1D2 of glofitamab is about 10 mg (e.g., 10 mg±0.05 mg, ±0.1 mg, ±0.2 mg, ±0.3 mg, ±0.5 mg, ±0.75 mg, or ±1 mg), and the C2D1 and/or C3D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one embodiment, (a) the subject's body weight is greater than or equal to about 7.5 kg and less than about 13 kg, and wherein the sum of the C1D1 and the C1D2 of obinutuzumab is about 38 mg/kg (e.g., 38 mg/kg±0.25 mg/kg, ±0.5 mg/kg, ±1 mg/kg, ±2 mg/kg, ±3 mg/kg, or ±3.8 mg/kg); (b) the subject's body weight is greater than or equal to about 13 kg and less than about 20 kg, and wherein the sum of the C1D1 and the C1D2 of obinutuzumab is about 28 mg/kg (e.g., 28 mg/kg±0.25 mg/kg, ±0.5 mg/kg, ±1 mg/kg, ±2 mg/kg, or ±2.8 mg/kg); (c) the subject's body weight is greater than or equal to about 20 kg and less than about 32 kg, and wherein the sum of the C1D1 and the C1D2 of obinutuzumab is about 23 mg/kg (e.g., 23 mg/kg±0.25 mg/kg, ±0.5 mg/kg, ±1 mg/kg, ±2 mg/kg, or ±2.3 mg/kg); (d) the subject's body weight is greater than or equal to about 32 kg and less than about 45 kg, and wherein the sum of the C1D1 and the C1D2 of obinutuzumab is about 20 mg/kg (e.g., 23 mg/kg±0.25 mg/kg, ±0.5 mg/kg, ±1 mg/kg, or ±2 mg/kg); or (e) the subject's body weight is greater than or equal to about 45 kg, and wherein the sum of the C1D1 and the C1D2 of obinutuzumab is about 1000 mg (e.g., 1000 mg±5 mg, ±10 mg, ±20 mg, ±30 mg, ±50 mg, ±75 mg, or ±100 mg).

In one embodiment, the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab. In one embodiment, (a) the subject's body weight is greater than or equal to about 7.5 kg and less than about 13 kg, and wherein the C1D1 of obinutuzumab is about 3.8 mg/kg (e.g., 3.8 mg/kg±0.05 mg/kg, 0.1 mg/kg, ±0.2 mg/kg, ±0.3 mg/kg, or ±0.38 mg/kg) and the C1D2 of obinutuzumab is about 34.2 mg/kg (e.g., 34.2 mg/kg±0.5 mg/kg, 1 mg/kg, ±2 mg/kg, ±3 mg/kg, or ±3.42 mg/kg); (b) the subject's body weight is greater than or equal to about 13 kg and less than about 20 kg, and wherein the C1D1 of obinutuzumab is about 2.8 mg/kg (e.g., 2.8 mg/kg±0.05 mg/kg, 0.1 mg/kg, ±0.2 mg/kg, or ±0.28 mg/kg) and the C1D2 of obinutuzumab is about 35.2 mg/kg (e.g., 35.2 mg/kg±0.5 mg/kg, 1 mg/kg, ±2 mg/kg, ±3 mg/kg, or ±3.52 mg/kg); (c) the subject's body weight is greater than or equal to about 20 kg and less than about 32 kg, and wherein the C1D1 of obinutuzumab is about 2.3 mg/kg (e.g., 2.3 mg/kg±0.05 mg/kg, 0.1 mg/kg, ±0.2 mg/kg, or ±0.23 mg/kg) and the C1D2 of obinutuzumab is about 35.7 mg/kg (e.g., 35.7 mg/kg±0.5 mg/kg, 1 mg/kg, ±2 mg/kg, ±3 mg/kg, or ±3.57 mg/kg); (d) the subject's body weight is greater than or equal to about 32 kg and less than about 45 kg, and wherein the C1D1 of obinutuzumab is about 2.0 mg/kg (e.g., 2.0 mg/kg±0.05 mg/kg, 0.1 mg/kg, or ±0.2 mg/kg) and the C1D2 of obinutuzumab is about 36.0 mg/kg (e.g., 36.0 mg/kg±0.5 mg/kg, 1 mg/kg, ±2 mg/kg, ±3 mg/kg, or ±3.6 mg/kg); or (e) the subject's body weight is greater than or equal to about 45 kg, and wherein the C1D1 of obinutuzumab is about 100 mg (e.g., 100 mg±0.5 mg, ±1 mg, ±1.5 mg, ±2 mg, ±4 mg, ±6 mg, ±8 mg, or ±10 mg) and the C1D2 of obinutuzumab is about 900 mg (e.g., 900 mg±5 mg, ±10 mg, ±20 mg, ±30 mg, ±40 mg, ±50 mg, ±60 mg, ±70 mg, ±80 mg, or ±90 mg).

In one embodiment, mesna is administered to the subject on Days 3, 4, and 5 of the first dosing cycle, on Days 6, 7, and 8 of the second dosing cycle, and/or on Days 6, 7, and 8 of each additional dosing cycle. In one embodiment, mesna is administered intravenously daily as five doses totaling 3000 mg/m² in amount. In one embodiment, mesna is administered intravenously at a first dose of about 600 mg/m² prior to the administration of any dose of ifosfamide and at four repeated doses of about 600 mg/m² each at about three hours, about six hours, about nine hours, and about 12 hours, respectively, after the first dose of ifosfamide.

In one aspect, the invention features a method of treating a subject aged between 18 years and 30 years having a CD20-positive cell proliferative disorder comprising administering to the subject an effective amount of glofitamab, obinutuzumab, rituximab, ifosfamide, carboplatin, and etoposide in a dosing regimen comprising at least a first dosing cycle and a second dosing cycle, wherein:
(a) the first dosing cycle comprises administering a first dose (C1D1) of glofitamab on Day 8 and administering a second dose (C1D2) of glofitamab on Day 15, wherein the C1D1 of glofitamab is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 10 mg (e.g., 10 mg±0.05 mg, ±0.1 mg, ±0.2 mg, ±0.3 mg, ±0.5 mg, ±0.75 mg, or ±1 mg); and
(b) the second dosing cycle comprises administering a single dose (C2D1) of glofitamab on Day 1, wherein the C2D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one aspect, the invention features a method of treating a subject aged between 18 years and 30 years having a CD20-positive cell proliferative disorder comprising administering to the subject an effective amount of glofitamab, obinutuzumab, rituximab, ifosfamide, carboplatin, and etoposide in a dosing regimen comprising a first dosing cycle, a second dosing cycle, and a third dosing cycle, wherein:
(a) the first dosing cycle comprises administering a first dose (C1D1) of glofitamab on Day 8 and administering a second dose (C1D2) of glofitamab on Day 15, wherein the C1D1 of glofitamab is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 10 mg (e.g., 10 mg±0.05 mg, ±0.1 mg, ±0.2 mg, ±0.3 mg, ±0.5 mg, ±0.75 mg, or ±1 mg);
(b) the second dosing cycle comprises administering a single dose (C2D1) of glofitamab on Day 1, wherein the C2D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or 3 mg); and
(c) the third dosing cycle comprises administering a single dose (C3D1) of glofitamab on Day 1, wherein the C3D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one aspect, the invention features a method of treating a subject aged between 18 years and 30 years having a CD20-positive cell proliferative disorder comprising administering to the subject an effective amount of glofitamab, obinutuzumab, rituximab, ifosfamide, carboplatin, and etoposide in a dosing regimen comprising at least a first dosing cycle and a second dosing cycle, wherein:
(a) the first dosing cycle comprises:
   (i) administering a first dose (C1D1) of glofitamab on Day 8 and a second dose (C1D2) of glofitamab on Day 15, wherein the C1D1 of glofitamab is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 10 mg (e.g., 10 mg±0.05 mg, ±0.1 mg, ±0.2 mg, ±0.3 mg, ±0.5 mg, ±0.75 mg, or ±1 mg);
   (ii) administering a first dose (C1D1) of obinutuzumab on Day 1 and a second dose (C1D2) of obinutuzumab on Day 2, wherein the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab, and wherein the sum of the C1D1 and the C1D2 of obinutuzumab is about 1000 mg (e.g., 1000 mg±5 mg, ±10 mg, ±20 mg, ±30 mg, ±50 mg, ±75 mg, or ±100 mg);
   (iii) administering a single dose (C1D1) of ifosfamide on Day 3, wherein the C1D1 of ifosfamide is about 5000 mg/m² (e.g., 5000 mg/m²±50 mg/m², ±100 mg/m², ±200 mg/m², ±300 mg/m², ±400 mg/m², or ±500 mg/m²) with a maximum dose of about 800 mg (e.g., 800 mg±10 mg, ±25 mg, ±50 mg, or ±80 mg);
   (iv) administering a single dose (C1D1) of carboplatin on Day 3, wherein the C1D1 of carboplatin is about 5×(25+creatinine clearance) mg; and
   (v) administering a first dose (C1D1) of etoposide on Day 3, a second dose (C1D2) of etoposide on Day 4, and a third dose (C1D3) of etoposide on Day 5, wherein the C1D1, the C1D2, and the C1D3 of etoposide are each about 100 mg/m² (e.g., 100 mg/m²±1 mg/m², ±2.5 mg/m², ±5 mg/m², or ±10 mg/m²); and
(b) the second dosing cycle comprises:
   (i) administering a single dose (C2D1) of glofitamab on Day 1, wherein the C2D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg);
   (ii) administering a first dose (C2D1) of rituximab on Day 5, wherein the C2D1 of rituximab is about 375 mg/m² (e.g., 375 mg/m²±5 mg/m², ±10 mg/m², ±25 mg/m², or ±37.5 mg/m²);
   (iii) administering a single dose (C2D1) of ifosfamide on Day 6, wherein the C2D1 of ifosfamide is about 5000 mg/m² (e.g., 5000 mg/m²±50 mg/m², ±100 mg/m², ±200 mg/m², ±300 mg/m², ±400 mg/m², or ±500 mg/m²) with a maximum dose of about 800 mg (e.g., 800 mg±10 mg, ±25 mg, ±50 mg, or ±80 mg);
   (iv) administering a single dose (C2D1) of carboplatin on Day 6, wherein the C2D1 of carboplatin is about 5×(25+creatinine clearance) mg; and
   (v) administering a first dose (C2D1) of etoposide on Day 6, a second dose (C2D2) of etoposide on Day 7, and a third dose (C2D3) of etoposide on Day 8, wherein the C2D1, the C2D2, and the C2D3 of etoposide are each about 100 mg/m² (e.g., 100 mg/m²±1 mg/m², ±2.5 mg/m², ±5 mg/m², or ±10 mg/m²).

In one aspect, the invention features a method of treating a subject aged between 18 years and 30 years having a CD20-positive cell proliferative disorder comprising administering to the subject an effective amount of glofitamab, obinutuzumab, rituximab, ifosfamide, carboplatin, and etoposide in a dosing regimen comprising a first dosing cycle, a second dosing cycle, and a third dosing cycle, wherein:
(a) the first dosing cycle comprises:
   (i) administering a first dose (C1D1) of glofitamab on Day 8 and a second dose (C1D2) of glofitamab on Day 15, wherein the C1D1 of glofitamab is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 10 mg (e.g., 10 mg±0.05 mg, ±0.1 mg, ±0.2 mg, ±0.3 mg, ±0.5 mg, ±0.75 mg, or ±1 mg);
   (ii) administering a first dose (C1D1) of obinutuzumab on Day 1 and a second dose (C1D2) of obinutuzumab on Day 2, wherein the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab, and wherein the sum of the C1D1 and the C1D2 of obinutuzumab is about 1000 mg (e.g., 1000 mg±5 mg, ±10 mg, ±20 mg, ±30 mg, ±50 mg, ±75 mg, or 100 mg);
   (iii) administering a single dose (C1D1) of ifosfamide on Day 3, wherein the C1D1 of ifosfamide is about 5000 mg/m² (e.g., 5000 mg/m²±50 mg/m², ±100 mg/m², ±200 mg/m², ±300 mg/m², ±400 mg/m², or ±500 mg/m²) with a maximum dose of about 800 mg (e.g., 800 mg±10 mg, ±25 mg, ±50 mg, or ±80 mg);
   (iv) administering a single dose (C1D1) of carboplatin on Day 3, wherein the C1D1 of carboplatin is about 5×(25+creatinine clearance) mg; and
   (v) administering a first dose (C1D1) of etoposide on Day 3, a second dose (C1D2) of etoposide on Day 4, and a third dose (C1D3) of etoposide on Day 5, wherein the C1D1, the C1D2, and the C1D3 of etoposide are each about 100 mg/m² (e.g., 100 mg/m²±1 mg/m², ±2.5 mg/m², ±5 mg/m², or ±10 mg/m²);
(b) the second dosing cycle comprises:
   (i) administering a single dose (C2D1) of glofitamab on Day 1, wherein the C2D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg);
   (ii) administering a first dose (C2D1) of rituximab on Day 5, wherein the C2D1 of rituximab is about 375 mg/m² (e.g., 375 mg/m²±5 mg/m², ±10 mg/m², ±25 mg/m², or ±37.5 mg/m²);
   (iii) administering a single dose (C2D1) of ifosfamide on Day 6, wherein the C2D1 of ifosfamide is about 5000 mg/m² (e.g., 5000 mg/m²±50 mg/m², ±100 mg/m², ±200 mg/m², ±300 mg/m², ±400 mg/m², or ±500 mg/m²) with a maximum dose of about 800 mg (e.g., 800 mg±10 mg, ±25 mg, ±50 mg, or ±80 mg);
   (iv) administering a single dose (C2D1) of carboplatin on Day 6, wherein the C2D1 of carboplatin is about 5×(25+creatinine clearance) mg; and
   (v) administering a first dose (C2D1) of etoposide on Day 6, a second dose (C2D2) of etoposide on Day 7, and a third dose (C2D3) of etoposide on Day 8, wherein the C2D1, the C2D2, and the C2D3 of etoposide are each about 100 mg/m² (e.g., 100 mg/m²±1 mg/m², ±2.5 mg/m², ±5 mg/m², or ±10 mg/m²); and
(c) the third dosing cycle comprises:
   (i) administering a single dose (C3D1) of glofitamab on Day 1, wherein the C3D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg);
   (ii) administering a first dose (C3D1) of rituximab on Day 5, wherein the C3D1 of rituximab is about 375 mg/m² (e.g., 375 mg/m²±5 mg/m², ±10 mg/m², ±25 mg/m², or ±37.5 mg/m²);
   (iii) administering a single dose (C3D1) of ifosfamide on Day 6, wherein the C3D1 of ifosfamide is about 5000 mg/m² (e.g., 5000 mg/m²±50 mg/m², ±100 mg/m², ±200 mg/m², ±300 mg/m², ±400 mg/m², or ±500 mg/m²) with a maximum dose of about 800 mg (e.g., 800 mg±10 mg, ±25 mg, ±50 mg, or ±80 mg);
   (iv) administering a single dose (C3D1) of carboplatin on Day 6, wherein the C3D1 of carboplatin is about 5×(25+creatinine clearance) mg; and
   (v) administering a first dose (C3D1) of etoposide on Day 6, a second dose (C3D2) of etoposide on Day 7, and a third dose (C3D3) of etoposide on Day 8, wherein the C3D1, the C3D2, and the C3D3 of etoposide are each about 100 mg/m² (e.g., 100 mg/m²±1 mg/m², ±2.5 mg/m², ±5 mg/m², or ±10 mg/m²).

In one embodiment, mesna is administered simultaneously with any dose of ifosfamide. In one embodiment, mesna is administered intravenously at a dose of about 5000 mg/m² (e.g., 5000 mg/m²±50 mg/m², ±100 mg/m², ±200 mg/m², ±300 mg/m², ±400 mg/m², or ±500 mg/m²). In one embodiment, mesna is administered via continuous infusion over about 24 hours on Day 3 of the first dosing cycle, on Day 6 of the second dosing cycle, and/or on Day 6 of each additional dosing cycle. In one embodiment, the CD20-positive cell proliferative disorder is a relapsed and/or refractory DLBCL. In one embodiment, the CD20-positive cell proliferative disorder is a relapsed and/or refractory mature B cell NHL.

In some embodiments, the dosing cycles are 14-day (e.g., 14±3 days) dosing cycles. In some embodiments, the dosing cycles are 21-day (e.g., 21±3 days) dosing cycles. In particular embodiments, the dosing cycles are 21-day dosing cycles.

In some embodiments, the methods featured by the invention further comprises administering to the subject one or more additional therapeutic agents. In some embodiments, the one or more additional therapeutic agents is tocilizumab. In some embodiments, the one or more additional therapeutic agents is a corticosteroid. In some embodiments, the corticosteroid comprises prednisone, prednisolone, methylprednisolone, or dexamethasone. In some embodiments, one or more additional therapeutic agents is an antihistamine. In some embodiments, the antihistamine is diphenhydramine. In some embodiments, the one or more additional therapeutic agents comprises allopurinol and rasburicase. In some embodiments, the one or more additional therapeutic agents is an antipyretic. In some embodiments, the one or more additional therapeutic agents comprises granulocyte colony-stimulating factor (G-CSF). In some embodiments, the one or more additional therapeutic agents is mesna.

In one embodiment the anti-CD20 antibody, e.g., glofitamab is administered intravenously.

In one embodiment, the CD20-positive cell proliferative disorder is a B cell proliferative disorder. In one embodiment, the B cell proliferative disorder is a non-Hodgkin's lymphoma (NHL) or a central nervous system lymphoma (CNSL).

In one embodiment, the NHL is a diffuse-large B cell lymphoma (DLBCL), a follicular lymphoma (FL), a mantle cell lymphoma (MCL), a marginal zone lymphoma (MZL), a high-grade B cell lymphoma, a primary mediastinal (thymic) large B cell lymphoma (PMLBCL), a diffuse B cell lymphoma, or a small lymphocytic lymphoma. In one embodiment, the NHL is a Burkitt lymphoma (BL) or a Burkitt leukemia (BAL). In one embodiment, the NHL is aggressive and/or mature. In one embodiment, the NHL is relapsed and/or refractory. In one embodiment, the B cell proliferative disorder is a relapsed and/or refractory mature B cell NHL.

In one embodiment, the subject has received one prior systemic therapy. In one embodiment, the subject has received no more than one prior systemic therapy. In one embodiment, the prior systemic therapy comprises an anti-CD20 antibody and an anthracycline. In one embodiment, the subject is human. In one embodiment, the subject is transplant or CAR-T cell therapy eligible. In one embodiment, the subject receives autologous stem cell transplantation (ASCT) after completion of the dosing regimen described above. In one embodiment, the ASCT is an autologous hematopoietic stem cell transplant. In one embodiment, the subject receives allogenic hematopoietic stem cell transplant after completion of the dosing regimen as described above. In one embodiment, the subject receives CAR-T cell therapy after completion of the dosing regimen described above.

In one embodiment, rituximab may be given on the same day as glofitamab. In one embodiment the subject has had no prior IRR with rituximab or CRS with glofitamab.

In one embodiment, etoposide phosphate may be substituted for etoposide at the same dose.

In one embodiment, obinutuzumab is administered prior to ICE in the first cycle. In one embodiment, initiation of ICE (Cycle 1) is delayed one day to facilitate completion of obinutuzumab administration.

In one embodiment the subjects achieve an objective response as well as mobilization of the target dose of 2,000,000 CD34+ hematopoietic stem cells/kg typically required as a minimum for ASCT.

### (iv) Therapeutic Agents for Use in the Methods of the Invention

### A. Anti-CD20/Anti-CD3 Bispecific Antibodies

The present invention provides a new combination treatment of an anti-CD20/anti-CD3 bispecific antibody with an anti-CD20 antibody and one or more chemotherapeutic agents selected from ifosfamide, carboplatin and/or etoposide. In one embodiment, the antibody is a monoclonal antibody. In one embodiment, the anti-CD20/anti-CD3 bispecific antibody is a polyclonal antibody. In one embodiment the anti-CD20/anti-CD3 bispecific antibody is a human antibody. In one embodiment, the anti-CD20/anti-CD3 bispecific antibody is humanized antibody. In one embodiment the anti-CD20/anti-CD3 bispecific antibody is a chimeric antibody. In one embodiment the anti-CD20/anti-CD3 bispecific antibody is full-length antibody. In one embodiment the anti-CD20/anti-CD3 bispecific antibody is an IgG-class antibody, particularly an IgG1 subclass antibody. In one embodiment, the anti-CD20/anti-CD3 bispecific antibody is a recombinant antibody.

In certain embodiments, the anti-CD20/anti-CD3 bispecific antibody comprises an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Plückthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046. In one embodiment, the antibody fragment is a Fab fragment or a scFv fragment.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as production by recombinant host cells (e.g., E. coli or phage), as described herein.

In certain embodiments, the anti-CD20/anti-CD3 bispecific antibody is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, the anti-CD20/anti-CD3 bispecific antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

In certain embodiments, the anti-CD20/anti-CD3 bispecific antibody is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

Binding domains comprised in the anti-CD20/anti-CD3 bispecific antibody may be isolated by screening combinatorial libraries for binding moieties with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N J, 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N J, 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

Techniques for making bispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (scFv) dimers (see, e.g., Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g., US 2006/0025576A1).

The anti-CD20/anti-CD3 bispecific antibody herein also includes a “Dual Acting FAb” or “DAF” comprising an antigen binding site that binds to two different antigens (see, US 2008/0069820, for example).

“Crossmab” antibodies are also included herein (see e.g., WO2009080251, WO2009080252, WO2009080253, WO2009080254).

Another technique for making bispecific antibody fragments is the “bispecific T cell engager” or BiTE® approach (see, e.g., WO2004/106381, WO2005/061547, WO2007/042261, and WO2008/119567). This approach utilizes two antibody variable domains arranged on a single polypeptide. For example, a single polypeptide chain includes two single chain Fv (scFv) fragments, each having a variable heavy chain (VH) and a variable light chain (VL) domain separated by a polypeptide linker of a length sufficient to allow intramolecular association between the two domains. This single polypeptide further includes a polypeptide spacer sequence between the two scFv fragments. Each scFv recognizes a different epitope, and these epitopes may be specific for different cell types, such that cells of two different cell types are brought into proximity or tethered when each scFv is engaged with its cognate epitope. One particular embodiment of this approach includes a scFv recognizing a cell-surface antigen expressed by an immune cell, e.g., a CD3 polypeptide on a T cell, linked to another scFv that recognizes a cell-surface antigen expressed by a target cell, such as a malignant or tumor cell.

As it is a single polypeptide, the bispecific. T cell engager may be expressed using any prokaryotic or eukaryotic cell expression system known in the art, e.g., a OHO cell line. However, specific purification techniques (see, e.g., EP1691833) may be necessary to separate monomeric bispecific T cell engagers from other multimeric species, which may have biological activities other than the intended activity of the monomer. In one exemplary purification scheme, a solution containing secreted polypeptides is first subjected to a metal affinity chromatography, and polypeptides are eluted with a gradient of imidazole concentrations. This eluate is further purified using anion exchange chromatography, and polypeptides are eluted using with a gradient of sodium chloride concentrations. Finally, this eluate is subjected to size exclusion chromatography to separate monomers from multimeric species.

In certain embodiments, the anti-CD20/anti-CD3 bispecific antibody may be further modified to contain additional nonproteinaceous moieties that are known in the art and readily available. The moieties suitable for derivatization of the anti-CD20/anti-CD3 bispecific antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

The anti-CD20/anti-CD3 bispecific antibody may also be conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, the anti-CD20/anti-CD3 bispecific antibody comprises an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another embodiment, the anti-CD20/anti-CD3 bispecific antibody is conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Saponaria officinalis inhibitor, gelonin, mitogellin, restrictocin, phenomycin, enomycin, and the tricothecenes.

In another embodiment, the anti-CD20/anti-CD3 bispecific antibody is conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example Tc^{99m} or I¹²³, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of the anti-CD20/anti-CD3 bispecific antibody and a cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of a radionucleotide to an antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

In one embodiment, the anti-CD20/anti-CD3 bispecific antibody is indicated for the treatment of cancer. In one embodiment, cancer is a B-cell proliferative disorder. In one embodiment, the cancer is a CD20-positive B-cell proliferative disorder. In one embodiment, the cancer is a non-Hodgkin's lymphoma (NHL). In one embodiment the NHL is a diffuse large B cell lymphoma (DLBCL), a high grade B cell lymphoma (HGBCL), a DLBCL arising from FL [transformed FL; trFL], a primary mediastinal large B-cell lymphoma (PMBCL), or marginal zone lymphoma (MZL). MZL can be categorized as splenic, nodal and extra-nodal MZL. In on embodiment the NHL is a mantle cell lymphoma (MCL). In one embodiment, the NHL is a Grades 1-3a Follicular Lymphoma (FL). In one embodiment, the CD20-positive B cell proliferative disorder is a relapsed or refractory B cell proliferative disorder. In one embodiment, the relapsed or refractory B cell proliferative disorder is relapsed or refractory NHL (e.g., a relapsed or refractory DLBCL, a relapsed or refractory FL, or a relapsed or refractory MCL).

In one embodiment, the anti-CD20/anti-CD3 bispecific antibody specifically binds to CD3ε.

In one embodiment, the anti-CD20/anti-CD3 bispecific antibody can compete for binding with antibody H2C (PCT publication no. WO2008/119567), antibody V9 (Rodrigues et al., Int J Cancer Suppl. 7, 45-50 (1992) and U.S. Pat. No. 6,054,297), antibody FN18 (Nooij et al., Eur J Immunol. 19, 981-984 (1986)), antibody SP34 (Pessano et al., EMBO J. 4, 337-340 (1985)), antibody OKT3 (Kung et al., Science 206, 347-349 (1979)), antibody WT31 (Spits et al., J Immunol. 135, 1922 (1985)), antibody UCHT1 (Burns et al., J Immunol. 129, 1451-1457 (1982)), antibody 7D6 (Coulie et al., Eur J Immunol. 21, 1703-1709 (1991)) or antibody Leu-4. In some embodiments, the anti-CD20/anti-CD3 bispecific antibody may also comprise an antigen binding moiety that specifically binds to CD3 as described in WO 2005/040220, WO 2005/118635, WO 2007/042261, WO 2008/119567, WO 2008/119565, WO 2012/162067, WO 2013/158856, WO 2013/188693, WO 2013/186613, WO 2014/110601, WO 2014/145806, WO 2014/191113, WO 2014/047231, WO 2015/095392, WO 2015/181098, WO 2015/001085, WO 2015/104346, WO 2015/172800, WO 2016/020444, or WO 2016/014974.

In some embodiments, the anti-CD20/anti-CD3 bispecific antibody may comprise an antibody or an antigen binding moiety from rituximab, obinutuzumab ocrelizumab, ofatumumab, ocaratuzumab, veltuzumab, and ublituximab.

In one embodiment, the anti-CD20/anti-CD3 bispecific antibody is XmAb®13676. in one embodiment, the anti-CD20/anti-CD3 bispecific antibody is REGN1979. In one embodiment, the anti-CD20/anti-CD3 bispecific antibody is FBTA05 (Lymphomun). In one embodiment, the anti-CD20/anti-CD3 bispecific antibody is glofitamab.

In some embodiments, the anti-CD20/anti-CD3 bispecific antibody may comprise a generic, biosimilar or non-comparable biologic version of an antibody, named herein.

In one embodiment, the anti-CD20/anti-CD3 bispecific antibody comprises at least one antigen binding domain that specifically binds to CD20, comprising a heavy chain variable region comprising
(i) an HVR-H1 comprising the amino acid sequence of SEQ ID NO: 1;
(ii) an HVR-H2 comprising the amino acid sequence of SEQ ID NO: 2; and
(iii) an HVR-H3 comprising the amino acid sequence of SEQ ID NO:3;
and a light chain variable region comprising
(i) an HVR-L1 comprising the amino acid sequence of SEQ ID NO: 4;
(ii) an HVR-L2 comprising the amino acid sequence of SEQ ID NO: 5; and
(iii) an HVR-L3 comprising the amino acid sequence of SEQ ID NO: 6.

In one embodiment, anti-CD20/anti-CD3 bispecific antibody comprises at least one antigen binding domain that specifically binds to CD20, comprising a heavy chain variable region sequence that is at least 80%, 85%, 90%, 95%, 96%, 97%, 98%, or 99% identical to of SEQ ID NO: 7 and a light chain variable region sequence that is at least 80%, 85%, 90%, 95%, 96%, 97%, 98%, or 99% identical to the sequence of SEQ ID NO: 8. In a further embodiment, the anti-CD20/anti-CD3 bispecific antibody comprises at least one antigen binding domain that specifically binds to CD20 comprising the heavy chain variable region sequence of SEQ ID NO: 7 and the light chain variable region sequence of SEQ ID NO: 8.

In one embodiment, the anti-CD20/anti-CD3 bispecific antibody comprises at least one antigen binding domain that specifically binds to CD3 comprising a heavy chain variable region comprising:
(i) an HVR-H1 comprising the amino acid sequence of SEQ ID NO: 9;
(ii) an HVR-H2 comprising the amino acid sequence of SEQ ID NO: 10; and
(iii) an HVR-H3 comprising the amino acid sequence of SEQ ID NO:11;
and a light chain variable region comprising
(i) an HVR-L1 comprising the amino acid sequence of SEQ ID NO: 12;
(ii) an HVR-L2 comprising the amino acid sequence of SEQ ID NO: 13; and
(iii) an HVR-L3 comprising the amino acid sequence of SEQ ID NO: 14.

In one embodiment, anti-CD20/anti-CD3 bispecific antibody comprises at least one antigen binding domain that specifically binds to CD3, comprising a heavy chain variable region sequence that is at least 80%, 85%, 90%, 95%, 96%, 97%, 98%, or 99% identical to of SEQ ID NO: 15 and a light chain variable region sequence that is at least 80%, 85%, 90%, 95%, 96%, 97%, 98%, or 99% identical to the sequence of SEQ ID NO: 16. In a further embodiment, the anti-CD20/anti-CD3 bispecific antibody comprises at least one antigen binding domain that specifically binds to CD3 comprising the heavy chain variable region sequence of SEQ ID NO: 15 and the light chain variable region sequence of SEQ ID NO: 16.

In one embodiment, the anti-CD20/anti-CD3 bispecific antibody comprises
(i) at least one antigen binding domain that specifically binds to CD20 comprising a heavy chain variable region comprising:
   a) an HVR-H1 comprising the amino acid sequence of SEQ ID NO: 1;
   b) an HVR-H2 comprising the amino acid sequence of SEQ ID NO: 2; and
   c) an HVR-H3 comprising the amino acid sequence of SEQ ID NO:3;
and a light chain variable region comprising:
   a) an HVR-L1 comprising the amino acid sequence of SEQ ID NO: 4;
   b) an HVR-L2 comprising the amino acid sequence of SEQ ID NO: 5; and
   c) an HVR-L3 comprising the amino acid sequence of SEQ ID NO: 6; and
(ii) at least one antigen binding domain that specifically binds to CD3 comprising a heavy chain variable region comprising:
   a) an HVR-H1 comprising the amino acid sequence of SEQ ID NO: 9;
   b) an HVR-H2 comprising the amino acid sequence of SEQ ID NO: 10; and
   c) an HVR-H3 comprising the amino acid sequence of SEQ ID NO:11; and
a light chain variable region comprising:
   a) an HVR-L1 comprising the amino acid sequence of SEQ ID NO: 12;
   b) an HVR-L2 comprising the amino acid sequence of SEQ ID NO: 13; and
   c) an HVR-L3 comprising the amino acid sequence of SEQ ID NO: 14.

In one embodiment, the anti-CD20/anti-CD3 bispecific antibody comprises
(i) at least one antigen binding domain that specifically binds to CD20 comprising the heavy chain variable region sequence of SEQ ID NO: 7 and the light chain variable region sequence of SEQ ID NO: 8, and
(ii) at least one antigen binding domain that specifically binds to CD3 comprising the heavy chain variable region sequence of SEQ ID NO: 15 and the light chain variable region sequence of SEQ ID NO: 16.

In one embodiment, the antigen binding domain that specifically binds to CD3 of the anti-CD20/anti-CD3 bispecific antibody is an antibody fragment, particularly a Fab molecule or a scFv molecule, more particularly a Fab molecule. In a particular embodiment, the antigen binding domain that specifically binds to CD3 of the anti-CD20/anti-CD3 bispecific antibody is a crossover Fab molecule wherein the variable domains or the constant domains of the Fab heavy and light chain are exchanged (i.e., replaced by each other).

In one embodiment, the anti-CD20/anti-CD3 bispecific antibody comprises at least one antigen binding domain that specifically binds to CD20, and one antigen binding domain that specifically binds to CD3. In one embodiment, the anti-CD20/anti-CD3 bispecific antibody comprises a first antigen binding domain that specifically binds to CD3, and a second and a third antigen binding domain that specifically bind to CD20. In one embodiment, the first antigen binding domain is a crossover Fab molecule, and the second and the third antigen binding domain are each a conventional Fab molecule. In one embodiment, the anti-CD20/anti-CD3 bispecific antibody further comprises an Fc domain. The anti-CD20/anti-CD3 bispecific antibody may comprise modifications in the Fc region and/or the antigen binding domains as described herein. In one embodiment, the anti-CD20/anti-CD3 bispecific antibody comprises an IgG1 Fc domain comprising one or more amino acid substitutions that reduce binding to an Fc receptor and/or effector function. In one embodiment the anti-CD20/anti-CD3 bispecific antibody comprises an IgG1 Fc domain comprising the amino acid substitutions L234A, L235A and P329G (EU numbering).

In one embodiment the anti-CD20/anti-CD3 bispecific antibody comprises
(i) an antigen binding domain that specifically binds to CD3 which is fused at the C-terminus of the Fab heavy chain to the N-terminus of the first subunit of the Fc domain;
(ii) a first antigen binding domain that specifically binds to CD20 which is fused at the C-terminus of the Fab heavy chain to the N-terminus of the Fab heavy chain of the antigen binding domain that specifically binds to CD3; and
(iii) a second antigen binding domain that specifically binds to CD20 which is fused at the C-terminus of the Fab heavy chain to the N-terminus of the second subunit of the Fc domain.
   In a particular embodiment, the anti-CD20/anti-CD3 bispecific antibody comprises
a) a first Fab molecule which specifically binds to CD3, particularly CD3 epsilon; and wherein the variable domains VL and VH of the Fab light chain and the Fab heavy chain are replaced by each other;
b) a second Fab and a third Fab molecule which specifically bind to CD20, wherein in the constant domain CL of the second Fab and third Fab molecule the amino acid at position 124 is substituted by lysine (K) (numbering according to Kabat) and the amino acid at position 123 is substituted by lysine (K) or arginine (R), particularly by arginine (R) (numbering according to Kabat), and wherein in the constant domain CH1 o of the second Fab and third Fab molecule the amino acid at position 147 is substituted by glutamic acid (E) (EU numbering) and the amino acid at position 213 is substituted by glutamic acid (E) (EU numbering); and
c) a Fc domain composed of a first and a second subunit capable of stable association.

In one embodiment, the anti-CD20/anti-CD3 bispecific antibody comprises two antigen binding domains that specifically bind to CD20 and one antigen binding domain that specifically binds to CD3. In one embodiment, the anti-CD20/anti-CD3 bispecific antibody is bivalent for CD20 and monovalent for CD3.

In one embodiment the first Fab molecule under a) is fused at the C-terminus of the Fab heavy chain to the N-terminus of one of the subunits of the Fc domain under c), the second Fab molecule under b) is fused at the C-terminus of the Fab heavy chain to the N-terminus of the heavy chain of the first Fab molecule under a), and the third Fab molecule under b) is fused at the C-terminus of the Fab heavy chain to the N-terminus of the other subunit of the Fc domain under c). In one embodiment, the first Fab molecule under a) comprises a heavy chain variable region that is at least 95%, 96%, 97%, 98%, or 99% identical to the sequence of SEQ ID NO: 15, and a light chain variable region that is at least 95%, 96%, 97%, 98%, or 99% identical to the sequence of SEQ ID NO: 16.

In still a further embodiment, the first Fab molecule under a) comprises the heavy chain variable region sequence of SEQ ID NO: 15, and the light chain variable region sequence of SEQ ID NO: 16.

In one embodiment, the second Fab molecule and the third Fab molecule under b) each comprise a heavy chain variable region that is at least 95%, 96%, 97%, 98%, or 99% identical to the sequence of SEQ ID NO: 7, and a light chain variable region that is at least 95%, 96%, 97%, 98%, or 99% identical to the sequence of SEQ ID NO: 8.

In one embodiment, the second Fab molecule under and the third Fab molecule under b) each comprise the heavy chain variable region sequence of SEQ ID NO: 7, and the light chain variable region sequence of SEQ ID NO: 8.

In a particular embodiment, the anti-CD20/anti-CD3 bispecific antibody comprises a polypeptide that is at least 95%, 96%, 97%, 98%, or 99% identical to the sequence of SEQ ID NO: 17, a polypeptide that is at least 95%, 96%, 97%, 98%, or 99% identical to the sequence of SEQ ID NO: 18, a polypeptide that is at least 95%, 96%, 97%, 98%, or 99% identical to the sequence of SEQ ID NO: 19, and a polypeptide that is at least 95%, 96%, 97%, 98%, or 99% identical to the sequence of SEQ ID NO: 20. In a further particular embodiment, the bispecific antibody comprises a polypeptide sequence of SEQ ID NO: 17, a polypeptide sequence of SEQ ID NO: 18, a polypeptide sequence of SEQ ID NO: 19 and a polypeptide sequence of SEQ ID NO: 20. In a further particular embodiment, the bispecific antibody comprises one polypeptide chain comprising SEQ ID NO: 17, one polypeptide chain comprising SEQ ID NO: 18, one polypeptide chain comprising SEQ ID NO: 19, and two polypeptide chains comprising SEQ ID NO: 20.

Particular anti-CD20/anti-CD3 bispecific antibodies are described in PCT publication no. WO 2016/020309 and European patent application nos. EP15188093 and EP16169160 (each incorporated herein by reference in its entirety).

Glofitamab

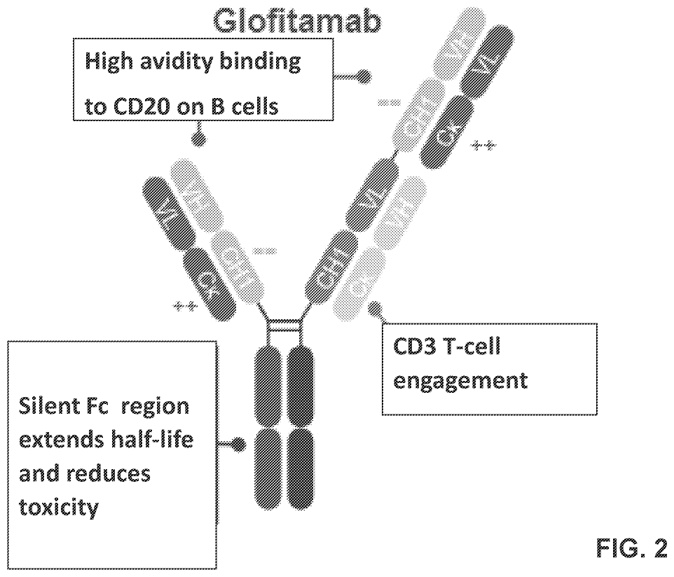

In one embodiment the anti-CD20/anti-CD3 bispecific antibody useful in the methods provided herein is glofitamab. Glofitamab (WHO Drug Information (International Nonproprietary Names for Pharmaceutical Substances), Recommended INN: List 83, 2020, vol. 34, no. 1, p. 39; Proposed INN: List 121 WHO Drug Information, Vol. 33, No. 2, 2019, page 276, also known as CD20-TCB, R07082859, or RG6026; CAS #: 2229047-91-8) is a novel T-cell-engaging bispecific (TCB) full-length antibody with a 2:1 molecular configuration for bivalent binding to CD20 on B cells and monovalent binding to CD3, particularly the CD3 epsilon chain (CD3e), on T cells. Its CD3-binding region is fused to one of the CD20-binding regions in a head-to-tail fashion via a flexible linker. This structure endows glofitamab with superior in vitro potency versus other CD20-CD3 bispecific antibodies with a 1:1 configuration and leads to profound antitumor efficacy in preclinical DLBCL models. CD20 bivalency preserves this potency in the presence of competing anti-CD20 antibodies, providing the opportunity for pre- or co-treatment with these agents. Glofitamab comprises an engineered, heterodimeric Fc region with completely abolished binding to FcgRs and C1q. By simultaneously binding to human CD20-expressing tumor cells and to the CD3e of the T-cell receptor (TCR) complex on T-cells, it induces tumor cell lysis, in addition to T-cell activation, proliferation and cytokine release. Lysis of B-cells mediated by glofitamab is CD20-specific and does not occur in the absence of CD20 expression or in the absence of simultaneous binding (cross-linking) of T-cells to CD20-expressing cells. In addition to killing, T-cells undergo activation due to CD3 cross-linking, as detected by an increase in T-cell activation markers (CD25 and CD69), cytokine release (IFNγ, TNFα, IL-2, IL-6, IL-10), cytotoxic granule release (Granzyme B) and T-cell proliferation. A schematic of the molecule structure of glofitamab is depicted in FIG. 2. The sequences of glofitamab are summarized in Table 2.

TABLE 2
Sequence IDs for glofitamab

| SEQ ID NO: | Description | SEQ ID NO: | Description |
|---|---|---|---|
| CD3 Heavy Chain | | CD3 Light Chain | |
| 9 | HVR-H1 (Kabat) | 12 | HVR-L1 (Kabat) |
| 10 | HVR-H2 (Kabat) | 13 | HVR-L2 (Kabat) |
| 11 | HVR-H3 (Kabat) | 14 | HVR-L3 (Kabat) |
| 15 | VH | 16 | VL |
| CD20 Heavy Chain | | CD20 Light Chain | |
| 1 | HVR-H1 (Kabat) | 4 | HVR-L1 (Kabat) |
| 2 | HVR-H2 (Kabat) | 5 | HVR-L2 (Kabat) |
| 3 | HVR-H3 (Kabat) | 6 | HVR-L3 (Kabat) |
| 7 | VH | 8 | VH |

Full-length antibody

| | | | |
|---|---|---|---|
| 17 | HC-knob | 18 | HC-hole |
| 19 | LC-CD3 | 20 | LC-CD20 |

### B. Antibody Formats

1. Anti-CD20/Anti-CD3 Bispecific Antibody

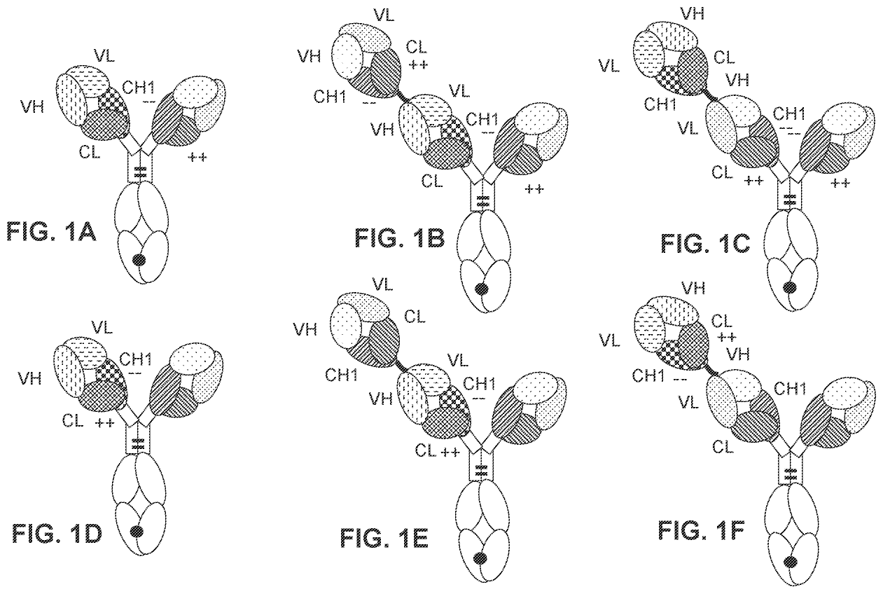

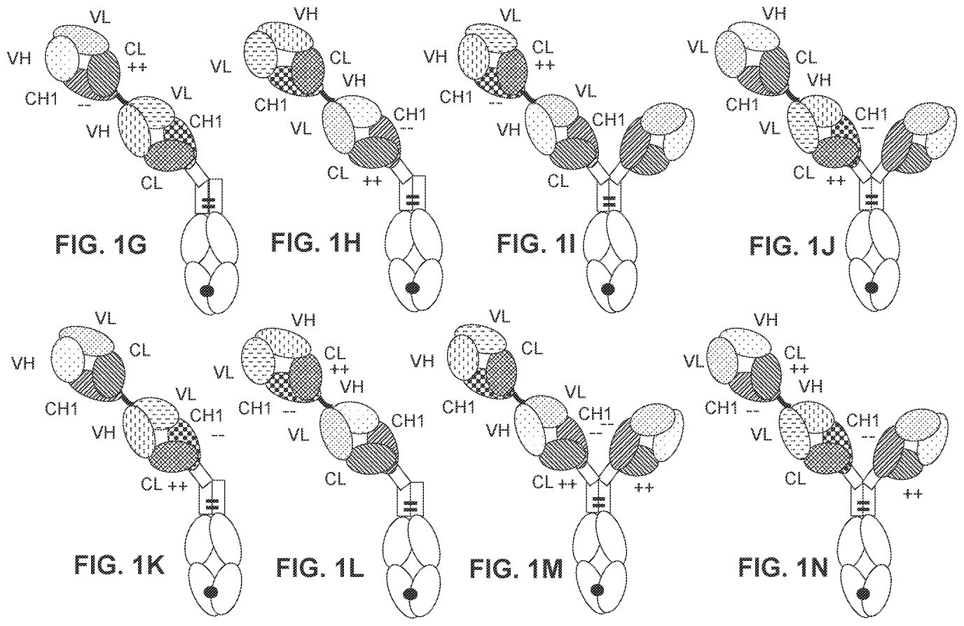

The components of the anti-CD20/anti-CD3 bispecific antibody useful in the treatment methods provided herein can be fused to each other in a variety of configurations. Exemplary configurations are depicted in FIG. 1.

In particular embodiments, the antigen binding moieties comprised in the anti-CD20/anti-CD3 bispecific antibody are Fab molecules. In such embodiments, the first, second, third, etc. antigen binding moiety may be referred to herein as first, second, third, etc. Fab molecule, respectively. Furthermore, in particular embodiments, the anti-CD20/anti-CD3 bispecific antibody comprises an Fc domain composed of a first and a second subunit capable of stable association.

In some embodiments, the first Fab molecule is fused at the C-terminus of the Fab heavy chain to the N-terminus of the first or the second subunit of the Fc domain.

In one such embodiment, the second Fab molecule is fused at the C-terminus of the Fab heavy chain to the N-terminus of the Fab heavy chain of the first Fab molecule. In a specific such embodiment, the anti-CD20/anti-CD3 bispecific antibody essentially consists of the first and the second Fab molecule, the Fc domain composed of a first and a second subunit, and optionally one or more peptide linkers, wherein the first Fab molecule is fused at the C-terminus of the Fab heavy chain to the N-terminus of the first or the second subunit of the Fc domain and the second Fab molecule is fused at the C-terminus of the Fab heavy chain to the N-terminus of the Fab heavy chain of the first Fab molecule. Such a configuration is schematically depicted in FIGS. 1G and 1K. Optionally, the Fab light chain of the first Fab molecule and the Fab light chain of the second Fab molecule may additionally be fused to each other.

In another embodiment, the second Fab molecule is fused at the C-terminus of the Fab heavy chain to the N-terminus of the first or second subunit of the Fc domain. In a specific such embodiment, the antibody essentially consists of the first and the second Fab molecule, the Fc domain composed of a first and a second subunit, and optionally one or more peptide linkers, wherein the first and the second Fab molecule are each fused at the C-terminus of the Fab heavy chain to the N-terminus of one of the subunits of the Fc domain. Such a configuration is schematically depicted in FIGS. 1A and 1D. The first and the second Fab molecule may be fused to the Fc domain directly or through a peptide linker. In a particular embodiment the first and the second Fab molecule are each fused to the Fc domain through an immunoglobulin hinge region. In a specific embodiment, the immunoglobulin hinge region is a human IgG₁ hinge region, particularly where the Fc domain is an IgG₁ Fc domain.

In other embodiments, the second Fab molecule is fused at the C-terminus of the Fab heavy chain to the N-terminus of the first or second subunit of the Fc domain. In one such embodiment, the first Fab molecule is fused at the C-terminus of the Fab heavy chain to the N-terminus of the Fab heavy chain of the second Fab molecule. In a specific such embodiment, the antibody essentially consists of the first and the second Fab molecule, the Fc domain composed of a first and a second subunit, and optionally one or more peptide linkers, wherein the first Fab molecule is fused at the C-terminus of the Fab heavy chain to the N-terminus of the Fab heavy chain of the second Fab molecule, and the second Fab molecule is fused at the C-terminus of the Fab heavy chain to the N-terminus of the first or the second subunit of the Fc domain. Such a configuration is schematically depicted in FIGS. 1H and 1L. Optionally, the Fab light chain of the first Fab molecule and the Fab light chain of the second Fab molecule may additionally be fused to each other.

The Fab molecules may be fused to the Fc domain or to each other directly or through a peptide linker, comprising one or more amino acids, typically about 2-20 amino acids. Peptide linkers are known in the art and are described herein. Suitable, non-immunogenic peptide linkers include, for example, (G₄S)ₙ (SEQ ID NO: 21), (SG₄)ₙ (SEQ ID NO: 22), or G₄(SG₄)ₙ (SEQ ID NO: 23) peptide linkers. “n” is generally an integer from 1 to 10, typically from 2 to 4. In one embodiment said peptide linker has a length of at least 5 amino acids, in one embodiment a length of 5 to 100, in a further embodiment of 10 to 50 amino acids. In one embodiment said peptide linker is (GxS)ₙ or (GxS)ₙGₘ with G=glycine, S=serine, and (x=3, n=3, 4, 5 or 6, and m=0, 1, 2 or 3) or (x=4, n=2, 3, 4 or 5 and m=0, 1, 2 or 3), in one embodiment x=4 and n=2 or 3, in a further embodiment x=4 and n=2 (SEQ ID NOs: 25-56). In one embodiment said peptide linker is (G₄S)₂ (SEQ ID NO 29). A particularly suitable peptide linker for fusing the Fab light chains of the first and the second Fab molecule to each other is (G₄S)₂ (SEQ ID NO: 29). An exemplary peptide linker suitable for connecting the Fab heavy chains of the first and the second Fab fragments comprises the sequence (D)-(G₄S)₂ (SEQ ID NOs: 29 and 57). Another suitable such linker comprises the sequence (G₄S)₄ (SEQ ID NO 24). Additionally, linkers may comprise (a portion of) an immunoglobulin hinge region. Particularly where a Fab molecule is fused to the N-terminus of an Fc domain subunit, it may be fused via an immunoglobulin hinge region or a portion thereof, with or without an additional peptide linker.

An antibody with a single antigen binding moiety (such as a Fab molecule) capable of specific binding to a target cell antigen (for example as shown in FIGS. 1A, 1D, 1G, 1H, 1K, and 1L) is useful, particularly in cases where internalization of the target cell antigen is to be expected following binding of a high affinity antigen binding moiety. In such cases, the presence of more than one antigen binding moiety specific for the target cell antigen may enhance internalization of the target cell antigen, thereby reducing its availability.

In many other cases, however, it will be advantageous to have an antibody comprising two or more antigen binding moieties (such as Fab molecules) specific for a target cell antigen (see examples shown in FIGS. 1B, 1C, 1E, 1F, 1I, 1J, 1M, and 1N), for example to optimize targeting to the target site or to allow crosslinking of target cell antigens.

Accordingly, in particular embodiments, the anti-CD20/anti-CD3 bispecific antibody comprises two anti-CD20 binding moieties, e.g., two Fab molecules targeting CD20. In one embodiment the two Fab molecules targeting CD20 are conventional Fab molecules. In one embodiment, the two Fab molecules targeting CD20 comprise the same heavy and light chain amino acid sequences and have the same arrangement of domains (i.e., conventional or crossover).

In alternative embodiments, the anti-CD20/anti-CD3 bispecific antibody comprises two anti-CD3 binding moieties, e.g., two Fab molecules targeting CD3. In one such embodiment, the two Fab molecules targeting CD3 are both crossover Fab molecules (a Fab molecule wherein the variable domains VH and VL or the constant domains CL and CH1 of the Fab heavy and light chains are exchanged/replaced by each other). In one such embodiment, the two Fab molecules targeting CD3 comprise the same heavy and light chain amino acid sequences and have the same arrangement of domains (i.e., conventional or crossover).

In one embodiment, the third Fab molecule is fused at the C-terminus of the Fab heavy chain to the N-terminus of the first or second subunit of the Fc domain.

In a particular embodiment, the second and the third Fab molecule are each fused at the C-terminus of the Fab heavy chain to the N-terminus of one of the subunits of the Fc domain, and the first Fab molecule is fused at the C-terminus of the Fab heavy chain to the N-terminus of the Fab heavy chain of the second Fab molecule. In a specific such embodiment, the antibody essentially consists of the first, the second and the third Fab molecule, the Fc domain composed of a first and a second subunit, and optionally one or more peptide linkers, wherein the first Fab molecule is fused at the C-terminus of the Fab heavy chain to the N-terminus of the Fab heavy chain of the second Fab molecule, and the second Fab molecule is fused at the C-terminus of the Fab heavy chain to the N-terminus of the first subunit of the Fc domain, and wherein the third Fab molecule is fused at the C-terminus of the Fab heavy chain to the N-terminus of the second subunit of the Fc domain. Such a configuration is schematically depicted in FIG. 1B and FIG. 1E (embodiments wherein the third Fab molecule is a conventional Fab molecule and identical to the second Fab molecule), and FIG. 1I and FIG. 1M (embodiments wherein the third Fab molecule is a crossover Fab molecule and preferably identical to the first Fab molecule). The second and the third Fab molecule may be fused to the Fc domain directly or through a peptide linker. In a particular embodiment the second and the third Fab molecule are each fused to the Fc domain through an immunoglobulin hinge region. In a specific embodiment, the immunoglobulin hinge region is a human IgG₁ hinge region, particularly where the Fc domain is an IgG₁ Fc domain. Optionally, the Fab light chain of the first Fab molecule and the Fab light chain of the second Fab molecule may additionally be fused to each other.

In another embodiment, the second and the third Fab molecule are each fused at the C-terminus of the Fab heavy chain to the N-terminus of one of the subunits of the Fc domain, and the first Fab molecule is fused at the C-terminus of the Fab heavy chain to the N-terminus of the Fab heavy chain of the second Fab molecule. In a specific such embodiment, the antibody essentially consists of the first, the second and the third Fab molecule, the Fc domain composed of a first and a second subunit, and optionally one or more peptide linkers, wherein the first Fab molecule is fused at the C-terminus of the Fab heavy chain to the N-terminus of the Fab heavy chain of the second Fab molecule, and the second Fab molecule is fused at the C-terminus of the Fab heavy chain to the N-terminus of the first subunit of the Fc domain, and wherein the third Fab molecule is fused at the C-terminus of the Fab heavy chain to the N-terminus of the second subunit of the Fc domain. Such a configuration is schematically depicted in FIG. 1C and FIG. 1F (embodiments wherein the third Fab molecule is a conventional Fab molecule and identical to the second Fab molecule) and in FIG. 1J and FIG. 1N (embodiments wherein the third Fab molecule is a crossover Fab molecule and identical to the first Fab molecule). The first and the third Fab molecule may be fused to the Fc domain directly or through a peptide linker. In a particular embodiment the second and the third Fab molecule are each fused to the Fc domain through an immunoglobulin hinge region. In a specific embodiment, the immunoglobulin hinge region is a human IgG₁ hinge region, particularly where the Fc domain is an IgG₁ Fc domain. Optionally, the Fab light chain of the first Fab molecule and the Fab light chain of the second Fab molecule may additionally be fused to each other.

In configurations of the antibody wherein a Fab molecule is fused at the C-terminus of the Fab heavy chain to the N-terminus of each of the subunits of the Fc domain through an immunoglobulin hinge regions, the two Fab molecules, the hinge regions and the Fc domain essentially form an immunoglobulin molecule. In a particular embodiment, the immunoglobulin molecule is an IgG class immunoglobulin. In an even more particular embodiment, the immunoglobulin is an IgG₁ subclass immunoglobulin. In another embodiment, the immunoglobulin is an IgG₄ subclass immunoglobulin. In a further particular embodiment, the immunoglobulin is a human immunoglobulin. In other embodiments, the immunoglobulin is a chimeric immunoglobulin or a humanized immunoglobulin.

In some of the antibodies, the Fab light chain of the first Fab molecule and the Fab light chain of the second Fab molecule are fused to each other, optionally via a peptide linker. Depending on the configuration of the first and the second Fab molecule, the Fab light chain of the first Fab molecule may be fused at its C-terminus to the N-terminus of the Fab light chain of the second Fab molecule, or the Fab light chain of the second Fab molecule may be fused at its C-terminus to the N-terminus of the Fab light chain of the first Fab molecule. Fusion of the Fab light chains of the first and the second Fab molecule further reduces mispairing of unmatched Fab heavy and light chains, and also reduces the number of plasmids needed for expression of some of the antibodies.

In certain embodiments, the antibody comprises a polypeptide wherein the Fab light chain variable region of the first Fab molecule shares a carboxy-terminal peptide bond with the Fab heavy chain constant region of the first Fab molecule (i.e., the first Fab molecule comprises a crossover Fab heavy chain, wherein the heavy chain variable region is replaced by a light chain variable region), which in turn shares a carboxy-terminal peptide bond with an Fc domain subunit (VL₍₁₎-CH1₍₁₎-CH2-CH3(-CH4)), and a polypeptide wherein the Fab heavy chain of the second Fab molecule shares a carboxy-terminal peptide bond with an Fc domain subunit (VH₍₂₎—CH1₍₂₎—CH2-CH3(-CH4)). In some embodiments, the antibody further comprises a polypeptide wherein the Fab heavy chain variable region of the first Fab molecule shares a carboxy-terminal peptide bond with the Fab light chain constant region of the first Fab molecule (VH₍₁₎-CL₍₁₎) and the Fab light chain polypeptide of the second Fab molecule (VL₍₂₎-CL₍₂₎). In certain embodiments, the polypeptides are covalently linked, e.g., by a disulfide bond.

In certain embodiments, the antibody comprises a polypeptide wherein the Fab heavy chain variable region of the first Fab molecule shares a carboxy-terminal peptide bond with the Fab light chain constant region of the first Fab molecule (i.e., the first Fab molecule comprises a crossover Fab heavy chain, wherein the heavy chain constant region is replaced by a light chain constant region), which in turn shares a carboxy-terminal peptide bond with an Fc domain subunit (VH₍₁₎-CL₍₁₎-CH2-CH3(-CH4)), and a polypeptide wherein the Fab heavy chain of the second Fab molecule shares a carboxy-terminal peptide bond with an Fc domain subunit (VH₍₂₎-CH1₍₂₎-CH2-CH3(-CH4)). In some embodiments, the antibody further comprises a polypeptide wherein the Fab light chain variable region of the first Fab molecule shares a carboxy-terminal peptide bond with the Fab heavy chain constant region of the first Fab molecule (VL₍₁₎-CH1₍₁₎) and the Fab light chain polypeptide of the second Fab molecule (VL₍₂₎-CL₍₂₎). In certain embodiments the polypeptides are covalently linked, e.g., by a disulfide bond.

In some embodiments, the antibody comprises a polypeptide wherein the Fab light chain variable region of the first Fab molecule shares a carboxy-terminal peptide bond with the Fab heavy chain constant region of the first Fab molecule (i.e., the first Fab molecule comprises a crossover Fab heavy chain, wherein the heavy chain variable region is replaced by a light chain variable region), which in turn shares a carboxy-terminal peptide bond with the Fab heavy chain of the second Fab molecule, which in turn shares a carboxy-terminal peptide bond with an Fc domain subunit (VL₍₁₎-CH1₍₁₎-VH₍₂₎-CH1₍₂₎-CH2-CH3(-CH4)). In other embodiments, the antibody comprises a polypeptide wherein the Fab heavy chain of the second Fab molecule shares a carboxy-terminal peptide bond with the Fab light chain variable region of the first Fab molecule which in turn shares a carboxy-terminal peptide bond with the Fab heavy chain constant region of the first Fab molecule (i.e., the first Fab molecule comprises a crossover Fab heavy chain, wherein the heavy chain variable region is replaced by a light chain variable region), which in turn shares a carboxy-terminal peptide bond with an Fc domain subunit (VH₍₂₎-CH1₍₂₎-V1₍₁₎-CH1₍₁₎-CH2-CH3(-CH4)).

In some of these embodiments, the antibody further comprises a crossover Fab light chain polypeptide of the first Fab molecule, wherein the Fab heavy chain variable region of the first Fab molecule shares a carboxy-terminal peptide bond with the Fab light chain constant region of the first Fab molecule (VH₍₁₎-CL₍₁₎), and the Fab light chain polypeptide of the second Fab molecule (VL₍₂₎-CL₍₂₎). In others of these embodiments, the antibody further comprises a polypeptide wherein the Fab heavy chain variable region of the first Fab molecule shares a carboxy-terminal peptide bond with the Fab light chain constant region of the first Fab molecule which in turn shares a carboxy-terminal peptide bond with the Fab light chain polypeptide of the second Fab molecule (VH₍₁₎-CL₍₁₎-VL₍₂₎-CL₍₂₎), or a polypeptide wherein the Fab light chain polypeptide of the second Fab molecule shares a carboxy-terminal peptide bond with the Fab heavy chain variable region of the first Fab molecule which in turn shares a carboxy-terminal peptide bond with the Fab light chain constant region of the first Fab molecule (VL₍₂₎-CL₍₂₎-VH₍₁₎-CL₍₁₎), as appropriate.

The antibody according to these embodiments may further comprise (i) an Fc domain subunit polypeptide (CH2-CH3(-CH4)), or (ii) a polypeptide wherein the Fab heavy chain of a third Fab molecule shares a carboxy-terminal peptide bond with an Fc domain subunit (VH₍₃₎-CH1₍₃₎-CH2-CH3(-CH4)) and the Fab light chain polypeptide of a third Fab molecule (VL₍₃₎-CL₍₃₎). In certain embodiments the polypeptides are covalently linked, e.g., by a disulfide bond.

In some embodiments, the antibody comprises a polypeptide wherein the Fab heavy chain variable region of the second Fab molecule shares a carboxy-terminal peptide bond with the Fab light chain constant region of the first Fab molecule (i.e., the first Fab molecule comprises a crossover Fab heavy chain, wherein the heavy chain constant region is replaced by a light chain constant region), which in turn shares a carboxy-terminal peptide bond with the Fab heavy chain of the second Fab molecule, which in turn shares a carboxy-terminal peptide bond with an Fc domain subunit (VH₍₁₎-CL₍₁₎-VH₍₂₎-CH1₍₂₎-CH2-CH3(-CH4)). In other embodiments, the antibody comprises a polypeptide wherein the Fab heavy chain of the second Fab molecule shares a carboxy-terminal peptide bond with the Fab heavy chain variable region of the first Fab molecule which in turn shares a carboxy-terminal peptide bond with the Fab light chain constant region of the first Fab molecule (i.e., the first Fab molecule comprises a crossover Fab heavy chain, wherein the heavy chain constant region is replaced by a light chain constant region), which in turn shares a carboxy-terminal peptide bond with an Fc domain subunit (VH₍₂₎-CH1₍₂₎-VH₍₁₎-CL(1)-CH2-CH3(-CH4)).

In some of these embodiments, the antibody further comprises a crossover Fab light chain polypeptide of the first Fab molecule, wherein the Fab light chain variable region of the first Fab molecule shares a carboxy-terminal peptide bond with the Fab heavy chain constant region of the first Fab molecule (VL₍₁₎-CH1₍₁₎), and the Fab light chain polypeptide of the second Fab molecule (VL₍₂₎-CL₍₂₎). In others of these embodiments, the antibody further comprises a polypeptide wherein the Fab light chain variable region of the first Fab molecule shares a carboxy-terminal peptide bond with the Fab heavy chain constant region of the first Fab molecule which in turn shares a carboxy-terminal peptide bond with the Fab light chain polypeptide of the second Fab molecule (VL₍₁₎-CH1₍₁₎-VL₍₂₎-CL₍₂₎), or a polypeptide wherein the Fab light chain polypeptide of the second Fab molecule shares a carboxy-terminal peptide bond with the Fab heavy chain variable region of the first Fab molecule which in turn shares a carboxy-terminal peptide bond with the Fab light chain constant region of the first Fab molecule (VL₍₂₎-CL₍₂₎-VH₍₁₎-CL₍₁₎), as appropriate.

The antibody according to these embodiments may further comprise (i) an Fc domain subunit polypeptide (CH2-CH3(-CH4)), or (ii) a polypeptide wherein the Fab heavy chain of a third Fab molecule shares a carboxy-terminal peptide bond with an Fc domain subunit (VH₍₃₎-CH1₍₃₎-CH2-CH3(-CH4)) and the Fab light chain polypeptide of a third Fab molecule (VL₍₃₎-CL₍₃₎). In certain embodiments, the polypeptides are covalently linked, e.g., by a disulfide bond.

In certain embodiments, the antibody comprises a polypeptide wherein the Fab heavy chain of the first Fab molecule shares a carboxy-terminal peptide bond with the Fab light chain variable region of the second Fab molecule, which in turn shares a carboxy-terminal peptide bond with the Fab heavy chain constant region of the second Fab molecule (i.e., the second Fab molecule comprises a crossover Fab heavy chain, wherein the heavy chain variable region is replaced by a light chain variable region) (VH₍₁₎-CH1₍₁₎-VL₍₂₎-CH1₍₂₎). In some embodiments, the antibody further comprises a polypeptide wherein the Fab heavy chain variable region of the second Fab molecule shares a carboxy-terminal peptide bond with the Fab light chain constant region of the second Fab molecule (VH₍₂₎-CL₍₂₎) and the Fab light chain polypeptide of the first Fab molecule (VL₍₁₎-CL₍₁₎).

In certain embodiments, the antibody comprises a polypeptide wherein the Fab light chain variable region of the second Fab molecule shares a carboxy-terminal peptide bond with the Fab heavy chain constant region of the second Fab molecule (i.e., the second Fab molecule comprises a crossover Fab heavy chain, wherein the heavy chain variable region is replaced by a light chain variable region), which in turn shares a carboxy-terminal peptide bond with the Fab heavy chain of the first Fab molecule (VL₍₂₎-CH1₍₂₎-VH₍₁₎-CH1₍₁₎). In some embodiments, the antibody further comprises a polypeptide wherein the Fab heavy chain variable region of the second Fab molecule shares a carboxy-terminal peptide bond with the Fab light chain constant region of the second Fab molecule (VH₍₂₎-CL₍₂₎) and the Fab light chain polypeptide of the first Fab molecule (VL₍₁₎-CL₍₁₎).

In certain embodiments, the antibody comprises a polypeptide wherein the Fab heavy chain variable region of the second Fab molecule shares a carboxy-terminal peptide bond with the Fab light chain constant region of the second Fab molecule (i.e., the second Fab molecule comprises a crossover

Fab heavy chain, wherein the heavy chain constant region is replaced by a light chain constant region), which in turn shares a carboxy-terminal peptide bond with the Fab heavy chain of the first Fab molecule (VH₍₂₎-CL₍₂₎-VH₍₁₎-CH1₍₁₎). In some embodiments, the antibody further comprises a polypeptide wherein the Fab light chain variable region of the second Fab molecule shares a carboxy-terminal peptide bond with the Fab heavy chain constant region of the second Fab molecule (VL₍₂₎-CH1₍₂₎) and the Fab light chain polypeptide of the first Fab molecule (VL₍₁₎-CL₍₁₎).

In certain embodiments, the antibody comprises a polypeptide wherein the Fab heavy chain of a third Fab molecule shares a carboxy-terminal peptide bond with the Fab heavy chain of the first Fab molecule, which in turn shares a carboxy-terminal peptide bond with the Fab light chain variable region of the second Fab molecule, which in turn shares a carboxy-terminal peptide bond with the Fab heavy chain constant region of the second Fab molecule (i.e., the second Fab molecule comprises a crossover Fab heavy chain, wherein the heavy chain variable region is replaced by a light chain variable region) (VH₍₃₎-CH1₍₃₎-VH₍₁₎-CH1₍₁₎-VL₍₂₎-CH1₍₂₎). In some embodiments, the antibody further comprises a polypeptide wherein the Fab heavy chain variable region of the second Fab molecule shares a carboxy-terminal peptide bond with the Fab light chain constant region of the second Fab molecule (VH₍₂₎-CL₍₂₎) and the Fab light chain polypeptide of the first Fab molecule (VL₍₁₎-CL₍₁₎). In some embodiments, the antibody further comprises the Fab light chain polypeptide of a third Fab molecule (VL₍₃₎-CL₍₃₎).

In certain embodiments, the antibody comprises a polypeptide wherein the Fab heavy chain of a third Fab molecule shares a carboxy-terminal peptide bond with the Fab heavy chain of the first Fab molecule, which in turn shares a carboxy-terminal peptide bond with the Fab heavy chain variable region of the second Fab molecule, which in turn shares a carboxy-terminal peptide bond with the Fab light chain constant region of the second Fab molecule (i.e., the second Fab molecule comprises a crossover Fab heavy chain, wherein the heavy chain constant region is replaced by a light chain constant region) (VH₍₃₎-CH1₍₃₎-VH₍₁₎-CH1₍₁₎-VH₍₂₎-CL₍₂₎). In some embodiments, the antibody further comprises a polypeptide wherein the Fab light chain variable region of the second Fab molecule shares a carboxy-terminal peptide bond with the Fab heavy chain constant region of the second Fab molecule (VL₍₂₎-CH1₍₂₎) and the Fab light chain polypeptide of the first Fab molecule (VL₍₁₎-CL₍₁₎). In some embodiments, the antibody further comprises the Fab light chain polypeptide of a third Fab molecule (VL₍₃₎-CL₍₃₎).

In certain embodiments, the antibody comprises a polypeptide wherein the Fab light chain variable region of the second Fab molecule shares a carboxy-terminal peptide bond with the Fab heavy chain constant region of the second Fab molecule (i.e., the second Fab molecule comprises a crossover Fab heavy chain, wherein the heavy chain variable region is replaced by a light chain variable region), which in turn shares a carboxy-terminal peptide bond with the Fab heavy chain of the first Fab molecule, which in turn shares a carboxy-terminal peptide bond with the Fab heavy chain of a third Fab molecule (VL₍₂₎-CH1₍₂₎-VH₍₁₎-CH1₍₁₎-VH₍₃₎-CH1₍₃₎). In some embodiments, the antibody further comprises a polypeptide wherein the Fab heavy chain variable region of the second Fab molecule shares a carboxy-terminal peptide bond with the Fab light chain constant region of the second Fab molecule (VH₍₂₎-CL₍₂₎) and the Fab light chain polypeptide of the first Fab molecule (VL₍₁₎-CL₍₁₎). In some embodiments, the antibody further comprises the Fab light chain polypeptide of a third Fab molecule (VL₍₃₎-CL₍₃₎).

In certain embodiments, the antibody comprises a polypeptide wherein the Fab heavy chain variable region of the second Fab molecule shares a carboxy-terminal peptide bond with the Fab light chain constant region of the second Fab molecule (i.e., the second Fab molecule comprises a crossover Fab heavy chain, wherein the heavy chain constant region is replaced by a light chain constant region), which in turn shares a carboxy-terminal peptide bond with the Fab heavy chain of the first Fab molecule, which in turn shares a carboxy-terminal peptide bond with the Fab heavy chain of a third Fab molecule (VH₍₂₎-CL₍₂₎-VH₍₁₎-CH1₍₁₎-VH₍₃₎-CH1₍₃₎). In some embodiments, the antibody further comprises a polypeptide wherein the Fab light chain variable region of the second Fab molecule shares a carboxy-terminal peptide bond with the Fab heavy chain constant region of the second Fab molecule (VL₍₂₎-CH1₍₂₎) and the Fab light chain polypeptide of the first Fab molecule (VL₍₁₎-CL₍₁₎). In some embodiments, the antibody further comprises the Fab light chain polypeptide of a third Fab molecule (VL₍₃₎-CL₍₃₎).

In certain embodiments, the antibody comprises a polypeptide wherein the Fab heavy chain of the first Fab molecule shares a carboxy-terminal peptide bond with the Fab light chain variable region of the second Fab molecule, which in turn shares a carboxy-terminal peptide bond with the Fab heavy chain constant region of the second Fab molecule (i.e., the second Fab molecule comprises a crossover Fab heavy chain, wherein the heavy chain variable region is replaced by a light chain variable region), which in turn shares a carboxy-terminal peptide bond with the Fab light chain variable region of a third Fab molecule, which in turn shares a carboxy-terminal peptide bond with the Fab heavy chain constant region of a third Fab molecule (i.e., the third Fab molecule comprises a crossover Fab heavy chain, wherein the heavy chain variable region is replaced by a light chain variable region) (VH₍₁₎-CH1₍₁₎-VL₍₂₎-CH1₍₂₎-VL₍₃₎-CH1₍₃₎). In some embodiments, the antibody further comprises a polypeptide wherein the Fab heavy chain variable region of the second Fab molecule shares a carboxy-terminal peptide bond with the Fab light chain constant region of the second Fab molecule (VH₍₂₎-CL₍₂₎) and the Fab light chain polypeptide of the first Fab molecule (VL₍₁₎-CL₍₁₎). In some embodiments, the antibody further comprises a polypeptide wherein the Fab heavy chain variable region of a third Fab molecule shares a carboxy-terminal peptide bond with the Fab light chain constant region of a third Fab molecule (VH₍₃₎-CL₍₃₎).

In certain embodiments, the antibody comprises a polypeptide wherein the Fab heavy chain of the first Fab molecule shares a carboxy-terminal peptide bond with the Fab heavy chain variable region of the second Fab molecule, which in turn shares a carboxy-terminal peptide bond with the Fab light chain constant region of the second Fab molecule (i.e., the second Fab molecule comprises a crossover Fab heavy chain, wherein the heavy chain constant region is replaced by a light chain constant region), which in turn shares a carboxy-terminal peptide bond with the Fab heavy chain variable region of a third Fab molecule, which in turn shares a carboxy-terminal peptide bond with the Fab light chain constant region of a third Fab molecule (i.e., the third Fab molecule comprises a crossover Fab heavy chain, wherein the heavy chain constant region is replaced by a light chain constant region) (VH₍₁₎-CH1₍₁₎-VH₍₂₎-CL₍₂₎-VH₍₃₎-CL₍₃₎). In some embodiments, the antibody further comprises a polypeptide wherein the Fab light chain variable region of the second Fab molecule shares a carboxy-terminal peptide bond with the Fab heavy chain constant region of the second Fab molecule (VL₍₂₎-CH1₍₂₎) and the Fab light chain polypeptide of the first Fab molecule (VL₍₁₎-CL₍₁₎). In some embodiments, the antibody further comprises a polypeptide wherein the Fab light chain variable region of a third Fab molecule shares a carboxy-terminal peptide bond with the Fab heavy chain constant region of a third Fab molecule (VL₍₃₎-CH1₍₃₎).

In certain embodiments, the antibody comprises a polypeptide wherein the Fab light chain variable region of a third Fab molecule shares a carboxy-terminal peptide bond with the Fab heavy chain constant region of a third Fab molecule (i.e., the third Fab molecule comprises a crossover Fab heavy chain, wherein the heavy chain variable region is replaced by a light chain variable region), which in turn shares a carboxy-terminal peptide bond with the Fab light chain variable region of the second Fab molecule, which in turn shares a carboxy-terminal peptide bond with the Fab heavy chain constant region of the second Fab molecule (i.e., the second Fab molecule comprises a crossover Fab heavy chain, wherein the heavy chain variable region is replaced by a light chain variable region), which in turn shares a carboxy-terminal peptide bond with the Fab heavy chain of the first Fab molecule (VL₍₃₎-CH1₍₃₎-VL₍₂₎-CH1₍₂₎-VH₍₁₎-CH1₍₁₎). In some embodiments, the antibody further comprises a polypeptide wherein the Fab heavy chain variable region of the second Fab molecule shares a carboxy-terminal peptide bond with the Fab light chain constant region of the second Fab molecule (VH₍₂₎-CL₍₂₎) and the Fab light chain polypeptide of the first Fab molecule (VL₍₁₎-CL₍₁₎). In some embodiments, the antibody further comprises a polypeptide wherein the Fab heavy chain variable region of a third Fab molecule shares a carboxy-terminal peptide bond with the Fab light chain constant region of a third Fab molecule (VH₍₃₎-CL₍₃₎).

In certain embodiments, the antibody comprises a polypeptide wherein the Fab heavy chain variable region of a third Fab molecule shares a carboxy-terminal peptide bond with the Fab light chain constant region of a third Fab molecule (i.e., the third Fab molecule comprises a crossover Fab heavy chain, wherein the heavy chain constant region is replaced by a light chain constant region), which in turn shares a carboxy-terminal peptide bond with the Fab heavy chain variable region of the second Fab molecule, which in turn shares a carboxy-terminal peptide bond with the Fab light chain constant region of the second Fab molecule (i.e., the second Fab molecule comprises a crossover Fab heavy chain, wherein the heavy chain constant region is replaced by a light chain constant region), which in turn shares a carboxy-terminal peptide bond with the Fab heavy chain of the first Fab molecule (VH₍₃₎-CL₍₃₎-VH₍₂₎-CL₍₂₎-VH₍₁₎-CH1₍₁₎). In some embodiments, the antibody further comprises a polypeptide wherein the Fab light chain variable region of the second Fab molecule shares a carboxy-terminal peptide bond with the Fab heavy chain constant region of the second Fab molecule (VL₍₂₎-CH1₍₂₎) and the Fab light chain polypeptide of the first Fab molecule (V₍₁₎-CL₍₁₎). In some embodiments, the antibody further comprises a polypeptide wherein the Fab light chain variable region of a third Fab molecule shares a carboxy-terminal peptide bond with the Fab heavy chain constant region of a third Fab molecule (VL₍₃₎-CH1₍₃₎).

According to any of the above embodiments, components of the antibody (e.g., Fab molecules, Fc domain) may be fused directly or through various linkers, particularly peptide linkers comprising one or more amino acids, typically about 2-20 amino acids, that are described herein or are known in the art. Suitable, non-immunogenic peptide linkers include, for example, (G₄S)ₙ (SEQ ID NO 21), (SG₄)ₙ (SEQ ID NO 22), or G₄(SG₄)ₙ (SEQ ID NO 23) peptide linkers, wherein n is generally an integer from 1 to 10, typically from 2 to 4.

2. Fc Domain

The anti-CD20/anti-CD3 bispecific antibody useful in the treatment methods provided herein may comprise an Fc domain which consists of a pair of polypeptide chains comprising heavy chain domains of an antibody molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other.

In one embodiment, the Fc domain is an IgG Fc domain. In a particular embodiment, the Fc domain is an IgG1 Fc domain. In another embodiment the Fc domain is an IgG₄ Fc domain. In a more specific embodiment, the Fc domain is an IgG₄ Fc domain comprising an amino acid substitution at position S228 (Kabat numbering), particularly the amino acid substitution S228P. This amino acid substitution reduces in vivo Fab arm exchange of IgG₄ antibodies (see Stubenrauch et al., Drug Metabolism and Disposition 38, 84-91 (2010)). In a further particular embodiment, the Fc domain is human.

### (i) Fc Domain Modifications Promoting Heterodimerization

The anti-CD20/anti-CD3 bispecific antibody useful in the treatment methods provided herein may comprise different components (e.g., antigen binding domains) fused to one or the other of the two subunits of the Fc domain, thus the two subunits of the Fc domain are typically comprised in two non-identical polypeptide chains. Recombinant co-expression of these polypeptides and subsequent dimerization leads to several possible combinations of the two polypeptides. To improve the yield and purity of such antibodies in recombinant production, it will thus be advantageous to introduce in the Fc domain of the antibody a modification promoting the association of the desired polypeptides.

Accordingly, in particular embodiments the Fc domain comprises a modification promoting the association of the first and the second subunit of the Fc domain. The site of most extensive protein-protein interaction between the two subunits of a human IgG Fc domain is in the CH3 domain of the Fc domain. Thus, in one embodiment said modification is in the CH3 domain of the Fc domain.

Several approaches for modifications in the CH3 domain of the Fc domain in order to enforce heterodimerization are well described, e.g., in WO 96/27011, WO 98/050431, EP 1870459, WO 2007/110205, WO 2007/147901, WO 2009/089004, WO 2010/129304, WO 2011/90754, WO 2011/143545, WO 2012058768, WO 2013157954, WO 2013096291. Typically, in all such approaches the CH3 domain of the first subunit of the Fc domain and the CH3 domain of the second subunit of the Fc domain are both engineered in a complementary manner so that each CH3 domain (or the heavy chain comprising it) can no longer homodimerize with itself but is forced to heterodimerize with the complementarily engineered other CH3 domain (so that the first and second CH3 domain heterodimerize and no homodimers between the two first or the two second CH3 domains are formed). These different approaches for improved heavy chain heterodimerization are contemplated as different alternatives in combination with heavy-light chain modifications (e.g., variable or constant region exchange/replacement in Fab arms, or introduction of substitutions of charged amino acids with opposite charges in the CH1/CL interface) which reduce light chain mispairing and Bence Jones-type side products.

In a specific embodiment said modification promoting the association of the first and the second subunit of the Fc domain is a so-called “knob-into-hole” modification, comprising a “knob” modification in one of the two subunits of the Fc domain and a “hole” modification in the other one of the two subunits of the Fc domain.

The knob-into-hole technology is described e.g., in U.S. Pat. Nos. 5,731,168; 7,695,936; Ridgway et al., Prot Eng. 9, 617-621 (1996) and Carter, J Immunol Meth. 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine).

Accordingly, in a particular embodiment, in the CH3 domain of the first subunit of the Fc domain an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

Preferably said amino acid residue having a larger side chain volume is selected from the group consisting of arginine (R), phenylalanine (F), tyrosine (Y), and tryptophan (W).

Preferably said amino acid residue having a smaller side chain volume is selected from the group consisting of alanine (A), serine (S), threonine (T), and valine (V).

The protuberance and cavity can be made by altering the nucleic acid encoding the polypeptides, e.g., by site-specific mutagenesis, or by peptide synthesis.

In a specific embodiment, in the CH3 domain of the first subunit of the Fc domain (the “knob” subunit) the threonine residue at position 366 is replaced with a tryptophan residue (T366W), and in the CH3 domain of the second subunit of the Fc domain (the “hole” subunit) the tyrosine residue at position 407 is replaced with a valine residue (Y407V) (EU numbering). In one embodiment, in the second subunit of the Fc domain additionally the threonine residue at position 366 is replaced with a serine residue (T366S) and the leucine residue at position 368 is replaced with an alanine residue (L368A) (EU numbering).

In yet a further embodiment, in the first subunit of the Fc domain additionally the serine residue at position 354 is replaced with a cysteine residue (S354C) or the glutamic acid residue at position 356 is replaced with a cysteine residue (E356C), and in the second subunit of the Fc domain additionally the tyrosine residue at position 349 is replaced by a cysteine residue (Y349C) (EU numbering). Introduction of these two cysteine residues results in formation of a disulfide bridge between the two subunits of the Fc domain, further stabilizing the dimer (Carter, J Immunol Methods 248, 7-15 (2001)).

In a particular embodiment, the first subunit of the Fc domain comprises amino acid substitutions S354C and T366W, and the second subunit of the Fc domain comprises amino acid substitutions Y349C, T366S, L368A and Y407V (EU numbering).

In a particular embodiment, the CD3 antigen binding moiety described herein is fused to the first subunit of the Fc domain (comprising the “knob” modification). Without wishing to be bound by theory, fusion of the CD3 antigen binding moiety to the knob-containing subunit of the Fc domain will (further) minimize the generation of bispecific antibodies comprising two CD3 antigen binding moieties (steric clash of two knob-containing polypeptides).

Other techniques of CH3-modification for enforcing the heterodimerization are contemplated as alternatives according to the invention and are described, e.g., in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954, WO 2013/096291.

In one embodiment, the heterodimerization approach described in EP 1870459 A1, is used alternatively. This approach is based on the introduction of charged amino acids with opposite charges at specific amino acid positions in the CH3/CH3 domain interface between the two subunits of the Fc domain. One preferred embodiment is amino acid mutations R409D and K370E in one of the two CH3 domains (of the Fc domain) and amino acid mutations D399K and E357K in the other one of the CH3 domains of the Fc domain (EU numbering).

In another embodiment, the anti-CD20/anti-CD3 bispecific antibody may comprise amino acid mutation T366W in the CH3 domain of the first subunit of the Fc domain and amino acid mutations T366S, L368A, and Y407V in the CH3 domain of the second subunit of the Fc domain, and additionally amino acid mutations R409D and K370E in the CH3 domain of the first subunit of the Fc domain and amino acid mutations D399K and E357K in the CH3 domain of the second subunit of the Fc domain (EU numbering).

In another embodiment, the anti-CD20/anti-CD3 bispecific antibody comprise amino acid mutations S354C and T366W in the CH3 domain of the first subunit of the Fc domain and amino acid mutations Y3490, T366S, L368A, and Y407V in the CH3 domain of the second subunit of the Fc domain, or the antibody comprises amino acid mutations Y349C and T366W in the CH3 domain of the first subunit of the Fc domain and amino acid mutations S354C, T366S, L368A, and Y407V in the CH3 domains of the second subunit of the Fc domain and additionally amino acid mutations R409D and K370E in the CH3 domain of the first subunit of the Fc domain and amino acid mutations D399K and E357K in the CH3 domain of the second subunit of the Fc domain (all EU numbering).

In one embodiment, the heterodimerization approach described in WO 2013/157953 is used alternatively. In one embodiment a first CH3 domain comprises amino acid mutation T366K and a second CH3 domain comprises amino acid mutation L351 D (EU numbering). In a further embodiment, the first CH3 domain comprises amino acid mutation L351 K (EU numbering). In a further embodiment, the second CH3 domain comprises further an amino acid mutation selected from Y349E, Y349D, and L368E (preferably L368E) (EU numbering).

In one embodiment, the heterodimerization approach described in WO 2012/058768 is used alternatively. In one embodiment, a first CH3 domain comprises amino acid mutations L351Y, Y407A, and a second CH3 domain comprises amino acid mutations T366A and K409F (EU numbering). In a further embodiment, the second CH3 domain comprises a further amino acid mutation at position T411, D399, S400, F405, N390, or K392, e.g., selected from a) T411N, T411R, T411Q, T411K, T411D, T411E, or T411W; b) D399R, D399W, D399Y, or D399K; c) S400E, S400D, S400R, or S400K; d) F4051, F405M, F405T, F405S, F405V, or F405W; e) N390R, N390K, or N390D; or f) K392V, K392M, K392R, K392L, K392F, or K392E (EU numbering). In a further embodiment, a first CH3 domain comprises amino acid mutations L351Y and Y407A and a second CH3 domain comprises amino acid mutations T366V and K409F (EU numbering). In a further embodiment, a first CH3 domain comprises amino acid mutation Y407A and a second CH3 domain comprises amino acid mutations T366A and K409F (EU numbering). In a further embodiment, the second CH3 domain further comprises amino acid mutations K392E, T411E, D399R, and S400R (EU numbering).

In one embodiment, the heterodimerization approach described in WO 2011/143545 is used alternatively, e.g., with the amino acid modification at a position selected from the group consisting of 368 and 409 (EU numbering).

In one embodiment, the heterodimerization approach described in WO 2011/090762, which also uses the knobs-into-holes technology described above, is used alternatively. In one embodiment, a first CH3 domain comprises amino acid mutation T366W and a second CH3 domain comprises amino acid mutation Y407A (EU numbering). In one embodiment, a first CH3 domain comprises amino acid mutation T366Y and a second CH3 domain comprises amino acid mutation Y407T (EU numbering).

In one embodiment, the anti-CD20/anti-CD3 bispecific antibody or the Fc domain of the anti-CD20/anti-CD3 bispecific antibody is of IgG2 subclass and the heterodimerization approach described in WO 2010/129304 is used.

In an alternative embodiment, a modification promoting association of the first and the second subunit of the Fc domain comprises a modification mediating electrostatic steering effects, e.g., as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable. In one such embodiment, a first CH3 domain comprises amino acid substitution of K392 or N392 with a negatively charged amino acid (e.g., glutamic acid (E), or aspartic acid (D), preferably K392D or N392D) and a second CH3 domain comprises amino acid substitution of D399, E356, D356, or E357 with a positively charged amino acid (e.g., lysine (K) or arginine (R), preferably D399K, E356K, D356K, or E357K, and more preferably D399K and E356K). In a further embodiment the first CH3 domain further comprises amino acid substitution of K409 or R409 with a negatively charged amino acid (e.g., glutamic acid (E), or aspartic acid (D), preferably K409D or R409D). In a further embodiment the first CH3 domain further or alternatively comprises amino acid substitution of K439 and/or K370 with a negatively charged amino acid (e.g., glutamic acid (E), or aspartic acid (D)) (EU numbering).

In yet a further embodiment, the heterodimerization approach described in WO 2007/147901 is used alternatively. In one embodiment, a first CH3 domain comprises amino acid mutations K253E, D282K, and K322D and a second CH3 domain comprises amino acid mutations D239K, E240K, and K292D (EU numbering).

In still another embodiment, the heterodimerization approach described in WO 2007/110205 can be used.

In one embodiment, the first subunit of the Fc domain comprises amino acid substitutions K392D and K409D, and the second subunit of the Fc domain comprises amino acid substitutions D356K and D399K (EU numbering).

(ii) Fc Domain Modifications Reducing Fc Receptor Binding and/or Effector Function

The Fc domain confers to an antibody, such as an anti-CD20/anti-CD3 bispecific, favorable pharmacokinetic properties, including a long serum half-life which contributes to good accumulation in the target tissue and a favorable tissue-blood distribution ratio. At the same time it may, however, lead to undesirable targeting of the antibody to cells expressing Fc receptors rather than to the preferred antigen-bearing cells. Moreover, the co-activation of Fc receptor signaling pathways may lead to cytokine release which, in combination with other immunostimulatory properties the antibody may have and the long half-life of the antibody, results in excessive activation of cytokine receptors and severe side effects upon systemic administration.

Accordingly, in particular embodiments, the Fc domain of the anti-CD20/anti-CD3 bispecific antibody exhibits reduced binding affinity to an Fc receptor and/or reduced effector function, as compared to a native IgG1 Fc domain. In one such embodiment, the Fc domain (or the molecule, e.g., antibody, comprising said Fc domain) exhibits less than 50%, preferably less than 20%, more preferably less than 10% and most preferably less than 5% of the binding affinity to an Fc receptor, as compared to a native IgG1 Fc domain (or a corresponding molecule comprising a native IgG1 Fc domain), and/or less than 50%, preferably less than 20%, more preferably less than 10% and most preferably less than 5% of the effector function, as compared to a native IgG1 Fc domain (or a corresponding molecule comprising a native IgG1 Fc domain). In one embodiment, the Fc domain (or the molecule, e.g., antibody, comprising said Fc domain) does not substantially bind to an Fc receptor and/or induce effector function. In a particular embodiment, the Fc receptor is an Fcγ receptor. In one embodiment the Fc receptor is a human Fc receptor. In one embodiment, the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment, the effector function is one or more selected from the group of CDC, ADCC, ADCP, and cytokine secretion. In a particular embodiment, the effector function is ADCC. In one embodiment the Fc domain exhibits substantially similar binding affinity to neonatal Fc receptor (FcRn), as compared to a native IgG₁ Fc domain. Substantially similar binding to FcRn is achieved when the Fc domain (or the molecule, e.g., antibody, comprising said Fc domain) exhibits greater than about 70%, particularly greater than about 80%, more particularly greater than about 90% of the binding affinity of a native IgG₁ Fc domain (or the corresponding molecule comprising a native IgG₁ Fc domain) to FcRn.

In certain embodiments, the Fc domain is engineered to have reduced binding affinity to an Fc receptor and/or reduced effector function, as compared to a non-engineered Fc domain. In particular embodiments, the Fc domain comprises one or more amino acid mutation that reduces the binding affinity of the Fc domain to an Fc receptor and/or effector function. Typically, the same one or more amino acid mutation is present in each of the two subunits of the Fc domain. In one embodiment, the amino acid mutation reduces the binding affinity of the Fc domain to an Fc receptor. In one embodiment, the amino acid mutation reduces the binding affinity of the Fc domain to an Fc receptor by at least 2-fold, at least 5-fold, or at least 10-fold. In embodiments where there is more than one amino acid mutation that reduces the binding affinity of the Fc domain to the Fc receptor, the combination of these amino acid mutations may reduce the binding affinity of the Fc domain to an Fc receptor by at least 10-fold, at least 20-fold, or even at least 50-fold. In one embodiment, the molecule, e.g., antibody, comprising an engineered Fc domain exhibits less than 20%, particularly less than 10%, more particularly less than 5% of the binding affinity to an Fc receptor as compared to a corresponding molecule comprising a non-engineered Fc domain. In a particular embodiment, the Fc receptor is an Fcγ receptor. In some embodiments, the Fc receptor is a human Fc receptor. In some embodiments, the Fc receptor is an activating Fc receptor. In a specific embodiment, the Fc receptor is an activating human Fcγ receptor, more specifically human FcγRIIIa, FcγRI or FcγRIIa, most specifically human FcγRIIIa. Preferably, binding to each of these receptors is reduced. In some embodiments, binding affinity to a complement component, specifically binding affinity to C1q, is also reduced. In one embodiment, binding affinity to neonatal Fc receptor (FcRn) is not reduced. Substantially similar binding to FcRn, i.e., preservation of the binding affinity of the Fc domain to said receptor, is achieved when the Fc domain (or the molecule, e.g., antibody, comprising said Fc domain) exhibits greater than about 70% of the binding affinity of a non-engineered form of the Fc domain (or a corresponding molecule comprising said non-engineered form of the Fc domain) to FcRn. The Fc domain, or molecule (e.g., antibody) comprising said Fc domain, may exhibit greater than about 80% and even greater than about 90% of such affinity. In certain embodiments, the Fc domain is engineered to have reduced effector function, as compared to a non-engineered Fc domain. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced crosslinking of target-bound antibodies, reduced dendritic cell maturation, or reduced T cell priming. In one embodiment, the reduced effector function is one or more selected from the group of reduced CDC, reduced ADCC, reduced ADCP, and reduced cytokine secretion. In a particular embodiment the reduced effector function is reduced ADCC. In one embodiment the reduced ADCC is less than 20% of the ADCC induced by a non-engineered Fc domain (or a corresponding molecule comprising a non-engineered Fc domain).

In one embodiment, the amino acid mutation that reduces the binding affinity of the Fc domain to an Fc receptor and/or effector function is an amino acid substitution. In one embodiment the Fc domain comprises an amino acid substitution at a position selected from the group of E233, L234, L235, N297, P331, and P329 (EU numbering). In a more specific embodiment, the Fc domain comprises an amino acid substitution at a position selected from the group of L234, L235, and P329 (EU numbering). In some embodiments, the Fc domain comprises the amino acid substitutions L234A and L235A (EU numbering). In one such embodiment, the Fc domain is an IgG₁ Fc domain, particularly a human IgG₁ Fc domain. In one embodiment, the Fc domain comprises an amino acid substitution at position P329. In a more specific embodiment, the amino acid substitution is P329A or P329G, particularly P329G (EU numbering). In one embodiment, the Fc domain comprises an amino acid substitution at position P329 and a further amino acid substitution at a position selected from E233, L234, L235, N297, and P331 (EU numbering). In a more specific embodiment, the further amino acid substitution is E233P, L234A, L235A, L235E, N297A, N297D, or P331S. In particular embodiments, the Fc domain comprises amino acid substitutions at positions P329, L234, and L235 (EU numbering). In more particular embodiments, the Fc domain comprises the amino acid mutations L234A, L235A, and P329G (“P329G LALA”). In one such embodiment, the Fc domain is an IgG₁ Fc domain, particularly a human IgG₁ Fc domain. The “P329G LALA” combination of amino acid substitutions almost completely abolishes Fcγ receptor (as well as complement) binding of a human IgG₁ Fc domain, as described in PCT publication no. WO 2012/130831, incorporated herein by reference in its entirety. WO 2012/130831 also describes methods of preparing such mutant Fc domains and methods for determining its properties such as Fc receptor binding or effector functions.

IgG₄ antibodies exhibit reduced binding affinity to Fc receptors and reduced effector functions as compared to IgG₁ antibodies. Hence, in some embodiments the Fc domain is an IgG₄ Fc domain, particularly a human IgG₄ Fc domain. In one embodiment, the IgG₄ Fc domain comprises amino acid substitutions at position S228, specifically the amino acid substitution S228P (EU numbering). To further reduce its binding affinity to an Fc receptor and/or its effector function, in one embodiment the IgG₄ Fc domain comprises an amino acid substitution at position L235, specifically the amino acid substitution L235E (EU numbering). In another embodiment, the IgG₄ Fc domain comprises an amino acid substitution at position P329, specifically the amino acid substitution P329G (EU numbering). In a particular embodiment, the IgG₄ Fc domain comprises amino acid substitutions at positions S228, L235, and P329, specifically amino acid substitutions S228P, L235E, and P329G (EU numbering). Such IgG₄ Fc domain mutants and their Fcγ receptor binding properties are described in PCT publication no. WO 2012/130831, incorporated herein by reference in its entirety.

In a particular embodiment, the Fc domain exhibiting reduced binding affinity to an Fc receptor and/or reduced effector function, as compared to a native IgG₁ Fc domain, is a human IgG₁ Fc domain comprising the amino acid substitutions L234A, L235A, and optionally P329G, or a human IgG₄ Fc domain comprising the amino acid substitutions S228P, L235E, and optionally P329G (EU numbering).

In certain embodiments, N-glycosylation of the Fc domain has been eliminated. In one such embodiment, the Fc domain comprises an amino acid mutation at position N297, particularly an amino acid substitution replacing asparagine by alanine (N297A) or aspartic acid (N297D) or glycine (N297G) (EU numbering).

In addition to the Fc domains described hereinabove and in PCT publication no. WO 2012/130831, Fc domains with reduced Fc receptor binding and/or effector function also include those with substitution of one or more of Fc domain residues 238, 265, 269, 270, 297, 327, and 329 (U.S. Pat. No. 6,737,056) (EU numbering). Such Fc mutants include Fc mutants with substitutions at two or more of amino acid positions 265, 269, 270, 297, and 327, including the so-called “DANA” Fc mutant with substitution of residues 265 and 297 to alanine (U.S. Pat. No. 7,332,581).

Mutant Fc domains can be prepared by amino acid deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.

Binding to Fc receptors can be easily determined, e.g., by ELISA, or by Surface Plasmon Resonance (SPR) using standard instrumentation such as a BIAcore instrument (GE Healthcare), and Fc receptors such as may be obtained by recombinant expression. Alternatively, binding affinity of Fc domains or molecules comprising an Fc domain for Fc receptors may be evaluated using cell lines known to express particular Fc receptors, such as human NK cells expressing FcγIIIa receptor.

Effector function of an Fc domain, or a molecule (e.g., an antibody) comprising an Fc domain, can be measured by methods known in the art. A suitable assay for measuring ADCC is described herein. Other examples of in vitro assays to assess ADCC activity of a molecule of interest are described in U.S. Pat. No. 5,500,362; Hellstrom et al. Proc Natl Acad Sci USA. 83, 7059-7063 (1986) and Hellstrom et al., Proc Natl Acad Sci USA. 82, 1499-1502 (1985); U.S. Pat. No. 5,821,337; Bruggemann et al., J Exp Med 166, 1351-1361 (1987). Alternatively, non-radioactive assays methods may be employed (see, for example, ACTI™ non-radioactive cytotoxicity assay for flow cytometry (CellTechnology, Inc. Mountain View, CA); and CYTOTOX 96® non-radioactive cytotoxicity assay (Promega, Madison, WI)). Useful effector cells for such assays include peripheral blood mononuclear cells (PBMC) and Natural Killer (NK) cells. Alternatively, or additionally, ADCC activity of the molecule of interest may be assessed i, e.g., in a animal model such as that disclosed in Clynes et al., Proc Natl Acad Sci USA 95, 652-656 (1998).

In some embodiments, binding of the Fc domain to a complement component, specifically to C1q, is reduced. Accordingly, in some embodiments wherein the Fc domain is engineered to have reduced effector function, said reduced effector function includes reduced CDC. C1q binding assays may be carried out to determine whether the Fc domain, or molecule (e.g., antibody) comprising the Fc domain, is able to bind C1q and hence has CDC activity. See e.g., C1q and C3c binding ELISA in WO 2006/029879 and WO 2005/100402. To assess complement activation, a CDC assay may be performed (see, for example, Gazzano-Santoro et al., J Immunol Methods 202, 163 (1996); Cragg et al., Blood 101, 1045-1052 (2003); and Cragg and Glennie, Blood 103, 2738-2743 (2004)).

3. Substitution, Insertion, and Deletion

In certain instances, anti-CD20/anti-CD3 bispecific antibody variants having one or more amino acid substitutions are provided for the treatment methods provided herein. Sites of interest for substitutional mutagenesis include the HVRs and FRs. Conservative substitutions are shown in Table 3 under the heading of “preferred substitutions.” More substantial changes are provided in Table 3 under the heading of “exemplary substitutions,” and as further described below in reference to amino acid side chain classes. Amino acid substitutions may be introduced into an antibody of interest and the products screened for a desired activity, for example, retained/improved antigen binding, decreased immunogenicity, or improved ADCC or CDC.

TABLE 3
Exemplary and Preferred Amino Acid Substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g., binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al., in Methods in Molecular Biology 178:1-37 (O'Brien et al., ed., Human Press, Totowa, NJ, (2001)). In some instances of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain instances, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain instances of the variant VH and VL sequences provided above, each HVR either is unaltered, or includes no more than one, two, or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

4. Glycosylation

In certain instances, anti-CD20/anti-CD3 bispecific antibodies of the invention can be altered to increase or decrease the extent to which the antibody is glycosylated. Addition or deletion of glycosylation sites to anti-CD20/anti-CD3 bispecific antibodies of the invention may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al., TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some instances, modifications of the oligosaccharide in an antibody of the invention are made in order to create antibody variants with certain improved properties.

In one instance, anti-CD20/anti-CD3 bispecific antibody variants are provided having a carbohydrate structure that lacks fucose attached (directly or indirectly) to an Fc region. For example, the amount of fucose in such antibody may be from 1% to 80%, from 1% to 65%, from 5% to 65%, or from 20% to 40%. The amount of fucose is determined by calculating the average amount of fucose within the sugar chain at Asn297, relative to the sum of all glycostructures attached to Asn297 (e. g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., U.S. Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO 2005/053742; WO 2002/031140; Okazaki et al., J. Mol. Biol. 336:1239-1249 (2004); Yamane-Ohnuki et al., Biotech. Bioeng. 87: 614 (2004). Examples of cell lines capable of producing defucosylated antibodies include Lec13 CHO cells deficient in protein fucosylation (Ripka et al., Arch. Biochem. Biophys. 249:533-545 (1986); U.S. Patent Application No. US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al., Biotech. Bioeng. 87: 614 (2004); Kanda, Y. et al., Biotechnol. Bioeng., 94(4):680-688 (2006); and WO 2003/085107).

In view of the above, in some instances, the methods of the invention involve administering to the subject in the context of a fractionated, dose-escalation dosing regimen an anti-CD20/anti-CD3 bispecific antibody variant that comprises an aglycosylation site mutation. In some instances, the aglycosylation site mutation reduces effector function of the antibody. In some instances, the aglycosylation site mutation is a substitution mutation. In some instances, the antibody comprises a substitution mutation in the Fc region that reduces effector function. In some instances, the substitution mutation is at amino acid residue N297, L234, L235, and/or D265 (EU numbering). In some instances, the substitution mutation is selected from the group consisting of N297G, N297A, L234A, L235A, D265A, and P329G. In some instances, the substitution mutation is at amino acid residue N297. In a preferred instance, the substitution mutation is N297A.

AntiCD20/anti-CD3 bispecific antibody variants are further provided with bisected oligosaccharides, for example, in which a biantennary oligosaccharide attached to the Fc region of the antibody is bisected by GlcNAc. Such antibody variants may have reduced fucosylation and/or improved

ADCC function. Examples of such antibody variants are described, e.g., in WO 2003/011878; U.S. Pat. No. 6,602,684; and U.S. 2005/0123546. Antibody variants with at least one galactose residue in the oligosaccharide attached to the Fc region are also provided. Such antibody variants may have improved CDC function. Such antibody variants are described, e.g., in WO 1997/30087, WO 1998/58964, and WO 1999/22764.

Antibody Derivatives

In certain instances, an anti-CD20/anti-CD3 bispecific antibody of the treatment methods provided herein is further modified to contain additional nonproteinaceous moieties that are known in the art and readily available. The moieties suitable for derivatization of the antibody include, but are not limited to, water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another instance, conjugates of an antibody and nonproteinaceous moiety that may be selectively heated by exposure to radiation are provided. In one instance, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

6. Immunoconjugates

The invention also provides immunoconjugates or antibody drug conjugates comprising an anti-CD20/anti-CD3 bispecific antibody of the invention conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In some instances, an immunoconjugate is an antibody-drug conjugate (ADC) in which an antibody is conjugated to one or more drugs, including but not limited to an auristatin such as monomethylauristatin drug moieties DE and DF (MMAE (vedotin) and MMAF) (see U.S. Pat. Nos. 5,635,483, 5,780,588, 7,498,298, and 8,088,378); a maytansinoid (see U.S. Pat. Nos. 5,208,020, 5,416,064, and European Patent EP 0 425 235 B1); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another instance, an immunoconjugate comprises an anti-CD20/anti-CD3 bispecific antibody conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In another instance, an immunoconjugate comprises an anti-CD20/anti-CD3 bispecific antibody conjugated to a radioactive atom to form a radioconjugate. A variety of radioactive isotopes are available for the production of radioconjugates. Examples include ²¹¹At, ¹³¹I, ¹²⁵I, ⁹⁰Y, ¹⁸⁶Re, ¹⁸⁸Re, ¹⁵³Re, ²¹²Bi, ³²P, ²¹²Pb and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker, or disulfide-containing linker (Chari et al., Cancer Res. 52:127-131 (1992); U.S. Pat. No. 5,208,020) may be used.

The immunoconjugates or ADCs herein expressly contemplate, but are not limited to, such conjugates prepared with cross-linker reagents including, but not limited to, BMPS, EMCS, GMBS, HBVS, LC-SMCC, MBS, MPBH, SBAP, SIA, STAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).

Alternatively, any of the antibodies described herein (e.g., an anti-CD20/anti-CD3 bispecific antibody) can be a naked antibody.

### C. Recombinant Production Methods

The anti-CD20/anti-CD3 bispecific antibodies useful in the combination treatment of the invention may be produced using recombinant methods and compositions, for example, as described in U.S. Pat. No. 4,816,567, which is incorporated herein by reference in its entirety.

For recombinant production of an anti-CD20/anti-CD3 bispecific antibody, nucleic acid encoding an antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N J, 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003).

### (v) CRS Risk Mitigation Strategies

The present invention relates to a new combination treatment of an anti-CD20/anti-CD3 bispecific antibody with an anti-CD20 antibody and one or more chemotherapeutic agents selected from ifosfamide, carboplatin and/or etoposide.

Bispecific antibody therapeutics involving T-cell activation have been associated with cytokine release syndrome (CRS). CRS is a potentially life-threatening symptom complex caused by the excessive release of cytokines by immune effector or target cells during an exaggerated and sustained immune response. CRS can be triggered by a variety of factors, including infection with virulent pathogens, or by medications that activate or enhance the immune response, resulting in a pronounced and sustained immune response.

Regardless of the inciting agent, severe or life-threatening CRS is a medical emergency. If unsuccessfully managed, it can result in significant disability or fatal outcome. Current clinical management focuses on treating the individual signs and symptoms, providing supportive care, and attempting to dampen down the inflammatory response using high-dose corticosteroids. However, this approach is not always successful, especially in the case of late intervention. Moreover, steroids may negatively impact T-cell function, which may diminish the clinical benefit of immune modulating therapies in the treatment of cancer.

### A. CRS Symptoms and Grading

CRS is graded according to the Modified Cytokine Release Syndrome Grading System established by Lee et al., Blood, 124: 188-195, 2014 or Lee et al., Biol Blood Marrow Transplant, 25(4): 625-638, 2019, as described in Table 4. In addition to diagnostic criteria, recommendations on management of CRS based on its severity, including early intervention with corticosteroids and/or anti-cytokine therapy, are provided and referenced in Tables 4 and 5.

TABLE 4
Cytokine release syndrome grading systems

| | Modified Cytokine Release | ASTCT Consensus Grading |
|---|---|---|
| Grade | Syndrome Grading System | System |
| Grade 1 | Symptoms are not life threatening | Temperature ≥ 38° C. |
| | and require symptomatic treatment | No hypotension |
| | only (e.g., fever, nausea, fatigue, | No hypoxia |
| | headache, myalgia, malaise) | |
| Grade 2 | Symptoms require and respond to | Temperature ≥ 38° C.* with |
| | moderate intervention | hypotension not requiring |
| | Oxygen requirement < 40%; or | vasopressors and/or^{†} hypoxia |
| | Hypotension responsive to fluids or | requiring low-flow nasal |
| | low doseª of one vasopressor; or | cannula^{‡} or blow-by |
| | Grade 2 organ toxicity | |
| Grade 3 | Symptoms require and respond to | Temperature ≥ 38° C.* with |
| | aggressive intervention | hypotension requiring a |
| | Oxygen requirement ≥ 40%; or | vasopressor with or without |
| | Hypotension requiring high dose^{b} | vasopressin and/or^{†} hypoxia |
| | or multiple vasopressors; or | requiring high-flow nasal |
| | Grade 3 organ toxicity or Grade 4 | cannula^{‡}, facemask, |
| | transaminitis | nonrebreather mask, or |
| | | Venturi mask |
| Grade 4 | Life-threatening symptoms | Temperature ≥ 38° C.* with |
| | Requirement for ventilation support | hypotension requiring |
| | or | multiple vasopressors |
| | Grade 4 organ toxicity (excluding | (excluding vasopressin) |
| | transaminitis) | and/or^{†} hypoxia |
| | | requiring positive pressure |
| | | (o.g., CPAP, BIPAP, |
| | | intubation and mechanical |
| | | ventilation) |
| Grade 5 | Death | Death |

Lee 2014 criteria: Lee et al., Blood, 124: 188-195, 2014.
ASTCT consensus grading: Lee et al., Biol Blood Marrow Transplant, 25(4): 625-638, 2019.
^{a} Low-dose vasopressor: single vasopressor at doses below that shown in Table 4.
^{b} High-dose vasopressor: as defined in Table 4.
*Fever is defined as temperature ≥38° C. not attributable to any other cause. In patients who have CRS then receive antipyretic or anticytokine therapy such as tocilizumab or steroids, fever is no longer required to grade subsequent CRS severity. In this case, CRS grading is driven by hypotension and/or hypoxia.
^{†}CRS grade is determined by the more severe event: hypotension or hypoxia not attributable to any other cause. For example, a patient with temperature of 39.5° C., hypotension requiring 1 vasopressor, and hypoxia requiring low-flow nasal cannula is classified as grade 3 CRS.
^{‡}Low-flow nasal cannula is defined as oxygen delivered at ≤6 L/minute. Low flow also includes blow-by oxygen delivery, sometimes used in pediatrics. High-flow nasal cannula is defined as oxygen delivered at >6 L/minute.

TABLE 5
High-dose vasopressors
High-Dose Vasopressors (duration ≥ 3 hours)

| Pressor | Dose |
|---|---|
| Norepinephrine monotherapy | ≥20 μg/min |
| Dopamine monotherapy | ≥10 μg/kg/min |
| Phenylephrine monotherapy | ≥200 μg/min |
| Epinephrine monotherapy | ≥10 μg/min |
| If on vasopressin | Vasopressin + norepinephrine |
| | equivalent of ≥10 μg/min ^{a} |
| If on combination or vasopressors | Norepinephrine equivalent |
| (not vasopressin) | of ≥20 μg/min ^{a} |

min = minute;
VASST = Vasopressin and Septic Shock Trial.
^{a} VASST vasopressor equivalent equation: norepinephrine equivalent dose = [norepinephrine (μg/min)] + [dopamine (μg/kg/min) ÷ 2] + [epinephrine (μg/min)] + [phenylephrine (μg/min) ÷ 10].

Mild to moderate presentations of CRS and/or infusion-related reaction (IRR) may include symptoms such as fever, headache, and myalgia, and may be treated symptomatically with analgesics, anti-pyretics, and antihistamines as indicated. Severe or life-threatening presentations of CRS and/or IRR, such as hypotension, tachycardia, dyspnea, or chest discomfort should be treated aggressively with supportive and resuscitative measures as indicated, including the use of high-dose corticosteroids, IV fluids, admission to intensive care unit, and other supportive measures. Severe CRS may be associated with other clinical sequelae such as disseminated intravascular coagulation, capillary leak syndrome, or macrophage activation syndrome (MAS). Standard of care for severe or life-threatening CRS resulting from immune-based therapy has not been established; case reports and recommendations using anti-cytokine therapy such as tocilizumab have been published (Teachey et al., Blood, 121: 5154-5157, 2013; Lee et al., Blood, 124: 188-195, 2014; Maude et al., New Engl J Med, 371: 1507-1517, 2014).

B. Pretreatment or Management of CRS Related Symptoms with Tocilizumab

CRS is associated with high IL-6 levels (Panelli et al., J Transl Med, 2: 17, 2004; Lee et al., Blood, 124: 188-195, 2014; Doessegger and Banholzer, Clin Transl Immunology, 4: e39, 2015), and IL-6 correlates with the severity of CRS, with patients who experience severe or life-threatening CRS (NCI CTCAE Grades 4 or 5) having much higher IL-6 levels compared with their counterparts who do not experience CRS or experience milder CRS reactions (NCI CTCAE Grades 0-3) (Chen et al., J Immunol Methods, 434: 1-8, 2016).

Tocilizumab (ACTEMRA®/ROACTEMRA®) is a recombinant, humanized, anti-human monoclonal antibody directed against soluble and membrane-bound IL-6R, which inhibits IL-6 mediated signaling (see, e.g., WO 1992/019579, which is incorporated herein by reference in its entirety). Tocilizumab has been approved by the U.S. Food and Drug Administration for the treatment of severe or life-threatening CAR-T cell-induced CRS in adults and in pediatric patients 2 years of age and older. Initial clinical data (Locke et al., Blood, 130: 1547, 2017) suggests that tocilizumab prophylaxis may reduce the severity of CAR-T cell-induced CRS by blocking IL-6 receptors from signaling prior to cytokine release. Consequently, tocilizumab premedication may also reduce the frequency or lower the severity of CRS associated with bispecific antibody therapy. Other anti-IL-6R antibodies that could be used in combination with tocilizumab include sarilumab, vobarilizumab (ALX-0061), SA-237, and variants thereof.

In some aspects, an effective amount of tocilizumab is administered as a premedication, e.g., is administered to the subject prior to the administration of the anti-CD20/anti-CD3 bispecific antibody. Administration of tocilizumab as a premedication may reduce the frequency or severity of CRS. In some aspects, tocilizumab is administered as a premedication in Cycle 1, e.g., is administered prior to a first dose (C1D1), a second dose (C1D2), and/or a third dose (C1D3) of the anti-CD20/anti-CD3 bispecific antibody. In some aspects, tocilizumab is administered intravenously to the subject as a single dose of about 1 mg/kg to about 15 mg/kg, e.g., about 4 mg/kg to about 10 mg/kg, e.g., about 6 mg/kg to about 10 mg/kg, e.g., about 8 mg/kg. In some aspects, tocilizumab is administered intravenously to the subject as a single dose of about 8 mg/kg. Other anti-IL-6R antibodies that could be used in combination with tocilizumab include sarilumab, vobarilizumab (ALX-0061), SA-237, and variants thereof.

For example, in one aspect, the anti-CD20/anti-CD3 bispecific antibody is co-administered with tocilizumab (ACTEMRA®/ROACTEMRA®), wherein the subject is first administered with tocilizumab (ACTEMRA®/ROACTEMRA®) and then separately administered with the bispecific antibody (e.g., the subject is pre-treated with tocilizumab (ACTEMRA®/ROACTEMRA®)).

In another aspect, tocilizumab is administered to treat or alleviate symptoms associated with CRS in subjects treated with an anti-CD20/anti-CD3 bispecific antibody. If the subject has a grade 2 or higher CRS event in the presence of extensive comorbidities following administration of the anti-CD20/anti-CD3 bispecific antibody, the method may further include administering to the subject a first dose of an IL-6R antagonist (e.g., an anti-IL-6R antibody, e.g., tocilizumab (ACTEMRA®/ROACTEMRA®)) to manage the grade 2 or higher CRS event while suspending treatment with the anti-CD20/anti-CD3 bispecific antibody. In some instances, the first dose of tocilizumab is administered intravenously to the subject at a dose of about 8 mg/kg. Other anti-IL-6R antibodies that could be used in combination with tocilizumab include sarilumab, vobarilizumab (ALX-0061), SA-237, and variants thereof. In some instances, if the grade 2 or higher CRS event resolves to a grade ≤1 CRS event within two weeks, the method further includes resuming treatment with the anti-CD20/anti-CD3 bispecific antibody at a reduced dose. In some instances, the reduced dose is 50% of the initial infusion rate of the previous cycle if the event occurred during or within 24 hours of the infusion. If, on the other hand, the grade 2 or higher CRS event does not resolve or worsens to a grade ≥3 CRS event within 24 hours of treating the symptoms of the grade 2 or higher CRS event, the method may further include administering to the subject one or more (e.g., one, two, three, four, or five or more) additional doses of an IL-6R antagonist (e.g., an anti-IL-6R antibody, e.g., tocilizumab) to manage the grade 2 or grade ≥3 CRS event. In some particular instances, the grade 2 or higher CRS event does not resolve or worsens to a grade ≥3 CRS event within 24 hours of treating the symptoms of the grade 2 or higher CRS event, and the method may further include administering to the subject one or more additional doses of tocilizumab to manage the grade 2 or grade ≥3 CRS event. In some instances, the one or more additional doses of tocilizumab is administered intravenously to the subject at a dose of about 1 mg/kg to about 15 mg/kg, e.g., about 4 mg/kg to about 10 mg/kg, e.g., about 6 mg/kg to about 10 mg/kg, e.g., about 8 mg/kg.

### C. Other Pretreatments for CRS Risk Mitigation

In one embodiment, the treatment regimen provided herein further comprises administration of premedication prior to the administration of the anti-CD20/anti-CD3 bispecific. In one embodiment the premedication comprises a corticosteroid (such as, e.g., prednisolone, dexamethasone, or methylprednisolone), paracetamol/acetaminophen, and/or an anti-histamine (such as, e.g., diphenhydramine). In one embodiment, the premedication is administered at least 60 minutes (e.g., at least 2, 3, 4, 5, 6, 7, 8, 9, 10, 11, 12, 18, 24, 36, 48 hours, or more) prior to the administration of the anti-CD20/anti-CD3 bispecific antibody. In one embodiment, the treatment regimen further comprises administration of premedication prior to the administration of glofitamab. In embodiment the premedication comprises a corticosteroid (such as, e.g., prednisolone, dexamethasone, or methylprednisolone), an anti-pyretic (such as, e.g., paracetamol/acetaminophen), and/or an anti-histamine (such as, e.g., diphenhydramine). In one embodiment, the subject receives corticosteroid premedication prior to the anti-CD20/anti-CD3 bispecific antibody. It has been shown that premedication using dexamethasone reduced glofitamab-induced cytokine levels in mice pretreated with dexamethasone relative to methylprednisolone. Therefore, in one embodiment, the corticosteroid is dexamethasone. In one embodiment, the premedication is administered at least 60 minutes (e.g., at least 2, 3, 4, 5, 6, 7, 8, 9, 10, 11, 12, 18, 24, 36, 48 hours, or more) prior to the administration of glofitamab. In one embodiment, the premedication is administered at least 60 minutes (e.g., at least 2, 3, 4, 5, 6, 7, 8, 9, 10, 11, 12, 18, 24, 36, 48 hours, or more) prior to each administration of glofitamab. In another embodiment, pre-medication with corticosteroids is administered before the first dose (C1D1) and second dose (C1D2) of the first cycle, before the first dose of the second (C2D1) and third (C3D1) cycle and may be optional for subsequent cycles where the target dose has been reached and tolerated for two doses for patients with no CRS in previous cycles.

In one embodiment, the premedication is administered at least 60 minutes (e.g., at least 2, 3, 4, 5, 6, 7, 8, 9, 10, 11, 12, 18, 24, 36, 48 hours, or more) prior to the administration of the pretreatment with the anti-CD20 antibody, particularly obinutuzumab.

In one embodiment, corticosteroids are administered to manage any relevant adverse events arising after administration of the anti-CD20/anti-CD3 bispecific antibody, e.g., glofitamab.

### (vi) Administration of Anti-CD20/Anti-CD3 Bispecific Antibodies

The methods may involve administering the anti-CD20/anti-CD3 bispecific antibody (and/or any additional therapeutic agent) by any suitable means, including parenteral, intrapulmonary, and intranasal, and, if desired for local treatment, intralesional administration. Parenteral infusions include intravenous, subcutaneous, intramuscular, intraarterial, and intraperitoneal administration routes. In some embodiments, the anti-CD20/anti-CD3 bispecific antibody is administered by intravenous infusion. In one embodiment the infusion time for the anti-CD20/anti-CD3 bispecific antibody, particularly glofitamab, is at least 4 hours (e.g., about 4 hours, about 4.5 hours, about 5 hours, about 5.5 hours, or about 6 hours). In a particular embodiment, the infusion duration for glofitamab is about 4 hours. In one embodiment the infusion time for the anti-CD20/anti-CD3 bispecific antibody may be reduced or extended. In one embodiment (for example, in the absence of infusion-related adverse events), the infusion time of glofitamab in subsequent cycles is reduced to 2 hours ±15 minutes. In one embodiment the infusion time is increased to up to 8 hours (e.g., about 4 hours, about 5 hours, about 6 hours, about 7 hours, or about 8 hours) (for example, for subjects with high risk of experiencing CRS). In one embodiment, for example, for patients who may be at an increased risk of CRS, patients who experience IRRs or CRS with their previous dose of glofitamab or who are at increased risk of recurrent IRR/CRS with subsequent doses, the time of infusion of glofitamab is extended to up to 8 hours.

For all the methods described herein, the anti-CD20/anti-CD3 bispecific antibody would be formulated, dosed, and administered in a fashion consistent with good medical practice. Factors for consideration in this context include the particular disorder being treated, the particular mammal being treated, the clinical condition of the individual subject, the cause of the disorder, the site of delivery of the agent, the method of administration, the scheduling of administration, and other factors known to medical practitioners. The anti-CD20/anti-CD3 bispecific antibody needs not be, but is optionally formulated with, one or more agents currently used to prevent or treat the disorder in question. The effective amount of such other agents depends on the amount of the anti-CD20/anti-CD3 bispecific antibody present in the formulation, the type of disorder or treatment, and other factors discussed above. The anti-CD20/anti-CD3 bispecific antibody may be suitably administered to the subject over a series of treatments.

A further aspect of the present invention relates to the invention as described hereinbefore.

### Embodiments

Some embodiments of the technology described herein can be defined according to any of the following numbered embodiments:

1. A method of treating a subject having a CD20-positive cell proliferative disorder comprising administering to the subject an effective amount of:
(a) a bispecific antibody that binds to CD20 and CD3;
(b) an anti-CD20 antibody; and
(c) one or more chemotherapeutic agents selected from ifosfamide, carboplatin, and/or etoposide in a dosing regimen comprising at least a first dosing cycle and a second dosing cycle.

2. The method of embodiment 1, wherein the subject is aged 18 years or older.

3. The method of embodiment 2, wherein the subject is aged 31 years or older.

4. The method of any one of embodiments 1 to 3, wherein
the first dosing cycle comprises a first dose (C1D1) of the bispecific antibody and a second dose (C1D2) of the bispecific antibody, wherein the C1D1 of the bispecific antibody is about 2.5 mg, and the C1D2 of the bispecific antibody is about 10 mg; and
the second dosing cycle comprises a single dose (C2D1) of the bispecific antibody, wherein the C2D1 of the bispecific antibody is about 10 mg, about 16 mg, or about 30 mg.

5. The method of embodiment 4, wherein the C2D1 of the bispecific antibody is about 30 mg.

6. The method of embodiment 4 or 5, wherein the C1D1 of the bispecific antibody and the C1D2 of the bispecific antibody are administered to the subject on Days 8 and 15, respectively, of the first dosing cycle.

7. The method of any one of embodiments 4 to 6, wherein the C2D1 of the bispecific antibody is administered to the subject on Day 8 of the second dosing cycle.

8. The method of any one of embodiments 1 to 7, wherein the anti-CD20 antibody is obinutuzumab and/or rituximab.

9. The method of embodiment 8, wherein the first dosing cycle comprises a single dose (C1D1) of obinutuzumab; and the second dosing cycle comprises a single dose (C2D1) of rituximab.

10. The method of embodiment 9, wherein the C1D1 of obinutuzumab is about 1000 mg and the C2D1 of rituximab is about 375 mg/m².

11. The method of embodiment 9 or 10, wherein the anti-CD20 antibody is administered in a dosing regimen comprising at least a first dosing cycle and a second dosing cycle, wherein the first dosing cycle comprises administering to the subject the C1D1 of obinutuzumab on Day 1; and the second dosing cycle comprises administering to the subject the C2D1 of rituximab on Day 1.

12. The method of any one of embodiments 1 to 11, wherein the method comprises administering to the subject ifosfamide, carboplatin, and etoposide.

13. The method of embodiment 12, wherein the first dosing cycle comprises a single dose (C1D1) of ifosfamide; a single dose (C1D1) of carboplatin; and a first dose (C1D1) of etoposide, a second dose (C1D2) of etoposide, and a third dose (C1D3) of etoposide; and the second cycle comprises a single dose (C2D1) of ifosfamide; a single dose (C2D1) of carboplatin; and a first dose (C2D1) of etoposide, a second dose (C2D2) of etoposide, and a third dose (C2D3) of etoposide.

14. The method of embodiment 13, wherein ifosfamide is administered at a dose of about 5000 mg/m², about 4000 mg/m², or about 1666 mg/m², carboplatin is administered at a dose in mg to target area under the curve (AUC) of about 5 mg/mL/min with maximum dose of about 750 mg, and etoposide is administered at a dose of about 100 mg/m² or about 75 mg/m² for each dose of etoposide.

15. The method of embodiment 14, wherein ifosfamide is administered at a dose of about 5000 mg/m², carboplatin is administered at a dose in mg to target area under the curve (AUC) of about 5 mg/mL/min with maximum dose of about 750 mg, and etoposide is administered at a dose of about 100 mg/m² for each dose of etoposide.

16. The method of embodiment 13, wherein carboplatin is administered at a dose of about 5×(25+CreatinineClearance(CrCl)) mg with maximum dose of about 750 mg.

17. The method of embodiment 16, wherein
(a) the subject is male, and wherein CrCl is calculated using the formula CrCl=([140−age]×[weight in kg])/(72×[serum creatinine in mg/dL]); or
(b) the subject is female, and wherein CrCl is calculated using the formula CrCl=0.85×([140−age]×[weight in kg])/(72×[serum creatinine in mg/dL]).

18. The method of any one of embodiments 13 to 17, wherein ifosfamide and carboplatin are administered on Day 2 of the first and second dosing cycles and the C1D1-C1D3 and C2D1-C2D3 of etoposide are administered on Days 1, 2, and 3, respectively, of the first and second dosing cycles.

19. The method of any one of embodiments 1 to 18, wherein the first and second dosing cycles are each 21-day dosing cycles.

20. The method of any one of embodiments 1 to 19, wherein the dosing regimen comprises one or more additional dosing cycles.

21. The method of embodiment 20, wherein the one or more additional dosing cycles are each 21-day dosing cycles.

22. The method of embodiment 20 or 21, wherein the dosing regimen comprises three dosing cycles in total.

23. The method of any one of embodiments 20 to 22, wherein the one or more additional dosing cycles each comprises:
(a) an additional single dose of the bispecific antibody that binds to CD20 and CD3,
(b) an additional single dose of the anti-CD20 antibody, and
(c) an additional single dose of ifosfamide, an additional single dose of carboplatin and an additional first dose, an additional second dose, and an additional third dose of etoposide.

24. The method of embodiment 23, wherein the additional single dose of the bispecific antibody is about 30 mg.

25. The method of embodiment 23 or 24, wherein the additional single dose of the bispecific antibody is administered to the subject on Day 8 of each of the one or more additional dosing cycles.

26. The method of any one of embodiments 23 to 25, wherein the anti-CD20 antibody is rituximab.

27. The method of embodiment 26, wherein the additional single dose of rituximab is about 375 mg/m².

28. The method of embodiment 26 or 26, wherein the additional single dose of rituximab is administered on Day 1 of each of the one or more additional dosing cycles.

29. The method of any one of embodiments 23 to 27, wherein the additional single dose of ifosfamide is about 5000 mg/m², about 4000 mg/m², or about 1666 mg/m², the additional single dose of carboplatin is in mg to target area under the curve (AUC) of about 5 mg/mL/min with maximum dose of about 750 mg, and the additional first dose, the additional second dose, and the additional third dose of etoposide are each about 100 mg/m² or about 75 mg/m².

30. The method of embodiment 29, wherein ifosfamide is administered at a dose of about 5000 mg/m², carboplatin is administered at a dose in mg to target area under the curve (AUC) of about 5 mg/mL/min with maximum dose of about 750 mg, and etoposide is administered at a dose of about 100 mg/m² for each dose of etoposide.

31. The method of any one of embodiments 23 to 27, wherein the additional single dose of carboplatin is about 5×(25+CreatinineClearance(CrCl)) mg with maximum dose of about 750 mg.

32. The method of embodiment 31, wherein
(a) the subject is male, and wherein CrCl is calculated using the formula CrCl=([140−age]×[weight in kg])/(72×[serum creatinine in mg/dL]); or
(b) the subject is female, and wherein CrCl is calculated using the formula CrCl=0.85×([140−age]×[weight in kg])/(72×[serum creatinine in mg/dL]).

33. The method of any one of embodiments 23 to 32, wherein ifosfamide and carboplatin are administered on Day 2 of each of the one or more additional dosing cycles and the additional first dose, the additional second dose, and the additional third dose of etoposide are administered on Days 1, 2, and 3, respectively, of each of the one or more additional dosing cycles.

34. The method of any one of embodiments 1 to 33, wherein the method further comprises administering to the subject one or more additional therapeutic agents.

35. The method of embodiment 34, wherein the one or more additional therapeutic agent is tocilizumab.

36. The method of embodiment 35, wherein the weight of the subject is greater than or equal to about 30 kg and tocilizumab is administered at a dose of about 8 mg/kg or the weight of the subject is less than 30 kg and tocilizumab is administered at a dose of about 12 mg/kg, and wherein the maximum dose is about 800 mg.

37. The method of embodiment 34, wherein the one or more additional therapeutic agents is a corticosteroid.

38. The method of embodiment 37, wherein the corticosteroid comprises prednisone, prednisolone, methylprednisolone, or dexamethasone.

39. The method of embodiment 38, wherein the corticosteroid is dexamethasone.

40. The method of embodiment 39, wherein dexamethasone is administered intravenously at a dose of about 20 mg at least about one hour prior to the administration of any dose of the bispecific antibody.

41. The method of embodiment 39, wherein dexamethasone is administered intravenously at a dose of about 20 mg at least about one hour prior to the administration of any dose of obinutuzumab.

42. The method of embodiment 38, wherein the corticosteroid is methylprednisolone.

43. The method of embodiment 42, wherein methylprednisolone is administered intravenously at a dose of about 80 mg at least about one hour prior to the administration of any dose of the bispecific antibody.

44. The method of embodiment 42, wherein methylprednisolone is administered intravenously at a dose of about 80 mg at least about one hour prior to the administration of any dose of obinutuzumab.

45. The method of embodiment 38, wherein the corticosteroid is prednisone.

46. The method of embodiment 45, wherein prednisone is administered orally at a dose of about 100 mg at least about one hour prior to the administration of any dose of the bispecific antibody.

47. The method of embodiment 38, wherein the corticosteroid is prednisolone.

48. The method of embodiment 47, wherein prednisolone is administered intravenously at a dose of about 100 mg at least about one hour prior to the administration of any dose of the bispecific antibody.

49. The method of embodiment 34, wherein the one or more additional therapeutic agents is an antihistamine.

50. The method of embodiment 49, wherein the antihistamine is diphenhydramine.

51. The method of embodiment 50, and wherein diphenhydramine is administered orally or intravenously at a dose of about 50 mg at least about 30 minutes prior to the administration of any dose of the bispecific antibody.

52. The method of embodiment 34, wherein the one or more additional therapeutic agents comprises granulocyte-colony stimulating factor (G-CSF).

53. The method of embodiment 52, wherein G-CSF is administered between about one day and about two days after administration of any dose of rituximab, ifosfamide, carboplatin, and/or etoposide.

54. The method of embodiment 34, wherein the one or more additional therapeutic agents is an antipyretic.

55. The method of embodiment 54, wherein the antipyretic is acetaminophen or paracetamol.

56. The method of embodiment 55, wherein acetaminophen or paracetamol is administered orally at a dose of between about 500 mg to about 1000 mg at least about 30 minutes prior to the administration of any dose of the bispecific antibody.

57. The method of embodiment 55, wherein acetaminophen or paracetamol is administered orally at a dose of between about 500 mg to about 1000 mg at least about 30 minutes prior to the administration of any dose of obinutuzumab.

58. The method of embodiment 34, wherein the one or more additional therapeutic agents is mesna.

59. The method of embodiment 58, wherein mesna is administered at a dose of about 5000 mg/m², about 4000 mg/m², or about 1666 mg/m² intravenously.

60. The method of embodiment 59, wherein mesna is administered via continuous infusion over about 24 hours on Day 2 of each dosing cycle.

61. The method of embodiment 59 or 60, wherein mesna is administered simultaneously with any dose of ifosfamide.

62. A method of treating a subject aged between 6 months and 17 years having a CD20-positive cell proliferative disorder comprising administering to the subject an effective amount of:
(a) a bispecific antibody that binds to CD20 and CD3;
(b) an anti-CD20 antibody; and
(c) one or more chemotherapeutic agents selected from ifosfamide, carboplatin, and/or etoposide in a dosing regimen comprising at least a first dosing cycle and a second dosing cycle.

63. The method of embodiment 62, wherein
the first dosing cycle comprises a first dose (C1D1) of the bispecific antibody and a second dose (C1D2) of the bispecific antibody, wherein the C1D1 of the bispecific antibody is about 0.03 mg/kg, about 0.04 mg/kg, or about 2.5 mg, and the C1D2 of the bispecific antibody is about 0.15 mg/kg or about 10 mg; and
the second dosing cycle comprises a single dose (C2D1) of the bispecific antibody, wherein the C2D1 of the bispecific antibody is about 0.4 mg/kg, about 0.5 mg/kg, or about 30 mg.

64. The method of embodiment 63, wherein:
(a) the subject's body weight is greater than or equal to about 7.5 kg and less than about 13 kg, and wherein the C1D1 of the bispecific antibody is about 0.04 mg/kg, the C1D2 of the bispecific antibody is about 0.15 mg/kg, and the C2D1 of the bispecific antibody is about 0.5 mg/kg;
(b) the subject's body weight is greater than or equal to about 13 kg and less than about 45 kg, and wherein the C1D1 of the bispecific antibody is about 0.03 mg/kg, the C1D2 of the bispecific antibody is about 0.15 mg/kg, and the C2D1 of the bispecific antibody is about 0.4 mg/kg; or
(c) the subject's body weight is greater than or equal to about 45 kg, and wherein the C1D1 of the bispecific antibody is about 2.5 mg, the C1D2 of the bispecific antibody is about 10 mg, and the C2D1 of the bispecific antibody is about 30 mg.

65. The method of embodiment 63 or 64, wherein the C1D1 of the bispecific antibody and the C1D2 of the bispecific antibody are administered to the subject on Days 8 and 15, respectively, of the first dosing cycle.

66. The method of any one of embodiments 63 to 65, wherein the C2D1 of the bispecific antibody is administered to the subject on Day 1 of the second dosing cycle.

67. The method of any one of embodiments 62 to 66, wherein the anti-CD20 antibody is obinutuzumab and/or rituximab.

68. The method of embodiment 67, wherein the first dosing cycle comprises a first dose (C1D1) of obinutuzumab and a second dose (C1D2) of obinutuzumab.

69. The method of embodiment 68, wherein:
(a) the subject's body weight is greater than or equal to about 7.5 kg and less than about 13 kg, and wherein the sum of the C1D1 and the C1D2 of obinutuzumab is about 38 mg/kg;
(b) the subject's body weight is greater than or equal to about 13 kg and less than about 20 kg, and wherein the sum of the C1D1 and the C1D2 of obinutuzumab is about 28 mg/kg;
(c) the subject's body weight is greater than or equal to about 20 kg and less than about 32 kg, and wherein the sum of the C1D1 and the C1D2 of obinutuzumab is about 23 mg/kg;
(d) the subject's body weight is greater than or equal to about 32 kg and less than about 45 kg, and wherein the sum of the C1D1 and the C1D2 of obinutuzumab is about 20 mg/kg; or
(e) the subject's body weight is greater than or equal to about 45 kg, and wherein the sum of the C1D1 and the C1D2 of obinutuzumab is about 1000 mg.

70. The method of embodiment 68 or 69, wherein the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab.

71. The method of any one of embodiments 68 to 70, wherein:
(a) the subject's body weight is greater than or equal to about 7.5 kg and less than about 13 kg, and wherein the C1D1 of obinutuzumab is about 3.8 mg/kg and the C1D2 of obinutuzumab is about 34.2 mg/kg;
(b) the subject's body weight is greater than or equal to about 13 kg and less than about 20 kg, and wherein the C1D1 of obinutuzumab is about 2.8 mg/kg and the C1D2 of obinutuzumab is about 35.2 mg/kg;
(c) the subject's body weight is greater than or equal to about 20 kg and less than about 32 kg, and wherein the C1D1 of obinutuzumab is about 2.3 mg/kg and the C1D2 of obinutuzumab is about 35.7 mg/kg;
(d) the subject's body weight is greater than or equal to about 32 kg and less than about 45 kg, and wherein the C1D1 of obinutuzumab is about 2.0 mg/kg and the C1D2 of obinutuzumab is about 36.0 mg/kg; or
(e) the subject's body weight is greater than or equal to about 45 kg, and wherein the C1D1 of obinutuzumab is about 100 mg and the C1D2 of obinutuzumab is about 900 mg.

72. The method of any one of embodiments 68 to 71, wherein the C1D1 of obinutuzumab is administered to the subject on Day 1 of the first dosing cycle and the C1D2 of obinutuzumab is administered to the subject on Day 2 of the first dosing cycle.

73. The method of embodiment 67, wherein the second dosing cycle comprises a single dose (C2D1) of rituximab.

74. The method of embodiment 73, wherein the C2D1 of rituximab is about 375 mg/m².

75. The method of embodiment 73 or 74, wherein rituximab is administered to the subject on Day 5 of the second dosing cycle.

76. The method of any one of embodiments 62 to 75, wherein the method comprises administering to the subject ifosfamide, carboplatin, and etoposide.

77. The method of embodiment 76, wherein the first dosing cycle comprises:
(a) a first dose (C1D1) of ifosfamide, a second dose (C1D2) of ifosfamide, and a third dose (C1D3) of ifosfamide;
(b) a single dose (C1D1) of carboplatin; and
(c) a first dose (C1D1) of etoposide, a second dose (C1D2) of etoposide, and a third dose (C1D3) of etoposide;
and the second cycle comprises:
(a) a first dose (C2D1) of ifosfamide, a second dose (C2D2) of ifosfamide, and a third dose (C2D3) of ifosfamide;
(b) a single dose (C2D1) of carboplatin; and
(c) a first dose (C2D1) of etoposide, a second dose (C2D2) of etoposide, and a third dose (C2D3) of etoposide.

78. The method of embodiment 77, wherein ifosfamide is administered at a dose of about 3000 mg/m² for each dose of ifosfamide, carboplatin is administered at a dose of about 635 mg/m², and etoposide is administered at a dose of about 100 mg/m² for each dose of etoposide.

79. The method of embodiment 77 or 78, wherein:
(a) the C1D1, C1D2, and C1D3 of ifosfamide are administered on Days 3, 4, and 5, respectively of the first dosing cycle;
(b) the C1D1 of carboplatin is administered on Day 3 of the first dosing cycle;
(c) the C1D1, C1D2, and C1D3 of etoposide are administered on Days 3, 4, and 5, respectively, of the first dosing cycle;
(d) the C2D1, C2D2, and C2D3 of ifosfamide are administered on Days 6, 7, and 8, respectively, of the second dosing cycle;
(e) the C2D1 of carboplatin is administered on Day 6 of the second dosing cycle; and
(f) the C2D1, C2D2, and C2D3 of etoposide are administered on Days 6, 7, and 8, respectively, of the second dosing cycle.

80. The method of any one of embodiments 62 to 79, wherein the first and second dosing cycles are each 21-day dosing cycles.

81. The method of any one of embodiments 62 to 80, wherein the dosing regimen comprises one or more additional dosing cycles.

82. The method of embodiment 81, wherein the one or more additional dosing cycles are each 21-day dosing cycles.

83. The method of embodiment 81 or 82, wherein the dosing regimen comprises three dosing cycles in total.

84. The method of any one of embodiments 81 to 83, wherein the one or more additional dosing cycles each comprises:
(a) an additional single dose of the bispecific antibody that binds to CD20 and CD3,
(b) an additional single dose of the anti-CD20 antibody, and
(c) an additional first dose, an additional second dose, and an additional third dose of ifosfamide; an additional single dose of carboplatin; and an additional first dose, an additional second dose, and an additional third dose of etoposide.

85. The method of embodiment 84, wherein:
(a) the subject's body weight is greater than or equal to about 7.5 kg and less than about 13 kg, and wherein the additional single dose of the bispecific antibody is about 0.5 mg/kg;
(b) the subject's body weight is greater than or equal to about 13 kg and less than about 45 kg, and wherein the additional single dose of the bispecific antibody is about 0.4 mg/kg; or
(c) the subject's body weight is greater than or equal to about 45 kg, and wherein the additional single dose of the bispecific antibody is about 30 mg.

86. The method of embodiment 84 or 85, wherein the additional single dose of the bispecific antibody is administered to the subject on Day 1 of each of the one or more additional dosing cycles.

87. The method of any one of embodiments 84 to 86, wherein the anti-CD20 antibody is rituximab.

88. The method of embodiment 87, wherein the additional single dose of rituximab is about 375 mg/m².

89. The method of embodiment 87 or 88, wherein the additional single dose of rituximab is administered on Day 5 of each of the one or more additional dosing cycles.

90. The method of any one of embodiments 81 to 89, wherein the additional first dose, additional second dose, and additional third dose of ifosfamide are each about 3000 mg/m², the additional single dose of carboplatin is about 635 mg/m², and the additional first dose, the additional second dose, and the additional third dose of etoposide are each about 100 mg/m².

91. The method of any one of embodiments 81 to 90, wherein:
(a) the additional first dose, the additional second dose, and the additional third dose of ifosfamide are administered to the subject on Days 6, 7, and 8, respectively, of each of the one or more additional dosing cycles;
(b) the additional single dose of carboplatin is administered on Day 6 of each of the one or more additional dosing cycles; and
(c) the additional first dose, the additional second dose, and the additional third dose of etoposide are administered to the subject on Days 6, 7, and 8, respectively, of each of the one or more additional dosing cycles.

92. The method of any one of embodiments 62 to 91, wherein the method further comprises administering to the subject one or more additional therapeutic agents.

93. The method of embodiment 92, wherein the one or more additional therapeutic agent is tocilizumab.

94. The method of embodiment 93, wherein the weight of the subject is greater than or equal to about 30 kg and tocilizumab is administered at a dose of about 8 mg/kg or the weight of the subject is less than 30 kg and tocilizumab is administered at a dose of about 12 mg/kg, and wherein the maximum dose is about 800 mg.

95. The method of embodiment 92, wherein the one or more additional therapeutic agents is a corticosteroid.

96. The method of embodiment 95, wherein the corticosteroid comprises prednisone, prednisolone, methylprednisolone, or dexamethasone.

97. The method of embodiment 96, wherein the corticosteroid is dexamethasone.

98. The method of embodiment 97, wherein dexamethasone is administered intravenously at a dose of between about 0.15 mg/kg to about 0.5 mg/kg at least about one hour prior to the administration of any dose of the bispecific antibody, and wherein the maximum daily dose is 10 mg.

99. The method of embodiment 97, wherein dexamethasone is administered intravenously at a dose of between about 0.15 mg/kg to about 0.5 mg/kg at least about one hour prior to the administration of any dose of obinutuzumab, and wherein the maximum daily dose is 10 mg.

100. The method of embodiment 96, wherein the corticosteroid is methylprednisolone.

101. The method of embodiment 100, wherein methylprednisolone is administered intravenously at a dose of between about 1 mg/kg to about 2 mg/kg at least about one hour prior to the administration of any dose of the bispecific antibody.

102. The method of embodiment 100, wherein methylprednisolone is administered intravenously at a dose of between about 1 mg/kg to about 2 mg/kg at least about one hour prior to the administration of any dose of obinutuzumab.

103. The method of embodiment 96, wherein the corticosteroid is prednisone or prednisolone.

104. The method of embodiment 103, wherein prednisone or prednisolone is administered intravenously at a dose of about 100 mg or about 2 mg/kg at least about one hour prior to the administration of any dose of the bispecific antibody.

105. The method of embodiment 103, wherein prednisone or prednisolone is administered intravenously at a dose of about 100 mg or about 2 mg/kg at least about one hour prior to the administration of any dose of obinutuzumab.

106. The method of embodiment 92, wherein the one or more additional therapeutic agents is an antihistamine.

107. The method of embodiment 106, wherein the antihistamine is diphenhydramine.

108. The method of embodiment 107, wherein the subject is aged between two years and 17 years, and wherein diphenhydramine is administered intravenously at a dose of between about 10 mg to 20 mg with a maximum single dose of about 1.25 mg/kg.

109. The method of embodiment 107, wherein the subject is aged less than two years, and wherein diphenhydramine is administered rectally at a dose of about 20 mg.

110. The method of embodiment 108 or 109, wherein diphenhydramine is administered at least about 30 minutes prior to the administration of any dose of the bispecific antibody and/or the anti-CD20 antibody.

111. The method of embodiment 92, wherein the one or more additional therapeutic agents comprises granulocyte-colony stimulating factor (G-CSF).

112. The method of embodiment 111, wherein G-CSF is administered between about one day and about two days after administration of any dose of rituximab, ifosfamide, carboplatin, and/or etoposide.

113. The method of embodiment 112, wherein G-CSF is administered intravenously or subcutaneously at a dose of about 5 μg/kg/day or about 10 μg/kg/day.

114. The method of embodiment 113, wherein G-CSF is administered at a dose of about 5 μg/kg/day in the first dosing cycle and about 10 μg/kg/day in the second dosing cycle and/or each additional dosing cycle.

115. The method of embodiment 92, wherein the one or more additional therapeutic agents is an antipyretic.

116. The method of embodiment 115, wherein the antipyretic is acetaminophen or paracetamol.

117. The method of embodiment 116, wherein acetaminophen or paracetamol is administered orally or intravenously at a dose of between about 500 to about 1000 mg.

118. The method of embodiment 117, wherein acetaminophen or paracetamol is administered at least about 30 minutes prior to the administration of any dose of the bispecific antibody and/or the anti-CD20 antibody.

119. The method of embodiment 92, wherein the one or more additional therapeutic agents is mesna.

120. The method of embodiment 119, wherein mesna is administered intravenously daily as five doses totaling 3000 mg/m² in amount.

121. The method of embodiment 120, wherein mesna is administered intravenously at a first dose of about 600 mg/m² prior to the administration of any dose of ifosfamide and at four repeated doses of about 600 mg/m² each at about three hours, about six hours, about nine hours, and about 12 hours, respectively, after the first dose of ifosfamide.

122. The method of embodiment 120 or 121, wherein mesna is administered daily to the subject on Days 3, 4, and 5 of the first dosing cycle, on Days 6, 7, and 8 of the second dosing cycle, and/or on Days 6, 7, and 8 of each additional dosing cycle.

123. A method of treating a subject aged between 18 years and 30 years having a CD20-positive cell proliferative disorder comprising administering to the subject an effective amount of:
(a) a bispecific antibody that binds to CD20 and CD3;
(b) an anti-CD20 antibody; and
(c) one or more chemotherapeutic agents selected from ifosfamide, carboplatin, and/or etoposide in a dosing regimen comprising at least a first dosing cycle and a second dosing cycle.

124. The method of embodiment 123, wherein
the first dosing cycle comprises a first dose (C1D1) of the bispecific antibody and a second dose (C1D2) of the bispecific antibody, wherein the C1D1 of the bispecific antibody is about 2.5 mg, and the C1D2 of the bispecific antibody is about 10 mg; and
the second dosing cycle comprises a single dose (C2D1) of the bispecific antibody, wherein the C2D1 of the bispecific antibody is about 30 mg.

125. The method of embodiment 124, wherein the C1D1 of the bispecific antibody and the C1D2 of the bispecific antibody are administered to the subject on Days 8 and 15, respectively, of the first dosing cycle.

126. The method of embodiment 124 or 125, wherein the C2D1 of the bispecific antibody is administered to the subject on Day 1 of the second dosing cycle.

127. The method of any one of embodiments 123 to 126, wherein the anti-CD20 antibody is obinutuzumab and/or rituximab.

128. The method of embodiment 127, wherein the first dosing cycle comprises a first dose (C1D1) of obinutuzumab and a second dose (C1D2) of obinutuzumab.

129. The method of embodiment 128, wherein the sum of the C1D1 and the C1D2 of obinutuzumab is about 1000 mg.

130. The method of embodiment 128 or 129, wherein the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab.

131. The method of any one of embodiments 128 to 130, wherein the C1D1 of obinutuzumab is about 100 mg and the C1D2 of obinutuzumab is about 900 mg.

132. The method of any one of embodiments 128 to 131, wherein the C1D1 of obinutuzumab is administered to the subject on Day 1 of the first dosing cycle and the C1D2 of obinutuzumab is administered to the subject on Day 2 of the first dosing cycle.

133. The method of embodiment 127, wherein the second dosing cycle comprises a single dose (C2D1) of rituximab.

134. The method of embodiment 133, wherein the C2D1 of rituximab is about 375 mg/m².

135. The method of embodiment 133 or 134, wherein the C2D1 of rituximab is administered to the subject on Day 5 of the second dosing cycle.

136. The method of any one of embodiments 133 to 135, wherein the method comprises administering to the subject ifosfamide, carboplatin, and etoposide.

137. The method of embodiment 136, wherein the first dosing cycle comprises:
(a) a single dose (C1D1) of ifosfamide;
(b) a single dose (C1D1) of carboplatin; and
(c) a first dose (C1D1) of etoposide, a second dose (C1D2) of etoposide, and a third dose (C1D3) of etoposide;
and the second cycle comprises:
(a) a single dose (C2D1) of ifosfamide;
(b) a single dose (C2D1) of carboplatin; and
(c) a first dose (C2D1) of etoposide, a second dose (C2D2) of etoposide, and a third dose (C2D3) of etoposide.

138. The method of embodiment 137, wherein ifosfamide is administered at a dose of about 5000 mg/m², carboplatin is administered at a dose of about 5×(25+CreatinineClearance (CrCl)) mg with maximum dose of about 750 mg, and etoposide is administered at a dose of about 100 mg/m² for each dose of etoposide.

139. The method of embodiment 138, wherein:
(a) the subject is male, and wherein CrCl is calculated using the formula CrCl=([140−age]×[weight in kg])/(72×[serum creatinine in mg/dL]); or
(b) the subject is female, and wherein CrCl is calculated using the formula CrCl=0.85×([140−age]×[weight in kg])/(72×[serum creatinine in mg/dL]).

140. The method of embodiment 138 or 139, wherein:
(a) the subject has CrCl<about 60 mL/min, and wherein each single dose of ifosfamide is reduced to 4000 mg/m²; and/or
(b) the subject has CrCl<about 50 mL/min, and wherein each dose of etoposide is reduced to about 75 mg/m².

141. The method of embodiment any one of embodiments 137 to 140, wherein:
(a) the C1D1 ifosfamide is administered on Day 3 of the first dosing cycle;
(b) the C1D1 of carboplatin is administered on Day 3 of the first dosing cycle;
(c) the C1D1, C1D2, and C1D3 of etoposide are administered on Days 3, 4, and 5, respectively, of the first dosing cycle;
(d) the C2D1 of ifosfamide is administered on Day 6 of the second dosing cycle;
(e) the C2D1 of carboplatin is administered on Day 6 of the second dosing cycle; and
(f) the C2D1, C2D2, and C2D3 of etoposide are administered on Days 6, 7, and 8, respectively, of the second dosing cycle.

142. The method of any one of embodiments 123 to 141, wherein the first and second dosing cycles are each 21-day dosing cycles.

143. The method of any one of embodiments 123 to 142, wherein the dosing regimen comprises one or more additional dosing cycles.

144. The method of embodiment 143, wherein the one or more additional dosing cycles are each 21-day dosing cycles.

145. The method of embodiment 143 or 144, wherein the dosing regimen comprises three dosing cycles in total.

146. The method of any one of embodiments 143 to 145, wherein the one or more additional dosing cycles each comprise:
(a) an additional single dose of the bispecific antibody that binds to CD20 and CD3,
(b) an additional single dose of the anti-CD20 antibody, and
(c) an additional single dose of ifosfamide; an additional single dose of carboplatin; and an additional first dose, an additional second dose, and an additional third dose of etoposide.

147. The method of embodiment 146, wherein the additional single dose of the bispecific antibody is about 30 mg.

148. The method of embodiment 146 or 147, wherein the additional single dose of the bispecific antibody is administered to the subject on Day 1 of each of the one or more additional dosing cycles.

149. The method of any one of embodiments 146 to 148, wherein the anti-CD20 antibody is rituximab.

150. The method of embodiment 149, wherein the additional single dose of rituximab is about 375 mg/m².

151. The method of embodiment 149 or 150, wherein the additional single dose of rituximab is administered on Day 5 of each of the one or more additional dosing cycles.

152. The method of any one of embodiments 146 to 151, wherein the additional single dose of ifosfamide is about 5000 mg/m², the additional single dose of carboplatin is about 5×(25+CreatinineClearance (CrCl)) mg with maximum dose of about 750 mg, and the additional first dose, the additional second dose, and the additional third dose of etoposide are each about 100 mg/m².

153. The method of embodiment 152, wherein:
(a) the subject is male, and wherein CrCl is calculated using the formula CrCl=([140−age]×[weight in kg])/(72×[serum creatinine in mg/dL]); or
(b) the subject is female, and wherein CrCl is calculated using the formula CrCl=0.85×([140−age]×[weight in kg])/(72×[serum creatinine in mg/dL]).

154. The method of embodiment 152 or 153, wherein:
(a) the subject has CrCl<about 60 mL/min, and wherein the additional single dose of ifosfamide is reduced to 4000 mg/m²; and/or
(b) the subject has CrCl<about 50 mL/min, and wherein each additional dose of etoposide is reduced to about 75 mg/m².

155. The method of any one of embodiments 146 to 154, wherein:
(a) the additional single dose of ifosfamide is administered on Day 6 of each of the one or more additional dosing cycles;
(b) the additional single dose of carboplatin is administered on Day 6 of each of the one or more additional dosing cycles; and
(c) the additional first dose, the additional second dose, and the additional third dose of etoposide are administered to the subject on Days 6, 7, and 8, respectively, of each of the one or more additional dosing cycles.

156. The method of any one of embodiments 123 to 155, wherein the method further comprises administering to the subject one or more additional therapeutic agents.

157. The method of embodiment 156, wherein the one or more additional therapeutic agent is tocilizumab.

158. The method of embodiment 157, wherein the weight of the subject is greater than or equal to about 30 kg and tocilizumab is administered at a dose of about 8 mg/kg or the weight of the subject is less than 30 kg and tocilizumab is administered at a dose of about 12 mg/kg, and wherein the maximum dose is about 800 mg.

159. The method of embodiment 156, wherein the one or more additional therapeutic agents is a corticosteroid.

160. The method of embodiment 159, wherein the corticosteroid comprises prednisone, prednisolone, methylprednisolone, or dexamethasone.

161. The method of embodiment 160, wherein the corticosteroid is dexamethasone.

162. The method of embodiment 161, wherein dexamethasone is administered intravenously at a dose of between about 0.15 mg/kg to about 0.5 mg/kg at least about one hour prior to the administration of any dose of the bispecific antibody, and wherein the maximum daily dose is 10 mg.

163. The method of embodiment 161, wherein dexamethasone is administered intravenously at a dose of between about 0.15 mg/kg to about 0.5 mg/kg at least about one hour prior to the administration of any dose of obinutuzumab, and wherein the maximum daily dose is 10 mg.

164. The method of embodiment 160, wherein the corticosteroid is methylprednisolone.

165. The method of embodiment 164, wherein methylprednisolone is administered intravenously at a dose of between about 1 mg/kg to about 2 mg/kg at least about one hour prior to the administration of any dose of the bispecific antibody.

166. The method of embodiment 164, wherein methylprednisolone is administered intravenously at a dose of between about 1 mg/kg to about 2 mg/kg at least about one hour prior to the administration of any dose of obinutuzumab.

167. The method of embodiment 160, wherein the corticosteroid is prednisone or prednisolone.

168. The method of embodiment 167, wherein prednisone or prednisolone is administered intravenously at a dose of about 100 mg or about 2 mg/kg at least about one hour prior to the administration of any dose of the bispecific antibody.

169. The method of embodiment 167, wherein prednisone or prednisolone is administered intravenously at a dose of about 100 mg or about 2 mg/kg at least about one hour prior to the administration of any dose of obinutuzumab.

170. The method of embodiment 156, wherein the one or more additional therapeutic agents is an antihistamine.

171. The method of embodiment 170, wherein the antihistamine is diphenhydramine.

172. The method of embodiment 171, wherein diphenhydramine is administered orally or intravenously at a dose of about 50 mg.

173. The method of embodiment 172, wherein diphenhydramine is administered at least about 30 minutes prior to the administration of any dose of the bispecific antibody and/or the anti-CD20 antibody.

174. The method of embodiment 156, wherein the one or more additional therapeutic agents comprises granulocyte-colony stimulating factor (G-CSF).

175. The method of embodiment 174, wherein G-CSF is administered between about one day and about two days after administration of any dose of rituximab, ifosfamide, carboplatin, and/or etoposide.

176. The method of embodiment 175, wherein G-CSF is administered intravenously or subcutaneously at a dose of about 5 μg/kg/day or about 10 μg/kg/day.

177. The method of embodiment 176, wherein G-CSF is administered at a dose of about 5 μg/kg/day in the first dosing cycle and about 10 μg/kg/day in the second dosing cycle and/or each additional dosing cycle.

178. The method of embodiment 152, wherein the one or more additional therapeutic agents is an antipyretic.

179. The method of embodiment 178, wherein the antipyretic is acetaminophen or paracetamol.

180. The method of embodiment 179, wherein acetaminophen or paracetamol is administered orally or intravenously at a dose of between about 500 to about 1000 mg.

181. The method of embodiment 180, wherein acetaminophen or paracetamol is administered at least about 30 minutes prior to the administration of any dose of the bispecific antibody and/or the anti-CD20 antibody.

182. The method of embodiment 156, wherein the one or more additional therapeutic agents is mesna.

183. The method of embodiment 182, wherein mesna is administered intravenously at a dose of about 5000 mg/m².

184. The method of embodiment 183, wherein mesna is administered via continuous infusion over about 24 hours on Day 3 of the first dosing cycle, on Day 6 of the second dosing cycle, and/or on Day 6 of each additional dosing cycle.

185. The method of embodiment 183 or 184, wherein mesna is administered simultaneously with any dose of ifosfamide.

186. The method of any one of embodiments 1 to 185, wherein the bispecific antibody comprises at least one Fab molecule which specifically binds to CD20 comprising the following six hypervariable regions (HVRs):
(i) an HVR-H1 comprising the amino acid sequence of YSWIN (SEQ ID NO: 1);
(ii) an HVR-H2 comprising the amino acid sequence of RIFPGDGDTDYNGKFKG (SEQ ID NO: 2);
(iii) an HVR-H3 comprising the amino acid sequence of NVFDGYWLVY (SEQ ID NO:3);
(iv) an HVR-L1 comprising the amino acid sequence of RSSKSLLHSNGITYLY (SEQ ID NO: 4);
(v) an HVR-L2 comprising the amino acid sequence of QMSNLVS (SEQ ID NO: 5); and
(vi) an HVR-L3 comprising the amino acid sequence of AQNLELPYT (SEQ ID NO: 6).

187. The method of any one of embodiments 1 to 186, wherein the bispecific antibody comprises at least one Fab molecule which specifically binds to CD20 comprising (a) a heavy chain variable (VH) domain comprising an amino acid sequence having at least 95% sequence identity to the amino acid sequence of SEQ ID NO: 7; (b) a light chain variable (VL) domain comprising an amino acid sequence having at least 95% sequence identity to the amino acid sequence of SEQ ID NO: 8; or (c) a VH domain as in (a) and a VL domain as in (b).

188. The method of embodiment 187, wherein the Fab molecule which specifically binds to CD20 comprises (a) a VH domain comprising the amino acid sequence of SEQ ID NO: 7 and (b) a VL domain comprising the amino acid sequence of SEQ ID NO: 8.

189. The method of any one of embodiments 1 to 188, wherein the bispecific antibody comprises at least one Fab molecule which specifically binds to CD3 comprising the following six HVRs:
(i) an HVR-H1 comprising the amino acid sequence of TYAMN (SEQ ID NO: 9);
(ii) an HVR-H2 comprising the amino acid sequence of RIRSKYNNYATYYADSVKG (SEQ ID NO: 10);
(iii) an HVR-H3 comprising the amino acid sequence of HGNFGNSYVSWFAY (SEQ ID NO: 11);
(iv) an HVR-L1 comprising the amino acid sequence of GSSTGAVTTSNYAN (SEQ ID NO: 12);
(v) an HVR-L2 comprising the amino acid sequence of GTNKRAP (SEQ ID NO: 13); and
(vi) an HVR-L3 comprising the amino acid sequence of ALWYSNLWV (SEQ ID NO: 14).

190. The method of any one of embodiments 1 to 189, wherein the bispecific antibody comprises at least one Fab molecule which specifically binds to CD3 comprising (a) a VH domain comprising an amino acid sequence having at least 95% sequence identity to the amino acid sequence of SEQ ID NO: 15; (b) a VL domain comprising an amino acid sequence having at least 95% sequence identity to the amino acid sequence of SEQ ID NO: 16; or (c) a VH domain as in (a) and a VL domain as in (b).

191. The method of embodiment 190, wherein the Fab molecule which specifically binds to CD3 comprises (a) a VH domain comprising the amino acid sequence of SEQ ID NO: 15 and (b) a VL domain comprising the amino acid sequence of SEQ ID NO: 16.

192. The method of any one of embodiments 1 to 191, wherein the bispecific antibody is bivalent for CD20 and monovalent for CD3.

193. The method of any one of embodiments 1 to 192, wherein the bispecific antibody comprises two Fab molecules which specifically bind to CD20 and one Fab molecule which specifically binds to CD3.

194. The method of any one of embodiments 1 to 193, wherein the bispecific antibody is a humanized antibody.

195. The method of any one of embodiments 1 to 194, wherein the bispecific antibody is glofitamab. 196. The method of any one of embodiments 1 to 195, wherein the bispecific antibody is administered intravenously.

197. The method of any one of embodiments 1 to 196, wherein the anti-CD20 antibody is administered intravenously.

198. The method of any one of embodiments 1 to 197, wherein ifosfamide, carboplatin, and/or etoposide are administered intravenously.

199. The method of any one of embodiments 1 to 198, wherein the CD20-positive cell proliferative disorder is a B cell proliferative disorder.

200. The method of embodiment 199, wherein the B cell proliferative disorder is a non-Hodgkin's lymphoma (NHL) or a central nervous system lymphoma (CNSL).

201. The method of embodiment 200, wherein the NHL is a diffuse-large B cell lymphoma (DLBCL), a follicular lymphoma (FL), a mantle cell lymphoma (MCL), a marginal zone lymphoma (MZL), a high-grade B cell lymphoma, a primary mediastinal (thymic) large B cell lymphoma (PMLBCL), a diffuse B cell lymphoma, or a small lymphocytic lymphoma.

202. The method of embodiment 200, wherein the NHL is a Burkitt lymphoma (BL) or a Burkitt leukemia (BAL).

203. The method of any one of embodiments 200 to 202, wherein the NHL is relapsed and/or refractory.

204. The method of any one of embodiments 200 to 203, wherein the NHL is aggressive and/or mature.

205. The method of embodiment 200, wherein the B cell proliferative disorder is a relapsed and/or refractory mature B cell NHL.

206. The method of embodiment 203, wherein the subject has received one prior systemic therapy.

207. The method of embodiment 206, wherein the subject has received no more than one prior systemic therapy.

208. The method of embodiment 206 or 207, wherein the prior systemic therapy comprises an anti-CD20 antibody and an anthracycline.

209. The method of any one of embodiments 1 to 208, wherein the subject is human.

210. The method of any one of embodiments 1 to 209, wherein the subject is transplant or CAR-T cell therapy eligible.

211. The method of embodiment 210, wherein the subject receives autologous stem cell transplantation (ASCT) after completion of the dosing regimen of any one of embodiments 1 to 209.

212. The method of embodiment 211, wherein the ASCT is an autologous hematopoietic stem cell transplant.

213. The method of embodiment 210, wherein the subject receives allogenic hematopoietic stem cell transplantation after completion of the dosing regimen of any one of embodiments 1 to 209.

214. The method of embodiment 210, wherein the subject receives CAR-T cell therapy after completion of the dosing regimen of any one of embodiments 1 to 209.

215. A method of treating a subject having a CD20-positive cell proliferative disorder comprising administering to the subject an effective amount of glofitamab, obinutuzumab, rituximab, ifosfamide, carboplatin, and etoposide in a dosing regimen comprising at least a first dosing cycle and a second dosing cycle, wherein:
(a) the first dosing cycle comprises administering a first dose (C1D1) of glofitamab on Day 8 and administering a second dose (C1D2) of glofitamab on Day 15, wherein the C1D1 of glofitamab is about 2.5 mg, and the C1D2 of glofitamab is about 10 mg; and
(b) the second dosing cycle comprises administering a single dose (C2D1) of glofitamab on Day 8, wherein the C2D1 of glofitamab is about 30 mg.

216. A method of treating a subject having a CD20-positive cell proliferative disorder comprising administering to the subject an effective amount of glofitamab, obinutuzumab, rituximab, ifosfamide, carboplatin, and etoposide in a dosing regimen comprising a first dosing cycle, a second dosing cycle, and a third dosing cycle, wherein:
(a) the first dosing cycle comprises administering a first dose (C1D1) of glofitamab on Day 8 and administering a second dose (C1D2) of glofitamab on Day 15, wherein the C1D1 of glofitamab is about 2.5 mg, and the C1D2 of glofitamab is about 10 mg;
(b) the second dosing cycle comprises administering a single dose (C2D1) of glofitamab on Day 8, wherein the C2D1 of glofitamab is about 30 mg; and
(c) the third dosing cycle comprises administering a single dose (C3D1) of glofitamab on Day 8, wherein the C3D1 of glofitamab is about 30 mg.

217. A method of treating a subject having a CD20-positive cell proliferative disorder comprising administering to the subject an effective amount of glofitamab, obinutuzumab, rituximab, ifosfamide, carboplatin, and etoposide in a dosing regimen comprising at least a first dosing cycle and a second dosing cycle, wherein:
(a) the first dosing cycle comprises:
   (i) administering a first dose (C1D1) of glofitamab on Day 8 and a second dose (C1D2) of glofitamab on Day 15, wherein the C1D1 of glofitamab is about 2.5 mg, and the C1D2 of glofitamab is about 10 mg;
   (ii) administering a first dose (C1D1) of obinutuzumab on Day 1, wherein the C1D1 of obinutuzumab is about 1000 mg;
   (iii) administering a single dose (C1D1) of ifosfamide on Day 2, wherein the C1D1 of ifosfamide is about 5000 mg/m² with a maximum dose of about 800 mg;
   (iv) administering a single dose (C1D1) of carboplatin on Day 2, wherein the C1D1 of carboplatin is about 5×(25+creatinine clearance) mg; and
   (v) administering a first dose (C1D1) of etoposide on Day 1, a second dose (C1D2) of etoposide on Day 2, and a third dose (C1D3) of etoposide on Day 3, wherein the C1D1, the C1D2, and the C1D3 of etoposide are each about 100 mg/m²; and
(b) the second dosing cycle comprises:
   (i) administering a single dose (C2D1) of glofitamab on Day 8, wherein the C2D1 of glofitamab is about 30 mg;
   (ii) administering a first dose (C2D1) of rituximab on Day 1, wherein the C2D1 of rituximab is about 375 mg/m²;
   (iii) administering a single dose (C2D1) of ifosfamide on Day 2, wherein the C2D1 of ifosfamide is about 5000 mg/m² with a maximum dose of about 800 mg;
   (iv) administering a single dose (C2D1) of carboplatin on Day 2, wherein the C2D1 of carboplatin is about 5×(25+creatinine clearance) mg; and
   (v) administering a first dose (C2D1) of etoposide on Day 1, a second dose (C2D2) of etoposide on Day 2, and a third dose (C2D3) of etoposide on Day 3, wherein the C2D1, the C2D2, and the C2D3 of etoposide are each about 100 mg/m².

218. A method of treating a subject having a CD20-positive cell proliferative disorder comprising administering to the subject an effective amount of glofitamab, obinutuzumab, rituximab, ifosfamide, carboplatin, and etoposide in a dosing regimen comprising a first dosing cycle, a second dosing cycle, and a third dosing cycle, wherein:
(a) the first dosing cycle comprises:
   (i) administering a first dose (C1D1) of glofitamab on Day 8 and a second dose (C1D2) of glofitamab on Day 15, wherein the C1D1 of glofitamab is about 2.5 mg, and the C1D2 of glofitamab is about 10 mg;
   (ii) administering a first dose (C1D1) of obinutuzumab on Day 1, wherein the C1D1 of obinutuzumab is about 1000 mg;
   (iii) administering a single dose (C1D1) of ifosfamide on Day 2, wherein the C1D1 of ifosfamide is about 5000 mg/m² with a maximum dose of about 800 mg;
   (iv) administering a single dose (C1D1) of carboplatin on Day 2, wherein the C1D1 of carboplatin is about 5×(25+creatinine clearance) mg; and
   (v) administering a first dose (C1D1) of etoposide on Day 1, a second dose (C1D2) of etoposide on Day 2, and a third dose (C1D3) of etoposide on Day 3, wherein the C1D1, the C1D2, and the C1D3 of etoposide are each about 100 mg/m²;
(b) the second dosing cycle comprises:
   (i) administering a single dose (C2D1) of glofitamab on Day 8, wherein the C2D1 of glofitamab is about 30 mg;
   (ii) administering a first dose (C2D1) of rituximab on Day 1, wherein the C2D1 of rituximab is about 375 mg/m²;
   (iii) administering a single dose (C2D1) of ifosfamide on Day 2, wherein the C2D1 of ifosfamide is about 5000 mg/m² with a maximum dose of about 800 mg;
   (iv) administering a single dose (C2D1) of carboplatin on Day 2, wherein the C2D1 of carboplatin is about 5×(25+creatinine clearance) mg; and
   (v) administering a first dose (C2D1) of etoposide on Day 1, a second dose (C2D2) of etoposide on Day 2, and a third dose (C2D3) of etoposide on Day 3, wherein the C2D1, the C2D2, and the C2D3 of etoposide are each about 100 mg/m²; and
(c) the third dosing cycle comprises:
   (i) administering a single dose (C3D1) of glofitamab on Day 8, wherein the C3D1 of glofitamab is about 30 mg;
   (ii) administering a first dose (C3D1) of rituximab on Day 1, wherein the C3D1 of rituximab is about 375 mg/m²;
   (iii) administering a single dose (C3D1) of ifosfamide on Day 2, wherein the C3D1 of ifosfamide is about 5000 mg/m² with a maximum dose of about 800 mg;
   (iv) administering a single dose (C3D1) of carboplatin on Day 2, wherein the C3D1 of carboplatin is about 5×(25+creatinine clearance) mg; and
   (v) administering a first dose (C3D1) of etoposide on Day 1, a second dose (C3D2) of etoposide on Day 2, and a third dose (C3D3) of etoposide on Day 3, wherein the C3D1, the C3D2, and the C3D3 of etoposide are each about 100 mg/m².

219. The method of any one of embodiments 215 to 218, wherein mesna is administered simultaneously with any dose of ifosfamide.

220. The method of embodiment 219, wherein mesna is administered at a dose of about 5000 mg/m² intravenously.

221. The method of embodiment 220, wherein mesna is administered via continuous infusion over about 24 hours on Day 2 of each dosing cycle.

222. A method of treating a subject aged between 6 months and 17 years having a CD20-positive cell proliferative disorder comprising administering to the subject an effective amount of glofitamab, obinutuzumab, rituximab, ifosfamide, carboplatin, and etoposide in a dosing regimen comprising at least a first dosing cycle and a second dosing cycle, wherein:
(a) the first dosing cycle comprises administering a first dose (C1D1) of glofitamab on Day 8 and administering a second dose (C1D2) of glofitamab on Day 15, wherein the C1D1 of glofitamab is about 0.03 mg/kg, about 0.04 mg/kg, or about 2.5 mg, and the C1D2 of glofitamab is about 0.15 mg/kg or about 10 mg; and
(b) the second dosing cycle comprises administering a single dose (C2D1) of glofitamab on Day 8, wherein the C2D1 of glofitamab is about 0.4 mg/kg, about 0.5 mg/kg, or about 30 mg.

223. A method of treating a subject aged between 6 months and 17 years having a CD20-positive cell proliferative disorder comprising administering to the subject an effective amount of glofitamab, obinutuzumab, rituximab, ifosfamide, carboplatin, and etoposide in a dosing regimen comprising a first dosing cycle, a second dosing cycle, and a third dosing cycle, wherein:
(a) the first dosing cycle comprises administering a first dose (C1D1) of glofitamab on Day 8 and administering a second dose (C1D2) of glofitamab on Day 15, wherein the C1D1 of glofitamab is about 0.03 mg/kg, about 0.04 mg/kg, or about 2.5 mg, and the C1D2 of glofitamab is about 0.15 mg/kg or about 10 mg;
(b) the second dosing cycle comprises administering a single dose (C2D1) of glofitamab on Day 8, wherein the C2D1 of glofitamab is about 0.4 mg/kg, about 0.5 mg/kg, or about 30 mg; and
(c) the third dosing cycle comprises administering a single dose (C3D1) of glofitamab on Day 8, wherein the C3D1 of glofitamab is about 0.4 mg/kg, about 0.5 mg/kg, or about 30 mg.

224. A method of treating a subject aged between 6 months and 17 years having a CD20-positive cell proliferative disorder comprising administering to the subject an effective amount of glofitamab, obinutuzumab, rituximab, ifosfamide, carboplatin, and etoposide in a dosing regimen comprising at least a first dosing cycle and a second dosing cycle, wherein:
(a) the first dosing cycle comprises:
   (i) administering a first dose (C1D1) of glofitamab on Day 8 and a second dose (C1D2) of glofitamab on Day 15, wherein the C1D1 of glofitamab is about 0.03 mg/kg, about 0.04 mg/kg, or about 2.5 mg, and the C1D2 of glofitamab is about 0.15 mg/kg or about 10 mg;
   (ii) administering a first dose (C1D1) of obinutuzumab on Day 1 and a second dose (C1D2) of obinutuzumab on Day 2, wherein the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab, and wherein the sum of the C1D1 and the C1D2 of obinutuzumab is about 38 mg/kg, about 28 mg/kg, about 23 mg/kg, about 20 mg/kg, or about 1000 mg;
   (iii) administering a first dose (C1D1) of ifosfamide on Day 3, a second dose (C1D2) of ifosfamide on Day 4, and a third dose (C1D3) of ifosfamide on Day 5, wherein the C1D1, the C1D2, and the C1D3 of ifosfamide are each about 3000 mg/m²;
   (iv) administering a single dose (C1D1) of carboplatin on Day 3, wherein the C1D1 of carboplatin is about 635 mg/m²; and
   (v) administering a first dose (C1D1) of etoposide on Day 3, a second dose (C1D2) of etoposide on Day 4, and a third dose (C1D3) of etoposide on Day 5, wherein the C1D1, the C1D2, and the C1D3 of etoposide are each about 100 mg/m²; and
(b) the second dosing cycle comprises:
   (i) administering a single dose (C2D1) of glofitamab on Day 1, wherein the C2D1 of glofitamab is about 0.4 mg/kg, about 0.5 mg/kg, or about 30 mg;
   (ii) administering a first dose (C2D1) of rituximab on Day 5, wherein the C2D1 of rituximab is about 375 mg/m²;
   (iii) administering a first dose (C2D1) of ifosfamide on Day 6, a second dose (C2D2) of ifosfamide on Day 7, and a third dose (C2D3) of ifosfamide on Day 8, wherein the C2D1, the C2D2, and the C2D3 of ifosfamide are each about 3000 mg/m²;
   (iv) administering a single dose (C2D1) of carboplatin on Day 6, wherein the C2D1 of carboplatin is about 5×(25+creatinine clearance) mg; and
   (v) administering a first dose (C2D1) of etoposide on Day 6, a second dose (C2D2) of etoposide on Day 7, and a third dose (C2D3) of etoposide on Day 8, wherein the C2D1, the C2D2, and the C2D3 of etoposide are each about 100 mg/m².

225. A method of treating a subject aged between 6 months and 17 years having a CD20-positive cell proliferative disorder comprising administering to the subject an effective amount of glofitamab, obinutuzumab, rituximab, ifosfamide, carboplatin, and etoposide in a dosing regimen comprising a first dosing cycle, a second dosing cycle, and a third dosing cycle, wherein:
(a) the first dosing cycle comprises:
   (i) administering a first dose (C1D1) of glofitamab on Day 8 and a second dose (C1D2) of glofitamab on Day 15, wherein the C1D1 of glofitamab is about 0.03 mg/kg, about 0.04 mg/kg, or about 2.5 mg, and the C1D2 of glofitamab is about 0.15 mg/kg or about 10 mg;
   (ii) administering a first dose (C1D1) of obinutuzumab on Day 1 and a second dose (C1D2) of obinutuzumab on Day 2, wherein the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab, and wherein the sum of the C1D1 and the C1D2 of obinutuzumab is about 38 mg/kg, about 28 mg/kg, about 23 mg/kg, about 20 mg/kg, or about 1000 mg;
   (iii) administering a first dose (C1D1) of ifosfamide on Day 3, a second dose (C1D2) of ifosfamide on Day 4, and a third dose (C1D3) of ifosfamide on Day 5, wherein the C1D1, the C1D2, and the C1D3 of ifosfamide are each about 3000 mg/m²;
   (iv) administering a single dose (C1D1) of carboplatin on Day 3, wherein the C1D1 of carboplatin is about 635 mg/m²; and
   (v) administering a first dose (C1D1) of etoposide on Day 3, a second dose (C1D2) of etoposide on Day 4, and a third dose (C1D3) of etoposide on Day 5, wherein the C1D1, the C1D2, and the C1D3 of etoposide are each about 100 mg/m²;
(b) the second dosing cycle comprises:
   (i) administering a single dose (C2D1) of glofitamab on Day 1, wherein the C2D1 of glofitamab is about 0.4 mg/kg, about 0.5 mg/kg, or about 30 mg;
   (ii) administering a first dose (C2D1) of rituximab on Day 5, wherein the C2D1 of rituximab is about 375 mg/m²;
   (iii) administering a first dose (C2D1) of ifosfamide on Day 6, a second dose (C2D2) of ifosfamide on Day 7, and a third dose (C2D3) of ifosfamide on Day 8, wherein the C2D1, the C2D2, and the C2D3 of ifosfamide are each about 3000 mg/m²;
   (iv) administering a single dose (C2D1) of carboplatin on Day 6, wherein the C2D1 of carboplatin is about 5×(25+creatinine clearance) mg; and
   (v) administering a first dose (C2D1) of etoposide on Day 6, a second dose (C2D2) of etoposide on Day 7, and a third dose (C2D3) of etoposide on Day 8, wherein the C2D1, the C2D2, and the C2D3 of etoposide are each about 100 mg/m²; and
(c) the third dosing cycle comprises:
   (i) administering a single dose (C3D1) of glofitamab on Day 1, wherein the C3D1 of glofitamab is about 0.4 mg/kg, about 0.5 mg/kg, or about 30 mg;
   (ii) administering a first dose (C3D1) of rituximab on Day 5, wherein the C3D1 of rituximab is about 375 mg/m²;
   (iii) administering a first dose (C3D1) of ifosfamide on Day 6, a second dose (C3D2) of ifosfamide on Day 7, and a third dose (C3D3) of ifosfamide on Day 8, wherein the C3D1, the C3D2, and the C3D3 of ifosfamide are each about 3000 mg/m²;
   (iv) administering a single dose (C3D1) of carboplatin on Day 6, wherein the C3D1 of carboplatin is about 5×(25+creatinine clearance) mg; and
   (v) administering a first dose (C3D1) of etoposide on Day 6, a second dose (C3D2) of etoposide on Day 7, and a third dose (C3D3) of etoposide on Day 8, wherein the C3D1, the C3D2, and the C3D3 of etoposide are each about 100 mg/m².

226. The method of any one of embodiments 222 to 225, wherein mesna is administered to the subject on Days 3, 4, and 5 of the first dosing cycle, on Days 6, 7, and 8 of the second dosing cycle, and/or on Days 6, 7, and 8 of each additional dosing cycle.

227. The method of embodiment 226, wherein mesna is administered intravenously daily as five doses totaling 3000 mg/m² in amount.

228. The method of embodiment 227, wherein mesna is administered intravenously at a first dose of about 600 mg/m² prior to the administration of any dose of ifosfamide and at four repeated doses of about 600 mg/m² each at about three hours, about six hours, about nine hours, and about 12 hours, respectively, after the first dose of ifosfamide.

229. A method of treating a subject aged between 18 years and 30 years having a CD20-positive cell proliferative disorder comprising administering to the subject an effective amount of glofitamab, obinutuzumab, rituximab, ifosfamide, carboplatin, and etoposide in a dosing regimen comprising at least a first dosing cycle and a second dosing cycle, wherein:
(a) the first dosing cycle comprises administering a first dose (C1D1) of glofitamab on Day 8 and administering a second dose (C1D2) of glofitamab on Day 15, wherein the C1D1 of glofitamab is about 2.5 mg, and the C1D2 of glofitamab is about 10 mg; and
(b) the second dosing cycle comprises administering a single dose (C2D1) of glofitamab on Day 1, wherein the C2D1 of glofitamab is about 30 mg.

230. A method of treating a subject aged between 18 years and 30 years having a CD20-positive cell proliferative disorder comprising administering to the subject an effective amount of glofitamab, obinutuzumab, rituximab, ifosfamide, carboplatin, and etoposide in a dosing regimen comprising a first dosing cycle, a second dosing cycle, and a third dosing cycle, wherein:
(a) the first dosing cycle comprises administering a first dose (C1D1) of glofitamab on Day 8 and administering a second dose (C1D2) of glofitamab on Day 15, wherein the C1D1 of glofitamab is about 2.5 mg, and the C1D2 of glofitamab is about 10 mg;
(b) the second dosing cycle comprises administering a single dose (C2D1) of glofitamab on Day 1, wherein the C2D1 of glofitamab is about 30 mg; and
(c) the third dosing cycle comprises administering a single dose (C3D1) of glofitamab on Day 1, wherein the C3D1 of glofitamab is about 30 mg.

231. A method of treating a subject aged between 18 years and 30 years having a CD20-positive cell proliferative disorder comprising administering to the subject an effective amount of glofitamab, obinutuzumab, rituximab, ifosfamide, carboplatin, and etoposide in a dosing regimen comprising at least a first dosing cycle and a second dosing cycle, wherein:
(a) the first dosing cycle comprises:
   (i) administering a first dose (C1D1) of glofitamab on Day 8 and a second dose (C1D2) of glofitamab on Day 15, wherein the C1D1 of glofitamab is about 2.5 mg, and the C1D2 of glofitamab is about 10 mg;
   (ii) administering a first dose (C1D1) of obinutuzumab on Day 1 and a second dose (C1D2) of obinutuzumab on Day 2, wherein the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab, and wherein the sum of the C1D1 and the C1D2 of obinutuzumab is about 1000 mg;
   (iii) administering a single dose (C1D1) of ifosfamide on Day 3, wherein the C1D1 of ifosfamide is about 5000 mg/m² with a maximum dose of about 800 mg;
   (iv) administering a single dose (C1D1) of carboplatin on Day 3, wherein the C1D1 of carboplatin is about 5×(25+creatinine clearance) mg; and
   (v) administering a first dose (C1D1) of etoposide on Day 3, a second dose (C1D2) of etoposide on Day 4, and a third dose (C1D3) of etoposide on Day 5, wherein the C1D1, the C1D2, and the C1D3 of etoposide are each about 100 mg/m²; and
(b) the second dosing cycle comprises:
   (i) administering a single dose (C2D1) of glofitamab on Day 1, wherein the C2D1 of glofitamab is about 30 mg;
   (ii) administering a first dose (C2D1) of rituximab on Day 5, wherein the C2D1 of rituximab is about 375 mg/m²;
   (iii) administering a single dose (C2D1) of ifosfamide on Day 6, wherein the C2D1 of ifosfamide is about 5000 mg/m² with a maximum dose of about 800 mg;
   (iv) administering a single dose (C2D1) of carboplatin on Day 6, wherein the C2D1 of carboplatin is about 5×(25+creatinine clearance) mg; and
   (v) administering a first dose (C2D1) of etoposide on Day 6, a second dose (C2D2) of etoposide on Day 7, and a third dose (C2D3) of etoposide on Day 8, wherein the C2D1, the C2D2, and the C2D3 of etoposide are each about 100 mg/m².

232. A method of treating a subject aged between 18 years and 30 years having a CD20-positive cell proliferative disorder comprising administering to the subject an effective amount of glofitamab, obinutuzumab, rituximab, ifosfamide, carboplatin, and etoposide in a dosing regimen comprising a first dosing cycle, a second dosing cycle, and a third dosing cycle, wherein:
(a) the first dosing cycle comprises:
   (i) administering a first dose (C1D1) of glofitamab on Day 8 and a second dose (C1D2) of glofitamab on Day 15, wherein the C1D1 of glofitamab is about 2.5 mg, and the C1D2 of glofitamab is about 10 mg;
   (ii) administering a first dose (C1D1) of obinutuzumab on Day 1 and a second dose (C1D2) of obinutuzumab on Day 2, wherein the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab, and wherein the sum of the C1D1 and the C1D2 of obinutuzumab is about 1000 mg;
   (iii) administering a single dose (C1D1) of ifosfamide on Day 3, wherein the C1D1 of ifosfamide is about 5000 mg/m² with a maximum dose of about 800 mg;
   (iv) administering a single dose (C1D1) of carboplatin on Day 3, wherein the C1D1 of carboplatin is about 5×(25+creatinine clearance) mg; and
   (v) administering a first dose (C1D1) of etoposide on Day 3, a second dose (C1D2) of etoposide on Day 4, and a third dose (C1D3) of etoposide on Day 5, wherein the C1D1, the C1D2, and the C1D3 of etoposide are each about 100 mg/m²;
(b) the second dosing cycle comprises:
   (i) administering a single dose (C2D1) of glofitamab on Day 1, wherein the C2D1 of glofitamab is about 30 mg;
   (ii) administering a first dose (C2D1) of rituximab on Day 5, wherein the C2D1 of rituximab is about 375 mg/m²;
   (iii) administering a single dose (C2D1) of ifosfamide on Day 6, wherein the C2D1 of ifosfamide is about 5000 mg/m² with a maximum dose of about 800 mg;
   (iv) administering a single dose (C2D1) of carboplatin on Day 6, wherein the C2D1 of carboplatin is about 5×(25+creatinine clearance) mg; and
   (v) administering a first dose (C2D1) of etoposide on Day 6, a second dose (C2D2) of etoposide on Day 7, and a third dose (C2D3) of etoposide on Day 8, wherein the C2D1, the C2D2, and the C2D3 of etoposide are each about 100 mg/m²; and
(c) the third dosing cycle comprises:
   (i) administering a single dose (C3D1) of glofitamab on Day 1, wherein the C3D1 of glofitamab is about 30 mg;
   (ii) administering a first dose (C3D1) of rituximab on Day 5, wherein the C3D1 of rituximab is about 375 mg/m²;
   (iii) administering a single dose (C3D1) of ifosfamide on Day 6, wherein the C3D1 of ifosfamide is about 5000 mg/m² with a maximum dose of about 800 mg;
   (iv) administering a single dose (C3D1) of carboplatin on Day 6, wherein the C3D1 of carboplatin is about 5×(25+creatinine clearance) mg; and
   (v) administering a first dose (C3D1) of etoposide on Day 6, a second dose (C3D2) of etoposide on Day 7, and a third dose (C3D3) of etoposide on Day 8, wherein the C3D1, the C3D2, and the C3D3 of etoposide are each about 100 mg/m².

233. The method of any one of embodiments 229 to 232, wherein mesna is administered simultaneously with any dose of ifosfamide.

234. The method of embodiment 233, wherein mesna is administered intravenously at a dose of about 5000 mg/m².

235. The method of embodiment 234, wherein mesna is administered via continuous infusion over about 24 hours on Day 3 of the first dosing cycle, on Day 6 of the second dosing cycle, and/or on Day 6 of each additional dosing cycle.

236. The method of any one of embodiments 215 to 221, wherein the CD20-positive cell proliferative disorder is a relapsed and/or refractory DLBCL.

237. The method of any one of embodiments 222 to 235, wherein the CD20-positive cell proliferative disorder is a relapsed and/or refractory mature B cell NHL.

238. A bispecific antibody that binds to CD20 and CD3 for use in a method of treating a subject having a CD20-positive cell proliferative disorder, wherein the bispecific antibody that binds to CD20 and CD3 is to be administered in combination with an anti-CD20 antibody and one or more chemotherapeutic agents selected from ifosfamide, carboplatin, and/or etoposide
in a dosing regimen comprising at least a first dosing cycle and a second dosing cycle.

239. Use of a bispecific antibody that binds to CD20 and CD3 in the manufacture of a medicament for the treatment of a subject having a CD20-positive cell proliferative disorder, wherein in said treatment the bispecific antibody that binds to CD20 and CD3 is to be administered in combination with an anti-CD20 antibody and one or more chemotherapeutic agents selected from ifosfamide, carboplatin, and/or etoposide
in a dosing regimen comprising at least a first dosing cycle and a second dosing cycle.

240. Use of a bispecific antibody that binds to CD20 and CD3 for treating a subject having a CD20-positive cell proliferative disorder, wherein the bispecific antibody that binds to CD20 and CD3 is to be administered in combination with an anti-CD20 antibody and one or more chemotherapeutic agents selected from ifosfamide, carboplatin, and/or etoposide
in a dosing regimen comprising at least a first dosing cycle and a second dosing cycle.

241. The bispecific antibody for use or use of any one of embodiments 238-240, wherein the subject is aged 18 years or older.

242. The bispecific antibody for use or use of embodiment 241, wherein the subject is aged 31 years or older.

243. The bispecific antibody for use or use of any one of embodiments 238-242, wherein the first dosing cycle comprises a first dose (C1D1) of the bispecific antibody and a second dose (C1D2) of the bispecific antibody, wherein the C1D1 of the bispecific antibody is about 2.5 mg, and the C1D2 of the bispecific antibody is about 10 mg; and
the second dosing cycle comprises a single dose (C2D1) of the bispecific antibody, wherein the C2D1 of the bispecific antibody is about 10 mg, about 16 mg, or about 30 mg.

244. The bispecific antibody for use or use of embodiment 243, wherein the C2D1 of the bispecific antibody is about 30 mg.

245. The bispecific antibody for use or use of embodiment 243 or 244, wherein the C1D1 of the bispecific antibody and the C1D2 of the bispecific antibody are to be administered to the subject on Days 8 and 15, respectively, of the first dosing cycle.

246. The bispecific antibody for use or use of any one of embodiments 243 to 245, wherein the C2D1 of the bispecific antibody is to be administered to the subject on Day 8 of the second dosing cycle.

247. The bispecific antibody for use or use of any one of embodiments 238 to 246, wherein the anti-CD20 antibody is obinutuzumab and/or rituximab.

248. The bispecific antibody for use or use of embodiment 247, wherein the first dosing cycle comprises a single dose (C1D1) of obinutuzumab; and the second dosing cycle comprises a single dose (C2D1) of rituximab.

249. The bispecific antibody for use or use of embodiment 248, wherein the C1D1 of obinutuzumab is about 1000 mg and the C2D1 of rituximab is about 375 mg/m².

250. The bispecific antibody for use or use of embodiment 248 or 249, wherein the anti-CD20 antibody is to be administered in a dosing regimen comprising at least a first dosing cycle and a second dosing cycle, wherein the first dosing cycle comprises the C1D1 of obinutuzumab to be administered to the subject on Day 1; and the second dosing cycle comprises the C2D1 of rituximab to be administered to the subject on Day 1.

251. The bispecific antibody for use or use of any one of embodiments 238 to 250, wherein the bispecific antibody that binds to CD20 and CD3 is to be administered in combination with an anti-CD20 antibody, ifosfamide, carboplatin, and etoposide.

252. The bispecific antibody for use or use of embodiment 251, wherein the first dosing cycle comprises a single dose (C1D1) of ifosfamide; a single dose (C1D1) of carboplatin; and a first dose (C1D1) of etoposide, a second dose (C1D2) of etoposide, and a third dose (C1D3) of etoposide; and the second cycle comprises a single dose (C2D1) of ifosfamide; a single dose (C2D1) of carboplatin; and a first dose (C2D1) of etoposide, a second dose (C2D2) of etoposide, and a third dose (C2D3) of etoposide.

253. The bispecific antibody for use or use of embodiment 251 or 252, wherein ifosfamide is to be administered at a dose of about 5000 mg/m², about 4000 mg/m², or about 1666 mg/m², carboplatin is to be administered at a dose in mg to target area under the curve (AUC) of about 5 mg/mL/min with maximum dose of about 750 mg, and etoposide is to be administered at a dose of about 100 mg/m² or about 75 mg/m².

254. The bispecific antibody for use or use of embodiment 253, wherein ifosfamide is to be administered at a dose of about 5000 mg/m², carboplatin is to be administered at a dose in mg to target area under the curve (AUC) of about 5 mg/mL/min with maximum dose of about 750 mg and etoposide is to be administered at a dose of about 100 mg/m².

255. The bispecific antibody for use or use of embodiment 251, wherein carboplatin is to be administered at a dose of about 5×(25+CreatinineClearance(CrCl)) mg with maximum dose of about 750 mg.

256. The bispecific antibody for use or use of embodiment 255, wherein
(a) the subject is male, and wherein CrCl is calculated using the formula CrCl=([140−age]×[weight in kg])/(72×[serum creatinine in mg/dL]); or
(b) the subject is female, and wherein CrCl is calculated using the formula CrCl=0.85×([140−age]×[weight in kg])/(72×[serum creatinine in mg/dL]).

257. The bispecific antibody for use or use of any one of embodiments 252 to 256, wherein ifosfamide and carboplatin are to be administered on Day 2 of the first and second dosing cycles and the C1D1-C1D3 and C2D1-C2D3 of etoposide are to be administered on Days 1, 2, and 3, respectively, of the first and second dosing cycles.

258. The bispecific antibody for use or use of any one of embodiments 238 to 257, wherein the first and second dosing cycles are each 21-day dosing cycles.

259. The bispecific antibody for use or use of any one of embodiments 238 to 258, wherein the dosing regimen comprises one or more additional dosing cycles.

260. The bispecific antibody for use or use of embodiment 259, wherein the one or more additional dosing cycles are each 21-day dosing cycles.

261. The bispecific antibody for use or use of embodiment 259 or 260, wherein the dosing regimen comprises three dosing cycles in total.

262. The bispecific antibody for use or use of any one of embodiments 259 to 261, wherein the one or more additional dosing cycles each comprises:
(a) an additional single dose of the bispecific antibody that binds to CD20 and CD3,
(b) an additional single dose of the anti-CD20 antibody, and
(c) an additional single dose of ifosfamide, an additional single dose of carboplatin and an additional first dose, an additional second dose, and an additional third dose of etoposide.

263. The bispecific antibody for use or use of embodiment 262, wherein the additional single dose of the bispecific antibody is about 30 mg.

264. The bispecific antibody for use or use of embodiment 262 or 263, wherein the additional single dose of the bispecific antibody is to be administered to the subject on Day 8 of each of the one or more additional dosing cycles.

265. The bispecific antibody for use or use of any one of embodiments 262 to 264, wherein the anti-CD20 antibody is rituximab.

266. The bispecific antibody for use or use of embodiment 265, wherein the additional single dose of rituximab is about 375 mg/m².

267. The bispecific antibody for use or use of embodiment 265 or 266, wherein the additional single dose of rituximab is to be administered on Day 1 of each of the one or more additional dosing cycles.

268. The bispecific antibody for use or use of any one of embodiments 262 to 267, wherein the additional single dose of ifosfamide is about 5000 mg/m², about 4000 mg/m², or 1 about 666 mg/m², the additional single dose of carboplatin is in mg to target area under the curve (AUC) of 5 mg/mL/min with maximum dose of about 750 mg and the additional first dose, the additional second dose, and the additional third dose of etoposide are each about 100 mg/m² or about 75 mg/m².

269. The bispecific antibody for use or use of embodiment 268, wherein ifosfamide is to be administered at a dose of 5000 mg/m², carboplatin is to be administered at a dose in mg to target area under the curve (AUC) of 5 mg/mL/min with maximum dose of about 750 mg and etoposide is to be administered at a dose of about 100 mg/m².

270. The bispecific antibody for use or use of any one of embodiments 262-267, wherein the additional single dose of carboplatin is about 5×(25+CreatinineClearance(CrCl)) mg with maximum dose of about 750 mg.

271. The bispecific antibody for use or use of embodiment 270, wherein
(a) the subject is male, and wherein CrCl is calculated using the formula CrCl=([140−age]×[weight in kg])/(72×[serum creatinine in mg/dL]); or
(b) the subject is female, and wherein CrCl is calculated using the formula CrCl=0.85×([140−age]×[weight in kg])/(72×[serum creatinine in mg/dL]).

272. The bispecific antibody for use or use of any one of embodiments 259 to 271, wherein ifosfamide and carboplatin are to be administered on Day 2 of each of the one or more additional dosing cycles and the additional first dose, the additional second dose, and the additional third dose of etoposide are to be administered on Days 1, 2, and 3, respectively, of each of the one or more additional dosing cycles.

273. The bispecific antibody for use or use of any one of embodiments 238 to 272, wherein the bispecific antibody that binds to CD20 and CD3 is to be administered in combination with one or more additional therapeutic agents.

274. The bispecific antibody for use or use of embodiment 273, wherein the one or more additional therapeutic agent is tocilizumab.

275. The bispecific antibody for use or use of embodiment 274, wherein the weight of the subject is greater than or equal to about 30 kg and tocilizumab is to be administered at a dose of about 8 mg/kg or the weight of the subject is less than 30 kg and tocilizumab is to be administered at a dose of about 12 mg/kg, and wherein the maximum dose is about 800 mg.

276. The bispecific antibody for use or use of embodiment 273, wherein the one or more additional therapeutic agents is a corticosteroid.

277. The bispecific antibody for use or use of embodiment 276, wherein the corticosteroid comprises prednisone, prednisolone, methylprednisolone, or dexamethasone.

278. The bispecific antibody for use or use of embodiment 277, wherein the corticosteroid is dexamethasone.

279. The bispecific antibody for use or use of embodiment 278, wherein dexamethasone is to be administered intravenously at a dose of about 20 mg at least about one hour prior to the administration of any dose of the bispecific antibody.

280. The bispecific antibody for use or use of embodiment 279, wherein dexamethasone is to be administered intravenously at a dose of about 20 mg or about 80 mg at least about one hour prior to the administration of any dose of obinutuzumab.

281. The bispecific antibody for use or use of embodiment 277, wherein the corticosteroid is methylprednisolone.

282. The bispecific antibody for use or use of embodiment 281, wherein methylprednisolone is administered intravenously at a dose of about 80 mg at least about one hour prior to the administration of any dose of the bispecific antibody.

283. The bispecific antibody for use or use of embodiment 281, wherein methylprednisolone is administered intravenously at a dose of about 80 mg at least about one hour prior to the administration of any dose of obinutuzumab.

284. The bispecific antibody for use or use of embodiment 277, wherein the corticosteroid is prednisone.

285. The bispecific antibody for use or use of embodiment 284, wherein prednisone is administered orally at a dose of about 100 mg at least about one hour prior to the administration of any dose of the bispecific antibody.

286. The bispecific antibody for use or use of embodiment 277, wherein the corticosteroid is prednisolone.

287. The bispecific antibody for use or use of embodiment 286, wherein prednisolone is administered intravenously at a dose of about 100 mg at least about one hour prior to the administration of any dose of the bispecific antibody.

288. The bispecific antibody for use or use of embodiment 273, wherein the one or more additional therapeutic agents is an antihistamine.

289. The bispecific antibody for use or use of embodiment 288, wherein the antihistamine is diphenhydramine.

290. The bispecific antibody for use or use of embodiment 289, wherein diphenhydramine is to be administered orally or intravenously at a dose of about 50 mg at least about 30 minutes prior to the administration of any dose of the bispecific antibody.

291. The bispecific antibody for use or use of embodiment 273, wherein the one or more additional therapeutic agents comprises G-CSF.

292. The bispecific antibody for use or use of embodiment 291, wherein G-CSF is to be administered between about one day and about two days after administration of any dose of rituximab, ifosfamide, carboplatin, and/or etoposide.

293. The bispecific antibody for use or use of embodiment 273, wherein the one or more additional therapeutic agents is an antipyretic.

294. The bispecific antibody for use or use of embodiment 293, wherein the antipyretic is acetaminophen or paracetamol.

295. The bispecific antibody for use or use of embodiment 294, wherein acetaminophen or paracetamol is to be administered orally or intravenously at a dose of between about 500 to about 1000 mg at least about 30 minutes prior to the administration of any dose of the bispecific antibody.

296. The bispecific antibody for use or use of embodiment 294, wherein acetaminophen or paracetamol is to be administered orally or intravenously at a dose of between about 500 to about 1000 mg at least about 30 minutes prior to the administration of any dose of obinutuzumab.

297. The bispecific antibody for use or use of embodiment 273, wherein the one or more additional therapeutic agents is mesna.

298. The bispecific antibody for use or use of embodiment 297, wherein mesna is to be administered at a dose of about 5000 mg/m², about 4000 mg/m², or about 1666 mg/m² intravenously.

299. The bispecific antibody for use or use of embodiment 298, wherein mesna is to be administered via continuous infusion over about 24 hours on Day 2 of each dosing cycle.

300. The bispecific antibody for use or use of embodiment 298 or 299, wherein mesna is to be administered simultaneously with any dose of ifosfamide.

301. A bispecific antibody that binds to CD20 and CD3 for use in a method of treating a subject aged between 6 months and 17 years having a CD20-positive cell proliferative disorder, wherein the bispecific antibody that binds to CD20 and CD3 is to be administered in combination with an anti-CD20 antibody and one or more chemotherapeutic agents selected from ifosfamide, carboplatin, and/or etoposide
in a dosing regimen comprising at least a first dosing cycle and a second dosing cycle.

302. Use of a bispecific antibody that binds to CD20 and CD3 in the manufacture of a medicament for the treatment of a subject aged between 6 months and 17 years having a CD20-positive cell proliferative disorder, wherein in said treatment the bispecific antibody that binds to CD20 and CD3 is to be administered in combination with an anti-CD20 antibody and one or more chemotherapeutic agents selected from ifosfamide, carboplatin, and/or etoposide
in a dosing regimen comprising at least a first dosing cycle and a second dosing cycle.

303. Use of a bispecific antibody that binds to CD20 and CD3 for treating a subject aged between 6 months and 17 years having a CD20-positive cell proliferative disorder, wherein the bispecific antibody that binds to CD20 and CD3 is to be administered in combination with an anti-CD20 antibody and one or more chemotherapeutic agents selected from ifosfamide, carboplatin, and/or etoposide
in a dosing regimen comprising at least a first dosing cycle and a second dosing cycle.

304. The bispecific antibody for use or use of any one of embodiments 301-303, wherein
the first dosing cycle comprises a first dose (C1D1) of the bispecific antibody and a second dose (C1D2) of the bispecific antibody, wherein the C1D1 of the bispecific antibody is about 0.03 mg/kg, about 0.04 mg/kg, or about 2.5 mg, and the C1D2 of the bispecific antibody is about 0.15 mg/kg or about 10 mg; and
the second dosing cycle comprises a single dose (C2D1) of the bispecific antibody, wherein the C2D1 of the bispecific antibody is about 0.4 mg/kg, about 0.5 mg/kg, or about 30 mg.

305. The bispecific antibody for use or use of embodiment 304, wherein:
(a) the subject's body weight is greater than or equal to about 7.5 kg and less than about 13 kg, and wherein the C1D1 of the bispecific antibody is about 0.04 mg/kg, the C1D2 of the bispecific antibody is about 0.15 mg/kg, and the C2D1 of the bispecific antibody is about 0.5 mg/kg;
(b) the subject's body weight is greater than or equal to about 13 kg and less than about 45 kg, and wherein the C1D1 of the bispecific antibody is about 0.03 mg/kg, the C1D2 of the bispecific antibody is about 0.15 mg/kg, and the C2D1 of the bispecific antibody is about 0.4 mg/kg; or
(c) the subject's body weight is greater than or equal to about 45 kg, and wherein the C1D1 of the bispecific antibody is about 2.5 mg, the C1D2 of the bispecific antibody is about 10 mg, and the C2D1 of the bispecific antibody is about 30 mg.

306. The bispecific antibody for use or use of embodiment 304 or 305, wherein the C1D1 of the bispecific antibody and the C1D2 of the bispecific antibody are to be administered to the subject on Days 8 and 15, respectively, of the first dosing cycle.

307. The bispecific antibody for use or use of any one of embodiments 304 to 306, wherein the C2D1 of the bispecific antibody is to be administered to the subject on Day 1 of the second dosing cycle.

308. The bispecific antibody for use or use of any one of embodiments 301 to 307, wherein the anti-CD20 antibody is obinutuzumab and/or rituximab.

309. The bispecific antibody for use or use of embodiment 308, wherein the first dosing cycle comprises a first dose (C1D1) of obinutuzumab and a second dose (C1D2) of obinutuzumab.

310. The bispecific antibody for use or use of embodiment 309, wherein:
(a) the subject's body weight is greater than or equal to about 7.5 kg and less than about 13 kg, and wherein the sum of the C1D1 and the C1D2 of obinutuzumab is about 38 mg/kg;
(b) the subject's body weight is greater than or equal to about 13 kg and less than about 20 kg, and wherein the sum of the C1D1 and the C1D2 of obinutuzumab is about 28 mg/kg;
(c) the subject's body weight is greater than or equal to about 20 kg and less than about 32 kg, and wherein the sum of the C1D1 and the C1D2 of obinutuzumab is about 23 mg/kg;
(d) the subject's body weight is greater than or equal to about 32 kg and less than about 45 kg, and wherein the sum of the C1D1 and the C1D2 of obinutuzumab is about 20 mg/kg; or
(e) the subject's body weight is greater than or equal to about 45 kg, and wherein the sum of the C1D1 and the C1D2 of obinutuzumab is about 1000 mg.

311. The bispecific antibody for use or use of embodiment 309 or 310, wherein the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab.

312. The bispecific antibody for use or use of any one of embodiments 309 to 311, wherein:
(a) the subject's body weight is greater than or equal to about 7.5 kg and less than about 13 kg, and wherein the C1D1 of obinutuzumab is about 3.8 mg/kg and the C1D2 of obinutuzumab is about 34.2 mg/kg;
(b) the subject's body weight is greater than or equal to about 13 kg and less than about 20 kg, and wherein the C1D1 of obinutuzumab is about 2.8 mg/kg and the C1D2 of obinutuzumab is about 35.2 mg/kg;
(c) the subject's body weight is greater than or equal to about 20 kg and less than about 32 kg, and wherein the C1D1 of obinutuzumab is about 2.3 mg/kg and the C1D2 of obinutuzumab is about 35.7 mg/kg;
(d) the subject's body weight is greater than or equal to about 32 kg and less than about 45 kg, and wherein the C1D1 of obinutuzumab is about 2.0 mg/kg and the C1D2 of obinutuzumab is about 36.0 mg/kg; or
(e) the subject's body weight is greater than or equal to about 45 kg, and wherein the C1D1 of obinutuzumab is about 100 mg and the C1D2 of obinutuzumab is about 900 mg.

313. The bispecific antibody for use or use of any one of embodiments 309 to 312, wherein the C1D1 of obinutuzumab is to be administered to the subject on Day 1 of the first dosing cycle and the C1D2 of obinutuzumab is to be administered to the subject on Day 2 of the first dosing cycle.

314. The bispecific antibody for use or use of embodiment 309, wherein the second dosing cycle comprises a single dose (C2D1) of rituximab.

315. The bispecific antibody for use or use of embodiment 314, wherein the C2D1 of rituximab is about 375 mg/m².

316. The bispecific antibody for use or use of embodiment 314 or 315, wherein the C2D1 of rituximab is to be administered to the subject on Day 5 of the second dosing cycle.

317. The bispecific antibody for use or use of any one of embodiments 291 to 304, wherein the bispecific antibody that binds to CD20 and CD3 is to be administered in combination with an anti-CD20 antibody, ifosfamide, carboplatin, and etoposide.

318. The bispecific antibody for use or use of embodiment 317, wherein the first dosing cycle comprises:
(a) a first dose (C1D1) of ifosfamide, a second dose (C1D2) of ifosfamide, and a third dose (C1D3) of ifosfamide;
(b) a single dose (C1D1) of carboplatin; and
(c) a first dose (C1D1) of etoposide, a second dose (C1D2) of etoposide, and a third dose (C1D3) of etoposide;
and the second cycle comprises:
(a) a first dose (C2D1) of ifosfamide, a second dose (C2D2) of ifosfamide, and a third dose (C2D3) of ifosfamide;
(b) a single dose (C2D1) of carboplatin; and
(c) a first dose (C2D1) of etoposide, a second dose (C2D2) of etoposide, and a third dose (C2D3) of etoposide.

319. The bispecific antibody for use or use of embodiment 317 or 318, wherein ifosfamide is to be administered at a dose of about 3000 mg/m² for each dose of ifosfamide, carboplatin is to be administered at a dose of about 635 mg/m², and etoposide is to be administered at a dose of about 100 mg/m² for each dose of etoposide.

320. The bispecific antibody for use or use of embodiment 318 or 319, wherein:
(a) the C1D1, C1D2, and C1D3 of ifosfamide are to be administered on Days 3, 4, and 5, respectively, of the first dosing cycle;
(b) the C1D1 of carboplatin is to be administered on Day 3 of the first dosing cycle;
(c) the C1D1, C1D2, and C1D3 of etoposide are to be administered on Days 3, 4, and 5, respectively, of the first dosing cycle;
(d) the C2D1, C2D2, and C2D3 of ifosfamide are to be administered on Days 6, 7, and 8, respectively, of the second dosing cycle;
(e) the C2D1 of carboplatin is to be administered on Day 6 of the second dosing cycle; and
(f) the C2D1, C2D2, and C2D3 of etoposide are to be administered on Days 6, 7, and 8, respectively, of the second dosing cycle.

321. The bispecific antibody for use or use of any one of embodiments 301 to 320, wherein the first and second dosing cycles are each 21-day dosing cycles.

322. The bispecific antibody for use or use of any one of embodiments 301 to 321, wherein the dosing regimen comprises one or more additional dosing cycles.

323. The bispecific antibody for use or use of embodiment 322, wherein the one or more additional dosing cycles are each 21-day dosing cycles.

324. The bispecific antibody for use or use of embodiment 322 or 323, wherein the dosing regimen comprises three dosing cycles in total.

325. The bispecific antibody for use or use of any one of embodiments 322 to 324, wherein the one or more additional dosing cycles each comprises:
(a) an additional single dose of the bispecific antibody that binds to CD20 and CD3,
(b) an additional single dose of the anti-CD20 antibody, and
(c) an additional first dose, an additional second dose, and an additional third dose of ifosfamide; an additional single dose of carboplatin; and an additional first dose, an additional second dose, and an additional third dose of etoposide.

326. The bispecific antibody for use or use of embodiment 325, wherein:
(a) the subject's body weight is greater than or equal to about 7.5 kg and less than about 13 kg, and wherein the additional single dose of the bispecific antibody is about 0.5 mg/kg;
(b) the subject's body weight is greater than or equal to about 13 kg and less than about 45 kg, and wherein the additional single dose of the bispecific antibody is about 0.4 mg/kg; or
(c) the subject's body weight is greater than or equal to about 45 kg, and wherein the additional single dose of the bispecific antibody is about 30 mg.

327. The bispecific antibody for use or use of embodiment 325 or 326, wherein the additional single dose of the bispecific antibody is to be administered to the subject on Day 1 of each of the one or more additional dosing cycles.

328. The bispecific antibody for use or use of any one of embodiments 325 to 327, wherein the anti-CD20 antibody is rituximab.

329. The bispecific antibody for use or use of embodiment 328, wherein the additional single dose of rituximab is about 375 mg/m².

330. The bispecific antibody for use or use of embodiment 328 or 329, wherein the additional single dose of rituximab is to be administered on Day 5 of each of the one or more additional dosing cycles.

331. The bispecific antibody for use or use of any one of embodiments 322 to 330, wherein the additional first dose, additional second dose, and additional third dose of ifosfamide are each about 3000 mg/m², the additional single dose of carboplatin is about 635 mg/m², and the additional first dose, the additional second dose, and the additional third dose of etoposide are each about 100 mg/m².

332. The bispecific antibody for use or use of any one of embodiments 322 to 331, wherein:
(a) the additional first dose, the additional second dose, and the additional third dose of ifosfamide are to be administered to the subject on Days 6, 7, and 8, respectively, of each of the one or more additional dosing cycles;
(b) the additional single dose of carboplatin is to be administered on Day 6 of each of the one or more additional dosing cycles; and
(c) the additional first dose, the additional second dose, and the additional third dose of etoposide are to be administered to the subject on Days 6, 7, and 8, respectively, of each of the one or more additional dosing cycles.

333. The bispecific antibody for use or use of any one of embodiments 301 to 332, wherein the bispecific antibody that binds to CD20 and CD3 is to be administered in combination with one or more additional therapeutic agents.

334. The bispecific antibody for use or use of embodiment 333, wherein the one or more additional therapeutic agent is tocilizumab.

335. The bispecific antibody for use or use of embodiment 334, wherein the weight of the subject is greater than or equal to about 30 kg and tocilizumab is to be administered at a dose of about 8 mg/kg or the weight of the subject is less than 30 kg and tocilizumab is to be administered at a dose of about 12 mg/kg, and wherein the maximum dose is about 800 mg.

336. The bispecific antibody for use or use of embodiment 333, wherein the one or more additional therapeutic agents is a corticosteroid.

337. The bispecific antibody for use or use of embodiment 336, wherein the corticosteroid comprises prednisone, prednisolone, methylprednisolone, or dexamethasone.

338. The bispecific antibody for use or use of embodiment 337, wherein the corticosteroid is dexamethasone.

339. The bispecific antibody for use or use of embodiment 338, wherein dexamethasone is to be administered intravenously at a dose of between about 0.15 mg/kg to about 0.5 mg/kg at least about one hour prior to the administration of any dose of the bispecific antibody, and wherein the maximum daily dose is 10 mg.

340. The bispecific antibody for use or use of embodiment 338, wherein dexamethasone is to be administered intravenously at a dose of between about 0.15 mg/kg to about 0.5 mg/kg at least about one hour prior to the administration of any dose of obinutuzumab, and wherein the maximum daily dose is 10 mg.

341. The bispecific antibody for use or use of embodiment 337, wherein the corticosteroid is methylprednisolone.

342. The bispecific antibody for use or use of embodiment 341, wherein methylprednisolone is to be administered intravenously at a dose of between about 1 mg/kg to about 2 mg/kg at least about one hour prior to the administration of any dose of the bispecific antibody.

343. The bispecific antibody for use or use of embodiment 341, wherein methylprednisolone is to be administered intravenously at a dose of between about 1 mg/kg to about 2 mg/kg at least about one hour prior to the administration of any dose of obinutuzumab.

344. The bispecific antibody for use or use of embodiment 337, wherein the corticosteroid is prednisone or prednisolone.

345. The bispecific antibody for use or use of embodiment 344, wherein prednisone or prednisolone is administered intravenously at a dose of about 100 mg or about 2 mg/kg at least about one hour prior to the administration of any dose of the bispecific antibody.

346. The bispecific antibody for use or use of embodiment 344, wherein prednisone or prednisolone is administered intravenously at a dose of about 100 mg or about 2 mg/kg at least about one hour prior to the administration of any dose of obinutuzumab.

347. The bispecific antibody for use or use of embodiment 333, wherein the one or more additional therapeutic agents is an antihistamine.

348. The bispecific antibody for use or use of embodiment 347, wherein the antihistamine is diphenhydramine.

349. The bispecific antibody for use or use of embodiment 348, wherein the subject is aged between two years and 17 years, and wherein diphenhydramine is to be administered intravenously at a dose of between about 10 mg to 20 mg with a maximum single dose of about 1.25 mg/kg.

350. The bispecific antibody for use or use of embodiment 348, wherein the subject is aged less than two years, and wherein diphenhydramine is to be administered rectally at a dose of about 20 mg.

351. The bispecific antibody for use or use of embodiment 349 or 350, wherein diphenhydramine is to be administered at least about 30 minutes prior to the administration of any dose of the bispecific antibody and/or the anti-CD20 antibody.

352. The bispecific antibody for use or use of embodiment 333, wherein the one or more additional therapeutic agents comprises G-CSF.

353. The bispecific antibody for use or use of embodiment 352, wherein G-CSF is to be administered between about one day and about two days after administration of any dose of rituximab, ifosfamide, carboplatin, and/or etoposide.

354. The bispecific antibody for use or use of embodiment 353, wherein G-CSF is to be administered intravenously or subcutaneously at a dose of about 5 μg/kg/day or about 10 μg/kg/day.

355. The bispecific antibody for use or use of embodiment 354, wherein G-CSF is to be administered at a dose of about 5 μg/kg/day in the first dosing cycle and about 10 μg/kg/day in the second dosing cycle and/or each additional dosing cycle.

356. The bispecific antibody for use or use of embodiment 333, wherein the one or more additional therapeutic agents is an antipyretic.

357. The bispecific antibody for use or use of embodiment 356, wherein the antipyretic is acetaminophen or paracetamol.

358. The bispecific antibody for use or use of embodiment 357, wherein acetaminophen or paracetamol is to be administered orally or intravenously at a dose of between about 500 to about 1000 mg.

359. The bispecific antibody for use or use of embodiment 358, wherein acetaminophen or paracetamol is to be administered at least about 30 minutes prior to the administration of any dose of the bispecific antibody and/or the anti-CD20 antibody.

360. The bispecific antibody for use or use of embodiment 333, wherein the one or more additional therapeutic agents is mesna.

361. The bispecific antibody for use or use of embodiment 360, wherein mesna is to be administered intravenously daily as five doses totaling 3000 mg/m² in amount.

362. The bispecific antibody for use or use of embodiment 361, wherein mesna is to be administered intravenously at a first dose of about 600 mg/m² prior to the administration of any dose of ifosfamide and at four repeated doses of about 600 mg/m² each at about three hours, about six hours, about nine hours, and about 12 hours, respectively, after the first dose of ifosfamide.

363. The bispecific antibody for use or use of embodiment 361 or 362, wherein mesna is to be administered daily to the subject on Days 3, 4, and 5 of the first dosing cycle, on Days 6, 7, and 8 of the second dosing cycle, and/or on Days 6, 7, and 8 of each additional dosing cycle.

364. A bispecific antibody that binds to CD20 and CD3 for use in a method of treating a subject aged between 18 years and 30 years having a CD20-positive cell proliferative disorder, wherein the bispecific antibody that binds to CD20 and CD3 is to be administered in combination with an anti-CD20 antibody and one or more chemotherapeutic agents selected from ifosfamide, carboplatin, and/or etoposide
in a dosing regimen comprising at least a first dosing cycle and a second dosing cycle.

365. Use of a bispecific antibody that binds to CD20 and CD3 in the manufacture of a medicament for the treatment of a subject aged between 18 years and 30 years having a CD20-positive cell proliferative disorder, wherein in said treatment the bispecific antibody that binds to CD20 and CD3 is to be administered in combination with an anti-CD20 antibody and one or more chemotherapeutic agents selected from ifosfamide, carboplatin, and/or etoposide
in a dosing regimen comprising at least a first dosing cycle and a second dosing cycle.

366. Use of a bispecific antibody that binds to CD20 and CD3 for treating a subject aged between 18 years and 30 years having a CD20-positive cell proliferative disorder, wherein the bispecific antibody that binds to CD20 and CD3 is to be administered in combination with an anti-CD20 antibody and one or more chemotherapeutic agents selected from ifosfamide, carboplatin, and/or etoposide
in a dosing regimen comprising at least a first dosing cycle and a second dosing cycle.

367. The bispecific antibody for use or use of any one of embodiments 364-366, wherein
the first dosing cycle comprises a first dose (C1D1) of the bispecific antibody and a second dose (C1D2) of the bispecific antibody, wherein the C1D1 of the bispecific antibody is about 2.5 mg, and the C1D2 of the bispecific antibody is about 10 mg; and
the second dosing cycle comprises a single dose (C2D1) of the bispecific antibody, wherein the C2D1 of the bispecific antibody is about 30 mg.

368. The bispecific antibody for use or use of embodiment 367, wherein the C1D1 of the bispecific antibody and the C1D2 of the bispecific antibody are to be administered to the subject on Days 8 and 15, respectively, of the first dosing cycle.

369. The bispecific antibody for use or use of embodiment 367 or 368, wherein the C2D1 of the bispecific antibody is to be administered to the subject on Day 1 of the second dosing cycle.

370. The bispecific antibody for use or use of any one of embodiments 364 to 369, wherein the anti-CD20 antibody is obinutuzumab and/or rituximab.

371. The bispecific antibody for use or use of embodiment 370, wherein the first dosing cycle comprises a first dose (C1D1) of obinutuzumab and a second dose (C1D2) of obinutuzumab.

372. The bispecific antibody for use or use of embodiment 371, the sum of the C1D1 and the C1D2 of obinutuzumab is about 1000 mg.

373. The bispecific antibody for use or use of embodiment 371 or 372, wherein the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab.

374. The bispecific antibody for use or use of any one of embodiments 371 to 373, wherein the C1D1 of obinutuzumab is about 100 mg and the C1D2 of obinutuzumab is about 900 mg.

375. The bispecific antibody for use or use of any one of embodiments 371 to 374, wherein the C1D1 of obinutuzumab is to be administered to the subject on Day 1 of the first dosing cycle and the C1D2 of obinutuzumab is to be administered to the subject on Day 2 of the first dosing cycle.

376. The bispecific antibody for use or use of embodiment 370, wherein the second dosing cycle comprises a single dose (C2D1) of rituximab.

377. The bispecific antibody for use or use of embodiment 376, wherein the C2D1 of rituximab is about 375 mg/m².

378. The bispecific antibody for use or use of embodiment 376 or 377, wherein the C2D1 of rituximab is to be administered to the subject on Day 5 of the second dosing cycle.

379. The bispecific antibody for use or use of any one of embodiments 364 to 378, wherein the bispecific antibody that binds to CD20 and CD3 is to be administered in combination with an anti-CD20 antibody, ifosfamide, carboplatin, and etoposide.

380. The bispecific antibody for use or use of embodiment 379, wherein the first dosing cycle comprises:
(a) a single dose (C1D1) of ifosfamide;
(b) a single dose (C1D1) of carboplatin; and
(c) a first dose (C1D1) of etoposide, a second dose (C1D2) of etoposide, and a third dose (C1D3) of etoposide;
and the second cycle comprises:
(a) a single dose (C2D1) of ifosfamide;
(b) a single dose (C2D1) of carboplatin; and
(c) a first dose (C2D1) of etoposide, a second dose (C2D2) of etoposide, and a third dose (C2D3) of etoposide.

381. The bispecific antibody for use or use of embodiment 379 or 380, wherein ifosfamide is to be administered at a dose of about 5000 mg/m², carboplatin is to be administered at a dose of about 5×(25+CreatinineClearance (CrCl)) mg with maximum dose of about 750 mg, and etoposide is to be administered at a dose of about 100 mg/m² for each dose of etoposide.

382. The bispecific antibody for use or use of embodiment 381, wherein:
(a) the subject is male, and wherein CrCl is calculated using the formula CrCl=([140−age]×[weight in kg])/(72×[serum creatinine in mg/dL]); or
(b) the subject is female, and wherein CrCl is calculated using the formula CrCl=0.85×([140−age]×[weight in kg])/(72×[serum creatinine in mg/dL]).

383. The bispecific antibody for use or use of embodiment 381 or 382, wherein:
(a) the subject has CrCl<about 60 mL/min, and wherein each single dose of ifosfamide is reduced to 4000 mg/m²; and/or
(b) the subject has CrCl<about 50 mL/min, and wherein each dose of etoposide is reduced to about 75 mg/m².

384. The bispecific antibody for use or use of any one of embodiments 380 to 383, wherein:
(a) the C1D1 of ifosfamide is to be administered on Day 3 of the first dosing cycle;
(b) the C1D1 of carboplatin is to be administered on Day 3 of the first dosing cycle;
(c) the C1D1, C1D2, and C1D3 of etoposide are to be administered on Days 3, 4, and 5, respectively, of the first dosing cycle;
(d) the C2D1 of ifosfamide is to be administered on Day 6 of the second dosing cycle;
(e) the C2D1 of carboplatin is to be administered on Day 6 of the second dosing cycle; and
(f) the C2D1, C2D2, and C2D3 of etoposide are to be administered on Days 6, 7, and 8, respectively, of the second dosing cycle.

385. The bispecific antibody for use or use of any one of embodiments 364 to 384, wherein the first and second dosing cycles are each 21-day dosing cycles.

386. The bispecific antibody for use or use of any one of embodiments 364 to 385, wherein the dosing regimen comprises one or more additional dosing cycles.

387. The bispecific antibody for use or use of embodiment 386, wherein the one or more additional dosing cycles are each 21-day dosing cycles.

388. The bispecific antibody for use or use of embodiment 386 or 387, wherein the dosing regimen comprises three dosing cycles in total.

389. The bispecific antibody for use or use of any one of embodiments 386 to 388, wherein the one or more additional dosing cycles each comprises:
(a) an additional single dose of the bispecific antibody that binds to CD20 and CD3,
(b) an additional single dose of the anti-CD20 antibody, and
(c) an additional single dose of ifosfamide, an additional single dose of carboplatin and an additional first dose, an additional second dose, and an additional third dose of etoposide.

390. The bispecific antibody for use or use of embodiment 389, wherein the additional single dose of the bispecific antibody is about 30 mg.

391. The bispecific antibody for use or use of embodiment 390, wherein the additional single dose of the bispecific antibody is to be administered to the subject on Day 1 of each of the one or more additional dosing cycles.

392. The bispecific antibody for use or use of any one of embodiments 389 to 391, wherein the anti-CD20 antibody is rituximab.

393. The bispecific antibody for use or use of embodiment 392, wherein the additional single dose of rituximab is about 375 mg/m².

394. The bispecific antibody for use or use of embodiment 392 or 393, wherein the additional single dose of rituximab is to be administered on Day 5 of each of the one or more additional dosing cycles.

395. The bispecific antibody for use or use of any one of embodiments 375 to 380, wherein the additional single dose of ifosfamide is about 5000 mg/m², the additional single dose of carboplatin is about 5×(25+CreatinineClearance (CrCl)) mg with maximum dose of about 750 mg, and the additional first dose, the additional second dose, and the additional third dose of etoposide are each about 100 mg/m².

396. The bispecific antibody for use or use of embodiment 395, wherein:
(a) the subject is male, and wherein CrCl is calculated using the formula CrCl=([140−age]×[weight in kg])/(72×[serum creatinine in mg/dL]); or
(b) the subject is female, and wherein CrCl is calculated using the formula CrCl=0.85×([140−age]×[weight in kg])/(72×[serum creatinine in mg/dL]).

397. The bispecific antibody for use or use of embodiment 395 or 396, wherein:
(a) the subject has CrCl<about 60 mL/min, and wherein the additional single dose of ifosfamide is reduced to 4000 mg/m²; and/or
(b) the subject has CrCl<about 50 mL/min, and wherein each additional dose of etoposide is reduced to about 75 mg/m².

398. The bispecific antibody for use or use of any one of embodiments 389 to 397, wherein:
(a) the additional single dose of ifosfamide is to be administered on Day 6 of each of the one or more additional dosing cycles;
(b) the additional single dose of carboplatin is to be administered on Day 6 of each of the one or more additional dosing cycles; and
(c) the additional first dose, the additional second dose, and the additional third dose of etoposide are to be administered to the subject on Days 6, 7, and 8, respectively, of each of the one or more additional dosing cycles.

399. The bispecific antibody for use or use of any one of embodiments 364 to 398, wherein the bispecific antibody that binds to CD20 and CD3 is to be administered in combination with one or more additional therapeutic agents.

400. The bispecific antibody for use or use of embodiment 399, wherein the one or more additional therapeutic agent is tocilizumab.

401. The bispecific antibody for use or use of embodiment 400, wherein the weight of the subject is greater than or equal to about 30 kg and tocilizumab is to be administered at a dose of about 8 mg/kg or the weight of the subject is less than 30 kg and tocilizumab is to be administered at a dose of about 12 mg/kg, and wherein the maximum dose is about 800 mg.

402. The bispecific antibody for use or use of embodiment 399, wherein the one or more additional therapeutic agents is a corticosteroid.

403. The bispecific antibody for use or use of embodiment 402, wherein the corticosteroid comprises prednisone, prednisolone, methylprednisolone, or dexamethasone.

404. The bispecific antibody for use or use of embodiment 403, wherein the corticosteroid is dexamethasone.

405. The bispecific antibody for use or use of embodiment 404, wherein dexamethasone is to be administered intravenously at a dose of between about 0.15 mg/kg to about 0.5 mg/kg at least about one hour prior to the administration of any dose of the bispecific antibody, and wherein the maximum daily dose is 10 mg.

406. The bispecific antibody for use or use of embodiment 404, wherein dexamethasone is to be administered intravenously at a dose of between about 0.15 mg/kg to about 0.5 mg/kg at least about one hour prior to the administration of any dose of obinutuzumab, and wherein the maximum daily dose is 10 mg.

407. The bispecific antibody for use or use of embodiment 403, wherein the corticosteroid is methylprednisolone.

408. The bispecific antibody for use or use of embodiment 407, wherein methylprednisolone is to be administered intravenously at a dose of between about 1 mg/kg to about 2 mg/kg at least about one hour prior to the administration of any dose of the bispecific antibody.

409. The bispecific antibody for use or use of embodiment 407, wherein methylprednisolone is to be administered intravenously at a dose of between about 1 mg/kg to about 2 mg/kg at least about one hour prior to the administration of any dose of obinutuzumab.

410. The bispecific antibody for use or use of embodiment 399, wherein the one or more additional therapeutic agents is an antihistamine.

411. The bispecific antibody for use or use of embodiment 410, wherein the antihistamine is diphenhydramine.

412. The bispecific antibody for use or use of embodiment 411, wherein diphenhydramine is to be administered orally or intravenously at a dose of about 50 mg.

413. The bispecific antibody for use or use of embodiment 412, wherein diphenhydramine is to be administered at least about 30 minutes prior to the administration of any dose of the bispecific antibody and/or the anti-CD20 antibody.

414. The bispecific antibody for use or use of embodiment 399, wherein the one or more additional therapeutic agents comprises G-CSF.

415. The bispecific antibody for use or use of embodiment 414, wherein G-CSF is to be administered between about one day and about two days after administration of any dose of rituximab, ifosfamide, carboplatin, and/or etoposide.

416. The bispecific antibody for use or use of embodiment 415, wherein G-CSF is to be administered intravenously or subcutaneously at a dose of about 5 μg/kg/day or about 10 μg/kg/day.

417. The bispecific antibody for use or use of embodiment 415, wherein G-CSF is to be administered at a dose of about 5 μg/kg/day in the first dosing cycle and about 10 μg/kg/day in the second dosing cycle and/or each additional dosing cycle.

418. The bispecific antibody for use or use of embodiment 399, wherein the one or more additional therapeutic agents is an antipyretic.

419. The bispecific antibody for use or use of embodiment 418, wherein the antipyretic is acetaminophen or paracetamol.

420. The bispecific antibody for use or use of embodiment 419, wherein acetaminophen or paracetamol is to be administered orally or intravenously at a dose of between about 500 to about 1000 mg.

421. The bispecific antibody for use or use of embodiment 420, wherein acetaminophen or paracetamol is to be administered at least about 30 minutes prior to the administration of any dose of the bispecific antibody and/or the anti-CD20 antibody.

422. The bispecific antibody for use or use of embodiment 399, wherein the one or more additional therapeutic agents is mesna.

423. The bispecific antibody for use or use of embodiment 422, wherein mesna is to be administered intravenously at a dose of about 5000 mg/m².

424. The bispecific antibody for use or use of embodiment 423, wherein mesna is to be administered via continuous infusion over about 24 hours on Day 3 of the first dosing cycle, on Day 6 of the second dosing cycle, and/or on Day 6 of each additional dosing cycle.

425. The bispecific antibody for use or use of embodiment 423 or 424, wherein mesna is to be administered simultaneously with any dose of ifosfamide.

426. The bispecific antibody for use or use of any one of embodiments 238 to 425, wherein the bispecific antibody comprises at least one Fab molecule which specifically binds to CD20 comprising the following six hypervariable regions (HVRs):
(i) an HVR-H1 comprising the amino acid sequence of YSWIN (SEQ ID NO: 1);
(ii) an HVR-H2 comprising the amino acid sequence of RIFPGDGDTDYNGKFKG (SEQ ID NO: 2);
(iii) an HVR-H3 comprising the amino acid sequence of NVFDGYWLVY (SEQ ID NO:3);
(iv) an HVR-L1 comprising the amino acid sequence of RSSKSLLHSNGITYLY (SEQ ID NO: 4);
(v) an HVR-L2 comprising the amino acid sequence of QMSNLVS (SEQ ID NO: 5); and
(vi) an HVR-L3 comprising the amino acid sequence of AQNLELPYT (SEQ ID NO: 6).

427. The bispecific antibody for use or use of any one of embodiments 238 to 412, wherein the bispecific antibody comprises at least one Fab molecule which specifically binds to CD20 comprising (a) a heavy chain variable (VH) domain comprising an amino acid sequence having at least 95% sequence identity to the amino acid sequence of SEQ ID NO: 7; (b) a light chain variable (VL) domain comprising an amino acid sequence having at least 95% sequence identity to the amino acid sequence of SEQ ID NO: 8; or (c) a VH domain as in (a) and a VL domain as in (b).

428. The bispecific antibody for use or use of embodiment 427, wherein the Fab molecule which specifically binds to CD20 comprises (a) a VH domain comprising the amino acid sequence of SEQ ID NO: 7 and (b) a VL domain comprising the amino acid sequence of SEQ ID NO: 8.

429. The bispecific antibody for use or use of any one of embodiments 238 to 428, wherein the bispecific antibody comprises at least one Fab molecule which specifically binds to CD3 comprising the following six HVRs:
(i) an HVR-H1 comprising the amino acid sequence of TYAMN (SEQ ID NO: 9);
(ii) an HVR-H2 comprising the amino acid sequence of RIRSKYNNYATYYADSVKG (SEQ ID NO: 10);
(iii) an HVR-H3 comprising the amino acid sequence of HGNFGNSYVSWFAY (SEQ ID NO: 11);
(iv) an HVR-L1 comprising the amino acid sequence of GSSTGAVTTSNYAN (SEQ ID NO: 12);
(v) an HVR-L2 comprising the amino acid sequence of GTNKRAP (SEQ ID NO: 13); and
(vi) an HVR-L3 comprising the amino acid sequence of ALWYSNLWV (SEQ ID NO: 14).

430. The bispecific antibody for use or use of any one of embodiments 238 to 429, wherein the bispecific antibody comprises at least one Fab molecule which specifically binds to CD3 comprising (a) a VH domain comprising an amino acid sequence having at least 95% sequence identity to the amino acid sequence of SEQ ID NO: 15; (b) a VL domain comprising an amino acid sequence having at least 95% sequence identity to the amino acid sequence of SEQ ID NO: 16; or (c) a VH domain as in (a) and a VL domain as in (b).

431. The bispecific antibody for use or use of embodiment 430, wherein the Fab molecule which specifically binds to CD3 comprises (a) a VH domain comprising the amino acid sequence of SEQ ID NO: 15 and (b) a VL domain comprising the amino acid sequence of SEQ ID NO: 16.

432. The bispecific antibody for use or use of any one of embodiments 238 to 431, wherein the bispecific antibody is bivalent for CD20 and monovalent for CD3.

433. The bispecific antibody for use or use of any one of embodiments 238 to 432, wherein the bispecific antibody comprises two Fab molecules which specifically bind to CD20 and one Fab molecule which specifically binds to CD3.

434. The bispecific antibody for use or use of any one of embodiments 238 to 433, wherein the bispecific antibody is a humanized antibody.

435. The bispecific antibody for use or use of any one of embodiments 238 to 434, wherein the bispecific antibody is glofitamab.

436. The bispecific antibody for use or use of any one of embodiments 238 to 435, wherein the bispecific antibody is to be administered intravenously.

437. The bispecific antibody for use or use of any one of embodiments 238 to 436, wherein the anti-CD20 antibody is to be administered intravenously.

438. The bispecific antibody for use or use of any one of embodiments 238 to 437, wherein ifosfamide, carboplatin, and/or etoposide are to be administered intravenously.

439. The bispecific antibody for use or use of any one of embodiments 238 to 438, wherein the CD20-positive cell proliferative disorder is a B cell proliferative disorder.

440. The bispecific antibody for use or use of embodiment 439, wherein the B cell proliferative disorder is a non-Hodgkin's lymphoma (NHL) or a central nervous system lymphoma (CNSL).

441. The bispecific antibody for use or use of embodiment 440, wherein the NHL is a diffuse-large B cell lymphoma (DLBCL), a follicular lymphoma (FL), a mantle cell lymphoma (MCL), a marginal zone lymphoma (MZL), a high-grade B cell lymphoma, a primary mediastinal (thymic) large B cell lymphoma (PMLBCL), a diffuse B cell lymphoma, or a small lymphocytic lymphoma.

442. The bispecific antibody for use or use of embodiment 440, wherein the NHL is a Burkitt lymphoma (BL) or a Burkitt leukemia (BAL).

443. The bispecific antibody for use or use of any one of embodiments 440 to 442, wherein the NHL is relapsed and/or refractory.

444. The bispecific antibody for use or use of any one of embodiments 440 to 443, wherein the NHL is aggressive and/or mature.

445. The bispecific antibody for use or use of embodiment 439, wherein the B cell proliferative disorder is a relapsed and/or refractory mature B cell NHL.

446. The bispecific antibody for use or use of embodiment 443, wherein the subject has received one prior systemic therapy.

447. The bispecific antibody for use or use of embodiment 446, wherein the subject has received no more than one prior systemic therapy.

448. The bispecific antibody for use or use of embodiment 446 or 447, wherein the prior systemic therapy comprises an anti-CD20 antibody and an anthracycline.

449. The bispecific antibody for use or use of any one of embodiments 238 to 448, wherein the subject is human.

450. The bispecific antibody for use or use of any one of embodiments 238 to 449, wherein the subject is transplant or CAR-T cell therapy eligible.

451. The bispecific antibody for use or use of embodiment 450, wherein the subject receives autologous stem cell transplantation (ASCT) after completion of the dosing regimen of any one of embodiments 238 to 449.

452. The bispecific antibody for use or use of embodiment 451, wherein the ASCT is an autologous hematopoietic stem cell transplant.

453. The bispecific antibody for use or use of embodiment 450, wherein the subject receives allogenic hematopoietic stem cell transplantation after completion of the dosing regimen of any one of embodiments 237 to 448.

454. The bispecific antibody for use or use of embodiment 450, wherein the subject receives CAR-T cell therapy after completion of the dosing regimen of any one of embodiments 238 to 449.

455. Glofitamab for use in a method of treating a subject having a CD20-positive cell proliferative disorder, wherein glofitamab is to be administered in combination with obinutuzumab, rituximab, ifosfamide, carboplatin, and etoposide in a dosing regimen comprising at least a first dosing cycle and a second dosing cycle, wherein
(a) the first dosing cycle comprises a first dose (C1D1) of glofitamab to be administered on Day 8 and a second dose (C1D2) of glofitamab to be administered on Day 15, wherein the C1D1 of glofitamab is about 2.5 mg, and the C1D2 of glofitamab is about 10 mg; and
(b) the second dosing cycle comprises a single dose (C2D1) of glofitamab to be administered on Day 8, wherein the C2D1 of glofitamab is about 30 mg.

456. Use of glofitamab in the manufacture of a medicament for the treatment of a subject having a CD20-positive cell proliferative disorder, wherein in said treatment glofitamab is to be administered in combination with obinutuzumab, rituximab, ifosfamide, carboplatin, and etoposide in a dosing regimen comprising at least a first dosing cycle and a second dosing cycle, wherein
(a) the first dosing cycle comprises a first dose (C1D1) of glofitamab to be administered on Day 8 and a second dose (C1D2) of glofitamab to be administered on Day 15, wherein the C1D1 of glofitamab is about 2.5 mg, and the C1D2 of glofitamab is about 10 mg; and
(b) the second dosing cycle comprises a single dose (C2D1) of glofitamab to be administered on Day 8, wherein the C2D1 of glofitamab is about 30 mg.

457. Use of glofitamab for treating a subject having a CD20-positive cell proliferative disorder, wherein glofitamab is to be administered in combination with obinutuzumab, rituximab, ifosfamide, carboplatin, and etoposide in a dosing regimen comprising at least a first dosing cycle and a second dosing cycle, wherein
(a) the first dosing cycle comprises a first dose (C1D1) of glofitamab to be administered on Day 8 and a second dose (C1D2) of glofitamab to be administered on Day 15, wherein the C1D1 of glofitamab is about 2.5 mg, and the C1D2 of glofitamab is about 10 mg; and
(b) the second dosing cycle comprises a single dose (C2D1) of glofitamab to be administered on Day 8, wherein the C2D1 of glofitamab is about 30 mg.

458. Glofitamab for use in a method of treating a subject having a CD20-positive cell proliferative disorder, wherein glofitamab is to be administered in combination with obinutuzumab, rituximab, ifosfamide, carboplatin, and etoposide in a dosing regimen comprising a first dosing cycle, a second dosing cycle, and a third dosing cycle, wherein
(a) the first dosing cycle comprises a first dose (C1D1) of glofitamab to be administered on Day 8 and a second dose (C1D2) of glofitamab to be administered on Day 15, wherein the C1D1 of glofitamab is about 2.5 mg, and the C1D2 of glofitamab is about 10 mg;
(b) the second dosing cycle comprises a single dose (C2D1) of glofitamab to be administered on Day 8, wherein the C2D1 of glofitamab is about 30 mg; and
(c) the third dosing cycle comprises a single dose (C3D1) of glofitamab to be administered on Day 8, wherein the C3D1 of glofitamab is about 30 mg.

459. Use of glofitamab in the manufacture of a medicament for the treatment of a subject having a CD20-positive cell proliferative disorder, wherein in said treatment glofitamab is to be administered in combination with obinutuzumab, rituximab, ifosfamide, carboplatin, and etoposide in a dosing regimen comprising a first dosing cycle, a second dosing cycle, and a third dosing cycle, wherein
(a) the first dosing cycle comprises a first dose (C1D1) of glofitamab to be administered on Day 8 and a second dose (C1D2) of glofitamab to be administered on Day 15, wherein the C1D1 of glofitamab is about 2.5 mg, and the C1D2 of glofitamab is about 10 mg;
(b) the second dosing cycle comprises a single dose (C2D1) of glofitamab to be administered on Day 8, wherein the C2D1 of glofitamab is about 30 mg; and
(c) the third dosing cycle comprises a single dose (C3D1) of glofitamab to be administered on Day 8, wherein the C3D1 of glofitamab is about 30 mg.

460. Use of glofitamab for treating a subject having a CD20-positive cell proliferative disorder, wherein glofitamab is to be administered in combination with obinutuzumab, rituximab, ifosfamide, carboplatin, and etoposide in a dosing regimen comprising a first dosing cycle, a second dosing cycle, and a third dosing cycle, wherein
(a) the first dosing cycle comprises a first dose (C1D1) of glofitamab to be administered on Day 8 and a second dose (C1D2) of glofitamab to be administered on Day 15, wherein the C1D1 of glofitamab is about 2.5 mg, and the C1D2 of glofitamab is about 10 mg;
(b) the second dosing cycle comprises a single dose (C2D1) of glofitamab to be administered on Day 8, wherein the C2D1 of glofitamab is about 30 mg; and
(c) the third dosing cycle comprises a single dose (C3D1) of glofitamab to be administered on Day 8, wherein the C3D1 of glofitamab is about 30 mg.

461. Glofitamab for use in a method of treating a subject having a CD20-positive cell proliferative disorder, wherein glofitamab is to be administered in combination with obinutuzumab, rituximab, ifosfamide, carboplatin, and etoposide in a dosing regimen comprising at least a first dosing cycle and a second dosing cycle, wherein
(a) the first dosing cycle comprises:
   (i) a first dose (C1D1) of glofitamab to be administered on Day 8 and a second dose (C1D2) of glofitamab to be administered on Day 15, wherein the C1D1 of glofitamab is about 2.5 mg, and the C1D2 of glofitamab is about 10 mg;
   (ii) a first dose (C1D1) of obinutuzumab to be administered on Day 1, wherein the C1D1 of obinutuzumab is about 1000 mg;
   (iii) a single dose (C1D1) of ifosfamide to be administered on Day 2, wherein the C1D1 of ifosfamide is about 5000 mg/m² with a maximum dose of about 800 mg;
   (iv) a single dose (C1D1) of carboplatin to be administered on Day 2, wherein the C1D1 of carboplatin is about 5×(25+creatinine clearance) mg; and
   (v) a first dose (C1D1) of etoposide to be administered on Day 1, a second dose (C1D2) of etoposide to be administered on Day 2, and a third dose (C1D3) of etoposide to be administered on Day 3, wherein the C1D1, the C1D2, and the C1D3 of etoposide are each about 100 mg/m²; and
(b) the second dosing cycle comprises:
   (i) a single dose (C2D1) of glofitamab to be administered on Day 8, wherein the C2D1 of glofitamab is about 30 mg;
   (ii) a first dose (C2D1) of rituximab to be administered on Day 1, wherein the C2D1 of rituximab is about 375 mg/m²;
   (iii) a single dose (C2D1) of ifosfamide to be administered on Day 2, wherein the C2D1 of ifosfamide is about 5000 mg/m² with a maximum dose of about 800 mg;
   (iv) a single dose (C2D1) of carboplatin to be administered on Day 2, wherein the C2D1 of carboplatin is about 5×(25+creatinine clearance) mg; and
   (v) a first dose (C2D1) of etoposide to be administered on Day 1, a second dose (C2D2) of etoposide to be administered on Day 2, and a third dose (C1D3) of etoposide to be administered on Day 3, wherein the C2D1, the C2D2, and the C2D3 of etoposide are each about 100 mg/m².

462. Use of glofitamab in the manufacture of a medicament for the treatment of a subject having a CD20-positive cell proliferative disorder, wherein in said treatment glofitamab is to be administered in combination with obinutuzumab, rituximab, ifosfamide, carboplatin, and etoposide in a dosing regimen comprising at least a first dosing cycle and a second dosing cycle, wherein
(a) the first dosing cycle comprises:
   (i) a first dose (C1D1) of glofitamab to be administered on Day 8 and a second dose (C1D2) of glofitamab to be administered on Day 15, wherein the C1D1 of glofitamab is about 2.5 mg, and the C1D2 of glofitamab is about 10 mg;
   (ii) a first dose (C1D1) of obinutuzumab to be administered on Day 1, wherein the C1D1 of obinutuzumab is about 1000 mg;
   (iii) a single dose (C1D1) of ifosfamide to be administered on Day 2, wherein the C1D1 of ifosfamide is about 5000 mg/m² with a maximum dose of about 800 mg;
   (iv) a single dose (C1D1) of carboplatin to be administered on Day 2, wherein the C1D1 of carboplatin is about 5×(25+creatinine clearance) mg; and
   (v) a first dose (C1D1) of etoposide to be administered on Day 1, a second dose (C1D2) of etoposide to be administered on Day 2, and a third dose (C1D3) of etoposide to be administered on Day 3, wherein the C1D1, the C1D2, and the C1D3 of etoposide are each about 100 mg/m²; and
(b) the second dosing cycle comprises:
   (i) a single dose (C2D1) of glofitamab to be administered on Day 8, wherein the C2D1 of glofitamab is about 30 mg;
   (ii) a first dose (C2D1) of rituximab to be administered on Day 1, wherein the C2D1 of rituximab is about 375 mg/m²;
   (iii) a single dose (C2D1) of ifosfamide to be administered on Day 2, wherein the C2D1 of ifosfamide is about 5000 mg/m² with a maximum dose of about 800 mg;
   (iv) a single dose (C2D1) of carboplatin to be administered on Day 2, wherein the C2D1 of carboplatin is about 5×(25+creatinine clearance) mg; and
   (v) a first dose (C2D1) of etoposide to be administered on Day 1, a second dose (C2D2) of etoposide to be administered on Day 2, and a third dose (C1D3) of etoposide to be administered on Day 3, wherein the C2D1, the C2D2, and the C2D3 of etoposide are each about 100 mg/m².

463. Use of glofitamab for treating a subject having a CD20-positive cell proliferative disorder, wherein glofitamab is to be administered in combination with obinutuzumab, rituximab, ifosfamide, carboplatin, and etoposide in a dosing regimen comprising at least a first dosing cycle and a second dosing cycle, wherein
(a) the first dosing cycle comprises:
   (i) a first dose (C1D1) of glofitamab to be administered on Day 8 and a second dose (C1D2) of glofitamab to be administered on Day 15, wherein the C1D1 of glofitamab is about 2.5 mg, and the C1D2 of glofitamab is about 10 mg;
   (ii) a first dose (C1D1) of obinutuzumab to be administered on Day 1, wherein the C1D1 of obinutuzumab is about 1000 mg;
   (iii) a single dose (C1D1) of ifosfamide to be administered on Day 2, wherein the C1D1 of ifosfamide is about 5000 mg/m² with a maximum dose of about 800 mg;
   (iv) a single dose (C1D1) of carboplatin to be administered on Day 2, wherein the C1D1 of carboplatin is about 5×(25+creatinine clearance) mg; and
   (v) a first dose (C1D1) of etoposide to be administered on Day 1, a second dose (C1D2) of etoposide to be administered on Day 2, and a third dose (C1D3) of etoposide to be administered on Day 3, wherein the C1D1, the C1D2, and the C1D3 of etoposide are each about 100 mg/m²; and
(b) the second dosing cycle comprises:
   (i) a single dose (C2D1) of glofitamab to be administered on Day 8, wherein the C2D1 of glofitamab is about 30 mg;
   (ii) a first dose (C2D1) of rituximab to be administered on Day 1, wherein the C2D1 of rituximab is about 375 mg/m²;
   (iii) a single dose (C2D1) of ifosfamide to be administered on Day 2, wherein the C2D1 of ifosfamide is about 5000 mg/m² with a maximum dose of about 800 mg;
   (iv) a single dose (C2D1) of carboplatin to be administered on Day 2, wherein the C2D1 of carboplatin is about 5×(25+creatinine clearance) mg; and
   (v) a first dose (C2D1) of etoposide to be administered on Day 1, a second dose (C2D2) of etoposide to be administered on Day 2, and a third dose (C1D3) of etoposide to be administered on Day 3, wherein the C2D1, the C2D2, and the C2D3 of etoposide are each about 100 mg/m².

464. Glofitamab for use in a method of treating a subject having a CD20-positive cell proliferative disorder, wherein glofitamab is to be administered in combination with obinutuzumab, rituximab, ifosfamide, carboplatin, and etoposide in a dosing regimen comprising a first dosing cycle, a second dosing cycle, and a third dosing cycle, wherein
(a) the first dosing cycle comprises:
   (i) a first dose (C1D1) of glofitamab to be administered on Day 8 and a second dose (C1D2) of glofitamab to be administered on Day 15, wherein the C1D1 of glofitamab is about 2.5 mg, and the C1D2 of glofitamab is about 10 mg;
   (ii) a first dose (C1D1) of obinutuzumab to be administered on Day 1, wherein the C1D1 of obinutuzumab is about 1000 mg;
   (iii) a single dose (C1D1) of ifosfamide to be administered on Day 2, wherein the C1D1 of ifosfamide is about 5000 mg/m² with a maximum dose of about 800 mg;
   (iv) a single dose (C1D1) of carboplatin to be administered on Day 2, wherein the C1D1 of carboplatin is about 5×(25+creatinine clearance) mg; and
   (v) a first dose (C1D1) of etoposide to be administered on Day 1, a second dose (C1D2) of etoposide to be administered on Day 2, and a third dose (C1D3) of etoposide to be administered on Day 3, wherein the C1D1, the C1D2, and the C1D3 of etoposide are each about 100 mg/m²;
(b) the second dosing cycle comprises:
   (i) a single dose (C2D1) of glofitamab to be administered on Day 8, wherein the C2D1 of glofitamab is about 30 mg;
   (ii) a first dose (C2D1) of rituximab to be administered on Day 1, wherein the C2D1 of rituximab is about 375 mg/m²;
   (iii) a single dose (C2D1) of ifosfamide to be administered on Day 2, wherein the C2D1 of ifosfamide is about 5000 mg/m² with a maximum dose of about 800 mg;
   (iv) a single dose (C2D1) of carboplatin to be administered on Day 2, wherein the C2D1 of carboplatin is about 5×(25+creatinine clearance) mg; and
   (v) a first dose (C2D1) of etoposide to be administered on Day 1, a second dose (C2D2) of etoposide to be administered on Day 2, and a third dose (C2D3) of etoposide to be administered on Day 3, wherein the C2D1, the C2D2, and the C2D3 of etoposide are each about 100 mg/m²; and
(c) the third dosing cycle comprises:
   (i) a single dose (C3D1) of glofitamab to be administered on Day 8, wherein the C3D1 of glofitamab is about 30 mg;
   (ii) a first dose (C3D1) of rituximab to be administered on Day 1, wherein the C3D1 of rituximab is about 375 mg/m²;
   (iii) a single dose (C3D1) of ifosfamide to be administered on Day 2, wherein the C3D1 of ifosfamide is about 5000 mg/m² with a maximum dose of about 800 mg;
   (iv) a single dose (C3D1) of carboplatin to be administered on Day 2, wherein the C3D1 of carboplatin is about 5×(25+creatinine clearance) mg; and
   (v) a first dose (C3D1) of etoposide to be administered on Day 1, a second dose (C3D2) of etoposide to be administered on Day 2, and a third dose (C3D3) of etoposide to be administered on Day 3, wherein the C3D1, the C3D2, and the C3D3 of etoposide are each about 100 mg/m².

465. Use of glofitamab in the manufacture of a medicament for the treatment of a subject having a CD20-positive cell proliferative disorder, wherein in said treatment glofitamab is to be administered in combination with obinutuzumab, rituximab, ifosfamide, carboplatin, and etoposide in a dosing regimen comprising a first dosing cycle, a second dosing cycle, and a third dosing cycle, wherein
(a) the first dosing cycle comprises:
   (i) a first dose (C1D1) of glofitamab to be administered on Day 8 and a second dose (C1D2) of glofitamab to be administered on Day 15, wherein the C1D1 of glofitamab is about 2.5 mg, and the C1D2 of glofitamab is about 10 mg;
   (ii) a first dose (C1D1) of obinutuzumab to be administered on Day 1, wherein the C1D1 of obinutuzumab is about 1000 mg;
   (iii) a single dose (C1D1) of ifosfamide to be administered on Day 2, wherein the C1D1 of ifosfamide is about 5000 mg/m² with a maximum dose of about 800 mg;
   (iv) a single dose (C1D1) of carboplatin to be administered on Day 2, wherein the C1D1 of carboplatin is about 5×(25+creatinine clearance) mg; and
   (v) a first dose (C1D1) of etoposide to be administered on Day 1, a second dose (C1D2) of etoposide to be administered on Day 2, and a third dose (C1D3) of etoposide to be administered on Day 3, wherein the C1D1, the C1D2, and the C1D3 of etoposide are each about 100 mg/m²;
(b) the second dosing cycle comprises:
   (i) a single dose (C2D1) of glofitamab to be administered on Day 8, wherein the C2D1 of glofitamab is about 30 mg;
   (ii) a first dose (C2D1) of rituximab to be administered on Day 1, wherein the C2D1 of rituximab is about 375 mg/m²;
   (iii) a single dose (C2D1) of ifosfamide to be administered on Day 2, wherein the C2D1 of ifosfamide is about 5000 mg/m² with a maximum dose of about 800 mg;
   (iv) a single dose (C2D1) of carboplatin to be administered on Day 2, wherein the C2D1 of carboplatin is about 5×(25+creatinine clearance) mg; and
   (v) a first dose (C2D1) of etoposide to be administered on Day 1, a second dose (C2D2) of etoposide to be administered on Day 2, and a third dose (C2D3) of etoposide to be administered on Day 3, wherein the C2D1, the C2D2, and the C2D3 of etoposide are each about 100 mg/m²; and
(c) the third dosing cycle comprises:
   (i) a single dose (C3D1) of glofitamab to be administered on Day 8, wherein the C3D1 of glofitamab is about 30 mg;
   (ii) a first dose (C3D1) of rituximab to be administered on Day 1, wherein the C3D1 of rituximab is about 375 mg/m²;
   (iii) a single dose (C3D1) of ifosfamide to be administered on Day 2, wherein the C3D1 of ifosfamide is about 5000 mg/m² with a maximum dose of about 800 mg;
   (iv) a single dose (C3D1) of carboplatin to be administered on Day 2, wherein the C3D1 of carboplatin is about 5×(25+creatinine clearance) mg; and
   (v) a first dose (C3D1) of etoposide to be administered on Day 1, a second dose (C3D2) of etoposide to be administered on Day 2, and a third dose (C3D3) of etoposide to be administered on Day 3, wherein the C3D1, the C3D2, and the C3D3 of etoposide are each about 100 mg/m².

466. Use of glofitamab for treating a subject having a CD20-positive cell proliferative disorder, wherein glofitamab is to be administered in combination with obinutuzumab, rituximab, ifosfamide, carboplatin, and etoposide in a dosing regimen comprising a first dosing cycle, a second dosing cycle, and a third dosing cycle, wherein
(a) the first dosing cycle comprises:
   (i) a first dose (C1D1) of glofitamab to be administered on Day 8 and a second dose (C1D2) of glofitamab to be administered on Day 15, wherein the C1D1 of glofitamab is about 2.5 mg, and the C1D2 of glofitamab is about 10 mg;
   (ii) a first dose (C1D1) of obinutuzumab to be administered on Day 1, wherein the C1D1 of obinutuzumab is about 1000 mg;
   (iii) a single dose (C1D1) of ifosfamide to be administered on Day 2, wherein the C1D1 of ifosfamide is about 5000 mg/m² with a maximum dose of about 800 mg;
   (iv) a single dose (C1D1) of carboplatin to be administered on Day 2, wherein the C1D1 of carboplatin is about 5×(25+creatinine clearance) mg; and
   (v) a first dose (C1D1) of etoposide to be administered on Day 1, a second dose (C1D2) of etoposide to be administered on Day 2, and a third dose (C1D3) of etoposide to be administered on Day 3, wherein the C1D1, the C1D2, and the C1D3 of etoposide are each about 100 mg/m²;
(b) the second dosing cycle comprises:
   (i) a single dose (C2D1) of glofitamab to be administered on Day 8, wherein the C2D1 of glofitamab is about 30 mg;
   (ii) a first dose (C2D1) of rituximab to be administered on Day 1, wherein the C2D1 of rituximab is about 375 mg/m²;
   (iii) a single dose (C2D1) of ifosfamide to be administered on Day 2, wherein the C2D1 of ifosfamide is about 5000 mg/m² with a maximum dose of about 800 mg;
   (iv) a single dose (C2D1) of carboplatin to be administered on Day 2, wherein the C2D1 of carboplatin is about 5×(25+creatinine clearance) mg; and
   (v) a first dose (C2D1) of etoposide to be administered on Day 1, a second dose (C2D2) of etoposide to be administered on Day 2, and a third dose (C2D3) of etoposide to be administered on Day 3, wherein the C2D1, the C2D2, and the C2D3 of etoposide are each about 100 mg/m²; and
(c) the third dosing cycle comprises:
   (i) a single dose (C3D1) of glofitamab to be administered on Day 8, wherein the C3D1 of glofitamab is about 30 mg;
   (ii) a first dose (C3D1) of rituximab to be administered on Day 1, wherein the C3D1 of rituximab is about 375 mg/m²;
   (iii) a single dose (C3D1) of ifosfamide to be administered on Day 2, wherein the C3D1 of ifosfamide is about 5000 mg/m² with a maximum dose of about 800 mg;
   (iv) a single dose (C3D1) of carboplatin to be administered on Day 2, wherein the C3D1 of carboplatin is about 5×(25+creatinine clearance) mg; and
   (v) a first dose (C3D1) of etoposide to be administered on Day 1, a second dose (C3D2) of etoposide to be administered on Day 2, and a third dose (C3D3) of etoposide to be administered on Day 3, wherein the C3D1, the C3D2, and the C3D3 of etoposide are each about 100 mg/m².

467. The glofitamab for use or use of any one of embodiments 455 to 466, wherein mesna is to be administered simultaneously with any dose of ifosfamide.

468. The glofitamab for use or use of embodiment 467, wherein mesna is to be administered at a dose of about 5000 mg/m² intravenously.

469. The glofitamab for use or use of embodiment 468, wherein mesna is to be administered via continuous infusion over about 24 hours on Day 2 of each dosing cycle.

470. Glofitamab for use in a method of treating a subject aged between 6 months and 17 years having a CD20-positive cell proliferative disorder, wherein glofitamab is to be administered in combination with obinutuzumab, rituximab, ifosfamide, carboplatin, and etoposide in a dosing regimen comprising at least a first dosing cycle and a second dosing cycle, wherein
(a) the first dosing cycle comprises a first dose (C1D1) of glofitamab to be administered on Day 8 and a second dose (C1D2) of glofitamab to be administered on Day 15, wherein the C1D1 of glofitamab is about 0.03 mg/kg, about 0.04 mg/kg, or about 2.5 mg, and the C1D2 of glofitamab is about 0.15 mg/kg or about 10 mg; and
(b) the second dosing cycle comprises a single dose (C2D1) of glofitamab to be administered on Day 8, wherein the C2D1 of glofitamab is about 0.4 mg/kg, about 0.5 mg/kg, or about 30 mg.

471. Use of glofitamab in the manufacture of a medicament for the treatment of a subject aged between 6 months and 17 years having a CD20-positive cell proliferative disorder, wherein in said treatment glofitamab is to be administered in combination with obinutuzumab, rituximab, ifosfamide, carboplatin, and etoposide in a dosing regimen comprising at least a first dosing cycle and a second dosing cycle, wherein
(a) the first dosing cycle comprises a first dose (C1D1) of glofitamab to be administered on Day 8 and a second dose (C1D2) of glofitamab to be administered on Day 15, wherein the C1D1 of glofitamab is about 0.03 mg/kg, about 0.04 mg/kg, or about 2.5 mg, and the C1D2 of glofitamab is about 0.15 mg/kg or about 10 mg; and
(b) the second dosing cycle comprises a single dose (C2D1) of glofitamab to be administered on Day 8, wherein the C2D1 of glofitamab is about 0.4 mg/kg, about 0.5 mg/kg, or about 30 mg.

472. Use of glofitamab for treating a subject aged between 6 months and 17 years having a CD20-positive cell proliferative disorder, wherein glofitamab is to be administered in combination with obinutuzumab, rituximab, ifosfamide, carboplatin, and etoposide in a dosing regimen comprising at least a first dosing cycle and a second dosing cycle, wherein
(a) the first dosing cycle comprises a first dose (C1D1) of glofitamab to be administered on Day 8 and a second dose (C1D2) of glofitamab to be administered on Day 15, wherein the C1D1 of glofitamab is about 0.03 mg/kg, about 0.04 mg/kg, or about 2.5 mg, and the C1D2 of glofitamab is about 0.15 mg/kg or about 10 mg; and
(b) the second dosing cycle comprises a single dose (C2D1) of glofitamab to be administered on Day 8, wherein the C2D1 of glofitamab is about 0.4 mg/kg, about 0.5 mg/kg, or about 30 mg.

473. Glofitamab for use in a method of treating a subject aged between 6 months and 17 years having a CD20-positive cell proliferative disorder, wherein glofitamab is to be administered in combination with obinutuzumab, rituximab, ifosfamide, carboplatin, and etoposide in a dosing regimen comprising a first dosing cycle, a second dosing cycle, and a third dosing cycle, wherein
(a) the first dosing cycle comprises a first dose (C1D1) of glofitamab to be administered on Day 8 and a second dose (C1D2) of glofitamab to be administered on Day 15, wherein the C1D1 of glofitamab is about 0.03 mg/kg, about 0.04 mg/kg, or about 2.5 mg, and the C1D2 of glofitamab is about 0.15 mg/kg or about 10 mg;
(b) the second dosing cycle comprises a single dose (C2D1) of glofitamab to be administered on Day 8, wherein the C2D1 of glofitamab is about 0.4 mg/kg, about 0.5 mg/kg, or about 30 mg; and
(c) the third dosing cycle comprises a single dose (C3D1) of glofitamab to be administered on Day 8, wherein the C3D1 of glofitamab is about 0.4 mg/kg, about 0.5 mg/kg, or about 30 mg.

474. Use of glofitamab in the manufacture of a medicament for the treatment of a subject aged between 6 months and 17 years having a CD20-positive cell proliferative disorder, wherein in said treatment glofitamab is to be administered in combination with obinutuzumab, rituximab, ifosfamide, carboplatin, and etoposide in a dosing regimen comprising a first dosing cycle, a second dosing cycle, and a third dosing cycle, wherein
(a) the first dosing cycle comprises a first dose (C1D1) of glofitamab to be administered on Day 8 and a second dose (C1D2) of glofitamab to be administered on Day 15, wherein the C1D1 of glofitamab is about 0.03 mg/kg, about 0.04 mg/kg, or about 2.5 mg, and the C1D2 of glofitamab is about 0.15 mg/kg or about 10 mg;
(b) the second dosing cycle comprises a single dose (C2D1) of glofitamab to be administered on Day 8, wherein the C2D1 of glofitamab is about 0.4 mg/kg, about 0.5 mg/kg, or about 30 mg; and
(c) the third dosing cycle comprises a single dose (C3D1) of glofitamab to be administered on Day 8, wherein the C3D1 of glofitamab is about 0.4 mg/kg, about 0.5 mg/kg, or about 30 mg.

475. Use of glofitamab for treating a subject aged between 6 months and 17 years having a CD20-positive cell proliferative disorder, wherein glofitamab is to be administered in combination with obinutuzumab, rituximab, ifosfamide, carboplatin, and etoposide in a dosing regimen comprising a first dosing cycle, a second dosing cycle, and a third dosing cycle, wherein
(a) the first dosing cycle comprises a first dose (C1D1) of glofitamab to be administered on Day 8 and a second dose (C1D2) of glofitamab to be administered on Day 15, wherein the C1D1 of glofitamab is about 0.03 mg/kg, about 0.04 mg/kg, or about 2.5 mg, and the C1D2 of glofitamab is about 0.15 mg/kg or about 10 mg;
(b) the second dosing cycle comprises a single dose (C2D1) of glofitamab to be administered on Day 8, wherein the C2D1 of glofitamab is about 0.4 mg/kg, about 0.5 mg/kg, or about 30 mg; and
(c) the third dosing cycle comprises a single dose (C3D1) of glofitamab to be administered on Day 8, wherein the C3D1 of glofitamab is about 0.4 mg/kg, about 0.5 mg/kg, or about 30 mg.

476. Glofitamab for use in a method of treating a subject aged between 6 months and 17 years having a CD20-positive cell proliferative disorder, wherein glofitamab is to be administered in combination with obinutuzumab, rituximab, ifosfamide, carboplatin, and etoposide in a dosing regimen comprising at least a first dosing cycle and a second dosing cycle, wherein
(a) the first dosing cycle comprises:
   (i) a first dose (C1D1) of glofitamab to be administered on Day 8 and a second dose (C1D2) of glofitamab to be administered on Day 15, wherein the C1D1 of glofitamab is about 0.03 mg/kg, about 0.04 mg/kg, or about 2.5 mg, and the C1D2 of glofitamab is about 0.15 mg/kg or about 10 mg;
   (ii) a first dose (C1D1) of obinutuzumab to be administered on Day 1 and a second dose (C1D2) of obinutuzumab to be administered on Day 2, wherein the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab, and wherein the sum of the C1D1 and the C1D2 of obinutuzumab is about 38 mg/kg, about 28 mg/kg, about 23 mg/kg, about 20 mg/kg, or about 1000 mg;
   (iii) a first dose (C1D1) of ifosfamide to be administered on Day 3, a second dose (C1D2) of ifosfamide to be administered on Day 4, and a third dose (C1D3) of ifosfamide to be administered on Day 5, wherein the C1D1, the C1D2, and the C1D3 of ifosfamide are each about 3000 mg/m²;
   (iv) a single dose (C1D1) of carboplatin to be administered on Day 3, wherein the C1D1 of carboplatin is about 635 mg/m²; and
   (v) a first dose (C1D1) of etoposide to be administered on Day 3, a second dose (C1D2) of etoposide to be administered on Day 4, and a third dose (C1D3) of etoposide to be administered on Day 5, wherein the C1D1, the C1D2, and the C1D3 of etoposide are each about 100 mg/m²; and
(b) the second dosing cycle comprises:
   (i) a single dose (C2D1) of glofitamab to be administered on Day 1, wherein the C2D1 of glofitamab is about 0.4 mg/kg, about 0.5 mg/kg, or about 30 mg;
   (ii) a first dose (C2D1) of rituximab to be administered on Day 5, wherein the C2D1 of rituximab is about 375 mg/m²;
   (iii) a first dose (C2D1) of ifosfamide to be administered on Day 6, a second dose (C2D2) of ifosfamide to be administered on Day 7, and a third dose (C2D3) of ifosfamide to be administered on Day 8, wherein the C2D1, the C2D2, and the C2D3 of ifosfamide are each about 3000 mg/m²;
   (iv) a single dose (C2D1) of carboplatin to be administered on Day 6, wherein the C2D1 of carboplatin is about 5×(25+creatinine clearance) mg; and
   (v) a first dose (C2D1) of etoposide to be administered on Day 6, a second dose (C2D2) of etoposide to be administered on Day 7, and a third dose (C2D3) of etoposide to be administered on Day 8, wherein the C2D1, the C2D2, and the C2D3 of etoposide are each about 100 mg/m².

477. Use of glofitamab in the manufacture of a medicament for the treatment of a subject aged between 6 months and 17 years having a CD20-positive cell proliferative disorder, wherein in said treatment glofitamab is to be administered in combination with obinutuzumab, rituximab, ifosfamide, carboplatin, and etoposide in a dosing regimen comprising at least a first dosing cycle and a second dosing cycle, wherein
(a) the first dosing cycle comprises:
   (i) a first dose (C1D1) of glofitamab to be administered on Day 8 and a second dose (C1D2) of glofitamab to be administered on Day 15, wherein the C1D1 of glofitamab is about 0.03 mg/kg, about 0.04 mg/kg, or about 2.5 mg, and the C1D2 of glofitamab is about 0.15 mg/kg or about 10 mg;
   (ii) a first dose (C1D1) of obinutuzumab to be administered on Day 1 and a second dose (C1D2) of obinutuzumab to be administered on Day 2, wherein the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab, and wherein the sum of the C1D1 and the C1D2 of obinutuzumab is about 38 mg/kg, about 28 mg/kg, about 23 mg/kg, about 20 mg/kg, or about 1000 mg;
   (iii) a first dose (C1D1) of ifosfamide to be administered on Day 3, a second dose (C1D2) of ifosfamide to be administered on Day 4, and a third dose (C1D3) of ifosfamide to be administered on Day 5, wherein the C1D1, the C1D2, and the C1D3 of ifosfamide are each about 3000 mg/m²;
   (iv) a single dose (C1D1) of carboplatin to be administered on Day 3, wherein the C1D1 of carboplatin is about 635 mg/m²; and
   (v) a first dose (C1D1) of etoposide to be administered on Day 3, a second dose (C1D2) of etoposide to be administered on Day 4, and a third dose (C1D3) of etoposide to be administered on Day 5, wherein the C1D1, the C1D2, and the C1D3 of etoposide are each about 100 mg/m²; and
(b) the second dosing cycle comprises:
   (i) a single dose (C2D1) of glofitamab to be administered on Day 1, wherein the C2D1 of glofitamab is about 0.4 mg/kg, about 0.5 mg/kg, or about 30 mg;
   (ii) a first dose (C2D1) of rituximab to be administered on Day 5, wherein the C2D1 of rituximab is about 375 mg/m²;
   (iii) a first dose (C2D1) of ifosfamide to be administered on Day 6, a second dose (C2D2) of ifosfamide to be administered on Day 7, and a third dose (C2D3) of ifosfamide to be administered on Day 8, wherein the C2D1, the C2D2, and the C2D3 of ifosfamide are each about 3000 mg/m²;
   (iv) a single dose (C2D1) of carboplatin to be administered on Day 6, wherein the C2D1 of carboplatin is about 5×(25+creatinine clearance) mg; and
   (v) a first dose (C2D1) of etoposide to be administered on Day 6, a second dose (C2D2) of etoposide to be administered on Day 7, and a third dose (C2D3) of etoposide to be administered on Day 8, wherein the C2D1, the C2D2, and the C2D3 of etoposide are each about 100 mg/m².

478. Use of glofitamab for treating a subject aged between 6 months and 17 years having a CD20-positive cell proliferative disorder, wherein glofitamab is to be administered in combination with obinutuzumab, rituximab, ifosfamide, carboplatin, and etoposide in a dosing regimen comprising at least a first dosing cycle and a second dosing cycle, wherein
(a) the first dosing cycle comprises:
   (i) a first dose (C1D1) of glofitamab to be administered on Day 8 and a second dose (C1D2) of glofitamab to be administered on Day 15, wherein the C1D1 of glofitamab is about 0.03 mg/kg, about 0.04 mg/kg, or about 2.5 mg, and the C1D2 of glofitamab is about 0.15 mg/kg or about 10 mg;
   (ii) a first dose (C1D1) of obinutuzumab to be administered on Day 1 and a second dose (C1D2) of obinutuzumab to be administered on Day 2, wherein the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab, and wherein the sum of the C1D1 and the C1D2 of obinutuzumab is about 38 mg/kg, about 28 mg/kg, about 23 mg/kg, about 20 mg/kg, or about 1000 mg;
   (iii) a first dose (C1D1) of ifosfamide to be administered on Day 3, a second dose (C1D2) of ifosfamide to be administered on Day 4, and a third dose (C1D3) of ifosfamide to be administered on Day 5, wherein the C1D1, the C1D2, and the C1D3 of ifosfamide are each about 3000 mg/m²;
   (iv) a single dose (C1D1) of carboplatin to be administered on Day 3, wherein the C1D1 of carboplatin is about 635 mg/m²; and
   (v) a first dose (C1D1) of etoposide to be administered on Day 3, a second dose (C1D2) of etoposide to be administered on Day 4, and a third dose (C1D3) of etoposide to be administered on Day 5, wherein the C1D1, the C1D2, and the C1D3 of etoposide are each about 100 mg/m²; and
(b) the second dosing cycle comprises:
   (i) a single dose (C2D1) of glofitamab to be administered on Day 1, wherein the C2D1 of glofitamab is about 0.4 mg/kg, about 0.5 mg/kg, or about 30 mg;
   (ii) a first dose (C2D1) of rituximab to be administered on Day 5, wherein the C2D1 of rituximab is about 375 mg/m²;
   (iii) a first dose (C2D1) of ifosfamide to be administered on Day 6, a second dose (C2D2) of ifosfamide to be administered on Day 7, and a third dose (C2D3) of ifosfamide to be administered on Day 8, wherein the C2D1, the C2D2, and the C2D3 of ifosfamide are each about 3000 mg/m²;
   (iv) a single dose (C2D1) of carboplatin to be administered on Day 6, wherein the C2D1 of carboplatin is about 5×(25+creatinine clearance) mg; and
   (v) a first dose (C2D1) of etoposide to be administered on Day 6, a second dose (C2D2) of etoposide to be administered on Day 7, and a third dose (C2D3) of etoposide to be administered on Day 8, wherein the C2D1, the C2D2, and the C2D3 of etoposide are each about 100 mg/m².

479. Glofitamab for use in a method of treating a subject aged between 6 months and 17 years having a CD20-positive cell proliferative disorder, wherein glofitamab is to be administered in combination with obinutuzumab, rituximab, ifosfamide, carboplatin, and etoposide in a dosing regimen comprising a first dosing cycle, a second dosing cycle, and a third dosing cycle, wherein
(a) the first dosing cycle comprises:
   (i) a first dose (C1D1) of glofitamab to be administered on Day 8 and a second dose (C1D2) of glofitamab to be administered on Day 15, wherein the C1D1 of glofitamab is about 0.03 mg/kg, about 0.04 mg/kg, or about 2.5 mg, and the C1D2 of glofitamab is about 0.15 mg/kg or about 10 mg;
   (ii) a first dose (C1D1) of obinutuzumab to be administered on Day 1 and a second dose (C1D2) of obinutuzumab to be administered on Day 2, wherein the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab, and wherein the sum of the C1D1 and the C1D2 of obinutuzumab is about 38 mg/kg, about 28 mg/kg, about 23 mg/kg, about 20/kg, or about 1000 mg;
   (iii) a first dose (C1D1) of ifosfamide to be administered on Day 3, a second dose (C1D2) of ifosfamide to be administered on Day 4, and a third dose (C1D3) of ifosfamide to be administered on Day 5, wherein the C1D1, the C1D2, and the C1D3 of ifosfamide are each about 3000 mg/m²;
   (iv) a single dose (C1D1) of carboplatin to be administered on Day 3, wherein the C1D1 of carboplatin is about 635 mg/m²; and
   (v) a first dose (C1D1) of etoposide to be administered on Day 3, a second dose (C1D2) of etoposide to be administered on Day 4, and a third dose (C1D3) of etoposide to be administered on Day 5, wherein the C1D1, the C1D2, and the C1D3 of etoposide are each about 100 mg/m²;
(b) the second dosing cycle comprises:
   (i) a single dose (C2D1) of glofitamab to be administered on Day 1, wherein the C2D1 of glofitamab is about 0.4 mg/kg, about 0.5 mg/kg, or about 30 mg;
   (ii) a first dose (C2D1) of rituximab to be administered on Day 5, wherein the C2D1 of rituximab is about 375 mg/m²;
   (iii) a first dose (C2D1) of ifosfamide to be administered on Day 6, a second dose (C2D2) of ifosfamide to be administered on Day 7, and a third dose (C2D3) of ifosfamide to be administered on Day 8, wherein the C2D1, the C2D2, and the C2D3 of ifosfamide are each about 3000 mg/m²;
   (iv) a single dose (C2D1) of carboplatin to be administered on Day 6, wherein the C2D1 of carboplatin is about 5×(25+creatinine clearance) mg; and
   (v) a first dose (C2D1) of etoposide to be administered on Day 6, a second dose (C2D2) of etoposide to be administered on Day 7, and a third dose (C2D3) of etoposide to be administered on Day 8, wherein the C2D1, the C2D2, and the C2D3 of etoposide are each about 100 mg/m²; and
(c) the third dosing cycle comprises:
   (i) a single dose (C3D1) of glofitamab to be administered on Day 1, wherein the C3D1 of glofitamab is about 0.4 mg/kg, about 0.5 mg/kg, or about 30 mg;
   (ii) a first dose (C3D1) of rituximab to be administered on Day 5, wherein the C3D1 of rituximab is about 375 mg/m²;
   (iii) a first dose (C3D1) of ifosfamide to be administered on Day 6, a second dose (C3D2) of ifosfamide to be administered on Day 7, and a third dose (C3D3) of ifosfamide to be administered on Day 8, wherein the C3D1, the C3D2, and the C3D3 of ifosfamide are each about 3000 mg/m²;
   (iv) a single dose (C3D1) of carboplatin to be administered on Day 6, wherein the C3D1 of carboplatin is about 5×(25+creatinine clearance) mg; and
   (v) a first dose (C3D1) of etoposide to be administered on Day 6, a second dose (C3D2) of etoposide to be administered on Day 7, and a third dose (C3D3) of etoposide to be administered on Day 8, wherein the C3D1, the C3D2, and the C3D3 of etoposide are each about 100 mg/m².

480. Use of glofitamab in the manufacture of a medicament for the treatment of a subject aged between 6 months and 17 years having a CD20-positive cell proliferative disorder, wherein in said treatment glofitamab is to be administered in combination with obinutuzumab, rituximab, ifosfamide, carboplatin, and etoposide in a dosing regimen comprising a first dosing cycle, a second dosing cycle, and a third dosing cycle, wherein
(a) the first dosing cycle comprises:
   (i) a first dose (C1D1) of glofitamab to be administered on Day 8 and a second dose (C1D2) of glofitamab to be administered on Day 15, wherein the C1D1 of glofitamab is about 0.03 mg/kg, about 0.04 mg/kg, or about 2.5 mg, and the C1D2 of glofitamab is about 0.15 mg/kg or about 10 mg;
   (ii) a first dose (C1D1) of obinutuzumab to be administered on Day 1 and a second dose (C1D2) of obinutuzumab to be administered on Day 2, wherein the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab, and wherein the sum of the C1D1 and the C1D2 of obinutuzumab is about 38 mg/kg, about 28 mg/kg, about 23 mg/kg, about 20/kg, or about 1000 mg;
   (iii) a first dose (C1D1) of ifosfamide to be administered on Day 3, a second dose (C1D2) of ifosfamide to be administered on Day 4, and a third dose (C1D3) of ifosfamide to be administered on Day 5, wherein the C1D1, the C1D2, and the C1D3 of ifosfamide are each about 3000 mg/m²;
   (iv) a single dose (C1D1) of carboplatin to be administered on Day 3, wherein the C1D1 of carboplatin is about 635 mg/m²; and
   (v) a first dose (C1D1) of etoposide to be administered on Day 3, a second dose (C1D2) of etoposide to be administered on Day 4, and a third dose (C1D3) of etoposide to be administered on Day 5, wherein the C1D1, the C1D2, and the C1D3 of etoposide are each about 100 mg/m²;
(b) the second dosing cycle comprises:
   (i) a single dose (C2D1) of glofitamab to be administered on Day 1, wherein the C2D1 of glofitamab is about 0.4 mg/kg, about 0.5 mg/kg, or about 30 mg;
   (ii) a first dose (C2D1) of rituximab to be administered on Day 5, wherein the C2D1 of rituximab is about 375 mg/m²;
   (iii) a first dose (C2D1) of ifosfamide to be administered on Day 6, a second dose (C2D2) of ifosfamide to be administered on Day 7, and a third dose (C2D3) of ifosfamide to be administered on Day 8, wherein the C2D1, the C2D2, and the C2D3 of ifosfamide are each about 3000 mg/m²;
   (iv) a single dose (C2D1) of carboplatin to be administered on Day 6, wherein the C2D1 of carboplatin is about 5×(25+creatinine clearance) mg; and
   (v) a first dose (C2D1) of etoposide to be administered on Day 6, a second dose (C2D2) of etoposide to be administered on Day 7, and a third dose (C2D3) of etoposide to be administered on Day 8, wherein the C2D1, the C2D2, and the C2D3 of etoposide are each about 100 mg/m²; and
(c) the third dosing cycle comprises:
   (i) a single dose (C3D1) of glofitamab to be administered on Day 1, wherein the C3D1 of glofitamab is about 0.4 mg/kg, about 0.5 mg/kg, or about 30 mg;
   (ii) a first dose (C3D1) of rituximab to be administered on Day 5, wherein the C3D1 of rituximab is about 375 mg/m²;
   (iii) a first dose (C3D1) of ifosfamide to be administered on Day 6, a second dose (C3D2) of ifosfamide to be administered on Day 7, and a third dose (C3D3) of ifosfamide to be administered on Day 8, wherein the C3D1, the C3D2, and the C3D3 of ifosfamide are each about 3000 mg/m²;
   (iv) a single dose (C3D1) of carboplatin to be administered on Day 6, wherein the C3D1 of carboplatin is about 5×(25+creatinine clearance) mg; and
   (v) a first dose (C3D1) of etoposide to be administered on Day 6, a second dose (C3D2) of etoposide to be administered on Day 7, and a third dose (C3D3) of etoposide to be administered on Day 8, wherein the C3D1, the C3D2, and the C3D3 of etoposide are each about 100 mg/m².

481. Use of glofitamab for treating a subject aged between 6 months and 17 years having a CD20-positive cell proliferative disorder, wherein glofitamab is to be administered in combination with obinutuzumab, rituximab, ifosfamide, carboplatin, and etoposide in a dosing regimen comprising a first dosing cycle, a second dosing cycle, and a third dosing cycle, wherein
(a) the first dosing cycle comprises:
   (i) a first dose (C1D1) of glofitamab to be administered on Day 8 and a second dose (C1D2) of glofitamab to be administered on Day 15, wherein the C1D1 of glofitamab is about 0.03 mg/kg, about 0.04 mg/kg, or about 2.5 mg, and the C1D2 of glofitamab is about 0.15 mg/kg or about 10 mg;
   (ii) a first dose (C1D1) of obinutuzumab to be administered on Day 1 and a second dose (C1D2) of obinutuzumab to be administered on Day 2, wherein the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab, and wherein the sum of the C1D1 and the C1D2 of obinutuzumab is about 38 mg/kg, about 28 mg/kg, about 23 mg/kg, about 20/kg, or about 1000 mg;
   (iii) a first dose (C1D1) of ifosfamide to be administered on Day 3, a second dose (C1D2) of ifosfamide to be administered on Day 4, and a third dose (C1D3) of ifosfamide to be administered on Day 5, wherein the C1D1, the C1D2, and the C1D3 of ifosfamide are each about 3000 mg/m²;
   (iv) a single dose (C1D1) of carboplatin to be administered on Day 3, wherein the C1D1 of carboplatin is about 635 mg/m²; and
   (v) a first dose (C1D1) of etoposide to be administered on Day 3, a second dose (C1D2) of etoposide to be administered on Day 4, and a third dose (C1D3) of etoposide to be administered on Day 5, wherein the C1D1, the C1D2, and the C1D3 of etoposide are each about 100 mg/m²;
(b) the second dosing cycle comprises:
   (i) a single dose (C2D1) of glofitamab to be administered on Day 1, wherein the C2D1 of glofitamab is about 0.4 mg/kg, about 0.5 mg/kg, or about 30 mg;
   (ii) a first dose (C2D1) of rituximab to be administered on Day 5, wherein the C2D1 of rituximab is about 375 mg/m²;
   (iii) a first dose (C2D1) of ifosfamide to be administered on Day 6, a second dose (C2D2) of ifosfamide to be administered on Day 7, and a third dose (C2D3) of ifosfamide to be administered on Day 8, wherein the C2D1, the C2D2, and the C2D3 of ifosfamide are each about 3000 mg/m²;
   (iv) a single dose (C2D1) of carboplatin to be administered on Day 6, wherein the C2D1 of carboplatin is about 5×(25+creatinine clearance) mg; and
   (v) a first dose (C2D1) of etoposide to be administered on Day 6, a second dose (C2D2) of etoposide to be administered on Day 7, and a third dose (C2D3) of etoposide to be administered on Day 8, wherein the C2D1, the C2D2, and the C2D3 of etoposide are each about 100 mg/m²; and
(c) the third dosing cycle comprises:
   (i) a single dose (C3D1) of glofitamab to be administered on Day 1, wherein the C3D1 of glofitamab is about 0.4 mg/kg, about 0.5 mg/kg, or about 30 mg;
   (ii) a first dose (C3D1) of rituximab to be administered on Day 5, wherein the C3D1 of rituximab is about 375 mg/m²;
   (iii) a first dose (C3D1) of ifosfamide to be administered on Day 6, a second dose (C3D2) of ifosfamide to be administered on Day 7, and a third dose (C3D3) of ifosfamide to be administered on Day 8, wherein the C3D1, the C3D2, and the C3D3 of ifosfamide are each about 3000 mg/m²;
   (iv) a single dose (C3D1) of carboplatin to be administered on Day 6, wherein the C3D1 of carboplatin is about 5×(25+creatinine clearance) mg; and
   (v) a first dose (C3D1) of etoposide to be administered on Day 6, a second dose (C3D2) of etoposide to be administered on Day 7, and a third dose (C3D3) of etoposide to be administered on Day 8, wherein the C3D1, the C3D2, and the C3D3 of etoposide are each about 100 mg/m².

482. The glofitamab for use or use of any one of embodiments 470 to 481, wherein mesna is to be administered daily to the subject on Days 3, 4, and 5 of the first dosing cycle, on Days 6, 7, and 8 of the second dosing cycle, and/or on Days 6, 7, and 8 of each additional dosing cycle.

483. The glofitamab for use or use of embodiment 482, wherein mesna is to be administered intravenously daily as five doses totaling 3000 mg/m² in amount.

484. The glofitamab for use or use of embodiment 483, wherein mesna is to be administered intravenously at a first dose of about 600 mg/m² prior to the administration of any dose of ifosfamide and at four repeated doses of about 600 mg/m² each at about three hours, about six hours, about nine hours, and about 12 hours, respectively, after the first dose of ifosfamide.

485. Glofitamab for use in a method of treating a subject aged between 18 years and 30 years having a CD20-positive cell proliferative disorder, wherein glofitamab is to be administered in combination with obinutuzumab, rituximab, ifosfamide, carboplatin, and etoposide in a dosing regimen comprising at least a first dosing cycle and a second dosing cycle, wherein
(a) the first dosing cycle comprises a first dose (C1D1) of glofitamab to be administered on Day 8 and a second dose (C1D2) of glofitamab to be administered on Day 15, wherein the C1D1 of glofitamab is about 2.5 mg, and the C1D2 of glofitamab is about 10 mg; and
(b) the second dosing cycle comprises a single dose (C2D1) of glofitamab to be administered on Day 1, wherein the C2D1 of glofitamab is about 30 mg.

486. Use of glofitamab in the manufacture of a medicament for the treatment of a subject aged between 18 years and 30 years having a CD20-positive cell proliferative disorder, wherein in said treatment glofitamab is to be administered in combination with obinutuzumab, rituximab, ifosfamide, carboplatin, and etoposide in a dosing regimen comprising at least a first dosing cycle and a second dosing cycle, wherein
(a) the first dosing cycle comprises a first dose (C1D1) of glofitamab to be administered on Day 8 and a second dose (C1D2) of glofitamab to be administered on Day 15, wherein the C1D1 of glofitamab is about 2.5 mg, and the C1D2 of glofitamab is about 10 mg; and
(b) the second dosing cycle comprises a single dose (C2D1) of glofitamab to be administered on Day 1, wherein the C2D1 of glofitamab is about 30 mg.

487. Use of glofitamab for treating a subject aged between 18 years and 30 years having a CD20-positive cell proliferative disorder, wherein glofitamab is to be administered in combination with obinutuzumab, rituximab, ifosfamide, carboplatin, and etoposide in a dosing regimen comprising at least a first dosing cycle and a second dosing cycle, wherein
(a) the first dosing cycle comprises a first dose (C1D1) of glofitamab to be administered on Day 8 and a second dose (C1D2) of glofitamab to be administered on Day 15, wherein the C1D1 of glofitamab is about 2.5 mg, and the C1D2 of glofitamab is about 10 mg; and
(b) the second dosing cycle comprises a single dose (C2D1) of glofitamab to be administered on Day 1, wherein the C2D1 of glofitamab is about 30 mg.

488. Glofitamab for use in a method of treating a subject aged between 18 years and 30 years having a CD20-positive cell proliferative disorder, wherein glofitamab is to be administered in combination with obinutuzumab, rituximab, ifosfamide, carboplatin, and etoposide in a dosing regimen comprising a first dosing cycle, a second dosing cycle, and a third dosing cycle, wherein
(a) the first dosing cycle comprises a first dose (C1D1) of glofitamab to be administered on Day 8 and a second dose (C1D2) of glofitamab to be administered on Day 15, wherein the C1D1 of glofitamab is about 2.5 mg, and the C1D2 of glofitamab is about 10 mg;
(b) the second dosing cycle comprises a single dose (C2D1) of glofitamab to be administered on Day 1, wherein the C2D1 of glofitamab is about 30 mg; and
(c) the third dosing cycle comprises a single dose (C3D1) of glofitamab to be administered on Day 1, wherein the C3D1 of glofitamab is about 30 mg.

489. Use of glofitamab in the manufacture of a medicament for the treatment of a subject aged between 18 years and 30 years having a CD20-positive cell proliferative disorder, wherein in said treatment glofitamab is to be administered in combination with obinutuzumab, rituximab, ifosfamide, carboplatin, and etoposide in a dosing regimen comprising a first dosing cycle, a second dosing cycle, and a third dosing cycle, wherein
(a) the first dosing cycle comprises a first dose (C1D1) of glofitamab to be administered on Day 8 and a second dose (C1D2) of glofitamab to be administered on Day 15, wherein the C1D1 of glofitamab is about 2.5 mg, and the C1D2 of glofitamab is about 10 mg;
(b) the second dosing cycle comprises a single dose (C2D1) of glofitamab to be administered on Day 1, wherein the C2D1 of glofitamab is about 30 mg; and
(c) the third dosing cycle comprises a single dose (C3D1) of glofitamab to be administered on Day 1, wherein the C3D1 of glofitamab is about 30 mg.

490. Use of glofitamab for treating a subject aged between 18 years and 30 years having a CD20-positive cell proliferative disorder, wherein glofitamab is to be administered in combination with obinutuzumab, rituximab, ifosfamide, carboplatin, and etoposide in a dosing regimen comprising a first dosing cycle, a second dosing cycle, and a third dosing cycle, wherein
(a) the first dosing cycle comprises a first dose (C1D1) of glofitamab to be administered on Day 8 and a second dose (C1D2) of glofitamab to be administered on Day 15, wherein the C1D1 of glofitamab is about 2.5 mg, and the C1D2 of glofitamab is about 10 mg;
(b) the second dosing cycle comprises a single dose (C2D1) of glofitamab to be administered on Day 1, wherein the C2D1 of glofitamab is about 30 mg; and
(c) the third dosing cycle comprises a single dose (C3D1) of glofitamab to be administered on Day 1, wherein the C3D1 of glofitamab is about 30 mg.

491. Glofitamab for use in a method of treating a subject aged between 18 years and 30 years having a CD20-positive cell proliferative disorder, wherein glofitamab is to be administered in combination with obinutuzumab, rituximab, ifosfamide, carboplatin, and etoposide in a dosing regimen comprising at least a first dosing cycle and a second dosing cycle, wherein
(a) the first dosing cycle comprises:
   (i) a first dose (C1D1) of glofitamab to be administered on Day 8 and a second dose (C1D2) of glofitamab to be administered on Day 15, wherein the C1D1 of glofitamab is about 2.5 mg, and the C1D2 of glofitamab is about 10 mg;
   (ii) a first dose (C1D1) of obinutuzumab to be administered on Day 1 and a second dose (C1D2) of obinutuzumab to be administered on Day 2, wherein the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab, and wherein the sum of the C1D1 and the C1D2 of obinutuzumab is about 1000 mg;
   (iii) a single dose (C1D1) of ifosfamide to be administered on Day 3, wherein the C1D1 of ifosfamide is about 5000 mg/m² with a maximum dose of about 800 mg;
   (iv) a single dose (C1D1) of carboplatin to be administered on Day 3, wherein the C1D1 of carboplatin is about 5×(25+creatinine clearance) mg; and
   (v) a first dose (C1D1) of etoposide to be administered on Day 3, a second dose (C1D2) of etoposide to be administered on Day 4, and a third dose (C1D3) of etoposide to be administered on Day 5, wherein the C1D1, the C1D2, and the C1D3 of etoposide are each about 100 mg/m²; and
(b) the second dosing cycle comprises:
   (i) a single dose (C2D1) of glofitamab to be administered on Day 1, wherein the C2D1 of glofitamab is about 30 mg;
   (ii) a first dose (C2D1) of rituximab to be administered on Day 5, wherein the C2D1 of rituximab is about 375 mg/m²;
   (iii) a single dose (C2D1) of ifosfamide to be administered on Day 6, wherein the C2D1 of ifosfamide is about 5000 mg/m² with a maximum dose of about 800 mg;
   (iv) a single dose (C2D1) of carboplatin to be administered on Day 6, wherein the C2D1 of carboplatin is about 5×(25+creatinine clearance) mg; and
   (v) a first dose (C2D1) of etoposide to be administered on Day 6, a second dose (C2D2) of etoposide to be administered on Day 7, and a third dose (C2D3) of etoposide to be administered on Day 8, wherein the C2D1, the C2D2, and the C2D3 of etoposide are each about 100 mg/m².

492. Use of glofitamab in the manufacture of a medicament for the treatment of a subject aged between 18 years and 30 years having a CD20-positive cell proliferative disorder, wherein in said treatment glofitamab is to be administered in combination with obinutuzumab, rituximab, ifosfamide, carboplatin, and etoposide in a dosing regimen comprising at least a first dosing cycle and a second dosing cycle, wherein
(a) the first dosing cycle comprises:
   (i) a first dose (C1D1) of glofitamab to be administered on Day 8 and a second dose (C1D2) of glofitamab to be administered on Day 15, wherein the C1D1 of glofitamab is about 2.5 mg, and the C1D2 of glofitamab is about 10 mg;
   (ii) a first dose (C1D1) of obinutuzumab to be administered on Day 1 and a second dose (C1D2) of obinutuzumab to be administered on Day 2, wherein the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab, and wherein the sum of the C1D1 and the C1D2 of obinutuzumab is about 1000 mg;
   (iii) a single dose (C1D1) of ifosfamide to be administered on Day 3, wherein the C1D1 of ifosfamide is about 5000 mg/m² with a maximum dose of about 800 mg;
   (iv) a single dose (C1D1) of carboplatin to be administered on Day 3, wherein the C1D1 of carboplatin is about 5×(25+creatinine clearance) mg; and
   (v) a first dose (C1D1) of etoposide to be administered on Day 3, a second dose (C1D2) of etoposide to be administered on Day 4, and a third dose (C1D3) of etoposide to be administered on Day 5, wherein the C1D1, the C1D2, and the C1D3 of etoposide are each about 100 mg/m²; and
(b) the second dosing cycle comprises:
   (i) a single dose (C2D1) of glofitamab to be administered on Day 1, wherein the C2D1 of glofitamab is about 30 mg;
   (ii) a first dose (C2D1) of rituximab to be administered on Day 5, wherein the C2D1 of rituximab is about 375 mg/m²;
   (iii) a single dose (C2D1) of ifosfamide to be administered on Day 6, wherein the C2D1 of ifosfamide is about 5000 mg/m² with a maximum dose of about 800 mg;
   (iv) a single dose (C2D1) of carboplatin to be administered on Day 6, wherein the C2D1 of carboplatin is about 5×(25+creatinine clearance) mg; and
   (v) a first dose (C2D1) of etoposide to be administered on Day 6, a second dose (C2D2) of etoposide to be administered on Day 7, and a third dose (C2D3) of etoposide to be administered on Day 8, wherein the C2D1, the C2D2, and the C2D3 of etoposide are each about 100 mg/m².

493. Use of glofitamab for treating a subject aged between 18 years and 30 years having a CD20-positive cell proliferative disorder, wherein glofitamab is to be administered in combination with obinutuzumab, rituximab, ifosfamide, carboplatin, and etoposide in a dosing regimen comprising at least a first dosing cycle and a second dosing cycle, wherein
(a) the first dosing cycle comprises:
   (i) a first dose (C1D1) of glofitamab to be administered on Day 8 and a second dose (C1D2) of glofitamab to be administered on Day 15, wherein the C1D1 of glofitamab is about 2.5 mg, and the C1D2 of glofitamab is about 10 mg;
   (ii) a first dose (C1D1) of obinutuzumab to be administered on Day 1 and a second dose (C1D2) of obinutuzumab to be administered on Day 2, wherein the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab, and wherein the sum of the C1D1 and the C1D2 of obinutuzumab is about 1000 mg;
   (iii) a single dose (C1D1) of ifosfamide to be administered on Day 3, wherein the C1D1 of ifosfamide is about 5000 mg/m² with a maximum dose of about 800 mg;
   (iv) a single dose (C1D1) of carboplatin to be administered on Day 3, wherein the C1D1 of carboplatin is about 5×(25+creatinine clearance) mg; and
   (v) a first dose (C1D1) of etoposide to be administered on Day 3, a second dose (C1D2) of etoposide to be administered on Day 4, and a third dose (C1D3) of etoposide to be administered on Day 5, wherein the C1D1, the C1D2, and the C1D3 of etoposide are each about 100 mg/m²; and
(b) the second dosing cycle comprises:
   (i) a single dose (C2D1) of glofitamab to be administered on Day 1, wherein the C2D1 of glofitamab is about 30 mg;
   (ii) a first dose (C2D1) of rituximab to be administered on Day 5, wherein the C2D1 of rituximab is about 375 mg/m²;
   (iii) a single dose (C2D1) of ifosfamide to be administered on Day 6, wherein the C2D1 of ifosfamide is about 5000 mg/m² with a maximum dose of about 800 mg;
   (iv) a single dose (C2D1) of carboplatin to be administered on Day 6, wherein the C2D1 of carboplatin is about 5×(25+creatinine clearance) mg; and
   (v) a first dose (C2D1) of etoposide to be administered on Day 6, a second dose (C2D2) of etoposide to be administered on Day 7, and a third dose (C2D3) of etoposide to be administered on Day 8, wherein the C2D1, the C2D2, and the C2D3 of etoposide are each about 100 mg/m².

494. Glofitamab for use in a method of treating a subject aged between 18 years and 30 years having a CD20-positive cell proliferative disorder, wherein glofitamab is to be administered in combination with obinutuzumab, rituximab, ifosfamide, carboplatin, and etoposide in a dosing regimen comprising a first dosing cycle, a second dosing cycle, and a third dosing cycle, wherein
(a) the first dosing cycle comprises:
   (i) a first dose (C1D1) of glofitamab to be administered on Day 8 and a second dose (C1D2) of glofitamab to be administered on Day 15, wherein the C1D1 of glofitamab is about 2.5 mg, and the C1D2 of glofitamab is about 10 mg;
   (ii) a first dose (C1D1) of obinutuzumab to be administered on Day 1 and a second dose (C1D2) of obinutuzumab to be administered on Day 2, wherein the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab, and wherein the sum of the C1D1 and the C1D2 of obinutuzumab is about 1000 mg;
   (iii) a single dose (C1D1) of ifosfamide to be administered on Day 3, wherein the C1D1 of ifosfamide is about 5000 mg/m² with a maximum dose of about 800 mg;
   (iv) a single dose (C1D1) of carboplatin to be administered on Day 3, wherein the C1D1 of carboplatin is about 5×(25+creatinine clearance) mg; and
   (v) a first dose (C1D1) of etoposide to be administered on Day 3, a second dose (C1D2) of etoposide to be administered on Day 4, and a third dose (C1D3) of etoposide to be administered on Day 5, wherein the C1D1, the C1D2, and the C1D3 of etoposide are each about 100 mg/m²;
(b) the second dosing cycle comprises:
   (i) a single dose (C2D1) of glofitamab to be administered on Day 1, wherein the C2D1 of glofitamab is about 30 mg;
   (ii) a first dose (C2D1) of rituximab to be administered on Day 5, wherein the C2D1 of rituximab is about 375 mg/m²;
   (iii) a single dose (C2D1) of ifosfamide to be administered on Day 6, wherein the C2D1 of ifosfamide is about 5000 mg/m² with a maximum dose of about 800 mg;
   (iv) a single dose (C2D1) of carboplatin to be administered on Day 6, wherein the C2D1 of carboplatin is about 5×(25+creatinine clearance) mg; and
   (v) a first dose (C2D1) of etoposide to be administered on Day 6, a second dose (C2D2) of etoposide to be administered on Day 7, and a third dose (C2D3) of etoposide to be administered on Day 8, wherein the C2D1, the C2D2, and the C2D3 of etoposide are each about 100 mg/m²; and
(c) the third dosing cycle comprises:
   (i) a single dose (C3D1) of glofitamab to be administered on Day 1, wherein the C3D1 of glofitamab is about 30 mg;
   (ii) a first dose (C3D1) of rituximab to be administered on Day 5, wherein the C3D1 of rituximab is about 375 mg/m²;
   (iii) a single dose (C3D1) of ifosfamide to be administered on Day 6, wherein the C3D1 of ifosfamide is about 5000 mg/m² with a maximum dose of about 800 mg;
   (iv) a single dose (C3D1) of carboplatin to be administered on Day 6, wherein the C3D1 of carboplatin is about 5×(25+creatinine clearance) mg; and
   (v) a first dose (C3D1) of etoposide to be administered on Day 6, a second dose (C3D2) of etoposide to be administered on Day 7, and a third dose (C3D3) of etoposide to be administered on Day 8, wherein the C3D1, the C3D2, and the C3D3 of etoposide are each about 100 mg/m².

495. Use of glofitamab in the manufacture of a medicament for the treatment of a subject aged between 18 years and 30 years having a CD20-positive cell proliferative disorder, wherein in said treatment glofitamab is to be administered in combination with obinutuzumab, rituximab, ifosfamide, carboplatin, and etoposide in a dosing regimen comprising a first dosing cycle, a second dosing cycle, and a third dosing cycle, wherein
(a) the first dosing cycle comprises:
   (i) a first dose (C1D1) of glofitamab to be administered on Day 8 and a second dose (C1D2) of glofitamab to be administered on Day 15, wherein the C1D1 of glofitamab is about 2.5 mg, and the C1D2 of glofitamab is about 10 mg;
   (ii) a first dose (C1D1) of obinutuzumab to be administered on Day 1 and a second dose (C1D2) of obinutuzumab to be administered on Day 2, wherein the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab, and wherein the sum of the C1D1 and the C1D2 of obinutuzumab is about 1000 mg;
   (iii) a single dose (C1D1) of ifosfamide to be administered on Day 3, wherein the C1D1 of ifosfamide is about 5000 mg/m² with a maximum dose of about 800 mg;
   (iv) a single dose (C1D1) of carboplatin to be administered on Day 3, wherein the C1D1 of carboplatin is about 5×(25+creatinine clearance) mg; and
   (v) a first dose (C1D1) of etoposide to be administered on Day 3, a second dose (C1D2) of etoposide to be administered on Day 4, and a third dose (C1D3) of etoposide to be administered on Day 5, wherein the C1D1, the C1D2, and the C1D3 of etoposide are each about 100 mg/m²;
(b) the second dosing cycle comprises:
   (i) a single dose (C2D1) of glofitamab to be administered on Day 1, wherein the C2D1 of glofitamab is about 30 mg;
   (ii) a first dose (C2D1) of rituximab to be administered on Day 5, wherein the C2D1 of rituximab is about 375 mg/m²;
   (iii) a single dose (C2D1) of ifosfamide to be administered on Day 6, wherein the C2D1 of ifosfamide is about 5000 mg/m² with a maximum dose of about 800 mg;
   (iv) a single dose (C2D1) of carboplatin to be administered on Day 6, wherein the C2D1 of carboplatin is about 5×(25+creatinine clearance) mg; and
   (v) a first dose (C2D1) of etoposide to be administered on Day 6, a second dose (C2D2) of etoposide to be administered on Day 7, and a third dose (C2D3) of etoposide to be administered on Day 8, wherein the C2D1, the C2D2, and the C2D3 of etoposide are each about 100 mg/m²; and
(c) the third dosing cycle comprises:
   (i) a single dose (C3D1) of glofitamab to be administered on Day 1, wherein the C3D1 of glofitamab is about 30 mg;
   (ii) a first dose (C3D1) of rituximab to be administered on Day 5, wherein the C3D1 of rituximab is about 375 mg/m²;
   (iii) a single dose (C3D1) of ifosfamide to be administered on Day 6, wherein the C3D1 of ifosfamide is about 5000 mg/m² with a maximum dose of about 800 mg;
   (iv) a single dose (C3D1) of carboplatin to be administered on Day 6, wherein the C3D1 of carboplatin is about 5×(25+creatinine clearance) mg; and
   (v) a first dose (C3D1) of etoposide to be administered on Day 6, a second dose (C3D2) of etoposide to be administered on Day 7, and a third dose (C3D3) of etoposide to be administered on Day 8, wherein the C3D1, the C3D2, and the C3D3 of etoposide are each about 100 mg/m².

496. Use of glofitamab for treating a subject aged between 18 years and 30 years having a CD20-positive cell proliferative disorder, wherein glofitamab is to be administered in combination with obinutuzumab, rituximab, ifosfamide, carboplatin, and etoposide in a dosing regimen comprising a first dosing cycle, a second dosing cycle, and a third dosing cycle, wherein
(a) the first dosing cycle comprises:
   (i) a first dose (C1D1) of glofitamab to be administered on Day 8 and a second dose (C1D2) of glofitamab to be administered on Day 15, wherein the C1D1 of glofitamab is about 2.5 mg, and the C1D2 of glofitamab is about 10 mg;
   (ii) a first dose (C1D1) of obinutuzumab to be administered on Day 1 and a second dose (C1D2) of obinutuzumab to be administered on Day 2, wherein the C1D1 of obinutuzumab is about one-tenth the amount of the sum of the C1D1 and the C1D2 of obinutuzumab and the C1D2 of obinutuzumab is about nine-tenth the amount of the sum of the C1D1 and the C1D2 of obinutuzumab, and wherein the sum of the C1D1 and the C1D2 of obinutuzumab is about 1000 mg;
   (iii) a single dose (C1D1) of ifosfamide to be administered on Day 3, wherein the C1D1 of ifosfamide is about 5000 mg/m² with a maximum dose of about 800 mg;
   (iv) a single dose (C1D1) of carboplatin to be administered on Day 3, wherein the C1D1 of carboplatin is about 5×(25+creatinine clearance) mg; and
   (v) a first dose (C1D1) of etoposide to be administered on Day 3, a second dose (C1D2) of etoposide to be administered on Day 4, and a third dose (C1D3) of etoposide to be administered on Day 5, wherein the C1D1, the C1D2, and the C1D3 of etoposide are each about 100 mg/m²;
(b) the second dosing cycle comprises:
   (i) a single dose (C2D1) of glofitamab to be administered on Day 1, wherein the C2D1 of glofitamab is about 30 mg;
   (ii) a first dose (C2D1) of rituximab to be administered on Day 5, wherein the C2D1 of rituximab is about 375 mg/m²;
   (iii) a single dose (C2D1) of ifosfamide to be administered on Day 6, wherein the C2D1 of ifosfamide is about 5000 mg/m² with a maximum dose of about 800 mg;
   (iv) a single dose (C2D1) of carboplatin to be administered on Day 6, wherein the C2D1 of carboplatin is about 5×(25+creatinine clearance) mg; and
   (v) a first dose (C2D1) of etoposide to be administered on Day 6, a second dose (C2D2) of etoposide to be administered on Day 7, and a third dose (C2D3) of etoposide to be administered on Day 8, wherein the C2D1, the C2D2, and the C2D3 of etoposide are each about 100 mg/m²; and
(c) the third dosing cycle comprises:
   (i) a single dose (C3D1) of glofitamab to be administered on Day 1, wherein the C3D1 of glofitamab is about 30 mg;
   (ii) a first dose (C3D1) of rituximab to be administered on Day 5, wherein the C3D1 of rituximab is about 375 mg/m²;
   (iii) a single dose (C3D1) of ifosfamide to be administered on Day 6, wherein the C3D1 of ifosfamide is about 5000 mg/m² with a maximum dose of about 800 mg;
   (iv) a single dose (C3D1) of carboplatin to be administered on Day 6, wherein the C3D1 of carboplatin is about 5×(25+creatinine clearance) mg; and
   (v) a first dose (C3D1) of etoposide to be administered on Day 6, a second dose (C3D2) of etoposide to be administered on Day 7, and a third dose (C3D3) of etoposide to be administered on Day 8, wherein the C3D1, the C3D2, and the C3D3 of etoposide are each about 100 mg/m².

497. The glofitamab for use or use of any one of embodiments 485 to 496, wherein mesna is to be administered simultaneously with any dose of ifosfamide.

498. The glofitamab for use or use of embodiment 497, wherein mesna is to be administered intravenously at a dose of about 5000 mg/m².

499. The glofitamab for use or use of embodiment 498, wherein mesna is to be administered via continuous infusion over about 24 hours on Day 3 of the first dosing cycle, on Day 6 of the second dosing cycle, and/or on Day 6 of each additional dosing cycle.

500. The glofitamab for use or use of any one of embodiments 455 to 469, wherein the CD20-positive cell proliferative disorder is a relapsed and/or refractory DLBCL.

501. The glofitamab for use or use of any one of embodiments 470 to 499, wherein the CD20-positive cell proliferative disorder is a relapsed and/or refractory mature B cell NHL.

502. The invention as described hereinbefore.

### EXAMPLES

The following are examples of methods and compositions of the invention. It is understood that various other embodiments may be practiced, given the general description provided above.

### Example 1. A Phase IB, Open-Label, Multicenter, Single Arm Study Evaluating the Preliminary Efficacy, Safety, and Pharmacokinetics of Glofitamab in Combination with Rituximab Plus Ifosfamide, Carboplatin Etoposide Phosphate in Patients with Relapsed/Refractory Transplant or Car-T Therapy Eligible Diffuse B-Cell Lymphoma

### Study Rationale

The purpose of this study is to evaluate the preliminary efficacy, safety, and pharmacokinetics of glofitamab (glofit) in combination with rituximab plus ifosfamide, carboplatin, and etoposide (R-ICE) in participants with relapsed or refractory (R/R) diffuse large B-cell lymphoma (DLBCL), who have failed one prior line of therapy incorporating an anti-cluster of differentiation (CD) 20 antibody (i.e., rituximab) and an anthracycline, and who are transplant or CAR-T therapy eligible, defined as being medically eligible for intensive platinum-based salvage therapy followed by autologous stem cell transplantation (ASCT) or for CAR-T therapy.

### Objectives

The primary objective of this study is to evaluate the preliminary efficacy, safety, and pharmacokinetics of glofitamab in combination with R-ICE in participants with R/R DLBCL, who have failed one prior line of therapy incorporating an anti-CD20 antibody (i.e., rituximab) and an anthracycline, and who are transplant or CAR-T therapy eligible.

In this protocol, “study treatment” refers to the combination of treatments assigned to participants as part of this study (i.e., glofitamab and R-ICE). Specific objectives and corresponding endpoints for the study are outlined in Table 6.

TABLE 6
Objectives and Endpoints

| Objectives | Corresponding Endpoints |
|---|---|
| Primary Objectives: | |
| To evaluate the preliminary efficacy of | ORR after enrollment, defined as the |
| glofit-R-ICE | proportion of participants that |
| | achieves a CR or PR within three |
| | cycles of glofit-R-ICE, as determined |
| | by the investigator according to |
| | Lugano criteria |
| Secondary Objective: | |
| To evaluate the preliminary | EFS after enrollment, defined as the time from |
| efficacy of glofit-R-ICE | enrollment to the first occurrence of disease |
| | progression as determined by the investigator |
| | according to Lugano criteria, initiation of new anti- |
| | lymphoma therapy (not including planned ASCT |
| | or CAR-T therapy), or death from any cause |
| | (whichever occurs first) |
| | PFS after enrollment, defined as the time from |
| | enrollment to the first occurrence of disease |
| | progression or death from any cause (whichever |
| | occurs first), as determined by the investigator |
| | according to Lugano criteria |
| | MARR, defined as the proportion of participants |
| | treated with intent to proceed to ASCT that |
| | achieves a CR or PR within three cycles of glofit- |
| | R-ICE, as determined by the investigator |
| | according to Lugano criteria, and additionally |
| | achieves mobilization of a minimum of 2,000,000 |
| | CD34+ hematopoietic stem cells/kg for ASCT |
| | OS after enrollment, defined as the time from |
| | enrollment to death from any cause |
| | CR rate after enrollment, defined as the |
| | proportion of participants that achieves a CR |
| | within three cycles of glofit-R-ICE, as determined |
| | by the investigator according to Lugano criteria |
| | DOR, defined as the time from the first |
| | occurrence of a documented objective response |
| | (CR or PR) to disease progression or death from |
| | any cause (whichever occurs first), as determined |
| | by the investigator according to Lugano criteria |
| | DOCR, defined as the time from the first |
| | occurrence of a documented complete response |
| | to disease progression or death from any cause |
| | (whichever occurs first), as determined by the |
| | investigator according to Lugano criteria |
| To evaluate the safety of glofit-R-ICE | Incidence and severity of adverse events, with |
| | severity determined according to NCI CTCAE v5.0 |
| | Incidence and severity of cytokine release |
| | syndrome, with severity determined according to |
| | ASTCT 2019 criteria |
| | Change from baseline in targeted vital signs |
| | Change from baseline in targeted clinical |
| | laboratory test results |
| To establish the PK behavior of | Maximum and Minimum Serum |
| glofitamab when administered with R- | concentration of glofitamab |
| ICE | |
| To evaluate the incidence and prevalence | Prevalence of ADAs against glofitamab at |
| of ADAs to glofitamab when | baseline and incidence of ADAs during |
| administered with R-ICE | the study, and during follow up |

ADA = anti-drug antibody;
CAR-T = Chimeric Antigen Receptor T-cell;
CR = Complete Response;
CTCAE = Common Terminology Criteria for Adverse Events;
ctDNA = circulating-tumor DNA;
DOCR = duration of complete response;
DOR = duration of response;
EFS = event-free survival;
glofit = glofitamab;
MARR = mobilization adjusted response rate;
ORR = objective response rate;
OS = overall survival;
PFS = progression-free survival;
PK = pharmacokinetic;
PR = partial response;
R-ICE = Rituximab plus ifosfamide, carboplatin, etoposide
NCI CTCAE= National Cancer Institute Common Terminology Criteria for Adverse Events.

### Overall Study Design

This is a Phase Ib, open-label, multicenter, single-arm trial in patients with R/R DLBCL who have failed one prior line of therapy incorporating an anti-CD20 antibody (i.e., rituximab) and an anthracycline, and who are transplant eligible, defined as being medically eligible for intensive platinum-based salvage therapy followed by ASCT or by CAR-T therapy.

Potential participants are screened for eligibility within a 28-day screening period. Approximately 40 participants will be enrolled, through an interactive voice or web-based response system (IxRS), to receive up to three 21-day cycles of glofit-R-ICE. During Cycle 1, participants receive obinutuzumab (Gazyva®) pretreatment (Gpt) on Day 1 plus ICE on Days 1-3, followed by step up dosing of glofitamab 2.5 mg on Day 8 and 10 mg on Day 15. During Cycles 2 and 3, participants receive R-ICE between Days 1-3 and glofitamab 30 mg on Day 8.

Participants are evaluated for response after two to three cycles of glofit-R-ICE, depending on institutional standard for number of cycles. Participants who do not have evidence of disease progression after two cycles may be treated with a third cycle of glofit-R-ICE at the investigator's discretion. Participants who experience progressive disease at any time point will discontinue study therapy.

Following completion of study therapy with glofit-R-ICE, participants who achieve CR or partial response proceed to ASCT or CAR-T therapy, with specific conditioning regimen and plan of care defined per institutional standard. Participants with stable disease or progressive disease receive further therapy as defined by their treating physician. Participants are followed for survival regardless of whether they proceed to transplant, CAR-T therapy or start new anti-lymphoma therapy.

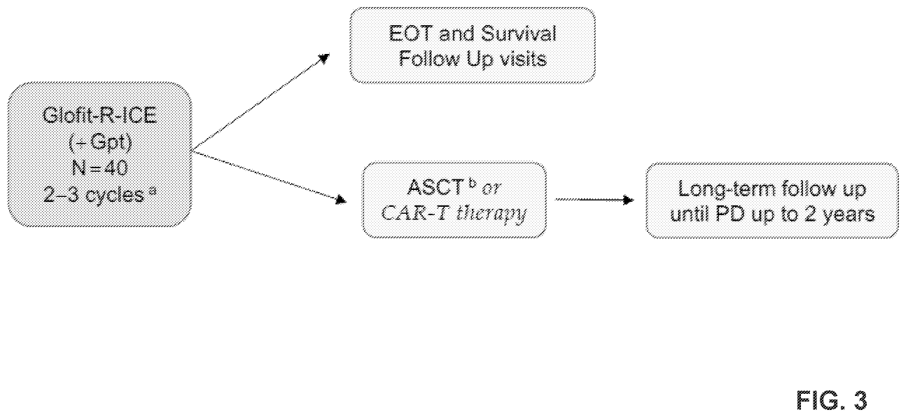

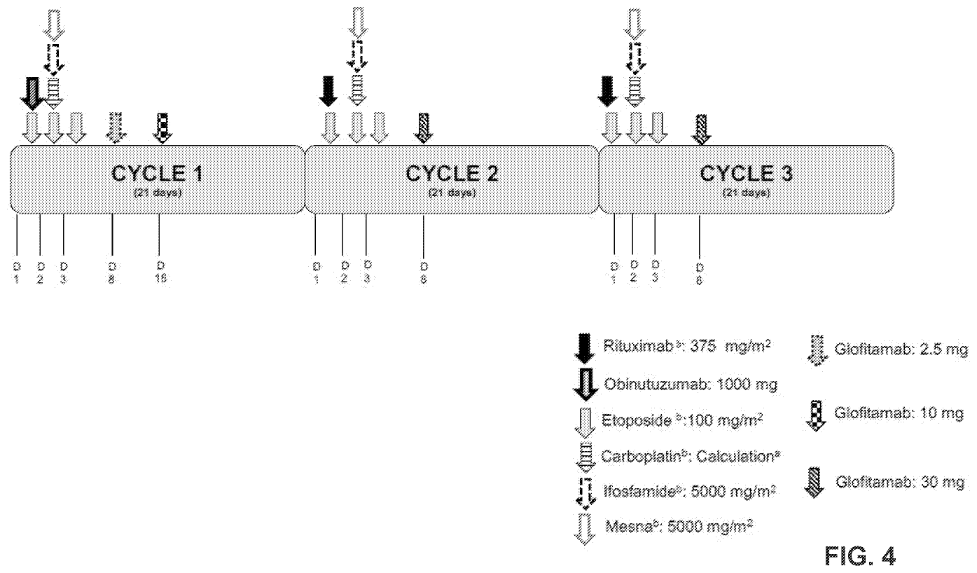

A study schema is provided in FIG. 3 and the dosing schema is shown in FIG. 4.

Study Treatment

The investigational medicinal products (IMP) for this study are glofitamab (R07082859, “glofit”), obinutuzumab (R05072759), and tocilizumab (R04877533).

Glofitamab

Glofitamab is a bispecific antibody that binds to CD20 and CD3 comprising two Fab molecules which specifically bind to CD20 comprising the following six hypervariable regions (HVRs): HVR-H1: YSWIN (SEQ ID NO: 1); HVR-H2: RIFPGDGDTDYNGKFKG (SEQ ID NO:2); HVR-H3: NVFDGYWLVY (SEQ ID NO:3); HVR-L1: RSSKSLLHSNGITYLY (SEQ ID NO: 4); HVR-L2: QMSNLVS (SEQ ID NO: 5); and HVR-L3: AQNLELPYT (SEQ ID NO: 6) and one Fab molecule which specifically binds to CD3 comprising the following six hypervariable regions (HVRs): HVR-H1: TYAMN (SEQ ID NO: 9); HVR-H2: RIRSKYNNYATYYADSVKG (SEQ ID NO:10); HVR-H3: HGNFGNSYVSWFAY (SEQ ID NO: 11); HVR-L1: GSSTGAVTTSNYAN (SEQ ID NO: 12); HVR-L2: GTNKRAP (SEQ ID NO: 13); and HVR-L3: ALWYSNLWV (SEQ ID NO: 14).

Glofitamab is administered intravenously on a step-up dosing schedule, starting with 2.5 mg on Day 8 of Cycle 1 and 10 mg on Day 15 of Cycle 1, followed by 30 mg on Day 8 of Cycles 2 and 3.

Glofitamab should be administered to well-hydrated participants. Premedication with dexamethasone 20 mg IV is required and should be administered 1 hour prior to the administration of glofitamab; premedication with oral acetaminophen or paracetamol (500 or 1000 mg) and an antihistamine, such as diphenhydramine (50 mg), should be administered approximately 30 minutes prior to the start of the infusion.

Initially, glofitamab is administered to participants over 4 hours (±15 minutes) on Days 8 and 15 of Cycle 1 and Day 8 of Cycles 2 and 3. For participants who may be at an increased risk of CRS and participants who experienced CRS with their previous dose of glofitamab or who are, in the investigator's judgment, at increased risk of recurrent CRS with subsequent doses, the time of infusion may be extended to up to 8 hours. In the absence of infusion-related adverse events from rituximab or obinutuzumab administration, or CRS from glofitamab administration, the infusion time of glofitamab in Cycle 3 may be reduced to 2 hours (±15 minutes), at the discretion of the investigator.

Obinutuzumab

Obinutuzumab pretreatment is administered by IV infusion as an absolute (flat) dose of 1000 mg on Day 1 of Cycle 1 for all participants. Obinutuzumab should be administered to well-hydrated participants.

Premedication with dexamethasone 20 mg IV or methylprednisolone 80 mg IV is required and should be administered 1 hour prior to the administration of obinutuzumab; premedication with oral acetaminophen or paracetamol (500 or 1000 mg) and an antihistamine, such as diphenhydramine (50 mg), should be administered approximately 30 minutes prior to the start of the infusion.

The obinutuzumab infusion may be split over 2 days if a participant is at increased risk for an infusion-related reaction (IRR) (e.g., because of high tumor burden, high peripheral lymphocyte count) or experiences an adverse event during the infusion.

When dexamethasone is required in the protocol, if dexamethasone is not available or if the participant has an intolerance to dexamethasone, methylprednisolone, prednisone, or prednisolone may be used. For a 20 mg IV dose of dexamethasone, an equivalent dose of 80 mg IV methylprednisolone, 100 mg prednisone PO, or 100 mg prednisolone by IV infusion may be used.

Tocilizumab

Tocilizumab is administered as a rescue investigational medicinal product (IMP) when necessary to participants who experience a CRS event. Tocilizumab is administered at dose of 8 mg/kg for participants ≥30 kg and at dose of 12 mg/kg for participants <30 kg. Doses exceeding 800 mg per infusion are not recommended.

Rituximab

Rituximab is administered intravenously at a dose of 375 mg/m² to participants on Day 1 of Cycles 2 and 3, along with ICE. Participants receive a total of 2 doses of rituximab if they complete all 3 cycles and 1 dose of rituximab if they complete just 2 cycles.

Rituximab preparation and administration are performed according to local prescribing information, including pre-medications. No subcutaneous administration of rituximab is allowed in the study. A locally approved standard of care biosimilar rituximab is permitted.

The infusion of rituximab may be split over 2 days if the participant is at increased risk for an infusion related reaction, IRR (high tumor burden or high peripheral lymphocyte count).

ICE chemotherapy (Ifosfamide, Carboplatin, and Etoposide (with Mesna))

The ICE chemotherapy consists of IV ifosfamide, carboplatin, and etoposide. Administration is permitted to be inpatient or outpatient per investigator discretion. Etoposide phosphate may be substituted for etoposide at the same dose. Ifosfamide, carboplatin, and etoposide chemotherapy will be administered beginning Day 1 of Cycle 1 for two or three 21-day cycles, as follows:
Etoposide: 100 mg/m² IV daily on Day 1, 2, and 3 of each cycle
   Participants with creatinine clearance (CrCl)<50 mL/min should be dose reduced to 75% of planned dose (or lower per institutional standard).
Carboplatin: dose in mg=5 mg/mL/min×(25+CrCl), capped at 750 mg, IV infusion on Day 2 of each cycle
   CrCl should be calculated in mL/min using Cockcroft-Gault equation:

Males: CrCl (mL/min)=[(140−age)×(weight in kg)]/[72×serum creatinine (mg/dL)]

Females: CrCl (mL/min)=0.85×CrCl (male)
Adjustments for body weight in overweight/obese participants (BMI ≥25) or for abnormally low creatinine are permitted per institutional standard.
Ifosfamide (inpatient administration): 5000 mg/m² mixed with mesna 5000 mg/m², continuous IV infusion over 24 hours starting on Day 2 of each cycle and ending on Day 3 of each cycle. Post-infusion mesna should be administered IV or PO according to institutional standard.
   Participants with CrCl<60 mL/min should be dose reduced to 80% of planned dose (or lower per institutional standard).
   Alternate dose for outpatient setting: Ifosfamide 1666 mg/m² mixed with mesna 1666 mg/m² IV daily on Day 1, 2, and 3 of each cycle is permitted if R-ICE is administered in the outpatient setting (Hertzberg et al. 2006, Ann Oncol 17, iv25-30).
      In Cycle 1, obinutuzumab should be administered prior to ICE. Initiation of ICE (Cycle 1) is permitted to be delayed one day to facilitate completion of obinutuzumab administration.
      For Cycles 2 and 3 rituximab should be administered prior to ICE. Initiation of ICE (Cycles 2 and 3) is permitted to be delayed 1 day if rituximab is administered 1 day prior to ICE per institutional standards or if there is a presentation of adverse events.
      Drug administration and monitoring for drug-related toxicity (e.g., anaphylaxis, hemorrhagic cystitis, and neurotoxicity) should be conducted according to institutional standards.

Granulocyte Colony-Stimulating Factor

All participants receive granulocyte colony-stimulating factor (G-CSF). Granulocyte colony-stimulating factor should be started 1-2 days after completion of R-ICE administration. Dosing of G-CSF should follow each site's institutional standards.

Rationale for Primary Endpoint Selection

Given that this study is a Phase Ib study evaluating the preliminary efficacy of glofit-R-ICE in participants eligible for ASCT or CAR-T therapy, that the decision on moving forward or not with transplant depends on whether the participant's disease responds to the glofit-R-ICE combination, and that superior outcomes are observed in CAR-T therapy in patients with lower disease burden, objective response rate (ORR) is a clinically meaningful endpoint for assessing preliminary activity of glofit-R-ICE. The ORR is defined as the proportion of participants that achieves a CR or PR within three cycles of glofit-R-ICE, as determined by the investigator according to Lugano criteria.

Rationale for Secondary Endpoint Selection Secondary endpoints include standard endpoints used in DLBCL to assess long term survival-based endpoints, including EFS, PFS, and OS, as well as to assess duration of overall and complete response. Additionally, an endpoint for mobilization-adjusted response rate (MARR) is included to assess the percentage of patients who achieve an objective response as well as mobilization of the target dose of 2,000,000 CD34+ hematopoietic stem cells/kg typically required as a minimum for ASCT. The definition of MARR used in this study matches the study endpoint in the CORAL study (Gisselbrecht et al. 2010, J Clin Oncol 28, 4184-90). Although no minimum dose of CD34+ hematopoietic stem cells has been defined for ASCT, practice guidelines from the American Society of Blood and Marrow Transplantation notes that a dose of 2,000,000 CD34+ hematopoietic stem cells/kg is the “generally accepted minimum” number of stem cells required for ASCT (Duong et al. 2014, Biol Bone Marrow Transplant 20, 1262-73).

Length of Study

The total duration of study participation for each individual is expected to be approximately 2 years and 6 months.

Study Population

Approximately 40 participants with DLBCL who have failed one prior line of therapy (incorporating an anti-CD20 antibody and an anthracycline) and who are transplant or CAR-T eligible will be enrolled in this study. A maximum of 20 participants diagnosed with primary R/R DLBCL within 1 year of diagnosis will be allowed in the study.

Prospective approval of protocol deviations to recruitment and enrollment criteria, also known as protocol waivers or exemptions, is not permitted.

Inclusion Criteria

Participants are eligible to be included in the study only if all of the following criteria apply:
Participants who are age ≥18 years
Life expectancy ≥12 weeks
Histologically confirmed B-cell lymphoma, including one of the following diagnoses per the 2016 WHO classification of lymphoid neoplasms that occur either de novo or as transformed from indolent lymphomas:
   DLBCL, not otherwise specified (NOS) (including Epstein-Barr virus [EBV]+DLBCL)
   High-grade B-cell lymphoma (HGBCL) with MYC and B-cell lymphoma 2 and/or B-cell lymphoma 6 rearrangements
   HGBCL, NOS
Treated with one line of prior systemic therapy including an anti-CD20 monoclonal antibody (i.e., rituximab) and an anthracycline
Relapsed or refractory disease after first-line chemoimmunotherapy
   Relapsed disease defined as complete remission to first-line therapy followed by biopsy-proven relapse
   Refractory disease defined as failure to achieve complete remission to first-line therapy, including:
      PD as best response to first-line therapy
      Stable disease (SD) as best response after at least 4 cycles of first-line therapy (e.g., 4 cycles of R-CHOP)
      PR as best response after at least 6 cycles and with either biopsy-proven residual disease or subsequent disease progression
Participant must be a candidate for high-dose chemotherapy followed by either ASCT or CAR-T therapy
At least one bi-dimensionally measurable (≥1.5 cm) nodal lesion, or one bi-dimensionally measurable ≥1 cm) extranodal lesion, as measured on CT scan
ECOG Performance Status of 0 or 1
Adequate hematologic function (unless attributable to the underlying disease, as established by extensive bone marrow involvement or associated with hypersplenism secondary to the involvement of the spleen by DLBCL per the investigator), defined as follows:
   Hemoglobin ≥9.0 g/dL (≥90 g/L)
   ANC 1.0×10⁹/L (≥1000/μL)
      Participants with history of benign ethnic neutropenia may be included with ANC≥0.75×10⁹/L (≥750/μL).
   Platelet count ≥75×10⁹/L (≥75,000/μL) without a transfusion in the week prior to starting study treatment.
Adequate renal function, defined as an estimated CrCl 45 mL/min

### Exclusion Criteria

Participants are excluded from the study if any of the following criteria apply:
Participant has been treated with more than one prior line of therapy for DLBCL.
Treatment with both partial and complete frontline regimens (i.e., rituximab, cyclophosphamide, vincristine, and prednisone followed by R-CHOP) will be counted as one line of therapy.
   Treatment with steroid monotherapy as a bridge to definitive therapy will not be counted as a prior line of therapy.
   Local therapies (e.g., radiotherapy) will not be considered as lines of therapy.
Primary mediastinal B-cell lymphoma (PMBCL)
History of severe allergic or anaphylactic reactions to humanized or murine monoclonal antibodies (or recombinant antibody-related fusion proteins) or known sensitivity or allergy to murine products
Contraindication to obinutuzumab, rituximab, ifosfamide, mesna, carboplatin, etoposide, or tocilizumab
Prior treatment with glofitamab or other bispecific antibodies targeting both CD20 and CD3
Peripheral neuropathy assessed to be Grade >1 according to National Cancer Institute Common Terminology Criteria for Adverse Events (NCI CTCAE) v5.0 at enrollment
Treatment with radiotherapy, chemotherapy, immunotherapy, immunosuppressive therapy, or any investigational agent for the purposes of treating cancer within 2 weeks prior to first study treatment
Treatment with monoclonal antibodies for the purposes of treating cancer within 4 weeks prior to first study treatment
Primary or secondary CNS lymphoma at the time of enrollment or history of CNS lymphoma
Current or history of CNS disease, such as stroke, epilepsy, CNS vasculitis, or neurodegenerative disease
   Participants with a history of stroke who have not experienced a stroke or transient ischemic attack within the past 2 years and have no residual neurologic deficits, as judged by the investigator, are allowed.
Any of the following abnormal laboratory values, unless abnormal laboratory values are associated with the underlying lymphoma per the investigator:
   AST or ALT >2.5×upper limit of normal (ULN)
   Total bilirubin 1.5×ULN
   Participants with documented Gilbert disease may be enrolled if the total bilirubin is ≤3×ULN
   INR or PT >1.5×ULN in the absence of therapeutic anticoagulation
   PTT or aPTT >1.5×ULN in the absence of therapeutic anticoagulation or a lupus anticoagulant
History of other malignancy that could affect compliance with the protocol or interpretation of results, with the following exceptions:
   Participants with a history of curatively treated basal or squamous cell carcinoma of the skin or in situ carcinoma of the cervix at any time prior to enrollment are eligible.
   Participants with low-grade, early-stage prostate cancer (Gleason score 6 or below, Stage 1 or 2) with no requirement for therapy at any time prior to enrollment are eligible.
   Participants with any other malignancy appropriately treated with curative intent and the malignancy has been in remission without treatment for 2 years prior to enrollment are eligible.
   Participants receiving adjuvant endocrine therapy for non-metastatic, hormone receptor positive breast cancer for 2 years prior to enrollment are eligible.
Evidence of significant, uncontrolled concomitant diseases that could affect compliance with the protocol or interpretation of results, including significant cardiovascular disease (such as New York Heart Association Class III or IV or Objective Assessment Class C or D cardiac disease, myocardial infarction within the last 6 months, unstable arrhythmias, or unstable angina) or significant pulmonary disease (including obstructive pulmonary disease)
Known active bacterial, viral, fungal, mycobacterial, parasitic, or other infection (excluding fungal infections of nail beds) at study enrollment or any major episode of infection (as evaluated by the investigator) within 4 weeks prior to the first study treatment
Suspected or latent tuberculosis (confirmed by positive interferon-γ release assay)
Positive test results for hepatitis B virus (HBV) infection (defined as positive hepatitis B surface antigen [HBsAg] serology)
   Participants with occult or prior HBV infection (defined as negative HBsAg and positive hepatitis B core antibody [HBcAb]) may be included if HBV DNA is undetectable, provided that they are willing to undergo DNA testing on Day 1 of Cycles 2 and 3 and every three months for at least 12 months after the last cycle of study treatment and appropriate antiviral therapy.
Positive test results for hepatitis C virus (HCV) antibody
   Participants who are positive for HCV antibody are eligible only if polymerase chain reaction (PCR) is negative for HCV RNA.
Known or suspected chronic active Epstein-Barr viral infection (CAEBV)
   CAEBV is defined as a chronic illness lasting at least 3 months with an increased EBV level in either the tissue or the blood and lack of evidence of a known underlying immunodeficiency (Kimura and Cohen 2017). Participants should not be excluded for having an EBV+DLBCL if there is no other evidence or history suggestive of CAEBV.
Known or suspected history of hemophagocytic lymphohistiocytosis (HLH)
Known history of progressive multifocal leukoencephalopathy
Adverse events from prior anti-cancer therapy that have not resolved to Grade 1 or better (with the exception of alopecia and anorexia, or as otherwise permitted by inclusion criteria)
Administration of a live, attenuated vaccine within 4 weeks before first study treatment administration or anticipation that such a live, attenuated vaccine will be required during the study
Prior solid organ transplantation
Prior allogeneic stem cell transplant
Prior ASCT for lymphoma
Prior autologous stem cell transplant for any indication other than lymphoma, within 5 years from the start of study treatment
Active autoimmune disease requiring treatment.
   Participants with a history of autoimmune-related hypothyroidism on a stable dose of thyroid-replacement hormone are eligible.
   Participants with controlled Type 1 diabetes mellitus who are on an insulin regimen are eligible for the study.
   Participants with a history of autoimmune hepatitis, systemic lupus erythematosus, inflammatory bowel disease, vascular thrombosis associated with antiphospholipid syndrome, Wegener granulomatosis, Sjögren syndrome, multiple sclerosis, or glomerulonephritis will be excluded.
   Participants with a history of immune thrombocytopenic purpura, autoimmune hemolytic anemia, Guillain-Barré syndrome, myasthenia gravis, myositis, rheumatoid arthritis, vasculitis, or other autoimmune diseases will be excluded, unless they have not required systemic therapy in the last 12 months.
Prior treatment with systemic immunosuppressive medications (including, but not limited to, cyclophosphamide, azathioprine, methotrexate, thalidomide, and anti-tumor necrosis factor agents), within 4 weeks prior to first dose of study treatment
Ongoing corticosteroid use >30 mg/day of prednisone or equivalent. Participants who received corticosteroid treatment with ≤30 mg/day of prednisone or equivalent must be documented to be on a stable dose of at least 4 weeks' duration prior to Cycle 1 Day 1. Participants may have received a brief (≤7 days) course of systemic steroids (≤100 mg prednisone equivalent per day) prior to initiation of study therapy for control of lymphoma-related symptoms.
   The use of inhaled corticosteroids is permitted.
   The use of mineralocorticoids for management of orthostatic hypotension is permitted.
   The use of physiologic doses of corticosteroids for management of adrenal insufficiency is permitted.
Recent major surgery (within 4 weeks before the first study treatment) other than for diagnosis Clinically significant history of cirrhotic liver disease
Any other diseases, metabolic dysfunction, physical examination finding, or clinical laboratory finding giving reasonable suspicion of a disease or condition that contraindicates the use of an investigational drug or that may affect the interpretation of the results or renders the participant at high risk from treatment complications
Pregnancy or breastfeeding, or intention of becoming pregnant during study treatment or within 18 months after the final dose of study treatment
   Women of childbearing potential must have a negative serum pregnancy test result within 7 days prior to initiation of study treatment.

### Example 2. A Phase I/II, Open-Label, Single-Arm, Two-Part Trial to Evaluate Safety, Tolerability, Pharmacokinetics, and Anti-Tumor Activity of Glofitamab in Combination with Chemoimmunotherapy in Pediatric and Young Adult Patients with Relapsed/Refractory Mature B-Cell Non-Hodgkin Lymphoma

### Study Rationale

The purpose of this study is to evaluate the safety and efficacy of glofitamab, a novel CD20/CD3 bispecific antibody, as monotherapy and in combination with a standard second-line chemoimmunotherapy regimen: rituximab, ifosfamide, carboplatin, and etoposide (R-ICE) in pediatric and young adult participants with relapsed and refractory (R/R) mature B-cell non-Hodgkin lymphoma (B-NHL). R/R B-NHL in children, adolescents, and young adults is an area of very high unmet need, with current 1-year overall survival (OS) rates below 30%. T-cell engagers have been identified as a medicinal product class with the greatest probability of being beneficial for the pediatric population. Combining glofitamab with R-ICE may improve response rates to salvage chemoimmunotherapy and translate into a higher percentage of participants receiving definitive therapy with HSCT and improved survival in this treatment setting.

### Objectives and Endpoints

This study evaluates the safety, tolerability, pharmacokinetics, and anti-tumor activity of glofitamab alone and in combination with R-ICE induction regimen with obinutuzumab pretreatment in pediatric and young adult participants with R/R B-NHL. Specific objectives and corresponding endpoints for the study are outlined in Table 7.

TABLE 7
Objectives and Endpoints

| Objectives | Corresponding Endpoints |
|---|---|
| Primary Objectives: | |
| To evaluate the efficacy of glofitamab in | Achievement of a CR after up to three cycles of |
| combination with R-ICE chemoimmunotherapy, as | treatment as determined by the investigator |
| assessed by the investigator | according to the International Pediatric NHL |
| | Response Criteria for pediatric participants and |
| | Lugano Classification for young adult participants |
| To evaluate the safety and tolerability of | Incidence, nature, frequency, severity, and |
| glofitamab in combination with R-ICE | timing of adverse events, with severity determined |
| chemoimmunotherapy | according to NCI CTCAE v5.0 |
| | Change from baseline in physical findings |
| | Change from baseline in targeted vital signs |
| | Change from baseline in targeted clinical |
| | laboratory test results |
| | Change from baseline in targeted ECG |
| | parameters |
| To determine the pharmacokinetics of | PK parameters (as appropriate) and serum |
| glofitamab in combination with R-ICE | concentrations of glofitamab in combination with |
| chemoimmunotherapy | R-ICE chemoimmunotherapy at specified |
| | timepoints |
| Secondary Objective: | |
| To evaluate the anti-tumor activity of glofitamab | ORR, defined as the proportion of participants |
| in combination with R-ICE chemoimmunotherapy | with a CR or PR after up to three cycles of |
| | treatment, as determined by the investigator |
| | according to the International Pediatric NHL |
| | Response Criteria for pediatric participants and |
| | Lugano Classification for young adult participants |
| | DOCR, defined as the time from the first |
| | occurrence of a documented CR until documented |
| | disease progression or death from any cause |
| | (whichever occurs first), as determined by the |
| | investigator according to the International |
| | Pediatric NHL Response Criteria for pediatric |
| | participants and Lugano Classification for young |
| | adult participants |
| | PFS after enrollment, defined as the time from |
| | enrollment to the first occurrence of disease |
| | progression or death from any cause (whichever |
| | occurs first), as determined by the investigator |
| | according to the International Pediatric NHL |
| | Response Criteria for pediatric participants and |
| | Lugano Classification for young adult participants |
| | EFS, defined as the time from enrollment to the |
| | first occurrence of disease progression, death |
| | from any cause or start of a new anti-lymphoma |
| | therapy (not including planned HSCT) |
| | OS, defined as the time from the first study |
| | treatment to the date of death from any cause |
| | Percentage of patients who proceed to HSCT |
| | after up to three cycles of treatment |
| To determine the pharmacokinetics of | Serum concentrations of obinutuzumab at |
| obinutuzumab and rituximab | specified timepoints |
| | Serum concentrations of rituximab at specified |
| | timepoints |
| To evaluate the immune response to glofitamab | Prevalence of ADAs at baseline and incidence of |
| | ADAs during the study |

ADA = anti-drug antibody;
CR = complete response;
CTCAE = Common Terminology Criteria for Adverse Events;
DOCR = duration of complete response;
EFS = event-free survival;
HSCT = hematopoietic stem cell transplantation;
NCI = National Cancer Institute;
NHL = non-Hodgkin lymphoma;
ORR = objective response rate;
OS = overall survival;
PFS = progression-free survival;
PK = pharmacokinetic;
PR = partial response;
R-ICE = rituximab, ifosfamide, carboplatin, and etoposide.
International Pediatric NHL Response Criteria: Sandlund J T, Guillerman R P, Perkins S L, et al.
International pediatric non-Hodgkin lymphoma response criteria. J. Clin. Oncol. 33: 2106-2111, 2015.
NCI CTCAE v5.0: cancer.gov

### Overall Design

This is a Phase I/II, two-part, sequential, open-label, single-arm, multicenter trial to evaluate the safety, tolerability, pharmacokinetics, and anti-tumor activity of glofitamab in combination with chemoimmunotherapy in pediatric participants from 6 months to <18 years old with R/R mature B-NHL.

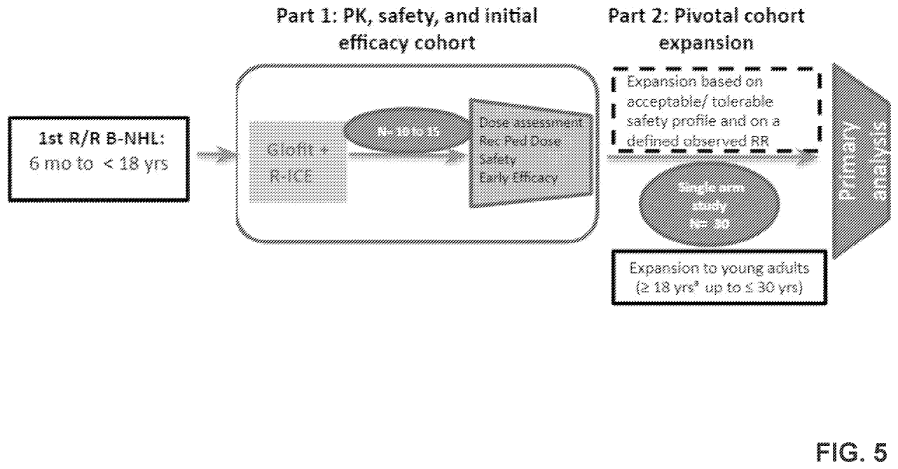

This study is divided into two parts (see FIG. 5):

Part 1—Safety Run-In:

To assess safety, tolerability, and pharmacokinetics of glofitamab in combination with R-ICE chemoimmunotherapy, to identify a recommended pediatric glofitamab dose and to evaluate early efficacy of glofitamab+R-ICE chemoimmunotherapy for R/R mature B-NHL in pediatric participants from 6 months to <18 years old, and to confirm the pediatric dose of obinutuzumab pretreatment. Between 10 and 15 participants from 6 months to <18 years old are enrolled during this portion of the study. A minimum of 10 participants are enrolled and assessed for early efficacy at the recommended pediatric dose prior to opening Part 2 of the study.

Part 2—Cohort Expansion:

If the pre-defined efficacy criteria for expansion are met and upon assessment of safety and tolerability data at the recommended pediatric glofitamab dose, a minimum of 30 new pediatric participants <18 years old are enrolled to evaluate the efficacy of glofitamab+R-ICE chemoimmunotherapy at the recommended pediatric dose of glofitamab. Eligibility for Part 2 of the study is extended to young adult participants 18-30 years old and up to an additional 10 young adults are expected to be enrolled. A maximum of 40 participants in total are enrolled in this portion of the study.

In both Parts 1 and 2, the maximum treatment duration will be three cycles of glofitamab and R-ICE.

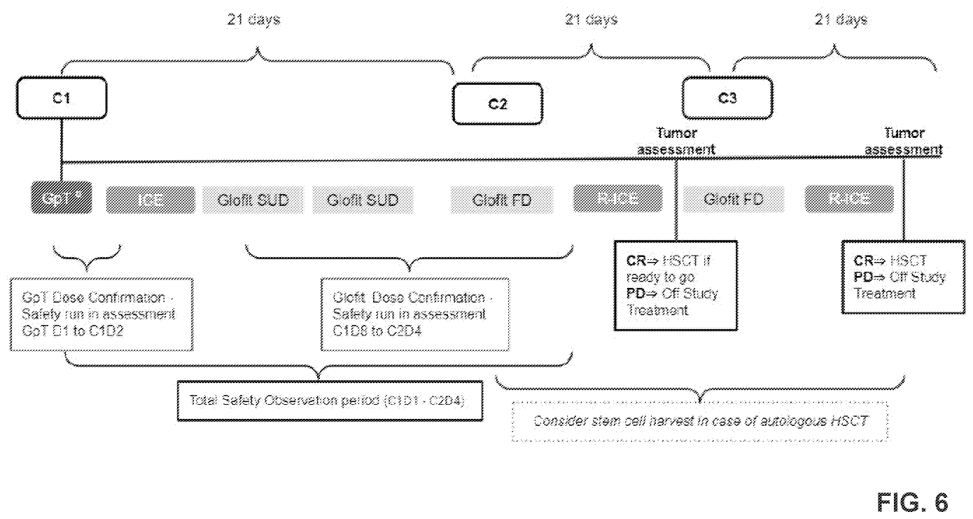

A summary of the study is shown in FIG. 6.

### Study Treatment

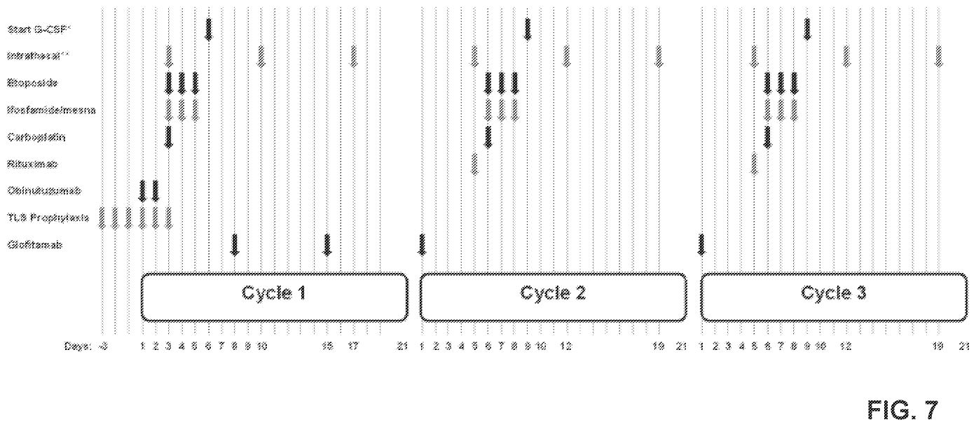

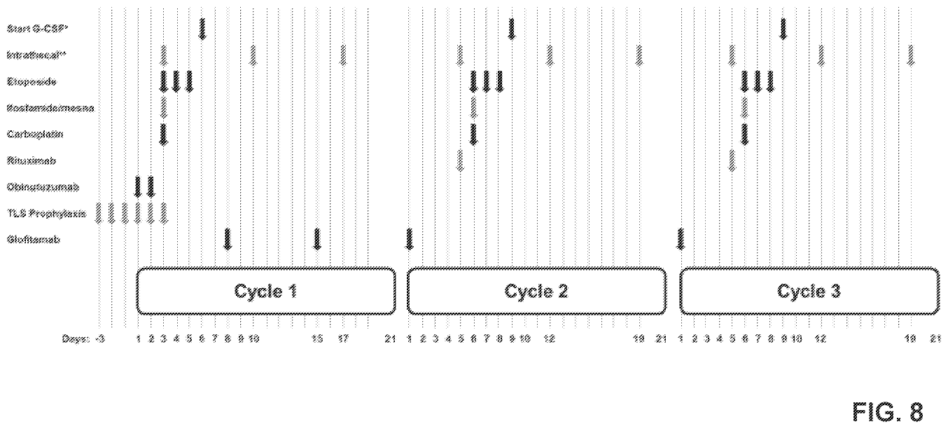

The investigational medicinal products (IMPs) for this study are obinutuzumab, glofitamab, rituximab, ifosfamide, carboplatin, etoposide (ICE), and tocilizumab, which are all administered via intravenous infusion. Other pretreatment and supportive care drugs (i.e., mesna, G-CSF, dexamethasone, and intrathecal (IT) methotrexate, cytarabine, hydrocortisone [or another steroid of equivalent dose]) are considered non-investigational medicinal products (NIMPs). Treatments are administered in accordance with FIG. 7 (pediatric patients aged 6 months to 17 years) and FIG. 8 (young adult patients aged 18 years to 30 years).

Obinutuzumab

In this study, all participants will receive one full dose of obinutuzumab pretreatment, split into two doses, during Cycle 1 and prior to starting glofitamab administration. Obinutuzumab pretreatment is administered by intravenous (IV) infusion over 4 hours only during the first dosing cycle (Cycle 1), with body weight-adjusted dosing for pediatric participants <18 years old and a flat dose of 1000 mg for participants 18-30 years old. On Day 1 of Cycle 1 (i.e., the C1D1 dose) and Day 2 of Cycle 1 (i.e., the C1D2 dose), obinutuzumab is administered as one-tenth and nine-tenth of the total dose (i.e., 100 mg for the C1D1 dose and 900 mg for the C1D2 dose), respectively. Both administrations (C1D1 dose and C1D2 dose) will be over 4 hours using the same infusion and premedication guideline. Body weight-adjusted dosing for pediatric participants <18 years old will be used (see Table 8).

TABLE 9
Weight-Adjusted Dosing for Obinutuzumab and
Glofitamab for Patients < 18 years old

| Body Weight | Obinutuzumab | Glofitamab | Glofitamab | Glofitamab |
|---|---|---|---|---|
| (kg) ^{a} | Pretreatment | First SUD | Second SUD | FD |
| 7.5 to <13 kg | 38 mg/kg | 0.04 mg/kg | 0.15 mg/kg | 0.5 mg/kg |
| ≥13 to <20 kg | 28 mg/kg | 0.03 mg/kg | 0.15 mg/kg | 0.4 mg/kg |
| ≥20 to <32 kg | 23 mg/kg | | | |
| ≥32 to <45 kg | 20 mg/kg | | | |
| ≥45 kg ^{b, c} | 1000 mg flat | 2.5 mg flat | 10 mg flat | 30 mg flat |
| | dose | dose | dose | dose |

FD = full dose; SUD = step-up dose.
^{a} Body weight ranges are based on the CDC growth charts (cdc.gov, 2000).
^{b} All participants ≥18 years old should be administered the recommended flat dosing independent of body weight.
^{c} Doses for the ≥45 kg category are based on the recommended Phase II dose in adults.

Glofitamab

Glofitamab is administered by IV infusion on a step-up dosing schedule on Day 8 of Cycle 1 and Day 15 of Cycle 1, with up to two subsequent full doses administered on Day 1 of the second dosing cycle (Cycle 2) and Day 1 of the third dosing cycle (Cycle 3).

Glofitamab is administered to participants by IV infusion using a dedicated infusion line and a catheter for the administration. First administration of glofitamab (Day 8 of Cycle 1) will be administered over 4 hours±15 minutes (see pharmacy manual for details on infusion rate). Participants in step-up dosing cohorts should also receive the Day 15 of Cycle 1 and Day 1 of Cycle 2 doses over a minimum of 4 hours. In the absence of infusion-related adverse events, the infusion time of glofitamab in subsequent cycles may be reduced to 2 hours ±15 minutes, at the discretion of the investigator. For participants who may be at an increased risk of CRS, participants who experience infusion related reactions (IRRs) or cytokine release syndrome (CRS) with their previous dose of glofitamab, or who are at increased risk of recurrent IRRs or CRS with subsequent doses, the time of infusion may be extended to up to 8 hours. All participants will be required to undergo hospitalization from Day 1 of each cycle.

Body weight-adjusted dosing for pediatric participants <18 years old will be used (see Table 8). For participants 18-30 years old, glofitamab is administered at doses independent of body weight: starting with 2.5 mg on Day 8 of Cycle 1 and 10 mg on Day 15 of Cycle 1, followed by 30 mg on Day 1 of Cycles 2 and 3.

Glofitamab full dose administration occurs on Day 1 of Cycles 2 and 3 (with a maximum delay of 7 days) and does not commence until absolute neutrophil count (ANC)>1.00×10⁹/L and platelet count >75×10⁹/L.

Rituximab

Rituximab is administered intravenously at a dose of 375 mg/m² on Day 5 of Cycles 2 and 3 for all participants. Premedication with acetaminophen and diphenhydramine is recommended. Instead of the established double dosing of rituximab on Days 1 and 3 of each R-ICE cycle in treating pediatric B-NHL, rituximab is given only on Day 5 of Cycles 2 and 3 in order to prevent an overload of CD20 receptor occupancy by three anti-CD20 antibodies and thereby potentially diminish the efficacy of glofitamab.

Premedicate with acetaminophen and diphenhydramine. Withhold rituximab if there is evidence of Hepatitis B reactivation.

First infusion:
The rate of infusion for the first hour should be 0.5 mg/kg/hr, maximum rate of 50 mg/hr for the first hour. If no infusion-related events are observed, the infusion rate can be increased by 0.5 mg/kg/hr (maximum of 50 mg/hr increase). The rate can be increased every 30 minutes until a maximum infusion rate of 400 mg/hr is reached.
In the event of any hypersensitivity or other infusion-related symptoms, the infusion should be stopped. Additional diphenhydramine may need to be given or other medications to treat the events occurring. Once the participant improves and pulmonary infiltrates and TLS have been ruled out by clinical and/or laboratory means as clinically indicated, the infusion can be restarted at 50% of the rate at which the reaction occurred.

### Subsequent Infusions:

If symptoms were encountered with the first infusion, follow the guidelines for “First Infusion” for subsequent infusions. If no symptoms were encountered, begin subsequent infusions at 1 mg/kg/hr, with a maximum rate of 50 mg/hr for the first hour. This can be increased by 1 mg/kg/hr every 30 minutes (maximum increase 50 mg/hr) to a maximum infusion rate of 400 mg/hr.
As with the initial infusion, if any infusion-related events are noted, the infusion should be stopped. Once symptoms improve, the infusion can be restarted at one-half the previous rate.

### Hypersensitivity Reactions to Rituximab:

In the event of hypersensitivity reactions to rituximab, additional doses of acetaminophen and diphenhydramine can be given. Consideration can be given to steroid treatment of reactions as well as premedication prior to subsequent doses. As transient hypotension can occur, withhold anti-hypertensive medications for 12 hours prior to rituximab administration.

Ifosfamide, Carboplatin, and Etoposide (ICE)

ICE chemotherapy consists of IV ifosfamide, carboplatin, and etoposide. Of note, during Cycle 1, ICE will be administered (omitting rituximab) between obinutuzumab pretreatment and the first glofitamab step-up dose as initial debulking therapy and additional CRS mitigation strategy. Administration of ICE begins on Day 3 of Cycle 1 for two or three 21-day cycles, as follows:
Ifosfamide
   Participants <18 years old: 3 g/m²/day IV over 2 hours daily on Days 3, 4, and 5 of Cycle 1 and Days 6, 7, and 8 of Cycles 2 and 3. See Table 10.
   Participants 18 years old: 5 g/m² continuous IV infusion (with mesna) over 24 hours starting on Day 3 of Cycle 1 and Day 6 of Cycles 2 and 3. Participants with creatinine clearance (CrCl)<60 ml/min have dose reduced to 80% of planned dose (or lower per institutional standard). See Table 11.
Carboplatin
   Participants <18 years old: 635 mg/m² (no maximum dose) IV over 1 hour on Day 3 of Cycle 1 and Day 6 of Cycles 2 and 3. See Table 10.
   Participants 18 years old: 5 mg/mL/min×(25+CrCl) IV infusion (maximum 750 mg) on Day 3 of Cycle 1 and Day 6 of Cycles 2 and 3. See Table 11.
Etoposide
   Participants <18 years old: 100 mg/m²/day IV over 1 hour daily on Days 3, 4, and 5 of Cycle 1 and Days 6, 7, and 8 of Cycles 2 and 3. See Table 10.
   Participants ≥18 years old: 100 mg/m²/day IV over 1 hour on Days 3, 4, and 5 of Cycle 1 and Days 6, 7, and 8 of Cycles 2 and 3. Participants with CrCl<50 mL/min have dose reduced to 75% of planned dose (or lower per institutional standard). See Table 11.
   Etoposide phosphate may be substituted for etoposide at the same dose.

R-ICE chemoimmunotherapy (consisting of rituximab, ifosfamide, carboplatin, and etoposide) is administered at the doses prescribed below in Tables 10 and 11. Mesna (2-mercaptoethane sulfonate Na) is administered as supportive therapy to reduce the risk of hemorrhagic cystitis known to be associated with ifosfamide.

TABLE 10
Dosing and Schedule of R-ICE Treatment Regimen for Participants <18 years old

| Drug | Dose and Schedule |
|---|---|
| Rituximab | 375 mg/m² IV on Day 5 of Cycles 2 and 3 |
| Ifosfamide | 3 g/m²/day IV over 2 hours daily on Days 3, 4, and 5 of Cycle 1, and Days 6, |
| | 7, and 8 of Cycles 2 and 3 |
| Carboplatin | 635 mg/m² IV (no maximum dose) over 1 hour on Day 3 of Cycle 1, and Day |
| | 6 of Cycles 2 and 3 |
| Etoposide | 100 mg/m²/day IV over 1 hour daily on Days 3, 4, and 5 of Cycle 1, and Days |
| | 6, 7, and 8 of Cycles 2 and 3 |
| Mesna | 600 mg/m² IV over 15 minutes before the start of ifosfamide and then at 3, 6, 9 and |
| | 12 hours after the start of ifosfamide daily on Days 3, 4, and 5 of Cycle 1, and |
| | Days 6, 7, and 8 of Cycles 2 and 3 |

IV = intravenous

TABLE 11
Dosing and Schedule of R-ICE Treatment Regimen for Participants ≥18 years old

| Drug | Dose and Schedule |
|---|---|
| Rituximab | 375 mg/m² IV on Day 5 of Cycles 2 and 3 |
| Ifosfamide | 5 g/m² continuous IV infusion (with mesna) over 24 hours on Day 3 of Cycle 1 and |
| | Day 6 of Cycles 2 and 3 |
| | Participants with CrCl < 60 ml/min ^{a} should be dose reduced to 80% of planned |
| | dose (or lower per institutional standard). |
| Carboplatin | 5 mg/mL/min x (25 + CrCl) IV infusion (maximum 750 mg) on Day 3 of Cycle 1, |
| | and Day 6 of Cycles 2 and 3 |
| Etoposide | 100 mg/m²/day IV over 1 hour on Days 3, 4, and 5 of Cycle 1, and Days 6, 7, and |
| | 8 of Cycles 2 and 3 |
| | Participants with CrCl <50 mL/min ^{a} should be dose reduced to 75% of planned |
| | dose (or lower per institutional standard). |
| Mesna | 5 g/m² continuous IV infusion (with ifosfamide) over 24 hours on Day 3 of |
| | Cycle 1 and Day 6 of Cycles 2 and 3. Post-infusion mesna should be |
| | administered IV or PO according to institutional standard. |

CrCl = creatinine clearance;
IV = intravenous,
PO = by mouth; orally.
^{a} CrCl should be calculated in mL/min using the Cockcroft-Gault equation:
Males: CrCl (mL/min) = ([140 − age] × [weight in kg])/(72 × serum creatinine [mg/dL])
Females: CrCl (mL/min) = 0.85 × CrCl (male)
Adjustments for body weight in overweight/obese participants (BMI ≥ 25) or for abnormally low creatinine are permitted per institutional standard.

A minimum of 4 days between the administration of glofitamab and the initiation of R-ICE is respected in the second and third dosing cycles. R-ICE is administered on Day 5 of the second and third dosing cycles and does not occur later than 34 days after the initiation of the previous R-ICE regimen, with the exception of treatment delays from infections in consultation with the Medical Monitor.

Mesna

Mesna is administered as described in Table 10 and Table 11. The total daily mesna dosage is equal to 100% of the daily ifosfamide dosage. For patients aged less than 18 years of age, substitution of oral mesna for the IV formulation is discouraged. Mesna dosage should be changed to a continuous 24-hour infusion in instances of microscopic or gross hematuria.

Tocilizumab

Tocilizumab is administered as a rescue investigational medicinal product (IMP) when necessary to participants who experience cytokine release syndrome. Tocilizumab is administered intravenously at a dose of 8 mg/kg for participants 30 kg and at a dose of 12 mg/kg for participants <30 kg. Doses can be repeated every 8 hours as necessary (maximum four doses), with each dose not exceeding 800 mg.

Intrathecal (IT) Chemotherapy

All intrathecal chemotherapy should be administered at least 24 hours prior to any obinutuzumab and glofitamab infusion or 48 hours after these infusions.

Participants with CNS disease (any histology) will receive IT therapy on Days 3, 10, and 17 of Cycle 1, and Days 5, 12, and 19 of Cycles 2 and 3. Participants with large B-cell lymphoma who are CNS negative will receive IT therapy on Day 3 of Cycle 1 only. Participants with Burkitt lymphoma (BL) who are CNS negative will receive IT therapy on Day 3 of Cycle 1 and Day 5 of Cycles 2 and 3. If a participant was given previous IT therapy during screening, IT therapy on Day 3 of Cycle 1 can be omitted.

IT therapy with methotrexate, cytarabine, and hydrocortisone are to be administered according to age-dependent doses (see Table 12). Depending on local guidelines, IT hydrocortisone can be substituted by another IT steroid of equivalent dose (e.g., prednisolone).

TABLE 12
IT Methotrexate, Cytarabine, and Hydrocortisone Dosing

| Age (years) | Methotrexate | Cytarabine | Hydrocortisone |
|---|---|---|---|
| 0 to <1 | 8 mg | 15 mg | 8 mg |
| 1 to <3 | 10 mg | 20 mg | 10 mg |
| 3 to <9 | 12 mg | 25 mg | 12 mg |
| ≥9 | 15 mg | 30 mg | 15 mg |

Additional Medications

As some participants may develop hypersensitivity or other IRRs to obinutuzumab or glofitamab, premedication with oral or IV acetaminophen (500-1000 mg) and an antihistamine, such as diphenhydramine (10-20 mg single dose IV [maximum single dose 1.25 mg/kg by IV infusion for children ≥2 years old; 20 mg administered rectally for children <2 years old]), must be administered at least 30 minutes prior to the start of each study drug infusion (unless contraindicated). Premedication with corticosteroids (0.15-0.5 mg/kg dexamethasone IV [maximum 10 mg daily] or a single IV dose of 1-2 mg/kg methylprednisolone, or equivalent of prednisone (100 mg or about 2 mg/kg IV) or prednisolone (100 mg or about 2 mg/kg IV)) should be administered at least 60 minutes prior to the administration of obinutuzumab and glofitamab. Corticosteroid premedication will be optional at later cycles based on investigator's assessment for participants who have tolerated the step-up doses and two full doses of glofitamab without experiencing any grade of CRS. However, if a participant experiences CRS, premedication with steroids is required to be administered for subsequent doses until no additional CRS events are observed.

Changes from this corticosteroid regimen need to be medically justified and can be implemented in consultation with the Medical Monitor. Hydrocortisone is not used as premedication. See Table 13 for details.

All obinutuzumab and glofitamab doses are administered to well-hydrated participants.

TABLE 13
Overview of Premedications before Obinutuzumab Pretreatment and Glofitamab
Infusions

| | Participants Requiring | | |
|---|---|---|---|
| Timepoint | Premedication | Premedication | Administration |
| Obinutuzumab | All participants | IV corticosteroid ^{a} | At least 60 minutes |
| pretreatment | | | prior to obinutuzumab |
| on Cycle 1, Day 1 | | | infusion |
| | | Oral or IV analgesic/ | At least 30 minutes |
| | | antipyretic medication | prior to obinutuzumab |
| | | Oral or IV | infusion |
| | | Antihistamine ^{b} | |
| | Participants at risk of TLS | Rasburicase or suitable | At least 2 days prior to |
| | (e.g., due to bulky disease | alternative along with | obinutuzumab infusion |
| | or renal impairment | adequate hydration | |
| | [CrCl < 70 mL/min]) | | |
| Glofitamab starting | All participants | IV corticosteroid ^{a, c} | At least 60 minutes |
| from Cycle 1, Day 8 | | | prior to glofitamab |
| onwards; all doses | | | infusion |
| | | Oral or IV analgesic/ | At least 30 minutes |
| | | antipyretic medication | prior to obinutuzumab |
| | | Oral or IV | infusion |
| | | Antihistamine ^{b} | |
| | Participants at risk of TLS | Rasburicase or suitable | At least 2 days prior to |
| | (e.g., due to bulky disease | alternative along with | obinutuzumab infusion |
| | or renal impairment | adequate hydration | |
| | [CrCl < 70 mL/min]) | | |

CrCl = creatinine clearance;
CRS = cytokine release syndrome;
IV = intravenous;
TLS = tumor lysis syndrome.
^{a} 0.15-0.5 mg/kg IV dexamethasone (with a maximum of 10 mg daily) or 1-2 mg/kg/day methylprednisolone or equivalent dose of IV prednisone (100 mg); hydrocortisone should not be used as it has not been effective in reducing the rate of infusion reactions. Note: The use of dexamethasone over other corticosteroids as pretreatment for glofitamab is recommended.
^{b} E.g., 10-20 mg single dose IV diphenhydramine (for children >2 years: maximum single dose 1.25 mg/kg IV diphenhydramine), unless contraindicated.
^{c} Optional at later cycles based on investigator's assessment for participants who have tolerated two target doses of glofitamab without experiencing CRS.

Granulocyte Colony-Stimulating Factor (G-CSF)

Administration of growth factor support with G-CSF is mandatory and should start 24-48 hours after the end of ICE and/or R-ICE infusion, starting on Day 6 or 7 of Cycle 1, and on Day 9 or 10 of Cycles 2 and 3. G-CSF will be administered until absolute neutrophil count (ANC) recovery with a post-nadir ANC >1.50×10⁹/L twice or >5.00×10⁹/L once. The dosage is 5 μg/kg/day IV or subcutaneously, unless peripheral blood stem cell (PBSC) collection is being prepared following Cycle 2 or Cycle 3, in which case the dose will be raised to 10 μg/kg/day.

### Schedule of Treatment Administration

See FIG. 7 for a summary of the schedule of treatment administration in patients <18 years old. See FIG. 8 for a summary of the schedule of treatment administration in patients between 18-30 years old.

For Cycles 2 and 3, glofitamab infusion occurs on Day 1 and should not commence until ANC 1.00×10⁹/L and platelet count 75×10⁹/L. Participants who achieve a complete response (CR) at the end of Cycle 2 proceeds with hematopoietic stem cell transplantation (HSCT), if possible. Such participants may also continue with Cycle 3 to bridge a longer-than-anticipated preparation time for HSCT. Participants who do not achieve CR at the end of Cycle 2 (i.e., who achieve stable disease (SD) or partial response (PR)) proceed with Cycle 3. All participants who receive Cycle 3 are reassessed at the end of Cycle 3. Participants with progressive disease after Cycle 2 or 3 come off the study. Subsequent HSCT for CR patients and subsequent potential treatment options for SD and PR patients is performed according to institutional standards. Autologous stem cell harvest in case of a planned autologous HSCT is considered during Cycle 2 or 3 and follow local guidelines.

Infection Prophylaxis

Anti-pneumocystis pneumonia prophylaxis is required in all participants, according to local/institutional guidelines.

Anti-fungal and anti-bacterial prophylaxis is required in neutropenic participants. Ciprofloxacin or other appropriate antibiotic prophylaxis for neutropenic participants are recommended and should be administered according to local/institutional guidelines until neutrophil recovery or until IV antibiotic treatment is administered (whichever occurs first).

Fungal prophylaxis for neutropenic participants is recommended and should be administered per institutional/local guidelines until neutrophil recovery or until therapeutic antifungal therapy has started.

Fluconazole and echinocandins (e.g., caspofungin, micafungin) are permitted. Strong CYP3A inhibitors, including voriconazole and posaconazole, are permitted as prophylaxis.

Duration of Participation

The total duration of study participation for each individual comprises a maximum of approximately 9 weeks of treatment (for up to three cycles), followed by subsequent follow-up assessments for at least 1 year from the end of treatment.

### Study Population

This study includes approximately 40-45 participants <18 years old and up to 10 young adult participants 18-30 years old with first R/R mature B-NHL in order to investigate glofitamab in combination with R-ICE chemoimmunotherapy and will be conducted in pediatric and young adult participants with first R/R mature B-NHL.

Inclusion Criteria:
Age 6 months to <18 years at the time of signing Informed Consent for Part 1 of the study, and age 6 months to ≤30 years old at the time of signing Informed Consent for Part 2 of the study
Histologically re-confirmed diagnosis prior to study entry of aggressive mature B-NHL (B cell non-Hodgkin's lymphoma) that expresses CD20 (reconfirmed by immunohistochemistry (IHC)), including BL (Burkitt lymphoma), BAL (mature B-cell leukemia FAB L3 or Burkitt leukemia), DLBCL (diffuse large B cell lymphoma), and PMBCL (primary medial large B cell lymphoma), at the time of first R/R (relapsed and/or refractory) disease
Refractory or relapsed disease following first-line standard-of-care chemoimmunotherapy
Measurable disease, defined as
   At least one bi-dimensionally measurable nodal lesion, defined as >1.5 cm in its longest dimension, or at least one bi-dimensionally measurable extranodal lesion, defined as >1.0 cm in its longest dimension; or
   Percentage of bone marrow involvement with lymphoma cells defined by cytomorphological analysis of bone marrow aspirates
Adequate performance status, as assessed according to the Lansky or Karnofsky Performance
Status scales:
   Participants <16 years old: Lansky Performance Status 50% (Lansky et al., Cancer. 1987)
   Participants ≥16 years old: Karnofsky Performance Status ≥50% (Karnofsky et al., Cancer. 1(4):634-656, 1948)
Adequate bone marrow function defined by the following laboratory results obtained within 7 days prior to initiation of study drug:
   Hemoglobin ≥8 g/dL (transfusion allowed)
   Peripheral absolute neutrophil count (ANC) ≥0.75×10⁹/L and no granulocyte colony-stimulating factor (G-CSF) support for at least 24 hours
   Platelet count ≥75×10⁹/L
   Participants with extensive bone marrow involvement of NHL and/or disease-related cytopenias (e.g., immune thrombocytopenia) may be enrolled if ANC ≥0.5×10 9/L for Part 1 participants. There is no minimum ANC requirement for Part 2 participants.
Platelets: not known to be refractory to platelet transfusions
Adequate liver function:
   ALT (alanine transaminase) or AST (aspartate aminotransferase) ≤3×upper limit of normal (ULN) (or ≤5×ULN for participants with concurrent liver infiltration or TLS)
   Bilirubin ≤1.5×ULN for age (or ≤2.5×ULN for participants with concurrent liver infiltration)
      Participants with documented history of Gilbert's Syndrome and in whom total bilirubin elevations are accompanied by elevated indirect bilirubin are eligible
   International normalized ratio (INR) 1.5×ULN for age
   Partial prothrombin time (PTT) or activated partial thromboplastin time (aPTT) ≤1.5×ULN for age
Adequate renal function: serum creatinine 1.5×ULN or a creatinine clearance (CrCl) of ≥50 mL/min (calculated according to the Cockcroft-Gault formula (Cockcroft and Gault, Nephron. 16(1):31-41, 1976) for participants 18 years old or Schwartz formula (Mian and Schwartz, Adv. Chronic Kidney Dis. 24(6):348-356), 2017) for participants <18 years old) for participants whose serum creatinine levels do not adequately reflect renal function, as judged by the investigator
Negative serologic or polymerase chain reaction (PCR) test results for acute or chronic hepatitis B virus (HBV) infection. Note: Participants whose HBV infection status cannot be determined by serologic test results must be negative for HBV by PCR to be eligible for study participation.
Negative test results for hepatitis C virus (HCV). Participants who are positive for HCV antibody must be negative for HCV by PCR to be eligible for study participation.

Negative test results for HIV. Individuals with a positive HIV test at screening are eligible provided they are stable on anti-retroviral therapy, have a CD4 count 200/μL, and have an undetectable viral load

Exclusion Criteria (Participants are Excluded if any of the Following Apply):
Isolated CNS disease of mature B-NHL without systemic involvement, and primary CNS lymphoma
Receipt of glofitamab prior to study enrollment
Ongoing adverse events from prior anti-cancer therapy that were not resolved to Grade ≤1. Exceptions: alopecia and Grade 2 peripheral neuropathy
Grade ≥3 adverse events, with the exception of Grade 3 endocrinopathy managed with replacement therapy
Prior solid organ transplantation
Known or suspected history of hemophagocytic lymphohistiocytosis (HLH)
Known or suspected chronic active Epstein-Barr viral infection (CAEBV). CAEBV is defined as a chronic illness lasting at least 3 months with an increased Epstein-Barr virus (EBV) level in either the tissue or the blood and lack of evidence of a known underlying immunodeficiency (Kimura and Cohen, Front. Immunol. 8:1867 2017). Participants should not be excluded for having an EBV+lymphoma if there is no other evidence or history suggestive of CAEBV.
Active autoimmune disease requiring treatment
   Participants with a history of autoimmune-related hypothyroidism on a stable dose of thyroid-replacement hormone are eligible.
   Participants with controlled Type 1 diabetes mellitus who are on an insulin regimen are eligible for the study.
   Participants with a history of autoimmune hepatitis, systemic lupus erythematosus, inflammatory bowel disease, vascular thrombosis associated with antiphospholipid syndrome, Wegener granulomatosis, Sjögren syndrome, multiple sclerosis, or glomerulonephritis will be excluded.
   Participants with a history of immune thrombocytopenic purpura, autoimmune hemolytic anemia, Guillain-Barré syndrome, myasthenia gravis, myositis, rheumatoid arthritis, vasculitis, or other autoimmune diseases will be excluded, unless they have not required systemic therapy in the last 12 months.
   Participants with eczema, psoriasis, lichen simplex chronicus, or vitiligo with dermatologic manifestations only (e.g., participants with psoriatic arthritis are excluded) are eligible for the study provided all of following conditions are met:
      Rash must cover <10% of body surface area
      Disease is well controlled at baseline and requires only low-potency topical corticosteroids
      No occurrence of acute exacerbations of the underlying condition requiring psoralen plus ultraviolet A radiation, methotrexate, retinoids, biologic agents, oral calcineurin inhibitors, or high potency oral corticosteroids within the previous 12 months
History of severe allergic or anaphylactic reactions to monoclonal antibody therapy (or recombinant antibody-related fusion proteins) or known sensitivity or allergy to murine products, except if the participant was able to safely receive it after initial administration (consider consultation with Medical Monitor)
History of confirmed progressive multifocal leukoencephalopathy
Current or past history of uncontrolled non-malignant CNS disease, such as stroke, epilepsy, CNS vasculitis, or neurodegenerative disease. Note: Participants with a history of stroke who have not experienced a stroke or transient ischemic attack in the past 2 years and have no residual neurologic deficits, as judged by the investigator, are allowed.
Evidence of significant and uncontrolled concomitant diseases that could affect compliance with the protocol or interpretation of results
Major surgery or significant traumatic injury <28 days prior to the obinutuzumab pretreatment infusion (excluding biopsies) or anticipation of the need for major surgery during study treatment
Administration of a live, attenuated vaccine within 4 weeks before the start of study treatment (obinutuzumab pretreatment) or at any time during the study treatment period and within 12 months after end of study treatment
Participants with any other diseases, metabolic dysfunction, physical examination finding, or clinical laboratory finding giving reasonable suspicion of a disease or condition that would contraindicate the use of an investigational drug
Pregnancy or breastfeeding, or intention of becoming pregnant during the study. Female participants of childbearing potential must have a negative serum pregnancy test result within 7 days prior to initiation of study treatment.
Receipt of any R-ICE chemoimmunotherapy prior to study enrollment. Systemic steroid therapy and intrathecal (IT) chemotherapy to control high tumor burden or CNS disease up to 14 days prior to start of study treatment is allowed.
Receipt of more than one prior line of standard-of-care B-NHL chemoimmunotherapy
Prior allogeneic or autologous SCT

Study Endpoints

The final analysis will be based on participant data collected through the time of study discontinuation. All analyses will be based on the safety-evaluable population. All summaries will be presented according to assigned dose level.

Primary Endpoint

The primary efficacy endpoint is investigator-assessed CR rate, defined as the proportion of participants in Cohort A (both pediatrics and young adults) whose best overall response is a CR after up to three cycles of treatment. The CR will be based on investigator assessment of PET □CT/□MRI scans using the International Pediatric NHL Response Criteria for pediatric participants <18 years old and Lugano Classification for young adult participants 18 years old and <30 years old. This analysis will be based on the efficacy-evaluable population. Participants with missing or no response assessments will be included as non-responders.

Secondary Endpoints

ORR, defined as the proportion of participants whose best overall response is a PR or a CR using the International Pediatric NHL Response Criteria for pediatric participants <18 years old (Sandlund J T, Guillerman R P, Perkins S L, et al. International pediatric non-Hodgkin lymphoma response criteria. J. Clin. Oncol. 33:2106-2111, 2015) and Lugano Classification for young adult participants 18 years old and <years old (Lugano Classification, Cheson et al. J Clin Oncol. September 20; 32(27): 3059-3067, 2014). The ORR is assessed by the investigator on PET-CT/-MRI scans after up to three cycles of treatment.

Duration of complete response (DOCR), defined as the time from the initial occurrence of a documented CR until documented disease progression or death due to any cause, whichever occurs first. DOCR is assessed by the investigator, using the International Pediatric NHL Response Criteria for pediatric participants <18 years old and Lugano Classification for young adult participants 18 years old and <30 years old.

Progression-free survival (PFS), defined as the time from the first study treatment to the first occurrence of disease progression or death from any cause, whichever occurs first. PFS will be assessed by the investigator, using the International Pediatric NHL Response Criteria for pediatric participants <18 years old and Lugano Classification for young adult participants 18 years old and <30 years old.

Event-free survival (EFS), defined as the time from the first study treatment to the first occurrence of disease progression, death from any cause, or start of a new anti-lymphoma therapy (not including planned HSCT), whichever occurs first. EFS will be assessed by the investigator, using the International Pediatric NHL Response Criteria for pediatric participants <18 years old and Lugano Classification for young adult participants 18 years old and <30 years old.

OS, defined as the time from the first study treatment to the date of death from any cause.

Percentage of patients that proceed to HSCT after up to three cycles of treatment.

### First Result

A first patient was treated in the study described herein. The 14-year old boy with first recurrence of Burkitt lymphoma achieved complete remission after two cycles chemoimmunotherapy with glofitamab and R-ICE, and will proceed to stem cell transplantation.

```
LISTING OF THE SEQUENCES
Sequence ID No: 1
Length: 5
Molecule Type: Protein
Source: Artificial Sequence
Species: Synthetic Construct
Sequence:
Tyr Ser Trp Ile Asn
1 5
Sequence ID No: 2
Length: 17
Molecule Type: Protein
Source: Artificial Sequence
Species: Synthetic Construct
Sequence:
Arg Ile Phe Pro Gly Asp Gly Asp Thr Asp Tyr Asn Gly Lys Phe Lys
1 5 10 15
Gly
Sequence ID No: 3
Length: 10
Molecule Type: Protein
Source: Artificial Sequence
Species: Synthetic Construct
Sequence:
Asn Val Phe Asp Gly Tyr Trp Leu Val Tyr
1 5 10
Sequence ID No: 4
Length: 16
Molecule Type: Protein
Source: Artificial Sequence
Species: Synthetic Construct
Sequence:
Arg Ser Ser Lys Ser Leu Leu His Ser Asn Gly Ile Thr Tyr Leu Tyr
1 5 10 15
Sequence ID No: 5
Length: 7
Molecule Type: Protein
Source: Artificial Sequence
Species: Synthetic Construct
Sequence:
Gln Met Ser Asn Leu Val Ser
1 5
Sequence ID No: 6
Length: 9
Molecule Type: Protein
Source: Artificial Sequence
Species: Synthetic Construct
Sequence:
Ala Gln Asn Leu Glu Leu Pro Tyr Thr
1 5
Sequence ID No: 7
Length: 119
Molecule Type: Protein
Source: Artificial Sequence
Species: Synthetic Construct
Sequence:
Gln Val Gln Leu Val Gln Ser Gly Ala Glu Val Lys Lys Pro Gly Ser
1 5 10 15
Ser Val Lys Val Ser Cys Lys Ala Ser Gly Tyr Ala Phe Ser Tyr Ser
                        20 25 30
Trp Ile Asn Trp Val Arg Gln Ala Pro Gly Gln Gly Leu Glu Trp Met
                35 40 45
Gly Arg Ile Phe Pro Gly Asp Gly Asp Thr Asp Tyr Asn Gly Lys Phe
        50 55 60
Lys Gly Arg Val Thr Ile Thr Ala Asp Lys Ser Thr Ser Thr Ala Tyr
65 70 75 80
Met Glu Leu Ser Ser Leu Arg Ser Glu Asp Thr Ala Val Tyr Tyr Cys
                                85 90 95
Ala Arg Asn Val Phe Asp Gly Tyr Trp Leu Val Tyr Trp Gly Gln Gly
                        100 105 110
Thr Leu Val Thr Val Ser Ser
                115
Sequence ID No: 8
Length: 112
Molecule Type: Protein
Source: Artificial Sequence
Species: Synthetic Construct
Sequence:
Asp Ile Val Met Thr Gln Thr Pro Leu Ser Leu Pro Val Thr Pro Gly
1 5 10 15
Glu Pro Ala Ser Ile Ser Cys Arg Ser Ser Lys Ser Leu Leu His Ser
                        20 25 30
Asn Gly Ile Thr Tyr Leu Tyr Trp Tyr Leu Gln Lys Pro Gly Gln Ser
                35 40 45
Pro Gln Leu Leu Ile Tyr Gln Met Ser Asn Leu Val Ser Gly Val Pro
        50 55 60
Asp Arg Phe Ser Gly Ser Gly Ser Gly Thr Asp Phe Thr Leu Lys Ile
65 70 75 80
Ser Arg Val Glu Ala Glu Asp Val Gly Val Tyr Tyr Cys Ala Gln Asn
                                85 90 95
Leu Glu Leu Pro Tyr Thr Phe Gly Gly Gly Thr Lys Val Glu Ile Lys
                        100 105 110
Sequence ID No: 9
Length: 5
Molecule Type: Protein
Source: Artificial Sequence
Species: Synthetic Construct
Sequence:
Thr Tyr Ala Met Asn
1 5
Sequence ID No: 10
Length: 19
Molecule Type: Protein
Source: Artificial Sequence
Species: Synthetic Construct
Sequence:
Arg Ile Arg Ser Lys Tyr Asn Asn Tyr Ala Thr Tyr Tyr Ala Asp Ser
1 5 10 15
Val Lys Gly
Sequence ID No: 11
Length: 14
Molecule Type: Protein
Source: Artificial Sequence
Species: Synthetic Construct
Sequence:
His Gly Asn Phe Gly Asn Ser Tyr Val Ser Trp Phe Ala Tyr
1 5 10
Sequence ID No: 12
Length: 14
Molecule Type: Protein
Source: Artificial Sequence
Species: Synthetic Construct
Sequence:
Gly Ser Ser Thr Gly Ala Val Thr Thr Ser Asn Tyr Ala Asn
1 5 10
Sequence ID No: 13
Length: 7
Molecule Type: Protein
Source: Artificial Sequence
Species: Synthetic Construct
Sequence:
Gly Thr Asn Lys Arg Ala Pro
1 5
Sequence ID No: 14
Length: 9
Molecule Type: Protein
Source: Artificial Sequence
Species: Synthetic Construct
Sequence:
Ala Leu Trp Tyr Ser Asn Leu Trp Val
1 5
Sequence ID No: 15
Length: 125
Molecule Type: Protein
Source: Artificial Sequence
Species: Synthetic Construct
Sequence:
Glu Val Gln Leu Leu Glu Ser Gly Gly Gly Leu Val Gln Pro Gly Gly
1 5 10 15
Ser Leu Arg Leu Ser Cys Ala Ala Ser Gly Phe Thr Phe Ser Thr Tyr
                        20 25 30
Ala Met Asn Trp Val Arg Gln Ala Pro Gly Lys Gly Leu Glu Trp Val
                35 40 45
Ser Arg Ile Arg Ser Lys Tyr Asn Asn Tyr Ala Thr Tyr Tyr Ala Asp
        50 55 60
Ser Val Lys Gly Arg Phe Thr Ile Ser Arg Asp Asp Ser Lys Asn Thr
65 70 75 80
Leu Tyr Leu Gln Met Asn Ser Leu Arg Ala Glu Asp Thr Ala Val Tyr
                                85 90 95
Tyr Cys Val Arg His Gly Asn Phe Gly Asn Ser Tyr Val Ser Trp Phe
                        100 105 110
Ala Tyr Trp Gly Gln Gly Thr Leu Val Thr Val Ser Ser
                115 120 125
Sequence ID No: 16
Length: 109
Molecule Type: Protein
Source: Artificial Sequence
Species: Synthetic Construct
Sequence:
Gln Ala Val Val Thr Gln Glu Pro Ser Leu Thr Val Ser Pro Gly Gly
1 5 10 15
Thr Val Thr Leu Thr Cys Gly Ser Ser Thr Gly Ala Val Thr Thr Ser
                        20 25 30
Asn Tyr Ala Asn Trp Val Gln Glu Lys Pro Gly Gln Ala Phe Arg Gly
                35 40 45
Leu Ile Gly Gly Thr Asn Lys Arg Ala Pro Gly Thr Pro Ala Arg Phe
        50 55 60
Ser Gly Ser Leu Leu Gly Gly Lys Ala Ala Leu Thr Leu Ser Gly Ala
65 70 75 80
Gln Pro Glu Asp Glu Ala Glu Tyr Tyr Cys Ala Leu Trp Tyr Ser Asn
                                85 90 95
Leu Trp Val Phe Gly Gly Gly Thr Lys Leu Thr Val Leu
                        100 105
Sequence ID No: 17
Length: 672
Molecule Type: Protein
Source: Artificial Sequence
Species: Synthetic Construct
Sequence:
Gln Val Gln Leu Val Gln Ser Gly Ala Glu Val Lys Lys Pro Gly Ser
1 5 10 15
Ser Val Lys Val Ser Cys Lys Ala Ser Gly Tyr Ala Phe Ser Tyr Ser
                        20 25 30
Trp Ile Asn Trp Val Arg Gln Ala Pro Gly Gln Gly Leu Glu Trp Met
                35 40 45
Gly Arg Ile Phe Pro Gly Asp Gly Asp Thr Asp Tyr Asn Gly Lys Phe
        50 55 60
Lys Gly Arg Val Thr Ile Thr Ala Asp Lys Ser Thr Ser Thr Ala Tyr
65 70 75 80
Met Glu Leu Ser Ser Leu Arg Ser Glu Asp Thr Ala Val Tyr Tyr Cys
                                85 90 95
Ala Arg Asn Val Phe Asp Gly Tyr Trp Leu Val Tyr Trp Gly Gln Gly
                        100 105 110
Thr Leu Val Thr Val Ser Ser Ala Ser Thr Lys Gly Pro Ser Val Phe
                115 120 125
Pro Leu Ala Pro Ser Ser Lys Ser Thr Ser Gly Gly Thr Ala Ala Leu
        130 135 140
Gly Cys Leu Val Glu Asp Tyr Phe Pro Glu Pro Val Thr Val Ser Trp
145 150 155 160
Asn Ser Gly Ala Leu Thr Ser Gly Val His Thr Phe Pro Ala Val Leu
                                165 170 175
Gln Ser Ser Gly Leu Tyr Ser Leu Ser Ser Val Val Thr Val Pro Ser
                        180 185 190
Ser Ser Leu Gly Thr Gln Thr Tyr Ile Cys Asn Val Asn His Lys Pro
                195 200 205
Ser Asn Thr Lys Val Asp Glu Lys Val Glu Pro Lys Ser Cys Asp Gly
        215 220 210
Gly Gly Gly Ser Gly Gly Gly Gly Ser Gln Ala Val Val Thr Gln Glu
225 230 235 240
Pro Ser Leu Thr Val Ser Pro Gly Gly Thr Val Thr Leu Thr Cys Gly
                                245 250 255
Ser Ser Thr Gly Ala Val Thr Thr Ser Asn Tyr Ala Asn Trp Val Gln
                        260 265 270
Glu Lys Pro Gly Gln Ala Phe Arg Gly Leu Ile Gly Gly Thr Asn Lys
                275 280 285
Arg Ala Pro Gly Thr Pro Ala Arg Phe Ser Gly Ser Leu Leu Gly Gly
        290 295 300
Lys Ala Ala Leu Thr Leu Ser Gly Ala Gln Pro Glu Asp Glu Ala Glu
305 310 315 320
Tyr Tyr Cys Ala Leu Trp Tyr Ser Asn Leu Trp Val Phe Gly Gly Gly
                                325 330 335
Thr Lys Leu Thr Val Leu Ser Ser Ala Ser Thr Lys Gly Pro Ser Val
                        340 345 350
Phe Pro Leu Ala Pro Ser Ser Lys Ser Thr Ser Gly Gly Thr Ala Ala
                355 360 365
Leu Gly Cys Leu Val Lys Asp Tyr Phe Pro Glu Pro Val Thr Val Ser
        370 375 380
Trp Asn Ser Gly Ala Leu Thr Ser Gly Val His Thr Phe Pro Ala Val
385 390 395 400
Leu Gln Ser Ser Gly Leu Tyr Ser Leu Ser Ser Val Val Thr Val Pro
                                405 410 415
Ser Ser Ser Leu Gly Thr Gln Thr Tyr Ile Cys Asn Val Asn His Lys
                        420 425 430
Pro Ser Asn Thr Lys Val Asp Lys Lys Val Glu Pro Lys Ser Cys Asp
                435 440 445
Lys Thr His Thr Cys Pro Pro Cys Pro Ala Pro Glu Ala Ala Gly Gly
        450 455 460
Pro Ser Val Phe Leu Phe Pro Pro Lys Pro Lys Asp Thr Leu Met Ile
465 470 475 480
Ser Arg Thr Pro Glu Val Thr Cys Val Val Val Asp Val Ser His Glu
                                485 490 495
Asp Pro Glu Val Lys Phe Asn Trp Tyr Val Asp Gly Val Glu Val His
                        500 505 510
Asn Ala Lys Thr Lys Pro Arg Glu Glu Gln Tyr Asn Ser Thr Tyr Arg
                515 520 525
Val Val Ser Val Leu Thr Val Leu His Gln Asp Trp Leu Asn Gly Lys
        530 535 540
Glu Tyr Lys Cys Lys Val Ser Asn Lys Ala Leu Gly Ala Pro Ile Glu
545 550 555 560
Lys Thr Ile Ser Lys Ala Lys Gly Gln Pro Arg Glu Pro Gln Val Tyr
                                565 570 575
Thr Leu Pro Pro Cys Arg Asp Glu Leu Thr Lys Asn Gln Val Ser Leu
                        580 585 590
Trp Cys Leu Val Lys Gly Phe Tyr Pro Ser Asp Ile Ala Val Glu Trp
                595 600 605
Glu Ser Asn Gly Gln Pro Glu Asn Asn Tyr Lys Thr Thr Pro Pro Val
        610 615 620
Leu Asp Ser Asp Gly Ser Phe Phe Leu Tyr Ser Lys Leu Thr Val Asp
625 630 635 640
Lys Ser Arg Trp Gln Gln Gly Asn Val Phe Ser Cys Ser Val Met His
                                645 650 655
Glu Ala Leu His Asn His Tyr Thr Gln Lys Ser Leu Ser Leu Ser Pro
                        660 665 670
Sequence ID No: 18
Length: 447
Molecule Type: Protein
Source: Artificial Sequence
Species: Synthetic Construct
Sequence:
Gln Val Gln Leu Val Gln Ser Gly Ala Glu Val Lys Lys Pro Gly Ser
1 5 10 15
Ser Val Lys Val Ser Cys Lys Ala Ser Gly Tyr Ala Phe Ser Tyr Ser
                        20 25 30
Trp Ile Asn Trp Val Arg Gln Ala Pro Gly Gln Gly Leu Glu Trp Met
                35 40 45
Gly Arg Ile Phe Pro Gly Asp Gly Asp Thr Asp Tyr Asn Gly Lys Phe
        50 55 60
Lys Gly Arg Val Thr Ile Thr Ala Asp Lys Ser Thr Ser Thr Ala Tyr
65 70 75 80
Met Glu Leu Ser Ser Leu Arg Ser Glu Asp Thr Ala Val Tyr Tyr Cys
                                85 90 95
Ala Arg Asn Val Phe Asp Gly Tyr Trp Leu Val Tyr Trp Gly Gln Gly
                        100 105 110
Thr Leu Val Thr Val Ser Ser Ala Ser Thr Lys Gly Pro Ser Val Phe
                115 120 125
Pro Leu Ala Pro Ser Ser Lys Ser Thr Ser Gly Gly Thr Ala Ala Leu
        130 135 140
Gly Cys Leu Val Glu Asp Tyr Phe Pro Glu Pro Val Thr Val Ser Trp
145 150 155 160
Asn Ser Gly Ala Leu Thr Ser Gly Val His Thr Phe Pro Ala Val Leu
                                165 170 175
Gln Ser Ser Gly Leu Tyr Ser Leu Ser Ser Val Val Thr Val Pro Ser
                        180 185 190
Ser Ser Leu Gly Thr Gln Thr Tyr Ile Cys Asn Val Asn His Lys Pro
                195 200 205
Ser Asn Thr Lys Val Asp Glu Lys Val Glu Pro Lys Ser Cys Asp Lys
        210 215 220
Thr His Thr Cys Pro Pro Cys Pro Ala Pro Glu Ala Ala Gly Gly Pro
225 230 235 240
Ser Val Phe Leu Phe Pro Pro Lys Pro Lys Asp Thr Leu Met Ile Ser
                                245 250 255
Arg Thr Pro Glu Val Thr Cys Val Val Val Asp Val Ser His Glu Asp
                        260 265 270
Pro Glu Val Lys Phe Asn Trp Tyr Val Asp Gly Val Glu Val His Asn
                275 285 280
Ala Lys Thr Lys Pro Arg Glu Glu Gln Tyr Asn Ser Thr Tyr Arg Val
        290 295 300
Val Ser Val Leu Thr Val Leu His Gln Asp Trp Leu Asn Gly Lys Glu
305 310 315 320
Tyr Lys Cys Lys Val Ser Asn Lys Ala Leu Gly Ala Pro Ile Glu Lys
                                325 330 335
Thr Ile Ser Lys Ala Lys Gly Gln Pro Arg Glu Pro Gln Val Cys Thr
                        340 345 350
Leu Pro Pro Ser Arg Asp Glu Leu Thr Lys Asn Gln Val Ser Leu Ser
                355 360 365
Cys Ala Val Lys Gly Phe Tyr Pro Ser Asp Ile Ala Val Glu Trp Glu
        370 375 380
Ser Asn Gly Gln Pro Glu Asn Asn Tyr Lys Thr Thr Pro Pro Val Leu
385 390 395 400
Asp Ser Asp Gly Ser Phe Phe Leu Val Ser Lys Leu Thr Val Asp Lys
                                405 410 415
Ser Arg Trp Gln Gln Gly Asn Val Phe Ser Cys Ser Val Met His Glu
                        420 425 430
Ala Leu His Asn His Tyr Thr Gln Lys Ser Leu Ser Leu Ser Pro
                435 440 445
Sequence ID No: 19
Length: 232
Molecule Type: Protein
Source: Artificial Sequence
Species: Synthetic Construct
Sequence:
Glu Val Gln Leu Leu Glu Ser Gly Gly Gly Leu Val Gln Pro Gly Gly
1 5 10 15
Ser Leu Arg Leu Ser Cys Ala Ala Ser Gly Phe Thr Phe Ser Thr Tyr
                        20 25 30
Ala Met Asn Trp Val Arg Gln Ala Pro Gly Lys Gly Leu Glu Trp Val
                35 40 45
Ser Arg Ile Arg Ser Lys Tyr Asn Asn Tyr Ala Thr Tyr Tyr Ala Asp
        50 55 60
Ser Val Lys Gly Arg Phe Thr Ile Ser Arg Asp Asp Ser Lys Asn Thr
65 70 75 80
Leu Tyr Leu Gln Met Asn Ser Leu Arg Ala Glu Asp Thr Ala Val Tyr
                                85 90 95
Tyr Cys Val Arg His Gly Asn Phe Gly Asn Ser Tyr Val Ser Trp Phe
                        100 105 110
Ala Tyr Trp Gly Gln Gly Thr Leu Val Thr Val Ser Ser Ala Ser Val
                115 120 125
Ala Ala Pro Ser Val Phe Ile Phe Pro Pro Ser Asp Glu Gln Leu Lys
        130 135 140
Ser Gly Thr Ala Ser Val Val Cys Leu Leu Asn Asn Phe Tyr Pro Arg
145 150 155 160
Glu Ala Lys Val Gln Trp Lys Val Asp Asn Ala Leu Gln Ser Gly Asn
                                165 170 175
Ser Gln Glu Ser Val Thr Glu Gln Asp Ser Lys Asp Ser Thr Tyr Ser
                        180 185 190
Leu Ser Ser Thr Leu Thr Leu Ser Lys Ala Asp Tyr Glu Lys His Lys
                195 200 205
Val Tyr Ala Cys Glu Val Thr His Gln Gly Leu Ser Ser Pro Val Thr
        210 215 220
Lys Ser Phe Asn Arg Gly Glu Cys
225 230
Sequence ID No: 20
Length: 219
Molecule Type: Protein
Source: Artificial Sequence
Species: Synthetic Construct
Sequence:
Asp Ile Val Met Thr Gln Thr Pro Leu Ser Leu Pro Val Thr Pro Gly
1 5 10 15
Glu Pro Ala Ser Ile Ser Cys Arg Ser Ser Lys Ser Leu Leu His Ser
                        20 25 30
Asn Gly Ile Thr Tyr Leu Tyr Trp Tyr Leu Gln Lys Pro Gly Gln Ser
                35 40 45
Pro Gln Leu Leu Ile Tyr Gln Met Ser Asn Leu Val Ser Gly Val Pro
        50 55 60
Asp Arg Phe Ser Gly Ser Gly Ser Gly Thr Asp Phe Thr Leu Lys Ile
65 70 75 80
Ser Arg Val Glu Ala Glu Asp Val Gly Val Tyr Tyr Cys Ala Gln Asn
                                85 90 95
Leu Glu Leu Pro Tyr Thr Phe Gly Gly Gly Thr Lys Val Glu Ile Lys
                        100 105 110
Arg Thr Val Ala Ala Pro Ser Val Phe Ile Phe Pro Pro Ser Asp Arg
                115 120 125
Lys Leu Lys Ser Gly Thr Ala Ser Val Val Cys Leu Leu Asn Asn Phe
        130 135 140
Tyr Pro Arg Glu Ala Lys Val Gln Trp Lys Val Asp Asn Ala Leu Gln
145 150 155 160
Ser Gly Asn Ser Gln Glu Ser Val Thr Glu Gln Asp Ser Lys Asp Ser
                                165 170 175
Thr Tyr Ser Leu Ser Ser Thr Leu Thr Leu Ser Lys Ala Asp Tyr Glu
                        180 185 190
Lys His Lys Val Tyr Ala Cys Glu Val Thr His Gln Gly Leu Ser Ser
                195 200 205
Pro Val Thr Lys Ser Phe Asn Arg Gly Glu Cys
        210 215
Sequence ID No: 21
Length: 5
Molecule Type: PRT
Source: Artificial Sequence
Species: Synthetic Construct
Sequence:
Gly Gly Gly Gly Ser
1 5
Sequence ID No: 22
Length: 5
Molecule Type: PRT
Source: Artificial Sequence
Species: Synthetic Construct
Sequence:
Ser Gly Gly Gly Gly
1 5
Sequence ID No: 23
Length: 9
Molecule Type: PRT
Source: Artificial Sequence
Species: Synthetic Construct
Sequence:
Gly Gly Gly Gly Ser Gly Gly Gly Gly
1 5
Sequence ID No: 24
Length: 20
Molecule Type: PRT
Source: Artificial Sequence
Species: Synthetic Construct
Sequence:
Gly Gly Gly Gly Ser Gly Gly Gly Gly Ser Gly Gly Gly Gly Ser Gly
1 5 10 15
Gly Gly Gly Ser
                        20
Sequence ID No: 25
Length: 12
Molecule Type: PRT
Source: Artificial Sequence
Species: Synthetic Construct
Sequence:
Gly Gly Gly Ser Gly Gly Gly Ser Gly Gly Gly Ser
1 5 10
Sequence ID No: 26
Length: 16
Molecule Type: PRT
Source: Artificial Sequence
Species: Synthetic Construct
Sequence:
Gly Gly Gly Ser Gly Gly Gly Ser Gly Gly Gly Ser Gly Gly Gly Ser
1 5 10 15
Sequence ID No: 27
Length: 20
Molecule Type: PRT
Source: Artificial Sequence
Species: Synthetic Construct
Sequence:
Gly Gly Gly Ser Gly Gly Gly Ser Gly Gly Gly Ser Gly Gly Gly Ser
1 5 10 15
Gly Gly Gly Ser
                        20
Sequence ID No: 28
Length: 24
Molecule Type: PRT
Source: Artificial Sequence
Species: Synthetic Construct
Sequence:
Gly Gly Gly Ser Gly Gly Gly Ser Gly Gly Gly Ser Gly Gly Gly Ser
1 5 10 15
Gly Gly Gly Ser Gly Gly Gly Ser
                        20
Sequence ID No: 29
Length: 10
Molecule Type: PRT
Source: Artificial Sequence
Species: Synthetic Construct
Sequence:
Gly Gly Gly Gly Ser Gly Gly Gly Gly Ser
1 5 10
Sequence ID No: 30
Length: 15
Molecule Type: PRT
Source: Artificial Sequence
Species: Synthetic Construct
Sequence:
Gly Gly Gly Gly Ser Gly Gly Gly Gly Ser Gly Gly Gly Gly Ser
1 5 10 15
Sequence ID No: 31
Length: 20
Molecule Type: PRT
Source: Artificial Sequence
Species: Synthetic Construct
Sequence:
Gly Gly Gly Gly Ser Gly Gly Gly Gly Ser Gly Gly Gly Gly Ser Gly
1 5 10 15
Gly Gly Gly Ser
                        20
Sequence ID No: 32
Length: 25
Molecule Type: PRT
Source: Artificial Sequence
Species: Synthetic Construct
Sequence:
Gly Gly Gly Gly Ser Gly Gly Gly Gly Ser Gly Gly Gly Gly Ser Gly
1 5 10 15
Gly Gly Gly Ser Gly Gly Gly Gly Ser
                        20 25
Sequence ID No: 33
Length: 13
Molecule Type: PRT
Source: Artificial Sequence
Species: Synthetic Construct
Sequence:
Gly Gly Gly Ser Gly Gly Gly Ser Gly Gly Gly Ser Gly
1 5 10
Sequence ID No: 34
Length: 17
Molecule Type: PRT
Source: Artificial Sequence
Species: Synthetic Construct
Sequence:
Gly Gly Gly Ser Gly Gly Gly Ser Gly Gly Gly Ser Gly Gly Gly Ser
1 5 10 15
Gly
Sequence ID No: 35
Length: 21
Molecule Type: PRT
Source: Artificial Sequence
Species: Synthetic Construct
Sequence:
Gly Gly Gly Ser Gly Gly Gly Ser Gly Gly Gly Ser Gly Gly Gly Ser
1 5 10 15
Gly Gly Gly Ser Gly
                        20
Sequence ID No: 36
Length: 25
Molecule Type: PRT
Source: Artificial Sequence
Species: Synthetic Construct
Sequence:
Gly Gly Gly Ser Gly Gly Gly Ser Gly Gly Gly Ser Gly Gly Gly Ser
1 5 10 15
Gly Gly Gly Ser Gly Gly Gly Ser Gly
                        20 25
Sequence ID No: 37
Length: 11
Molecule Type: PRT
Source: Artificial Sequence
Species: Synthetic Construct
Sequence:
Gly Gly Gly Gly Ser Gly Gly Gly Gly Ser Gly
1 5 10
Sequence ID No: 38
Length: 16
Molecule Type: PRT
Source: Artificial Sequence
Species: Synthetic Construct
Sequence:
Gly Gly Gly Gly Ser Gly Gly Gly Gly Ser Gly Gly Gly Gly Ser Gly
1 5 10 15
Sequence ID No: 39
Length: 21
Molecule Type: PRT
Source: Artificial Sequence
Species: Synthetic Construct
Sequence:
Gly Gly Gly Gly Ser Gly Gly Gly Gly Ser Gly Gly Gly Gly Ser Gly
1 5 10 15
Gly Gly Gly Ser Gly
                        20
Sequence ID No: 40
Length: 26
Molecule Type: PRT
Source: Artificial Sequence
Species: Synthetic Construct
Sequence:
Gly Gly Gly Gly Ser Gly Gly Gly Gly Ser Gly Gly Gly Gly Ser Gly
1 5 10 15
Gly Gly Gly Ser Gly Gly Gly Gly Ser Gly
                        20 25
Sequence ID No: 41
Length: 14
Molecule Type: PRT
Source: Artificial Sequence
Species: Synthetic Construct
Sequence:
Gly Gly Gly Ser Gly Gly Gly Ser Gly Gly Gly Ser Gly Gly
1 5 10
Sequence ID No: 42
Length: 18
Molecule Type: PRT
Source: Artificial Sequence
Species: Synthetic Construct
Sequence:
Gly Gly Gly Ser Gly Gly Gly Ser Gly Gly Gly Ser Gly Gly Gly Ser
1 5 10 15
Gly Gly
Sequence ID No: 43
Length: 22
Molecule Type: PRT
Source: Artificial Sequence
Species: Synthetic Construct
Sequence:
Gly Gly Gly Ser Gly Gly Gly Ser Gly Gly Gly Ser Gly Gly Gly Ser
1 5 10 15
Gly Gly Gly Ser Gly Gly
                        20
Sequence ID No: 44
Length: 26
Molecule Type: PRT
Source: Artificial Sequence
Species: Synthetic Construct
Sequence:
Gly Gly Gly Ser Gly Gly Gly Ser Gly Gly Gly Ser Gly Gly Gly Ser
1 5 10 15
Gly Gly Gly Ser Gly Gly Gly Ser Gly Gly
                        20 25
Sequence ID No: 45
Length: 12
Molecule Type: PRT
Source: Artificial Sequence
Species: Synthetic Construct
Sequence:
Gly Gly Gly Gly Ser Gly Gly Gly Gly Ser Gly Gly
1 5 10
Sequence ID No: 46
Length: 17
Molecule Type: PRT
Source: Artificial Sequence
Species: Synthetic Construct
Sequence:
Gly Gly Gly Gly Ser Gly Gly Gly Gly Ser Gly Gly Gly Gly Ser Gly
1 5 10 15
Gly
Sequence ID No: 47
Length: 22
Molecule Type: PRT
Source: Artificial Sequence
Species: Synthetic Construct
Sequence:
Gly Gly Gly Gly Ser Gly Gly Gly Gly Ser Gly Gly Gly Gly Ser Gly
1 5 10 15
Gly Gly Gly Ser Gly Gly
                        20
Sequence ID No: 48
Length: 27
Molecule Type: PRT
Source: Artificial Sequence
Species: Synthetic Construct
Sequence:
Gly Gly Gly Gly Ser Gly Gly Gly Gly Ser Gly Gly Gly Gly Ser Gly
1 5 10 15
Gly Gly Gly Ser Gly Gly Gly Gly Ser Gly Gly
                        20 25
Sequence ID No: 49
Length: 15
Molecule Type: PRT
Source: Artificial Sequence
Species: Synthetic Construct
Sequence:
Gly Gly Gly Ser Gly Gly Gly Ser Gly Gly Gly Ser Gly Gly Gly
1 5 10 15
Sequence ID No: 50
Length: 19
Molecule Type: PRT
Source: Artificial Sequence
Species: Synthetic Construct
Sequence:
Gly Gly Gly Ser Gly Gly Gly Ser Gly Gly Gly Ser Gly Gly Gly Ser
1 5 10 15
Gly Gly Gly
Sequence ID No: 51
Length: 23
Molecule Type: PRT
Source: Artificial Sequence
Species: Synthetic Construct
Sequence:
Gly Gly Gly Ser Gly Gly Gly Ser Gly Gly Gly Ser Gly Gly Gly Ser
1 5 10 15
Gly Gly Gly Ser Gly Gly Gly
                        20
Sequence ID No: 52
Length: 27
Molecule Type: PRT
Source: Artificial Sequence
Species: Synthetic Construct
Sequence:
Gly Gly Gly Ser Gly Gly Gly Ser Gly Gly Gly Ser Gly Gly Gly Ser
1 5 10 15
Gly Gly Gly Ser Gly Gly Gly Ser Gly Gly Gly
                        20 25
Sequence ID No: 53
Length: 13
Molecule Type: PRT
Source: Artificial Sequence
Species: Synthetic Construct
Sequence:
Gly Gly Gly Gly Ser Gly Gly Gly Gly Ser Gly Gly Gly
1 5 10
Sequence ID No: 54
Length: 18
Molecule Type: PRT
Source: Artificial Sequence
Species: Synthetic Construct
Sequence:
Gly Gly Gly Gly Ser Gly Gly Gly Gly Ser Gly Gly Gly Gly Ser Gly
1 5 10 15
Gly Gly
Sequence ID No: 55
Length: 23
Molecule Type: PRT
Source: Artificial Sequence
Species: Synthetic Construct
Sequence:
Gly Gly Gly Gly Ser Gly Gly Gly Gly Ser Gly Gly Gly Gly Ser Gly
1 5 10 15
Gly Gly Gly Ser Gly Gly Gly
                        20
Sequence ID No: 56
Length: 28
Molecule Type: PRT
Source: Artificial Sequence
Species: Synthetic Construct
Sequence:
Gly Gly Gly Gly Ser Gly Gly Gly Gly Ser Gly Gly Gly Gly Ser Gly
1 5 10 15
Gly Gly Gly Ser Gly Gly Gly Gly Ser Gly Gly Gly
                        20 25
Sequence ID No: 57
Length: 11
Molecule Type: PRT
Source: Artificial Sequence
Species: Synthetic Construct
Sequence:
Asp Gly Gly Gly Gly Ser Gly Gly Gly Gly Ser
1 5 10
```

### OTHER EMBODIMENTS

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method of treating a subject having a CD20-positive cell proliferative disorder comprising administering to the subject an effective amount of:
(a) a bispecific antibody that binds to CD20 and CD3;
(b) an anti-CD20 antibody; and
(c) one or more chemotherapeutic agents selected from ifosfamide, carboplatin, and/or etoposide in a dosing regimen comprising at least a first dosing cycle and a second dosing cycle.

2. The method of claim 1, wherein the subject is aged 18 years or older.

3. The method of claim 2, wherein the subject is aged 31 years or older.

4. The method of claim 1, wherein:
the first dosing cycle comprises a first dose (C1D1) of the bispecific antibody and a second dose (C1D2) of the bispecific antibody, wherein the C1D1 of the bispecific antibody is about 2.5 mg, and the C1D2 of the bispecific antibody is about 10 mg; and
the second dosing cycle comprises a single dose (C2D1) of the bispecific antibody, wherein the C2D1 of the bispecific antibody is about 10 mg, about 16 mg, or about 30 mg.

5. (canceled)

6. The method of claim 4, wherein:
(a) the C1D1 of the bispecific antibody and the C1D2 of the bispecific antibody are administered to the subject on Days 8 and 15, respectively, of the first dosing cycle; and/or
(b) the C2D1 of the bispecific antibody is administered to the subject on Day 8 of the second dosing cycle.

7. (canceled)

8. The method of claim 1, wherein:
(a) the anti-CD20 antibody is obinutuzumab and/or rituximab;
(b) the method further comprises administering to the subject ifosfamide, carboplatin, and etoposide;
(c) the first and second dosing cycles are each 21-day dosing cycles;
(d) the dosing regimen comprises one or more additional dosing cycles; and/or
(e) the method further comprises administering to the subject one or more additional therapeutic agents.

9. The method of claim 8, wherein:
(a) the first dosing cycle comprises a single dose (C1D1) of obinutuzumab; and the second dosing cycle comprises a single dose (C2D1) of rituximab; and/or
(b) the first dosing cycle comprises a single dose (C1D1) of ifosfamide; a single dose (C1D1) of carboplatin; and a first dose (C1D1) of etoposide, a second dose (C1D2) of etoposide, and a third dose (C1D3) of etoposide; and the second cycle comprises a single dose (C2D1) of ifosfamide; a single dose (C2D1) of carboplatin; and a first dose (C2D1) of etoposide, a second dose (C2D2) of etoposide, and a third dose (C2D3) of etoposide.

10. The method of claim 9, wherein:
(a) the C1D1 of obinutuzumab is about 1000 mg and the C2D1 of rituximab is about 375 mg/m²; and/or
(b) the anti-CD20 antibody is administered in a dosing regimen comprising at least a first dosing cycle and a second dosing cycle, wherein the first dosing cycle comprises administering to the subject the C1D1 of obinutuzumab on Day 1; and the second dosing cycle comprises administering to the subject the C2D1 of rituximab on Day 1.

11-13. (canceled)

14. The method of claim 9, wherein:
(a) ifosfamide is administered at a dose of about 5000 mg/m², about 4000 mg/m², or about 1666 mg/m², carboplatin is administered at a dose in mg to target area under the curve (AUC) of about 5 mg/mL/min with maximum dose of about 750 mg, and etoposide is administered at a dose of about 100 mg/m² or about 75 mg/m² for each dose of etoposide; and/or
(b) ifosfamide and carboplatin are administered on Day 2 of the first and second dosing cycles and the C1D1-C1D3 and C2D1-C2D3 of etoposide are administered on Days 1, 2, and 3, respectively, of the first and second dosing cycles.

15-18. (canceled)

19. The method of claim 8, wherein:
(a) the one or more additional dosing cycles are each 21-day dosing cycles; and/or
(b) the dosing regimen comprises three dosing cycles in total.

20. (canceled)

21. The method of claim 8, wherein the one or more additional dosing cycles each comprises:
(a) an additional single dose of the bispecific antibody that binds to CD20 and CD3,
(b) an additional single dose of the anti-CD20 antibody, and
(c) an additional single dose of ifosfamide, an additional single dose of carboplatin and an additional first dose, an additional second dose, and an additional third dose of etoposide.

22. The method of claim 21, wherein:
(a) the additional single dose of the bispecific antibody is about 30 mg;
(b) the additional single dose of the bispecific antibody is administered to the subject on Day 8 of each of the one or more additional dosing cycles;
(c) the anti-CD20 antibody is rituximab;
(d) the additional single dose of ifosfamide is about 5000 mg/m², about 4000 mg/m², or about 1666 mg/m², the additional single dose of carboplatin is in mg to target area under the curve (AUC) of about 5 mg/mL/min with maximum dose of about 750 mg, and the additional first dose, the additional second dose, and the additional third dose of etoposide are each about 100 mg/m² or about 75 mg/m²; and/or
(e) ifosfamide and carboplatin are administered on Day 2 of each of the one or more additional dosing cycles and the additional first dose, the additional second dose, and the additional third dose of etoposide are administered on Days 1, 2, and 3, respectively, of each of the one or more additional dosing cycles.

23-24. (canceled)

25. The method of claim 22, wherein the additional single dose of rituximab is:
(a) about 375 mg/m²; and/or
(b) administered on Day 1 of each of the one or more additional dosing cycles.

26-30. (canceled)

31. The method of claim 8, wherein the one or more additional therapeutic agents comprise:
(a) tocilizumab;
(b) a corticosteroid;
(c) an antihistamine;
(d) granulocyte-colony stimulating factor (G-CSF);
(e) an antipyretic; and/or
(f) mesna.

32. The method of claim 31, wherein:
(a) the weight of the subject is greater than or equal to about 30 kg and tocilizumab is administered at a dose of about 8 mg/kg or the weight of the subject is less than 30 kg and tocilizumab is administered at a dose of about 12 mg/kg, and wherein the maximum dose is about 800 mg;
(b) the corticosteroid comprises prednisone, prednisolone, methylprednisolone, or dexamethasone;
(c) the antihistamine is diphenhydramine;
(d) G-CSF is administered between about one day and about two days after administration of any dose of rituximab, ifosfamide, carboplatin, and/or etoposide;
(e) the antipyretic is acetaminophen or paracetamol; and/or
(f) mesna is administered:
(i) at a dose of about 5000 mg/m², about 4000 mg/m², or about 1666 mg/m² intravenously;
(ii) via continuous infusion over about 24 hours on Day 2 of each dosing cycle; and/or
(iii) simultaneously with any dose of ifosfamide.

33-35. (canceled)

36. The method of claim 32, wherein:
(a) dexamethasone is administered intravenously at a dose of about 20 mg at least about one hour prior to the administration of any dose of the bispecific antibody and/or obinutuzumab;
(b) methylprednisolone is administered intravenously at a dose of about 80 mg at least about one hour prior to the administration of any dose of the bispecific antibody and/or obinutuzumab;
(c) prednisone is administered orally at a dose of about 100 mg at least about one hour prior to the administration of any dose of the bispecific antibody;
(d) prednisolone is administered intravenously at a dose of about 100 mg at least about one hour prior to the administration of any dose of the bispecific antibody;
(e) diphenhydramine is administered orally or intravenously at a dose of about 50 mg at least about 30 minutes prior to the administration of any dose of the bispecific antibody; and/or
(f) acetaminophen or paracetamol is administered orally at a dose of between about 500 mg to about 1000 mg at least about 30 minutes prior to the administration of any dose of the bispecific antibody and/or obinutuzumab.

37-57. (canceled)

58. A method of treating a subject aged between 6 months and 17 years having a CD20-positive cell proliferative disorder comprising administering to the subject an effective amount of:
(a) a bispecific antibody that binds to CD20 and CD3;
(b) an anti-CD20 antibody; and
(c) one or more chemotherapeutic agents selected from ifosfamide, carboplatin, and/or etoposide in a dosing regimen comprising at least a first dosing cycle and a second dosing cycle.

59. The method of claim 58, wherein
the first dosing cycle comprises a first dose (C1D1) of the bispecific antibody and a second dose (C1D2) of the bispecific antibody, wherein the C1D1 of the bispecific antibody is about 0.03 mg/kg, about 0.04 mg/kg, or about 2.5 mg, and the C1D2 of the bispecific antibody is about 0.15 mg/kg or about 10 mg; and
the second dosing cycle comprises a single dose (C2D1) of the bispecific antibody, wherein the C2D1 of the bispecific antibody is about 0.4 mg/kg, about 0.5 mg/kg, or about 30 mg.

60. The method of claim 59, wherein:
(a) the subject's body weight is greater than or equal to about 7.5 kg and less than about 13 kg, and wherein the C1D1 of the bispecific antibody is about 0.04 mg/kg, the C1D2 of the bispecific antibody is about 0.15 mg/kg, and the C2D1 of the bispecific antibody is about 0.5 mg/kg;
(b) the subject's body weight is greater than or equal to about 13 kg and less than about 45 kg, and wherein the C1D1 of the bispecific antibody is about 0.03 mg/kg, the C1D2 of the bispecific antibody is about 0.15 mg/kg, and the C2D1 of the bispecific antibody is about 0.4 mg/kg; or
(c) the subject's body weight is greater than or equal to about 45 kg, and wherein the C1D1 of the bispecific antibody is about 2.5 mg, the C1D2 of the bispecific antibody is about 10 mg, and the C2D1 of the bispecific antibody is about 30 mg.

61. The method of claim 59, wherein:
(a) the C1D1 of the bispecific antibody and the C1D2 of the bispecific antibody are administered to the subject on Days 8 and 15, respectively, of the first dosing cycle; and/or
(b) the C2D1 of the bispecific antibody is administered to the subject on Day 1 of the second dosing cycle.

62. (canceled)

63. The method of claim 58, wherein:
(a) the anti-CD20 antibody is obinutuzumab and/or rituximab;
(b) the first and second dosing cycles are each 21-day dosing cycles;
(c) the dosing regimen comprises one or more additional dosing cycles;
(d) the method further comprises administering to the subject ifosfamide, carboplatin, and etoposide; and/or
(e) the method further comprises administering to the subject one or more additional therapeutic agents.

64. The method of claim 63, wherein:
(a) the first dosing cycle comprises a first dose (C1D1) of obinutuzumab and a second dose (C1D2) of obinutuzumab; and/or
(b) the second dosing cycle comprises a single dose (C2D1) of rituximab.

65. The method of claim 64, wherein:
(a) the subject's body weight is greater than or equal to about 7.5 kg and less than about 13 kg, and wherein the sum of the C1D1 and the C1D2 of obinutuzumab is about 38 mg/kg;
(b) the subject's body weight is greater than or equal to about 13 kg and less than about 20 kg, and wherein the sum of the C1D1 and the C1D2 of obinutuzumab is about 28 mg/kg;
(c) the subject's body weight is greater than or equal to about 20 kg and less than about 32 kg, and wherein the sum of the C1D1 and the C1D2 of obinutuzumab is about 23 mg/kg;
(d) the subject's body weight is greater than or equal to about 32 kg and less than about 45 kg, and wherein the sum of the C1D1 and the C1D2 of obinutuzumab is about 20 mg/kg; or
(e) the subject's body weight is greater than or equal to about 45 kg, and wherein the sum of the C1D1 and the C1D2 of obinutuzumab is about 1000 mg.

66. (canceled)

67. The method of claim 64, wherein:
(a) the subject's body weight is greater than or equal to about 7.5 kg and less than about 13 kg, and wherein the C1D1 of obinutuzumab is about 3.8 mg/kg and the C1D2 of obinutuzumab is about 34.2 mg/kg;
(b) the subject's body weight is greater than or equal to about 13 kg and less than about 20 kg, and wherein the C1D1 of obinutuzumab is about 2.8 mg/kg and the C1D2 of obinutuzumab is about 35.2 mg/kg;
(c) the subject's body weight is greater than or equal to about 20 kg and less than about 32 kg, and wherein the C1D1 of obinutuzumab is about 2.3 mg/kg and the C1D2 of obinutuzumab is about 35.7 mg/kg;
(d) the subject's body weight is greater than or equal to about 32 kg and less than about 45 kg, and wherein the C1D1 of obinutuzumab is about 2.0 mg/kg and the C1D2 of obinutuzumab is about 36.0 mg/kg; or
(e) the subject's body weight is greater than or equal to about 45 kg, and wherein the C1D1 of obinutuzumab is about 100 mg and the C1D2 of obinutuzumab is about 900 mg.

68. The method of claim 64, wherein:
(a) the C1D1 of obinutuzumab is administered to the subject on Day 1 of the first dosing cycle and the C1D2 of obinutuzumab is administered to the subject on Day 2 of the first dosing cycle;
(b) the C2D1 of rituximab is about 375 mg/m²; and/or
(c) rituximab is administered to the subject on Day 5 of the second dosing cycle.

69-72. (canceled)

73. The method of claim 63, wherein the first dosing cycle comprises:
(a) a first dose (C1D1) of ifosfamide, a second dose (C1D2) of ifosfamide, and a third dose (C1D3) of ifosfamide;
(b) a single dose (C1D1) of carboplatin; and
(c) a first dose (C1D1) of etoposide, a second dose (C1D2) of etoposide, and a third dose (C1D3) of etoposide;
and the second cycle comprises:
(a) a first dose (C2D1) of ifosfamide, a second dose (C2D2) of ifosfamide, and a third dose (C2D3) of ifosfamide;
(b) a single dose (C2D1) of carboplatin; and
(c) a first dose (C2D1) of etoposide, a second dose (C2D2) of etoposide, and a third dose (C2D3) of etoposide.

74. The method of claim 73, wherein ifosfamide is administered at a dose of about 3000 mg/m² for each dose of ifosfamide, carboplatin is administered at a dose of about 635 mg/m², and etoposide is administered at a dose of about 100 mg/m² for each dose of etoposide.

75. The method of claim 73, wherein:
(a) the C1D1, C1D2, and C1D3 of ifosfamide are administered on Days 3, 4, and 5, respectively of the first dosing cycle;
(b) the C1D1 of carboplatin is administered on Day 3 of the first dosing cycle;
(c) the C1D1, C1D2, and C1D3 of etoposide are administered on Days 3, 4, and 5, respectively, of the first dosing cycle;
(d) the C2D1, C2D2, and C2D3 of ifosfamide are administered on Days 6, 7, and 8, respectively, of the second dosing cycle;
(e) the C2D1 of carboplatin is administered on Day 6 of the second dosing cycle; and
(f) the C2D1, C2D2, and C2D3 of etoposide are administered on Days 6, 7, and 8, respectively, of the second dosing cycle.

76-77. (canceled)

78. The method of claim 63, wherein:
(a) the one or more additional dosing cycles are each 21-day dosing cycles;
(b) the dosing regimen comprises three dosing cycles in total; and/or
(c) the one or more additional dosing cycles each comprises:
(i) an additional single dose of the bispecific antibody that binds to CD20 and CD3,
(ii) an additional single dose of the anti-CD20 antibody, and
(iii) an additional first dose, an additional second dose, and an additional third dose of ifosfamide; an additional single dose of carboplatin; and an additional first dose, an additional second dose, and an additional third dose of etoposide.

79-80. (canceled)

81. The method of claim 78, wherein:
(a) the subject's body weight is greater than or equal to about 7.5 kg and less than about 13 kg, and wherein the additional single dose of the bispecific antibody is about 0.5 mg/kg;
(b) the subject's body weight is greater than or equal to about 13 kg and less than about 45 kg, and wherein the additional single dose of the bispecific antibody is about 0.4 mg/kg; or
(c) the subject's body weight is greater than or equal to about 45 kg, and wherein the additional single dose of the bispecific antibody is about 30 mg.

82. The method of claim 78, wherein:
(a) the additional single dose of the bispecific antibody is administered to the subject on Day 1 of each of the one or more additional dosing cycles; and/or
(b) the anti-CD20 antibody is rituximab.

83. (canceled)

84. The method of claim 82, wherein:
(a) the additional single dose of rituximab is about 375 mg/m²; and/or
(b) the additional single dose of rituximab is administered on Day 5 of each of the one or more additional dosing cycles.

85. (canceled)

86. The method of claim 78, wherein the additional first dose, additional second dose, and additional third dose of ifosfamide are each about 3000 mg/m², the additional single dose of carboplatin is about 635 mg/m², and the additional first dose, the additional second dose, and the additional third dose of etoposide are each about 100 mg/m².

87. The method of claim 78, wherein:
(a) the additional first dose, the additional second dose, and the additional third dose of ifosfamide are administered to the subject on Days 6, 7, and 8, respectively, of each of the one or more additional dosing cycles;
(b) the additional single dose of carboplatin is administered on Day 6 of each of the one or more additional dosing cycles; and
(c) the additional first dose, the additional second dose, and the additional third dose of etoposide are administered to the subject on Days 6, 7, and 8, respectively, of each of the one or more additional dosing cycles.

88. (canceled)

89. The method of claim 63, wherein the one or more additional therapeutic agents comprise:
(a) tocilizumab;
(b) a corticosteroid;
(c) an antihistamine;
(d) granulocyte-colony stimulating factor (G-CSF);
(e) an antipyretic; and/or
(f) mesna.

90. The method of claim 89, wherein:
(a) the weight of the subject is greater than or equal to about 30 kg and tocilizumab is administered at a dose of about 8 mg/kg or the weight of the subject is less than 30 kg and tocilizumab is administered at a dose of about 12 mg/kg, and wherein the maximum dose is about 800 mg;
(b) the corticosteroid comprises prednisone, prednisolone, methylprednisolone, or dexamethasone;
(c) the antihistamine is diphenhydramine;
(d) G-CSF is administered:
(i) between about one day and about two days after administration of any dose of rituximab, ifosfamide, carboplatin, and/or etoposide; and/or
(ii) intravenously or subcutaneously at a dose of about 5 μg/kg/day or about 10 μg/kg/day;
(e) the antipyretic is acetaminophen or paracetamol; and/or
(f) mesna is administered intravenously daily as five doses totaling 3000 mg/m² in amount.

91-93. (canceled)

94. The method of claim 90, wherein:
(a) dexamethasone is administered intravenously at a dose of between about 0.15 mg/kg to about mg/kg at least about one hour prior to the administration of any dose of the bispecific antibody and/or obinutuzumab, and wherein the maximum daily dose is 10 mg;
(b) methylprednisolone is administered intravenously at a dose of between about 1 mg/kg to about 2 mg/kg at least about one hour prior to the administration of any dose of the bispecific antibody and/or obinutuzumab;
(c) prednisone or prednisolone is administered intravenously at a dose of about 100 mg or about 2 mg/kg at least about one hour prior to the administration of any dose of the bispecific antibody and/or obinutuzumab;
(d) G-CSF is administered at a dose of about 5 μg/kg/day in the first dosing cycle and about 10 μg/kg/day in the second dosing cycle and/or each additional dosing cycle;
(e) acetaminophen or paracetamol is administered:
(i) orally or intravenously at a dose of between about 500 to about 1000 mg; and/or
(ii) at least about 30 minutes prior to the administration of any dose of the bispecific antibody and/or the anti-CD20 antibody; and/or
(f) mesna is administered:
(i) intravenously at a first dose of about 600 mg/m² prior to the administration of any dose of ifosfamide and at four repeated doses of about 600 mg/m² each at about three hours, about six hours, about nine hours, and about 12 hours, respectively, after the first dose of ifosfamide; and/or
(ii) daily to the subject on Days 3, 4, and 5 of the first dosing cycle, on Days 6, 7, and 8 of the second dosing cycle, and/or on Days 6, 7, and 8 of each additional dosing cycle.

95-103. (canceled)

104. The method of claim 90, wherein the subject:
(a) is aged between two years and 17 years, and wherein diphenhydramine is administered intravenously at a dose of between about 10 mg to 20 mg with a maximum single dose of about 1.25 mg/kg; or
(b) aged less than two years, and wherein diphenhydramine is administered rectally at a dose of about 20 mg.

105. (canceled)

106. The method of claim 104, wherein diphenhydramine is administered at least about 30 minutes prior to the administration of any dose of the bispecific antibody and/or the anti-CD20 antibody.

107-118. (canceled)

119. A method of treating a subject aged between 18 years and 30 years having a CD20-positive cell proliferative disorder comprising administering to the subject an effective amount of:
(a) a bispecific antibody that binds to CD20 and CD3;
(b) an anti-CD20 antibody; and
(c) one or more chemotherapeutic agents selected from ifosfamide, carboplatin, and/or etoposide in a dosing regimen comprising at least a first dosing cycle and a second dosing cycle.

120. The method of claim 119, wherein
the first dosing cycle comprises a first dose (C1D1) of the bispecific antibody and a second dose (C1D2) of the bispecific antibody, wherein the C1D1 of the bispecific antibody is about 2.5 mg, and the C1D2 of the bispecific antibody is about 10 mg; and
the second dosing cycle comprises a single dose (C2D1) of the bispecific antibody, wherein the C2D1 of the bispecific antibody is about 30 mg.

121. The method of claim 120, wherein:
(a) the C1D1 of the bispecific antibody and the C1D2 of the bispecific antibody are administered to the subject on Days 8 and 15, respectively, of the first dosing cycle; and/or
(b) the C2D1 of the bispecific antibody is administered to the subject on Day 1 of the second dosing cycle.

122. (canceled)

123. The method of claim 119, wherein:
(a) the anti-CD20 antibody is obinutuzumab and/or rituximab,
(b) the first and second dosing cycles are each 21-day dosing cycles;
(c) the dosing regimen comprises one or more additional dosing cycles;
(d) the method further comprises administering to the subject ifosfamide, carboplatin, and etoposide; and/or
(e) the method further comprises administering to the subject one or more additional therapeutic agents.

124. The method of claim 123, wherein:
(a) the first dosing cycle comprises a first dose (C1D1) of obinutuzumab and a second dose (C1D2) of obinutuzumab; and/or
(b) the second dosing cycle comprises a single dose (C2D1) of rituximab.

125. The method of claim 124, wherein:
(a) the sum of the C1D1 and the C1D2 of obinutuzumab is about 1000 mg;
(b) the C1D1 of obinutuzumab is administered to the subject on Day 1 of the first dosing cycle and the C1D2 of obinutuzumab is administered to the subject on Day 2 of the first dosing cycle;
(c) the C2D1 of rituximab is about 375 mg/m²; and/or
(d) the C2D1 of rituximab is administered to the subject on Day 5 of the second dosing cycle.

126. (canceled)

127. The method of claim 124, wherein the C1D1 of obinutuzumab is about 100 mg and the C1D2 of obinutuzumab is about 900 mg.

128-132. (canceled)

133. The method of claim 123, wherein the first dosing cycle comprises:
(a) a single dose (C1D1) of ifosfamide;
(b) a single dose (C1D1) of carboplatin; and
(c) a first dose (C1D1) of etoposide, a second dose (C1D2) of etoposide, and a third dose (C1D3) of etoposide;
and the second cycle comprises:
(a) a single dose (C2D1) of ifosfamide;
(b) a single dose (C2D1) of carboplatin; and
(c) a first dose (C2D1) of etoposide, a second dose (C2D2) of etoposide, and a third dose (C2D3) of etoposide.

134. The method of claim 133, wherein ifosfamide is administered at a dose of about 5000 mg/m², carboplatin is administered at a dose of about 5×(25+CreatinineClearance (CrCl)) mg with maximum dose of about 750 mg, and etoposide is administered at a dose of about 100 mg/m² for each dose of etoposide.

135. The method of claim 134, wherein:
(a) the subject is male, and wherein CrCl is calculated using the formula CrCl=([140−age]×[weight in kg])/(72×[serum creatinine in mg/dL]); or
(b) the subject is female, and wherein CrCl is calculated using the formula CrCl=0.85×([140−age]×[weight in kg])/(72×[serum creatinine in mg/dL]).

136. The method of claim 134, wherein:
(a) the subject has CrCl<about 60 mL/min, and wherein each single dose of ifosfamide is reduced to 4000 mg/m²; and/or
(b) the subject has CrCl<about 50 mL/min, and wherein each dose of etoposide is reduced to about 75 mg/m².

137. The method of claim 133, wherein:
(a) the C1D1 ifosfamide is administered on Day 3 of the first dosing cycle;
(b) the C1D1 of carboplatin is administered on Day 3 of the first dosing cycle;
(c) the C1D1, C1D2, and C1D3 of etoposide are administered on Days 3, 4, and 5, respectively, of the first dosing cycle;
(d) the C2D1 of ifosfamide is administered on Day 6 of the second dosing cycle;
(e) the C2D1 of carboplatin is administered on Day 6 of the second dosing cycle; and
(f) the C2D1, C2D2, and C2D3 of etoposide are administered on Days 6, 7, and 8, respectively, of the second dosing cycle.

138-139. (canceled)

140. The method of claim 123, wherein:
(a) the one or more additional dosing cycles are each 21-day dosing cycles;
(b) the dosing regimen comprises three dosing cycles in total; and/or
(c) the one or more additional dosing cycles each comprise:
(i) an additional single dose of the bispecific antibody that binds to CD20 and CD3,
(ii) an additional single dose of the anti-CD20 antibody, and
(iii) an additional single dose of ifosfamide; an additional single dose of carboplatin; and an additional first dose, an additional second dose, and an additional third dose of etoposide.

141-142. (canceled)

143. The method of claim 140, wherein:
(a) the additional single dose of the bispecific antibody is about 30 mg,
(b) the additional single dose of the bispecific antibody is administered to the subject on Day 1 of each of the one or more additional dosing cycles; and/or
(c) the anti-CD20 antibody is rituximab.

144-145. (canceled)

146. The method of claim 143, wherein the additional single dose of rituximab is:
(a) about 375 mg/m²; and/or
(b) administered on Day 5 of each of the one or more additional dosing cycles.

147. (canceled)

148. The method of claim 140, wherein the additional single dose of ifosfamide is about 5000 mg/m², the additional single dose of carboplatin is about 5×(25+CreatinineClearance (CrCl)) mg with maximum dose of about 750 mg, and the additional first dose, the additional second dose, and the additional third dose of etoposide are each about 100 mg/m².

149. The method of claim 148, wherein:
(a) the subject is male, and wherein CrCl is calculated using the formula CrCl=([140−age]×[weight in kg])/(72×[serum creatinine in mg/dL]); or
(b) the subject is female, and wherein CrCl is calculated using the formula CrCl=0.85×([140−age]×[weight in kg])/(72×[serum creatinine in mg/dL]).

150. The method of claim 148, wherein:
(a) the subject has CrCl<about 60 mL/min, and wherein the additional single dose of ifosfamide is reduced to 4000 mg/m²; and/or
(b) the subject has CrCl<about 50 mL/min, and wherein each additional dose of etoposide is reduced to about 75 mg/m².

151. The method of claim 140, wherein:
(a) the additional single dose of ifosfamide is administered on Day 6 of each of the one or more additional dosing cycles;
(b) the additional single dose of carboplatin is administered on Day 6 of each of the one or more additional dosing cycles; and
(c) the additional first dose, the additional second dose, and the additional third dose of etoposide are administered to the subject on Days 6, 7, and 8, respectively, of each of the one or more additional dosing cycles.

152. (canceled)

153. The method of claim 123, wherein the one or more additional therapeutic agents comprise:
(a) tocilizumab;
(b) a corticosteroid;
(c) an antihistamine;
(d) granulocyte-colony stimulating factor (G-CSF);
(e) an antipyretic; and/or
(f) mesna.

154. The method of claim 153, wherein:
(a) the weight of the subject is greater than or equal to about 30 kg and tocilizumab is administered at a dose of about 8 mg/kg or the weight of the subject is less than 30 kg and tocilizumab is administered at a dose of about 12 mg/kg, and wherein the maximum dose is about 800 mg;
(b) the corticosteroid comprises prednisone, prednisolone, methylprednisolone, or dexamethasone;
(c) the antihistamine is diphenhydramine;
(d) G-CSF is administered:
(i) between about one day and about two days after administration of any dose of rituximab, ifosfamide, carboplatin, and/or etoposide; and/or
(ii) intravenously or subcutaneously at a dose of about 5 μg/kg/day or about 10 μg/kg/day;
(e) the antipyretic is acetaminophen or paracetamol; and/or
(f) mesna is administered intravenously at a dose of about 5000 mg/m².

155-157. (canceled)

158. The method of claim 154, wherein:
(a) dexamethasone is administered intravenously at a dose of between about 0.15 mg/kg to about mg/kg at least about one hour prior to the administration of any dose of the bispecific antibody and/or obinutuzumab, and wherein the maximum daily dose is 10 mg;
(b) wherein methylprednisolone is administered intravenously at a dose of between about 1 mg/kg to about 2 mg/kg at least about one hour prior to the administration of any dose of the bispecific antibody and/or obinutuzumab;
(c) prednisone or prednisolone is administered intravenously at a dose of about 100 mg or about 2 mg/kg at least about one hour prior to the administration of any dose of the bispecific antibody and/or obinutuzumab;
(d) diphenhydramine is administered:
(i) orally or intravenously at a dose of about 50 mg; and/or
(ii) at least about 30 minutes prior to the administration of any dose of the bispecific antibody and/or the anti-CD20 antibody;
(e) G-CSF is administered at a dose of about 5 μg/kg/day in the first dosing cycle and about 10 μg/kg/day in the second dosing cycle and/or each additional dosing cycle;
(f) acetaminophen or paracetamol is administered:
(i) orally or intravenously at a dose of between about 500 to about 1000 mg; and/or
(ii) at least about 30 minutes prior to the administration of any dose of the bispecific antibody and/or the anti-CD20 antibody; and/or
(g) mesna is administered:
(i) via continuous infusion over about 24 hours on Day 3 of the first dosing cycle, on Day 6 of the second dosing cycle, and/or on Day 6 of each additional dosing cycle; and/or
(ii) simultaneously with any dose of ifosfamide.

159-181. (canceled)

182. The method of claim 1, wherein the bispecific antibody comprises at least one Fab molecule which specifically binds to CD20 comprising the following six hypervariable regions (HVRs):
(i) an HVR-H1 comprising the amino acid sequence of YSWIN (SEQ ID NO: 1);
(ii) an HVR-H2 comprising the amino acid sequence of RIFPGDGDTDYNGKFKG (SEQ ID NO:2);
(iii) an HVR-H3 comprising the amino acid sequence of NVFDGYWLVY (SEQ ID NO: 3);
(iv) an HVR-L1 comprising the amino acid sequence of RSSKSLLHSNGITYLY (SEQ ID NO: 4);
(v) an HVR-L2 comprising the amino acid sequence of QMSNLVS (SEQ ID NO: 5); and
(vi) an HVR-L3 comprising the amino acid sequence of AQNLELPYT (SEQ ID NO: 6).

183. The method of claim 182, wherein the bispecific antibody comprises at least one Fab molecule which specifically binds to CD20 comprising (a) a heavy chain variable (VH) domain comprising an amino acid sequence having at least 95% sequence identity to the amino acid sequence of SEQ ID NO: 7; (b) a light chain variable (VL) domain comprising an amino acid sequence having at least 95% sequence identity to the amino acid sequence of SEQ ID NO: 8; or (c) a VH domain as in (a) and a VL domain as in (b).

184. The method of claim 183, wherein the Fab molecule which specifically binds to CD20 comprises (a) a VH domain comprising the amino acid sequence of SEQ ID NO: 7 and (b) a VL domain comprising the amino acid sequence of SEQ ID NO: 8.

185. The method of claim 1, wherein the bispecific antibody comprises at least one Fab molecule which specifically binds to CD3 comprising the following six HVRs:
(i) an HVR-H1 comprising the amino acid sequence of TYAMN (SEQ ID NO: 9);
(ii) an HVR-H2 comprising the amino acid sequence of RIRSKYNNYATYYADSVKG (SEQ ID NO: 10);
(iii) an HVR-H3 comprising the amino acid sequence of HGNFGNSYVSWFAY (SEQ ID NO: 11);
(iv) an HVR-L1 comprising the amino acid sequence of GSSTGAVTTSNYAN (SEQ ID NO: 12);
(v) an HVR-L2 comprising the amino acid sequence of GTNKRAP (SEQ ID NO: 13); and
(vi) an HVR-L3 comprising the amino acid sequence of ALWYSNLWV (SEQ ID NO: 14).

186. The method of claim 185, wherein the bispecific antibody comprises at least one Fab molecule which specifically binds to CD3 comprising (a) a VH domain comprising an amino acid sequence having at least 95% sequence identity to the amino acid sequence of SEQ ID NO: 15; (b) a VL domain comprising an amino acid sequence having at least 95% sequence identity to the amino acid sequence of SEQ ID NO: 16; or (c) a VH domain as in (a) and a VL domain as in (b).

187. The method of claim 186, wherein the Fab molecule which specifically binds to CD3 comprises (a) a VH domain comprising the amino acid sequence of SEQ ID NO: 15 and (b) a VL domain comprising the amino acid sequence of SEQ ID NO: 16.

188. The method of claim 1, wherein the bispecific antibody:
(a) is bivalent for CD20 and monovalent for CD3; and/or
(b) a humanized antibody.

189. The method of claim 188, wherein the bispecific antibody comprises two Fab molecules which specifically bind to CD20 and one Fab molecule which specifically binds to CD3.

190. (canceled)

191. The method of claim 1, wherein the bispecific antibody is glofitamab.

192. The method of claim 1, wherein:
(a) the bispecific antibody is administered intravenously;
(b) the anti-CD20 antibody is administered intravenously; and/or
(c) ifosfamide, carboplatin, and/or etoposide are administered intravenously.

193-194. (canceled)

195. The method of claim 1, wherein the CD20-positive cell proliferative disorder is a B cell proliferative disorder.

196. The method of claim 195, wherein the B cell proliferative disorder is a non-Hodgkin's lymphoma (NHL) or a central nervous system lymphoma (CNSL).

197. The method of claim 196, wherein the NHL is a diffuse-large B cell lymphoma (DLBCL), a follicular lymphoma (FL), a mantle cell lymphoma (MCL), a marginal zone lymphoma (MZL), a high-grade B cell lymphoma, a primary mediastinal (thymic) large B cell lymphoma (PMLBCL), a diffuse B cell lymphoma, a small lymphocytic lymphoma, a Burkitt lymphoma (BL), or a Burkitt leukemia (BAL).

198. (canceled)

199. The method of claim 196, wherein the NHL is:
(a) relapsed and/or refractory; and/or
(b) aggressive and/or mature.

200. (canceled)

201. The method of claim 195, wherein the B cell proliferative disorder is a relapsed and/or refractory mature B cell NHL.

202. The method of claim 199, wherein the subject has received one prior systemic therapy.

203. The method of claim 202, wherein the subject has received no more than one prior systemic therapy.

204. The method of claim 202, wherein the prior systemic therapy comprises an anti-CD20 antibody and an anthracycline.

205. (canceled)

206. The method of claim 1, wherein the subject is transplant or CAR-T cell therapy eligible.

207. The method of claim 206, wherein the subject receives autologous stem cell transplantation (ASCT), allogenic hematopoietic stem cell transplantation, or CAR-T cell therapy after completion of the dosing regimen.

208. The method of claim 207, wherein the ASCT is an autologous hematopoietic stem cell transplant.

209-464. (canceled)
